SEQUENCE LISTING (115 sequences)

SEQ ID NO: 1 - RNA, 16 nt, synthetic construct
  misc_feature 1..16: note = tracrRNA recognition sequence
  source 1..16: mol_type = other RNA
gttttagagctatgct

SEQ ID NO: 2 - RNA, 20 nt, synthetic construct
  misc_feature 1..20: note = PAX5
  source 1..20: mol_type = other RNA
tgtgaatggacggccactcc

SEQ ID NO: 3 - RNA, 20 nt, synthetic construct
  misc_feature 1..20: note = PAX5
  source 1..20: mol_type = other RNA
tgtagtccgccagaggatag

SEQ ID NO: 4 - RNA, 20 nt, synthetic construct
  misc_feature 1..20: note = IRF8
  source 1..20: mol_type = other RNA
attgacagtagcatgtatcc

SEQ ID NO: 5 - RNA, 20 nt, synthetic construct
  misc_feature 1..20: note = IRF8
  source 1..20: mol_type = other RNA
cggaaatgtccagttgggac

SEQ ID NO: 6 - RNA, 20 nt, synthetic construct
  misc_feature 1..20: note = BACH2
  source 1..20: mol_type = other RNA
gttcctgcgcatgcacaacc

SEQ ID NO: 7 - RNA, 20 nt, synthetic construct
  misc_feature 1..20: note = BACH2
  source 1..20: mol_type = other RNA
ctgtgacgtgactttgatcg

SEQ ID NO: 8 - RNA, 20 nt, synthetic construct
  misc_feature 1..20: note = CCR5
  source 1..20: mol_type = other RNA
caatgtgtcaactcttgaca

SEQ ID NO: 9 - RNA, 20 nt, synthetic construct
  misc_feature 1..20: note = CCR5
  source 1..20: mol_type = other RNA
gctgtgtttgcgtctctccc

SEQ ID NO: 10 - RNA, 20 nt, synthetic construct
  misc_feature 1..20: note = CARD11
  source 1..20: mol_type = other RNA
caatgaccttacactgacgc

SEQ ID NO: 11 - RNA, 20 nt, synthetic construct
  misc_feature 1..20: note = PRDM1
  source 1..20: mol_type = other RNA
tgatggcggtacttcggttc

SEQ ID NO: 12 - RNA, 20 nt, synthetic construct
  misc_feature 1..20: note = PRDM1
  source 1..20: mol_type = other RNA
aggatgcggatatgactctg

SEQ ID NO: 13 - RNA, 20 nt, synthetic construct
  misc_feature 1..20: note = PRDM1
  source 1..20: mol_type = other RNA
ggggagcgagtgatgtacgt

SEQ ID NO: 14 - DNA, 4875 bp, synthetic construct
  misc_feature 1..4875: note = 1079_pscAAV-MND.GFP
  source 1..4875: mol_type = other DNA
aagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctagggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatcctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctaga

SEQ ID NO: 15 - DNA, 5056 bp, synthetic construct
  misc_feature 1..5056: note = 1347_pscAAV.Blimp.0.4kb.MND.GFP
  source 1..5056: mol_type = other DNA
aagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtgccagctgttactcaggttttctcaagaaggaggagcaactttggcagttttgcttcagttctctctagccctctgtgtaatcgcccctttttctttatttcagcacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaaacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctaga

SEQ ID NO: 16 - DNA, 6006 bp, synthetic construct
  misc_feature 1..6006: note = 1348_pAAV.Blimp.0.4kb.MND.GFP.pA
  source 1..6006: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtgccagctgttactcaggttttctcaagaaggaggagcaactttggcagttttgcttcagttctctctagccctctgtgtaatcgcccctttttctttatttcagcacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaaacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcatctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 17 - DNA, 7206 bp, synthetic construct
  misc_feature 1..7206: note = 1361_pAAV.Blimp1.1.0kb.MND.GFP.pA
  source 1..7206: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtggtaaaccatgaacatcagaaagacttttattaacctatgacagggtccccaccccagtatttttccactccattaaaatggaagttttttttttttttttcttttttgagacagagttttgctcttgttgcccagtctggagtgcaatggcacaatctcggctcaccacaacctccacctcccagattcaagcgattcttctgcctcagcctcccaagtagctgggattacaggtgtgcgccaccacgcccagctaattttgtatttttagtagagatggggtttctccatgttggtcaggctggtctcgaacttccgacctcaggtgatccgcccacctcggcctcccaaagtgctgggattacaggcaagagccactgcatccagcttaggctatcttactccagcctaaacagcaattttctatcataaggtctgtactaatgaaaacagaatcacccaaggctgctgtttgttctgtctgtgctgccattgtccgcattttgctgaggaggaaacggaactgcacttttgagtgagtggcccagagccttctagaatgagagtgcgttggaagccagatatgtggcgattgtgtcgccagctgttactcaggttttctcaagaaggaggagcaactttggcagttttgcttcagttctctctagccctctgtgtaatcgcccctttttctttatttcagcacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagagaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaaacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtaggccttgagagagagcagtccaaggggctgtgagtgcatgcttgtgtttgtatttagcttgctttccatggggtatcgattgcatttgcagtagtatgagcccccggttggggatagtgggtatggattccgcctggcttttgccacttctagctctttgactttggacaagtgacttcccttctcctctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 18 - DNA, 6707 bp, synthetic construct
  misc_feature 1..6707: note = 1366_CCR5.MND.BAFF_CRISPR.HR
  source 1..6707: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgtaccggatgcagctgctgagctgcatcgcactgagcctggcactggtgaccaacagcgcagtgcagggaccagaggagaccgtgacccaggactgcctgcagctgatcgcagacagcgagacccccaccatccagaagggcagctacaccttcgtgccctggctgctgagcttcaagcggggcagcgccctggaggagaaggagaacaagattctggtgaaggagaccggctacttcttcatctacggccaggtgctgtacaccgataagacctacgccatgggccacctgatccagcggaagaaggtgcacgtgttcggcgacgagctgagcctggtgaccctgttccggtgcatccagaacatgcccgagaccctgcccaacaacagctgctacagcgcaggaatcgcaaagctggaggagggcgacgagctgcagctggcaatcccccgggagaacgcacagatcagcctggacggcgacgtgaccttcttcggcgccctgaagctcctgtgagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 19 - DNA, 7494 bp, synthetic construct
  misc_feature 1..7494: note = 1367_CCR5.MND.BAFF.2A.GFP
  source 1..7494: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgtaccggatgcagctgctgagctgcatcgcactgagcctggcactggtgaccaacagcgcagtgcagggaccagaggagaccgtgacccaggactgcctgcagctgatcgcagacagcgagacccccaccatccagaagggcagctacaccttcgtgccctggctgctgagcttcaagcggggcagcgccctggaggagaaggagaacaagattctggtgaaggagaccggctacttcttcatctacggccaggtgctgtacaccgataagacctacgccatgggccacctgatccagcggaagaaggtgcacgtgttcggcgacgagctgagcctggtgaccctgttccggtgcatccagaacatgcccgagaccctgcccaacaacagctgctacagcgcaggaatcgcaaagctggaggagggcgacgagctgcagctggcaatcccccgggagaacgcacagatcagcctggacggcgacgtgaccttcttcggcgccctgaagctcctgggatccggtgagggcagaggaagtcttctaacatgcggtgacgtggaggagaatccgggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatctagagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 20 - RNA, 20 nt, synthetic construct
  misc_feature 1..20: note = CCR5 target guide RNA
  source 1..20: mol_type = other RNA
caatgtgtcaactcttgaca

SEQ ID NO: 21 - DNA, 20 bp, synthetic construct
  misc_feature 1..20: note = PRDM1 target guide RNA 1
  source 1..20: mol_type = other DNA
aggatgcggatatgactctg

SEQ ID NO: 22 - DNA, 20 bp, synthetic construct
  misc_feature 1..20: note = PRDM1 target guide RNA 2
  source 1..20: mol_type = other DNA
ggggagcgagtgatgtacgt

SEQ ID NO: 23 - DNA, 20 bp, synthetic construct
  misc_feature 1..20: note = IRF4g target guide RNA
  source 1..20: mol_type = other DNA
caagcaggactacaaccgcg

SEQ ID NO: 24 - RNA, 20 nt, synthetic construct
  misc_feature 1..20: note = PAX5g target guide RNA
  source 1..20: mol_type = other RNA
tgtgaatggacggccactcc

SEQ ID NO: 25 - RNA, 20 nt, synthetic construct
  misc_feature 1..20: note = BACH2 target guide RNA
  source 1..20: mol_type = other RNA
gttcctgcgcatgcacaacc

SEQ ID NO: 26 - RNA, 16 nt, synthetic construct
  misc_feature 1..16: note = tracrRNA binding sequence
  source 1..16: mol_type = other RNA
gttttagagctatgct

SEQ ID NO: 27 - DNA, 20 bp, synthetic construct
  misc_feature 1..20: note = CCR5g forward primer T7E1
  source 1..20: mol_type = other DNA
atgtgaagcaaatcgcagcc

SEQ ID NO: 28 - DNA, 30 bp, synthetic construct
  misc_feature 1..30: note = PRDM1g-1 forward primer T7E1
  source 1..30: mol_type = other DNA
tagcatttaaaaaccttgcttcttttcaag

SEQ ID NO: 29 - DNA, 21 bp, synthetic construct
  misc_feature 1..21: note = PRDM1g-2 forward primer T7E1
  source 1..21: mol_type = other DNA
cagtctaaagcaaccgagcac

SEQ ID NO: 30 - DNA, 20 bp, synthetic construct
  misc_feature 1..20: note = CCR5 reverse primer T7E1
  source 1..20: mol_type = other DNA
tcccgagtagcagatgacca

SEQ ID NO: 31 - DNA, 26 bp, synthetic construct
  misc_feature 1..26: note = PRDM1g-1 reverse primer T7E1
  source 1..26: mol_type = other DNA
tacagattctcagaggttttcagaga

SEQ ID NO: 32 - DNA, 21 bp, synthetic construct
  misc_feature 1..21: note = PRDM1g-2 reverse primer T7E1
  source 1..21: mol_type = other DNA
cttggggtagtgagcgttgta

SEQ ID NO: 33 - DNA, 20 bp, synthetic construct
  misc_feature 1..20: note = CCR5g forward primer Illumina
  source 1..20: mol_type = other DNA
ggcaacatgctggtcatcct

SEQ ID NO: 34 - DNA, 20 bp, synthetic construct
  misc_feature 1..20: note = PRDM1g-1 forward primer Illumina
  source 1..20: mol_type = other DNA
ctctcagaaggagccacagg

SEQ ID NO: 35 - DNA, 20 bp, synthetic construct
  misc_feature 1..20: note = PRDM1g-2 forward primer Illumina
  source 1..20: mol_type = other DNA
cccctcacatcagaaaagga

SEQ ID NO: 36 - DNA, 19 bp, synthetic construct
  misc_feature 1..19: note = IRF4g forward primer Illumina
  source 1..19: mol_type = other DNA
agatcgacagcggcaagta

SEQ ID NO: 37 - DNA, 20 bp, synthetic construct
  misc_feature 1..20: note = PAX5g forward primer Illumina
  source 1..20: mol_type = other DNA
cacagcggtgcttctcctat

SEQ ID NO: 38 - DNA, 20 bp, synthetic construct
  misc_feature 1..20: note = BACH2g forward primer Illumina
  source 1..20: mol_type = other DNA
tgagggattcgggacaatag

SEQ ID NO: 39 - DNA, 20 bp, synthetic construct
  misc_feature 1..20: note = CCR5g reverse primer Illumina
  source 1..20: mol_type = other DNA
ggtgaccgtcctggctttta

SEQ ID NO: 40 - DNA, 21 bp, synthetic construct
  misc_feature 1..21: note = PRDM1g-1 reverse primer Illumina
  source 1..21: mol_type = other DNA
ttggtggcatacttgaaaagc

SEQ ID NO: 41 - DNA, 20 bp, synthetic construct
  misc_feature 1..20: note = PRDM1g-2 reverse primer Illumina
  source 1..20: mol_type = other DNA
ctggagctcttgaggctttg

SEQ ID NO: 42 - DNA, 19 bp, synthetic construct
  misc_feature 1..19: note = IRF4g reverse primer Illumina
  source 1..19: mol_type = other DNA
gaggcctcctttcctcctc

SEQ ID NO: 43 - DNA, 20 bp, synthetic construct
  misc_feature 1..20: note = PAX5g reverse primer Illumina
  source 1..20: mol_type = other DNA
gcctcgagctactgccttta

SEQ ID NO: 44 - DNA, 20 bp, synthetic construct
  misc_feature 1..20: note = BACH2g reverse primer Illumina
  source 1..20: mol_type = other DNA
agttctcgcagtcctcgtgt

SEQ ID NO: 45 - DNA, 23 bp, synthetic construct
  misc_feature 1..23: note = CCR5 forward primer In-out
  source 1..23: mol_type = other DNA
ggtatgatgcttagaacagtgat

SEQ ID NO: 46 - DNA, 20 bp, synthetic construct
  misc_feature 1..20: note = ActB forward primer In-out
  source 1..20: mol_type = other DNA
actctgcaggttctatttgc

SEQ ID NO: 47 - DNA, 21 bp, synthetic construct
  misc_feature 1..21: note = CCR5 reverse primer In-out
  source 1..21: mol_type = other DNA
ccatattctgctgttccaact

SEQ ID NO: 48 - DNA, 20 bp, synthetic construct
  misc_feature 1..20: note = ActB reverse primer In-out
  source 1..20: mol_type = other DNA
aatgatctgaggagggaagg

SEQ ID NO: 49 - DNA, 21 bp, synthetic construct
  misc_feature 1..21: note = CCR5 probe In-out
  source 1..21: mol_type = other DNA
cctgggcaacatagtgtgatc

SEQ ID NO: 50 - DNA, 20 bp, synthetic construct
  misc_feature 1..20: note = ActB probe In-out
  source 1..20: mol_type = other DNA
atcaaggtgggtctctttcc

SEQ ID NO: 51 - DNA, 4875 bp, synthetic construct; identical to SEQ ID NO: 14
  misc_feature 1..4875: note = 1079_pscAAV-MND.GFP
  source 1..4875: mol_type = other DNA

SEQ ID NO: 52 - DNA, 5056 bp, synthetic construct; identical to SEQ ID NO: 15
  misc_feature 1..5056: note = 1347_pscAAV.Blimp.0.4kb.MND.GFP
  source 1..5056: mol_type = other DNA

SEQ ID NO: 53 - DNA, 6006 bp, synthetic construct; identical to SEQ ID NO: 16
  misc_feature 1..6006: note = 1348_pAAV.Blimp.0.4kb.MND.GFP.pA
  source 1..6006: mol_type = other DNA

SEQ ID NO: 54 - DNA, 7206 bp, synthetic construct; identical to SEQ ID NO: 17
  misc_feature 1..7206: note = 1361_pAAV.Blimp1.1.0kb.MND.GFP.pA
  source 1..7206: mol_type = other DNA

SEQ ID NO: 55 - DNA, 6707 bp, synthetic construct; identical to SEQ ID NO: 18
  misc_feature 1..6707: note = 1366_CCR5.MND.BAFF_CRISPR.HR
  source 1..6707: mol_type = other DNA

SEQ ID NO: 56 - DNA, 7494 bp, synthetic construct; identical to SEQ ID NO: 19
  misc_feature 1..7494: note = 1367_CCR5.MND.BAFF.2A.GFP
  source 1..7494: mol_type = other DNA

SEQ ID NO: 57 - DNA, 8330 bp, synthetic construct
  misc_feature 1..8330: note = 1378_CCR5.MND.mCherry.2A.coFIXpadua.WPRE.pa
  source 1..8330: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggatccggtgagggcagaggaagtcttctaacatgcggtgacgtggaggagaatccgggccccatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctgagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 58 - DNA, 7806 bp, synthetic construct
  misc_feature 1..7806: note = 1376_CCR5.MND.FiX.coFIXpadua.WPRE.pA
  source 1..7806: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgagatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctgagtcgacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 59 - DNA, 6968 bp, synthetic construct
  misc_feature 1..6968: note = pAAV_CCR5.MND.II2ss.ADP.mAPRIL
  source 1..6968: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgagatgtatcggatgcagctcttgagctgtatcgctctgtcactggcacttgttaccaactcagaggatgacgttaccaccacggaagaacttgcgcccgctttggtaccgcctccgaaaggaacctgtgccggttggatggctggaataccaggacatcccggacacaatggaacgccaggacgggatggacgcgacggcacgcccggagaaaaaggggagaaaggggatgcaggcttgctcgggccaaagggcgaaaccggcgacgttggaatgacaggcgctgaaggacctcggggttttccgggaaccccgggccgcaagggcgaacctggcgaggccgccgcagtattgacacagaaacagaaaaagcaacattccgtccttcatctggtccccatcaacgcaacctccaaggatgatagtgatgtgaccgaggtaatgtggcaacccgcgcttaggcgaggaagaggtctgcaggcgcagggatacggggtgcgaatccaagatgctggggtgtacctgctgtactcacaggttttgtttcaggacgtaacatttacgatggggcaggtcgtgtcccgagaaggacaagggagacaggaaacactcttccggtgtattagaagtatgccttcacatcctgatcgcgcttacaactcttgttattccgctggcgtctttcacttgcatcagggcgacatcctttcagtgataattccgagagcgcgggctaagttgaatcttagcccccacggcacatttctcggattcgtgaagctttgatgagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 60 - DNA, 7620 bp, synthetic construct
  misc_feature 1..7620: note = pAAV CCR5.MND.IL6.Frun.T2A.GFP
  source 1..7620: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgagatgaacagtttttctacttctgccttcggacccgtcgcctttagcctgggcctgctgctggtgctgcctgccgcattccccgctcctgtgccccctggagaagactccaaggatgtcgccgctcctcacagacagccactgactagctccgagaggatcgacaaacagatccgctacattctggatggcattagcgccctgcgcaaggaaacctgcaacaaatccaatatgtgtgagtctagtaaggaagcactggccgagaacaatctgaacctgcccaagatggctgagaaagacggctgcttccagtctgggtttaatgaggaaacctgtctggtgaaaatcattacagggctgctggagttcgaagtctacctggaatatctgcagaaccgatttgagtcaagcgaggaacaggctcgggcagtgcagatgagcacaaaggtcctgatccagttcctgcagaagaaagccaaaaatctggacgctattaccacaccagatcccactaccaacgcttctctgctgaccaagctgcaggcacagaatcagtggctgcaggatatgacaactcacctgatcctgaggagtttcaaagaatttctgcagtcctctctgcgggcactgagacagatgcgcgctaagcgaggatccggtgagggcagaggaagtcttctaacatgcggtgacgtggaggagaatccgggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatctagagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 61 - DNA, 7764 bp, synthetic construct
  misc_feature 1..7764: note = pAAV CCR5.MND.II2ss-ADP.mAPRIL.Furin.T2A.GFP
  source 1..7764: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgagatgtatcggatgcagctcttgagctgtatcgctctgtcactggcacttgttaccaactcagaggatgacgttaccaccacggaagaacttgcgcccgctttggtaccgcctccgaaaggaacctgtgccggttggatggctggaataccaggacatcccggacacaatggaacgccaggacgggatggacgcgacggcacgcccggagaaaaaggggagaaaggggatgcaggcttgctcgggccaaagggcgaaaccggcgacgttggaatgacaggcgctgaaggacctcggggttttccgggaaccccgggccgcaagggcgaacctggcgaggccgccgcagtattgacacagaaacagaaaaagcaacattccgtccttcatctggtccccatcaacgcaacctccaaggatgatagtgatgtgaccgaggtaatgtggcaacccgcgcttaggcgaggaagaggtctgcaggcgcagggatacggggtgcgaatccaagatgctggggtgtacctgctgtactcacaggttttgtttcaggacgtaacatttacgatggggcaggtcgtgtcccgagaaggacaagggagacaggaaacactcttccggtgtattagaagtatgccttcacatcctgatcgcgcttacaactcttgttattccgctggcgtctttcacttgcatcagggcgacatcctttcagtgataattccgagagcgcgggctaagttgaatcttagcccccacggcacatttctcggattcgtgaagcttcgcgctaagcgaggatccggtgagggcagaggaagtcttctaacatgcggtgacgtggaggagaatccgggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatctagagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 62 - DNA, 6821 bp, synthetic construct
  misc_feature 1..6821: note = pAAV CCR5 MND IL6
  source 1..6821: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgagatgaacagtttttctacttctgccttcggacccgtcgcctttagcctgggcctgctgctggtgctgcctgccgcattccccgctcctgtgccccctggagaagactccaaggatgtcgccgctcctcacagacagccactgactagctccgagaggatcgacaaacagatccgctacattctggatggcattagcgccctgcgcaaggaaacctgcaacaaatccaatatgtgtgagtctagtaaggaagcactggccgagaacaatctgaacctgcccaagatggctgagaaagacggctgcttccagtctgggtttaatgaggaaacctgtctggtgaaaatcattacagggctgctggagttcgaagtctacctggaatatctgcagaaccgatttgagtcaagcgaggaacaggctcgggcagtgcagatgagcacaaaggtcctgatccagttcctgcagaagaaagccaaaaatctggacgctattaccacaccagatcccactaccaacgcttctctgctgaccaagctgcaggcacagaatcagtggctgcaggatatgacaactcacctgatcctgaggagtttcaaagaatttctgcagtcctctctgcgggcactgagacagatgtgagtcgactgctttatttgtgaaatttgtgatgctattgctttatttgtaaccattataagctgcaataaacaagttaacaacaacaattgcattcattttatgtttcaggttcagggggaggtgtgggaggttttttaaactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacaccgcggtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 63 - DNA, 20 bp, synthetic construct
  misc_feature 1..20: note = antisense gRNA
  source 1..20: mol_type = other DNA
ggactccccagaaaagcaaa

SEQ ID NO: 64 - DNA, 20 bp, synthetic construct
  misc_feature 1..20: note = sense gRNA
  source 1..20: mol_type = other DNA
aagaaccatttgcttttctg

SEQ ID NO: 65 - DNA, 20 bp, synthetic construct
  misc_feature 1..20: note = sense gRNA
  source 1..20: mol_type = other DNA
tttgcttttctggggagtcc

SEQ ID NO: 66 - DNA, 20 bp, synthetic construct
  misc_feature 1..20: note = sense gRNA
  source 1..20: mol_type = other DNA
attaaggctgttcatgtgaa

SEQ ID NO: 67 - DNA, 20 bp, synthetic construct
  misc_feature 1..20: note = antisense gRNA
  source 1..20: mol_type = other DNA
ccggcgcggcaggcgcatgg

SEQ ID NO: 68 - DNA, 20 bp, synthetic construct
  misc_feature 1..20: note = antisense gRNA
  source 1..20: mol_type = other DNA
agccccagcgcggcccggcg

SEQ ID NO: 69 - DNA, 20 bp, synthetic construct
  misc_feature 1..20: note = sense gRNA
  source 1..20: mol_type = other DNA
tgccgcgccgggccgcgctg

SEQ ID NO: 70 - DNA, 20 bp, synthetic construct
  misc_feature 1..20: note = antisense gRNA
  source 1..20: mol_type = other DNA
aagcggcaggagccccagcg

SEQ ID NO: 71 - DNA, 20 bp, synthetic construct
  misc_feature 1..20: note = sense gRNA
  source 1..20: mol_type = other DNA
aggtagacaattgcagcctg

SEQ ID NO: 72 - DNA, 20 bp, synthetic construct
  misc_feature 1..20: note = sense gRNA
  source 1..20: mol_type = other DNA
tccctacagacagagccaca

SEQ ID NO: 73 - DNA, 20 bp, synthetic construct
  misc_feature 1..20: note = sense gRNA
  source 1..20: mol_type = other DNA
agatgttgtcctgacacttg

SEQ ID NO: 74 - DNA, 20 bp, synthetic construct
  misc_feature 1..20: note = sense gRNA
  source 1..20: mol_type = other DNA
gccttcaccatgaagtccag

SEQ ID NO: 75 - DNA, 20 bp, synthetic construct
  misc_feature 1..20: note = sense gRNA
  source 1..20: mol_type = other DNA
ttcaactgctcatcagatgg

SEQ ID NO: 76 - DNA, 20 bp, synthetic construct
  misc_feature 1..20: note = antisense gRNA
  source 1..20: mol_type = other DNA
ggccaaagtacagtggaagg

SEQ ID NO: 77 - DNA, 20 bp, synthetic construct
  misc_feature 1..20: note = sense gRNA
  source 1..20: mol_type = other DNA
cacgctgctcgtatccgacg

SEQ ID NO: 78 - DNA, 20 bp, synthetic construct
  misc_feature 1..20: note = antisense gRNA
  source 1..20: mol_type = other DNA
ccttccaaggacgtcatgca

SEQ ID NO: 79 - DNA, 20 bp, synthetic construct
  misc_feature 1..20: note = antisense gRNA
  source 1..20: mol_type = other DNA
cagccttaataaaaaccgcc

SEQ ID NO: 80 - DNA, 20 bp, synthetic construct
  misc_feature 1..20: note = sense gRNA
  source 1..20: mol_type = other DNA
ttcttccgaagatcctaatg

SEQ ID NO: 81 - DNA, 20 bp, synthetic construct
  misc_feature 1..20: note = antisense gRNA
  source 1..20: mol_type = other DNA
gtcaggatagcaggcatctg

SEQ ID NO: 82 - DNA, 20 bp, synthetic construct
  misc_feature 1..20: note = antisense gRNA
  source 1..20: mol_type = other DNA
tactggctccacttctcgag

SEQ ID NO: 83 - DNA, 20 bp, synthetic construct
  misc_feature 1..20: note = sense gRNA
  source 1..20: mol_type = other DNA
gatattgatatacttcctag

SEQ ID NO: 84 - DNA, 20 bp, synthetic construct
  misc_feature 1..20: note = antisense gRNA
  source 1..20: mol_type = other DNA
acccaaaggggtctcaaagg

SEQ ID NO: 85 - DNA, 20 bp, synthetic construct
  misc_feature 1..20: note = antisense gRNA
  source 1..20: mol_type = other DNA
cagcgtcttagcacccaaag

SEQ ID NO: 86 - DNA, 20 bp, synthetic construct
  misc_feature 1..20: note = sense gRNA
  source 1..20: mol_type = other DNA
cgaatgcaatcagatgctag

SEQ ID NO: 87 - DNA, 20 bp, synthetic construct
  misc_feature 1..20: note = antisense gRNA
  source 1..20: mol_type = other DNA
accgagacagtcgggaccgt

SEQ ID NO: 88 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
ggactccccagaaaagcaaagttttagagctatgct

SEQ ID NO: 89 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
aagaaccatttgcttttctggttttagagctatgct

SEQ ID NO: 90 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
tttgcttttctggggagtccgttttagagctatgct

SEQ ID NO: 91 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  misc_feature 34..36: note = m-2'O-methyl-modified-base-phosphorothioate-bond
  source 1..36: mol_type = other RNA
attaaggctgttcatgtgaagttttagagctatgct

SEQ ID NO: 92 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
ccggcgcggcaggcgcatgggttttagagctatgct

SEQ ID NO: 93 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
agccccagcgcggcccggcggttttagagctatgct

SEQ ID NO: 94 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
tgccgcgccgggccgcgctggttttagagctatgct

SEQ ID NO: 95 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
aagcggcaggagccccagcggttttagagctatgct

SEQ ID NO: 96 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
aggtagacaattgcagcctggttttagagctatgct

SEQ ID NO: 97 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
tccctacagacagagccacagttttagagctatgct

SEQ ID NO: 98 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
agatgttgtcctgacacttggttttagagctatgct

SEQ ID NO: 99 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
gccttcaccatgaagtccaggttttagagctatgct

SEQ ID NO: 100 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
ttcaactgctcatcagatgggttttagagctatgct

SEQ ID NO: 101 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
ggccaaagtacagtggaagggttttagagctatgct

SEQ ID NO: 102 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
cacgctgctcgtatccgacggttttagagctatgct

SEQ ID NO: 103 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
ccttccaaggacgtcatgcagttttagagctatgct

SEQ ID NO: 104 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
cagccttaataaaaaccgccgttttagagctatgct

SEQ ID NO: 105 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
ttcttccgaagatcctaatggttttagagctatgct

SEQ ID NO: 106 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
gtcaggatagcaggcatctggttttagagctatgct

SEQ ID NO: 107 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
tactggctccacttctcgaggttttagagctatgct

SEQ ID NO: 108 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
gatattgatatacttcctaggttttagagctatgct

SEQ ID NO: 109 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
acccaaaggggtctcaaagggttttagagctatgct

SEQ ID NO: 110 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
cagcgtcttagcacccaaaggttttagagctatgct

SEQ ID NO: 111 - RNA, 36 nt, synthetic construct
  misc_feature 1..36: note = sense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
cgaatgcaatcagatgctaggttttagagctatgct

SEQ ID NO: 112 - RNA, 36 nt, synthetic construct
  misc_feature 1..36: note = antisense gRNA - mod and linker
  modified_base 1..3: mod_base = OTHER; note = note = m-2'O-methyl-modified-base-phosphorothioate-bond
  modified_base 34..36: mod_base = OTHER; note = note = m-2'O-methyl-modified-base-phosphorothioate-bond
  source 1..36: mol_type = other RNA
accgagacagtcgggaccgtgttttagagctatgct

SEQ ID NO: 113 - DNA, 79 bp, Homo sapiens
  misc_feature 1..79: note = Portion of wild type PRDM1 gene
  source 1..79: mol_type = unassigned DNA
agaagactttttgaaagcttccctggcctacgggatcgagagacccacgtacatcactcgctcccccattccatcctcc

SEQ ID NO: 114 - DNA, 76 bp, Homo sapiens
  misc_feature 1..76: note = Portion of PRDM1 gene with deletion
  source 1..76: mol_type = unassigned DNA
agaagactttttgaaagcttccctggcctacgggatcgagagacccacgatcactcgctcccccattccatcctcc

SEQ ID NO: 115 - DNA, 79 bp, Homo sapiens
  misc_feature 1..79: note = Portion of PRDM1 gene with homologous directed repair
  source 1..79: mol_type = unassigned DNA
agaagactttttgaaagcttccctggcctacgggatcgagagaaccacgtacatcactcgctcccccattccatcctcc

(19) United States
(12) Patent Application Publication
(10) Pub. No.: US 2024/0287452 A1
(43) Pub. Date: Aug. 29, 2024

(54) ENGRAFTABLE CELL-BASED IMMUNOTHERAPY FOR LONG-TERM DELIVERY OF THERAPEUTIC PROTEINS

(71) Applicant: Seattle Children's Hospital (dba Seattle Children's Research Institute), Seattle, WA (US)

(72) Inventors: David J. Rawlings, Seattle, WA (US); Richard James, Seattle, WA (US); Shaun W. Jackson, Seattle, WA (US); Iram Khan, Issaquah, WA (US); King Hung, Seattle, WA (US); Andrew M. Scharenberg, Seattle, WA (US)

(21) Appl. No.: 18/441,393

(22) Filed: Feb. 14, 2024

Related U.S. Application Data
(63) Continuation of application No. 15/921,353, filed on Mar. 14, 2018, now Pat. No. 11,939,594.
(60) Provisional application No. 62/472,493, filed on Mar. 16, 2017.
(60) Provisional application No. 62/549,385, filed on Aug. 23, 2017.
(60) Provisional application No. 62/571,918, filed on Oct. 13, 2017.

(51) Int. Cl. C12N 5/078; A61K 35/17; A61K 48/00; C07K 14/00; C07K 16/00; C12N 5/0781; C12N 9/22; C12N 15/11; C12N 15/113; C12N 15/90

(52) U.S. Cl. CPC C12N 5/0634; A61K 48/0058; C07K 14/00; C07K 16/00; C12N 5/0635; C12N 9/22; C12N 15/11; C12N 15/90; A61K 35/17; C07K 2317/14; C07K 2317/21; C12N 15/113; C12N 2310/20; C12N 2501/056; C12N 2501/2302; C12N 2501/2306; C12N 2501/231; C12N 2501/2315; C12N 2501/24; C12N 2501/52; C12N 2506/11; C12N 2510/00; C12N 2510/02; C12N 2750/14143; C12N 2800/80

(57) ABSTRACT

The present application relates to plasma cells and plasma cell precursors that express a macromolecule, such as a protein, protein mimetic or a peptide and compositions comprising these plasma cells or plasma cell precursors. The application further relates to methods of using and making the plasma cells and plasma cell precursors that express the macromolecule. Methods of treatment comprising administering the plasma cells or plasma cell precursors are also contemplated.

### CROSS REFERENCE TO RELATED APPLICATIONS

The present application is a continuation of U.S. application Ser. No. 15/921,353 filed Mar. 14, 2018 which claims the benefit of priority to U.S. Prov. App. No. 62/472,493, filed Mar. 16, 2017, U.S. Prov. App. No. 62/549,385, filed Aug. 23, 2017, and U.S. Prov. App. No. 62/571,918, filed Oct. 13, 2017 which are each expressly incorporated by reference in its entirety.

### REFERENCE TO SEQUENCE LISTING

This application incorporates by reference the Sequence Listing provided as an XML file entitled SCRI133C1SEQLIST.XML created Apr. 22, 2024, and is 296,563 bytes in size. The information in the electronic format of the Sequence Listing is incorporated herein by reference in its entirety.

### FIELD OF THE INVENTION

A new approach for engineering human plasma cells or plasma cell precursors to express macromolecules such as proteins for therapeutic purposes, has been discovered. The alternatives described herein include methods requiring isolation and activation of B cells from subject-derived (autologous) or allogeneic peripheral blood mononuclear cells; genome engineering of these B cells or B cell precursors so that they express molecules, such as macromolecules, protein mimetics, proteins or peptides of interest in the absence of viral integration (with or without additional genetic modifications that modulate their eventual function and survival); expansion and differentiation of these cells into >1e⁻⁶ long-lived plasma cells in vitro; and administration of these autologous or allogeneic engineered protein-producing plasma cells into human recipients for therapeutic application.

### BACKGROUND OF THE INVENTION

To date there are limited reports of genome editing in primary B cells. These limited reports can be found, for example, in Cheong et al. 2016 and Chu et al. 2016 (“Editing of mouse and human immunoglobulin genes by CRISPR-Cas9 system.” Nat Commun. 2016 Mar. 9; 7:10934. and “Efficient CRISPR-mediated mutagenesis in primary immune cells using CrispRGold and a C57BL/6 Cas9 transgenic mouse line.” Proc Natl Acad Sci USA. 2016 Nov. 1; 113(44):12514-12519.; both incorporated by reference in their entireties herein). These reports have used either transient transfection of plasmid DNA or lentiviral vector delivery to facilitate gene disruption. However, there are no reports available in the medical literature of using nucleases to achieve homologous recombination in primary human B cells.

One of the main problems with the use of plasmids is their low efficiency. For example, plasmid-based methods of DNA delivery to primary B cells are extremely toxic, which is likely due to innate DNA sensing and they also exhibit low efficiency (<1%). Lentiviral-based gene delivery also has low efficiency in these cells, for example, in most cases less than 5% of the cells can be transduced, and gene targeting in primary B cells has historically required use of cells from animals with transgenic expression of CAS9. In contrast, the genome engineering approach used in the alternatives described herein enables one to selectively edit the genes with introduction of gene expression cassettes by homologous recombination in more than 30% of primary human B cells and to selectively expand these cells to generate an enriched cell product expressing transgenes of interest.

Selectivity is also an issue with lentiviral and plasmid DNA. For example, lentiviral vectors integrate randomly and primarily in sites of active transcription and have the potential to be oncogenic. In contrast, nuclease targeting and homology directed repair (HDR) dependent integration of payload allow for selectivity with minimal off-target effects. The need for new approaches that allow one to achieve homologous recombination in primary human B cells is manifest.

### SUMMARY OF THE INVENTION

Aspects of the alternatives described herein include, but are not limited to: (1) the use of blood-derived human B cells and/or B cell precursors as a starting material for a plasma cell molecule producing immunotherapy; (2) RNA- and protein-based transfection to facilitate delivery of candidate designer nucleases targeting a broad range of genetic loci in primary B cells that include, but are not limited to zinc finger nucleases, transcription activator-like effector nucleases (TALEN), homing endonucleases (HEs), combined TALEN-HE proteins (megaTALs) and CRISPR/Cas systems; (3) transfection of long single-stranded DNA oligonucleotides or transduction with recombinant adeno-associated virus to facilitate efficient delivery of donor DNA templates carrying therapeutic expression cassettes into primary human B cells and/or B cell precursors in order to facilitate efficient homologous recombination into a range of candidate genetic loci; (4) integrity measures that include, but are not limited to, methods to prevent somatic hypermutation of the B cell antibody locus during the engineering process including, but not limited, to disruption of the AID gene; (5) production enhancers that include, but are not limited to methods to introduce dimerizable drug-inducible activating proteins to enable selectable expansion of engineered human B cells and/or B cell precursors in vitro or in vivo; (6) safety measures that include, but are not limited to, introduction of sequences from cell surface proteins including, but not limited to, the CD20 protein into B cells and/or B cell precursors to enable targeted removal from recipients using rituxan® or alternative therapeutic approaches; and (7) the use of a proprietary multi-step cytokine and co-culture based systems to facilitate differentiation of blood-derived B cells and/or B cell precursors into long-lived plasma cells and their survival and expansion in vitro. The human B cells as described herein, can include B cell precursors such as hematopoietic stem cells (HSCs), multipotent progenitor (MPP) cells, lymphoid progenitor (CLP) cells, naïve B cells, GC B cell, plasmablast, early pro-B cells, late pro-B cells, large pre-B cells, small pre-B cells, immature B cells, T1 B cells, T2 B cells, marginal zone B cells, mature B cells and/or memory B cells. Without being limiting, the molecule comprises macromolecules, proteins, protein mimetics and peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, of the CRISPR/Cas system described herein, the Cas nuclease comprises Cas1, Cas2, Cas3, Cas4, Cas5, Cas6, Cas7, Cas8 or Cas9.

Multiple categories of use of a plasma cell producing a molecule, such as a macromolecule, protein or a peptide in immunotherapy are also envisioned. Exemplary alternatives are listed below:
(1) Prophylactic or therapeutic protection from infection (viral, bacterial, or parasitic) in healthy subjects or following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites;
(2) Protein replacement, enzyme replacement and rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia);
(3) Immune modulation via expressed cytokines, cytokine receptors, complement proteins or other inhibitory proteins including, but not limited to: Ill receptor antagonist for treatment or amelioration of periodic fever/autoinflammatory syndromes; complement inhibitory proteins (including Factor H, Factor I) for treatment or inhibition of atypical hemolytic uremic syndrome/membranoproliferative glomerulonephritis; and/or C1 inhibitor for hereditary angioedema;
(4) Anti-fibrotic molecules including, but not limited to SCGB1A1 for the treatment of pulmonary fibrosis;
(5) Therapeutic antibodies or at least one binding portion thereof for treating or ameliorating autoimmune disorders, autoinflammatory disorders, immune dysregulation and/or cancer including but not limited to: anti-IL1 monoclonal antibodies or a binding portion thereof for the treatment or amelioration of periodic fever/autoinflammatory syndromes; anti-TNF antibodies or a binding portion thereof for the treatment or inhibition of inflammatory arthritis/inflammatory bowel disease, anti-IL33 antibodies or a binding portion thereof for the treatment or inhibition of asthma and/or anti-C5 antibodies or a binding portion thereof for the treatment or inhibition of paroxysmal nocturnal hemoglobinuria/atypical HUS;
(6) Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein);
(7) Glucose response elements upstream of insulin for the treatment or inhibition of diabetic conditions; and
(8) Therapeutic monoclonal antibodies for hyper-cholesterolemia, including anti-PCSK9 inhibitory antibodies or a binding portion thereof. Preferred alternatives include the following alternatives. In some alternatives herein, the protein is a neutralizing antibody that block influenza, parainfluenza, rhinovirus, Respiratory Syncitial Virus (RSV), HIV, pathogenic bacteria, and/or parasites. In some alternatives herein, the protein is an enzyme. In some alternatives, the protein is Factor VIII, Factor IX, SERPING1 or SERPINA1. In some alternatives, the protein is IFN-alpha, Factor VIII, Factor IX, SERPING1 or SERPINAL. In some alternatives, the enzyme is ADAMTS13, LIPA, GLA, or ALPL. In some alternatives, the plasma cell expresses cytokines, cytokine receptors, complement proteins or other inhibitory proteins.

In a first aspect, a method of making plasma cells or plasma cell precursors that express a molecule is provided, the method comprising: (a) isolating B cells, (b) developing the B cells, (c) performing a first round of genome editing of the B cells for protein expression in absence of viral integration, (d) expanding the B cells and (e) differentiating the B cells, optionally, after step (c) or (d), thereby producing plasma cells or plasma cell precursors that express the molecule. In some alternatives, the molecule is a protein, protein mimetic or peptide. In some alternatives, the molecule is Factor VIII, Factor IX, SERPING1, SERPINA1, complement inhibitory protein, Factor H, Factor I, a C1 inhibitor, an anti-fibrotic molecule, SCGBlA1, a therapeutic antibody or a binding portion thereof, an anti-IL-1 monoclonal antibody, an anti-TNF antibody, an anti-IL-33 antibody, an anti-C5 antibody, an anti-thrombotic molecule, APLN, an anti-PCSK9 inhibitory antibody or binding portion thereof, a neutralizing HIV-1 antibody (bNAbs) or binding portion thereof, IFN-alpha, BAFF, APRIL, IL-10, IL-6, ADAMTS13, LIPA, GLA or ALPL. In some alternatives, the developing of the B cells is performed after the B cells are arrested at a specific phase of development or activating the B cells such that the B cells are permissive for recombination without further B cell differentiation, wherein the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration, and wherein performing the first round of genome editing of the B cells for protein expression is performed by introduction of a single stranded nucleic acid. In some alternatives, the first round of genome editing is performed by an RNA and protein based transfection and, wherein the first round of genome editing comprises delivering a nuclease, wherein the nuclease targets at least one genetic loci in the B cell and, wherein the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the first round of genome editing comprises transducing the B cell with a recombinant adeno-associated virus vector to serve as a donor template for homologous recombination into a candidate genetic loci. In some alternatives, the method further comprises preventing somatic hypermutation of an antibody locus in the B cell. In some alternatives, the method further comprises selectively increasing a proportion of gene edited B cells and, wherein the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, step (a) further comprises removing IgM positive cells. In some alternatives, the method further comprises introducing a nucleic acid, wherein the nucleic acid encodes at least one cell surface protein. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 or IL-15, wherein the plasmablast differentiation step is performed in the presence any combination of IL-2, IL-6, IL-10 or IL-15 or wherein the plasma cell differentiation step is performed in the presence any combination of IL-6, IL-15, APRIL or IFNα. In some alternatives, the plasma cells are long lived plasma cells. In some alternatives, the method further comprises purifying the plasma cells by positive selection against CD138.

In second aspect, a composition comprising a plasma cell is provided, which expresses a molecule, wherein said molecule is a heterologous protein, protein mimetic or a peptide. In some alternatives, the molecule comprises an enzyme, neutralizing antibody or binding portion thereof, cytokine, cytokine receptor, complement protein, inhibitory protein, anti-fibrotic molecule, therapeutic antibody or binding portion thereof, anti-thrombotic molecule, glucose response element, or a monoclonal antibody or binding portion thereof. In some alternatives, the composition further comprises a second B cell, wherein the second B cell secretes a molecule that induces tolerance of a peptide or that induces engraftment of the plasma cell.

In a third aspect, a method of expressing a molecule in a subject is provided comprising: administering the composition of anyone of the alternatives herein to the subject. In some alternatives, the composition comprisesa plasma cell is provided, which expresses a molecule, wherein said molecule is a heterologous protein, protein mimetic or a peptide. In some alternatives, the molecule comprises an enzyme, neutralizing antibody or binding portion thereof, cytokine, cytokine receptor, complement protein, inhibitory protein, anti-fibrotic molecule, therapeutic antibody or binding portion thereof, anti-thrombotic molecule, glucose response element, or a monoclonal antibody or binding portion thereof. In some alternatives, the composition further comprises a second B cell, wherein the second B cell secretes a molecule that induces tolerance of a peptide or that induces engraftment of the plasma cell. In some alternatives, the subject has received a stem cell administration or a solid organ transplantation or is a subject identified or selected as one to receive a stem cell administration or a solid organ transplantation or, wherein the subject has an enzyme deficiency, pulmonary fibrosis, an autoimmune disorder, immune dysregulation, cancer, diabetes, HIV or hypercholesterolemia.

### BRIEF DESCRIPTION OF THE DRAWINGS

FIG. 1 shows a schematic of the potential clinical application of plasma cell therapy.

FIG. 2 highlights the advantages of using engineered plasma cells for a protein delivery platform, such as longevity, protein production and its potential for ex vivo manipulation and generation.

FIG. 3 shows candidate nuclease platforms that can be used in the alternatives described herein, such as, for example, zinc finger nucleases, CRISPR/Cas Nuclease systems, TAL effector nucleases and homing endonucleases.

FIG. 4 provides a table that shows different methods of CRISPR editing of the primary B cells and the benefits of the methods.

FIG. 5 shows the ribonucleoprotein strategy for the editing of B cells.

FIG. 6 shows the indel in a Sanger sequencing gel. As shown, increasing the concentration of ribonuclease increased the T7 cleavage during a 5 day DNA cleavage protocol. For the experiment, B cells were isolated from peripheral blood mononuclear cells at day zero and transfected with ribonucleoproteins and a PR domain zinc finger protein at day 1. The cells were then activated with mCD40L, CpG and IL2, IL10, and IL15. At day 5, the DNA was extracted from the cell to test for DNA cleavage.

FIG. 7 shows a Western blot, which illustrates results from a T7 cleavage. The antibodies were directed to PRDM1 and TUBB1.

FIG. 8 shows that PRDM1 disruption limits plasmablast generation.

FIG. 9 shows homologous-directed repair (HDR) using single-stranded DNA templates.

FIG. 10 shows homologous-directed repair (HDR) using single-stranded DNA templates.

FIG. 11 shows dose response of short oligonucleotide HDR.

FIG. 12 shows low toxicity in setting of high-efficiency HDR.

FIG. 13 shows long oligonucleotide templates for HDR.

FIG. 14 shows that long template HDR is effective.

FIG. 15 shows results from the testing of primary B cell transduction with AAV vectors. Provided in the right panel is the percent GFP expression with different AAV serotypes. From left to right are AAV serotypes by culture volume from 2%, 5% and 10%. The bottom panel shows the percent viability of the cells after 48 hours post transduction. From left to right consecutively in the graph are the AAV amount by culture volume of 2%, 5% and 10%.

FIG. 16 shows results from the testing of primary B cell transduction with AAV vectors.

FIG. 17 shows results from transduction of B cells using AAV6.

FIG. 18 shows the strategy of homologous-directed repair (HDR) using AAV repair templates.

FIG. 19 shows that CRISPR and AAV repair template co-delivery mediates HDR in primary B cells.

FIG. 20 shows the strategy of Homologous-directed repair (HDR) using AAV repair templates.

FIG. 21 shows CRISPR and AAV repair template co-delivery mediates HDR in primary B cells.

FIG. 22 shows the culturing and genome editing strategy using negative selection isolation of B cells from healthy donor PBMCs.

FIG. 23 shows B cell phenotype at end of expansion phase.

FIG. 24 shows B cell expansion results. The left panel shows the cell expansion curve and the right panel shows the viability by DAPI staining.

FIG. 25 shows Plasma cell differentiation using the 3-step culture system.

FIG. 26 shows results of plasma cell differentiation at pre-differentiation and at post-differentiation (day 12).

FIG. 27 shows the demonstration of quantifiable secretion of an exogenous protein in an animal model.

FIG. 28 shows the demonstration of the ability of B cells to produce functionally active Factor IX.

FIG. 29A and FIG. 29B shows sustained expression of human proteins in murine serum between 10 and 21 days.

FIG. 30 shows the experimental design for gene disruption and protein expression.

FIG. 31 shows the long lived human antibody production in mice.

FIG. 32 shows expression of human IL6 in mice confers a growth advantage for transplanted ex vivo differentiated human plasma cells.

FIG. 33 shows high IL6 expression correlates with high plasma cell secretion.

FIG. 34 shows in vitro human APRIL treatment correlates with increased plasma cell antibody secretion in vivo.

FIG. 35 shows the results of the gene disruption and protein expression.

FIG. 36 shows targeting of abundant plasma cell genes using CRISPR.

FIG. 37 shows multiplexed genome editing to introduce FIX and increase protein secretion in human plasma cells.

FIG. 38 shows knock-out of BANK1 promotes plasma cell differentiation and antibody production.

FIG. 39 shows long-lived plasma cells secrete more immunoglobulin on a per cell basis.

FIGS. 40A-40C depict B cell activation cocktail leads to expansion of naïve B cells in vitro. Primary human CD19+ B cells were isolated from PBMCs and cultured with MEGACD40L®, CpG, IL2, IL10 and IL15 for 13 days. Fresh medium with the soluble factors was replenished every one or two days to maintain a cell density of ˜-1×10⁶ cells/ml. FIG. 40A and FIG. 40B depict line graphs for normalized cell number in which cells were counted every day, and for percentage viability measured using 4′,6-diamidino-2-phenylindole (DAPI) staining (n=2), respectively. Line graphs show means±SEM. FIG. 40C depicts a flow cytometry analysis in which cell samples were stained using anti-CD27, CD138, CD19, CD38, IgD and IgM antibodies after 0, 2, 8, and 13 days of culture. Immunophenotypes were analyzed by flow cytometry.

FIG. 41 shows primary B cells are effectively electroporated with mRNA encoding blue fluorescent protein (BFP). B cells were activated for two days in culture and transfected with BFP mRNA using the optimal electroporation settings. Percentages of BFP+ cells were measured 24 hours after transfection by flow cytometry. Left: representative flow plots showing BFP expression of mock and BFP mRNA electroporated B cells. Right: bar graph showing percentages of BFP+ cells (n=5). All values are means±SEM.

FIGS. 42A-42D depict Cas9 RNP induces site-specific indels or a precise single-nucleotide change in the presence of an ssODN in primary human B cells. FIG. 42A depicts results of an experiment in which CD19+ B cells were isolated and activated in vitro for two days, mock treated or transfected with Cas9 RNPs targeting CCR5 or PRDM1 (CCR5g, PRDM1g-1 or PRDM1g-2) and cultured for five additional days. Total genomic DNA was isolated on day 5, target regions were PCR amplified and analyzed using the T7 endonuclease 1 (T7E1) assay (one representative experiment shown) or sequencing for percentages of on-target indels (112,000 reads per experimental condition). FIG. 42B depicts a diagram of wild type PRDM1 locus, PRDM1g-2 target location and the ssODN donor template containing a single-nucleotide change. (42C, D) B cells were activated for two days and mock treated, electroporated with Cas9 RNP-PRDM1g-2 alone, or with the ssODN donor template at serially increasing doses from 7.5 pmol to 120 pmol. FIG. 42C depicts a graph for viabilities of B cells 2 days and 5 days after genome editing (n=3, three donors). No significant difference in viability was observed between mock and up to 30 pmol ssODN plus RNP either on day 2 or day 5. The bar graph shows means±SEM. FIG. 42D depicts a graph of percentage of HDR, indel and wildtype (WT) alleles in total genomic DNA extracted 5 days after genome editing as assessed by sequencing (greater than 1 million reads per experimental condition). N represents the number of independent experiments. One-way ANOVA with the Sidak correction for multiple comparisons is used; ***p<0.001; ns, not significant. WT, wildtype. As shown in FIG. 42D of the bar graphs, the top black portions of the bar are wild type, light grey is HDR and the bottom grey is indel.

FIGS. 43A and 43B shows Cas9-mediated gene disruption is associated with protein reduction and high cell viabilities. (43A) TMD8 cells were mock treated or transfected with either of the two PRDM1-targeting RNPs and protein levels were analyzed five days later by western blot. (43B) Viabilities of primary human B cells two days or five days after genome editing.

FIG. 44A-44E shows Cas9-mediated disruption of PRDM1, IRF4, PAX5, or BACH2 alters B cell phenotype. (44A) Experimental workflow of the plasma cell differentiation assay. This workflow includes a three-step differentiation culture that is divided by a B cell activation phase (phase 1), a plasmablast differentiation phase (phase 2) and a plasma cell differentiation phase (phase 3) using the different cocktails of soluble factors and cytokines specified. Cells were transfected with Cas9 RNP after two days of initial activation, indels were assessed in genomic DNA on day 5, and phenotypes and IgM/IgG secretion were quantified on day 11. (44B) Schematic diagram of the roles of transcription factors PRDM1, IRF4, PAX5, and BACH2 as established by murine studies. (44C-44E) Primary B cells were activated and either mock treated, or transfected with Cas9 RNP targeting CCR5, PRDM1, IRF4, PAX5, or BACH2 and were subsequently differentiated in vitro. (44C) Top: representative flow plots showing CD19 and CD38 expression in live singlets and bar graph showing mean percentages of CD19lowCD38high plasmablasts 11 days post transfection. Bottom: representative flow plots showing CD27 and CD138 expression and bar graph showing percentage of CD27+CD138+ plasma cells 11 days post transfection (n=5, three donors). (44D) Genomic DNA was isolated five days after RNP transfection for allelic indel analysis by sequencing (n=5). (44E) Amounts of IgM (left) and IgG (right) in each culture as measured by ELISA (n=4, three donors). All bar graphs show means±SEM. n represents the number of independent experiments. Dotted lines mark mock levels. One-way ANOVA with the Sidak correction for multiple comparisons is used; *p<0.05; **p<0.01; ***p<0.001; ns, not significant. Statistical comparisons were made compared to mock. IFN-α, interferon α; PB, plasmablast; PC, plasma cell.

FIG. 45A-45C shows Cas9 RNPs targeting CCR5, PRDM1, IRF4, PAX5, and BACH2 lead to high levels of gene disruption in primary human B cells. (FIG. 45A-45C) Primary B cells were activated for two days and either mock treated, or transfected with Cas9 RNP targeting CCR5, PRDM1, IRF4, PAX5, or BACH2 and were subsequently differentiated in vitro. (45A) IgG and IgM levels were quantified on day 11 by ELISA and normalized to cell numbers. IgG/IgM mass ratios were calculated and shown in bar graph (n=4). (45B) Cells were immunophenotyped on day 11 and gated on live, singlet, CD19lowCD38high plasmablasts. Left: representative CD19 histograms. Right: mean fluorescence intensities of CD19 (n=5). One-way ANOVA with the Sidak correction for multiple comparisons; *p<0.05; ***p<0.001; ns, not significant. Statistical comparisons were made compared to mock. As shown in FIG. 45 B from top.

FIG. 46A-46B shows AAV-transduced B cells are marked by rapid recovery and comparable viabilities to mock at experimental endpoint. (46A) Primary B cells were activated for two days in culture and transduced with scAAV GFP of various serotypes at 25,000 MOI. Bar graph shows viabilities two days after transduction (n=3). (46B) Primary B cells were activated for two days and gene-edited using PRDM1-targeting RNP and AAV PRDM1-GFP at different MOIs. Bar graph shows cell viabilities on day 2 and 11 in mock, BFP-no-HA control or PRDM1-GFP edited B cell cultures (n=4). All values are means SEM. As shown in 46B in the bar graphs are Day 2 and Day 11 consecutively.

FIG. 47A-47D shows HDR-mediated GFP addition at the PRDM1 locus using Cas9 RNP and an AAV6 donor template leads to progressively increased GFP expression in a dose-dependent manner. (47A) B cells were activated for two days and were either mock treated or transduced with the GFP-expressing scAAV packaged using a comprehensive panel of AAV serotypes (1, 2, 2.5, 5, 6, 8, 9 or D-J) at a multiplicity of infection (MOI) of 25,000. Shown are the percentages of GFP+ cells (left) and mean fluorescence intensities (right) (n=3, three donors). (47B) Schematic of wild type PRDM1 locus, PRDM1g-2 target location and an AAV GFP expression cassette with 400 bp flanking PRDM1 homology arms (AAV PRDM1-GFP). The 3-base PAM sequence is deleted from the AAV template homology sequence. (47C, 47D) B cells were activated for two days and were either mock treated or transfected with Cas9 RNP-PRDM1g-2 with or without AAV transduction. Cells were subsequently cultured under the same activating condition for 11 days. (47C) Top: representative flow plots showing BFP and GFP expressions on day 2 and day 11 after genome editing. The boxed area of the bottom row second panel highlights the GFP+ population in the gene-edited B cells. Bottom: bar graph showing percentages of GFP+ cells on day 2 and day 11 after gene-editing (n=4, four donors). All bar graphs show means±SEM. n represents the number of independent experiments. One-way ANOVA with the Sidak correction for multiple comparisons is used; *p<0.05; ***p<0.001; ns, not significant. MFI, mean fluorescence intensity. PAM, protospacer adjacent motif, WT, wildtype; HA, homology arm; pA, SV40 poly-adenylation signal.

FIG. 48A-48B shows targeted addition of GFP at the PRDM1 locus using RNP and AAV donor templates containing either 400 bp or 1.0 kb homology arms leads to stable GFP expression. (48A) Schematic of AAV PRDM1-GFP cassettes flanked by either 400 bp (top) or 1.0 kb (bottom) homology arms. The template on the top is identical to the one shown in FIG. 3. Both templates leave out the 3 bp PAM sequence. (48B) Primary B cells were activated for two days and gene-edited. Bar graph shows percentages of GFP+ cells on day 2 and day 11 after genome editing (n=3). All values are means±SEM. Unpaired two-tailed t-test; ns, not significant. HA, homology arm; ns, not significant.

FIG. 49A to 49E shows HDR-mediated integration of BAFF coding sequence at the CCR5 locus results in persistent BAFF secretion by gene-edited plasma cells and increases plasma cell differentiation and viability. (49A) Schematic of wild type CCR5 locus, CCR5g target location, an AAV construct that co-expresses GFP and BAFF via T2A linkage (AAV CCR5-GFP-BAFF) and a BAFF-expressing AAV construct (AAV CCR5-BAFF) with identical 800 bp flanking CCR5 homology arms. (49B) B cells were gene-edited after two days of in vitro activation, and were subsequently differentiated into plasma cells using the three-step culture system. Left: representative flow plots showing GFP expression on day 2 and day 11 post gene-editing in mock, CCR5-GFP-BAFF transduced cells with or without Cas9 RNP. Right: bar graph summarizing percentages of GFP+ cells on day 2 and day 11 post gene-editing (n=4, three donors). (49C) B cells were mock electroporated or transduced with AAV CCR5-GFP-BAFF or AAV CCR5-BAFF, with or without Cas9 RNP. Bar graph shows BAFF production as measured by ELISA at day 11 (n=4, three donors). (49D) Left: cells were counted at day 5 and day 11 post gene-editing. Bar graph shows fold changes in cell numbers. Right: viabilities at day 11 by flow cytometry. (n=5, four donors) (49E) Left: representative flow plots showing CD19 and CD38 expression at day 11 after genome editing using the AAV CCR5-BAFF vector. Right: bar graph summarizing percentages of CD19lowCD38high plasmablasts/plasma cells at day 11 (n=5, four donors). Both AAV CCR5-GFP-BAFF and CCR5-BAFF were added at 20,000 MOI. All bar graphs show means±SEM. N represents the number of independent experiments. One-way ANOVA was used with the Sidak correction for multiple comparisons; **p<0.01; ***p<0.001. PAM, protospacer adjacent motif, IL2ss, IL2 signal sequence; WT, wildtype; HA, homology arm; pA, SV40 poly-adenylation signal.

FIG. 50 shows that using AAV without CCR5 homology leads to minimal fluorochrome expression. B cells were either mock electroporated or co-treated with CCR5-targeting RNP and an AAV BFP-noHA control after two days of in vitro activation, and were subsequently differentiated into plasma cells using the three-step culture system. Representative flow plots on day 2 and day 11 (left) and bar graph showing mean percentages of BFP+ cells±SEM (n=4).

FIG. 51A-51D shows HDR-mediated integration of FIX coding sequence at the CCR5 locus leads to high levels of FIX secretion by gene-edited plasma cells ex vivo. (51A) Schematic of wild type CCR5 locus, CCR5g target location, and a FIX-expressing AAV construct with 800 bp flanking CCR5 homology arms (AAV CCR5-FIX). (51B-51D) Primary B cells were gene-edited using the AAV CCR5-FIX donor template and CCR5-targeting RNP (CCR5g RNP) with or without PAX5-targeting RNP (PAX5g RNP). B cells were subsequently differentiated in vitro. (51B) Frequency of on-target FIX integration in total alleles on day 11 after genome editing as assessed by digital droplet PCR. (51C) CD19 and CD38 expression on day 11. Left: representative flow plots; right: mean percentages of CD19lowCD38high plasmablasts (n=2). (51D) FIX production at day 11 after genome editing as measured by ELISA (n=3, two donors). AAV was added at 20,000 MOI. Bar graphs show means±SEM. n represents the number of independent experiments. One-way ANOVA with the Sidak correction for multiple comparisons were used; **p<0.01; ***p<0.001. PAM, protospacer adjacent motif, WPRE3, a shortened woodchuck hepatitis virus posttranscriptional regulatory element 55; WT, wildtype; HA, homology arm; pA, SV40 poly-adenylation signal.

FIG. 52A to 52C shows Outline of flow cytometry gating strategy. (52A-52B) Activated B cells were analyzed by flow cytometry. (52A) Preliminary gates for live cells (left) and singlets (middle) were used to define a viable cell population in unstained B cells before measuring fluorescence (GFP shown on right). (52B) Cells were stained with DAPI to mark dead cells. Viability levels were confirmed by quantifying percentages of DAPI-cells. (52C) Demonstration of general flow cytometry gating strategy for analyzing B cell immunophenotypes. B cells were stained with fluorochrome-conjugated anti-CD27, CD138, CD19, CD38 and live/dead stain-Alexa Fluor 350. Live singlets were defined using FSC/SSC, FSC-W/FSC-H and AF350-gates, and the resulting cell population was analyzed for expression of CD markers. Positive and negative populations were separated based on unstained controls. DAPI, 4,6-diamidino-2-phenylindole.

FIG. 53A-53D shows a description of library preparation and molecular analysis of gene disruption and ssODN editing studies. (53A) Genomic DNA flanking the cut site was initially amplified using sequence specific primers to introduce molecular adapters (orange bars). After bead-based purification to remove primers and enzyme, these sequences were amplified with primers containing molecular indices unique to each experiment (red bar) and cluster-generating sequences (purple bars). The samples were purified, pooled and analyzed using a NextSeq® 500 Mid-kit. Finally, the data were de-multiplexed, indel and HDR frequencies were quantified using the CRISPResso software package. (53B) An example from one replicate showing indel frequency and read-depth for the IRF4 guide. (53C) A histogram showing the percent insertion, deletion and substitutions plotted relative to the guide cut site (dotted line) in the 150 bp read sequence. (53D) The number of sequences corresponding to each indel size quantified in this experiment (negative and positive numbers represent deletions and insertions, respectively).

FIGS. 54A-54C depict gene-edited, BAFF-expressing plasma cells stably secrete BAFF and human immunoglobulins in NSG mice. FIG. 54A depicts an experimental layout of NSG mouse transplant. Gene-edited B cells were generated as before after two days of in vitro B cell activation and were subsequently differentiated into plasma cells using the three-step culture system. 11 days post genome editing, cells were delivered intravenously into NSG mice (conditioned with Busulfan a day before transplant) at 10 million cells/animal. Blood samples were collected at day 10 and, finally, at day 21 when mice were sacrificed. FIG. 54B and FIG. 54C depict graphs from an experiment in which blood serum proteins were quantified by ELISA at day 10 and day 21 (n=3). Shown are serum BAFF levels (FIG. 54B) and serum human IgM and IgG levels (FIG. 54C). All graphs represent means±SEM. n represents the number of mice per group. One-way ANOVA with the Sidak correction for multiple comparisons between groups was used, and paired two-tailed t-test for comparisons between two time points (day 10 vs 21); *p<0.05; **p<0.01; ***p<0.001. NSG, NOD/SCID/gamma-c null; PCs, plasma cells; IV, intravenous.

FIG. 55 shows data that demonstrates that in vivo delivery of human APRIL or IL6 enables increased long-term secretion by edited long-lived plasma cells. Primary human B cells were expanded in culture and edited as described in the alternatives herein. Following differentiation into plasma cells, 10 million were injected into NSG mice. Human BAFF was quantified using ELISA. Each dot represents data from an individual recipient mouse at the indicated time point.

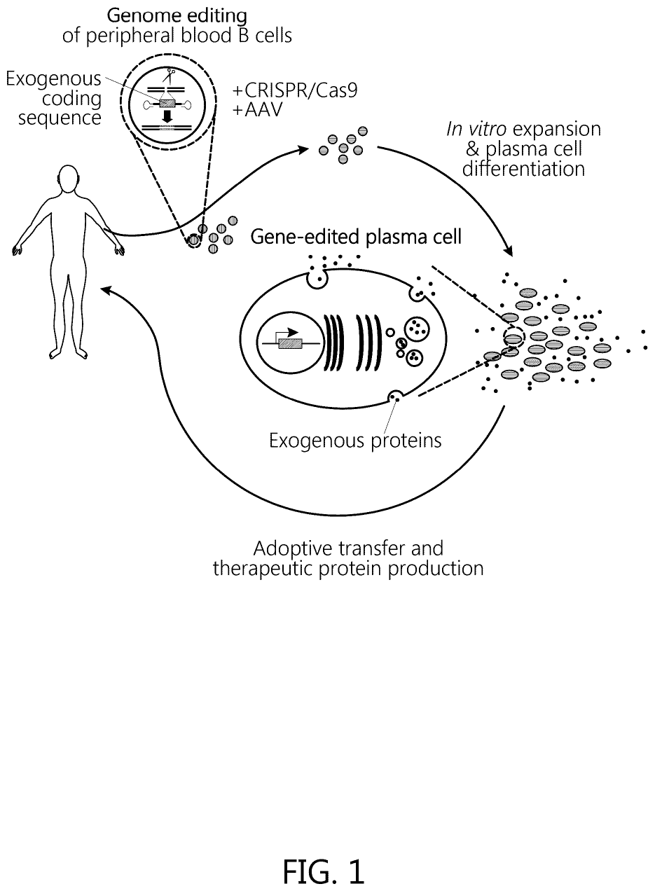

### DEFINITIONS

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

“Nucleic acid” or “nucleic acid molecule” have their plain and ordinary meaning when read in light of the specification, and may include but is not limited to, for example, polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some alternatives, a nucleic acid sequence encoding a protein is provided. In some alternatives, the nucleic acid is RNA or DNA.

“Macromolecule” has its plain and ordinary meaning when read in light of the specification, and may include but is not limited to, for example, a large molecule, such as a protein. The most common macromolecules in biochemistry can include nucleic acids, proteins, peptides, carbohydrates and macrocycles, for example. In the alternatives herein, plasma cells or plasma cell precursors are provided, wherein the plasma cells or plasma cell precursors express a macromolecule, such as a protein, protein mimetic, or a peptide. In some alternatives, the macromolecule is a prodrug.

“Prodrug” has its plain and ordinary meaning when read in light of the specification, and may include but is not limited to, for example, an inactive molecule, or macromolecule that is metabolized into a pharmacologically active drug. After administration, the prodrug is metabolized into a pharmacologically active drug. Prodrugs can be classified into two types, Type I prodrugs may be bioactivated inside the cells (intracellularly). Examples of these are anti-viral nucleoside analogs that must be phosphorylated and the lipid-lowering statins. Type II prodrugs are bioactivated outside cells (extracellularly), especially in digestive fluids or in the body's circulatory system, particularly in the blood. Examples of Type II prodrugs are salicin (described above) and certain antibody-, gene- or virus-directed enzyme prodrugs used in chemotherapy or immunotherapy.

“Proenzyme” has its plain and ordinary meaning when read in light of the specification, and may include but is not limited to, for example, a precursor of an enzyme. In some alternatives, the macromolecule is a protein, such as a proenzyme. In some alternatives, the proenzyme is Factor XI. In some alternatives, the proenzyme is from the family of the coagulation system. In some alternatives the proenzyme comprises fibrinogen.

“Protein” has its plain and ordinary meaning when read in light of the specification, and may include but is not limited to, for example, large macromolecules that comprise long chains of amino acid residues. Protein, polypeptide, and peptide can be ambiguous terms and can overlap in meaning. The term “protein” can refer to the complete biological molecule in a stable conformation, whereas “peptide” can be reserved for short amino acid oligomers that can lack a stable three-dimensional structure. The boundary between the two terms is not well defined and the number of amino acids can be close to about 20-30 residues for a peptide. The term “polypeptide” refers to a single linear chain of amino acids, usually regardless of length. Without being limiting proteins can be a serum protein, glycoprotein, lipoprotein, enzyme, nucleoprotein, structural protein, antibody, or prodrug, for example.

“B-cell activating factor” (BAFF), can also be referred to as “tumor necrosis factor ligand superfamily member 13B.” BAFF has its plain and ordinary meaning when read in light of the specification, and may include but is not limited to, for example, a protein that is encoded by the TNFSF 13B gene. BAFF is a cytokine that belongs to the tumor necrosis factor (TNF) ligand family. In some alternatives herein, a B cell or plasma cell is provided, wherein the B cell or plasma cell expresses BAFF. BAFF has been shown to interact with and activate noncanonical NF-xB signaling pathways. This interaction triggers signals essential for the formation and maintenance of B cell, thus it is important for a B-cell survival. In some alternatives, the cells are manufactured to secrete BAFF to improve or enhance B-cell survival.

MHC class II molecules, have their plain and ordinary meaning when read in light of the specification, and may include but is not limited to, for example, a class of major histocompatibility complex (MHC) molecules normally found only on antigen-presenting cells such as dendritic cells, mononuclear phagocytes, some endothelial cells, thymic epithelial cells, and B cells. These cells are important in initiating immune responses.

A protein, polypeptide or peptide can also be found with a functional group or an additional moiety for functional purposes such as an enzyme co-factor, for example. Without being limiting a protein, polypeptide or peptide can comprise a hydrophobic group for membrane localization. Hydrophobic groups can be added by myristoylation, palmityolation, isoprenylation, prenylation, farnesyltion, gerangylgeranylation or glypiation, for example.

Cofactors are another functional group, which are used for enhanced enzymatic activity. Without being limiting, cofactors can include lipolyation, Flavin moiety, heme C attachment, phophopantetheinylation, retinylidene formations.

Proteins and peptides can also comprise modified amino acids or non-natural amino acids. In some alternatives, the macromolecule comprises a protein mimetic. In some alternatives, the macromolecule is a prodrug.

In some alternatives herein, a method for of making plasma cells or plasma cell precursors that expresses a molecule, such as a protein, peptide or macromolecule is provided, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the molecule is a prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites. In some alternatives, the molecule is a macromolecule, such as a protein or an enzyme. In some alternatives, the protein or enzyme is for protein replacement, enzyme replacement and rescue of enzyme or protein deficiencies. In some alternatives, the macromolecule comprises Factor VIII, Factor IX, ADAMTS13, LIPA, SERPING1, SERPINA1, GLA, and/or ALPL. In some alternatives, the macromolecule is a prodrug.

“Cytokines” have their plain and ordinary meaning when read in light of the specification, and may include but is not limited to, for example, a group of proteins that function in cell signaling. For example cytokines can be involved in autocrine signaling, paracrine signaling and endocrine signaling as immunomodulating agents. Cytokines are important in health and disease, for example, playing arole in host responses to infection, immune responses, inflammation, trauma, sepsis, cancer, and reproduction. In some alternatives herein, a method for of making plasma cells or plasma cell precursors that expresses a molecule, such as a protein, peptide or macromolecule is provided, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the molecule is a macromolecule, such as a cytokine, cytokine receptor, complement protein or other inhibitory protein including, but not limited to: Ill receptor antagonist for treatment or amelioration of periodic fever/autoinflammatory syndromes; complement inhibitory proteins (including Factor H, Factor I) for treatment or inhibition of atypical hemolytic uremic syndrome/membranoproliferative glomerulonephritis; and/or C1 inhibitor for hereditary angioedema.

“Anti-fibrotic molecule” has its plain and ordinary meaning when read in light of the specification, and may include but is not limited to, for example, a molecule that is used in the treatment of fibrosis, or excess fibrous connective tissue in an organ or tissue in a reparative or reactive process. Fibrosis can include pulmonary fibrosis, cystic fibrosis, Idiopathic pulmonary fibrosis, liver cirrhosis, atrial fibrosis, endomyocardial fibrosis, myocardial infarction, glial scars of the brain, arterial stiffness, Arthrofibrosis (knee, shoulder, other joints), Crohn's Disease (intestine), Dupuytren's contracture (hands,fingers), Keloid (skin), Mediastinal fibrosis (soft tissue of the mediastinum), Myelofibrosis (bone marrow), Peyronie's disease, Nephrogenic systemic fibrosis (skin), Progressive massive fibrosis (lungs), Retroperitoneal fibrosis (soft tissue of the retroperitoneum), Scleroderma/systemic sclerosis (skin, lungs) and/or some forms of adhesive capsulitis (shoulder). Without being limiting, anti-fibrotic molecules can include but is not limited to SCGB1A1 for the treatment of pulmonary fibrosis.

“Antibodies” also known as “immunoglobulins” have their plain and ordinary meaning when read in light of the specification, and may include but is not limited to, for example, large Y-shaped proteins that are secreted by plasma cells to neutralize pathogens. In some alternatives herein, a method for of making plasma cells or plasma cell precursors that expresses a molecule, such as a protein, peptide or macromolecule is provided, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the molecule is an antibody, or a portion thereof. In some alternatives herein, the plasma cell or plasma cell precursor expresses an antibody.

“Coding for” or “encoding” are used herein, has itsplain and ordinary meaning when read in light of the specification, and may include but is not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

“Vector,” “Expression vector” or “construct” have their plain and ordinary meaning when read in light of the specification, and may include but is not limited to, for example, a nucleic acid used to introduce heterologous nucleic acids into a cell that has regulatory elements to provide expression of the heterologous nucleic acids in the cell.

“B cells” or “B lymphocytes” have their plain and ordinary meaning when read in light of the specification, and may include but is not limited to, for example, a white blood cell type of the lymphocyte subtype. B cells are unlike lymphocytes such as T cells and natural killer cells, as B cells express B cell receptors on their cell membrane. The B cell receptors allow the B cell to bind a specific antigen, which will initiate an antibody response. B cells develop from hematopoietic stem cells. As described herein, B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof depending on the context.

B cell precursors include the cells from which the B cells are derived. Like T cells, B cells are lymphatic cells that are originated form the bone marrow, where they can reside until they are mature. The B cells, as described in the alternatives herein, include stem cells, early pro-B cells, late pro-B cells, large pre-B cells, small pre-B cells, immature B cells, T1 B cells, T2 B cells, marginal zone B cells, mature B cells, naïve B cells, plasmablasts (short-lived) cells, GC B cells, memory B cells, plasmablast cells and/or long lived plasma cells. In some alternatives of the plasma cell for expressing a molecule such as a macromolecule, protein or a peptide, the plasma cell is derived from a B cell. In some alternatives, the B cell is a memory B cell. In some alternatives, the B cell is a stem cell, early pro-B cell, late pro-B cell, large pre-B cell, small pre-B cell, immature B cell, T1 B cell, T2 B cell, marginal zone B cell, mature B cell, naïve B cell, plasmablasts (short-lived) cell, GC B cell, memory B cell, plasmablast cell or a long lived plasma cell. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the macromolecule is a prodrug.

“Memory B cells” have their plain and ordinary meaning when read in light of the specification, and may include but is not limited to, for example, the B cell sub-types that are formed within germinal centers following primary infection and are important in generating an accelerated and more robust antibody-mediated immune response in the case of re-infection. The B lymphocytes form the memory cells that can remember the same pathogen for future antibody production during future infections. In some alternatives of the plasma cell for expressing a molecule such as a macromolecule, protein or a peptide, the plasma cell is derived from a B cell. In some alternatives, the B cell is a memory B cell. In some alternatives, the macromolecule is a prodrug.

“Naïve B cell” has itsplain and ordinary meaning when read in light of the specification, and may include but is not limited to, for example, a B cell that has not yet been exposed to an antigen. Once exposed to an antigen, the naïve B cell becomes a memory B cell. In some alternatives of the plasma cell for expressing a molecule such as a macromolecule, protein or a peptide, the plasma cell is derived from a B cell. In some alternatives, the B cell is a memory B cell. In some alternatives, the macromolecule is a prodrug. “Peripheral blood mononuclear cells” (PBNC) as described herein are peripheral blood cells having a round nucleus. These cells consist of lymphocytes (T cells, B cells, NK cells) and monocytes, whereas erythrocytes and platelets have no nuclei, and neutrophils, basophils, and eosinophils have multi-lobed nuclei. In the alternatives described herein, the B cells are subject derived or are allogeneic peripheral blood mononuclear cells. In some alternatives, the B cells are blood-derived human B cells.

“Plasma cells” as described herein, are also called plasma B cells, plasmocytes, plasmacytes, or effector B cells. Plasma cells have their plain and ordinary meaning when read in light of the specification, and may include but is not limited to, for example, white blood cells that secrete antibodies and are transported by the blood plasma and the lymphatic system.

“Plasma cell precursor” can begin as an immature plasma cell. The most immature blood cell of the plasma cell lineage is called the plasmablast which can differentiate into a mature fully differentiated plasma cells. Plasmablasts can secrete more antibodies than a B cell, but less than a plasma cell. In some alternatives, a method of making a plasma cell that expresses a molecule is provided. In some alternatives, the plasma cell is a plasma cell precursor. In some alternatives, the plasma cell precursor is a plasmablast.

Cell isolation,” also referred to as “isolating of cells,” have their plain and ordinary meaning when read in light of the specification, and may include but is not limited to, for example, a cell separation technique. Such techniques are important in immunology and the techniques can be appreciated by those of skill in the art. Isolation or the separation of the cells can be performed, for example, by the use of antibodies against differentially expressed cell-surface molecules of targets. Without being limiting, B cells can be isolated by using a commercially available kit. Without being limiting, there are commercially available kits for B cell isolation by Miltenyi Biotec®, EasySep® Human B Cell Isolation Kit by StemCell® Technologies, and many others. In some alternatives herein, a method of making plasma cells or plasma cell precursors that express a molecule, such as macromolecule is provided, wherein the method comprises a step for isolating B cells. In some alternatives, the macromolecule is a protein, a protein mimetic or a peptide. In some alternatives, a method of making plasma cells or plasma cell precursors that express a molecule, such as a macromolecule is provided, the method comprising isolating B cells, developing the B cells, performing a first round of genome editing of the B cells for protein expression in absence of viral integration, expanding the B cells; and differentiating the B cells, optionally, after step (c) or (d), thereby producing plasma cells that express the molecule.

“Development” of a cell describes a cells differentiation to a cell type. For example, development into a B cell can occur in several stages, wherein each stage is marked by various gene expression patterns and immunoglobulin H chain and L chain gene loci arrangement. For example the B cells undergo VJ recombination as they develop. B cells develop from hematopoietic stem cells that originate from bone marrow. In some alternatives herein, a method of making plasma cells or plasma cell precursors that express a molecule such as a macromolecule is provided, wherein the method comprises a step for developing the B cells. In some alternatives, the cells are memory B cells or naïve B cells. In some alternatives, the macromolecule is a prodrug.

B cells undergo two types of selection while developing in the bone marrow to ensure proper development. For example, positive selection occurs through antigen independent signaling. Negative selection occurs through the binding of self-antigen with the B cell receptor.

“Genome editing” has itsplain and ordinary meaning when read in light of the specification, and may include but is not limited to, for example, a process that include methods for genetic engineering in which DNA is inserted, deleted or replaced in the genome of a living organism. Editing a gene is also known as gene editing. In some alternatives described herein, a method of making plasma cells or plasma cell precursors that express a molecule, such as a macromolecule is provided, in which B cells or B cell precursors are subjected to at least one round of genome editing. Methods of genome editing can include, but is not limited to nucleic acid being inserted, deleted or replaced in the genome of a cell. In some alternatives, a nuclease is used to achieve this process. In some alternatives, the nuclease is engineered. In some alternatives, the methods include inducing double strand breaks that are repaired through nonhomologous end-joining (NHEJ) or homologous recombination (HR). In some alternatives, the step of genome editing is performed by introduction of a single stranded nucleic acid. In some alternatives, the at least one round of genome editing further comprises cycling the B-cells for homologous recombination of the single stranded DNA oligonucleotides or recombinant adeno-associated virus into the candidate genetic loci. In some alternatives, the genome editing of the B cells for protein expression is performed in the absence of viral integration. In some alternatives, a second round of genome editing is performed to excise a region. In some alternatives, a third round of genome editing is performed to result in expression of a drug activatable growth enhancer. In some alternatives herein, the genome editing is performed by nonpathogenic AAV mediated editing by direct homolougous recombination.

Genome editing can also employ the use of RNA and protein based transfection. For example the CRISPR/Cas system can be modified to edit genomes. This technique requires the delivery of the Cas nuclease complexed with a synthetic guide RNA (gRNA) into a cell, thus the cell's genome can be cut at a specific location and allow existing genes to be removed and/or add new ones. Thus, CRISPR/Cas and related programmable endonuclease systems are rapidly becoming significant genome editing tools of the biomedical research laboratory, with their application for gene disruption and/or gene targeting as demonstrated in a variety of cultured cell and model organism systems. In some alternatives, of the CRISPR/Cas system described herein, the Cas nuclease comprises Cas1, Cas2, Cas3, Cas4, Cas5, Cas6, Cas7, Cas8 or Cas9.

The basic components of CRISPR/Cas system comprise a target gene, a protospacer adjacent motif (PAM), a guide RNA, Cas endonuclease. An important aspect of applying CRISPR/Cas for genome editing is the need for a system to deliver the guide RNAs efficiently to a wide variety of cell types. This could, for example, involve delivery of an in vitro generated guide RNA as a nucleic acid (the guide RNA generated by in vitro transcription or chemical synthesis). In some alternatives, the nucleic acid could be rendered nuclease resistant by incorporation of modified bases.

The CRISPR-Cas system falls into two classes. The Class 1 system has a complex of multiple Cas proteins for the degradation of foreign nucleic acids. The Class 2 system has a single large Cas protein for a same purpose for the degradation of foreign nucleic acids. There are a 93 cas genes that are grouped into 35 families. 11 of the 35 families from a cas core which includes the protein families CAS1 to CAS9. As described herein, Cas comprises Cas1, Cas2, Cas3, Cas4, Cas5, Cas6, Cas7, Cas8 or Cas9.

Gene editing may also be performed by a novel non-nuclease based gene editing platform. A novel family of AAVs were previously isolated from human hematopoietic stem cells. These nonpathogenic AAVs are naturally present in healthy individuals and may possess unique gene editing and gene transfer properties. This technique is also described as AAV mediated editing by direct homolougous recombination (AmENDR™). This process is homolougous recombination by a natural biological mechanism that is used by cells to ensure highly precise DNA repair.

AAV mediated editing by direct homolougous recombination is initiated by design of homology sequence “arms” that are specific to a region of the genome and results in a permanent correction in the DNA when administered to cells. In some alternatives herein, the gene editing is performed by nonpathogenic AAV mediated editing by direct homolougous recombination. The identification of novel AAV genomes are described in Smith et al. (Mol Ther. 2014 September; 22(9): 1625-1634; incorporated by reference in its entirety herein). The novel AAVs described by Smith et al., represents a new class of genetic vector for the manipulation of HSC genomes. Furthermore, these vectors may greatly expand the ability to deliver genes to targeted tissues and cells including cells that are refractory to gene transfer which circumventing prevalent preexisting immunity to AAV2. In some alternatives, the gene editing is performed by nonpathogenic AAVs naturally present in hematopoietic cells, wherein the editing is performed by AAV mediated editing by direct homolougous recombination using the nonpathogenic AAVs as described in Smith et al.

“Engineered nucleases” have their plain and ordinary meaning when read in light of the specification, and may include but is not limited to, for example, enzymes that are engineered to be hybrid enzymes which can be used to specifically recognize a DNA sequence and efficiently edit the genome by the introduction of double-strand breaks. Without being limiting, there are four families of engineered nucleases are meganucleases, zinc finger nucleases (ZFN), transcription activator like effector-based nucleases (TALEN), and the CRISPR-Cas system.

“Meganucleases” have their plain and ordinary meaning when read in light of the specification, and may include but is not limited to, for example, endodeoxyribonucleases characterized by a large recognition site (double-stranded DNA sequences of 12 to 40 base pairs). In some alternative methods for making a plasma cell or plasma cell precursor that expresses a molecule such as a macromolecule, the method comprises: (a) isolating B cells, (b) developing the B cells, (c) performing a first round of genome editing of the B cells for protein expression in absence of viral integration, (d) expanding the B cells, and (e) differentiating the B cells, optionally, after step (c) or (d), thereby producing plasma cells that express a protein. In some alternatives, the first round of genome editing is performed by an RNA and protein based transfection. In some alternatives, the nuclease is a meganuclease.

“Zinc finger nucleases (ZFN)” have their plain and ordinary meaning when read in light of the specification, and may include but is not limited to, for example, engineered restriction enzymes that are generated by fusing a zinc finger DNA-binding domain to a DNA-cleavage domain. Zinc finger domains can be engineered to target specific desired DNA sequences and this enables zinc-finger nucleases to target unique sequences within complex genomes. In some alternative methods for making a plasma cell that expresses a molecule, such as a macromolecule, the method comprises: (a) isolating B cells, (b) developing the B cells, (c) performing a first round of genome editing of the B cells for protein expression in absence of viral integration, (d) expanding the B cells, and (e) differentiating the B cells, optionally, after step (c) or (d), thereby producing plasma cells that express a protein. In some alternatives, the first round of genome editing is performed by an RNA and protein based transfection. In some alternatives, the nuclease is a zinc finger nuclease.

“Transcription activator-like effector nucleases,” (TALEN), have their plain and ordinary meaning when read in light of the specification, and may include but is not limited to, for example, restriction enzymes that can be engineered to cut specific sequences or sites in DNA. They are made by fusing a TAL effector DNA-binding domain to a DNA cleavage domain (a nuclease which cuts DNA strands). Transcription activator-like effectors (TALEs) can be engineered to bind to a desired DNA sequence, so when combined with a nuclease, the DNA can be cut at specific locations. Thus, the restriction enzymes can be introduced into cells, for use in genome editing or for genome editing in situ, a technique known as genome editing with engineered nucleases. The use of TALEN is known to those of skill in the art. In some alternatives described herein, a method of making plasma cells or plasma cell precursors that express a molecule, such as a macromolecule is provided, in which B cells or B cell precursors are subjected to at least one round of genome editing. Methods of genome editing can include, but is not limited to nucleic acid being inserted, deleted or replaced in the genome of a cell. In some alternatives, a nuclease is used to achieve this process. In some alternatives, the nuclease is engineered. In some alternatives, the methods include inducing double strand breaks that are repaired through nonhomologous end-joining (NHEJ) or homologous recombination (HR). In some alternatives, the method includes a first round of genome editing or genome editing. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary.

“Expansion” as described herein, refers to steps to increase the yield of the cells. In order for a group of cells to be useful or beneficial, the expansion of cells following isolation or extraction is necessary. The ability to expand B cell populations ex vivo is valuable for downstream applications including high throughput B cell assays and ex vivo differentiation of the cells. For example steps for increasing the number of B cells or gene edited B cells can include but are not limited to methods for introducing genetic modifications that artificially induce non-transformative expansion of gene edited B cells. Furthermore, there are also commercially available kits that comprise reagents for the growth and expansion of B cells. These methods and commercially available kits for B cell expansion can be appreciated by those of skill in the art. In some alternatives herein, the gene editing is performed by nonpathogenic AAV mediated editing by direct homolougous recombination.

For “rapid B cell expansion,” as described herein, edits to a B cell are introduced two days after activating and expanding for five additional days. It is expected that the cells will be expanded 50-fold.

“Differentiation” as described herein, refers to a cell changing from one cell type into another. Without being limiting, B cells can be differentiated based on their exposure to T cell-derived cytokines bound by B cell cytokine receptors. For example, CD40L can serve as a necessary stimulatory factor for B cell activation by binding the B cell surface receptor CD40, which can also affect differentiation. In the alternative methods described herein, the B cell is differentiated in a three step culture system comprising activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα.

“Somatic hypermutation” (or SHM) has itsplain and ordinary meaning when read in light of the specification, and may include but is not limited to, for example, a cellular mechanism by which the immune system adapts to the new foreign elements that confront it (e.g. microbes), as seen during class switching. SHM diversifies B cell receptors used to recognize foreign elements (antigens) and allows the immune system to adapt its response to new threats during the lifetime of an organism. Somatic hypermutation involves a programmed process of mutation affecting the variable regions of immunoglobulin genes. Without being limiting, a cell may be stimulated to divide or proliferate after antigen recognition. During proliferation, the B cell receptor can undergo a high rate of somatic mutation that is at least 10⁵ to 10⁶ greater than the normal rate of mutation across the cellular genome. “Activation-induced cytidine deaminase,” (AICDA and also known as AID), as described herein as AID, is a 24 kDa enzyme which in humans is encoded by the AICDA gene. AID creates mutations in DNA by deamination of cytosine base, which turns it into uracil (which is recognized as a thymine). Through an unknown mechanism, AID changes a C:G base pair into a U:G mismatch. The cell's DNA replication machinery recognizes the U as a T, and hence C:G is converted to a T:A base pair. During germinal center development of B lymphocytes, AID also generates other types of mutations, such as C:G to A:T. In the alternative methods described herein, the method of making plasma cells or plasma cell precursors that express a molecule, such as a macromolecule, further comprises applying methods to prevent somatic hypermutation of an antibody locus in the B cell or B cell precursor. In some alternatives, the method to prevent somatic hypermutation of an antibody locus in the B cell comprises disruption of an AID gene.

“Synthetically engineered protein” has itsplain and ordinary meaning when read in light of the specification, and may include but is not limited to, for example, a protein that is made by protein or expressed from a synthetic gene that is manufactured through the method of genetic engineering. The synthetically engineered protein is usually made through rational protein design and may include unnatural amino acids as well. Furthermore the engineered protein can be transcribed and translated from a gene that is codon optimized for expression in a mammal, such as a human. In some alternatives, a method of making plasma cells or plasma cell precursors that express a molecule, such as a macromolecule is provided. In some alternatives, the macromolecule is a protein, protein mimetic or a peptide. In some alternatives, the protein is a genetically engineered protein. In some alternatives, the genetically engineered protein is encoded by a codon optimized gene. Genetically engineered proteins can also be a fusion or a chimeric protein, which are proteins created through the joining of two or more genes that originally coded for separate proteins or portions of proteins. The fusion proteins can also be made up of specific protein domains from two or more separate proteins. Translation of this fusion gene can result in a single or multiple polypeptides with functional properties derived from each of the original proteins. Recombinant fusion proteins can be created artificially by recombinant DNA technology for use in biological research or therapeutics. Such methods for creating fusion proteins are known to those skilled in the art. Some fusion proteins combine whole peptides and therefore can contain all domains, especially functional domains, of the original proteins. However, other fusion proteins, especially those that are non-naturally occurring, combine only portions of coding sequences and therefore do not maintain the original functions of the parental genes that formed them. In some alternatives, the protein is for prophylactic or therapeutic protection from infection (viral, bacterial, or parasitic) in healthy subject or following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and parasites. In some alternatives, the protein is for protein replacement, enzyme replacement or rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia). In some alternatives, the protein is for immune modulation via expressed cytokines, cytokine receptors, complement proteins or other inhibitory proteins including, but not limited to: Ill receptor antagonist for treatment of periodic fever/autoinflammatory syndromes; complement inhibitory proteins (including Factor H, Factor I) for treatment or amelioration of atypical hemolytic uremic syndrome/membranoproliferative glomerulonephritis; and C1 inhibitor for hereditary angioedema. In some alternatives, the protein expressed is an anti-fibrotic molecule, including, but not limited to SCGB1A1 for the treatment or amelioration of pulmonary fibrosis. In some alternatives, the plasma cell or plasma cell precursor expresses a macromolecule, such as a protein, wherein the protein comprises therapeutic antibodies for autoimmune disorders, autoinflammatory disorders, immune dysregulation and cancer including but not limited to: anti-IL1 monoclonal antibodies for treatment of periodic fever/autoinflammatory syndromes; anti-TNF antibodies for inflammatory arthritis/inflammatory bowel disease, anti-IL33 antibodies for the treatment of asthma and anti-C5 antibodies for treatment of paroxysmal nocturnal hemoglobinuria/atypical HUS. In some alternatives, the plasma cell or plasma cell precursor expresses a macromolecule, such as a protein, wherein the protein is an Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein). In some alternatives, the plasma cell or plasma cell precursor expresses a macromolecule, such as a protein, wherein the protein is a glucose responses element for treatment of diabetic conditions. Synthetically engineered proteins or antibodies that are protective in viral, fungal, parasitic or bacterial infection are also contemplated. Without being limiting the synthetically engineered proteins or antibodies can be specific for HIV, viral pneumonia, or fungal infections.

“Engraftment” as described herein, refers to the expansion of cells and their cell progeny so that they can re-initiate the immune system or become incorporated into the body of the host. As such the cells may be able to grow and reproduce within the recipient. In some alternatives herein, a method of inducing engraftment of a cell that expresses a peptide for immunotherapeutic application, secreted by a B cell in a subject in need in vivo, is provided. In some alternatives, the cell expresses BAFF, APRIL, IL-10, IFN-alpha or IL-6.

A proliferation-inducing ligand (APRIL), as described herein, is also known as tumor necrosis factor ligand superfamily member 13 (TNFSF13), is a protein of the TNF superfamily recognized by the cell surface receptor TAC. In some alternatives herein, a B cell or plasma cell is provided, wherein the B cell or plasma cell expresses APRIL. In some alternatives, the plasma cell secretes IL-10 and/or IL-6. In some alternatives, the cell expresses BAFF, APRIL, IL-10, IFN-alpha or IL-6.

Those skilled in the art will appreciate that gene expression levels are dependent on many factors, such as promoter sequences and regulatory elements. Another factor for maximal protein selection is adaptation of codons of the transcript gene to the typical codon usage of a host. Many synthetic genes can be designed to increase their protein expression level. The design process of codon optimization can be to alter rare codons to codons known to increase maximum protein expression efficiency. In some alternatives, codon selection is described, wherein codon selection is performed by using algorithms that are known to those skilled in the art to create synthetic genetic transcripts optimized for higher levels of transcription and protein yield. Programs containing algorithms for codon optimization are known to those skilled in the art. Programs can include, for example, OptimumGene™, GeneGPS® algorithms, etc. Additionally synthetic codon optimized sequences can be obtained commercially for example from Integrated DNA Technologies and other commercially available DNA sequencing services. In some alternatives herein, the engineered protein is encoded by a gene, wherein the gene is codon optimized for expression in a human cell. In some alternatives, the genetically engineered protein is encoded by a codon optimized gene.

“Proprotein convertase subtilisin/kexin type 9 (PCSK9)” has itsplain and ordinary meaning when read in light of the specification, and may include but is not limited to, for example, an enzyme that is expressed in many tissues and cell types. PCSK9 binds to the receptor for low-density lipoprotein particles (LDL), which typically transport 3,000 to 6,000 fat molecules (including cholesterol) per particle, within extracellular water. If PCSK9 is blocked, more LDLRs are recycled and are present on the surface of cells to remove LDL-particles from the extracellular water. Therefore, blocking PCSK9 can lower blood LDL-particle concentrations. PCSK9 orthologs are found in many species. Agents which block PCSK9 can lower LDL particle concentrations. In some alternatives, a method of making plasma cells or plasma cell precursors that express a molecule, such as a macromolecule is provided. In some alternatives, the macromolecule is a protein, protein mimetic or a peptide. In some alternatives, the protein is a genetically engineered protein. In some alternatives, the genetically engineered protein is encoded by a gene that has been codon optimized for expression in humans. In some alternatives, the genetically engineered protein is specific for PCSK9. In some alternatives, the plasma cell expressing the protein is administered to a subjects suffering from high cholesterol. In some alternatives, subject has at least 200 mg/dL of total cholesterol, wherein total cholesterol includes the levels HDL and LDL cholesterol.

In some alternatives, the plasma cell expressing the protein is administered to a subjects suffering from HIV, CMV or an autoimmune disorder. In some alternatives, the subject is suffering from multiple sclerosis. In some alternatives, the subject is suffering from Crohn's disease. In some alternatives, the protein is an antibody. In some alternatives, the protein comprises Tysabri (natalizumab) antibody or a portion thereof.

“Rituxan®” has its plain and ordinary meaning when read in light of the specification, and may include but is not limited to, for example, a medication sold under the brand name Rituxan® among others, which is a monoclonal antibody specific for CD20 and is used to treat or inhibit certain autoimmune diseases and types of cancer. It is used for non-Hodgkin's lymphoma, chronic lymphocytic leukemia, rheumatoid arthritis, idiopathic thrombocytopenic purpura, and/or pemphigus vulgaris. Rituximab is another name for this anti-CD20 monoclonal antibody. This anti-CD20 antibody is also used to treat rheumatoid arthritis. In RA, this medicine slows the inflammatory process and help reduce joint pain and swelling. This medicine is often used with other cancer or arthritis medications. The success of rituximab likely relates to a number of factors, one of which is that it is a chimeric rather than a murine antibody (Grillo-Lopez, 2000). Rituximab retains the murine CD20-binding Fab regions, but uses a human Fc portion.

“Negative selection of cells” has its plain and ordinary meaning when read in light of the specification, and may include but is not limited to, selecting out cells, for example, in which B-cells and T-cells that recognize MHC molecules bound to peptides of self-origin, or just MHC molecules with high affinity are deleted from the repertoire of immune cells. Negative selection can be performed with a commercial kit, such as a human B cell isolation kit (Miltenyi Biotec®, Auburn, CA).

“Rapamycin” also known as “sirolimus” has its plain and ordinary meaning when read in light of the specification, and may include but is not limited to, a macrolide compound that inhibits activation of T cells and B cells by reducing the production of interleukin-2 (IL-2). Rapamycin-inducible FKBP11 dimers has its plain and ordinary meaning when read in light of the specification, and may include but is not limited to, FKBP (FKBP1A) which will form a dimer in the presence of rapamycin.

The HIPRT1 gene has its plain and ordinary meaning when read in light of the specification, and may include but is not limited to, a gene that provides instructions for producing an enzyme called hypoxanthine phosphoribosyltransferase 1. This enzyme allows cells to recycle purines, a type of building block of DNA and its chemical cousin RNA. Manufacturing purines uses more energy and takes more time than recycling purines, which makes recycling these molecules more efficient. Recycling purines ensures that cells have a plentiful supply of building blocks for the production of DNA and RNA. The process of recycling purines is also known as the purine salvage pathway. More than 200 mutations in the HIPRT1 gene have been identified. These mutations include changes in single DNA building blocks (nucleotides) or insertions or deletions of small amounts of DNA within the gene. These changes result in either nonfunctional or very low-function hypoxanthine phosphoribosyltransferase 1. Under these conditions, uric acid, a waste product of purine breakdown, accumulates in the body and can cause gouty arthritis (arthritis caused by uric acid in the joints), kidney stones, and bladder stones. It is unclear how this enzyme deficiency causes the neurological and certain mutations in the HIPRT1 gene can also cause a condition featuring gouty arthritis called HPRT-related gout, previously known as Kelley-Seegmiller syndrome. Individuals with this condition have lower than normal levels of hypoxanthine phosphoribosyltransferase 1.

“CD138” or “syndecan-1” can be used interchangeably and has its plain and ordinary meaning when read in light of the specification, and may include but is not limited to, a transmembrane (type I) heparan sulfate proteoglycan and is a member of the syndecan proteoglycan family. CD138 s a surface antigen that is expressed at high levels in plasma cells. The CD138 antigen is expressed on normal and malignant plasma cells but not mature B cells. As such, plasma cells may be purified, for example by use of CD138 positive selection and through use of CD138 antibodies attached to beads for cell purification. There are commercial kits that may be used to purify cells that are CD138 positive (e.g. The EasySep® Human CD138 Positive Selection Kit by STEMCELL®, The CD138+ Plasma Cell Isolation Kit by Miltenyl Biotech®). CD138 may be used to purify long lived plasma cells, as this population should engraft better and express more exogenous protein.

### DETAILED DESCRIPTION

As described herein are the methods of making protein producing B cells, and the use of the protein producing B cells for immunotherapies. Additional alternatives can also include the protein producing B cells as well as compositions comprising the protein producing B cells. In some alternatives, the B cells express a macromolecule, such as a protein mimetic or a peptide.

Compositions and methods for inducing tolerance for a peptide secreted by a B cell or inducing engraftment is also provided herein. In some alternatives, these cells express BAFF, APRIL, IL-10, IFN-alpha or IL-6.

Methods for a reproducible system for enrichment and long-term in vitro culture of human B cell progenitors have previously been described by Rawlings et al. 1995, Rawlings et al. 1997, and Fluckinger et al 1998 (“Long-term culture system for selective growth of human B-cell progenitors.” Proc. Natl. Acad. Sci. USA Vol. 92, pp. 1570-1574, February 1995, “Differentiation of human CD34+CD38− cord blood stem cells into B cell progenitors in vitro.” Exp Hematol. 1997 January; 25 (1):66-72; and “In vitro reconstitution of human B-cell ontogeny: from CD34 (+) multipotent progenitors to Ig-secreting cells.” Blood. 1998 Dec. 15; 92(12):4509-20; all references incorporated by reference in their entireties). These types of systems, as previously reported, are important in the production of normal human B-lineage development and includes the production of mature Ig-secreting B cells and were used in studies of normal and abnormal early human B-lymphopoieses. In some systems, the long term in vitro culture system can be initiated with CD34+ or CD34+CD38− umbilical cord blood hematopoietic progenitors that can support normal human B-lineage development and can include the production of mature Ig-secreting B cells (Fluckiger et al. 1998). Although there are methods for isolating and developing B cells for Ig secretion, the development of plasma cells from differentiated B cells that have undergone genome engineering to express a protein has previously not been reported.

As previously reported, there has been genome editing of primary human hematopoietic cells. Co-delivery of designer nuclease mRNA and AAV donor provides a reliable approach for targeted gene modification in primary human hematopoietic cells. Such methods average in about 60% homology-directed repair (HDR) in primary T cells, multiple loci, with multiple experiments and independent donors and averages about 30-50% HDR in adult-mobilized CD34+ cells as shown in Sather et al. (“Efficient modification of CCR5 in primary human hematopoietic cells using a megaTAL nuclease and AAV donor template.” Sci Transl Med. 2015 Sep. 30; 7(307):307ra156; incorporated by reference in its entirety herein). This has also led to efficient delivery of therapeutic gene cassettes into candidate loci, such as HIV therapeutics into CCR5 locus: C46, CD19 CAR, & HIV CAR (Sather et al., Sci Transl Med. 2015 Sep. 30; 7(307); Roman-Ibarra et al., Mol TherNucleic Acids. 2016; 5:e352; Hale et al., Mol Ther Nucleic Acids. 2016, Hale Molec Ther Methods 2016; all incorporated by reference in their entireties herein), safe Harbor or TCRa locus-CARs and other therapeutic cassettes (Hale et al. Molec Ther Methods 2016; incorporated by reference in its entirety herein), CD40L gene in hyper-IgM syndrome to restores CD40L deficient T cell function (Hubbard et al. Blood 2016; incorporated by reference in its entirety herein), and methods to further enhance genome editing using viral helper proteins (Gwiazda et al. Mol Ther. 2016 Sep. 29; 24(9):1570-80; incorporated by reference in its entirety herein). Thus, modification of T cells (or CD34+ HSC) using this co-delivery approach is translatable to a wide range of clinical applications. However this approach was not contemplated for use in B cells.

In T cells, for example, gene targeting was used to disrupt the CCR5 gene. This was done by stable integration of gene cassette at target locus. Homology-directed repair mechanism was used using a megaTAL nuclease and rAAV donor template (Sather and Romano Ibarra et al., Sci Trans Med 2015; incorporated by reference in its entirety herein). Disruption of genes are also used to manufacture HIV protected anti-CD19CAR T cells (Milone et al, 2009 Mol Ther; Porter et al, 2011 NEJM; incorporated by reference in its entirety herein). Thus, successful techniques for genetic editing of B cells, which would also result in cells that have a long life, are much needed. As described herein, are some alternatives for B cell genetic editing, wherein the genome editing is performed by nonpathogenic AAV mediated editing by direct homolougous recombination.

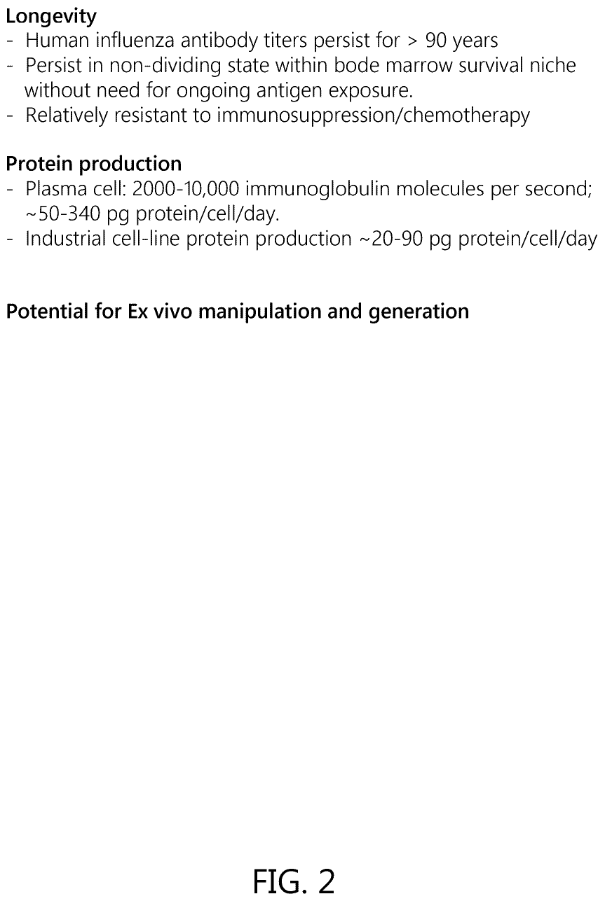

As described herein, are engineered plasma cells as a macromolecule delivery platform. The macromolecule comprises proteins, protein mimetics, or peptides. The potential for the ex vivo manipulation and generation of cells include the increased longevity of the cells and protein production. For example, human influenza antibody titers can persist for over 90 years. These cells can persist in non-dividing state within bone marrow survival niche without need for ongoing antigen exposure and are relatively resistant to immunosuppression/chemotherapy. In regards to protein production, plasma cells can produce about 2000-10,000 immunoglobulin molecules per second, which can translate to about −50-340 pg protein/cell/day. Industrial cell-line protein production can lead to about 20-90 pg protein/cell/day. The alternatives herein have the benefits of using the plasma cells, which are highlighted in FIG. 2.

There are several types of plasma cell-based therapies that are contemplated in the alternative methods described herein. This can include plasma cells that express antibodies that are specific to proteins that are expressed due to infection with a pathogen such as influenza virus, EBV, CMV, HIV, or malaria, for example. Therapeutic proteins are also contemplated. Without being limiting, therapeutic proteins can include proteins to supplement for enzyme deficiencies such as Factor VIII and Factor IX in those suffering from hemophilia, for example. In some alternatives, the therapeutic proteins can bind proteins of the lipid envelope of the influenza virus. In some alternatives, the lipid envelop proteins comprises glycoproteins hemagglutin and neuraminidase. In some alternatives, the therapeutic proteins can bind proteins of the CMV virus. In some alternatives, the proteins of the CMV virus comprises glycoprotein B.

Requirements for edited plasma cell regeneration can include, B cell activation and genome editing, B cell expansion B cell differentiation into long-lived plasma cells (LLPC). LLPC make up about 0.1-1% of all bone-marrow cells in healthy individuals. In mice there are about ˜10⁶ plasma cells, and in humans there are about ˜10⁹ plasma cells. There are also antigen specific LLPCs as described in Radbruch et al. (“Competence and competition: the challenge of becoming a long-lived plasma cell.” Nat Rev Immunol. 2006 October; 6(10):741-50; incorporated by reference in its entirety herein). Exposure to tetanus toxoid led to tetanus-specific IgG concentrations of 10-20 g/mL, which also implies a number of 0.8-1.6×10⁶ tetanus-specific LLPCs. However there is a competition for the LLPC to reside in the bone marrow or spleen survival niche. Plasma cell longevity in the bone marrow was shown to be more than 90 days of survival without turnover. It was shown in Radbruch et al., and Slifka et al, adoptive transfer of 1.5×10⁴ virus-specific plasma cells in absence of conditioning resulted in readily detectable anti-viral antibody (“Competence and competition: the challenge of becoming a long-lived plasma cell.” Nat Rev Immunol. 2006 October; 6(10):741-50; and “Humoral Immunity Due to Long-Lived Plasma Cells.” Immunity, Vol. 8, 363-372, March, 1998; incorporated by reference in their entireties herein). Thus, for clinical efficacy, the number of engrafted engineered plasma cells should be at least 1-10×10⁶ of edited long-lived plasma cells, and may be sufficient for many applications described herein. In some alternatives of the methods of treating, inhibiting or ameliorating a disease, such as cancer, in a subject in need or expressing a protein in a subject in need, the subject is administered at least 1-10×10⁶ of edited long-lived plasma cells that express a protein, or a composition which is used to deliver at least 1-10×10⁶ of edited long-lived plasma cells that express a protein. In some alternatives, the subject receives 1×10⁶, 2×10⁶, 3×10⁶, 4×10⁶, 5×10⁶, 6×10⁶, 7×10⁶, 8×10⁶, 9×10⁶, 10×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹ or 9×10⁹ edited long-lived plasma cells that express a protein or any number of cells in between a range defined by any two aforementioned values. In some alternatives herein, the genome editing is performed by nonpathogenic AAV mediated editing by direct homolougous recombination.

The generation of gene-edited long-lived plasma cells also requires specific steps in the editing of the primary B cells as well as optimization of B cell differentiation. To date, there is existing literature in murine as well as human B cell editing. In murine studies, sgRNA was delivered into CAS9 transgenic murine B cells which led to gene disruption of candidate genetic loci including application to sgRNA screens. It was also shown that retroviral delivery of sgRNA (plus antibiotic drug selection) in primary B cells leads to up 80% gene disruption. However, was HR not tested in the B cells, furthermore, it is not clinically feasible to used transgenic CAS9 and integrating viral vector for sgRNA delivery into B cells (“Efficient CRISPR-mediated mutagenesis in primary immune cells using CrispRGold and a C57BL/6 Cas9 transgenic mouse line.” Proc Natl Acad Sci USA. 2016 Nov. 1; 113(44):12514-12519; incorporated by reference in its entirety herein). Retroviral and LV-based, CRISPR/CAS9 delivery in murine B cells and hybridomas was also performed by Cheong et al. (“Editing of mouse and human immunoglobulin genes by CRISPR-Cas9 system.” Nat Commun. 2016 Mar. 9; 7:10934; incorporated by reference in its entirety herein). Editing was performed of the Ig locus to mediate Ig class-switch. However, the technique had very low efficiency and again, HR was not tested by Cheong et al. Additionally, the techniques provided were not clinically feasible for use in B cells because of the long-term CAS9 expression from an integrated viral vector (Cheong et al.).

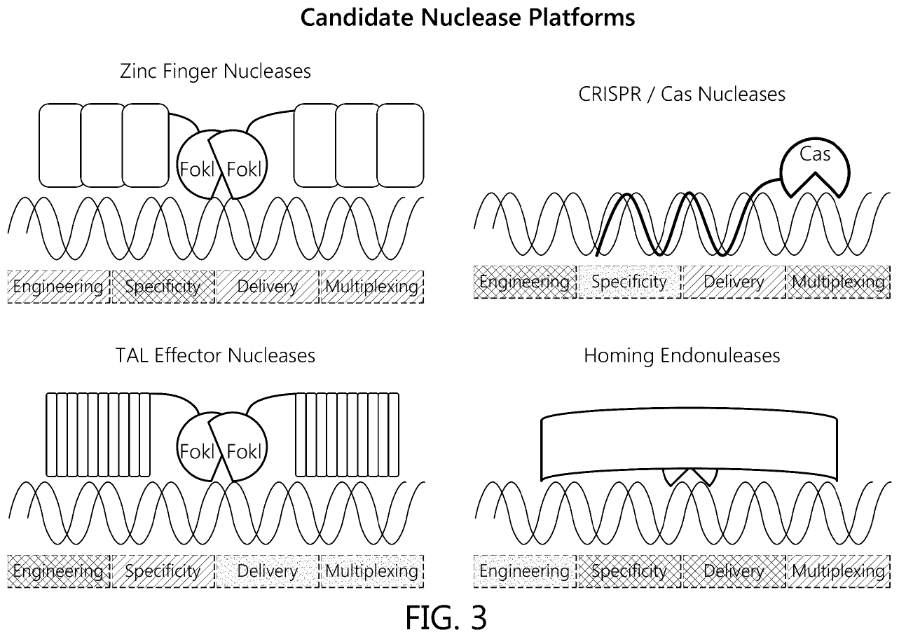

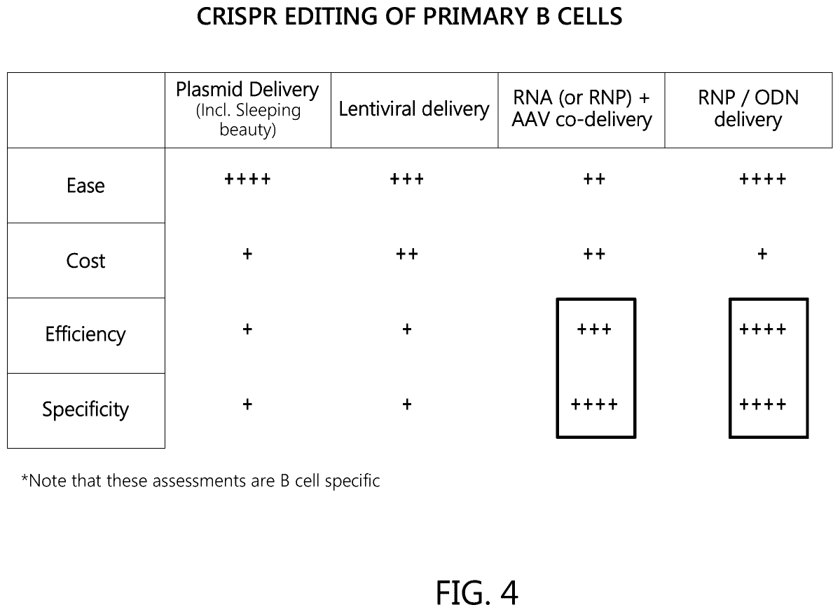

For the preferred alternatives described herein, the candidate nuclease platform used for the methods of making the plasma cell from the B cell includes genetically engineered nucleases, zinc finger nucleases, CRISPR/Cas Nucleases, TAL Effector Nucleases, and Homing Endonucleases (FIG. 3). Options for genetic modification of B cells in the alternatives described herein are RNA (or RNP) plus AAV co-delivery as well as RNP/ODN (oligodeoxynucleotides) delivery. As shown in the tables of FIG. 4, this table shows qualitative comparisons of the approaches described herein, where 4 plus indicates a positive result and the 1 plus sign describes negative outcome. These tables show that RNP+AAV or RNP+ODN are both specific, cost-effective and efficient.

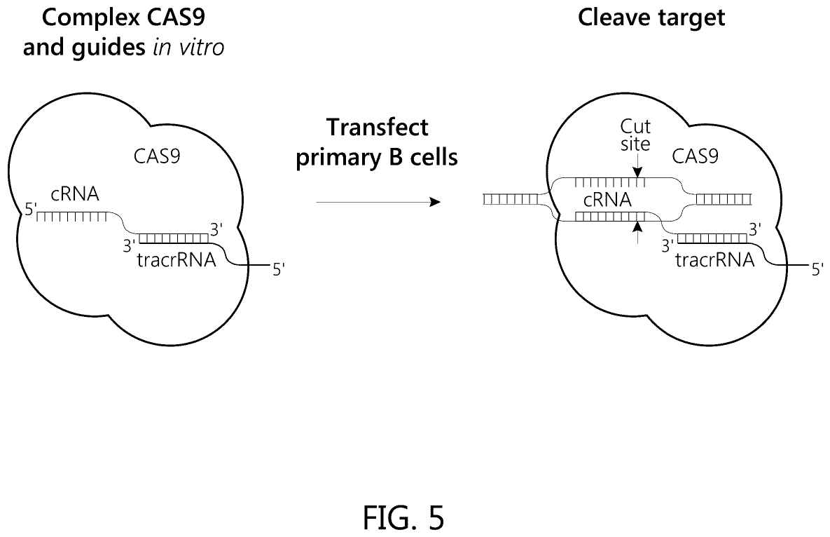

Also contemplated are ribonucleoprotein strategies for editing B cells. As shown in FIG. 5, complex CAS nuclease and RNA guides are prepared in vitro. The CAS nuclease and guides are then used to transfect the isolated primary B cells to cleave the desired target in the B cell genome. In some alternatives, the guide sequence comprises a sequence set forth in any one of SEQ ID NO: 63-112.

### Genome Modification Rates, NHEJ and Site-Specific Homology Directed Repair (HDR) Genome Editing in Primary Human B Cells.

Genome modification rates of human B cell lines as described in the alternatives herein, are 10-fold greater than previous methods for genome editing reported; and provide the first demonstration of NHEJ and, most importantly, of site-specific homology directed repair (HDR) genome editing in primary human B cells. In fact, the literature show methods that do not provide a clinically translatable methodology for use in primary human B cells. In one study, CAS9 transgenic mice were generated and utilized. In this setting, CAS9 expression was achieved using a ubiquitous transgenic expression vector and guide RNAs were subsequently introduced via lentiviral vectors into CAS9 transgenic murine B cells, as described by Chu et al. (“Efficient CRISPR-mediated mutagenesis in primary immune cells using CrispRGold and a C57BL/6 Cas9 transgenic mouse line,” Proc. Natl. Sci., Nov. 1, 2016; 113(44); 12514-12519; incorporated by reference in its entirety herein). Chu et al. did not show evidence of homology directed repair (HDR) and did not perform any work using primary human B cells. In a second report, retroviral delivery of CAS9 and guide RNAs to initiate gene disruption (NHEJ) in murine primary B cells and in human B cell lines was used by Cheong et al. (“Editing of mouse and human immunoglobulin genes by CRISPR-Cas9 system,” Nature Commun, Mar. 9, 2016; 7:10934; incorporated by reference in its entirety herein). This disruption strategy was used to initiate class switch recombination and drive switching to specific antibody constant regions. In this case, disruption percentage (NHEJ) rates were low (5-10%) and there was no demonstration of HDR. In addition, no work was performed in primary human B cells. More recently, homology directed repair (HDR) was used by Pogson et al. in B cell lines to alter the antibody specificity of a hybridoma cell line (“Immunogenomic engineering of a plug-and-(dis)-play hybridoma platform; Nat. Commun. 2016 Aug. 17; 7: 12535; incorporated by reference in its entirety herein). In this case, the repair rates were modest (5%) and the authors utilized plasmid-based DNA delivery of the repair template and the CRISPR nuclease reagents. Again, no work was performed using primary human B cells. As described herein, none of these delivery systems are likely to result in the high efficiency genome editing that have been observed in the primary B cells (rates of 80% NHEJ or rates of ˜40% HDR) of the alternatives herein. In fact, the editing rates of the alternatives described herein are surprising, because they significantly outpace the efforts of others in the literature regarding sustained gene expression following either transgene integration with lentiviral vectors or any existing genome editing approach utilized in B cells.

In some alternatives herein, the genome editing is performed by nonpathogenic AAV mediated editing by direct homolougous recombination.

The Yield of DNA Delivery is Greater than the Published Rates of DNA Delivery to Primary Human B Cells and has a Sustained Gene Expression.

The alternatives as described herein, have increased the yield of DNA delivery >1 log over published rates to primary human B cells. This advance is achieved in greatly facilitating HDR rates by either transfecting with single-stranded DNA oligonucleotides or transduction with capsid-specific AAV. Previous studies by Kim et al. using recombinant adenovirus have shown that vectors utilizing the serotype Ad-K35 can transduce primary human B cells at a rate of ˜75% (“Enhanced antitumor immunotherapeutic effect of B-cell-based vaccine transduced with modified adenoviral vector containing type 35 fiber structures.” Gene Ther. 2014, January; 21(1): 106-114). This vector serotype was designed for delivery of B cell vaccines, but in contrast to the sustained gene expression that is observed in the alternatives described herein, the HDR gene-edited loci in primary B cells, adenovirus delivery does not lead to long-term gene expression in B cells. In addition, adenoviral infected B cells are likely to be subject to immune responses in vivo that would make delivery of ex vivo adenoviral infected B cells highly problematic. In previous studies, recombinant Epstein-Barr virus (EBV) vector systems have also been developed for episomal-based gene delivery. Modified EBV vectors are capable of transducing of >80% of primary human B cells or CLL tumor cells as shown in Hellebrand et al. (“Epstein-Barr virus vector-mediated gene transfer into human B cells: potential for antitumor vaccination.” Gene Ther. 2006, January; 13(2): 150-162). However, this vector class is associated with loss in episomal DNA over time leading to absence of long-term expression; further, recombinant EBV infected B cells express EBV viral proteins and therefore remain capable of triggering T cell mediated immune responses in EBV exposed individuals (the vast majority of the human subjects). In contrast to the relative efficiency of transient gene delivery using adenovirus or EBV, integrating gamma-retroviral or lentiviral (LV) vectors are extremely inefficient for transducing human B cells as shown by Serafini et al., Bovia et al. and Janssens et al. (“Molecular evidence of inefficient transduction of proliferating human B lymphocytes by VSV-pseudotyped HIV-1-derived lentivectors.” Virology. 2004 Au 1; 325(2); 413-424, “Efficient transduction of primary human B lymphocytes and nondividing myeloma B cells with HIV-1-derived lentiviral vectors.” Blood. 2003 Mar. 1; 101(5): 1727-1733, “Efficient and stable transduction of resting B lymphocytes and primary chronic lymphocyte leukemia cells using measles virus gp displaying lentiviral vectors.” Blood. 2009 Oct. 8; 114(15): 3173-3180, “Efficiency of onco-retroviral and lentiviral gene transfer into primary mouse and human B-lymphocytes is pseudotype dependent.” Hum Gene Ther. 2003 Feb. 10; 14(3):263-76; all incorporated by reference in their entireties herein). Several studies by Amirache et al. have shown that the VSV-G envelope typically used for LV vectors is ineffective in B cells (<5% transduction) because primary B cells lack the LDL receptor used by the VSV-G coat protein (“Mystery solved: VSV-G-LVs do not allow efficient gene transfer into unstimulated T cells, B cells, and HSCs because they lack the LDL receptor.” Blood. 2014 Feb. 27; 123(9):1422-4; incorporated by reference in its entirety herein). In addition to lacking LV receptors, B cells likely express uncharacterized factors downstream of the receptor that limit LV transduction as described by Serafini et al. (“Molecular evidence of inefficient transduction of proliferating human B lymphocytes by VSV-pseudotyped HIV-1-derived lentivectors.” Virology. 2004 Aug. 1; 325(2):413-24; incorporated by reference in its entirety herein). As reported by Bovia et al., higher levels of transduction with VSV-G LV in primary human B cells (˜25%) in the setting of sustained 4-5 day co-cultures with a mouse T cell tumor line (EL-4) or following co-infection with Epstein Barr virus. However, transduction using a clinically translatable system (e.g., without tumor cell lines and using only cytokine and CD40L-based stimulation) remained very low at 2% as shown by Bovia et al. (“Efficient transduction of primary human B lymphocytes and nondividing myeloma B cells with HIV-1-derived lentiviral vectors.” Blood. 2003 Mar. 1; 101(5):1727-33, incorporated by reference in its entirety herein). Newer LV vectors employing alternative envelopes including either baboon retrovirus envelope protein (BaEV), measles virus envelope (MV) or gibbon-ape leukemia virus envelope (GALV) have demonstrated increased transduction efficiency of up to 50% with baboon endogenous retrovirus (BaEV), measles virus (MV) and Gibbon ape leukemia virus (GALV) as shown by Levy et al., (“Baboon envelope pseudotyped lentiviral vectors efficiently transduce human B cells and allow active factor IX B cell secretion in vivo in NOD/SCIDγc−/− mice.” J Thromb Haemost. 2016 December; 14(12):2478-2492; “Baboon envelope pseudotyped lentiviral vectors efficiently transduce human B cells and allow active factor IX B cell secretion in vivo in NOD/SCIDγc−/− mice.” J Thromb Haemost. 2016 December; 14(12):2478-2492; “Lentiviral vectors displaying modified measles virus gp overcome pre-existing immunity in in vivo-like transduction of human T and B cells,” Mol Ther. 2012 September; 20(9):1699-712; “Efficient transduction of healthy and malignant plasma cells by lentiviral vectors pseudotyped with measles virus glycoproteins.” Leukemia. 2012 July; 26(7):1663-70; “Measles virus glycoprotein-pseudotyped lentiviral vector-mediated gene transfer into quiescent lymphocytes requires binding to both SLAM and CD46 entry receptors.” J Virol. 2011 June; 85(12):5975-85; “Efficient and stable transduction of resting B lymphocytes and primary chronic lymphocyte leukemia cells using measles virus gp displaying lentiviral vectors.” Blood. 2009 Oct. 8; 114(15):3173-80; and “Efficient lentiviral transduction and transgene expression in primary human B cells.” Hum Gene Ther Methods. 2012 December; 23(6):408-15; all references incorporated in their entireties herein), but at the cost of drastically lower viral titers. Because the titers of BaEV, GALV and MV LV vectors are much lower (>10-20-fold lower for BaEV, GALV and 100-fold lower for MV) than LV vectors using VSV-G coats, the practicality of transducing large numbers of primary B cells is limited, because high efficiency integration requires viral quantities that result in unacceptable toxicity. In summary, the demonstration of 40% stable expression of reporter genes following HDR into target loci in primary B cells with concomitant high-levels of cell viability greatly exceeds the frequency of sustained gene expression reported for either integrating or non-integrating viral vectors, which is shown by the results of the alternative methods described herein.

Several Steps are Used to Limit the Deleterious Impact of Innate Immune Signaling that is Trigged by Foreign DNA in Primary B Cells.

As described herein, there are several steps to limit the deleterious impact of innate immune signaling triggered by foreign DNA in primary B cells. In essentially all cell types, cytosolic self- or foreign-DNA (viral, bacterial, plasmid or oligonucleotide) binds to and activates the DNA sensor cGAS, leading to synthesis of the second messenger 2′3′-cGAMP which, in turn, triggers STING-dependent downstream signaling leading to type-I interferon production and additional transcriptional changes which is described in Crowl et al. (“Intracellular Nucleic Acid Detection in Autoimmunity.” Annu Rev Immunol. 2017 Jan. 30; “cGAS-cGAMP-STING: The three musketeers of cytosolic DNA sensing and signaling.” IUBMB Life. 2016 November; 68(11):858-870). In addition to cytosolic DNA sensing, many cell types (including human B cells) express endosomal toll-like receptors (TLRs) that recognize and respond to endosomal-delivered double-stranded DNA (dsDNA) ligands, triggering MyD88 dependent signals that promote type-I interferon and pro-inflammatory gene expression. The outcome of exogenous DNA-triggered innate signals is dependent upon the route of DNA delivery, the quantity of DNA delivered, DNA structure and cell lineage. In primary B, and T lymphocytes, transfection of dsDNA oligonucleotides promotes rapid cell death via apoptosis as described by Seiffert et al., Bell et al., Van Tendeloo et al., Cotten et al., and Ebert et al. (“Efficient nucleofection of primary human B cells and B-CLL cells induces apoptosis, which depends on the microenvironment and on the structure of transfected nucleic acids.” Leukemia. 2007 September; 21(9):1977-83); “The analysis of costimulatory receptor signaling cascades in normal T lymphocytes using in vitro gene transfer and reporter gene analysis.” Nat Med. 2001 October; 7(10):1155-8; “High-level transgene expression in primary human T lymphocytes and adult bone marrow CD34+ cells via electroporation-mediated gene delivery.” Gene Ther. 2000 August; 7(16):1431-7; “Intracellular delivery of lipopolysaccharide during DNA transfection activates a lipid A-dependent cell death response that can be prevented by polymyxin B.” Hum Gene Ther. 1997 Mar. 20; 8(5):555-61.; and “Lymphocyte apoptosis: induction by gene transfer techniques.” Gene Ther. 1997 April; 4(4):296-302; all incorporated by reference in their entireties herein). Introduction of plasmid DNA by transfection in primary T lymphocytes has been tested by multiple investigators as a means to achieve transgene expression. The effectiveness of this approach has been limited by low efficiency and high toxicity. Previous studies demonstrate expression ranging from 15-75% with progressively poorer viability (<30%) as expression increases (“The analysis of costimulatory receptor signaling cascades in normal T lymphocytes using in vitro gene transfer and reporter gene analysis.” Nat Med. 2001 October; 7(10):1155-8.; “High-level transgene expression in primary human T lymphocytes and adult bone marrow CD34+ cells via electroporation-mediated gene delivery.” Gene Ther. 2000 August; 7(16):1431-7; “Gene transfection and expression in resting and activated murine CD4 T cell subsets.” J Immunol Methods. 2003 November; 282(1-2):93-102; all incorporated by reference in their entireties herein). Low cell viability correlates with transfection triggered cell apoptosis (“Lymphocyte apoptosis: induction by gene transfer techniques.” Gene Ther. 1997 April; 4(4):296-302; incorporated by reference in its entirety herein). While higher viably is observed using plasmid “Nucleofection” (perhaps due to reduced cytosolic DNA content), expression using this approach was also shown to be low (“High-efficiency transfection of primary human and mouse T lymphocytes using RNA electroporation.” Mol Ther. 2006 January; 13(1):151-9.; incorporated by reference in its entirety herein). While limited published data exist for B cells, the results from the experiments of the alternatives described herein suggest even poorer DNA transfection efficiencies with higher toxicity in primary B cells. These observations have lead most groups to abandoning plasmid DNA transfection for gene delivery. While not directly tested, cytosolic or endosomal DNA sensing likely explains this observed toxicity and strongly imply that this approach would not work for DNA template delivery for B cell genome editing. Consistent with this idea, in primary B cells plasmid DNA transfection also triggers TLR9-dependent type-I interferon and pro-inflammatory gene expression (“Oligonucleotide delivery by nucleofection does not rescue the reduced proliferation phenotype of gene-edited cells.” Nucleic Acid Ther. 2012 December; 22(6):405-13; incorporated by reference in its entirety herein); and this plasmid driven type-I interferon production is independent of CpG motifs. The alternatives described herein, avoids the toxicity problem associated with transducing primary B cells, which have not been previously reported.

Thus, triggering of these pathways is likely highly problematic for efficient B cell editing and HDR. Taking these observations into consideration, the alternative methods described herein utilize single-stranded DNA (ssDNA) delivery to limit these deleterious responses to foreign DNA. The rationale for choosing ssDNA donor templates is that the binding constant of cGAS for ssDNA (1.5 pM) is dramatically higher than that for dsDNA (87 nM), leading to the hypothesis that ssDNA is likely to elicit lower innate immune signaling in response to transfection than dsDNA (“Structure of human cGAS reveals a conserved family of second-messenger enzymes in innate immunity.” Cell Rep. 2013 May 30; 3(5):1362-8; incorporated by reference in its entirety herein). Further, ssDNA are poorly recognized by TLR9 and hence not expected to trigger TLR9 innate signaling- an additional challenge not encountered in T cell editing. Therefore two exemplary alternatives for delivery ssDNA are described: a) modified ssDNA donor oligonucleotides via transfection (“Chemically modified guide RNAs enhance CRISPR-Cas genome editing in human primary cells.” Nat Biotechnol. 2015 September; 33(9):985-9; and “Enhancing homology-directed genome editing by catalytically active and inactive CRISPR-Cas9 using asymmetric donor DNA.” Nat Biotechnol. 2016 March; 34(3):339-44; both references incorporated by reference in their entireties herein); or b) ssDNA as recombinant AAV genomes.

B Cells have a High Capacity for Site-Specific HDR Editing

The alternatives described herein have unexpectedly demonstrated that B cells have a high capacity for site-specific HDR editing. Site-specific genome editing (HDR) in ˜100% of primary B cells have been observed in which one can detect transduction or transfection with templates for homology-directed-repair by using the alternative methods of editing as described herein. While others have described methods for high rates of HDR-genome editing in primary T cells, even in the most optimized settings, HDR rates (˜20-50%) remain below the levels of AAV or oligonucleotide gene delivery (80-100%)(“Highly efficient homology-driven genome editing in human T cells by combining zinc-finger nuclease mRNA and AAV6 donor delivery.” Nucleic Acids Res. 2016 Feb. 18; 44(3):e30; Sather et al., Sci Transl Med. 2015 Sep. 30; 7(307); and “Generation of knock-in primary human T cells using Cas9 ribonucleoproteins.” Proc Natl Acad Sci USA. 2015 Aug. 18; 112(33):10437-42; both references incorporated by reference in their entireties herein). Importantly, despite advances in modulating AAV turnover using co-delivery of other viral proteins leading to marked increase in AAV genomes and/or reduced AAV turnover in T cells, HDR rates are only modestly increased (“High Efficiency CRISPR/Cas9-mediated Gene Editing in Primary Human T-cells Using Mutant Adenoviral E4orf6/Elb55k “Helper” Proteins.” Mol Ther. 2016 Sep. 29; 24(9):1570-80; incorporated by reference in its entirety herein). Thus, relative to other primary cell types commonly used for site-specific genome editing applications, a remarkable efficiency for HDR in primary human B cells have uncovered, which has not been demonstrated or previously reported. It is hypothesized that these unexpected findings showing surprisingly efficient HDR in human B cells reflects an unusual aspect of primary B cell biology and is perhaps related to an increased permissiveness to DNA breaks and resolution by HDR that has evolved to facilitate safe genomic modification during germinal center B cell activation and class-switch recombination.

### Optimal B Cell Culturing Conditions to Enable High Rates of Site-Specific Genome Editing and Subsequence Expansion and Differentiation of Gene-Edited Cells.

As described herein, the alternative methods have determined optimal B cell culturing conditions to enable high rates of site-specific genome editing and subsequent expansion and differentiation of gene edited cells. In several other primary cell types, several researchers have found that persistence in the G2 phase of cell cycle or high degrees of cell cycling promotes HDR during site-specific genome editing (“High Efficiency CRISPR/Cas9-mediated Gene Editing in Primary Human T-cells Using Mutant Adenoviral E4orf6/E1b55k “Helper” Proteins.” Mol Ther. 2016 Sep. 29; 24(9):1570-80; incorporated by reference in its entirety herein). In this exemplary alternative, the cell culture conditions were optimized to enable rapid cycling of undifferentiated B cells, and thus increased the time the cells spend in G2 (“Regulation of homologous recombination in eukaryotes.” Annu Rev Genet. 2010; 44:113-39. “Enhanced homology-directed human genome engineering by controlled timing of CRISPR/Cas9 delivery.” Elife. 2014 Dec. 15; 3:e04766; “High Efficiency CRISPR/Cas9-mediated Gene Editing in Primary Human T-cells Using Mutant Adenoviral E4orf6/Elb55k “Helper” Proteins.” Mol Ther. 2016 Sep. 29; 24(9):1570-80; incorporated by reference in its entirety herein). In some alternatives, reagents are delivered for site-specific genome editing during this cycling phase, leading to efficient HDR-gene editing. In some alternatives, the culture conditions were futher optimized, wherein the edited cell populations are expanded to high numbers and to subsequently enable the terminal differentiation of these edited and expanded B cells into long-lived plasma B cells during secondary and tertiary culture phases, respectively. This experimental design has not been previously considered for genome engineering of primary B human cells. In some alternatives herein, the gene editing is performed by nonpathogenic AAV mediated editing by direct homolougous recombination.

Several alternatives are described herein, included among these are: (a) designing editing strategies for target loci that were found in several alternatives to be highly expressed in antibody secreting B cells (JCHAIN, IGKC, IGMC, PON3, PRG2, FKBP11, SDC1, SLPI, DERL3, EDEM1, LY6C2, CRELD2, REXO2, PDIA4, PRDM1, CARDi11, CCR5 or SDF2L1, and others) and that are not subject to aberrant somatic hypermutation or required for differentiation into antibody secreting cells; (b) inserting genetic modifications that artificially induce triggerable, non-transformative expansion of edited cells including, but not limited to, disruption of HPRT locus and selection of gene edited cells using 6-TG; (c) development of a targeted sequencing method to ensure the integrity of the B cell product at loci that are known to confer susceptibility to non-Hodgkin's lymphoma or multiple myeloma; (d) designing protein producing products for clinical situations where stable delivery of the protein by a B cell provides clinical benefit by avoiding organ toxicity or immune responses observed in some AAV delivery studies (for example, Factor IX), by enabling delivery of multiple different protein simultaneously (delivery of multiple neutralizing antibodies for HIV or other pathogens), or promoting stable, steady-state levels of protein delivery over time. These would not be subject to aberrant somatic hypermutation, as previously described (“Two levels of protection for the B cell genome during somatic hypermutation.” Nature. 2008 Feb. 14; 451(7180):841-5; “DNA damage defines sites of recurrent chromosomal translocations in B lymphocytes.” Nature. 2012 Feb. 7; 484(7392):69-74; “Translocation-capture sequencing reveals the extent and nature of chromosomal rearrangements in B lymphocytes.” Cell. 2011 Sep. 30; 147(1):95-106.; all incorporated by reference in their entireties herein).

In some alternatives herein, the gene editing is performed by nonpathogenic AAV mediated editing by direct homolougous recombination.

### Development of In Vivo Pre-Clinical Models

Additional alternatives contemplated herein are in vivo pre-clinical models that can be used to assess the efficacy of the B cells. Murine models exist that could be used for this purpose.

CRISPR/Cas9, crRNA, tracrRNA

In the alternatives herein, CRISPR guide targets at the BLIMP1, IRF4, BCL6, PAX5, MITF, BACH2, IRF8 loci were designed for several alternative methods described herein, using the online MIT CRISPR design tool and the Broad Institute sgRNA design tool. A tracrRNA recognition sequence (GUUUUAGAGCUAUGCU; SEQ ID NO: 1) was added to the 3′ end of each selected guide target to form the complete synthetic crRNA sequence. The crRNA guides were synthesized by IDT® with additional modifications—phosphorothioate linkages between the four nucleotides on the 5′ end and the 3′ end, as well as 2′O-methyl groups on the three nucleotides on the 5′ end and the 3′ end). The tracrRNA with proprietary chemical modifications, as well as the recombinant Cas9 nuclease were also purchased from IDT®.

Prior to delivery of the nuclease into cells (method shown in FIG. 5), the crRNA and tracrRNA were mixed at a 1:1 molar ratio. The mixture was heat-shocked at 95° C. for 5 minutes and then incubated at room temperature for 30-60 minutes to allow crRNA: tracrRNA hybrids to form. Next, the crRNA:tracrRNA hybrids were mixed with the Cas9 nuclease at a 1.2:1 molar ratio and incubated at room temperature for 10-20 minutes to allow Cas9:crRNA:tracrRNA complexes to form. The Cas9 ribonucleoprotein (RNP) complexes were then delivered into cells by electroporation.

### Single-Stranded ODNs

All single-stranded ODNs (ssODNs) were commercially synthesized by IDT® (Ultramer® DNA Oligonucleotides) with phosphorothioate linkages between the three nucleotides on the 5′ end as well as the 3′ end.

### Production of Recombinant AAV

AAV stocks were produced as previously described in Khan et al. (Khan I F, Hirata R K, Russell D W. AAV-mediated gene targeting methods for human cells. Nature Protocols (2011) 6, 482-501.; incorporated by reference in its entirety herein). The AAV vector, serotype 6 helper plasmid (pRepCap6) and HgT1-adeno helper plasmid were transfected into HEK293T cells. Cells were harvested 48 hours later, lysed by 3 freeze-thaw cycles, treated with benzonase, and the cell lysate was purified using an iodixanol density column.

### Primary Human CD19+ B Cell Genome Editing

Peripheral blood mononuclear cells (PBMCs) collected from CD34+ negative selection flow-through from whole blood of healthy male donors post hematopoietic stem cell mobilization were purchased from Fred Hutchinson Cancer Research Center. Alternatively, PBMC were collected from healthy donors using an IRB approved protocol. CD19⁺ B cells were isolated from PBMCs by negative selection using a human B cell isolation kit (Miltenyi Biotec®, Auburn, CA) and cultured in Iscove's modified Dulbecco's medium (IMDM, Thermo Fisher Scientific®) supplemented with 10% fetal bovine serum and 55 μM beta-mercaptoethanol at 1-1.5×10⁶ cells/ml. B cells were activated with 100 ng/ml of recombinant human MEGACD40L® (Enzo Life Sciences®), 1 μg/ml of CpG oligodeoxynucleotide 2006 (Invitrogen®), 50 ng/ml of IL-2 (Peprotech®), 50 ng/ml of IL-10 (Peprotech®) and 10 ng/ml of IL-15 (Peprotech®) for 48 hours. Cells were then electroporated with Cas9 RNP complexes using the Neon Transfection System (ThermoFisher Scientific®) as follows. Cells were washed with PBS and resuspended in Neon Buffer T. 30.5pmol Cas9 RNP per 3×10⁵ cells was added to the resuspension so that the final cell density was 3×10⁷ cells/ml. Cells were then electroporated (1700V, 20 ms, 1 pulse) in 10-μl Neon tips, and then transferred into pre-warmed B cell culture medium with MEGACD40L®, CpG, IL-2, IL-10 and IL-15 and cultured at 1.5×10⁶ cells/ml. For samples transfected with an ssODN donor template, ssODN was added concurrently with Cas9 RNP at 30 pmol per 3×10⁵ cells, unless otherwise specified. For samples transduced with AAV, AAV was added to the culture immediately after electroporation. The added AAV volume was 20% of the cell culture volume, unless otherwise specified. Culture volume was doubled 24 hours after electroporation, and medium was replenished every two to three days thereafter to maintain a cell density of 1×10⁶ cells/ml.

### Flow Cytometry

Flow cytometric analysis was done on an LSR II flow cytometer (BD Biosciences®) and data were analyzed using FlowJo software (TreeStar). To assess B cell surface marker expressions, cells were stained with fluorophore-conjugated antibodies: CD19-PECy7 (clone HIB19, eBioscience®), CD27-APC (clone 0323, Life Technologies®), CD20-PE (clone L27, BD Biosciences®), CD38-PerCPCy5.5 (clone HIT2, BD Biosciences®), CD138-Alexa Fluor 700 (clone MIl5, BioLegend®), HLA-DR-FITC (clone L243, BD Biosciences®); CD19-PECy7 (clone HIB19, eBioscience®), CD27-APC (clone 0323, Life Technologies®), CD20-FITC (clone L27, BD Biosciences®), CD38-PerCPCy5.5 (clone HIT2, BD Biosciences®), CD138-Alexa Fluor 700 (clone MIl5, BioLegend®), IgD-PE (clone IA6-2, BD Biosciences®), IgM-Pacific Blue (clone MHM-88, BioLegend®). Dead cells were excluded using Fixable Live/Dead stain-Alexa Fluor 350 (LifeTechnologies®).

### Plasma Cell Differentiation Assays

In the alterantives herein, plasma cells were differentiated in vitro using a three-step culture system as previously characterized in Jourdan et al. (Jourdan M, Caraux A, De Vos J, Fiol G, Larroque M, Cognot C, Bret C, Duperray C, Hose D, Klein B. An in vitro model of differentiation of memory B cells into plasmablasts and plasma cells including detailed phenotypic and molecular characterization. Blood (2009) 114(25), 5173-5181; incorporated by reference in its entirety herein). CD19⁺ B cells isolated from PBMCs were activated for 2 days with MEGACD40L® (100 ng/ml), CpG (1 pg/ml), IL-2 (50 ng/ml), IL-10 (50 ng/ml) and IL-15 (10 ng/ml) and edited as outlined. Cells were then expanded for another 5 days in the same cocktail. Next, cells were washed and seeded in medium with IL-2 (50 ng/ml), IL-6 (50 ng/ml), IL-10 (50 ng/ml) and IL-15 (10 ng/ml) for 3 days. At day 10, cells were washed and seeded in medium with IL-6 (50 ng/ml), IL-15 (10 ng/ml) and human interferon-α 2B (100U/ml, Sigma-Aldrich®) for 3 days to stimulate plasma cell differentiation. At day 13, cell phenotypes were analyzed by immunofluorescence. To obtain a purified population of plasma cells, a BD® FACSAria II cell sorter (BD Biosciences®) was used to sort cells stained with CD27-APC (clone 0323, Life Technologies®) and CD138-Alexa Fluor 700 (clone MIl5, BioLegend®).

In the alternatives herein, experiments were performed to optimize the long-term maintenance of in vitro generated plasma cells in the culture. In order to do this, plasma cell viability were compared using the following culture systems or combinations thereof: i) plasma cell culture on M2-10B4 stromal cells as described in Cocco et al. (Cocco M, Stephenson S, Care M A, Newton D, Barnes N A, Davison A, Rawstron A, Westhead D R, Doody G M, Tooze R M. In Vitro Generation of Long-lived Human Plasma Cells. J Immunol (2012) 189(12), 5773-5785.; incorporated by reference in its entirety herein); ii) culture without stromal cells in media supplemented with combinations of IL-6 (10-50 mg/mL), APRIL (200 ng/mL), BAFF (200 ng/mL), IGF1 (10 ng/mL) and/or SDF-1 (200 ng/mL) as described in Jourdan et al. (Jourdan M, Cren M, Robert N, Bollore K, Fest T, Duperray C, Guilloton F, Hose D, Tarte K, Klein B. IL-6 supports the generation of human long-lived plasma cells in combination with either APRIL or stromal cell-soluble factors. Leukemia (2014) 28, 1647-1656.; incorporated by reference in its entirety herein). It is anticipated that these culture conditions will allow long-term maintenance of in vitro generated plasma cells for >3 months.

### Molecular Analyses—PCR, T7, Sequences

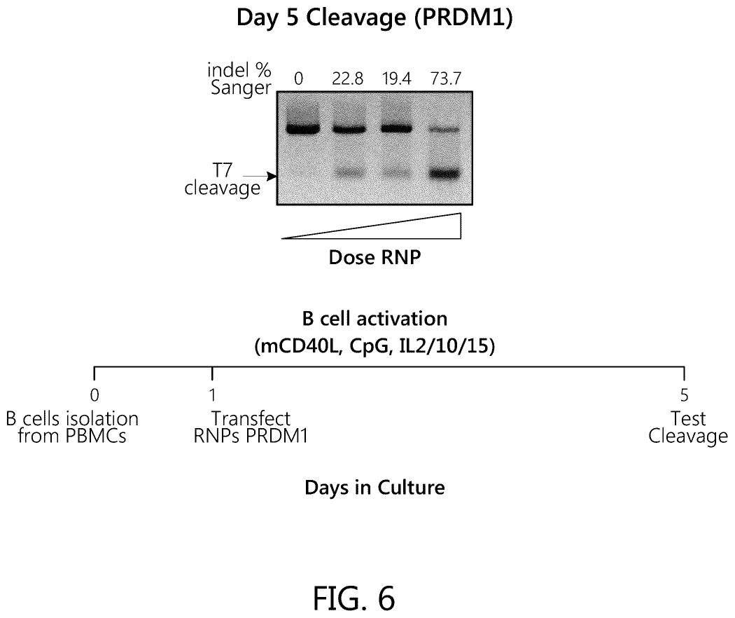

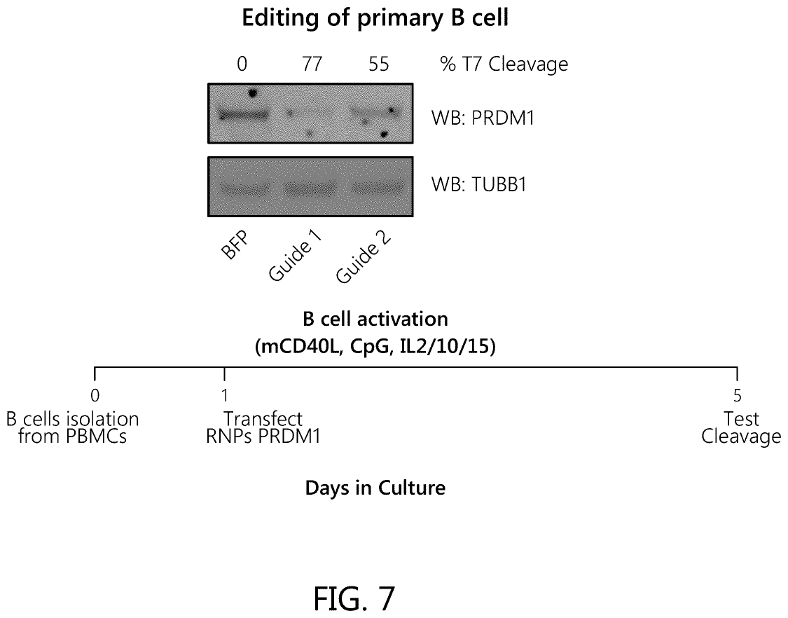

To assess the efficacy of guide RNA-CAS9 transfection for gene disruption applications in primary B cells, following transfection of PRDM1-targeting guide complexes, a variety of molecular analyses were used including a T7 endonuclease cleavage assay, Sanger sequencing and western blot. To sequence individual alleles, the DNA flanking the cleavage site was amplified via polymerase chain reaction (PCR) using locus-specific primers. Individual DNA molecules from this PCR reaction were cloned into bacterial plasmids, which were subsequently transformed to yield colonies, each expressing unique sequences. These were individually sequenced to assess the percentage of insertions or deletions initiated by the PRDM1 guides (FIG. 6). As shown in the gel, an increase in the dose of RNP led to increased cleavage. In parallel, T7 endonuclease cleavage was used of the same locus-specific PCR products as described above. To do, so the PCR products were melted by heating to 90 degrees Celsius and allowed to reanneal by lowering the temperature. Treatment of these re-annealed products with T7 endonuclease, which cleaves mis-matched DNA, initiates the formation of higher mobility DNA that enabled visualization and quantification of insertion and deletion frequency (FIG. 6). Finally, western blots using antibodies that specifically bind PRDM1 demonstrate that disruption of the gene with PRDM1 guides also blocks protein expression from that locus (FIG. 7).

### Target Guide RNA Sequences

Target guide sequences used herein are as follows in the below table:

TABLE 1
Guide RNA sequences
Methods for inducing tolerance of a peptide for
immunotherapeutic application, secreted by a B
cell in a subject in need in vivo and/or
increasing engraftment of the B cell in a
subject in need.

| Gene | Sequence | SEQ ID NO: |
|---|---|---|
| PAX5 | UGU GAA UGG ACG GCC ACU CC | 2 |
| PAX5 | UGU AGU CCG CCA GAG GAU AG | 3 |
| IRF8 | AUU GAC AGU AGC AUG UAU CC | 4 |
| IRF8 | CGG AAA UGU CCA GUU GGG AC | 5 |
| BACH2 | GUU CCU GCG CAU GCA CAA CC | 6 |
| BACH2 | CUG UGA CGU GAC UUU GAU CG | 7 |
| CCR5 | CAA UGU GUC AAC UCU UGA CA | 8 |
| CCR5 | GCU GUG UUU GCG UCU CUC CC | 9 |
| CARD11 | CAAUGACCUUACACUGACGC | 10 |
| PRDM1 | UGAUGGCGGUACUUCGGUUC | 11 |
| PRDM1 | AGGAUGCGGAUAUGACUCUG | 12 |
| PRDM1 | GGGGAGCGAGUGAUGUACGU | 13 |

In some alternatives, a method of making B cells, plasma cells or plasma cell precursors that expresses a macromolecule is provided. The B cell or plasma cell can be administered in some alternative methods described herein, with a second cell that expresses a macromolecule that promotes tolerance of a protein or a peptide and/or promotes engraftment of the B cell or plasma cell. Thus, the two cells can be administered in parallel in order to provide a subject with a therapeutic protein as well as a second protein that allows sustained protein replacement in a subject in need.

The method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the macromolecule. In some alternatives, the macromolecule is a protein, protein mimetic or peptide.

In some alternatives, the protein, protein mimetic, or peptide comprises natural amino acids and/or unnatural amino acids.

In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. Mixtures of different types of B cells can also be utilized. As described herein, B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof.

In some alternatives, the B cells in step (a) comprise memory B cells and/or naïve B cells.

In some alternatives, the developing of the B cells is performed after the B cells are arrested at a specific phase of development and/or activating the B cells such that the B cells are permissive for recombination without further B cell differentiation.

In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell.

In some alternatives, performing the first round of genome editing of the B cells for protein expression is performed in the absence of viral integration.

In some alternatives, performing the first round of genome editing of the B cells for protein expression is performed by introduction of a single stranded nucleic acid.

In some alternatives, the B cells are subject derived or are allogeneic peripheral blood mononuclear cells.

In some alternatives, the B cells are blood-derived human B cells.

In some alternatives, the first round of genome editing is performed by an RNA and protein based transfection.

In some alternatives, the first round of genome editing comprises delivering a nuclease, wherein the nuclease targets at least one genetic loci in the B cell.

In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1.

In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease.

In some alternatives, the Cas nuclease comprises Cas1, Cas2, Cas3, Cas4, Cas5, Cas6, Cas7, Cas8 or Cas9.

In some alternatives, the nuclease or synthetic guide RNAs targeting clustered regularly interspersed short palindromic repeat DNA (CRISPR) coupled to the CAS nuclease are delivered via translatable RNA or recombinant protein. In some alternatives, the guide sequence comprises a sequence set forth in any one of SEQ ID NO: 2-13 or 63-112.

In some alternatives, the first round of genome editing comprises transfecting with single stranded DNA oligonucleotides for homologous recombination into a candidate genetic loci. In some alternatives, the single stranded DNA oligonucleotides are unmodified. In other alternatives, the single stranded DNA oligonucleotides are modified with 5′ or 3′ phosphorothioate linkages as repair templates.

In some alternatives, the first round of genome editing comprises transducing the B cell with a recombinant adeno-associated virus vector to serve as a donor template for homologous recombination into a candidate genetic loci.

In some alternatives, the recombinant adeno-associated virus vector is single-stranded, double stranded or self-complementary.

In some alternatives, the method further comprises applying methods to prevent somatic hypermutation of an antibody locus in the B cell.

In some alternatives, the method to prevent somatic hypermutation of an antibody locus in the B cell comprises disruption of an AID gene.

In some alternatives, the method further comprises selectively increasing a proportion of gene edited B cells.

In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cell short lived drug activatable growth enhancers; and (d) inserting genetic modifications that artificially induce non-transformative expansion of gene edited B cells. In some alternatives, the first round of genome editing further comprises techniques for homology-directed repair.

In some alternatives, the second round of genome editing results in the excision of the IgM constant region.

In some alternatives, the second round of genome editing results in the B cells mimicking a class-switch to IgG1 positive cells.

In some alternatives, step (a) of the step increasing the proportion of gene edited B cells further comprises removing IgM positive cells.

In some alternatives, method further comprises activating the IgG1 positive cells and expanding the IgG1 positive cells, wherein the expanding is performed by exogenous antigens that specifically bind the inserted surface-expressed IgG1.

In some alternatives, the IgM positive cells are removed by negative selection.

In some alternatives, the expanding the IgG1 positive cells is performed by exogenous antigens that specifically bind the inserted surface-expressed IgG1.

In some alternatives, the drug activatable growth enhancers expressed after the third round of genome editing are rapamycin-inducible FKBP11 dimers.

In some alternatives, the activatable growth enhancers that are RNA transfected into the B cells of step (c) are rapamycin-inducible FKBP11 dimers.

In some alternatives, the inserting genetic modifications that artificially induce triggerable, non-transformative expansion of edited cells results in disruption of a HPRT locus and negative selection of non-edited cells.

In some alternatives, the negative selecting of non-edited cells is performed by 6-thioguanine.

In some alternatives, the method further comprising introducing nucleic acid, wherein the nucleic acid encodes at least one cell surface protein.

In some alternatives, the at least one cell surface protein is CD20.

In some alternatives, the first round of genome editing further comprises cycling the B-cells for homologous recombination of the single stranded DNA oligonucleotides or recombinant adeno-associated virus into the candidate genetic loci.

In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned lengths.

In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells and/or any mixtures or combinations thereof.

In some alternatives, the isolating is performed by isolation of naïve or memory B cells.

In some alternatives, the isolating is performed from isolation from healthy donor PBMCs or cell apheresis collections.

In some alternatives, the differentiating step is performed in a three-step culture system comprising an activation and proliferation step, a plasmablast differentiation step, and a plasma cell differentiation step.

In some alternatives, the activation and proliferation step is performed in the presence of any combination of MCD40L (CD40 trimer), CpG, IL-2, IL-10 and/or IL-15.

In some alternatives, the plasmablast differentiation step is performed in the presence any combination of IL-2, IL-6, IL-10 and/or IL-15.

In some alternatives, the plasma cell differentiation step is performed in the presence any combination of IL-6, IL-15, APRIL and/or IFNα.

In some alternatives, the single stranded DNA oligonucleotides or the recombinant adenovirus comprises a sequence encoding the protein.

In some alternatives, the protein comprises an enzyme, monoclonal antibody or a binding portion thereof, neutralizing antibodies or a binding portions thereof, therapeutic antibodies or binding portions thereof, cytokine, cytokine receptor, complement protein, inhibitory protein, anti-fibrotic molecule, anti-thrombotic molecule, coagulation factor, glucose response element or a synthetic engineered protein.

In some alternatives, the protein is Factor VIII, Factor IX, SERPING1 or SERPINA1, wherein the protein is Factor VIII, Factor IX, SERPING1 or SERPINA1.

In some alternatives, the enzyme is ADAMTS13, LIPA, GLA, or ALPL.

In some alternatives, the protein is a receptor antagonist for treatment of periodic fever/autoinflammatory syndromes or complement inhibitory proteins.

In some alternatives, the compliment inhibitory protein is Factor H, Factor I or a C1 inhibitor.

In some alternatives, the protein is an anti-fibrotic molecule, wherein the anti-fibrotic molecule is SCGB1A1.

In some alternatives, the therapeutic antibody or a binding portion thereof is specific for a protein or other molecule expressed in autoimmune disorders, autoinflammatory disorders, immune dysregulation and/or cancer.

In some alternatives, the antibody is an anti-IL1 monoclonal antibody, anti-TNF antibody, anti-IL33 antibody or anti-C5 antibody or a binding portion of any one or more of these antibodies.

In some alternatives, the protein is an anti-thrombotic molecule, wherein the anti-thrombotic molecule is APLN.

In some alternatives, the protein comprises an anti-PCSK9 inhibitory antibody or a binding portion thereof.

In some alternatives, the protein comprises broadly neutralizing HIV-1 antibodies (bNAbs), or a binding portion thereof.

In some alternatives, the protein is an engineered protein, wherein the engineered protein is protective in viral, fungal, parasitic or bacterial infection.

In some alternatives, the engineered protein comprises an antibody or a binding portion thereof, wherein the antibody or a binding portion thereof is specific for an antigen that is expressed by a virus, fungus, parasite or bacteria.

In some alternatives, the protein comprises an antibody or a binding portion thereof, wherein the antibody or a binding portion thereof is specific for an antigen that is expressed in a viral, fungal, parasitic or bacterial infection.

In some alternatives, the B cell or plasma cell manufactured by any one of the alternatives described herein is provided. In some alternatives, the B cell or plasma cell that expresses the macromolecule is provided to subject in need. In some alternatives, the subject has a disease. In some alternatives, the subject is in need of engraftment of the B cell or plasma cell. In some alternatives, a method of inducing tolerance of a peptide for immunotherapeutic application, secreted by a B cell in a subject in need in vivo and/or increasing engraftment or survival of the B cell in a subject in need.

In some alternatives, the method comprises administering the plasma cell or B cell manufactured by any one of the alternatives described herein, wherein the plasma cell or B cell expresses a macromolecule for therapy or a peptide for immunotherapy and administration of a second B cell. In some alternatives, the second B cell expresses a second macromolecule, wherein the second macromolecule promotes tolerance of a peptide or protein for therapeutic application and/or engraftment. In some alternatives, the second macromolecule comprises IFN-alpha, BAFF, APRIL, IL-10 or IL-6.

Gene editing can be performed to manufacture an active B cell line that also co-expresses inhibitory cytokines such as IL-10.

In some alternatives, of the methods for inducing tolerance, about 700K cells/mL for a subject in need of treatment or therapy, may be therapeutic. That equates to ˜700 million cells per liter in a person. These cells will likely dissipate within a month, but could provide therapeutic benefit for much longer periods. For inflammatory disease, these treatments would likely have to be repeated. For graft tolerization, it may be at least a one-time therapy.

### Alternative 1: Editing of Primary B Cells

As shown in FIG. 7, primary B cells were edited. Peripheral blood mononuclear cells (PBMCs) collected from CD34+ negative selection flow-through from whole blood of healthy male donors post hematopoietic stem cell mobilization were purchased from Fred Hutchinson Cancer Research Center. Alternatively, PBMC were collected from healthy donors using an IRB approved protocol. CD19+ B cells were isolated from PBMCs by negative selection using a human B cell isolation kit (Miltenyi Biotec®, Auburn, CA) and cultured in Iscove's modified Dulbecco's medium (IMDM, Thermo Fisher Scientific®) supplemented with 10% fetal bovine serum and 55 μM beta-mercaptoethanol at 1-1.5×10⁶ cells/ml. B cells were activated with 100 ng/ml of recombinant human MEGACD40L® (Enzo Life Sciences®), 1 pg/ml of CpG oligodeoxynucleotide 2006 (Invitrogen®), 50 ng/ml of IL-2 (Peprotech®), 50 ng/ml of IL-10 (Peprotech®) and 10 ng/ml of IL-15 (Peprotech®) for 48 hours. Cells were then electroporated with Cas9 RNP complexes using the Neon Transfection System (ThermoFisher Scientific®) as follows. Cells were washed with PBS and resuspended in Neon Buffer T. 30.5pmol Cas9 RNP per 3×10⁵ cells was added to the resuspension so that the final cell density was 3×10⁷ cells/ml. Cells were then electroporated (1700V, 20 ms, 1 pulse) in 10-μl Neon tips, and then transferred into pre-warmed B cell culture medium with MEGACD40L®, CpG, IL-2, IL-10 and IL-15 and cultured at 1.5×10⁶ cells/ml. For samples transfected with an ssODN donor template, ssODN was added concurrently with Cas9 RNP at 30 pmol per 3×10⁵ cells, unless otherwise specified. For samples transduced with AAV, AAV was added to the culture immediately after electroporation. The added AAV volume was 20% of the cell culture volume, unless otherwise specified. Culture volume was doubled 24 hours after electroporation, and medium was replenished every two to three days thereafter to maintain a cell density of 1×10⁶ cells/ml.

### Alternative 2: PRDM1 Disruption Limits Plasmablast Generation

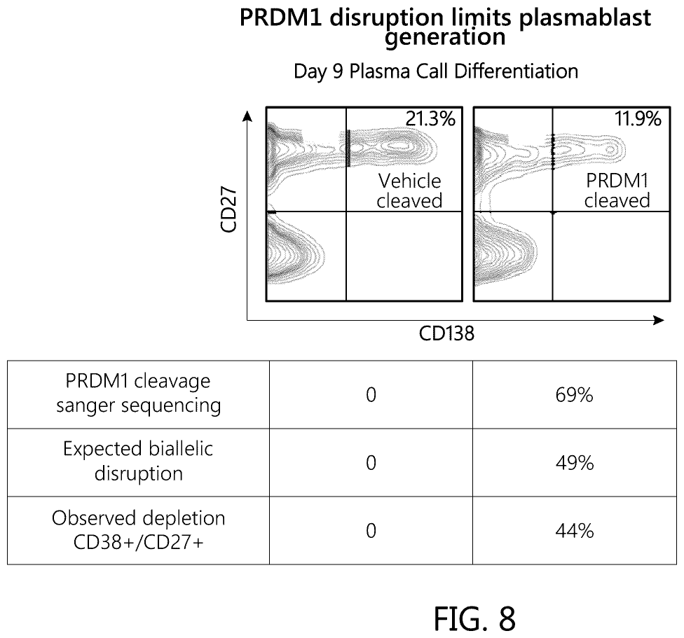

Plasma cell differentiation was performed as described in the section entitled “Plasma cell differentiation assays” of this paper. Sanger sequencing was used to calculate the total allelic disruption frequencies of PRDM1 in this experiment (69%). Based on these numbers, the expected biallelic disruption frequency was 49%. Because biallelic disruption or PRDM1 blocks plasmablast differentiation, it is expected that the number of plasmablasts to decrease ˜49%. To quantify plasmablasts, flow cytometry was used. Flow cytometric analysis was done on an LSR II flow cytometer (BD Biosciences®) and data were analyzed using FlowJo software (TreeStar). To assess B cell surface marker expressions, cells were stained with fluorophore-conjugated antibodies: CD19-PECy7 (clone HIB19, eBioscience®), CD27-APC (clone 0323, Life Technologies®), CD20-PE (clone L27, BD Biosciences®), CD38-PerCPCy5.5 (clone HIT2, BD Biosciences®), CD138-Alexa Fluor 700 (clone MIl5, BioLegend®), HLA-DR-FITC (clone L243, BD Biosciences®); CD19-PECy7 (clone HIB19, eBioscience®), CD27-APC (clone 0323, Life Technologies®), CD20-FITC (clone L27, BD Biosciences®), CD38-PerCPCy5.5 (clone HIT2, BD Biosciences®), CD138-Alexa Fluor 700 (clone MIl5, BioLegend®), IgD-PE (clone IA6-2, BD Biosciences®), IgM-Pacific Blue (clone MHM-88, BioLegend®). Dead cells were excluded using Fixable Live/Dead stain-Alexa Fluor 350 (LifeTechnologies®). Consistent with the hypothesis, the observed depletion of plasmablasts (CD38+/CD27+) was at 44%, which closely mimics the observed bi-allelic disruption frequency (FIG. 8).

### Alternative 3: Homologous-Directed Repair (HDR) Using Single-Stranded DNA Templates

Homologous directed repair was performed using single stranded ODNs. The ssODNs were commercially synthesized by IDT® (Ultramer® DNA Oligonucleotides) with phosphorothioate linkages between the three nucleotides on the 5′ end as well as the 3′ end. For samples transfected with an ssODN donor template, ssODN was added concurrently with Cas9 RNP at 30 pmol per 3×10⁵ cells, unless otherwise specified.

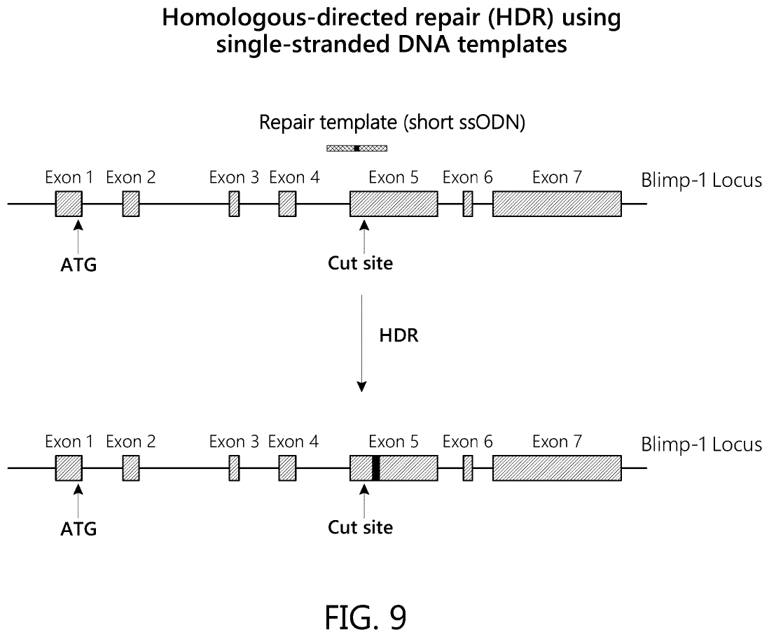

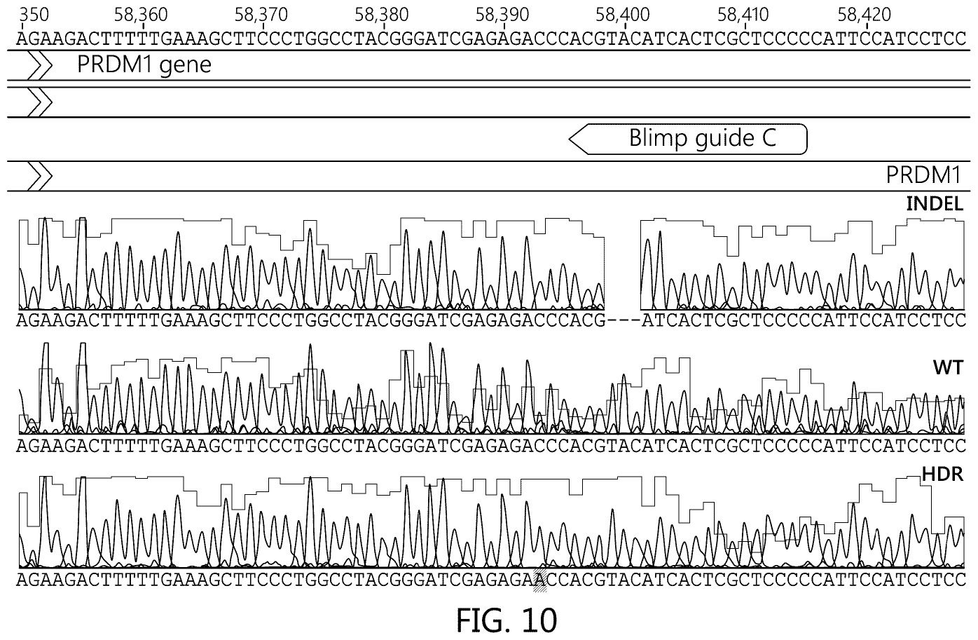

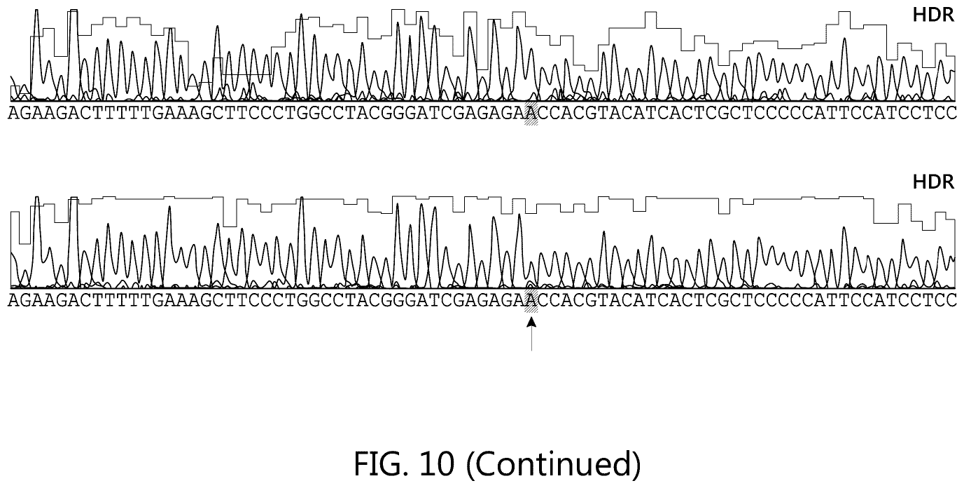

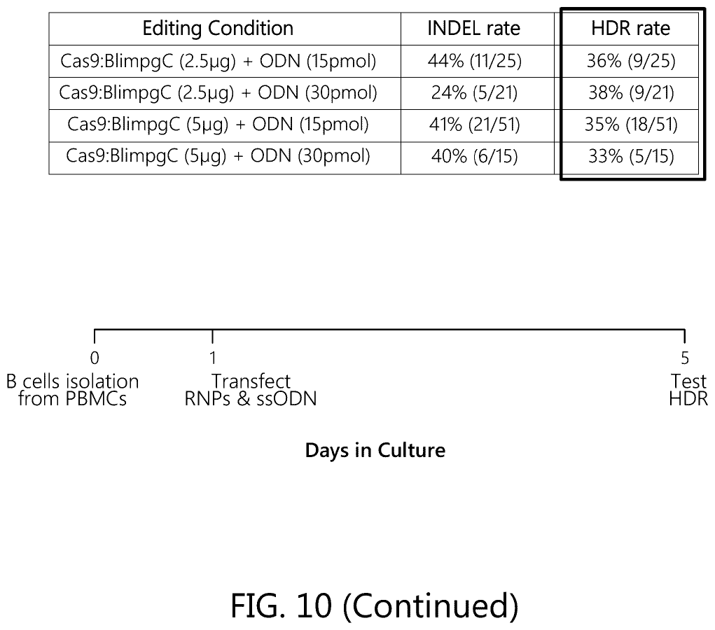

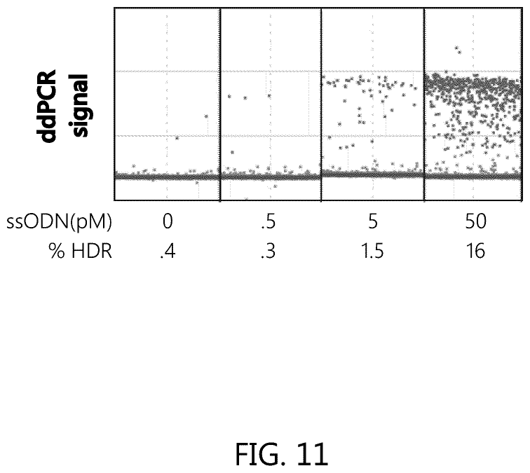

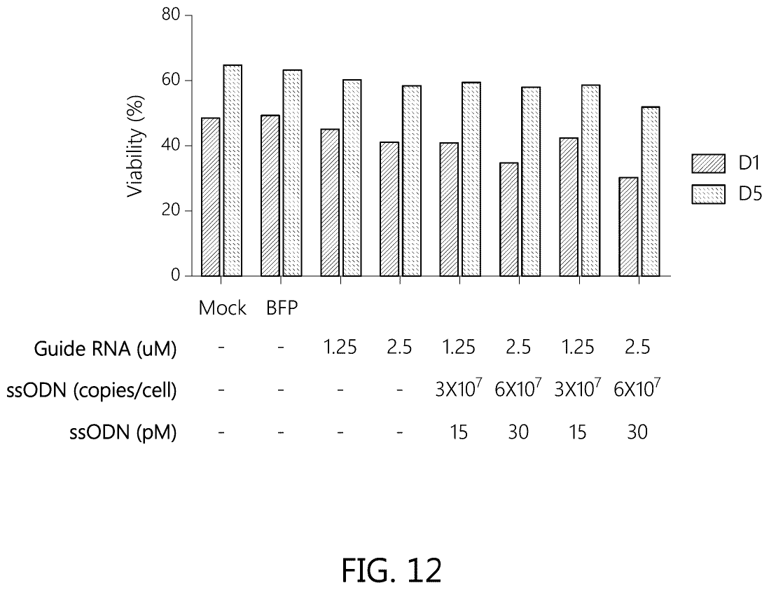

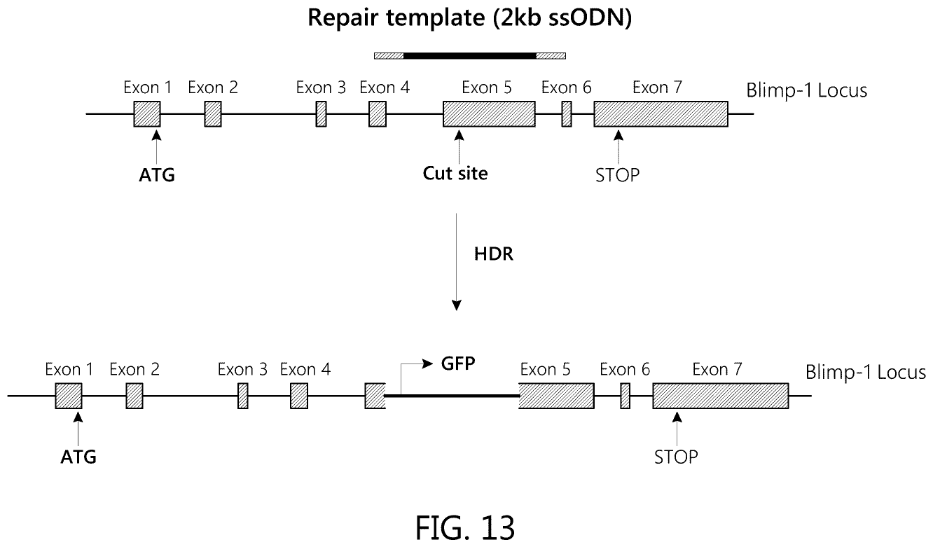

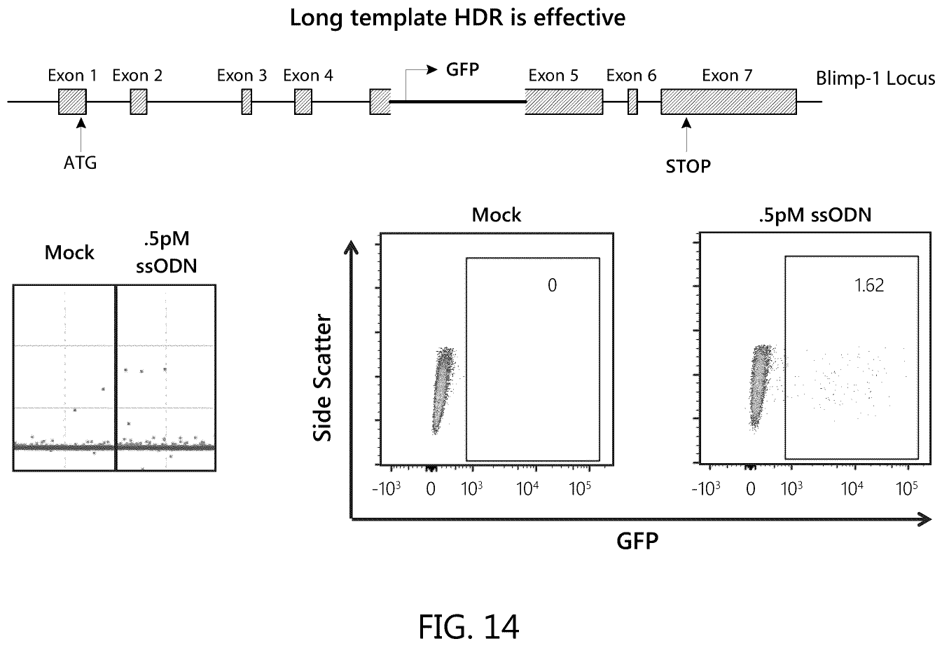

The expression of the Blimp-1 protein (PR domain zinc finger protein 1) in B lymphocytes can lead to an immune response through proliferation and differentiation of antibody secreting plasma cells. As schematized in FIG. 9, the initial strategy for testing HDR in B cells was to use single-stranded oligonucleotides (ssODNs) to insert sequence into the PRDM1 locus at Exon 5 (FIG. 9-14). This site is shown in the light grey in Exon 5 of FIG. 9. Varying amounts of Cas9:PRDM1-C with the single stranded ODNs were tested to see how they both can affect HDR rate. These were initially analyzed by PCR cloning combined with colony sequencing (Please see section entitled Molecular Analyses-PCT, T7, sequences). The sequencing traces showed the single base-change which were used to assess HDR, which was quantified (FIG. 10, bold dark box under the table headed HDR rate). Next, allele-specific digital PCR was used to quantify HDR rates over a wider range of repair template concentration and found that 50 pM ssODN resulted in the highest rates of HDR (FIG. 11). In the same experiment, B cell viability was quantified by flow cytometry (See section entitled PRDM1 disruption limits plasmablast generation for methods of flow cytometry), demonstrating that although 30 pM ssODN combined with 2.5 μM guide RNA results in a decrease of viability at day 1, the cells recover by day 5 (FIG. 12). As shown in FIG. 12, the days are consecutively D1 followed by D5 on the bar graphs. Next, the ssODN approach was expanded to include larger oligonucleotides (schematized in FIG. 13). As shown in FIG. 13, the repair template is inserted within exon 5 at the cutsite. As shown in FIG. 14, long template HDR is also effective in the editing of B cells, which has not been previously reported. In summary, there was surprisingly low toxicity as well as high-efficiency HDR as compared to using high titer AAV repair templates, which were shown to cause toxicity at 3×10⁵ copies/cell.

The AAV repair template sequences are shown below as follows:

| 1079_pscAAV-MND.GFP | |
|---|---|
| (SEQ ID NO: 14) | |
| 1 | AAGCTTCCCG GGGGGATCTG GGCCACTCCC TCTCTGCGCG CTCGCTCGCT CACTGAGGCC |
| 61 | GGGCGACCAA AGGTCGCCCG ACGCCCGGGC TTTGCCCGGG CGGCCTCAGT GAGCGAGCGA |
| 121 | GCGCGCAGAG AGGGAGTGGC CAACTCCATC ACTAGGGGTT CCTGGAGGGG TGGAGTCGTG |
| 181 | ACCTAGGGAA CAGAGAAACA GGAGAATATG GGCCAAACAG GATATCTGTG GTAAGCAGTT |
| 241 | CCTGCCCCGG CTCAGGGCCA AGAACAGTTG GAACAGCAGA ATATGGGCCA AACAGGATAT |
| 301 | CTGTGGTAAG CAGTTCCTGC CCCGGCTCAG GGCCAAGAAC AGATGGTCCC CAGATGCGGT |
| 361 | CCCGCCCTCA GCAGTTTCTA GAGAACCATC AGATGTTTCC AGGGTGCCCC AAGGACCTGA |
| 421 | AATGACCCTG TGCCTTATTT GAACTAACCA ATCAGTTCGC TTCTCGCTTC TGTTCGCGCG |
| 481 | CTTCTGCTCC CCGAGCTCTA TATAAGCAGA GCTCGTTTAG TGAACCGTCA GATCGCCTGG |
| 541 | AGACGCCATC CACGCTGTTT TGACTTCCAT AGAAGGATCC TCGAGGCCAC CATGGTGAGC |
| 601 | AAGGGCGAGG AGCTGTTCAC CGGGGTGGTG CCCATCCTGG TCGAGCTGGA CGGCGACGTA |
| 661 | AACGGCCACA AGTTCAGCGT GTCCGGCGAG GGCGAGGGCG ATGCCACCTA CGGCAAGCTG |
| 721 | ACCCTGAAGT TCATCTGCAC CACCGGCAAG CTGCCCGTGC CCTGGCCCAC CCTCGTGACC |
| 781 | ACCCTGACCT ACGGCGTGCA GTGCTTCAGC CGCTACCCCG ACCACATGAA GCAGCACGAC |
| 841 | TTCTTCAAGT CCGCCATGCC CGAAGGCTAC GTCCAGGAGC GCACCATCTT CTTCAAGGAC |
| 901 | GACGGCAACT ACAAGACCCG CGCCGAGGTG AAGTTCGAGG GCGACACCCT GGTGAACCGC |
| 961 | ATCGAGCTGA AGGGCATCGA CTTCAAGGAG GACGGCAACA TCCTGGGGCA CAAGCTGGAG |
| 1021 | TACAACTACA ACAGCCACAA CGTCTATATC ATGGCCGACA AGCAGAAGAA CGGCATCAAG |
| 1081 | GTGAACTTCA AGATCCGCCA CAACATCGAG GACGGCAGCG TGCAGCTCGC CGACCACTAC |
| 1141 | CAGCAGAACA CCCCCATCGG CGACGGCCCC GTGCTGCTGC CCGACAACCA CTACCTGAGC |
| 1201 | ACCCAGTCCG CCCTGAGCAA AGACCCCAAC GAGAAGCGCG ATCACATGGT CCTGCTGGAG |
| 1261 | TTCGTGACCG CCGCCGGGAT CACTCTCGGC ATGGACGAGC TGTACAAGTA AGCGGCCGCA |
| 1321 | ATTCACCCCA CCAGTGCAGG CTGCCTATCA GAAAGTGGTG GCTGGTGTGG CTAATGCCCT |
| 1381 | GGCCCACAAG TATCACTAAG CTCGCTTTCT TGCTGTCCAA TTTCTATTAA AGGTTCCTTT |
| 1441 | GTTCCCTAAG TCCAACTACT AAACTGGGGG ATATTATGAA GGGCCTTGAG CATCTGGATT |
| 1501 | CTGCCTAATA AAAAACATTT ATTTTCATTG CAATGATGTA TTTAAATTAT TTCTGAATAT |
| 1561 | TTTACTAAAA AGGGAATGTG GGAGGTCAGT GCATTTAAAA CATAAAGAAA TGAAGAGCTA |
| 1621 | GTTCAAACCT TGGGAAAATA CACTATATCT TAAACTCCAT GAAAGAAGGT GAGGCTGCAA |
| 1681 | ACAGCTAATG CACATTGGCA ACAGCCCCTG ATGCCTATGC CTTATTCATC CCTCAGAAAA |
| 1741 | GGATTCAAGT AGAGGCTTGA TTTGGAGGTT AAAGTTTTGC TATGCTGTAT TTTACATTAC |
| 1801 | TTATTGTTTT AGCTGTCCTC ATGAATGTCT TTTCACTACC CATTTGCTTA TCCTGCATCT |
| 1861 | CTCAGCCTTG ACTCCACTCA GTTCTCTTGC TTAGAGATAC CACCTTTCCC CTGAAGTGTT |
| 1921 | CCTTCCATGT TTTACGGCGA GATGGTTTCT CCTCGCCTGG CCACTCAGCC TTAGTTGTCT |
| 1981 | CTGTTGTCTT ATAGAGGTCT ACTTGAAGAA GGAAAAACAG GGGGCATGGT TTGACTGTCC |
| 2041 | TGTGAGCCCT TCTTCCCTGC CTCCCCCACT CACAGTGACA CTAGTCCACT CCCTCTCTGC |
| 2101 | GCGCTCGCTC GCTCACTGAG GCCGGGCGAC CAAAGGTCGC CCGACGCCCG GGCTTTGCCC |
| 2161 | GGGCGGCCTC AGTGAGCGAG CGAGCGCGCA GAGAGGGACA GATCCGGGCC CGCATGCGTC |
| 2221 | GACAATTCAC TGGCCGTCGT TTTACAACGT CGTGACTGGG AAAACCCTGG CGTTACCCAA |
| 2281 | CTTAATCGCC TTGCAGCACA TCCCCCTTTC GCCAGCTGGC GTAATAGCGA AGAGGCCCGC |
| 2341 | ACCGATCGCC CTTCCCAACA GTTGCGCAGC CTGAATGGCG AATGGCGCCT GATGCGGTAT |
| 2401 | TTTCTCCTTA CGCATCTGTG CGGTATTTCA CACCGCATAT GGTGCACTCT CAGTACAATC |
| 2461 | TGCTCTGATG CCGCATAGTT AAGCCAGCCC CGACACCCGC CAACACCCGC TGACGCGCCC |
| 2521 | TGACGGGCTT GTCTGCTCCC GGCATCCGCT TACAGACAAG CTGTGACCGT CTCCGGGAGC |
| 2581 | TGCATGTGTC AGAGGTTTTC ACCGTCATCA CCGAAACGCG CGAGACGAAA GGGCCTCGTG |
| 2641 | ATACGCCTAT TTTTATAGGT TAATGTCATG ATAATAATGG TTTCTTAGAC GTCAGGTGGC |
| 2701 | ACTTTTCGGG GAAATGTGCG CGGAACCCCT ATTTGTTTAT TTTTCTAAAT ACATTCAAAT |
| 2761 | ATGTATCCGC TCATGAGACA ATAACCCTGA TAAATGCTTC AATAATATTG AAAAAGGAAG |
| 2821 | AGTATGAGTA TTCAACATTT CCGTGTCGCC CTTATTCCCT TTTTTGCGGC ATTTTGCCTT |
| 2881 | CCTGTTTTTG CTCACCCAGA AACGCTGGTG AAAGTAAAAG ATGCTGAAGA TCAGTTGGGT |
| 2941 | GCACGAGTGG GTTACATCGA ACTGGATCTC AACAGCGGTA AGATCCTTGA GAGTTTTCGC |
| 3001 | CCCGAAGAAC GTTTTCCAAT GATGAGCACT TTTAAAGTTC TGCTATGTGG CGCGGTATTA |
| 3061 | TCCCGTATTG ACGCCGGGCA AGAGCAACTC GGTCGCCGCA TACACTATTC TCAGAATGAC |
| 3121 | TTGGTTGAGT ACTCACCAGT CACAGAAAAG CATCTTACGG ATGGCATGAC AGTAAGAGAA |
| 3181 | TTATGCAGTG CTGCCATAAC CATGAGTGAT AACACTGCGG CCAACTTACT TCTGACAACG |
| 3241 | ATCGGAGGAC CGAAGGAGCT AACCGCTTTT TTGCACAACA TGGGGGATCA TGTAACTCGC |
| 3301 | CTTGATCGTT GGGAACCGGA GCTGAATGAA GCCATACCAA ACGACGAGCG TGACACCACG |
| 3361 | ATGCCTGTAG CAATGGCAAC AACGTTGCGC AAACTATTAA CTGGCGAACT ACTTACTCTA |
| 3421 | GCTTCCCGGC AACAATTAAT AGACTGGATG GAGGCGGATA AAGTTGCAGG ACCACTTCTG |
| 3481 | CGCTCGGCCC TTCCGGCTGG CTGGTTTATT GCTGATAAAT CTGGAGCCGG TGAGCGTGGG |
| 3541 | TCTCGCGGTA TCATTGCAGC ACTGGGGCCA GATGGTAAGC CCTCCCGTAT CGTAGTTATC |
| 3601 | TACACGACGG GGAGTCAGGC AACTATGGAT GAACGAAATA GACAGATCGC TGAGATAGGT |
| 3661 | GCCTCACTGA TTAAGCATTG GTAACTGTCA GACCAAGTTT ACTCATATAT ACTTTAGATT |
| 3721 | GATTTAAAAC TTCATTTTTA ATTTAAAAGG ATCTAGGTGA AGATCCTTTT TGATAATCTC |
| 3781 | ATGACCAAAA TCCCTTAACG TGAGTTTTCG TTCCACTGAG CGTCAGACCC CGTAGAAAAG |
| 3841 | ATCAAAGGAT CTTCTTGAGA TCCTTTTTTT CTGCGCGTAA TCTGCTGCTT GCAAACAAAA |
| 3901 | AAACCACCGC TACCAGCGGT GGTTTGTTTG CCGGATCAAG AGCTACCAAC TCTTTTTCCG |
| 3961 | AAGGTAACTG GCTTCAGCAG AGCGCAGATA CCAAATACTG TTCTTCTAGT GTAGCCGTAG |
| 4021 | TTAGGCCACC ACTTCAAGAA CTCTGTAGCA CCGCCTACAT ACCTCGCTCT GCTAATCCTG |
| 4081 | TTACCAGTGG CTGCTGCCAG TGGCGATAAG TCGTGTCTTA CCGGGTTGGA CTCAAGACGA |
| 4141 | TAGTTACCGG ATAAGGCGCA GCGGTCGGGC TGAACGGGGG GTTCGTGCAC ACAGCCCAGC |
| 4201 | TTGGAGCGAA CGACCTACAC CGAACTGAGA TACCTACAGC GTGAGCTATG AGAAAGCGCC |
| 4261 | ACGCTTCCCG AAGGGAGAAA GGCGGACAGG TATCCGGTAA GCGGCAGGGT CGGAACAGGA |
| 4321 | GAGCGCACGA GGGAGCTTCC AGGGGGAAAC GCCTGGTATC TTTATAGTCC TGTCGGGTTT |
| 4381 | CGCCACCTCT GACTTGAGCG TCGATTTTTG TGATGCTCGT CAGGGGGGCG GAGCCTATGG |
| 4441 | AAAAACGCCA GCAACGCGGC CTTTTTACGG TTCCTGGCCT TTTGCTGGCC TTTTGCTCAC |
| 4501 | ATGTTCTTTC CTGCGTTATC CCCTGATTCT GTGGATAACC GTATTACCGC CTTTGAGTGA |
| 4561 | GCTGATACCG CTCGCCGCAG CCGAACGACC GAGCGCAGCG AGTCAGTGAG CGAGGAAGCG |
| 4621 | GAAGAGCGCC CAATACGCAA ACCGCCTCTC CCCGCGCGTT GGCCGATTCA TTAATGCAGC |
| 4681 | TGGCACGACA GGTTTCCCGA CTGGAAAGCG GGCAGTGAGC GCAACGCAAT TAATGTGAGT |
| 4741 | TAGCTCACTC ATTAGGCACC CCAGGCTTTA CACTTTATGC TTCCGGCTCG TATGTTGTGT |
| 4801 | GGAATTGTGA GCGGATAACA ATTTCACACA GGAAACAGCT ATGACCATGA TTACGCCAAG |
| 4861 | CTCTCGAGAT CTAGA |
| 1347_pscAAV.Blimp.0.4 kb.MND.GFP | |
| (SEQ ID NO: 15) | |
| 1 | AAGCTTCCCG GGGGGATCTG GGCCACTCCC TCTCTGCGCG CTCGCTCGCT CACTGAGGCC |
| 61 | GGGCGACCAA AGGTCGCCCG ACGCCCGGGC TTTGCCCGGG CGGCCTCAGT GAGCGAGCGA |
| 121 | GCGCGCAGAG AGGGAGTGGC CAACTCCATC ACTAGGGGTT CCTGGAGGGG TGGAGTCGTG |
| 181 | ACCTAGGACG CGTGCCAGCT GTTACTCAGG TTTTCTCAAG AAGGAGGAGC AACTTTGGCA |
| 241 | GTTTTGCTTC AGTTCTCTCT AGCCCTCTGT GTAATCGCCC CTTTTTCTTT ATTTCAGCAC |
| 301 | AAACACAGAG CAGTCTAAAG CAACCGAGCA CTGAGAAAAA TGAACTCTGC CCAAAGAATG |
| 361 | TCCCAAAGAG AGAGTACAGC GTGAAAGAAA TCCTAAAATT GGACTCCAAC CCCTCCAAAG |
| 421 | GAAAGGACCT CTACCGTTCT AACATTTCAC CCCTCACATC AGAAAAGGAC CTCGATGACT |
| 481 | TTAGAAGACG TGGGAGCCCC GAAATGCCCT TCTACCCTCG GGTCGTTTAC CCCATCCGGG |
| 541 | CCCCTCTGCC AGAAGACTTT TTGAAAGCTT CCCTGGCCTA CGGGATCGAG AGAGAACAGA |
| 601 | GAAACAGGAG AATATGGGCC AAACAGGATA TCTGTGGTAA GCAGTTCCTG CCCCGGCTCA |
| 661 | GGGCCAAGAA CAGTTGGAAC AGCAGAATAT GGGCCAAACA GGATATCTGT GGTAAGCAGT |
| 721 | TCCTGCCCCG GCTCAGGGCC AAGAACAGAT GGTCCCCAGA TGCGGTCCCG CCCTCAGCAG |
| 781 | TTTCTAGAGA ACCATCAGAT GTTTCCAGGG TGCCCCAAGG ACCTGAAATG ACCCTGTGCC |
| 841 | TTATTTGAAC TAACCAATCA GTTCGCTTCT CGCTTCTGTT CGCGCGCTTC TGCTCCCCGA |
| 901 | GCTCTATATA AGCAGAGCTC GTTTAGTGAA CCGTCAGATC GCCTGGAGAC GCCATCCACG |
| 961 | CTGTTTTGAC TTCCATAGAA GGATCTCGAG GCCACCATGG TGAGCAAGGG CGAGGAGCTG |
| 1021 | TTCACCGGGG TGGTGCCCAT CCTGGTCGAG CTGGACGGCG ACGTAAACGG CCACAAGTTC |
| 1081 | AGCGTGTCCG GCGAGGGCGA GGGCGATGCC ACCTACGGCA AGCTGACCCT GAAGTTCATC |
| 1141 | TGCACCACCG GCAAGCTGCC CGTGCCCTGG CCCACCCTCG TGACCACCCT GACCTACGGC |
| 1201 | GTGCAGTGCT TCAGCCGCTA CCCCGACCAC ATGAAGCAGC ACGACTTCTT CAAGTCCGCC |
| 1261 | ATGCCCGAAG GCTACGTCCA GGAGCGCACC ATCTTCTTCA AGGACGACGG CAACTACAAG |
| 1321 | ACCCGCGCCG AGGTGAAGTT CGAGGGCGAC ACCCTGGTGA ACCGCATCGA GCTGAAGGGC |
| 1381 | ATCGACTTCA AGGAGGACGG CAACATCCTG GGGCACAAGC TGGAGTACAA CTACAACAGC |
| 1441 | CACAACGTCT ATATCATGGC CGACAAGCAG AAGAACGGCA TCAAGGTGAA CTTCAAGATC |
| 1501 | CGCCACAACA TCGAGGACGG CAGCGTGCAG CTCGCCGACC ACTACCAGCA GAACACCCCC |
| 1561 | ATCGGCGACG GCCCCGTGCT GCTGCCCGAC AACCACTACC TGAGCACCCA GTCCGCCCTG |
| 1621 | AGCAAAGACC CCAACGAGAA GCGCGATCAC ATGGTCCTGC TGGAGTTCGT GACCGCCGCC |
| 1681 | GGGATCACTC TCGGCATGGA CGAGCTGTAC AAGTAAACTA GTGTCGACTG CTTTATTTGT |
| 1741 | GAAATTTGTG ATGCTATTGC TTTATTTGTA ACCATTATAA GCTGCAATAA ACAAGTTAAC |
| 1801 | AACAACAATT GCATTCATTT TATGTTTCAG GTTCAGGGGG AGGTGTGGGA GGTTTTTTAA |
| 1861 | AACGTACATC ACTCGCTCCC CCATTCCATC CTCCACCACT CCAAGCCCCT CTGCAAGAAG |
| 1921 | CAGCCCCGAC CAAAGCCTCA AGAGCTCCAG CCCTCACAGC AGCCCTGGGA ATACGGTGTC |
| 1981 | CCCTGTGGGC CCCGGCTCTC AAGAGCACCG GGACTCCTAC GCTTACTTGA ACGCGTCCTA |
| 2041 | CGGCACGGAA GGTTTGGGCT CCTACCCTGG CTACGCACCC CTGCCCCACC TCCCGCCAGC |
| 2101 | TTTCATCCCC TCGTACAACG CTCACTACCC CAAGTTCCTC TTGCCCCCCT ACGGCATGAA |
| 2161 | TTGTAATGGC CTGAGCGCTG TGAGCAGCAT GAATGGCATC AACAACTTTG GCCTCTTCCC |
| 2221 | GAGGCTGTGC CCTGTCTACA GCAATCTCCT CGGTGGGGGC ACTAGTCCAC TCCCTCTCTG |
| 2281 | CGCGCTCGCT CGCTCACTGA GGCCGGGCGA CCAAAGGTCG CCCGACGCCC GGGCTTTGCC |
| 2341 | CGGGCGGCCT CAGTGAGCGA GCGAGCGCGC AGAGAGGGAC AGATCCGGGC CCGCATGCGT |
| 2401 | CGACAATTCA CTGGCCGTCG TTTTACAACG TCGTGACTGG GAAAACCCTG GCGTTACCCA |
| 2461 | ACTTAATCGC CTTGCAGCAC ATCCCCCTTT CGCCAGCTGG CGTAATAGCG AAGAGGCCCG |
| 2521 | CACCGATCGC CCTTCCCAAC AGTTGCGCAG CCTGAATGGC GAATGGCGCC TGATGCGGTA |
| 2581 | TTTTCTCCTT ACGCATCTGT GCGGTATTTC ACACCGCATA TGGTGCACTC TCAGTACAAT |
| 2641 | CTGCTCTGAT GCCGCATAGT TAAGCCAGCC CCGACACCCG CCAACACCCG CTGACGCGCC |
| 2701 | CTGACGGGCT TGTCTGCTCC CGGCATCCGC TTACAGACAA GCTGTGACCG TCTCCGGGAG |
| 2761 | CTGCATGTGT CAGAGGTTTT CACCGTCATC ACCGAAACGC GCGAGACGAA AGGGCCTCGT |
| 2821 | GATACGCCTA TTTTTATAGG TTAATGTCAT GATAATAATG GTTTCTTAGA CGTCAGGTGG |
| 2881 | CACTTTTCGG GGAAATGTGC GCGGAACCCC TATTTGTTTA TTTTTCTAAA TACATTCAAA |
| 2941 | TATGTATCCG CTCATGAGAC AATAACCCTG ATAAATGCTT CAATAATATT GAAAAAGGAA |
| 3001 | GAGTATGAGT ATTCAACATT TCCGTGTCGC CCTTATTCCC TTTTTTGCGG CATTTTGCCT |
| 3061 | TCCTGTTTTT GCTCACCCAG AAACGCTGGT GAAAGTAAAA GATGCTGAAG ATCAGTTGGG |
| 3121 | TGCACGAGTG GGTTACATCG AACTGGATCT CAACAGCGGT AAGATCCTTG AGAGTTTTCG |
| 3181 | CCCCGAAGAA CGTTTTCCAA TGATGAGCAC TTTTAAAGTT CTGCTATGTG GCGCGGTATT |
| 3241 | ATCCCGTATT GACGCCGGGC AAGAGCAACT CGGTCGCCGC ATACACTATT CTCAGAATGA |
| 3301 | CTTGGTTGAG TACTCACCAG TCACAGAAAA GCATCTTACG GATGGCATGA CAGTAAGAGA |
| 3361 | ATTATGCAGT GCTGCCATAA CCATGAGTGA TAACACTGCG GCCAACTTAC TTCTGACAAC |
| 3421 | GATCGGAGGA CCGAAGGAGC TAACCGCTTT TTTGCACAAC ATGGGGGATC ATGTAACTCG |
| 3481 | CCTTGATCGT TGGGAACCGG AGCTGAATGA AGCCATACCA AACGACGAGC GTGACACCAC |
| 3541 | GATGCCTGTA GCAATGGCAA CAACGTTGCG CAAACTATTA ACTGGCGAAC TACTTACTCT |
| 3601 | AGCTTCCCGG CAACAATTAA TAGACTGGAT GGAGGCGGAT AAAGTTGCAG GACCACTTCT |
| 3661 | GCGCTCGGCC CTTCCGGCTG GCTGGTTTAT TGCTGATAAA TCTGGAGCCG GTGAGCGTGG |
| 3721 | GTCTCGCGGT ATCATTGCAG CACTGGGGCC AGATGGTAAG CCCTCCCGTA TCGTAGTTAT |
| 3781 | CTACACGACG GGGAGTCAGG CAACTATGGA TGAACGAAAT AGACAGATCG CTGAGATAGG |
| 3841 | TGCCTCACTG ATTAAGCATT GGTAACTGTC AGACCAAGTT TACTCATATA TACTTTAGAT |
| 3901 | TGATTTAAAA CTTCATTTTT AATTTAAAAG GATCTAGGTG AAGATCCTTT TTGATAATCT |
| 3961 | CATGACCAAA ATCCCTTAAC GTGAGTTTTC GTTCCACTGA GCGTCAGACC CCGTAGAAAA |
| 4021 | GATCAAAGGA TCTTCTTGAG ATCCTTTTTT TCTGCGCGTA ATCTGCTGCT TGCAAACAAA |
| 4081 | AAAACCACCG CTACCAGCGG TGGTTTGTTT GCCGGATCAA GAGCTACCAA CTCTTTTTCC |
| 4141 | GAAGGTAACT GGCTTCAGCA GAGCGCAGAT ACCAAATACT GTTCTTCTAG TGTAGCCGTA |
| 4201 | GTTAGGCCAC CACTTCAAGA ACTCTGTAGC ACCGCCTACA TACCTCGCTC TGCTAATCCT |
| 4261 | GTTACCAGTG GCTGCTGCCA GTGGCGATAA GTCGTGTCTT ACCGGGTTGG ACTCAAGACG |
| 4321 | ATAGTTACCG GATAAGGCGC AGCGGTCGGG CTGAACGGGG GGTTCGTGCA CACAGCCCAG |
| 4381 | CTTGGAGCGA ACGACCTACA CCGAACTGAG ATACCTACAG CGTGAGCTAT GAGAAAGCGC |
| 4441 | CACGCTTCCC GAAGGGAGAA AGGCGGACAG GTATCCGGTA AGCGGCAGGG TCGGAACAGG |
| 4501 | AGAGCGCACG AGGGAGCTTC CAGGGGGAAA CGCCTGGTAT CTTTATAGTC CTGTCGGGTT |
| 4561 | TCGCCACCTC TGACTTGAGC GTCGATTTTT GTGATGCTCG TCAGGGGGGC GGAGCCTATG |
| 4621 | GAAAAACGCC AGCAACGCGG CCTTTTTACG GTTCCTGGCC TTTTGCTGGC CTTTTGCTCA |
| 4681 | CATGTTCTTT CCTGCGTTAT CCCCTGATTC TGTGGATAAC CGTATTACCG CCTTTGAGTG |
| 4741 | AGCTGATACC GCTCGCCGCA GCCGAACGAC CGAGCGCAGC GAGTCAGTGA GCGAGGAAGC |
| 4801 | GGAAGAGCGC CCAATACGCA AACCGCCTCT CCCCGCGCGT TGGCCGATTC ATTAATGCAG |
| 4861 | CTGGCACGAC AGGTTTCCCG ACTGGAAAGC GGGCAGTGAG CGCAACGCAA TTAATGTGAG |
| 4921 | TTAGCTCACT CATTAGGCAC CCCAGGCTTT ACACTTTATG CTTCCGGCTC GTATGTTGTG |
| 4981 | TGGAATTGTG AGCGGATAAC AATTTCACAC AGGAAACAGC TATGACCATG ATTACGCCAA |
| 5041 | GCTCTCGAGA TCTAGA |
| 1348_pAAV.Blimp.0.4 kb.MND.GFP.pA | |
| (SEQ ID NO: 16) | |
| 1 | CAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC ACGCGTGCCA |
| 181 | GCTGTTACTC AGGTTTTCTC AAGAAGGAGG AGCAACTTTG GCAGTTTTGC TTCAGTTCTC |
| 241 | TCTAGCCCTC TGTGTAATCG CCCCTTTTTC TTTATTTCAG CACAAACACA GAGCAGTCTA |
| 301 | AAGCAACCGA GCACTGAGAA AAATGAACTC TGCCCAAAGA ATGTCCCAAA GAGAGAGTAC |
| 361 | AGCGTGAAAG AAATCCTAAA ATTGGACTCC AACCCCTCCA AAGGAAAGGA CCTCTACCGT |
| 421 | TCTAACATTT CACCCCTCAC ATCAGAAAAG GACCTCGATG ACTTTAGAAG ACGTGGGAGC |
| 481 | CCCGAAATGC CCTTCTACCC TCGGGTCGTT TACCCCATCC GGGCCCCTCT GCCAGAAGAC |
| 541 | TTTTTGAAAG CTTCCCTGGC CTACGGGATC GAGAGAGAAC AGAGAAACAG GAGAATATGG |
| 601 | GCCAAACAGG ATATCTGTGG TAAGCAGTTC CTGCCCCGGC TCAGGGCCAA GAACAGTTGG |
| 661 | AACAGCAGAA TATGGGCCAA ACAGGATATC TGTGGTAAGC AGTTCCTGCC CCGGCTCAGG |
| 721 | GCCAAGAACA GATGGTCCCC AGATGCGGTC CCGCCCTCAG CAGTTTCTAG AGAACCATCA |
| 781 | GATGTTTCCA GGGTGCCCCA AGGACCTGAA ATGACCCTGT GCCTTATTTG AACTAACCAA |
| 841 | TCAGTTCGCT TCTCGCTTCT GTTCGCGCGC TTCTGCTCCC CGAGCTCTAT ATAAGCAGAG |
| 901 | CTCGTTTAGT GAACCGTCAG ATCGCCTGGA GACGCCATCC ACGCTGTTTT GACTTCCATA |
| 961 | GAAGGATCTC GAGGCCACCA TGGTGAGCAA GGGCGAGGAG CTGTTCACCG GGGTGGTGCC |
| 1021 | CATCCTGGTC GAGCTGGACG GCGACGTAAA CGGCCACAAG TTCAGCGTGT CCGGCGAGGG |
| 1081 | CGAGGGCGAT GCCACCTACG GCAAGCTGAC CCTGAAGTTC ATCTGCACCA CCGGCAAGCT |
| 1141 | GCCCGTGCCC TGGCCCACCC TCGTGACCAC CCTGACCTAC GGCGTGCAGT GCTTCAGCCG |
| 1201 | CTACCCCGAC CACATGAAGC AGCACGACTT CTTCAAGTCC GCCATGCCCG AAGGCTACGT |
| 1261 | CCAGGAGCGC ACCATCTTCT TCAAGGACGA CGGCAACTAC AAGACCCGCG CCGAGGTGAA |
| 1321 | GTTCGAGGGC GACACCCTGG TGAACCGCAT CGAGCTGAAG GGCATCGACT TCAAGGAGGA |
| 1381 | CGGCAACATC CTGGGGCACA AGCTGGAGTA CAACTACAAC AGCCACAACG TCTATATCAT |
| 1441 | GGCCGACAAG CAGAAGAACG GCATCAAGGT GAACTTCAAG ATCCGCCACA ACATCGAGGA |
| 1501 | CGGCAGCGTG CAGCTCGCCG ACCACTACCA GCAGAACACC CCCATCGGCG ACGGCCCCGT |
| 1561 | GCTGCTGCCC GACAACCACT ACCTGAGCAC CCAGTCCGCC CTGAGCAAAG ACCCCAACGA |
| 1621 | GAAGCGCGAT CACATGGTCC TGCTGGAGTT CGTGACCGCC GCCGGGATCA CTCTCGGCAT |
| 1681 | GGACGAGCTG TACAAGTAAA CTAGTGTCGA CTGCTTTATT TGTGAAATTT GTGATGCTAT |
| 1741 | TGCTTTATTT GTAACCATTA TAAGCTGCAA TAAACAAGTT AACAACAACA ATTGCATTCA |
| 1801 | TTTTATGTTT CAGGTTCAGG GGGAGGTGTG GGAGGTTTTT TAAAACGTAC ATCACTCGCT |
| 1861 | CCCCCATTCC ATCCTCCACC ACTCCAAGCC CCTCTGCAAG AAGCAGCCCC GACCAAAGCC |
| 1921 | TCAAGAGCTC CAGCCCTCAC AGCAGCCCTG GGAATACGGT GTCCCCTGTG GGCCCCGGCT |
| 1981 | CTCAAGAGCA CCGGGACTCC TACGCTTACT TGAACGCGTC CTACGGCACG GAAGGTTTGG |
| 2041 | GCTCCTACCC TGGCTACGCA CCCCTGCCCC ACCTCCCGCC AGCTTTCATC CCCTCGTACA |
| 2101 | ACGCTCACTA CCCCAAGTTC CTCTTGCCCC CCTACGGCAT GAATTGTAAT GGCCTGAGCG |
| 2161 | CTGTGAGCAG CATGAATGGC ATCAACAACT TTGGCCTCTT CCCGAGGCTG TGCCCTGTCT |
| 2221 | ACAGCAATCT CCTCGGTGGG GGCATCTAGA GTAGATAAGT AGCATGGCGG GTTAATCATT |
| 2281 | AACTACAAGG AACCCCTAGT GATGGAGTTG GCCACTCCCT CTCTGCGCGC TCGCTCGCTC |
| 2341 | ACTGAGGCCG GGCGACCAAA GGTCGCCCGA CGCCCGGGCT TTGCCCGGGC GGCCTCAGTG |
| 2401 | AGCGAGCGAG CGCGCCAGCT GGCGTAATAG CGAAGAGGCC CGCACCGATC GCCCTTCCCA |
| 2461 | ACAGTTGCGC AGCCTGAATG GCGAATGGCG ATTCCGTTGC AATGGCTGGC GGTAATATTG |
| 2521 | TTCTGGATAT TACCAGCAAG GCCGATAGTT TGAGTTCTTC TACTCAGGCA AGTGATGTTA |
| 2581 | TTACTAATCA AAGAAGTATT GCGACAACGG TTAATTTGCG TGATGGACAG ACTCTTTTAC |
| 2641 | TCGGTGGCCT CACTGATTAT AAAAACACTT CTCAGGATTC TGGCGTACCG TTCCTGTCTA |
| 2701 | AAATCCCTTT AATCGGCCTC CTGTTTAGCT CCCGCTCTGA TTCTAACGAG GAAAGCACGT |
| 2761 | TATACGTGCT CGTCAAAGCA ACCATAGTAC GCGCCCTGTA GCGGCGCATT AAGCGCGGCG |
| 2821 | GGTGTGGTGG TTACGCGCAG CGTGACCGCT ACACTTGCCA GCGCCCTAGC GCCCGCTCCT |
| 2881 | TTCGCTTTCT TCCCTTCCTT TCTCGCCACG TTCGCCGGCT TTCCCCGTCA AGCTCTAAAT |
| 2941 | CGGGGGCTCC CTTTAGGGTT CCGATTTAGT GCTTTACGGC ACCTCGACCC CAAAAAACTT |
| 3001 | GATTAGGGTG ATGGTTCACG TAGTGGGCCA TCGCCCTGAT AGACGGTTTT TCGCCCTTTG |
| 3061 | ACGTTGGAGT CCACGTTCTT TAATAGTGGA CTCTTGTTCC AAACTGGAAC AACACTCAAC |
| 3121 | CCTATCTCGG TCTATTCTTT TGATTTATAA GGGATTTTGC CGATTTCGGC CTATTGGTTA |
| 3181 | AAAAATGAGC TGATTTAACA AAAATTTAAC GCGAATTTTA ACAAAATATT AACGTTTACA |
| 3241 | ATTTAAATAT TTGCTTATAC AATCTTCCTG TTTTTGGGGC TTTTCTGATT ATCAACCGGG |
| 3301 | GTACATATGA TTGACATGCT AGTTTTACGA TTACCGTTCA TCGATTCTCT TGTTTGCTCC |
| 3361 | AGACTCTCAG GCAATGACCT GATAGCCTTT GTAGAGACCT CTCAAAAATA GCTACCCTCT |
| 3421 | CCGGCATGAA TTTATCAGCT AGAACGGTTG AATATCATAT TGATGGTGAT TTGACTGTCT |
| 3481 | CCGGCCTTTC TCACCCGTTT GAATCTTTAC CTACACATTA CTCAGGCATT GCATTTAAAA |
| 3541 | TATATGAGGG TTCTAAAAAT TTTTATCCTT GCGTTGAAAT AAAGGCTTCT CCCGCAAAAG |
| 3601 | TATTACAGGG TCATAATGTT TTTGGTACAA CCGATTTAGC TTTATGCTCT GAGGCTTTAT |
| 3661 | TGCTTAATTT TGCTAATTCT TTGCCTTGCC TGTATGATTT ATTGGATGTT GGAATCGCCT |
| 3721 | GATGCGGTAT TTTCTCCTTA CGCATCTGTG CGGTATTTCA CACCGCATAT GGTGCACTCT |
| 3781 | CAGTACAATC TGCTCTGATG CCGCATAGTT AAGCCAGCCC CGACACCCGC CAACACCCGC |
| 3841 | TGACGCGCCC TGACGGGCTT GTCTGCTCCC GGCATCCGCT TACAGACAAG CTGTGACCGT |
| 3901 | CTCCGGGAGC TGCATGTGTC AGAGGTTTTC ACCGTCATCA CCGAAACGCG CGAGACGAAA |
| 3961 | GGGCCTCGTG ATACGCCTAT TTTTATAGGT TAATGTCATG ATAATAATGG TTTCTTAGAC |
| 4021 | GTCAGGTGGC ACTTTTCGGG GAAATGTGCG CGGAACCCCT ATTTGTTTAT TTTTCTAAAT |
| 4081 | ACATTCAAAT ATGTATCCGC TCATGAGACA ATAACCCTGA TAAATGCTTC AATAATATTG |
| 4141 | AAAAAGGAAG AGTATGAGTA TTCAACATTT CCGTGTCGCC CTTATTCCCT TTTTTGCGGC |
| 4201 | ATTTTGCCTT CCTGTTTTTG CTCACCCAGA AACGCTGGTG AAAGTAAAAG ATGCTGAAGA |
| 4261 | TCAGTTGGGT GCACGAGTGG GTTACATCGA ACTGGATCTC AACAGCGGTA AGATCCTTGA |
| 4321 | GAGTTTTCGC CCCGAAGAAC GTTTTCCAAT GATGAGCACT TTTAAAGTTC TGCTATGTGG |
| 4381 | CGCGGTATTA TCCCGTATTG ACGCCGGGCA AGAGCAACTC GGTCGCCGCA TACACTATTC |
| 4441 | TCAGAATGAC TTGGTTGAGT ACTCACCAGT CACAGAAAAG CATCTTACGG ATGGCATGAC |
| 4501 | AGTAAGAGAA TTATGCAGTG CTGCCATAAC CATGAGTGAT AACACTGCGG CCAACTTACT |
| 4561 | TCTGACAACG ATCGGAGGAC CGAAGGAGCT AACCGCTTTT TTGCACAACA TGGGGGATCA |
| 4621 | TGTAACTCGC CTTGATCGTT GGGAACCGGA GCTGAATGAA GCCATACCAA ACGACGAGCG |
| 4681 | TGACACCACG ATGCCTGTAG CAATGGCAAC AACGTTGCGC AAACTATTAA CTGGCGAACT |
| 4741 | ACTTACTCTA GCTTCCCGGC AACAATTAAT AGACTGGATG GAGGCGGATA AAGTTGCAGG |
| 4801 | ACCACTTCTG CGCTCGGCCC TTCCGGCTGG CTGGTTTATT GCTGATAAAT CTGGAGCCGG |
| 4861 | TGAGCGTGGG TCTCGCGGTA TCATTGCAGC ACTGGGGCCA GATGGTAAGC CCTCCCGTAT |
| 4921 | CGTAGTTATC TACACGACGG GGAGTCAGGC AACTATGGAT GAACGAAATA GACAGATCGC |
| 4981 | TGAGATAGGT GCCTCACTGA TTAAGCATTG GTAACTGTCA GACCAAGTTT ACTCATATAT |
| 5041 | ACTTTAGATT GATTTAAAAC TTCATTTTTA ATTTAAAAGG ATCTAGGTGA AGATCCTTTT |
| 5101 | TGATAATCTC ATGACCAAAA TCCCTTAACG TGAGTTTTCG TTCCACTGAG CGTCAGACCC |
| 5161 | CGTAGAAAAG ATCAAAGGAT CTTCTTGAGA TCCTTTTTTT CTGCGCGTAA TCTGCTGCTT |
| 5221 | GCAAACAAAA AAACCACCGC TACCAGCGGT GGTTTGTTTG CCGGATCAAG AGCTACCAAC |
| 5281 | TCTTTTTCCG AAGGTAACTG GCTTCAGCAG AGCGCAGATA CCAAATACTG TCCTTCTAGT |
| 5341 | GTAGCCGTAG TTAGGCCACC ACTTCAAGAA CTCTGTAGCA CCGCCTACAT ACCTCGCTCT |
| 5401 | GCTAATCCTG TTACCAGTGG CTGCTGCCAG TGGCGATAAG TCGTGTCTTA CCGGGTTGGA |
| 5461 | CTCAAGACGA TAGTTACCGG ATAAGGCGCA GCGGTCGGGC TGAACGGGGG GTTCGTGCAC |
| 5521 | ACAGCCCAGC TTGGAGCGAA CGACCTACAC CGAACTGAGA TACCTACAGC GTGAGCTATG |
| 5581 | AGAAAGCGCC ACGCTTCCCG AAGGGAGAAA GGCGGACAGG TATCCGGTAA GCGGCAGGGT |
| 5641 | CGGAACAGGA GAGCGCACGA GGGAGCTTCC AGGGGGAAAC GCCTGGTATC TTTATAGTCC |
| 5701 | TGTCGGGTTT CGCCACCTCT GACTTGAGCG TCGATTTTTG TGATGCTCGT CAGGGGGGCG |
| 5761 | GAGCCTATGG AAAAACGCCA GCAACGCGGC CTTTTTACGG TTCCTGGCCT TTTGCTGGCC |
| 5821 | TTTTGCTCAC ATGTTCTTTC CTGCGTTATC CCCTGATTCT GTGGATAACC GTATTACCGC |
| 5881 | CTTTGAGTGA GCTGATACCG CTCGCCGCAG CCGAACGACC GAGCGCAGCG AGTCAGTGAG |
| 5941 | CGAGGAAGCG GAAGAGCGCC CAATACGCAA ACCGCCTCTC CCCGCGCGTT GGCCGATTCA |
| 6001 | TTAATG |
| 1361_pAAV.Blimp1.1.0 kb.MND.GFP.pA | |
| (SEQ ID NO: 17) | |
| 1 | CAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC ACGCGTGGTA |
| 181 | AACCATGAAC ATCAGAAAGA CTTTTATTAA CCTATGACAG GGTCCCCACC CCAGTATTTT |
| 241 | TCCACTCCAT TAAAATGGAA GTTTTTTTTT TTTTTTTCTT TTTTGAGACA GAGTTTTGCT |
| 301 | CTTGTTGCCC AGTCTGGAGT GCAATGGCAC AATCTCGGCT CACCACAACC TCCACCTCCC |
| 361 | AGATTCAAGC GATTCTTCTG CCTCAGCCTC CCAAGTAGCT GGGATTACAG GTGTGCGCCA |
| 421 | CCACGCCCAG CTAATTTTGT ATTTTTAGTA GAGATGGGGT TTCTCCATGT TGGTCAGGCT |
| 481 | GGTCTCGAAC TTCCGACCTC AGGTGATCCG CCCACCTCGG CCTCCCAAAG TGCTGGGATT |
| 541 | ACAGGCAAGA GCCACTGCAT CCAGCTTAGG CTATCTTACT CCAGCCTAAA CAGCAATTTT |
| 601 | CTATCATAAG GTCTGTACTA ATGAAAACAG AATCACCCAA GGCTGCTGTT TGTTCTGTCT |
| 661 | GTGCTGCCAT TGTCCGCATT TTGCTGAGGA GGAAACGGAA CTGCACTTTT GAGTGAGTGG |
| 721 | CCCAGAGCCT TCTAGAATGA GAGTGCGTTG GAAGCCAGAT ATGTGGCGAT TGTGTCGCCA |
| 781 | GCTGTTACTC AGGTTTTCTC AAGAAGGAGG AGCAACTTTG GCAGTTTTGC TTCAGTTCTC |
| 841 | TCTAGCCCTC TGTGTAATCG CCCCTTTTTC TTTATTTCAG CACAAACACA GAGCAGTCTA |
| 901 | AAGCAACCGA GCACTGAGAA AAATGAACTC TGCCCAAAGA ATGTCCCAAA GAGAGAGTAC |
| 961 | AGCGTGAAAG AAATCCTAAA ATTGGACTCC AACCCCTCCA AAGGAAAGGA CCTCTACCGT |
| 1021 | TCTAACATTT CACCCCTCAC ATCAGAAAAG GACCTCGATG ACTTTAGAAG ACGTGGGAGC |
| 1081 | CCCGAAATGC CCTTCTACCC TCGGGTCGTT TACCCCATCC GGGCCCCTCT GCCAGAAGAC |
| 1141 | TTTTTGAAAG CTTCCCTGGC CTACGGGATC GAGAGAGAAC AGAGAAACAG GAGAATATGG |
| 1201 | GCCAAACAGG ATATCTGTGG TAAGCAGTTC CTGCCCCGGC TCAGGGCCAA GAACAGTTGG |
| 1261 | AACAGCAGAA TATGGGCCAA ACAGGATATC TGTGGTAAGC AGTTCCTGCC CCGGCTCAGG |
| 1321 | GCCAAGAACA GATGGTCCCC AGATGCGGTC CCGCCCTCAG CAGTTTCTAG AGAACCATCA |
| 1381 | GATGTTTCCA GGGTGCCCCA AGGACCTGAA ATGACCCTGT GCCTTATTTG AACTAACCAA |
| 1441 | TCAGTTCGCT TCTCGCTTCT GTTCGCGCGC TTCTGCTCCC CGAGCTCTAT ATAAGCAGAG |
| 1501 | CTCGTTTAGT GAACCGTCAG ATCGCCTGGA GACGCCATCC ACGCTGTTTT GACTTCCATA |
| 1561 | GAAGGATCTC GAGGCCACCA TGGTGAGCAA GGGCGAGGAG CTGTTCACCG GGGTGGTGCC |
| 1621 | CATCCTGGTC GAGCTGGACG GCGACGTAAA CGGCCACAAG TTCAGCGTGT CCGGCGAGGG |
| 1681 | CGAGGGCGAT GCCACCTACG GCAAGCTGAC CCTGAAGTTC ATCTGCACCA CCGGCAAGCT |
| 1741 | GCCCGTGCCC TGGCCCACCC TCGTGACCAC CCTGACCTAC GGCGTGCAGT GCTTCAGCCG |
| 1801 | CTACCCCGAC CACATGAAGC AGCACGACTT CTTCAAGTCC GCCATGCCCG AAGGCTACGT |
| 1861 | CCAGGAGCGC ACCATCTTCT TCAAGGACGA CGGCAACTAC AAGACCCGCG CCGAGGTGAA |
| 1921 | GTTCGAGGGC GACACCCTGG TGAACCGCAT CGAGCTGAAG GGCATCGACT TCAAGGAGGA |
| 1981 | CGGCAACATC CTGGGGCACA AGCTGGAGTA CAACTACAAC AGCCACAACG TCTATATCAT |
| 2041 | GGCCGACAAG CAGAAGAACG GCATCAAGGT GAACTTCAAG ATCCGCCACA ACATCGAGGA |
| 2101 | CGGCAGCGTG CAGCTCGCCG ACCACTACCA GCAGAACACC CCCATCGGCG ACGGCCCCGT |
| 2161 | GCTGCTGCCC GACAACCACT ACCTGAGCAC CCAGTCCGCC CTGAGCAAAG ACCCCAACGA |
| 2221 | GAAGCGCGAT CACATGGTCC TGCTGGAGTT CGTGACCGCC GCCGGGATCA CTCTCGGCAT |
| 2281 | GGACGAGCTG TACAAGTAAA CTAGTGTCGA CTGCTTTATT TGTGAAATTT GTGATGCTAT |
| 2341 | TGCTTTATTT GTAACCATTA TAAGCTGCAA TAAACAAGTT AACAACAACA ATTGCATTCA |
| 2401 | TTTTATGTTT CAGGTTCAGG GGGAGGTGTG GGAGGTTTTT TAAAACGTAC ATCACTCGCT |
| 2461 | CCCCCATTCC ATCCTCCACC ACTCCAAGCC CCTCTGCAAG AAGCAGCCCC GACCAAAGCC |
| 2521 | TCAAGAGCTC CAGCCCTCAC AGCAGCCCTG GGAATACGGT GTCCCCTGTG GGCCCCGGCT |
| 2581 | CTCAAGAGCA CCGGGACTCC TACGCTTACT TGAACGCGTC CTACGGCACG GAAGGTTTGG |
| 2641 | GCTCCTACCC TGGCTACGCA CCCCTGCCCC ACCTCCCGCC AGCTTTCATC CCCTCGTACA |
| 2701 | ACGCTCACTA CCCCAAGTTC CTCTTGCCCC CCTACGGCAT GAATTGTAAT GGCCTGAGCG |
| 2761 | CTGTGAGCAG CATGAATGGC ATCAACAACT TTGGCCTCTT CCCGAGGCTG TGCCCTGTCT |
| 2821 | ACAGCAATCT CCTCGGTGGG GGCAGCCTGC CCCACCCCAT GCTCAACCCC ACTTCTCTCC |
| 2881 | CGAGCTCGCT GCCCTCAGAT GGAGCCCGGA GGTTGCTCCA GCCGGAGCAT CCCAGGGAGG |
| 2941 | TGCTTGTCCC GGCGCCCCAC AGTGCCTTCT CCTTTACCGG GGCCGCCGCC AGCATGAAGG |
| 3001 | ACAAGGCCTG TAGCCCCACA AGCGGGTCTC CCACGGCGGG AACAGCCGCC ACGGCAGAAC |
| 3061 | ATGTGGTGCA GCCCAAAGCT ACCTCAGCAG CGATGGCAGC CCCCAGCAGC GACGAAGCCA |
| 3121 | TGAATCTCAT TAAAAACAAA AGAAACATGA CCGGCTACAA GACCCTTCCC TACCCGCTGA |
| 3181 | AGAAGCAGAA CGGCAAGATC AAGTACGAAT GCAACGTTTG CGCCAAGACT TTCGGCCAGC |
| 3241 | TCTCCAATCT GAAGGTAGGC CTTGAGAGAG AGCAGTCCAA GGGGCTGTGA GTGCATGCTT |
| 3301 | GTGTTTGTAT TTAGCTTGCT TTCCATGGGG TATCGATTGC ATTTGCAGTA GTATGAGCCC |
| 3361 | CCGGTTGGGG ATAGTGGGTA TGGATTCCGC CTGGCTTTTG CCACTTCTAG CTCTTTGACT |
| 3421 | TTGGACAAGT GACTTCCCTT CTCCTCTAGA GTAGATAAGT AGCATGGCGG GTTAATCATT |
| 3481 | AACTACAAGG AACCCCTAGT GATGGAGTTG GCCACTCCCT CTCTGCGCGC TCGCTCGCTC |
| 3541 | ACTGAGGCCG GGCGACCAAA GGTCGCCCGA CGCCCGGGCT TTGCCCGGGC GGCCTCAGTG |
| 3601 | AGCGAGCGAG CGCGCCAGCT GGCGTAATAG CGAAGAGGCC CGCACCGATC GCCCTTCCCA |
| 3661 | ACAGTTGCGC AGCCTGAATG GCGAATGGCG ATTCCGTTGC AATGGCTGGC GGTAATATTG |
| 3721 | TTCTGGATAT TACCAGCAAG GCCGATAGTT TGAGTTCTTC TACTCAGGCA AGTGATGTTA |
| 3781 | TTACTAATCA AAGAAGTATT GCGACAACGG TTAATTTGCG TGATGGACAG ACTCTTTTAC |
| 3841 | TCGGTGGCCT CACTGATTAT AAAAACACTT CTCAGGATTC TGGCGTACCG TTCCTGTCTA |
| 3901 | AAATCCCTTT AATCGGCCTC CTGTTTAGCT CCCGCTCTGA TTCTAACGAG GAAAGCACGT |
| 3961 | TATACGTGCT CGTCAAAGCA ACCATAGTAC GCGCCCTGTA GCGGCGCATT AAGCGCGGCG |
| 4021 | GGTGTGGTGG TTACGCGCAG CGTGACCGCT ACACTTGCCA GCGCCCTAGC GCCCGCTCCT |
| 4081 | TTCGCTTTCT TCCCTTCCTT TCTCGCCACG TTCGCCGGCT TTCCCCGTCA AGCTCTAAAT |
| 4141 | CGGGGGCTCC CTTTAGGGTT CCGATTTAGT GCTTTACGGC ACCTCGACCC CAAAAAACTT |
| 4201 | GATTAGGGTG ATGGTTCACG TAGTGGGCCA TCGCCCTGAT AGACGGTTTT TCGCCCTTTG |
| 4261 | ACGTTGGAGT CCACGTTCTT TAATAGTGGA CTCTTGTTCC AAACTGGAAC AACACTCAAC |
| 4321 | CCTATCTCGG TCTATTCTTT TGATTTATAA GGGATTTTGC CGATTTCGGC CTATTGGTTA |
| 4381 | AAAAATGAGC TGATTTAACA AAAATTTAAC GCGAATTTTA ACAAAATATT AACGTTTACA |
| 4441 | ATTTAAATAT TTGCTTATAC AATCTTCCTG TTTTTGGGGC TTTTCTGATT ATCAACCGGG |
| 4501 | GTACATATGA TTGACATGCT AGTTTTACGA TTACCGTTCA TCGATTCTCT TGTTTGCTCC |
| 4561 | AGACTCTCAG GCAATGACCT GATAGCCTTT GTAGAGACCT CTCAAAAATA GCTACCCTCT |
| 4621 | CCGGCATGAA TTTATCAGCT AGAACGGTTG AATATCATAT TGATGGTGAT TTGACTGTCT |
| 4681 | CCGGCCTTTC TCACCCGTTT GAATCTTTAC CTACACATTA CTCAGGCATT GCATTTAAAA |
| 4741 | TATATGAGGG TTCTAAAAAT TTTTATCCTT GCGTTGAAAT AAAGGCTTCT CCCGCAAAAG |
| 4801 | TATTACAGGG TCATAATGTT TTTGGTACAA CCGATTTAGC TTTATGCTCT GAGGCTTTAT |
| 4861 | TGCTTAATTT TGCTAATTCT TTGCCTTGCC TGTATGATTT ATTGGATGTT GGAATCGCCT |
| 4921 | GATGCGGTAT TTTCTCCTTA CGCATCTGTG CGGTATTTCA CACCGCATAT GGTGCACTCT |
| 4981 | CAGTACAATC TGCTCTGATG CCGCATAGTT AAGCCAGCCC CGACACCCGC CAACACCCGC |
| 5041 | TGACGCGCCC TGACGGGCTT GTCTGCTCCC GGCATCCGCT TACAGACAAG CTGTGACCGT |
| 5101 | CTCCGGGAGC TGCATGTGTC AGAGGTTTTC ACCGTCATCA CCGAAACGCG CGAGACGAAA |
| 5161 | GGGCCTCGTG ATACGCCTAT TTTTATAGGT TAATGTCATG ATAATAATGG TTTCTTAGAC |
| 5221 | GTCAGGTGGC ACTTTTCGGG GAAATGTGCG CGGAACCCCT ATTTGTTTAT TTTTCTAAAT |
| 5281 | ACATTCAAAT ATGTATCCGC TCATGAGACA ATAACCCTGA TAAATGCTTC AATAATATTG |
| 5341 | AAAAAGGAAG AGTATGAGTA TTCAACATTT CCGTGTCGCC CTTATTCCCT TTTTTGCGGC |
| 5401 | ATTTTGCCTT CCTGTTTTTG CTCACCCAGA AACGCTGGTG AAAGTAAAAG ATGCTGAAGA |
| 5461 | TCAGTTGGGT GCACGAGTGG GTTACATCGA ACTGGATCTC AACAGCGGTA AGATCCTTGA |
| 5521 | GAGTTTTCGC CCCGAAGAAC GTTTTCCAAT GATGAGCACT TTTAAAGTTC TGCTATGTGG |
| 5581 | CGCGGTATTA TCCCGTATTG ACGCCGGGCA AGAGCAACTC GGTCGCCGCA TACACTATTC |
| 5641 | TCAGAATGAC TTGGTTGAGT ACTCACCAGT CACAGAAAAG CATCTTACGG ATGGCATGAC |
| 5701 | AGTAAGAGAA TTATGCAGTG CTGCCATAAC CATGAGTGAT AACACTGCGG CCAACTTACT |
| 5761 | TCTGACAACG ATCGGAGGAC CGAAGGAGCT AACCGCTTTT TTGCACAACA TGGGGGATCA |
| 5821 | TGTAACTCGC CTTGATCGTT GGGAACCGGA GCTGAATGAA GCCATACCAA ACGACGAGCG |
| 5881 | TGACACCACG ATGCCTGTAG CAATGGCAAC AACGTTGCGC AAACTATTAA CTGGCGAACT |
| 5941 | ACTTACTCTA GCTTCCCGGC AACAATTAAT AGACTGGATG GAGGCGGATA AAGTTGCAGG |
| 6001 | ACCACTTCTG CGCTCGGCCC TTCCGGCTGG CTGGTTTATT GCTGATAAAT CTGGAGCCGG |
| 6061 | TGAGCGTGGG TCTCGCGGTA TCATTGCAGC ACTGGGGCCA GATGGTAAGC CCTCCCGTAT |
| 6121 | CGTAGTTATC TACACGACGG GGAGTCAGGC AACTATGGAT GAACGAAATA GACAGATCGC |
| 6181 | TGAGATAGGT GCCTCACTGA TTAAGCATTG GTAACTGTCA GACCAAGTTT ACTCATATAT |
| 6241 | ACTTTAGATT GATTTAAAAC TTCATTTTTA ATTTAAAAGG ATCTAGGTGA AGATCCTTTT |
| 6301 | TGATAATCTC ATGACCAAAA TCCCTTAACG TGAGTTTTCG TTCCACTGAG CGTCAGACCC |
| 6361 | CGTAGAAAAG ATCAAAGGAT CTTCTTGAGA TCCTTTTTTT CTGCGCGTAA TCTGCTGCTT |
| 6421 | GCAAACAAAA AAACCACCGC TACCAGCGGT GGTTTGTTTG CCGGATCAAG AGCTACCAAC |
| 6481 | TCTTTTTCCG AAGGTAACTG GCTTCAGCAG AGCGCAGATA CCAAATACTG TCCTTCTAGT |
| 6541 | GTAGCCGTAG TTAGGCCACC ACTTCAAGAA CTCTGTAGCA CCGCCTACAT ACCTCGCTCT |
| 6601 | GCTAATCCTG TTACCAGTGG CTGCTGCCAG TGGCGATAAG TCGTGTCTTA CCGGGTTGGA |
| 6661 | CTCAAGACGA TAGTTACCGG ATAAGGCGCA GCGGTCGGGC TGAACGGGGG GTTCGTGCAC |
| 6721 | ACAGCCCAGC TTGGAGCGAA CGACCTACAC CGAACTGAGA TACCTACAGC GTGAGCTATG |
| 6781 | AGAAAGCGCC ACGCTTCCCG AAGGGAGAAA GGCGGACAGG TATCCGGTAA GCGGCAGGGT |
| 6841 | CGGAACAGGA GAGCGCACGA GGGAGCTTCC AGGGGGAAAC GCCTGGTATC TTTATAGTCC |
| 6901 | TGTCGGGTTT CGCCACCTCT GACTTGAGCG TCGATTTTTG TGATGCTCGT CAGGGGGGCG |
| 6961 | GAGCCTATGG AAAAACGCCA GCAACGCGGC CTTTTTACGG TTCCTGGCCT TTTGCTGGCC |
| 7021 | TTTTGCTCAC ATGTTCTTTC CTGCGTTATC CCCTGATTCT GTGGATAACC GTATTACCGC |
| 7081 | CTTTGAGTGA GCTGATACCG CTCGCCGCAG CCGAACGACC GAGCGCAGCG AGTCAGTGAG |
| 7141 | CGAGGAAGCG GAAGAGCGCC CAATACGCAA ACCGCCTCTC CCCGCGCGTT GGCCGATTCA |
| 7201 | TTAATG |
| 1366_CCR5.MND.BAFF_CRISPR.HR | |
| (SEQ ID NO: 18) | |
| 1 | CAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGCGGCC TCGGCCTCTG CATAAATAAA AAAAATTAGT CAGCCATGAG CTTGGACGCG |
| 241 | TTTGGTGTGG TGGCGCCTGT AGTCCCCAGC CACTTGGAGG GGTGAGGTGA GAGGATTGCT |
| 301 | TGAGCCCGGG ATGGTCCAGG CTGCAGTGAG CCATGATCGT GCCACTGCAC TCCAGCCTGG |
| 361 | GCGACAGAGT GAGACCCTGT CTCACAACAA CAACAACAAC AACAAAAAGG CTGAGCTGCA |
| 421 | CCATGCTTGA CCCAGTTTCT TAAAATTGTT GTCAAAGCTT CATTCACTCC ATGGTGCTAT |
| 481 | AGAGCACAAG ATTTTATTTG GTGAGATGGT GCTTTCATGA ATTCCCCCAA CAGAGCCAAG |
| 541 | CTCTCCATCT AGTGGACAGG GAAGCTAGCA GCAAACCTTC CCTTCACTAC AAAACTTCAT |
| 601 | TGCTTGGCCA AAAAGAGAGT TAATTCAATG TAGACATCTA TGTAGGCAAT TAAAAACCTA |
| 661 | TTGATGTATA AAACAGTTTG CATTCATGGA GGGCAACTAA ATACATTCTA GGACTTTATA |
| 721 | AAAGATCACT TTTTATTTAT GCACAGGGTG GAACAAGATG GATTATCAAG TGTCAAGTCC |
| 781 | AATCTATGAC ATCAATTATT ATACATCGGA GCCCTGCCAA AAAATCAATG TGAAGCAAAT |
| 841 | CGCAGCCCGC CTCCTGCCTC CGCTCTACTC ACTGGTGTTC ATCTTTGGTT TTGTGGGCAA |
| 901 | CATGCTGGTC ATCCTCATCC TGATAAACTG CAAAAGGCTG AAGAGCATGA CTGACATCTA |
| 961 | CCTGCTCAAC CTGGCCATCT CTGACCTGTT TTTCCTTCTT ACTGTCCCCT TCTGGGCTCA |
| 1021 | CTATGCTGCC GCCCAGTGGG ACTTTGGAAA TACAATGTGT CAACGAACAG AGAAACAGGA |
| 1081 | GAATATGGGC CAAACAGGAT ATCTGTGGTA AGCAGTTCCT GCCCCGGCTC AGGGCCAAGA |
| 1141 | ACAGTTGGAA CAGCAGAATA TGGGCCAAAC AGGATATCTG TGGTAAGCAG TTCCTGCCCC |
| 1201 | GGCTCAGGGC CAAGAACAGA TGGTCCCCAG ATGCGGTCCC GCCCTCAGCA GTTTCTAGAG |
| 1261 | AACCATCAGA TGTTTCCAGG GTGCCCCAAG GACCTGAAAT GACCCTGTGC CTTATTTGAA |
| 1321 | CTAACCAATC AGTTCGCTTC TCGCTTCTGT TCGCGCGCTT CTGCTCCCCG AGCTCTATAT |
| 1381 | AAGCAGAGCT CGTTTAGTGA ACCGTCAGAT CGCCTGGAGA CGCCATCCAC GCTGTTTTGA |
| 1441 | CTTCCATAGA AGGATCTCGA GGCCACCATG TACCGGATGC AGCTGCTGAG CTGCATCGCA |
| 1501 | CTGAGCCTGG CACTGGTGAC CAACAGCGCA GTGCAGGGAC CAGAGGAGAC CGTGACCCAG |
| 1561 | GACTGCCTGC AGCTGATCGC AGACAGCGAG ACCCCCACCA TCCAGAAGGG CAGCTACACC |
| 1621 | TTCGTGCCCT GGCTGCTGAG CTTCAAGCGG GGCAGCGCCC TGGAGGAGAA GGAGAACAAG |
| 1681 | ATTCTGGTGA AGGAGACCGG CTACTTCTTC ATCTACGGCC AGGTGCTGTA CACCGATAAG |
| 1741 | ACCTACGCCA TGGGCCACCT GATCCAGCGG AAGAAGGTGC ACGTGTTCGG CGACGAGCTG |
| 1801 | AGCCTGGTGA CCCTGTTCCG GTGCATCCAG AACATGCCCG AGACCCTGCC CAACAACAGC |
| 1861 | TGCTACAGCG CAGGAATCGC AAAGCTGGAG GAGGGCGACG AGCTGCAGCT GGCAATCCCC |
| 1921 | CGGGAGAACG CACAGATCAG CCTGGACGGC GACGTGACCT TCTTCGGCGC CCTGAAGCTC |
| 1981 | CTGTGAGTCG ACTGCTTTAT TTGTGAAATT TGTGATGCTA TTGCTTTATT TGTAACCATT |
| 2041 | ATAAGCTGCA ATAAACAAGT TAACAACAAC AATTGCATTC ATTTTATGTT TCAGGTTCAG |
| 2101 | GGGGAGGTGT GGGAGGTTTT TTAAACTCTA TTTTATAGGC TTCTTCTCTG GAATCTTCTT |
| 2161 | CATCATCCTC CTGACAATCG ATAGGTACCT GGCTGTCGTC CATGCTGTGT TTGCTTTAAA |
| 2221 | AGCCAGGACG GTCACCTTTG GGGTGGTGAC AAGTGTGATC ACTTGGGTGG TGGCTGTGTT |
| 2281 | TGCGTCTCTC CCAGGAATCA TCTTTACCAG ATCTCAAAAA GAAGGTCTTC ATTACACCTG |
| 2341 | CAGCTCTCAT TTTCCATACA GTCAGTATCA ATTCTGGAAG AATTTCCAGA CATTAAAGAT |
| 2401 | AGTCATCTTG GGGCTGGTCC TGCCGCTGCT TGTCATGGTC ATCTGCTACT CGGGAATCCT |
| 2461 | AAAAACTCTG CTTCGGTGTC GAAATGAGAA GAAGAGGCAC AGGGCTGTGA GGCTTATCTT |
| 2521 | CACCATCATG ATTGTTTATT TTCTCTTCTG GGCTCCCTAC AACATTGTCC TTCTCCTGAA |
| 2581 | CACCTTCCAG GAATTCTTTG GCCTGAATAA TTGCAGTAGC TCTAACAGGT TGGACCAAGC |
| 2641 | TATGCAGGTG ACAGAGACTC TTGGGATGAC GCACTGCTGC ATCAACCCCA TCATCTATGC |
| 2701 | CTTTGTCGGG GAGAAGTTCA GAAACTACCT CTTAGTCTTC TTCCAAAAGC ACATTGCCAA |
| 2761 | ACGCTTCTGC AAATGCTGTT CTATTTTCCA GCAAGAGGCT CCCGAGCGAG CAAGCTCAGT |
| 2821 | TTACACCCGA TCCACTGGGG AGCAGGAAAT ATCTGTGGGC TTGTGACACG GACTCAAGTG |
| 2881 | GGCTGGTGAC CCAGTCAGAG TTGTGCACAT GGCTTAGTTT TCATACACAC CGCGGTCTAG |
| 2941 | AGCATGGCTA CGTAGATAAG TAGCATGGCG GGTTAATCAT TAACTACAAG GAACCCCTAG |
| 3001 | TGATGGAGTT GGCCACTCCC TCTCTGCGCG CTCGCTCGCT CACTGAGGCC GGGCGACCAA |
| 3061 | AGGTCGCCCG ACGCCCGGGC TTTGCCCGGG CGGCCTCAGT GAGCGAGCGA GCGCGCCAGC |
| 3121 | TGGCGTAATA GCGAAGAGGC CCGCACCGAT CGCCCTTCCC AACAGTTGCG CAGCCTGAAT |
| 3181 | GGCGAATGGC GATTCCGTTG CAATGGCTGG CGGTAATATT GTTCTGGATA TTACCAGCAA |
| 3241 | GGCCGATAGT TTGAGTTCTT CTACTCAGGC AAGTGATGTT ATTACTAATC AAAGAAGTAT |
| 3301 | TGCGACAACG GTTAATTTGC GTGATGGACA GACTCTTTTA CTCGGTGGCC TCACTGATTA |
| 3361 | TAAAAACACT TCTCAGGATT CTGGCGTACC GTTCCTGTCT AAAATCCCTT TAATCGGCCT |
| 3421 | CCTGTTTAGC TCCCGCTCTG ATTCTAACGA GGAAAGCACG TTATACGTGC TCGTCAAAGC |
| 3481 | AACCATAGTA CGCGCCCTGT AGCGGCGCAT TAAGCGCGGC GGGTGTGGTG GTTACGCGCA |
| 3541 | GCGTGACCGC TACACTTGCC AGCGCCCTAG CGCCCGCTCC TTTCGCTTTC TTCCCTTCCT |
| 3601 | TTCTCGCCAC GTTCGCCGGC TTTCCCCGTC AAGCTCTAAA TCGGGGGCTC CCTTTAGGGT |
| 3661 | TCCGATTTAG TGCTTTACGG CACCTCGACC CCAAAAAACT TGATTAGGGT GATGGTTCAC |
| 3721 | GTAGTGGGCC ATCGCCCTGA TAGACGGTTT TTCGCCCTTT GACGTTGGAG TCCACGTTCT |
| 3781 | TTAATAGTGG ACTCTTGTTC CAAACTGGAA CAACACTCAA CCCTATCTCG GTCTATTCTT |
| 3841 | TTGATTTATA AGGGATTTTG CCGATTTCGG CCTATTGGTT AAAAAATGAG CTGATTTAAC |
| 3901 | AAAAATTTAA CGCGAATTTT AACAAAATAT TAACGTTTAC AATTTAAATA TTTGCTTATA |
| 3961 | CAATCTTCCT GTTTTTGGGG CTTTTCTGAT TATCAACCGG GGTACATATG ATTGACATGC |
| 4021 | TAGTTTTACG ATTACCGTTC ATCGATTCTC TTGTTTGCTC CAGACTCTCA GGCAATGACC |
| 4081 | TGATAGCCTT TGTAGAGACC TCTCAAAAAT AGCTACCCTC TCCGGCATGA ATTTATCAGC |
| 4141 | TAGAACGGTT GAATATCATA TTGATGGTGA TTTGACTGTC TCCGGCCTTT CTCACCCGTT |
| 4201 | TGAATCTTTA CCTACACATT ACTCAGGCAT TGCATTTAAA ATATATGAGG GTTCTAAAAA |
| 4261 | TTTTTATCCT TGCGTTGAAA TAAAGGCTTC TCCCGCAAAA GTATTACAGG GTCATAATGT |
| 4321 | TTTTGGTACA ACCGATTTAG CTTTATGCTC TGAGGCTTTA TTGCTTAATT TTGCTAATTC |
| 4381 | TTTGCCTTGC CTGTATGATT TATTGGATGT TGGAATCGCC TGATGCGGTA TTTTCTCCTT |
| 4441 | ACGCATCTGT GCGGTATTTC ACACCGCATA TGGTGCACTC TCAGTACAAT CTGCTCTGAT |
| 4501 | GCCGCATAGT TAAGCCAGCC CCGACACCCG CCAACACCCG CTGACGCGCC CTGACGGGCT |
| 4561 | TGTCTGCTCC CGGCATCCGC TTACAGACAA GCTGTGACCG TCTCCGGGAG CTGCATGTGT |
| 4621 | CAGAGGTTTT CACCGTCATC ACCGAAACGC GCGAGACGAA AGGGCCTCGT GATACGCCTA |
| 4681 | TTTTTATAGG TTAATGTCAT GATAATAATG GTTTCTTAGA CGTCAGGTGG CACTTTTCGG |
| 4741 | GGAAATGTGC GCGGAACCCC TATTTGTTTA TTTTTCTAAA TACATTCAAA TATGTATCCG |
| 4801 | CTCATGAGAC AATAACCCTG ATAAATGCTT CAATAATATT GAAAAAGGAA GAGTATGAGT |
| 4861 | ATTCAACATT TCCGTGTCGC CCTTATTCCC TTTTTTGCGG CATTTTGCCT TCCTGTTTTT |
| 4921 | GCTCACCCAG AAACGCTGGT GAAAGTAAAA GATGCTGAAG ATCAGTTGGG TGCACGAGTG |
| 4981 | GGTTACATCG AACTGGATCT CAACAGCGGT AAGATCCTTG AGAGTTTTCG CCCCGAAGAA |
| 5041 | CGTTTTCCAA TGATGAGCAC TTTTAAAGTT CTGCTATGTG GCGCGGTATT ATCCCGTATT |
| 5101 | GACGCCGGGC AAGAGCAACT CGGTCGCCGC ATACACTATT CTCAGAATGA CTTGGTTGAG |
| 5161 | TACTCACCAG TCACAGAAAA GCATCTTACG GATGGCATGA CAGTAAGAGA ATTATGCAGT |
| 5221 | GCTGCCATAA CCATGAGTGA TAACACTGCG GCCAACTTAC TTCTGACAAC GATCGGAGGA |
| 5281 | CCGAAGGAGC TAACCGCTTT TTTGCACAAC ATGGGGGATC ATGTAACTCG CCTTGATCGT |
| 5341 | TGGGAACCGG AGCTGAATGA AGCCATACCA AACGACGAGC GTGACACCAC GATGCCTGTA |
| 5401 | GCAATGGCAA CAACGTTGCG CAAACTATTA ACTGGCGAAC TACTTACTCT AGCTTCCCGG |
| 5461 | CAACAATTAA TAGACTGGAT GGAGGCGGAT AAAGTTGCAG GACCACTTCT GCGCTCGGCC |
| 5521 | CTTCCGGCTG GCTGGTTTAT TGCTGATAAA TCTGGAGCCG GTGAGCGTGG GTCTCGCGGT |
| 5581 | ATCATTGCAG CACTGGGGCC AGATGGTAAG CCCTCCCGTA TCGTAGTTAT CTACACGACG |
| 5641 | GGGAGTCAGG CAACTATGGA TGAACGAAAT AGACAGATCG CTGAGATAGG TGCCTCACTG |
| 5701 | ATTAAGCATT GGTAACTGTC AGACCAAGTT TACTCATATA TACTTTAGAT TGATTTAAAA |
| 5761 | CTTCATTTTT AATTTAAAAG GATCTAGGTG AAGATCCTTT TTGATAATCT CATGACCAAA |
| 5821 | ATCCCTTAAC GTGAGTTTTC GTTCCACTGA GCGTCAGACC CCGTAGAAAA GATCAAAGGA |
| 5881 | TCTTCTTGAG ATCCTTTTTT TCTGCGCGTA ATCTGCTGCT TGCAAACAAA AAAACCACCG |
| 5941 | CTACCAGCGG TGGTTTGTTT GCCGGATCAA GAGCTACCAA CTCTTTTTCC GAAGGTAACT |
| 6001 | GGCTTCAGCA GAGCGCAGAT ACCAAATACT GTCCTTCTAG TGTAGCCGTA GTTAGGCCAC |
| 6061 | CACTTCAAGA ACTCTGTAGC ACCGCCTACA TACCTCGCTC TGCTAATCCT GTTACCAGTG |
| 6121 | GCTGCTGCCA GTGGCGATAA GTCGTGTCTT ACCGGGTTGG ACTCAAGACG ATAGTTACCG |
| 6181 | GATAAGGCGC AGCGGTCGGG CTGAACGGGG GGTTCGTGCA CACAGCCCAG CTTGGAGCGA |
| 6241 | ACGACCTACA CCGAACTGAG ATACCTACAG CGTGAGCTAT GAGAAAGCGC CACGCTTCCC |
| 6301 | GAAGGGAGAA AGGCGGACAG GTATCCGGTA AGCGGCAGGG TCGGAACAGG AGAGCGCACG |
| 6361 | AGGGAGCTTC CAGGGGGAAA CGCCTGGTAT CTTTATAGTC CTGTCGGGTT TCGCCACCTC |
| 6421 | TGACTTGAGC GTCGATTTTT GTGATGCTCG TCAGGGGGGC GGAGCCTATG GAAAAACGCC |
| 6481 | AGCAACGCGG CCTTTTTACG GTTCCTGGCC TTTTGCTGGC CTTTTGCTCA CATGTTCTTT |
| 6541 | CCTGCGTTAT CCCCTGATTC TGTGGATAAC CGTATTACCG CCTTTGAGTG AGCTGATACC |
| 6601 | GCTCGCCGCA GCCGAACGAC CGAGCGCAGC GAGTCAGTGA GCGAGGAAGC GGAAGAGCGC |
| 6661 | CCAATACGCA AACCGCCTCT CCCCGCGCGT TGGCCGATTC ATTAATG |
| 1367_CCR5.MND.BAFF.2A.GFP | |
| (SEQ ID NO: 19) | |
| 1 | CAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGCGGCC TCGGCCTCTG CATAAATAAA AAAAATTAGT CAGCCATGAG CTTGGACGCG |
| 241 | TTTGGTGTGG TGGCGCCTGT AGTCCCCAGC CACTTGGAGG GGTGAGGTGA GAGGATTGCT |
| 301 | TGAGCCCGGG ATGGTCCAGG CTGCAGTGAG CCATGATCGT GCCACTGCAC TCCAGCCTGG |
| 361 | GCGACAGAGT GAGACCCTGT CTCACAACAA CAACAACAAC AACAAAAAGG CTGAGCTGCA |
| 421 | CCATGCTTGA CCCAGTTTCT TAAAATTGTT GTCAAAGCTT CATTCACTCC ATGGTGCTAT |
| 481 | AGAGCACAAG ATTTTATTTG GTGAGATGGT GCTTTCATGA ATTCCCCCAA CAGAGCCAAG |
| 541 | CTCTCCATCT AGTGGACAGG GAAGCTAGCA GCAAACCTTC CCTTCACTAC AAAACTTCAT |
| 601 | TGCTTGGCCA AAAAGAGAGT TAATTCAATG TAGACATCTA TGTAGGCAAT TAAAAACCTA |
| 661 | TTGATGTATA AAACAGTTTG CATTCATGGA GGGCAACTAA ATACATTCTA GGACTTTATA |
| 721 | AAAGATCACT TTTTATTTAT GCACAGGGTG GAACAAGATG GATTATCAAG TGTCAAGTCC |
| 781 | AATCTATGAC ATCAATTATT ATACATCGGA GCCCTGCCAA AAAATCAATG TGAAGCAAAT |
| 841 | CGCAGCCCGC CTCCTGCCTC CGCTCTACTC ACTGGTGTTC ATCTTTGGTT TTGTGGGCAA |
| 901 | CATGCTGGTC ATCCTCATCC TGATAAACTG CAAAAGGCTG AAGAGCATGA CTGACATCTA |
| 961 | CCTGCTCAAC CTGGCCATCT CTGACCTGTT TTTCCTTCTT ACTGTCCCCT TCTGGGCTCA |
| 1021 | CTATGCTGCC GCCCAGTGGG ACTTTGGAAA TACAATGTGT CAACGAACAG AGAAACAGGA |
| 1081 | GAATATGGGC CAAACAGGAT ATCTGTGGTA AGCAGTTCCT GCCCCGGCTC AGGGCCAAGA |
| 1141 | ACAGTTGGAA CAGCAGAATA TGGGCCAAAC AGGATATCTG TGGTAAGCAG TTCCTGCCCC |
| 1201 | GGCTCAGGGC CAAGAACAGA TGGTCCCCAG ATGCGGTCCC GCCCTCAGCA GTTTCTAGAG |
| 1261 | AACCATCAGA TGTTTCCAGG GTGCCCCAAG GACCTGAAAT GACCCTGTGC CTTATTTGAA |
| 1321 | CTAACCAATC AGTTCGCTTC TCGCTTCTGT TCGCGCGCTT CTGCTCCCCG AGCTCTATAT |
| 1381 | AAGCAGAGCT CGTTTAGTGA ACCGTCAGAT CGCCTGGAGA CGCCATCCAC GCTGTTTTGA |
| 1441 | CTTCCATAGA AGGATCTCGA GGCCACCATG TACCGGATGC AGCTGCTGAG CTGCATCGCA |
| 1501 | CTGAGCCTGG CACTGGTGAC CAACAGCGCA GTGCAGGGAC CAGAGGAGAC CGTGACCCAG |
| 1561 | GACTGCCTGC AGCTGATCGC AGACAGCGAG ACCCCCACCA TCCAGAAGGG CAGCTACACC |
| 1621 | TTCGTGCCCT GGCTGCTGAG CTTCAAGCGG GGCAGCGCCC TGGAGGAGAA GGAGAACAAG |
| 1681 | ATTCTGGTGA AGGAGACCGG CTACTTCTTC ATCTACGGCC AGGTGCTGTA CACCGATAAG |
| 1741 | ACCTACGCCA TGGGCCACCT GATCCAGCGG AAGAAGGTGC ACGTGTTCGG CGACGAGCTG |
| 1801 | AGCCTGGTGA CCCTGTTCCG GTGCATCCAG AACATGCCCG AGACCCTGCC CAACAACAGC |
| 1861 | TGCTACAGCG CAGGAATCGC AAAGCTGGAG GAGGGCGACG AGCTGCAGCT GGCAATCCCC |
| 1921 | CGGGAGAACG CACAGATCAG CCTGGACGGC GACGTGACCT TCTTCGGCGC CCTGAAGCTC |
| 1981 | CTGGGATCCG GTGAGGGCAG AGGAAGTCTT CTAACATGCG GTGACGTGGA GGAGAATCCG |
| 2041 | GGCCCCATGG TGAGCAAGGG CGAGGAGCTG TTCACCGGGG TGGTGCCCAT CCTGGTCGAG |
| 2101 | CTGGACGGCG ACGTAAACGG CCACAAGTTC AGCGTGTCCG GCGAGGGCGA GGGCGATGCC |
| 2161 | ACCTACGGCA AGCTGACCCT GAAGTTCATC TGCACCACCG GCAAGCTGCC CGTGCCCTGG |
| 2221 | CCCACCCTCG TGACCACCCT GACCTACGGC GTGCAGTGCT TCAGCCGCTA CCCCGACCAC |
| 2281 | ATGAAGCAGC ACGACTTCTT CAAGTCCGCC ATGCCCGAAG GCTACGTCCA GGAGCGCACC |
| 2341 | ATCTTCTTCA AGGACGACGG CAACTACAAG ACCCGCGCCG AGGTGAAGTT CGAGGGCGAC |
| 2401 | ACCCTGGTGA ACCGCATCGA GCTGAAGGGC ATCGACTTCA AGGAGGACGG CAACATCCTG |
| 2461 | GGGCACAAGC TGGAGTACAA CTACAACAGC CACAACGTCT ATATCATGGC CGACAAGCAG |
| 2521 | AAGAACGGCA TCAAGGTGAA CTTCAAGATC CGCCACAACA TCGAGGACGG CAGCGTGCAG |
| 2581 | CTCGCCGACC ACTACCAGCA GAACACCCCC ATCGGCGACG GCCCCGTGCT GCTGCCCGAC |
| 2641 | AACCACTACC TGAGCACCCA GTCCGCCCTG AGCAAAGACC CCAACGAGAA GCGCGATCAC |
| 2701 | ATGGTCCTGC TGGAGTTCGT GACCGCCGCC GGGATCACTC TCGGCATGGA CGAGCTGTAC |
| 2761 | AAGTGAATCT AGAGTCGACT GCTTTATTTG TGAAATTTGT GATGCTATTG CTTTATTTGT |
| 2821 | AACCATTATA AGCTGCAATA AACAAGTTAA CAACAACAAT TGCATTCATT TTATGTTTCA |
| 2881 | GGTTCAGGGG GAGGTGTGGG AGGTTTTTTA AACTCTATTT TATAGGCTTC TTCTCTGGAA |
| 2941 | TCTTCTTCAT CATCCTCCTG ACAATCGATA GGTACCTGGC TGTCGTCCAT GCTGTGTTTG |
| 3001 | CTTTAAAAGC CAGGACGGTC ACCTTTGGGG TGGTGACAAG TGTGATCACT TGGGTGGTGG |
| 3061 | CTGTGTTTGC GTCTCTCCCA GGAATCATCT TTACCAGATC TCAAAAAGAA GGTCTTCATT |
| 3121 | ACACCTGCAG CTCTCATTTT CCATACAGTC AGTATCAATT CTGGAAGAAT TTCCAGACAT |
| 3181 | TAAAGATAGT CATCTTGGGG CTGGTCCTGC CGCTGCTTGT CATGGTCATC TGCTACTCGG |
| 3241 | GAATCCTAAA AACTCTGCTT CGGTGTCGAA ATGAGAAGAA GAGGCACAGG GCTGTGAGGC |
| 3301 | TTATCTTCAC CATCATGATT GTTTATTTTC TCTTCTGGGC TCCCTACAAC ATTGTCCTTC |
| 3361 | TCCTGAACAC CTTCCAGGAA TTCTTTGGCC TGAATAATTG CAGTAGCTCT AACAGGTTGG |
| 3421 | ACCAAGCTAT GCAGGTGACA GAGACTCTTG GGATGACGCA CTGCTGCATC AACCCCATCA |
| 3481 | TCTATGCCTT TGTCGGGGAG AAGTTCAGAA ACTACCTCTT AGTCTTCTTC CAAAAGCACA |
| 3541 | TTGCCAAACG CTTCTGCAAA TGCTGTTCTA TTTTCCAGCA AGAGGCTCCC GAGCGAGCAA |
| 3601 | GCTCAGTTTA CACCCGATCC ACTGGGGAGC AGGAAATATC TGTGGGCTTG TGACACGGAC |
| 3661 | TCAAGTGGGC TGGTGACCCA GTCAGAGTTG TGCACATGGC TTAGTTTTCA TACACACCGC |
| 3721 | GGTCTAGAGC ATGGCTACGT AGATAAGTAG CATGGCGGGT TAATCATTAA CTACAAGGAA |
| 3781 | CCCCTAGTGA TGGAGTTGGC CACTCCCTCT CTGCGCGCTC GCTCGCTCAC TGAGGCCGGG |
| 3841 | CGACCAAAGG TCGCCCGACG CCCGGGCTTT GCCCGGGCGG CCTCAGTGAG CGAGCGAGCG |
| 3901 | CGCCAGCTGG CGTAATAGCG AAGAGGCCCG CACCGATCGC CCTTCCCAAC AGTTGCGCAG |
| 3961 | CCTGAATGGC GAATGGCGAT TCCGTTGCAA TGGCTGGCGG TAATATTGTT CTGGATATTA |
| 4021 | CCAGCAAGGC CGATAGTTTG AGTTCTTCTA CTCAGGCAAG TGATGTTATT ACTAATCAAA |
| 4081 | GAAGTATTGC GACAACGGTT AATTTGCGTG ATGGACAGAC TCTTTTACTC GGTGGCCTCA |
| 4141 | CTGATTATAA AAACACTTCT CAGGATTCTG GCGTACCGTT CCTGTCTAAA ATCCCTTTAA |
| 4201 | TCGGCCTCCT GTTTAGCTCC CGCTCTGATT CTAACGAGGA AAGCACGTTA TACGTGCTCG |
| 4261 | TCAAAGCAAC CATAGTACGC GCCCTGTAGC GGCGCATTAA GCGCGGCGGG TGTGGTGGTT |
| 4321 | ACGCGCAGCG TGACCGCTAC ACTTGCCAGC GCCCTAGCGC CCGCTCCTTT CGCTTTCTTC |
| 4381 | CCTTCCTTTC TCGCCACGTT CGCCGGCTTT CCCCGTCAAG CTCTAAATCG GGGGCTCCCT |
| 4441 | TTAGGGTTCC GATTTAGTGC TTTACGGCAC CTCGACCCCA AAAAACTTGA TTAGGGTGAT |
| 4501 | GGTTCACGTA GTGGGCCATC GCCCTGATAG ACGGTTTTTC GCCCTTTGAC GTTGGAGTCC |
| 4561 | ACGTTCTTTA ATAGTGGACT CTTGTTCCAA ACTGGAACAA CACTCAACCC TATCTCGGTC |
| 4621 | TATTCTTTTG ATTTATAAGG GATTTTGCCG ATTTCGGCCT ATTGGTTAAA AAATGAGCTG |
| 4681 | ATTTAACAAA AATTTAACGC GAATTTTAAC AAAATATTAA CGTTTACAAT TTAAATATTT |
| 4741 | GCTTATACAA TCTTCCTGTT TTTGGGGCTT TTCTGATTAT CAACCGGGGT ACATATGATT |
| 4801 | GACATGCTAG TTTTACGATT ACCGTTCATC GATTCTCTTG TTTGCTCCAG ACTCTCAGGC |
| 4861 | AATGACCTGA TAGCCTTTGT AGAGACCTCT CAAAAATAGC TACCCTCTCC GGCATGAATT |
| 4921 | TATCAGCTAG AACGGTTGAA TATCATATTG ATGGTGATTT GACTGTCTCC GGCCTTTCTC |
| 4981 | ACCCGTTTGA ATCTTTACCT ACACATTACT CAGGCATTGC ATTTAAAATA TATGAGGGTT |
| 5041 | CTAAAAATTT TTATCCTTGC GTTGAAATAA AGGCTTCTCC CGCAAAAGTA TTACAGGGTC |
| 5101 | ATAATGTTTT TGGTACAACC GATTTAGCTT TATGCTCTGA GGCTTTATTG CTTAATTTTG |
| 5161 | CTAATTCTTT GCCTTGCCTG TATGATTTAT TGGATGTTGG AATCGCCTGA TGCGGTATTT |
| 5221 | TCTCCTTACG CATCTGTGCG GTATTTCACA CCGCATATGG TGCACTCTCA GTACAATCTG |
| 5281 | CTCTGATGCC GCATAGTTAA GCCAGCCCCG ACACCCGCCA ACACCCGCTG ACGCGCCCTG |
| 5341 | ACGGGCTTGT CTGCTCCCGG CATCCGCTTA CAGACAAGCT GTGACCGTCT CCGGGAGCTG |
| 5401 | CATGTGTCAG AGGTTTTCAC CGTCATCACC GAAACGCGCG AGACGAAAGG GCCTCGTGAT |
| 5461 | ACGCCTATTT TTATAGGTTA ATGTCATGAT AATAATGGTT TCTTAGACGT CAGGTGGCAC |
| 5521 | TTTTCGGGGA AATGTGCGCG GAACCCCTAT TTGTTTATTT TTCTAAATAC ATTCAAATAT |
| 5581 | GTATCCGCTC ATGAGACAAT AACCCTGATA AATGCTTCAA TAATATTGAA AAAGGAAGAG |
| 5641 | TATGAGTATT CAACATTTCC GTGTCGCCCT TATTCCCTTT TTTGCGGCAT TTTGCCTTCC |
| 5701 | TGTTTTTGCT CACCCAGAAA CGCTGGTGAA AGTAAAAGAT GCTGAAGATC AGTTGGGTGC |
| 5761 | ACGAGTGGGT TACATCGAAC TGGATCTCAA CAGCGGTAAG ATCCTTGAGA GTTTTCGCCC |
| 5821 | CGAAGAACGT TTTCCAATGA TGAGCACTTT TAAAGTTCTG CTATGTGGCG CGGTATTATC |
| 5881 | CCGTATTGAC GCCGGGCAAG AGCAACTCGG TCGCCGCATA CACTATTCTC AGAATGACTT |
| 5941 | GGTTGAGTAC TCACCAGTCA CAGAAAAGCA TCTTACGGAT GGCATGACAG TAAGAGAATT |
| 6001 | ATGCAGTGCT GCCATAACCA TGAGTGATAA CACTGCGGCC AACTTACTTC TGACAACGAT |
| 6061 | CGGAGGACCG AAGGAGCTAA CCGCTTTTTT GCACAACATG GGGGATCATG TAACTCGCCT |
| 6121 | TGATCGTTGG GAACCGGAGC TGAATGAAGC CATACCAAAC GACGAGCGTG ACACCACGAT |
| 6181 | GCCTGTAGCA ATGGCAACAA CGTTGCGCAA ACTATTAACT GGCGAACTAC TTACTCTAGC |
| 6241 | TTCCCGGCAA CAATTAATAG ACTGGATGGA GGCGGATAAA GTTGCAGGAC CACTTCTGCG |
| 6301 | CTCGGCCCTT CCGGCTGGCT GGTTTATTGC TGATAAATCT GGAGCCGGTG AGCGTGGGTC |
| 6361 | TCGCGGTATC ATTGCAGCAC TGGGGCCAGA TGGTAAGCCC TCCCGTATCG TAGTTATCTA |
| 6421 | CACGACGGGG AGTCAGGCAA CTATGGATGA ACGAAATAGA CAGATCGCTG AGATAGGTGC |
| 6481 | CTCACTGATT AAGCATTGGT AACTGTCAGA CCAAGTTTAC TCATATATAC TTTAGATTGA |
| 6541 | TTTAAAACTT CATTTTTAAT TTAAAAGGAT CTAGGTGAAG ATCCTTTTTG ATAATCTCAT |
| 6601 | GACCAAAATC CCTTAACGTG AGTTTTCGTT CCACTGAGCG TCAGACCCCG TAGAAAAGAT |
| 6661 | CAAAGGATCT TCTTGAGATC CTTTTTTTCT GCGCGTAATC TGCTGCTTGC AAACAAAAAA |
| 6721 | ACCACCGCTA CCAGCGGTGG TTTGTTTGCC GGATCAAGAG CTACCAACTC TTTTTCCGAA |
| 6781 | GGTAACTGGC TTCAGCAGAG CGCAGATACC AAATACTGTC CTTCTAGTGT AGCCGTAGTT |
| 6841 | AGGCCACCAC TTCAAGAACT CTGTAGCACC GCCTACATAC CTCGCTCTGC TAATCCTGTT |
| 6901 | ACCAGTGGCT GCTGCCAGTG GCGATAAGTC GTGTCTTACC GGGTTGGACT CAAGACGATA |
| 6961 | GTTACCGGAT AAGGCGCAGC GGTCGGGCTG AACGGGGGGT TCGTGCACAC AGCCCAGCTT |
| 7021 | GGAGCGAACG ACCTACACCG AACTGAGATA CCTACAGCGT GAGCTATGAG AAAGCGCCAC |
| 7081 | GCTTCCCGAA GGGAGAAAGG CGGACAGGTA TCCGGTAAGC GGCAGGGTCG GAACAGGAGA |
| 7141 | GCGCACGAGG GAGCTTCCAG GGGGAAACGC CTGGTATCTT TATAGTCCTG TCGGGTTTCG |
| 7201 | CCACCTCTGA CTTGAGCGTC GATTTTTGTG ATGCTCGTCA GGGGGGCGGA GCCTATGGAA |
| 7261 | AAACGCCAGC AACGCGGCCT TTTTACGGTT CCTGGCCTTT TGCTGGCCTT TTGCTCACAT |
| 7321 | GTTCTTTCCT GCGTTATCCC CTGATTCTGT GGATAACCGT ATTACCGCCT TTGAGTGAGC |
| 7381 | TGATACCGCT CGCCGCAGCC GAACGACCGA GCGCAGCGAG TCAGTGAGCG AGGAAGCGGA |
| 7441 | AGAGCGCCCA ATACGCAAAC CGCCTCTCCC CGCGCGTTGG CCGATTCATT AATG |
| SEQ ID NO: 51 | |
| 1079_pscAAV-MND.GFP | |
| 1079_pscAAV-MND.GFP | |
| (SEQ ID NO: 51) | |
| ORIGIN | |
| 1 | aagcttcccg gggggatctg ggccactccc tctctgcgcg ctcgctcgct cactgaggcc |
| 61 | gggcgaccaa aggtcgcccg acgcccgggc tttgcccggg cggcctcagt gagcgagcga |
| 121 | gcgcgcagag agggagtggc caactccatc actaggggtt cctggagggg tggagtcgtg |
| 181 | acctagggaa cagagaaaca ggagaatatg ggccaaacag gatatctgtg gtaagcagtt |
| 241 | cctgccccgg ctcagggcca agaacagttg gaacagcaga atatgggcca aacaggatat |
| 301 | ctgtggtaag cagttcctgc cccggctcag ggccaagaac agatggtccc cagatgcggt |
| 361 | cccgccctca gcagtttcta gagaaccatc agatgtttcc agggtgcccc aaggacctga |
| 421 | aatgaccctg tgccttattt gaactaacca atcagttcgc ttctcgcttc tgttcgcgcg |
| 481 | cttctgctcc ccgagctcta tataagcaga gctcgtttag tgaaccgtca gatcgcctgg |
| 541 | agacgccatc cacgctgttt tgacttccat agaaggatcc tcgaggccac catggtgagc |
| 601 | aagggcgagg agctgttcac cggggtggtg cccatcctgg tcgagctgga cggcgacgta |
| 661 | aacggccaca agttcagcgt gtccggcgag ggcgagggcg atgccaccta cggcaagctg |
| 721 | accctgaagt tcatctgcac caccggcaag ctgcccgtgc cctggcccac cctcgtgacc |
| 781 | accctgacct acggcgtgca gtgcttcagc cgctaccccg accacatgaa gcagcacgac |
| 841 | ttcttcaagt ccgccatgcc cgaaggctac gtccaggagc gcaccatctt cttcaaggac |
| 901 | gacggcaact acaagacccg cgccgaggtg aagttcgagg gcgacaccct ggtgaaccgc |
| 961 | atcgagctga agggcatcga cttcaaggag gacggcaaca tcctggggca caagctggag |
| 1021 | tacaactaca acagccacaa cgtctatatc atggccgaca agcagaagaa cggcatcaag |
| 1081 | gtgaacttca agatccgcca caacatcgag gacggcagcg tgcagctcgc cgaccactac |
| 1141 | cagcagaaca cccccatcgg cgacggcccc gtgctgctgc ccgacaacca ctacctgagc |
| 1201 | acccagtccg ccctgagcaa agaccccaac gagaagcgcg atcacatggt cctgctggag |
| 1261 | ttcgtgaccg ccgccgggat cactctcggc atggacgagc tgtacaagta agcggccgca |
| 1321 | attcacccca ccagtgcagg ctgcctatca gaaagtggtg gctggtgtgg ctaatgccct |
| 1381 | ggcccacaag tatcactaag ctcgctttct tgctgtccaa tttctattaa aggttccttt |
| 1441 | gttccctaag tccaactact aaactggggg atattatgaa gggccttgag catctggatt |
| 1501 | ctgcctaata aaaaacattt attttcattg caatgatgta tttaaattat ttctgaatat |
| 1561 | tttactaaaa agggaatgtg ggaggtcagt gcatttaaaa cataaagaaa tgaagagcta |
| 1621 | gttcaaacct tgggaaaata cactatatct taaactccat gaaagaaggt gaggctgcaa |
| 1681 | acagctaatg cacattggca acagcccctg atgcctatgc cttattcatc cctcagaaaa |
| 1741 | ggattcaagt agaggcttga tttggaggtt aaagttttgc tatgctgtat tttacattac |
| 1801 | ttattgtttt agctgtcctc atgaatgtct tttcactacc catttgctta tcctgcatct |
| 1861 | ctcagccttg actccactca gttctcttgc ttagagatac cacctttccc ctgaagtgtt |
| 1921 | ccttccatgt tttacggcga gatggtttct cctcgcctgg ccactcagcc ttagttgtct |
| 1981 | ctgttgtctt atagaggtct acttgaagaa ggaaaaacag ggggcatggt ttgactgtcc |
| 2041 | tgtgagccct tcttccctgc ctcccccact cacagtgaca ctagtccact ccctctctgc |
| 2101 | gcgctcgctc gctcactgag gccgggcgac caaaggtcgc ccgacgcccg ggctttgccc |
| 2161 | gggcggcctc agtgagcgag cgagcgcgca gagagggaca gatccgggcc cgcatgcgtc |
| 2221 | gacaattcac tggccgtcgt tttacaacgt cgtgactggg aaaaccctgg cgttacccaa |
| 2281 | cttaatcgcc ttgcagcaca tccccctttc gccagctggc gtaatagcga agaggcccgc |
| 2341 | accgatcgcc cttcccaaca gttgcgcagc ctgaatggcg aatggcgcct gatgcggtat |
| 2401 | tttctcctta cgcatctgtg cggtatttca caccgcatat ggtgcactct cagtacaatc |
| 2461 | tgctctgatg ccgcatagtt aagccagccc cgacacccgc caacacccgc tgacgcgccc |
| 2521 | tgacgggctt gtctgctccc ggcatccgct tacagacaag ctgtgaccgt ctccgggagc |
| 2581 | tgcatgtgtc agaggttttc accgtcatca ccgaaacgcg cgagacgaaa gggcctcgtg |
| 2641 | atacgcctat ttttataggt taatgtcatg ataataatgg tttcttagac gtcaggtggc |
| 2701 | acttttcggg gaaatgtgcg cggaacccct atttgtttat ttttctaaat acattcaaat |
| 2761 | atgtatccgc tcatgagaca ataaccctga taaatgcttc aataatattg aaaaaggaag |
| 2821 | agtatgagta ttcaacattt ccgtgtcgcc cttattccct tttttgcggc attttgcctt |
| 2881 | cctgtttttg ctcacccaga aacgctggtg aaagtaaaag atgctgaaga tcagttgggt |
| 2941 | gcacgagtgg gttacatcga actggatctc aacagcggta agatccttga gagttttcgc |
| 3001 | cccgaagaac gttttccaat gatgagcact tttaaagttc tgctatgtgg cgcggtatta |
| 3061 | tcccgtattg acgccgggca agagcaactc ggtcgccgca tacactattc tcagaatgac |
| 3121 | ttggttgagt actcaccagt cacagaaaag catcttacgg atggcatgac agtaagagaa |
| 3181 | ttatgcagtg ctgccataac catgagtgat aacactgcgg ccaacttact tctgacaacg |
| 3241 | atcggaggac cgaaggagct aaccgctttt ttgcacaaca tgggggatca tgtaactcgc |
| 3301 | cttgatcgtt gggaaccgga gctgaatgaa gccataccaa acgacgagcg tgacaccacg |
| 3361 | atgcctgtag caatggcaac aacgttgcgc aaactattaa ctggcgaact acttactcta |
| 3421 | gcttcccggc aacaattaat agactggatg gaggcggata aagttgcagg accacttctg |
| 3481 | cgctcggccc ttccggctgg ctggtttatt gctgataaat ctggagccgg tgagcgtggg |
| 3541 | tctcgcggta tcattgcagc actggggcca gatggtaagc cctcccgtat cgtagttatc |
| 3601 | tacacgacgg ggagtcaggc aactatggat gaacgaaata gacagatcgc tgagataggt |
| 3661 | gcctcactga ttaagcattg gtaactgtca gaccaagttt actcatatat actttagatt |
| 3721 | gatttaaaac ttcattttta atttaaaagg atctaggtga agatcctttt tgataatctc |
| 3781 | atgaccaaaa tcccttaacg tgagttttcg ttccactgag cgtcagaccc cgtagaaaag |
| 3841 | atcaaaggat cttcttgaga tccttttttt ctgcgcgtaa tctgctgctt gcaaacaaaa |
| 3901 | aaaccaccgc taccagcggt ggtttgtttg ccggatcaag agctaccaac tctttttccg |
| 3961 | aaggtaactg gcttcagcag agcgcagata ccaaatactg ttcttctagt gtagccgtag |
| 4021 | ttaggccacc acttcaagaa ctctgtagca ccgcctacat acctcgctct gctaatcctg |
| 4081 | ttaccagtgg ctgctgccag tggcgataag tcgtgtctta ccgggttgga ctcaagacga |
| 4141 | tagttaccgg ataaggcgca gcggtcgggc tgaacggggg gttcgtgcac acagcccagc |
| 4201 | ttggagcgaa cgacctacac cgaactgaga tacctacagc gtgagctatg agaaagcgcc |
| 4261 | acgcttcccg aagggagaaa ggcggacagg tatccggtaa gcggcagggt cggaacagga |
| 4321 | gagcgcacga gggagcttcc agggggaaac gcctggtatc tttatagtcc tgtcgggttt |
| 4381 | cgccacctct gacttgagcg tcgatttttg tgatgctcgt caggggggcg gagcctatgg |
| 4441 | aaaaacgcca gcaacgcggc ctttttacgg ttcctggcct tttgctggcc ttttgctcac |
| 4501 | atgttctttc ctgcgttatc ccctgattct gtggataacc gtattaccgc ctttgagtga |
| 4561 | gctgataccg ctcgccgcag ccgaacgacc gagcgcagcg agtcagtgag cgaggaagcg |
| 4621 | gaagagcgcc caatacgcaa accgcctctc cccgcgcgtt ggccgattca ttaatgcagc |
| 4681 | tggcacgaca ggtttcccga ctggaaagcg ggcagtgagc gcaacgcaat taatgtgagt |
| 4741 | tagctcactc attaggcacc ccaggcttta cactttatgc ttccggctcg tatgttgtgt |
| 4801 | ggaattgtga gcggataaca atttcacaca ggaaacagct atgaccatga ttacgccaag |
| 4861 | ctctcgagat ctaga |
| 1347_pscAAV.Blimp.0.4 kb.MND.GFP | |
| (SEQ ID NO: 52) | |
| 1 | aagcttcccg gggggatctg ggccactccc tctctgcgcg ctcgctcgct cactgaggcc |
| 61 | gggcgaccaa aggtcgcccg acgcccgggc tttgcccggg cggcctcagt gagcgagcga |
| 121 | gcgcgcagag agggagtggc caactccatc actaggggtt cctggagggg tggagtcgtg |
| 181 | acctaggacg cgtgccagct gttactcagg ttttctcaag aaggaggagc aactttggca |
| 241 | gttttgcttc agttctctct agccctctgt gtaatcgccc ctttttcttt atttcagcac |
| 301 | aaacacagag cagtctaaag caaccgagca ctgagaaaaa tgaactctgc ccaaagaatg |
| 361 | tcccaaagag agagtacagc gtgaaagaaa tcctaaaatt ggactccaac ccctccaaag |
| 421 | gaaaggacct ctaccgttct aacatttcac ccctcacatc agaaaaggac ctcgatgact |
| 481 | ttagaagacg tgggagcccc gaaatgccct tctaccctcg ggtcgtttac cccatccggg |
| 541 | cccctctgcc agaagacttt ttgaaagctt ccctggccta cgggatcgag agagaacaga |
| 601 | gaaacaggag aatatgggcc aaacaggata tctgtggtaa gcagttcctg ccccggctca |
| 661 | gggccaagaa cagttggaac agcagaatat gggccaaaca ggatatctgt ggtaagcagt |
| 721 | tcctgccccg gctcagggcc aagaacagat ggtccccaga tgcggtcccg ccctcagcag |
| 781 | tttctagaga accatcagat gtttccaggg tgccccaagg acctgaaatg accctgtgcc |
| 841 | ttatttgaac taaccaatca gttcgcttct cgcttctgtt cgcgcgcttc tgctccccga |
| 901 | gctctatata agcagagctc gtttagtgaa ccgtcagatc gcctggagac gccatccacg |
| 961 | ctgttttgac ttccatagaa ggatctcgag gccaccatgg tgagcaaggg cgaggagctg |
| 1021 | ttcaccgggg tggtgcccat cctggtcgag ctggacggcg acgtaaacgg ccacaagttc |
| 1081 | agcgtgtccg gcgagggcga gggcgatgcc acctacggca agctgaccct gaagttcatc |
| 1141 | tgcaccaccg gcaagctgcc cgtgccctgg cccaccctcg tgaccaccct gacctacggc |
| 1201 | gtgcagtgct tcagccgcta ccccgaccac atgaagcagc acgacttctt caagtccgcc |
| 1261 | atgcccgaag gctacgtcca ggagcgcacc atcttcttca aggacgacgg caactacaag |
| 1321 | acccgcgccg aggtgaagtt cgagggcgac accctggtga accgcatcga gctgaagggc |
| 1381 | atcgacttca aggaggacgg caacatcctg gggcacaagc tggagtacaa ctacaacagc |
| 1441 | cacaacgtct atatcatggc cgacaagcag aagaacggca tcaaggtgaa cttcaagatc |
| 1501 | cgccacaaca tcgaggacgg cagcgtgcag ctcgccgacc actaccagca gaacaccccc |
| 1561 | atcggcgacg gccccgtgct gctgcccgac aaccactacc tgagcaccca gtccgccctg |
| 1621 | agcaaagacc ccaacgagaa gcgcgatcac atggtcctgc tggagttcgt gaccgccgcc |
| 1681 | gggatcactc tcggcatgga cgagctgtac aagtaaacta gtgtcgactg ctttatttgt |
| 1741 | gaaatttgtg atgctattgc tttatttgta accattataa gctgcaataa acaagttaac |
| 1801 | aacaacaatt gcattcattt tatgtttcag gttcaggggg aggtgtggga ggttttttaa |
| 1861 | aacgtacatc actcgctccc ccattccatc ctccaccact ccaagcccct ctgcaagaag |
| 1921 | cagccccgac caaagcctca agagctccag ccctcacagc agccctggga atacggtgtc |
| 1981 | ccctgtgggc cccggctctc aagagcaccg ggactcctac gcttacttga acgcgtccta |
| 2041 | cggcacggaa ggtttgggct cctaccctgg ctacgcaccc ctgccccacc tcccgccagc |
| 2101 | tttcatcccc tcgtacaacg ctcactaccc caagttcctc ttgcccccct acggcatgaa |
| 2161 | ttgtaatggc ctgagcgctg tgagcagcat gaatggcatc aacaactttg gcctcttccc |
| 2221 | gaggctgtgc cctgtctaca gcaatctcct cggtgggggc actagtccac tccctctctg |
| 2281 | cgcgctcgct cgctcactga ggccgggcga ccaaaggtcg cccgacgccc gggctttgcc |
| 2341 | cgggcggcct cagtgagcga gcgagcgcgc agagagggac agatccgggc ccgcatgcgt |
| 2401 | cgacaattca ctggccgtcg ttttacaacg tcgtgactgg gaaaaccctg gcgttaccca |
| 2461 | acttaatcgc cttgcagcac atcccccttt cgccagctgg cgtaatagcg aagaggcccg |
| 2521 | caccgatcgc ccttcccaac agttgcgcag cctgaatggc gaatggcgcc tgatgcggta |
| 2581 | ttttctcctt acgcatctgt gcggtatttc acaccgcata tggtgcactc tcagtacaat |
| 2641 | ctgctctgat gccgcatagt taagccagcc ccgacacccg ccaacacccg ctgacgcgcc |
| 2701 | ctgacgggct tgtctgctcc cggcatccgc ttacagacaa gctgtgaccg tctccgggag |
| 2761 | ctgcatgtgt cagaggtttt caccgtcatc accgaaacgc gcgagacgaa agggcctcgt |
| 2821 | gatacgccta tttttatagg ttaatgtcat gataataatg gtttcttaga cgtcaggtgg |
| 2881 | cacttttcgg ggaaatgtgc gcggaacccc tatttgttta tttttctaaa tacattcaaa |
| 2941 | tatgtatccg ctcatgagac aataaccctg ataaatgctt caataatatt gaaaaaggaa |
| 3001 | gagtatgagt attcaacatt tccgtgtcgc ccttattccc ttttttgcgg cattttgcct |
| 3061 | tcctgttttt gctcacccag aaacgctggt gaaagtaaaa gatgctgaag atcagttggg |
| 3121 | tgcacgagtg ggttacatcg aactggatct caacagcggt aagatccttg agagttttcg |
| 3181 | ccccgaagaa cgttttccaa tgatgagcac ttttaaagtt ctgctatgtg gcgcggtatt |
| 3241 | atcccgtatt gacgccgggc aagagcaact cggtcgccgc atacactatt ctcagaatga |
| 3301 | cttggttgag tactcaccag tcacagaaaa gcatcttacg gatggcatga cagtaagaga |
| 3361 | attatgcagt gctgccataa ccatgagtga taacactgcg gccaacttac ttctgacaac |
| 3421 | gatcggagga ccgaaggagc taaccgcttt tttgcacaac atgggggatc atgtaactcg |
| 3481 | ccttgatcgt tgggaaccgg agctgaatga agccatacca aacgacgagc gtgacaccac |
| 3541 | gatgcctgta gcaatggcaa caacgttgcg caaactatta actggcgaac tacttactct |
| 3601 | agcttcccgg caacaattaa tagactggat ggaggcggat aaagttgcag gaccacttct |
| 3661 | gcgctcggcc cttccggctg gctggtttat tgctgataaa tctggagccg gtgagcgtgg |
| 3721 | gtctcgcggt atcattgcag cactggggcc agatggtaag ccctcccgta tcgtagttat |
| 3781 | ctacacgacg gggagtcagg caactatgga tgaacgaaat agacagatcg ctgagatagg |
| 3841 | tgcctcactg attaagcatt ggtaactgtc agaccaagtt tactcatata tactttagat |
| 3901 | tgatttaaaa cttcattttt aatttaaaag gatctaggtg aagatccttt ttgataatct |
| 3961 | catgaccaaa atcccttaac gtgagttttc gttccactga gcgtcagacc ccgtagaaaa |
| 4021 | gatcaaagga tcttcttgag atcctttttt tctgcgcgta atctgctgct tgcaaacaaa |
| 4081 | aaaaccaccg ctaccagcgg tggtttgttt gccggatcaa gagctaccaa ctctttttcc |
| 4141 | gaaggtaact ggcttcagca gagcgcagat accaaatact gttcttctag tgtagccgta |
| 4201 | gttaggccac cacttcaaga actctgtagc accgcctaca tacctcgctc tgctaatcct |
| 4261 | gttaccagtg gctgctgcca gtggcgataa gtcgtgtctt accgggttgg actcaagacg |
| 4321 | atagttaccg gataaggcgc agcggtcggg ctgaacgggg ggttcgtgca cacagcccag |
| 4381 | cttggagcga acgacctaca ccgaactgag atacctacag cgtgagctat gagaaagcgc |
| 4441 | cacgcttccc gaagggagaa aggcggacag gtatccggta agcggcaggg tcggaacagg |
| 4501 | agagcgcacg agggagcttc cagggggaaa cgcctggtat ctttatagtc ctgtcgggtt |
| 4561 | tcgccacctc tgacttgagc gtcgattttt gtgatgctcg tcaggggggc ggagcctatg |
| 4621 | gaaaaacgcc agcaacgcgg cctttttacg gttcctggcc ttttgctggc cttttgctca |
| 4681 | catgttcttt cctgcgttat cccctgattc tgtggataac cgtattaccg cctttgagtg |
| 4741 | agctgatacc gctcgccgca gccgaacgac cgagcgcagc gagtcagtga gcgaggaagc |
| 4801 | ggaagagcgc ccaatacgca aaccgcctct ccccgcgcgt tggccgattc attaatgcag |
| 4861 | ctggcacgac aggtttcccg actggaaagc gggcagtgag cgcaacgcaa ttaatgtgag |
| 4921 | ttagctcact cattaggcac cccaggcttt acactttatg cttccggctc gtatgttgtg |
| 4981 | tggaattgtg agcggataac aatttcacac aggaaacagc tatgaccatg attacgccaa |
| 5041 | gctctcgaga tctaga |
| 1348_pAAV.Blimp.0.4 kb.MND.GFP.pA | |
| (SEQ ID NO: 53) | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac acgcgtgcca |
| 181 | gctgttactc aggttttctc aagaaggagg agcaactttg gcagttttgc ttcagttctc |
| 241 | tctagccctc tgtgtaatcg cccctttttc tttatttcag cacaaacaca gagcagtcta |
| 301 | aagcaaccga gcactgagaa aaatgaactc tgcccaaaga atgtcccaaa gagagagtac |
| 361 | agcgtgaaag aaatcctaaa attggactcc aacccctcca aaggaaagga cctctaccgt |
| 421 | tctaacattt cacccctcac atcagaaaag gacctcgatg actttagaag acgtgggagc |
| 481 | cccgaaatgc ccttctaccc tcgggtcgtt taccccatcc gggcccctct gccagaagac |
| 541 | tttttgaaag cttccctggc ctacgggatc gagagagaac agagaaacag gagaatatgg |
| 601 | gccaaacagg atatctgtgg taagcagttc ctgccccggc tcagggccaa gaacagttgg |
| 661 | aacagcagaa tatgggccaa acaggatatc tgtggtaagc agttcctgcc ccggctcagg |
| 721 | gccaagaaca gatggtcccc agatgcggtc ccgccctcag cagtttctag agaaccatca |
| 781 | gatgtttcca gggtgcccca aggacctgaa atgaccctgt gccttatttg aactaaccaa |
| 841 | tcagttcgct tctcgcttct gttcgcgcgc ttctgctccc cgagctctat ataagcagag |
| 901 | ctcgtttagt gaaccgtcag atcgcctgga gacgccatcc acgctgtttt gacttccata |
| 961 | gaaggatctc gaggccacca tggtgagcaa gggcgaggag ctgttcaccg gggtggtgcc |
| 1021 | catcctggtc gagctggacg gcgacgtaaa cggccacaag ttcagcgtgt ccggcgaggg |
| 1081 | cgagggcgat gccacctacg gcaagctgac cctgaagttc atctgcacca ccggcaagct |
| 1141 | gcccgtgccc tggcccaccc tcgtgaccac cctgacctac ggcgtgcagt gcttcagccg |
| 1201 | ctaccccgac cacatgaagc agcacgactt cttcaagtcc gccatgcccg aaggctacgt |
| 1261 | ccaggagcgc accatcttct tcaaggacga cggcaactac aagacccgcg ccgaggtgaa |
| 1321 | gttcgagggc gacaccctgg tgaaccgcat cgagctgaag ggcatcgact tcaaggagga |
| 1381 | cggcaacatc ctggggcaca agctggagta caactacaac agccacaacg tctatatcat |
| 1441 | ggccgacaag cagaagaacg gcatcaaggt gaacttcaag atccgccaca acatcgagga |
| 1501 | cggcagcgtg cagctcgccg accactacca gcagaacacc cccatcggcg acggccccgt |
| 1561 | gctgctgccc gacaaccact acctgagcac ccagtccgcc ctgagcaaag accccaacga |
| 1621 | gaagcgcgat cacatggtcc tgctggagtt cgtgaccgcc gccgggatca ctctcggcat |
| 1681 | ggacgagctg tacaagtaaa ctagtgtcga ctgctttatt tgtgaaattt gtgatgctat |
| 1741 | tgctttattt gtaaccatta taagctgcaa taaacaagtt aacaacaaca attgcattca |
| 1801 | ttttatgttt caggttcagg gggaggtgtg ggaggttttt taaaacgtac atcactcgct |
| 1861 | cccccattcc atcctccacc actccaagcc cctctgcaag aagcagcccc gaccaaagcc |
| 1921 | tcaagagctc cagccctcac agcagccctg ggaatacggt gtcccctgtg ggccccggct |
| 1981 | ctcaagagca ccgggactcc tacgcttact tgaacgcgtc ctacggcacg gaaggtttgg |
| 2041 | gctcctaccc tggctacgca cccctgcccc acctcccgcc agctttcatc ccctcgtaca |
| 2101 | acgctcacta ccccaagttc ctcttgcccc cctacggcat gaattgtaat ggcctgagcg |
| 2161 | ctgtgagcag catgaatggc atcaacaact ttggcctctt cccgaggctg tgccctgtct |
| 2221 | acagcaatct cctcggtggg ggcatctaga gtagataagt agcatggcgg gttaatcatt |
| 2281 | aactacaagg aacccctagt gatggagttg gccactccct ctctgcgcgc tcgctcgctc |
| 2341 | actgaggccg ggcgaccaaa ggtcgcccga cgcccgggct ttgcccgggc ggcctcagtg |
| 2401 | agcgagcgag cgcgccagct ggcgtaatag cgaagaggcc cgcaccgatc gcccttccca |
| 2461 | acagttgcgc agcctgaatg gcgaatggcg attccgttgc aatggctggc ggtaatattg |
| 2521 | ttctggatat taccagcaag gccgatagtt tgagttcttc tactcaggca agtgatgtta |
| 2581 | ttactaatca aagaagtatt gcgacaacgg ttaatttgcg tgatggacag actcttttac |
| 2641 | tcggtggcct cactgattat aaaaacactt ctcaggattc tggcgtaccg ttcctgtcta |
| 2701 | aaatcccttt aatcggcctc ctgtttagct cccgctctga ttctaacgag gaaagcacgt |
| 2761 | tatacgtgct cgtcaaagca accatagtac gcgccctgta gcggcgcatt aagcgcggcg |
| 2821 | ggtgtggtgg ttacgcgcag cgtgaccgct acacttgcca gcgccctagc gcccgctcct |
| 2881 | ttcgctttct tcccttcctt tctcgccacg ttcgccggct ttccccgtca agctctaaat |
| 2941 | cgggggctcc ctttagggtt ccgatttagt gctttacggc acctcgaccc caaaaaactt |
| 3001 | gattagggtg atggttcacg tagtgggcca tcgccctgat agacggtttt tcgccctttg |
| 3061 | acgttggagt ccacgttctt taatagtgga ctcttgttcc aaactggaac aacactcaac |
| 3121 | cctatctcgg tctattcttt tgatttataa gggattttgc cgatttcggc ctattggtta |
| 3181 | aaaaatgagc tgatttaaca aaaatttaac gcgaatttta acaaaatatt aacgtttaca |
| 3241 | atttaaatat ttgcttatac aatcttcctg tttttggggc ttttctgatt atcaaccggg |
| 3301 | gtacatatga ttgacatgct agttttacga ttaccgttca tcgattctct tgtttgctcc |
| 3361 | agactctcag gcaatgacct gatagccttt gtagagacct ctcaaaaata gctaccctct |
| 3421 | ccggcatgaa tttatcagct agaacggttg aatatcatat tgatggtgat ttgactgtct |
| 3481 | ccggcctttc tcacccgttt gaatctttac ctacacatta ctcaggcatt gcatttaaaa |
| 3541 | tatatgaggg ttctaaaaat ttttatcctt gcgttgaaat aaaggcttct cccgcaaaag |
| 3601 | tattacaggg tcataatgtt tttggtacaa ccgatttagc tttatgctct gaggctttat |
| 3661 | tgcttaattt tgctaattct ttgccttgcc tgtatgattt attggatgtt ggaatcgcct |
| 3721 | gatgcggtat tttctcctta cgcatctgtg cggtatttca caccgcatat ggtgcactct |
| 3781 | cagtacaatc tgctctgatg ccgcatagtt aagccagccc cgacacccgc caacacccgc |
| 3841 | tgacgcgccc tgacgggctt gtctgctccc ggcatccgct tacagacaag ctgtgaccgt |
| 3901 | ctccgggagc tgcatgtgtc agaggttttc accgtcatca ccgaaacgcg cgagacgaaa |
| 3961 | gggcctcgtg atacgcctat ttttataggt taatgtcatg ataataatgg tttcttagac |
| 4021 | gtcaggtggc acttttcggg gaaatgtgcg cggaacccct atttgtttat ttttctaaat |
| 4081 | acattcaaat atgtatccgc tcatgagaca ataaccctga taaatgcttc aataatattg |
| 4141 | aaaaaggaag agtatgagta ttcaacattt ccgtgtcgcc cttattccct tttttgcggc |
| 4201 | attttgcctt cctgtttttg ctcacccaga aacgctggtg aaagtaaaag atgctgaaga |
| 4261 | tcagttgggt gcacgagtgg gttacatcga actggatctc aacagcggta agatccttga |
| 4321 | gagttttcgc cccgaagaac gttttccaat gatgagcact tttaaagttc tgctatgtgg |
| 4381 | cgcggtatta tcccgtattg acgccgggca agagcaactc ggtcgccgca tacactattc |
| 4441 | tcagaatgac ttggttgagt actcaccagt cacagaaaag catcttacgg atggcatgac |
| 4501 | agtaagagaa ttatgcagtg ctgccataac catgagtgat aacactgcgg ccaacttact |
| 4561 | tctgacaacg atcggaggac cgaaggagct aaccgctttt ttgcacaaca tgggggatca |
| 4621 | tgtaactcgc cttgatcgtt gggaaccgga gctgaatgaa gccataccaa acgacgagcg |
| 4681 | tgacaccacg atgcctgtag caatggcaac aacgttgcgc aaactattaa ctggcgaact |
| 4741 | acttactcta gcttcccggc aacaattaat agactggatg gaggcggata aagttgcagg |
| 4801 | accacttctg cgctcggccc ttccggctgg ctggtttatt gctgataaat ctggagccgg |
| 4861 | tgagcgtggg tctcgcggta tcattgcagc actggggcca gatggtaagc cctcccgtat |
| 4921 | cgtagttatc tacacgacgg ggagtcaggc aactatggat gaacgaaata gacagatcgc |
| 4981 | tgagataggt gcctcactga ttaagcattg gtaactgtca gaccaagttt actcatatat |
| 5041 | actttagatt gatttaaaac ttcattttta atttaaaagg atctaggtga agatcctttt |
| 5101 | tgataatctc atgaccaaaa tcccttaacg tgagttttcg ttccactgag cgtcagaccc |
| 5161 | cgtagaaaag atcaaaggat cttcttgaga tccttttttt ctgcgcgtaa tctgctgctt |
| 5221 | gcaaacaaaa aaaccaccgc taccagcggt ggtttgtttg ccggatcaag agctaccaac |
| 5281 | tctttttccg aaggtaactg gcttcagcag agcgcagata ccaaatactg tccttctagt |
| 5341 | gtagccgtag ttaggccacc acttcaagaa ctctgtagca ccgcctacat acctcgctct |
| 5401 | gctaatcctg ttaccagtgg ctgctgccag tggcgataag tcgtgtctta ccgggttgga |
| 5461 | ctcaagacga tagttaccgg ataaggcgca gcggtcgggc tgaacggggg gttcgtgcac |
| 5521 | acagcccagc ttggagcgaa cgacctacac cgaactgaga tacctacagc gtgagctatg |
| 5581 | agaaagcgcc acgcttcccg aagggagaaa ggcggacagg tatccggtaa gcggcagggt |
| 5641 | cggaacagga gagcgcacga gggagcttcc agggggaaac gcctggtatc tttatagtcc |
| 5701 | tgtcgggttt cgccacctct gacttgagcg tcgatttttg tgatgctcgt caggggggcg |
| 5761 | gagcctatgg aaaaacgcca gcaacgcggc ctttttacgg ttcctggcct tttgctggcc |
| 5821 | ttttgctcac atgttctttc ctgcgttatc ccctgattct gtggataacc gtattaccgc |
| 5881 | ctttgagtga gctgataccg ctcgccgcag ccgaacgacc gagcgcagcg agtcagtgag |
| 5941 | cgaggaagcg gaagagcgcc caatacgcaa accgcctctc cccgcgcgtt ggccgattca |
| 6001 | ttaatg |
| // | |
| 1361_pAAV.Blimp1.1.0 kb.MND.GFP.pA | |
| (SEQ ID NO: 54) | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac acgcgtggta |
| 181 | aaccatgaac atcagaaaga cttttattaa cctatgacag ggtccccacc ccagtatttt |
| 241 | tccactccat taaaatggaa gttttttttt tttttttctt ttttgagaca gagttttgct |
| 301 | cttgttgccc agtctggagt gcaatggcac aatctcggct caccacaacc tccacctccc |
| 361 | agattcaagc gattcttctg cctcagcctc ccaagtagct gggattacag gtgtgcgcca |
| 421 | ccacgcccag ctaattttgt atttttagta gagatggggt ttctccatgt tggtcaggct |
| 481 | ggtctcgaac ttccgacctc aggtgatccg cccacctcgg cctcccaaag tgctgggatt |
| 541 | acaggcaaga gccactgcat ccagcttagg ctatcttact ccagcctaaa cagcaatttt |
| 601 | ctatcataag gtctgtacta atgaaaacag aatcacccaa ggctgctgtt tgttctgtct |
| 661 | gtgctgccat tgtccgcatt ttgctgagga ggaaacggaa ctgcactttt gagtgagtgg |
| 721 | cccagagcct tctagaatga gagtgcgttg gaagccagat atgtggcgat tgtgtcgcca |
| 781 | gctgttactc aggttttctc aagaaggagg agcaactttg gcagttttgc ttcagttctc |
| 841 | tctagccctc tgtgtaatcg cccctttttc tttatttcag cacaaacaca gagcagtcta |
| 901 | aagcaaccga gcactgagaa aaatgaactc tgcccaaaga atgtcccaaa gagagagtac |
| 961 | agcgtgaaag aaatcctaaa attggactcc aacccctcca aaggaaagga cctctaccgt |
| 1021 | tctaacattt cacccctcac atcagaaaag gacctcgatg actttagaag acgtgggagc |
| 1081 | cccgaaatgc ccttctaccc tcgggtcgtt taccccatcc gggcccctct gccagaagac |
| 1141 | tttttgaaag cttccctggc ctacgggatc gagagagaac agagaaacag gagaatatgg |
| 1201 | gccaaacagg atatctgtgg taagcagttc ctgccccggc tcagggccaa gaacagttgg |
| 1261 | aacagcagaa tatgggccaa acaggatatc tgtggtaagc agttcctgcc ccggctcagg |
| 1321 | gccaagaaca gatggtcccc agatgcggtc ccgccctcag cagtttctag agaaccatca |
| 1381 | gatgtttcca gggtgcccca aggacctgaa atgaccctgt gccttatttg aactaaccaa |
| 1441 | tcagttcgct tctcgcttct gttcgcgcgc ttctgctccc cgagctctat ataagcagag |
| 1501 | ctcgtttagt gaaccgtcag atcgcctgga gacgccatcc acgctgtttt gacttccata |
| 1561 | gaaggatctc gaggccacca tggtgagcaa gggcgaggag ctgttcaccg gggtggtgcc |
| 1621 | catcctggtc gagctggacg gcgacgtaaa cggccacaag ttcagcgtgt ccggcgaggg |
| 1681 | cgagggcgat gccacctacg gcaagctgac cctgaagttc atctgcacca ccggcaagct |
| 1741 | gcccgtgccc tggcccaccc tcgtgaccac cctgacctac ggcgtgcagt gcttcagccg |
| 1801 | ctaccccgac cacatgaagc agcacgactt cttcaagtcc gccatgcccg aaggctacgt |
| 1861 | ccaggagcgc accatcttct tcaaggacga cggcaactac aagacccgcg ccgaggtgaa |
| 1921 | gttcgagggc gacaccctgg tgaaccgcat cgagctgaag ggcatcgact tcaaggagga |
| 1981 | cggcaacatc ctggggcaca agctggagta caactacaac agccacaacg tctatatcat |
| 2041 | ggccgacaag cagaagaacg gcatcaaggt gaacttcaag atccgccaca acatcgagga |
| 2101 | cggcagcgtg cagctcgccg accactacca gcagaacacc cccatcggcg acggccccgt |
| 2161 | gctgctgccc gacaaccact acctgagcac ccagtccgcc ctgagcaaag accccaacga |
| 2221 | gaagcgcgat cacatggtcc tgctggagtt cgtgaccgcc gccgggatca ctctcggcat |
| 2281 | ggacgagctg tacaagtaaa ctagtgtcga ctgctttatt tgtgaaattt gtgatgctat |
| 2341 | tgctttattt gtaaccatta taagctgcaa taaacaagtt aacaacaaca attgcattca |
| 2401 | ttttatgttt caggttcagg gggaggtgtg ggaggttttt taaaacgtac atcactcgct |
| 2461 | cccccattcc atcctccacc actccaagcc cctctgcaag aagcagcccc gaccaaagcc |
| 2521 | tcaagagctc cagccctcac agcagccctg ggaatacggt gtcccctgtg ggccccggct |
| 2581 | ctcaagagca ccgggactcc tacgcttact tgaacgcgtc ctacggcacg gaaggtttgg |
| 2641 | gctcctaccc tggctacgca cccctgcccc acctcccgcc agctttcatc ccctcgtaca |
| 2701 | acgctcacta ccccaagttc ctcttgcccc cctacggcat gaattgtaat ggcctgagcg |
| 2761 | ctgtgagcag catgaatggc atcaacaact ttggcctctt cccgaggctg tgccctgtct |
| 2821 | acagcaatct cctcggtggg ggcagcctgc cccaccccat gctcaacccc acttctctcc |
| 2881 | cgagctcgct gccctcagat ggagcccgga ggttgctcca gccggagcat cccagggagg |
| 2941 | tgcttgtccc ggcgccccac agtgccttct cctttaccgg ggccgccgcc agcatgaagg |
| 3001 | acaaggcctg tagccccaca agcgggtctc ccacggcggg aacagccgcc acggcagaac |
| 3061 | atgtggtgca gcccaaagct acctcagcag cgatggcagc ccccagcagc gacgaagcca |
| 3121 | tgaatctcat taaaaacaaa agaaacatga ccggctacaa gacccttccc tacccgctga |
| 3181 | agaagcagaa cggcaagatc aagtacgaat gcaacgtttg cgccaagact ttcggccagc |
| 3241 | tctccaatct gaaggtaggc cttgagagag agcagtccaa ggggctgtga gtgcatgctt |
| 3301 | gtgtttgtat ttagcttgct ttccatgggg tatcgattgc atttgcagta gtatgagccc |
| 3361 | ccggttgggg atagtgggta tggattccgc ctggcttttg ccacttctag ctctttgact |
| 3421 | ttggacaagt gacttccctt ctcctctaga gtagataagt agcatggcgg gttaatcatt |
| 3481 | aactacaagg aacccctagt gatggagttg gccactccct ctctgcgcgc tcgctcgctc |
| 3541 | actgaggccg ggcgaccaaa ggtcgcccga cgcccgggct ttgcccgggc ggcctcagtg |
| 3601 | agcgagcgag cgcgccagct ggcgtaatag cgaagaggcc cgcaccgatc gcccttccca |
| 3661 | acagttgcgc agcctgaatg gcgaatggcg attccgttgc aatggctggc ggtaatattg |
| 3721 | ttctggatat taccagcaag gccgatagtt tgagttcttc tactcaggca agtgatgtta |
| 3781 | ttactaatca aagaagtatt gcgacaacgg ttaatttgcg tgatggacag actcttttac |
| 3841 | tcggtggcct cactgattat aaaaacactt ctcaggattc tggcgtaccg ttcctgtcta |
| 3901 | aaatcccttt aatcggcctc ctgtttagct cccgctctga ttctaacgag gaaagcacgt |
| 3961 | tatacgtgct cgtcaaagca accatagtac gcgccctgta gcggcgcatt aagcgcggcg |
| 4021 | ggtgtggtgg ttacgcgcag cgtgaccgct acacttgcca gcgccctagc gcccgctcct |
| 4081 | ttcgctttct tcccttcctt tctcgccacg ttcgccggct ttccccgtca agctctaaat |
| 4141 | cgggggctcc ctttagggtt ccgatttagt gctttacggc acctcgaccc caaaaaactt |
| 4201 | gattagggtg atggttcacg tagtgggcca tcgccctgat agacggtttt tcgccctttg |
| 4261 | acgttggagt ccacgttctt taatagtgga ctcttgttcc aaactggaac aacactcaac |
| 4321 | cctatctcgg tctattcttt tgatttataa gggattttgc cgatttcggc ctattggtta |
| 4381 | aaaaatgagc tgatttaaca aaaatttaac gcgaatttta acaaaatatt aacgtttaca |
| 4441 | atttaaatat ttgcttatac aatcttcctg tttttggggc ttttctgatt atcaaccggg |
| 4501 | gtacatatga ttgacatgct agttttacga ttaccgttca tcgattctct tgtttgctcc |
| 4561 | agactctcag gcaatgacct gatagccttt gtagagacct ctcaaaaata gctaccctct |
| 4621 | ccggcatgaa tttatcagct agaacggttg aatatcatat tgatggtgat ttgactgtct |
| 4681 | ccggcctttc tcacccgttt gaatctttac ctacacatta ctcaggcatt gcatttaaaa |
| 4741 | tatatgaggg ttctaaaaat ttttatcctt gcgttgaaat aaaggcttct cccgcaaaag |
| 4801 | tattacaggg tcataatgtt tttggtacaa ccgatttagc tttatgctct gaggctttat |
| 4861 | tgcttaattt tgctaattct ttgccttgcc tgtatgattt attggatgtt ggaatcgcct |
| 4921 | gatgcggtat tttctcctta cgcatctgtg cggtatttca caccgcatat ggtgcactct |
| 4981 | cagtacaatc tgctctgatg ccgcatagtt aagccagccc cgacacccgc caacacccgc |
| 5041 | tgacgcgccc tgacgggctt gtctgctccc ggcatccgct tacagacaag ctgtgaccgt |
| 5101 | ctccgggagc tgcatgtgtc agaggttttc accgtcatca ccgaaacgcg cgagacgaaa |
| 5161 | gggcctcgtg atacgcctat ttttataggt taatgtcatg ataataatgg tttcttagac |
| 5221 | gtcaggtggc acttttcggg gaaatgtgcg cggaacccct atttgtttat ttttctaaat |
| 5281 | acattcaaat atgtatccgc tcatgagaca ataaccctga taaatgcttc aataatattg |
| 5341 | aaaaaggaag agtatgagta ttcaacattt ccgtgtcgcc cttattccct tttttgcggc |
| 5401 | attttgcctt cctgtttttg ctcacccaga aacgctggtg aaagtaaaag atgctgaaga |
| 5461 | tcagttgggt gcacgagtgg gttacatcga actggatctc aacagcggta agatccttga |
| 5521 | gagttttcgc cccgaagaac gttttccaat gatgagcact tttaaagttc tgctatgtgg |
| 5581 | cgcggtatta tcccgtattg acgccgggca agagcaactc ggtcgccgca tacactattc |
| 5641 | tcagaatgac ttggttgagt actcaccagt cacagaaaag catcttacgg atggcatgac |
| 5701 | agtaagagaa ttatgcagtg ctgccataac catgagtgat aacactgcgg ccaacttact |
| 5761 | tctgacaacg atcggaggac cgaaggagct aaccgctttt ttgcacaaca tgggggatca |
| 5821 | tgtaactcgc cttgatcgtt gggaaccgga gctgaatgaa gccataccaa acgacgagcg |
| 5881 | tgacaccacg atgcctgtag caatggcaac aacgttgcgc aaactattaa ctggcgaact |
| 5941 | acttactcta gcttcccggc aacaattaat agactggatg gaggcggata aagttgcagg |
| 6001 | accacttctg cgctcggccc ttccggctgg ctggtttatt gctgataaat ctggagccgg |
| 6061 | tgagcgtggg tctcgcggta tcattgcagc actggggcca gatggtaagc cctcccgtat |
| 6121 | cgtagttatc tacacgacgg ggagtcaggc aactatggat gaacgaaata gacagatcgc |
| 6181 | tgagataggt gcctcactga ttaagcattg gtaactgtca gaccaagttt actcatatat |
| 6241 | actttagatt gatttaaaac ttcattttta atttaaaagg atctaggtga agatcctttt |
| 6301 | tgataatctc atgaccaaaa tcccttaacg tgagttttcg ttccactgag cgtcagaccc |
| 6361 | cgtagaaaag atcaaaggat cttcttgaga tccttttttt ctgcgcgtaa tctgctgctt |
| 6421 | gcaaacaaaa aaaccaccgc taccagcggt ggtttgtttg ccggatcaag agctaccaac |
| 6481 | tctttttccg aaggtaactg gcttcagcag agcgcagata ccaaatactg tccttctagt |
| 6541 | gtagccgtag ttaggccacc acttcaagaa ctctgtagca ccgcctacat acctcgctct |
| 6601 | gctaatcctg ttaccagtgg ctgctgccag tggcgataag tcgtgtctta ccgggttgga |
| 6661 | ctcaagacga tagttaccgg ataaggcgca gcggtcgggc tgaacggggg gttcgtgcac |
| 6721 | acagcccagc ttggagcgaa cgacctacac cgaactgaga tacctacagc gtgagctatg |
| 6781 | agaaagcgcc acgcttcccg aagggagaaa ggcggacagg tatccggtaa gcggcagggt |
| 6841 | cggaacagga gagcgcacga gggagcttcc agggggaaac gcctggtatc tttatagtcc |
| 6901 | tgtcgggttt cgccacctct gacttgagcg tcgatttttg tgatgctcgt caggggggcg |
| 6961 | gagcctatgg aaaaacgcca gcaacgcggc ctttttacgg ttcctggcct tttgctggcc |
| 7021 | ttttgctcac atgttctttc ctgcgttatc ccctgattct gtggataacc gtattaccgc |
| 7081 | ctttgagtga gctgataccg ctcgccgcag ccgaacgacc gagcgcagcg agtcagtgag |
| 7141 | cgaggaagcg gaagagcgcc caatacgcaa accgcctctc cccgcgcgtt ggccgattca |
| 7201 | ttaatg |
| // | |
| 1366_CCR5.MND.BAFF_CRISPR.HR | |
| LOCUS #1366\CCR5.MND.B 6707 bp DNA circular SYN 10-MAR.-2017 | |
| (SEQ ID NO: 55) | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga ggccaccatg taccggatgc agctgctgag ctgcatcgca |
| 1501 | ctgagcctgg cactggtgac caacagcgca gtgcagggac cagaggagac cgtgacccag |
| 1561 | gactgcctgc agctgatcgc agacagcgag acccccacca tccagaaggg cagctacacc |
| 1621 | ttcgtgccct ggctgctgag cttcaagcgg ggcagcgccc tggaggagaa ggagaacaag |
| 1681 | attctggtga aggagaccgg ctacttcttc atctacggcc aggtgctgta caccgataag |
| 1741 | acctacgcca tgggccacct gatccagcgg aagaaggtgc acgtgttcgg cgacgagctg |
| 1801 | agcctggtga ccctgttccg gtgcatccag aacatgcccg agaccctgcc caacaacagc |
| 1861 | tgctacagcg caggaatcgc aaagctggag gagggcgacg agctgcagct ggcaatcccc |
| 1921 | cgggagaacg cacagatcag cctggacggc gacgtgacct tcttcggcgc cctgaagctc |
| 1981 | ctgtgagtcg actgctttat ttgtgaaatt tgtgatgcta ttgctttatt tgtaaccatt |
| 2041 | ataagctgca ataaacaagt taacaacaac aattgcattc attttatgtt tcaggttcag |
| 2101 | ggggaggtgt gggaggtttt ttaaactcta ttttataggc ttcttctctg gaatcttctt |
| 2161 | catcatcctc ctgacaatcg ataggtacct ggctgtcgtc catgctgtgt ttgctttaaa |
| 2221 | agccaggacg gtcacctttg gggtggtgac aagtgtgatc acttgggtgg tggctgtgtt |
| 2281 | tgcgtctctc ccaggaatca tctttaccag atctcaaaaa gaaggtcttc attacacctg |
| 2341 | cagctctcat tttccataca gtcagtatca attctggaag aatttccaga cattaaagat |
| 2401 | agtcatcttg gggctggtcc tgccgctgct tgtcatggtc atctgctact cgggaatcct |
| 2461 | aaaaactctg cttcggtgtc gaaatgagaa gaagaggcac agggctgtga ggcttatctt |
| 2521 | caccatcatg attgtttatt ttctcttctg ggctccctac aacattgtcc ttctcctgaa |
| 2581 | caccttccag gaattctttg gcctgaataa ttgcagtagc tctaacaggt tggaccaagc |
| 2641 | tatgcaggtg acagagactc ttgggatgac gcactgctgc atcaacccca tcatctatgc |
| 2701 | ctttgtcggg gagaagttca gaaactacct cttagtcttc ttccaaaagc acattgccaa |
| 2761 | acgcttctgc aaatgctgtt ctattttcca gcaagaggct cccgagcgag caagctcagt |
| 2821 | ttacacccga tccactgggg agcaggaaat atctgtgggc ttgtgacacg gactcaagtg |
| 2881 | ggctggtgac ccagtcagag ttgtgcacat ggcttagttt tcatacacac cgcggtctag |
| 2941 | agcatggcta cgtagataag tagcatggcg ggttaatcat taactacaag gaacccctag |
| 3001 | tgatggagtt ggccactccc tctctgcgcg ctcgctcgct cactgaggcc gggcgaccaa |
| 3061 | aggtcgcccg acgcccgggc tttgcccggg cggcctcagt gagcgagcga gcgcgccagc |
| 3121 | tggcgtaata gcgaagaggc ccgcaccgat cgcccttccc aacagttgcg cagcctgaat |
| 3181 | ggcgaatggc gattccgttg caatggctgg cggtaatatt gttctggata ttaccagcaa |
| 3241 | ggccgatagt ttgagttctt ctactcaggc aagtgatgtt attactaatc aaagaagtat |
| 3301 | tgcgacaacg gttaatttgc gtgatggaca gactctttta ctcggtggcc tcactgatta |
| 3361 | taaaaacact tctcaggatt ctggcgtacc gttcctgtct aaaatccctt taatcggcct |
| 3421 | cctgtttagc tcccgctctg attctaacga ggaaagcacg ttatacgtgc tcgtcaaagc |
| 3481 | aaccatagta cgcgccctgt agcggcgcat taagcgcggc gggtgtggtg gttacgcgca |
| 3541 | gcgtgaccgc tacacttgcc agcgccctag cgcccgctcc tttcgctttc ttcccttcct |
| 3601 | ttctcgccac gttcgccggc tttccccgtc aagctctaaa tcgggggctc cctttagggt |
| 3661 | tccgatttag tgctttacgg cacctcgacc ccaaaaaact tgattagggt gatggttcac |
| 3721 | gtagtgggcc atcgccctga tagacggttt ttcgcccttt gacgttggag tccacgttct |
| 3781 | ttaatagtgg actcttgttc caaactggaa caacactcaa ccctatctcg gtctattctt |
| 3841 | ttgatttata agggattttg ccgatttcgg cctattggtt aaaaaatgag ctgatttaac |
| 3901 | aaaaatttaa cgcgaatttt aacaaaatat taacgtttac aatttaaata tttgcttata |
| 3961 | caatcttcct gtttttgggg cttttctgat tatcaaccgg ggtacatatg attgacatgc |
| 4021 | tagttttacg attaccgttc atcgattctc ttgtttgctc cagactctca ggcaatgacc |
| 4081 | tgatagcctt tgtagagacc tctcaaaaat agctaccctc tccggcatga atttatcagc |
| 4141 | tagaacggtt gaatatcata ttgatggtga tttgactgtc tccggccttt ctcacccgtt |
| 4201 | tgaatcttta cctacacatt actcaggcat tgcatttaaa atatatgagg gttctaaaaa |
| 4261 | tttttatcct tgcgttgaaa taaaggcttc tcccgcaaaa gtattacagg gtcataatgt |
| 4321 | ttttggtaca accgatttag ctttatgctc tgaggcttta ttgcttaatt ttgctaattc |
| 4381 | tttgccttgc ctgtatgatt tattggatgt tggaatcgcc tgatgcggta ttttctcctt |
| 4441 | acgcatctgt gcggtatttc acaccgcata tggtgcactc tcagtacaat ctgctctgat |
| 4501 | gccgcatagt taagccagcc ccgacacccg ccaacacccg ctgacgcgcc ctgacgggct |
| 4561 | tgtctgctcc cggcatccgc ttacagacaa gctgtgaccg tctccgggag ctgcatgtgt |
| 4621 | cagaggtttt caccgtcatc accgaaacgc gcgagacgaa agggcctcgt gatacgccta |
| 4681 | tttttatagg ttaatgtcat gataataatg gtttcttaga cgtcaggtgg cacttttcgg |
| 4741 | ggaaatgtgc gcggaacccc tatttgttta tttttctaaa tacattcaaa tatgtatccg |
| 4801 | ctcatgagac aataaccctg ataaatgctt caataatatt gaaaaaggaa gagtatgagt |
| 4861 | attcaacatt tccgtgtcgc ccttattccc ttttttgcgg cattttgcct tcctgttttt |
| 4921 | gctcacccag aaacgctggt gaaagtaaaa gatgctgaag atcagttggg tgcacgagtg |
| 4981 | ggttacatcg aactggatct caacagcggt aagatccttg agagttttcg ccccgaagaa |
| 5041 | cgttttccaa tgatgagcac ttttaaagtt ctgctatgtg gcgcggtatt atcccgtatt |
| 5101 | gacgccgggc aagagcaact cggtcgccgc atacactatt ctcagaatga cttggttgag |
| 5161 | tactcaccag tcacagaaaa gcatcttacg gatggcatga cagtaagaga attatgcagt |
| 5221 | gctgccataa ccatgagtga taacactgcg gccaacttac ttctgacaac gatcggagga |
| 5281 | ccgaaggagc taaccgcttt tttgcacaac atgggggatc atgtaactcg ccttgatcgt |
| 5341 | tgggaaccgg agctgaatga agccatacca aacgacgagc gtgacaccac gatgcctgta |
| 5401 | gcaatggcaa caacgttgcg caaactatta actggcgaac tacttactct agcttcccgg |
| 5461 | caacaattaa tagactggat ggaggcggat aaagttgcag gaccacttct gcgctcggcc |
| 5521 | cttccggctg gctggtttat tgctgataaa tctggagccg gtgagcgtgg gtctcgcggt |
| 5581 | atcattgcag cactggggcc agatggtaag ccctcccgta tcgtagttat ctacacgacg |
| 5641 | gggagtcagg caactatgga tgaacgaaat agacagatcg ctgagatagg tgcctcactg |
| 5701 | attaagcatt ggtaactgtc agaccaagtt tactcatata tactttagat tgatttaaaa |
| 5761 | cttcattttt aatttaaaag gatctaggtg aagatccttt ttgataatct catgaccaaa |
| 5821 | atcccttaac gtgagttttc gttccactga gcgtcagacc ccgtagaaaa gatcaaagga |
| 5881 | tcttcttgag atcctttttt tctgcgcgta atctgctgct tgcaaacaaa aaaaccaccg |
| 5941 | ctaccagcgg tggtttgttt gccggatcaa gagctaccaa ctctttttcc gaaggtaact |
| 6001 | ggcttcagca gagcgcagat accaaatact gtccttctag tgtagccgta gttaggccac |
| 6061 | cacttcaaga actctgtagc accgcctaca tacctcgctc tgctaatcct gttaccagtg |
| 6121 | gctgctgcca gtggcgataa gtcgtgtctt accgggttgg actcaagacg atagttaccg |
| 6181 | gataaggcgc agcggtcggg ctgaacgggg ggttcgtgca cacagcccag cttggagcga |
| 6241 | acgacctaca ccgaactgag atacctacag cgtgagctat gagaaagcgc cacgcttccc |
| 6301 | gaagggagaa aggcggacag gtatccggta agcggcaggg tcggaacagg agagcgcacg |
| 6361 | agggagcttc cagggggaaa cgcctggtat ctttatagtc ctgtcgggtt tcgccacctc |
| 6421 | tgacttgagc gtcgattttt gtgatgctcg tcaggggggc ggagcctatg gaaaaacgcc |
| 6481 | agcaacgcgg cctttttacg gttcctggcc ttttgctggc cttttgctca catgttcttt |
| 6541 | cctgcgttat cccctgattc tgtggataac cgtattaccg cctttgagtg agctgatacc |
| 6601 | gctcgccgca gccgaacgac cgagcgcagc gagtcagtga gcgaggaagc ggaagagcgc |
| 6661 | ccaatacgca aaccgcctct ccccgcgcgt tggccgattc attaatg |
| // | |
| 1367_CCR5.MND.BAFF.2A.GFP | |
| LOCUS #1367\CCR5.MND.B 7494 bp DNA circular SYN 10-MAR.-2017 | |
| (SEQ ID NO: 56) | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga ggccaccatg taccggatgc agctgctgag ctgcatcgca |
| 1501 | ctgagcctgg cactggtgac caacagcgca gtgcagggac cagaggagac cgtgacccag |
| 1561 | gactgcctgc agctgatcgc agacagcgag acccccacca tccagaaggg cagctacacc |
| 1621 | ttcgtgccct ggctgctgag cttcaagcgg ggcagcgccc tggaggagaa ggagaacaag |
| 1681 | attctggtga aggagaccgg ctacttcttc atctacggcc aggtgctgta caccgataag |
| 1741 | acctacgcca tgggccacct gatccagcgg aagaaggtgc acgtgttcgg cgacgagctg |
| 1801 | agcctggtga ccctgttccg gtgcatccag aacatgcccg agaccctgcc caacaacagc |
| 1861 | tgctacagcg caggaatcgc aaagctggag gagggcgacg agctgcagct ggcaatcccc |
| 1921 | cgggagaacg cacagatcag cctggacggc gacgtgacct tcttcggcgc cctgaagctc |
| 1981 | ctgggatccg gtgagggcag aggaagtctt ctaacatgcg gtgacgtgga ggagaatccg |
| 2041 | ggccccatgg tgagcaaggg cgaggagctg ttcaccgggg tggtgcccat cctggtcgag |
| 2101 | ctggacggcg acgtaaacgg ccacaagttc agcgtgtccg gcgagggcga gggcgatgcc |
| 2161 | acctacggca agctgaccct gaagttcatc tgcaccaccg gcaagctgcc cgtgccctgg |
| 2221 | cccaccctcg tgaccaccct gacctacggc gtgcagtgct tcagccgcta ccccgaccac |
| 2281 | atgaagcagc acgacttctt caagtccgcc atgcccgaag gctacgtcca ggagcgcacc |
| 2341 | atcttcttca aggacgacgg caactacaag acccgcgccg aggtgaagtt cgagggcgac |
| 2401 | accctggtga accgcatcga gctgaagggc atcgacttca aggaggacgg caacatcctg |
| 2461 | gggcacaagc tggagtacaa ctacaacagc cacaacgtct atatcatggc cgacaagcag |
| 2521 | aagaacggca tcaaggtgaa cttcaagatc cgccacaaca tcgaggacgg cagcgtgcag |
| 2581 | ctcgccgacc actaccagca gaacaccccc atcggcgacg gccccgtgct gctgcccgac |
| 2641 | aaccactacc tgagcaccca gtccgccctg agcaaagacc ccaacgagaa gcgcgatcac |
| 2701 | atggtcctgc tggagttcgt gaccgccgcc gggatcactc tcggcatgga cgagctgtac |
| 2761 | aagtgaatct agagtcgact gctttatttg tgaaatttgt gatgctattg ctttatttgt |
| 2821 | aaccattata agctgcaata aacaagttaa caacaacaat tgcattcatt ttatgtttca |
| 2881 | ggttcagggg gaggtgtggg aggtttttta aactctattt tataggcttc ttctctggaa |
| 2941 | tcttcttcat catcctcctg acaatcgata ggtacctggc tgtcgtccat gctgtgtttg |
| 3001 | ctttaaaagc caggacggtc acctttgggg tggtgacaag tgtgatcact tgggtggtgg |
| 3061 | ctgtgtttgc gtctctccca ggaatcatct ttaccagatc tcaaaaagaa ggtcttcatt |
| 3121 | acacctgcag ctctcatttt ccatacagtc agtatcaatt ctggaagaat ttccagacat |
| 3181 | taaagatagt catcttgggg ctggtcctgc cgctgcttgt catggtcatc tgctactcgg |
| 3241 | gaatcctaaa aactctgctt cggtgtcgaa atgagaagaa gaggcacagg gctgtgaggc |
| 3301 | ttatcttcac catcatgatt gtttattttc tcttctgggc tccctacaac attgtccttc |
| 3361 | tcctgaacac cttccaggaa ttctttggcc tgaataattg cagtagctct aacaggttgg |
| 3421 | accaagctat gcaggtgaca gagactcttg ggatgacgca ctgctgcatc aaccccatca |
| 3481 | tctatgcctt tgtcggggag aagttcagaa actacctctt agtcttcttc caaaagcaca |
| 3541 | ttgccaaacg cttctgcaaa tgctgttcta ttttccagca agaggctccc gagcgagcaa |
| 3601 | gctcagttta cacccgatcc actggggagc aggaaatatc tgtgggcttg tgacacggac |
| 3661 | tcaagtgggc tggtgaccca gtcagagttg tgcacatggc ttagttttca tacacaccgc |
| 3721 | ggtctagagc atggctacgt agataagtag catggcgggt taatcattaa ctacaaggaa |
| 3781 | cccctagtga tggagttggc cactccctct ctgcgcgctc gctcgctcac tgaggccggg |
| 3841 | cgaccaaagg tcgcccgacg cccgggcttt gcccgggcgg cctcagtgag cgagcgagcg |
| 3901 | cgccagctgg cgtaatagcg aagaggcccg caccgatcgc ccttcccaac agttgcgcag |
| 3961 | cctgaatggc gaatggcgat tccgttgcaa tggctggcgg taatattgtt ctggatatta |
| 4021 | ccagcaaggc cgatagtttg agttcttcta ctcaggcaag tgatgttatt actaatcaaa |
| 4081 | gaagtattgc gacaacggtt aatttgcgtg atggacagac tcttttactc ggtggcctca |
| 4141 | ctgattataa aaacacttct caggattctg gcgtaccgtt cctgtctaaa atccctttaa |
| 4201 | tcggcctcct gtttagctcc cgctctgatt ctaacgagga aagcacgtta tacgtgctcg |
| 4261 | tcaaagcaac catagtacgc gccctgtagc ggcgcattaa gcgcggcggg tgtggtggtt |
| 4321 | acgcgcagcg tgaccgctac acttgccagc gccctagcgc ccgctccttt cgctttcttc |
| 4381 | ccttcctttc tcgccacgtt cgccggcttt ccccgtcaag ctctaaatcg ggggctccct |
| 4441 | ttagggttcc gatttagtgc tttacggcac ctcgacccca aaaaacttga ttagggtgat |
| 4501 | ggttcacgta gtgggccatc gccctgatag acggtttttc gccctttgac gttggagtcc |
| 4561 | acgttcttta atagtggact cttgttccaa actggaacaa cactcaaccc tatctcggtc |
| 4621 | tattcttttg atttataagg gattttgccg atttcggcct attggttaaa aaatgagctg |
| 4681 | atttaacaaa aatttaacgc gaattttaac aaaatattaa cgtttacaat ttaaatattt |
| 4741 | gcttatacaa tcttcctgtt tttggggctt ttctgattat caaccggggt acatatgatt |
| 4801 | gacatgctag ttttacgatt accgttcatc gattctcttg tttgctccag actctcaggc |
| 4861 | aatgacctga tagcctttgt agagacctct caaaaatagc taccctctcc ggcatgaatt |
| 4921 | tatcagctag aacggttgaa tatcatattg atggtgattt gactgtctcc ggcctttctc |
| 4981 | acccgtttga atctttacct acacattact caggcattgc atttaaaata tatgagggtt |
| 5041 | ctaaaaattt ttatccttgc gttgaaataa aggcttctcc cgcaaaagta ttacagggtc |
| 5101 | ataatgtttt tggtacaacc gatttagctt tatgctctga ggctttattg cttaattttg |
| 5161 | ctaattcttt gccttgcctg tatgatttat tggatgttgg aatcgcctga tgcggtattt |
| 5221 | tctccttacg catctgtgcg gtatttcaca ccgcatatgg tgcactctca gtacaatctg |
| 5281 | ctctgatgcc gcatagttaa gccagccccg acacccgcca acacccgctg acgcgccctg |
| 5341 | acgggcttgt ctgctcccgg catccgctta cagacaagct gtgaccgtct ccgggagctg |
| 5401 | catgtgtcag aggttttcac cgtcatcacc gaaacgcgcg agacgaaagg gcctcgtgat |
| 5461 | acgcctattt ttataggtta atgtcatgat aataatggtt tcttagacgt caggtggcac |
| 5521 | ttttcgggga aatgtgcgcg gaacccctat ttgtttattt ttctaaatac attcaaatat |
| 5581 | gtatccgctc atgagacaat aaccctgata aatgcttcaa taatattgaa aaaggaagag |
| 5641 | tatgagtatt caacatttcc gtgtcgccct tattcccttt tttgcggcat tttgccttcc |
| 5701 | tgtttttgct cacccagaaa cgctggtgaa agtaaaagat gctgaagatc agttgggtgc |
| 5761 | acgagtgggt tacatcgaac tggatctcaa cagcggtaag atccttgaga gttttcgccc |
| 5821 | cgaagaacgt tttccaatga tgagcacttt taaagttctg ctatgtggcg cggtattatc |
| 5881 | ccgtattgac gccgggcaag agcaactcgg tcgccgcata cactattctc agaatgactt |
| 5941 | ggttgagtac tcaccagtca cagaaaagca tcttacggat ggcatgacag taagagaatt |
| 6001 | atgcagtgct gccataacca tgagtgataa cactgcggcc aacttacttc tgacaacgat |
| 6061 | cggaggaccg aaggagctaa ccgctttttt gcacaacatg ggggatcatg taactcgcct |
| 6121 | tgatcgttgg gaaccggagc tgaatgaagc cataccaaac gacgagcgtg acaccacgat |
| 6181 | gcctgtagca atggcaacaa cgttgcgcaa actattaact ggcgaactac ttactctagc |
| 6241 | ttcccggcaa caattaatag actggatgga ggcggataaa gttgcaggac cacttctgcg |
| 6301 | ctcggccctt ccggctggct ggtttattgc tgataaatct ggagccggtg agcgtgggtc |
| 6361 | tcgcggtatc attgcagcac tggggccaga tggtaagccc tcccgtatcg tagttatcta |
| 6421 | cacgacgggg agtcaggcaa ctatggatga acgaaataga cagatcgctg agataggtgc |
| 6481 | ctcactgatt aagcattggt aactgtcaga ccaagtttac tcatatatac tttagattga |
| 6541 | tttaaaactt catttttaat ttaaaaggat ctaggtgaag atcctttttg ataatctcat |
| 6601 | gaccaaaatc ccttaacgtg agttttcgtt ccactgagcg tcagaccccg tagaaaagat |
| 6661 | caaaggatct tcttgagatc ctttttttct gcgcgtaatc tgctgcttgc aaacaaaaaa |
| 6721 | accaccgcta ccagcggtgg tttgtttgcc ggatcaagag ctaccaactc tttttccgaa |
| 6781 | ggtaactggc ttcagcagag cgcagatacc aaatactgtc cttctagtgt agccgtagtt |
| 6841 | aggccaccac ttcaagaact ctgtagcacc gcctacatac ctcgctctgc taatcctgtt |
| 6901 | accagtggct gctgccagtg gcgataagtc gtgtcttacc gggttggact caagacgata |
| 6961 | gttaccggat aaggcgcagc ggtcgggctg aacggggggt tcgtgcacac agcccagctt |
| 7021 | ggagcgaacg acctacaccg aactgagata cctacagcgt gagctatgag aaagcgccac |
| 7081 | gcttcccgaa gggagaaagg cggacaggta tccggtaagc ggcagggtcg gaacaggaga |
| 7141 | gcgcacgagg gagcttccag ggggaaacgc ctggtatctt tatagtcctg tcgggtttcg |
| 7201 | ccacctctga cttgagcgtc gatttttgtg atgctcgtca ggggggcgga gcctatggaa |
| 7261 | aaacgccagc aacgcggcct ttttacggtt cctggccttt tgctggcctt ttgctcacat |
| 7321 | gttctttcct gcgttatccc ctgattctgt ggataaccgt attaccgcct ttgagtgagc |
| 7381 | tgataccgct cgccgcagcc gaacgaccga gcgcagcgag tcagtgagcg aggaagcgga |
| 7441 | agagcgccca atacgcaaac cgcctctccc cgcgcgttgg ccgattcatt aatg |
| // | |
| 1378_CCR5.MND.mCherry.2A.coFIXpadua.WPRE.pa | |
| LOCUS #1378 8330 bp DNA circular UNA 09-AUG.-2017 | |
| DEFINITION Gibson Assembly of mCherry T2A - coFIXpadua into 1367 XhoI + | |
| SalI (6.2 kb). | |
| (SEQ ID NO: 57) | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga ggccaccatg gtgagcaagg gcgaggagga taacatggcc |
| 1501 | atcatcaagg agttcatgcg cttcaaggtg cacatggagg gctccgtgaa cggccacgag |
| 1561 | ttcgagatcg agggcgaggg cgagggccgc ccctacgagg gcacccagac cgccaagctg |
| 1621 | aaggtgacca agggtggccc cctgcccttc gcctgggaca tcctgtcccc tcagttcatg |
| 1681 | tacggctcca aggcctacgt gaagcacccc gccgacatcc ccgactactt gaagctgtcc |
| 1741 | ttccccgagg gcttcaagtg ggagcgcgtg atgaacttcg aggacggcgg cgtggtgacc |
| 1801 | gtgacccagg actcctctct gcaggacggc gagttcatct acaaggtgaa gctgcgcggc |
| 1861 | accaacttcc cctccgacgg ccccgtaatg cagaagaaga ccatgggctg ggaggcctcc |
| 1921 | tccgagcgga tgtaccccga ggacggcgcc ctgaagggcg agatcaagca gaggctgaag |
| 1981 | ctgaaggacg gcggccacta cgacgctgag gtcaagacca cctacaaggc caagaagccc |
| 2041 | gtgcagctgc ccggcgccta caacgtcaac atcaagttgg acatcacctc ccacaacgag |
| 2101 | gactacacca tcgtggaaca gtacgaacgc gccgagggcc gccactccac cggcggcatg |
| 2161 | gacgagctgt acaagggatc cggtgagggc agaggaagtc ttctaacatg cggtgacgtg |
| 2221 | gaggagaatc cgggccccat gatcatggcc gagagccctg gcctgatcac catctgcctg |
| 2281 | ctgggctacc tgctgagcgc cgagtgcacc gtgttcctgg accacgagaa cgccaacaag |
| 2341 | atcctgaacc ggcccaagag atacaacagc ggcaagctgg aggagttcgt gcagggcaac |
| 2401 | ctggagaggg agtgcatgga ggagaagtgc agcttcgagg aggccaggga agtgttcgag |
| 2461 | aacaccgagc ggaccaccga gttctggaag cagtacgtgg acggcgacca gtgcgagagc |
| 2521 | aacccttgcc tgaacggcgg cagctgcaag gacgacatca acagctacga gtgctggtgc |
| 2581 | cctttcggct tcgagggcaa gaactgcgag ctggacgtga cctgcaacat caagaacggc |
| 2641 | cgctgcgagc agttctgcaa gaacagcgcc gacaacaaag tggtgtgtag ctgcaccgag |
| 2701 | ggctacagac tggccgagaa ccagaagagc tgcgagcccg ccgtgccctt cccctgcggc |
| 2761 | agagtgagcg tgtcccagac cagcaagctg accagagccg agaccgtgtt ccccgacgtg |
| 2821 | gactacgtga atagcaccga ggccgagacc atcctggaca acatcaccca gagcacccag |
| 2881 | tccttcaacg acttcaccag agttgtgggc ggcgaggacg ccaagcccgg ccagttcccc |
| 2941 | tggcaggtgg tgctgaacgg caaagtggat gccttctgcg gcggcagcat cgtgaacgag |
| 3001 | aagtggatcg tgacagccgc ccactgcgtg gagaccggcg tgaagatcac cgtggtggcc |
| 3061 | ggcgaacaca atatcgagga gaccgagcac accgagcaga agcggaacgt catccggatt |
| 3121 | atcccccacc acaactacaa cgccgccatc aacaagtaca accacgacat cgccctgctg |
| 3181 | gagctggacg agcctctggt gctgaatagc tacgtgaccc ccatctgcat cgccgacaag |
| 3241 | gagtacacca acatcttcct gaagttcggc agcggctacg tgtccggctg gggcagagtg |
| 3301 | ttccacaagg gcagaagcgc cctggtgctg cagtacctga gagtgcccct ggtggacaga |
| 3361 | gccacctgcc tgttgagcac caagttcacc atctacaaca acatgttctg cgccggcttc |
| 3421 | cacgagggcg gcagagacag ctgccagggc gacagcggcg gaccccacgt gaccgaagtg |
| 3481 | gagggcacca gcttcctgac cggcatcatc agctggggcg aggagtgcgc catgaagggc |
| 3541 | aagtacggca tctacaccaa agtgagccgg tacgtgaact ggatcaagga gaaaaccaag |
| 3601 | ctgacctgag tcgactgctt tatttgtgaa atttgtgatg ctattgcttt atttgtaacc |
| 3661 | attataagct gcaataaaca agttaacaac aacaattgca ttcattttat gtttcaggtt |
| 3721 | cagggggagg tgtgggaggt tttttaaact ctattttata ggcttcttct ctggaatctt |
| 3781 | cttcatcatc ctcctgacaa tcgataggta cctggctgtc gtccatgctg tgtttgcttt |
| 3841 | aaaagccagg acggtcacct ttggggtggt gacaagtgtg atcacttggg tggtggctgt |
| 3901 | gtttgcgtct ctcccaggaa tcatctttac cagatctcaa aaagaaggtc ttcattacac |
| 3961 | ctgcagctct cattttccat acagtcagta tcaattctgg aagaatttcc agacattaaa |
| 4021 | gatagtcatc ttggggctgg tcctgccgct gcttgtcatg gtcatctgct actcgggaat |
| 4081 | cctaaaaact ctgcttcggt gtcgaaatga gaagaagagg cacagggctg tgaggcttat |
| 4141 | cttcaccatc atgattgttt attttctctt ctgggctccc tacaacattg tccttctcct |
| 4201 | gaacaccttc caggaattct ttggcctgaa taattgcagt agctctaaca ggttggacca |
| 4261 | agctatgcag gtgacagaga ctcttgggat gacgcactgc tgcatcaacc ccatcatcta |
| 4321 | tgcctttgtc ggggagaagt tcagaaacta cctcttagtc ttcttccaaa agcacattgc |
| 4381 | caaacgcttc tgcaaatgct gttctatttt ccagcaagag gctcccgagc gagcaagctc |
| 4441 | agtttacacc cgatccactg gggagcagga aatatctgtg ggcttgtgac acggactcaa |
| 4501 | gtgggctggt gacccagtca gagttgtgca catggcttag ttttcataca caccgcggtc |
| 4561 | tagagcatgg ctacgtagat aagtagcatg gcgggttaat cattaactac aaggaacccc |
| 4621 | tagtgatgga gttggccact ccctctctgc gcgctcgctc gctcactgag gccgggcgac |
| 4681 | caaaggtcgc ccgacgcccg ggctttgccc gggcggcctc agtgagcgag cgagcgcgcc |
| 4741 | agctggcgta atagcgaaga ggcccgcacc gatcgccctt cccaacagtt gcgcagcctg |
| 4801 | aatggcgaat ggcgattccg ttgcaatggc tggcggtaat attgttctgg atattaccag |
| 4861 | caaggccgat agtttgagtt cttctactca ggcaagtgat gttattacta atcaaagaag |
| 4921 | tattgcgaca acggttaatt tgcgtgatgg acagactctt ttactcggtg gcctcactga |
| 4981 | ttataaaaac acttctcagg attctggcgt accgttcctg tctaaaatcc ctttaatcgg |
| 5041 | cctcctgttt agctcccgct ctgattctaa cgaggaaagc acgttatacg tgctcgtcaa |
| 5101 | agcaaccata gtacgcgccc tgtagcggcg cattaagcgc ggcgggtgtg gtggttacgc |
| 5161 | gcagcgtgac cgctacactt gccagcgccc tagcgcccgc tcctttcgct ttcttccctt |
| 5221 | cctttctcgc cacgttcgcc ggctttcccc gtcaagctct aaatcggggg ctccctttag |
| 5281 | ggttccgatt tagtgcttta cggcacctcg accccaaaaa acttgattag ggtgatggtt |
| 5341 | cacgtagtgg gccatcgccc tgatagacgg tttttcgccc tttgacgttg gagtccacgt |
| 5401 | tctttaatag tggactcttg ttccaaactg gaacaacact caaccctatc tcggtctatt |
| 5461 | cttttgattt ataagggatt ttgccgattt cggcctattg gttaaaaaat gagctgattt |
| 5521 | aacaaaaatt taacgcgaat tttaacaaaa tattaacgtt tacaatttaa atatttgctt |
| 5581 | atacaatctt cctgtttttg gggcttttct gattatcaac cggggtacat atgattgaca |
| 5641 | tgctagtttt acgattaccg ttcatcgatt ctcttgtttg ctccagactc tcaggcaatg |
| 5701 | acctgatagc ctttgtagag acctctcaaa aatagctacc ctctccggca tgaatttatc |
| 5761 | agctagaacg gttgaatatc atattgatgg tgatttgact gtctccggcc tttctcaccc |
| 5821 | gtttgaatct ttacctacac attactcagg cattgcattt aaaatatatg agggttctaa |
| 5881 | aaatttttat ccttgcgttg aaataaaggc ttctcccgca aaagtattac agggtcataa |
| 5941 | tgtttttggt acaaccgatt tagctttatg ctctgaggct ttattgctta attttgctaa |
| 6001 | ttctttgcct tgcctgtatg atttattgga tgttggaatc gcctgatgcg gtattttctc |
| 6061 | cttacgcatc tgtgcggtat ttcacaccgc atatggtgca ctctcagtac aatctgctct |
| 6121 | gatgccgcat agttaagcca gccccgacac ccgccaacac ccgctgacgc gccctgacgg |
| 6181 | gcttgtctgc tcccggcatc cgcttacaga caagctgtga ccgtctccgg gagctgcatg |
| 6241 | tgtcagaggt tttcaccgtc atcaccgaaa cgcgcgagac gaaagggcct cgtgatacgc |
| 6301 | ctatttttat aggttaatgt catgataata atggtttctt agacgtcagg tggcactttt |
| 6361 | cggggaaatg tgcgcggaac ccctatttgt ttatttttct aaatacattc aaatatgtat |
| 6421 | ccgctcatga gacaataacc ctgataaatg cttcaataat attgaaaaag gaagagtatg |
| 6481 | agtattcaac atttccgtgt cgcccttatt cccttttttg cggcattttg ccttcctgtt |
| 6541 | tttgctcacc cagaaacgct ggtgaaagta aaagatgctg aagatcagtt gggtgcacga |
| 6601 | gtgggttaca tcgaactgga tctcaacagc ggtaagatcc ttgagagttt tcgccccgaa |
| 6661 | gaacgttttc caatgatgag cacttttaaa gttctgctat gtggcgcggt attatcccgt |
| 6721 | attgacgccg ggcaagagca actcggtcgc cgcatacact attctcagaa tgacttggtt |
| 6781 | gagtactcac cagtcacaga aaagcatctt acggatggca tgacagtaag agaattatgc |
| 6841 | agtgctgcca taaccatgag tgataacact gcggccaact tacttctgac aacgatcgga |
| 6901 | ggaccgaagg agctaaccgc ttttttgcac aacatggggg atcatgtaac tcgccttgat |
| 6961 | cgttgggaac cggagctgaa tgaagccata ccaaacgacg agcgtgacac cacgatgcct |
| 7021 | gtagcaatgg caacaacgtt gcgcaaacta ttaactggcg aactacttac tctagcttcc |
| 7081 | cggcaacaat taatagactg gatggaggcg gataaagttg caggaccact tctgcgctcg |
| 7141 | gcccttccgg ctggctggtt tattgctgat aaatctggag ccggtgagcg tgggtctcgc |
| 7201 | ggtatcattg cagcactggg gccagatggt aagccctccc gtatcgtagt tatctacacg |
| 7261 | acggggagtc aggcaactat ggatgaacga aatagacaga tcgctgagat aggtgcctca |
| 7321 | ctgattaagc attggtaact gtcagaccaa gtttactcat atatacttta gattgattta |
| 7381 | aaacttcatt tttaatttaa aaggatctag gtgaagatcc tttttgataa tctcatgacc |
| 7441 | aaaatccctt aacgtgagtt ttcgttccac tgagcgtcag accccgtaga aaagatcaaa |
| 7501 | ggatcttctt gagatccttt ttttctgcgc gtaatctgct gcttgcaaac aaaaaaacca |
| 7561 | ccgctaccag cggtggtttg tttgccggat caagagctac caactctttt tccgaaggta |
| 7621 | actggcttca gcagagcgca gataccaaat actgtccttc tagtgtagcc gtagttaggc |
| 7681 | caccacttca agaactctgt agcaccgcct acatacctcg ctctgctaat cctgttacca |
| 7741 | gtggctgctg ccagtggcga taagtcgtgt cttaccgggt tggactcaag acgatagtta |
| 7801 | ccggataagg cgcagcggtc gggctgaacg gggggttcgt gcacacagcc cagcttggag |
| 7861 | cgaacgacct acaccgaact gagataccta cagcgtgagc tatgagaaag cgccacgctt |
| 7921 | cccgaaggga gaaaggcgga caggtatccg gtaagcggca gggtcggaac aggagagcgc |
| 7981 | acgagggagc ttccaggggg aaacgcctgg tatctttata gtcctgtcgg gtttcgccac |
| 8041 | ctctgacttg agcgtcgatt tttgtgatgc tcgtcagggg ggcggagcct atggaaaaac |
| 8101 | gccagcaacg cggccttttt acggttcctg gccttttgct ggccttttgc tcacatgttc |
| 8161 | tttcctgcgt tatcccctga ttctgtggat aaccgtatta ccgcctttga gtgagctgat |
| 8221 | accgctcgcc gcagccgaac gaccgagcgc agcgagtcag tgagcgagga agcggaagag |
| 8281 | cgcccaatac gcaaaccgcc tctccccgcg cgttggccga ttcattaatg |
| // | |
| 1376_CCR5.MND.FiX.coFIXpadua.WPRE.pA | |
| LOCUS (#1376)\CCR5.MND 7806 bp DNA circular SYN 09-AUG.-2017 | |
| (SEQ ID NO: 58) | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga gatgatcatg gccgagagcc ctggcctgat caccatctgc |
| 1501 | ctgctgggct acctgctgag cgccgagtgc accgtgttcc tggaccacga gaacgccaac |
| 1561 | aagatcctga accggcccaa gagatacaac agcggcaagc tggaggagtt cgtgcagggc |
| 1621 | aacctggaga gggagtgcat ggaggagaag tgcagcttcg aggaggccag ggaagtgttc |
| 1681 | gagaacaccg agcggaccac cgagttctgg aagcagtacg tggacggcga ccagtgcgag |
| 1741 | agcaaccctt gcctgaacgg cggcagctgc aaggacgaca tcaacagcta cgagtgctgg |
| 1801 | tgccctttcg gcttcgaggg caagaactgc gagctggacg tgacctgcaa catcaagaac |
| 1861 | ggccgctgcg agcagttctg caagaacagc gccgacaaca aagtggtgtg tagctgcacc |
| 1921 | gagggctaca gactggccga gaaccagaag agctgcgagc ccgccgtgcc cttcccctgc |
| 1981 | ggcagagtga gcgtgtccca gaccagcaag ctgaccagag ccgagaccgt gttccccgac |
| 2041 | gtggactacg tgaatagcac cgaggccgag accatcctgg acaacatcac ccagagcacc |
| 2101 | cagtccttca acgacttcac cagagttgtg ggcggcgagg acgccaagcc cggccagttc |
| 2161 | ccctggcagg tggtgctgaa cggcaaagtg gatgccttct gcggcggcag catcgtgaac |
| 2221 | gagaagtgga tcgtgacagc cgcccactgc gtggagaccg gcgtgaagat caccgtggtg |
| 2281 | gccggcgaac acaatatcga ggagaccgag cacaccgagc agaagcggaa cgtcatccgg |
| 2341 | attatccccc accacaacta caacgccgcc atcaacaagt acaaccacga catcgccctg |
| 2401 | ctggagctgg acgagcctct ggtgctgaat agctacgtga cccccatctg catcgccgac |
| 2461 | aaggagtaca ccaacatctt cctgaagttc ggcagcggct acgtgtccgg ctggggcaga |
| 2521 | gtgttccaca agggcagaag cgccctggtg ctgcagtacc tgagagtgcc cctggtggac |
| 2581 | agagccacct gcctgttgag caccaagttc accatctaca acaacatgtt ctgcgccggc |
| 2641 | ttccacgagg gcggcagaga cagctgccag ggcgacagcg gcggacccca cgtgaccgaa |
| 2701 | gtggagggca ccagcttcct gaccggcatc atcagctggg gcgaggagtg cgccatgaag |
| 2761 | ggcaagtacg gcatctacac caaagtgagc cggtacgtga actggatcaa ggagaaaacc |
| 2821 | aagctgacct gagtcgacga taatcaacct ctggattaca aaatttgtga aagattgact |
| 2881 | ggtattctta actatgttgc tccttttacg ctatgtggat acgctgcttt aatgcctttg |
| 2941 | tatcatgcta ttgcttcccg tatggctttc attttctcct ccttgtataa atcctggtta |
| 3001 | gttcttgcca cggcggaact catcgccgcc tgccttgccc gctgctggac aggggctcgg |
| 3061 | ctgttgggca ctgacaattc cgtgggtcga ctgctttatt tgtgaaattt gtgatgctat |
| 3121 | tgctttattt gtaaccatta taagctgcaa taaacaagtt aacaacaaca attgcattca |
| 3181 | ttttatgttt caggttcagg gggaggtgtg ggaggttttt taaactctat tttataggct |
| 3241 | tcttctctgg aatcttcttc atcatcctcc tgacaatcga taggtacctg gctgtcgtcc |
| 3301 | atgctgtgtt tgctttaaaa gccaggacgg tcacctttgg ggtggtgaca agtgtgatca |
| 3361 | cttgggtggt ggctgtgttt gcgtctctcc caggaatcat ctttaccaga tctcaaaaag |
| 3421 | aaggtcttca ttacacctgc agctctcatt ttccatacag tcagtatcaa ttctggaaga |
| 3481 | atttccagac attaaagata gtcatcttgg ggctggtcct gccgctgctt gtcatggtca |
| 3541 | tctgctactc gggaatccta aaaactctgc ttcggtgtcg aaatgagaag aagaggcaca |
| 3601 | gggctgtgag gcttatcttc accatcatga ttgtttattt tctcttctgg gctccctaca |
| 3661 | acattgtcct tctcctgaac accttccagg aattctttgg cctgaataat tgcagtagct |
| 3721 | ctaacaggtt ggaccaagct atgcaggtga cagagactct tgggatgacg cactgctgca |
| 3781 | tcaaccccat catctatgcc tttgtcgggg agaagttcag aaactacctc ttagtcttct |
| 3841 | tccaaaagca cattgccaaa cgcttctgca aatgctgttc tattttccag caagaggctc |
| 3901 | ccgagcgagc aagctcagtt tacacccgat ccactgggga gcaggaaata tctgtgggct |
| 3961 | tgtgacacgg actcaagtgg gctggtgacc cagtcagagt tgtgcacatg gcttagtttt |
| 4021 | catacacacc gcggtctaga gcatggctac gtagataagt agcatggcgg gttaatcatt |
| 4081 | aactacaagg aacccctagt gatggagttg gccactccct ctctgcgcgc tcgctcgctc |
| 4141 | actgaggccg ggcgaccaaa ggtcgcccga cgcccgggct ttgcccgggc ggcctcagtg |
| 4201 | agcgagcgag cgcgccagct ggcgtaatag cgaagaggcc cgcaccgatc gcccttccca |
| 4261 | acagttgcgc agcctgaatg gcgaatggcg attccgttgc aatggctggc ggtaatattg |
| 4321 | ttctggatat taccagcaag gccgatagtt tgagttcttc tactcaggca agtgatgtta |
| 4381 | ttactaatca aagaagtatt gcgacaacgg ttaatttgcg tgatggacag actcttttac |
| 4441 | tcggtggcct cactgattat aaaaacactt ctcaggattc tggcgtaccg ttcctgtcta |
| 4501 | aaatcccttt aatcggcctc ctgtttagct cccgctctga ttctaacgag gaaagcacgt |
| 4561 | tatacgtgct cgtcaaagca accatagtac gcgccctgta gcggcgcatt aagcgcggcg |
| 4621 | ggtgtggtgg ttacgcgcag cgtgaccgct acacttgcca gcgccctagc gcccgctcct |
| 4681 | ttcgctttct tcccttcctt tctcgccacg ttcgccggct ttccccgtca agctctaaat |
| 4741 | cgggggctcc ctttagggtt ccgatttagt gctttacggc acctcgaccc caaaaaactt |
| 4801 | gattagggtg atggttcacg tagtgggcca tcgccctgat agacggtttt tcgccctttg |
| 4861 | acgttggagt ccacgttctt taatagtgga ctcttgttcc aaactggaac aacactcaac |
| 4921 | cctatctcgg tctattcttt tgatttataa gggattttgc cgatttcggc ctattggtta |
| 4981 | aaaaatgagc tgatttaaca aaaatttaac gcgaatttta acaaaatatt aacgtttaca |
| 5041 | atttaaatat ttgcttatac aatcttcctg tttttggggc ttttctgatt atcaaccggg |
| 5101 | gtacatatga ttgacatgct agttttacga ttaccgttca tcgattctct tgtttgctcc |
| 5161 | agactctcag gcaatgacct gatagccttt gtagagacct ctcaaaaata gctaccctct |
| 5221 | ccggcatgaa tttatcagct agaacggttg aatatcatat tgatggtgat ttgactgtct |
| 5281 | ccggcctttc tcacccgttt gaatctttac ctacacatta ctcaggcatt gcatttaaaa |
| 5341 | tatatgaggg ttctaaaaat ttttatcctt gcgttgaaat aaaggcttct cccgcaaaag |
| 5401 | tattacaggg tcataatgtt tttggtacaa ccgatttagc tttatgctct gaggctttat |
| 5461 | tgcttaattt tgctaattct ttgccttgcc tgtatgattt attggatgtt ggaatcgcct |
| 5521 | gatgcggtat tttctcctta cgcatctgtg cggtatttca caccgcatat ggtgcactct |
| 5581 | cagtacaatc tgctctgatg ccgcatagtt aagccagccc cgacacccgc caacacccgc |
| 5641 | tgacgcgccc tgacgggctt gtctgctccc ggcatccgct tacagacaag ctgtgaccgt |
| 5701 | ctccgggagc tgcatgtgtc agaggttttc accgtcatca ccgaaacgcg cgagacgaaa |
| 5761 | gggcctcgtg atacgcctat ttttataggt taatgtcatg ataataatgg tttcttagac |
| 5821 | gtcaggtggc acttttcggg gaaatgtgcg cggaacccct atttgtttat ttttctaaat |
| 5881 | acattcaaat atgtatccgc tcatgagaca ataaccctga taaatgcttc aataatattg |
| 5941 | aaaaaggaag agtatgagta ttcaacattt ccgtgtcgcc cttattccct tttttgcggc |
| 6001 | attttgcctt cctgtttttg ctcacccaga aacgctggtg aaagtaaaag atgctgaaga |
| 6061 | tcagttgggt gcacgagtgg gttacatcga actggatctc aacagcggta agatccttga |
| 6121 | gagttttcgc cccgaagaac gttttccaat gatgagcact tttaaagttc tgctatgtgg |
| 6181 | cgcggtatta tcccgtattg acgccgggca agagcaactc ggtcgccgca tacactattc |
| 6241 | tcagaatgac ttggttgagt actcaccagt cacagaaaag catcttacgg atggcatgac |
| 6301 | agtaagagaa ttatgcagtg ctgccataac catgagtgat aacactgcgg ccaacttact |
| 6361 | tctgacaacg atcggaggac cgaaggagct aaccgctttt ttgcacaaca tgggggatca |
| 6421 | tgtaactcgc cttgatcgtt gggaaccgga gctgaatgaa gccataccaa acgacgagcg |
| 6481 | tgacaccacg atgcctgtag caatggcaac aacgttgcgc aaactattaa ctggcgaact |
| 6541 | acttactcta gcttcccggc aacaattaat agactggatg gaggcggata aagttgcagg |
| 6601 | accacttctg cgctcggccc ttccggctgg ctggtttatt gctgataaat ctggagccgg |
| 6661 | tgagcgtggg tctcgcggta tcattgcagc actggggcca gatggtaagc cctcccgtat |
| 6721 | cgtagttatc tacacgacgg ggagtcaggc aactatggat gaacgaaata gacagatcgc |
| 6781 | tgagataggt gcctcactga ttaagcattg gtaactgtca gaccaagttt actcatatat |
| 6841 | actttagatt gatttaaaac ttcattttta atttaaaagg atctaggtga agatcctttt |
| 6901 | tgataatctc atgaccaaaa tcccttaacg tgagttttcg ttccactgag cgtcagaccc |
| 6961 | cgtagaaaag atcaaaggat cttcttgaga tccttttttt ctgcgcgtaa tctgctgctt |
| 7021 | gcaaacaaaa aaaccaccgc taccagcggt ggtttgtttg ccggatcaag agctaccaac |
| 7081 | tctttttccg aaggtaactg gcttcagcag agcgcagata ccaaatactg tccttctagt |
| 7141 | gtagccgtag ttaggccacc acttcaagaa ctctgtagca ccgcctacat acctcgctct |
| 7201 | gctaatcctg ttaccagtgg ctgctgccag tggcgataag tcgtgtctta ccgggttgga |
| 7261 | ctcaagacga tagttaccgg ataaggcgca gcggtcgggc tgaacggggg gttcgtgcac |
| 7321 | acagcccagc ttggagcgaa cgacctacac cgaactgaga tacctacagc gtgagctatg |
| 7381 | agaaagcgcc acgcttcccg aagggagaaa ggcggacagg tatccggtaa gcggcagggt |
| 7441 | cggaacagga gagcgcacga gggagcttcc agggggaaac gcctggtatc tttatagtcc |
| 7501 | tgtcgggttt cgccacctct gacttgagcg tcgatttttg tgatgctcgt caggggggcg |
| 7561 | gagcctatgg aaaaacgcca gcaacgcggc ctttttacgg ttcctggcct tttgctggcc |
| 7621 | ttttgctcac atgttctttc ctgcgttatc ccctgattct gtggataacc gtattaccgc |
| 7681 | ctttgagtga gctgataccg ctcgccgcag ccgaacgacc gagcgcagcg agtcagtgag |
| 7741 | cgaggaagcg gaagagcgcc caatacgcaa accgcctctc cccgcgcgtt ggccgattca |
| 7801 | ttaatg |

SEQ ID NO: 59
Description: pAAV_CCR5.MND.II2ss.ADP.mAPRIL
cagctgcgcgctcgctcgctcactgaggccgcccgggcaaagcccgggcgtcgggcgacc
tttggtcgcccggcctcagtgagcgagcgagcgcgcagagagggagtggccaactccatc
actaggggttccttgtagttaatgattaacccgccatgctacttatctacgtagccatgc
tctagcggcctcggcctctgcataaataaaaaaaattagtcagccatgagcttggacgcg
tttggtgtggtggcgcctgtagtccccagccacttggaggggtgaggtgagaggattgct
tgagcccgggatggtccaggctgcagtgagccatgatcgtgccactgcactccagcctgg
gcgacagagtgagaccctgtctcacaacaacaacaacaacaacaaaaaggctgagctgca
ccatgcttgacccagtttcttaaaattgttgtcaaagcttcattcactccatggtgctat
agagcacaagattttatttggtgagatggtgctttcatgaattcccccaacagagccaag
ctctccatctagtggacagggaagctagcagcaaaccttcccttcactacaaaacttcat
tgcttggccaaaaagagagttaattcaatgtagacatctatgtaggcaattaaaaaccta
ttgatgtataaaacagtttgcattcatggagggcaactaaatacattctaggactttata
aaagatcactttttatttatgcacagggtggaacaagatggattatcaagtgtcaagtcc
aatctatgacatcaattattatacatcggagccctgccaaaaaatcaatgtgaagcaaat
cgcagcccgcctcctgcctccgctctactcactggtgttcatctttggttttgtgggcaa
catgctggtcatcctcatcctgataaactgcaaaaggctgaagagcatgactgacatcta
cctgctcaacctggccatctctgacctgtttttccttcttactgtccccttctgggctca
ctatgctgccgcccagtgggactttggaaatacaatgtgtcaacgaacagagaaacagga
gaatatgggccaaacaggatatctgtggtaagcagttcctgccccggctcagggccaaga
acagttggaacagcagaatatgggccaaacaggatatctgtggtaagcagttcctgcccc
ggctcagggccaagaacagatggtccccagatgcggtcccgccctcagcagtttctagag
aaccatcagatgtttccagggtgccccaaggacctgaaatgaccctgtgccttatttgaa
ctaaccaatcagttcgcttctcgcttctgttcgcgcgcttctgctccccgagctctatat
aagcagagctcgtttagtgaaccgtcagatcgcctggagacgccatccacgctgttttga
cttccatagaaggatctcgagatgtatcggatgcagctcttgagctgtatcgctctgtca
ctggcacttgttaccaactcagaggatgacgttaccaccacggaagaacttgcgcccgct
ttggtaccgcctccgaaaggaacctgtgccggttggatggctggaataccaggacatccc
ggacacaatggaacgccaggacgggatggacgcgacggcacgcccggagaaaaaggggag
aaaggggatgcaggcttgctcgggccaaagggcgaaaccggcgacgttggaatgacaggc
gctgaaggacctcggggttttccgggaaccccgggccgcaagggcgaacctggcgaggcc
gccgcagtattgacacagaaacagaaaaagcaacattccgtccttcatctggtccccatc
aacgcaacctccaaggatgatagtgatgtgaccgaggtaatgtggcaacccgcgcttagg
cgaggaagaggtctgcaggcgcagggatacggggtgcgaatccaagatgctggggtgtac
ctgctgtactcacaggttttgtttcaggacgtaacatttacgatggggcaggtcgtgtcc
cgagaaggacaagggagacaggaaacactcttccggtgtattagaagtatgccttcacat
cctgatcgcgcttacaactcttgttattccgctggcgtctttcacttgcatcagggcgac
atcctttcagtgataattccgagagcgcgggctaagttgaatcttagcccccacggcaca
tttctcggattcgtgaagctttgatgagtcgactgctttatttgtgaaatttgtgatgct
attgctttatttgtaaccattataagctgcaataaacaagttaacaacaacaattgcatt
cattttatgtttcaggttcagggggaggtgtgggaggttttttaaactctattttatagg
cttcttctctggaatcttcttcatcatcctcctgacaatcgataggtacctggctgtcgt
ccatgctgtgtttgctttaaaagccaggacggtcacctttggggtggtgacaagtgtgat
cacttgggtggtggctgtgtttgcgtctctcccaggaatcatctttaccagatctcaaaa
agaaggtcttcattacacctgcagctctcattttccatacagtcagtatcaattctggaa
gaatttccagacattaaagatagtcatcttggggctggtcctgccgctgcttgtcatggt
catctgctactcgggaatcctaaaaactctgcttcggtgtcgaaatgagaagaagaggca
cagggctgtgaggcttatcttcaccatcatgattgtttattttctcttctgggctcccta
caacattgtccttctcctgaacaccttccaggaattctttggcctgaataattgcagtag
ctctaacaggttggaccaagctatgcaggtgacagagactcttgggatgacgcactgctg
catcaaccccatcatctatgcctttgtcggggagaagttcagaaactacctcttagtctt
cttccaaaagcacattgccaaacgcttctgcaaatgctgttctattttccagcaagaggc
tcccgagcgagcaagctcagtttacacccgatccactggggagcaggaaatatctgtggg
cttgtgacacggactcaagtgggctggtgacccagtcagagttgtgcacatggcttagtt
ttcatacacaccgcggtctagagcatggctacgtagataagtagcatggcgggttaatca
ttaactacaaggaacccctagtgatggagttggccactccctctctgcgcgctcgctcgc
tcactgaggccgggcgaccaaaggtcgcccgacgcccgggctttgcccgggcggcctcag
tgagcgagcgagcgcgccagctggcgtaatagcgaagaggcccgcaccgatcgcccttcc
caacagttgcgcagcctgaatggcgaatggcgattccgttgcaatggctggcggtaatat
tgttctggatattaccagcaaggccgatagtttgagttcttctactcaggcaagtgatgt
tattactaatcaaagaagtattgcgacaacggttaatttgcgtgatggacagactctttt
actcggtggcctcactgattataaaaacacttctcaggattctggcgtaccgttcctgtc
taaaatccctttaatcggcctcctgtttagctcccgctctgattctaacgaggaaagcac
gttatacgtgctcgtcaaagcaaccatagtacgcgccctgtagcggcgcattaagcgcgg
cgggtgtggtggttacgcgcagcgtgaccgctacacttgccagcgccctagcgcccgctc
ctttcgctttcttcccttcctttctcgccacgttcgccggctttccccgtcaagctctaa
atcgggggctccctttagggttccgatttagtgctttacggcacctcgaccccaaaaaac
ttgattagggtgatggttcacgtagtgggccatcgccctgatagacggtttttcgccctt
tgacgttggagtccacgttctttaatagtggactcttgttccaaactggaacaacactca
accctatctcggtctattcttttgatttataagggattttgccgatttcggcctattggt
taaaaaatgagctgatttaacaaaaatttaacgcgaattttaacaaaatattaacgttta
caatttaaatatttgcttatacaatcttcctgtttttggggcttttctgattatcaaccg
gggtacatatgattgacatgctagttttacgattaccgttcatcgattctcttgtttgct
ccagactctcaggcaatgacctgatagcctttgtagagacctctcaaaaatagctaccct
ctccggcatgaatttatcagctagaacggttgaatatcatattgatggtgatttgactgt
ctccggcctttctcacccgtttgaatctttacctacacattactcaggcattgcatttaa
aatatatgagggttctaaaaatttttatccttgcgttgaaataaaggcttctcccgcaaa
agtattacagggtcataatgtttttggtacaaccgatttagctttatgctctgaggcttt
attgcttaattttgctaattctttgccttgcctgtatgatttattggatgttggaatcgc
ctgatgcggtattttctccttacgcatctgtgcggtatttcacaccgcatatggtgcact
ctcagtacaatctgctctgatgccgcatagttaagccagccccgacacccgccaacaccc
gctgacgcgccctgacgggcttgtctgctcccggcatccgcttacagacaagctgtgacc
gtctccgggagctgcatgtgtcagaggttttcaccgtcatcaccgaaacgcgcgagacga
aagggcctcgtgatacgcctatttttataggttaatgtcatgataataatggtttcttag
acgtcaggtggcacttttcggggaaatgtgcgcggaacccctatttgtttatttttctaa
atacattcaaatatgtatccgctcatgagacaataaccctgataaatgcttcaataatat
tgaaaaaggaagagtatgagtattcaacatttccgtgtcgcccttattcccttttttgcg
gcattttgccttcctgtttttgctcacccagaaacgctggtgaaagtaaaagatgctgaa
gatcagttgggtgcacgagtgggttacatcgaactggatctcaacagcggtaagatcctt
gagagttttcgccccgaagaacgttttccaatgatgagcacttttaaagttctgctatgt
ggcgcggtattatcccgtattgacgccgggcaagagcaactcggtcgccgcatacactat
tctcagaatgacttggttgagtactcaccagtcacagaaaagcatcttacggatggcatg
acagtaagagaattatgcagtgctgccataaccatgagtgataacactgcggccaactta
cttctgacaacgatcggaggaccgaaggagctaaccgcttttttgcacaacatgggggat
catgtaactcgccttgatcgttgggaaccggagctgaatgaagccataccaaacgacgag
cgtgacaccacgatgcctgtagcaatggcaacaacgttgcgcaaactattaactggcgaa
ctacttactctagcttcccggcaacaattaatagactggatggaggcggataaagttgca
ggaccacttctgcgctcggcccttccggctggctggtttattgctgataaatctggagcc
ggtgagcgtgggtctcgcggtatcattgcagcactggggccagatggtaagccctcccgt
atcgtagttatctacacgacggggagtcaggcaactatggatgaacgaaatagacagatc
gctgagataggtgcctcactgattaagcattggtaactgtcagaccaagtttactcatat
atactttagattgatttaaaacttcatttttaatttaaaaggatctaggtgaagatcctt
tttgataatctcatgaccaaaatcccttaacgtgagttttcgttccactgagcgtcagac
cccgtagaaaagatcaaaggatcttcttgagatcctttttttctgcgcgtaatctgctgc
ttgcaaacaaaaaaaccaccgctaccagcggtggtttgtttgccggatcaagagctacca
actctttttccgaaggtaactggcttcagcagagcgcagataccaaatactgtccttcta
gtgtagccgtagttaggccaccacttcaagaactctgtagcaccgcctacatacctcgct
ctgctaatcctgttaccagtggctgctgccagtggcgataagtcgtgtcttaccgggttg
gactcaagacgatagttaccggataaggcgcagcggtcgggctgaacggggggttcgtgc
acacagcccagcttggagcgaacgacctacaccgaactgagatacctacagcgtgagcta
tgagaaagcgccacgcttcccgaagggagaaaggcggacaggtatccggtaagcggcagg
gtcggaacaggagagcgcacgagggagcttccagggggaaacgcctggtatctttatagt
cctgtcgggtttcgccacctctgacttgagcgtcgatttttgtgatgctcgtcagggggg
cggagcctatggaaaaacgccagcaacgcggcctttttacggttcctggccttttgctgg
ccttttgctcacatgttctttcctgcgttatcccctgattctgtggataaccgtattacc
gcctttgagtgagctgataccgctcgccgcagccgaacgaccgagcgcagcgagtcagtg
agcgaggaagcggaagagcgcccaatacgcaaaccgcctctccccgcgcgttggccgatt
cattaatg
SEQ ID NO: 60
Description: pAAV CCR5.MND.IL6.Frun.T2A.GFP
cagctgcgcgctcgctcgctcactgaggccgcccgggcaaagcccgggcgtcgggcgacc
tttggtcgcccggcctcagtgagcgagcgagcgcgcagagagggagtggccaactccatc
actaggggttccttgtagttaatgattaacccgccatgctacttatctacgtagccatgc
tctagcggcctcggcctctgcataaataaaaaaaattagtcagccatgagcttggacgcg
tttggtgtggtggcgcctgtagtccccagccacttggaggggtgaggtgagaggattgct
tgagcccgggatggtccaggctgcagtgagccatgatcgtgccactgcactccagcctgg
gcgacagagtgagaccctgtctcacaacaacaacaacaacaacaaaaaggctgagctgca
ccatgcttgacccagtttcttaaaattgttgtcaaagcttcattcactccatggtgctat
agagcacaagattttatttggtgagatggtgctttcatgaattcccccaacagagccaag
ctctccatctagtggacagggaagctagcagcaaaccttcccttcactacaaaacttcat
tgcttggccaaaaagagagttaattcaatgtagacatctatgtaggcaattaaaaaccta
ttgatgtataaaacagtttgcattcatggagggcaactaaatacattctaggactttata
aaagatcactttttatttatgcacagggtggaacaagatggattatcaagtgtcaagtcc
aatctatgacatcaattattatacatcggagccctgccaaaaaatcaatgtgaagcaaat
cgcagcccgcctcctgcctccgctctactcactggtgttcatctttggttttgtgggcaa
catgctggtcatcctcatcctgataaactgcaaaaggctgaagagcatgactgacatcta
cctgctcaacctggccatctctgacctgtttttccttcttactgtccccttctgggctca
ctatgctgccgcccagtgggactttggaaatacaatgtgtcaacgaacagagaaacagga
gaatatgggccaaacaggatatctgtggtaagcagttcctgccccggctcagggccaaga
acagttggaacagcagaatatgggccaaacaggatatctgtggtaagcagttcctgcccc
ggctcagggccaagaacagatggtccccagatgcggtcccgccctcagcagtttctagag
aaccatcagatgtttccagggtgccccaaggacctgaaatgaccctgtgccttatttgaa
ctaaccaatcagttcgcttctcgcttctgttcgcgcgcttctgctccccgagctctatat
aagcagagctcgtttagtgaaccgtcagatcgcctggagacgccatccacgctgttttga
cttccatagaaggatctcgagatgaacagtttttctacttctgccttcggacccgtcgcc
tttagcctgggcctgctgctggtgctgcctgccgcattccccgctcctgtgccccctgga
gaagactccaaggatgtcgccgctcctcacagacagccactgactagctccgagaggatc
gacaaacagatccgctacattctggatggcattagcgccctgcgcaaggaaacctgcaac
aaatccaatatgtgtgagtctagtaaggaagcactggccgagaacaatctgaacctgccc
aagatggctgagaaagacggctgcttccagtctgggtttaatgaggaaacctgtctggtg
aaaatcattacagggctgctggagttcgaagtctacctggaatatctgcagaaccgattt
gagtcaagcgaggaacaggctcgggcagtgcagatgagcacaaaggtcctgatccagttc
ctgcagaagaaagccaaaaatctggacgctattaccacaccagatcccactaccaacgct
tctctgctgaccaagctgcaggcacagaatcagtggctgcaggatatgacaactcacctg
atcctgaggagtttcaaagaatttctgcagtcctctctgcgggcactgagacagatgcgc
gctaagcgaggatccggtgagggcagaggaagtcttctaacatgcggtgacgtggaggag
aatccgggccccatggtgagcaagggcgaggagctgttcaccggggtggtgcccatcctg
gtcgagctggacggcgacgtaaacggccacaagttcagcgtgtccggcgagggcgagggc
gatgccacctacggcaagctgaccctgaagttcatctgcaccaccggcaagctgcccgtg
ccctggcccaccctcgtgaccaccctgacctacggcgtgcagtgcttcagccgctacccc
gaccacatgaagcagcacgacttcttcaagtccgccatgcccgaaggctacgtccaggag
cgcaccatcttcttcaaggacgacggcaactacaagacccgcgccgaggtgaagttcgag
ggcgacaccctggtgaaccgcatcgagctgaagggcatcgacttcaaggaggacggcaac
atcctggggcacaagctggagtacaactacaacagccacaacgtctatatcatggccgac
aagcagaagaacggcatcaaggtgaacttcaagatccgccacaacatcgaggacggcagc
gtgcagctcgccgaccactaccagcagaacacccccatcggcgacggccccgtgctgctg
cccgacaaccactacctgagcacccagtccgccctgagcaaagaccccaacgagaagcgc
gatcacatggtcctgctggagttcgtgaccgccgccgggatcactctcggcatggacgag
ctgtacaagtgaatctagagtcgactgctttatttgtgaaatttgtgatgctattgcttt
atttgtaaccattataagctgcaataaacaagttaacaacaacaattgcattcattttat
gtttcaggttcagggggaggtgtgggaggttttttaaactctattttataggcttcttct
ctggaatcttcttcatcatcctcctgacaatcgataggtacctggctgtcgtccatgctg
tgtttgctttaaaagccaggacggtcacctttggggtggtgacaagtgtgatcacttggg
tggtggctgtgtttgcgtctctcccaggaatcatctttaccagatctcaaaaagaaggtc
ttcattacacctgcagctctcattttccatacagtcagtatcaattctggaagaatttcc
agacattaaagatagtcatcttggggctggtcctgccgctgcttgtcatggtcatctgct
actcgggaatcctaaaaactctgcttcggtgtcgaaatgagaagaagaggcacagggctg
tgaggcttatcttcaccatcatgattgtttattttctcttctgggctccctacaacattg
tccttctcctgaacaccttccaggaattctttggcctgaataattgcagtagctctaaca
ggttggaccaagctatgcaggtgacagagactcttgggatgacgcactgctgcatcaacc
ccatcatctatgcctttgtcggggagaagttcagaaactacctcttagtcttcttccaaa
agcacattgccaaacgcttctgcaaatgctgttctattttccagcaagaggctcccgagc
gagcaagctcagtttacacccgatccactggggagcaggaaatatctgtgggcttgtgac
acggactcaagtgggctggtgacccagtcagagttgtgcacatggcttagttttcataca
caccgcggtctagagcatggctacgtagataagtagcatggcgggttaatcattaactac
aaggaacccctagtgatggagttggccactccctctctgcgcgctcgctcgctcactgag
gccgggcgaccaaaggtcgcccgacgcccgggctttgcccgggcggcctcagtgagcgag
cgagcgcgccagctggcgtaatagcgaagaggcccgcaccgatcgcccttcccaacagtt
gcgcagcctgaatggcgaatggcgattccgttgcaatggctggcggtaatattgttctgg
atattaccagcaaggccgatagtttgagttcttctactcaggcaagtgatgttattacta
atcaaagaagtattgcgacaacggttaatttgcgtgatggacagactcttttactcggtg
gcctcactgattataaaaacacttctcaggattctggcgtaccgttcctgtctaaaatcc
ctttaatcggcctcctgtttagctcccgctctgattctaacgaggaaagcacgttatacg
tgctcgtcaaagcaaccatagtacgcgccctgtagcggcgcattaagcgcggcgggtgtg
gtggttacgcgcagcgtgaccgctacacttgccagcgccctagcgcccgctcctttcgct
ttcttcccttcctttctcgccacgttcgccggctttccccgtcaagctctaaatcggggg
ctccctttagggttccgatttagtgctttacggcacctcgaccccaaaaaacttgattag
ggtgatggttcacgtagtgggccatcgccctgatagacggtttttcgccctttgacgttg
gagtccacgttctttaatagtggactcttgttccaaactggaacaacactcaaccctatc
tcggtctattcttttgatttataagggattttgccgatttcggcctattggttaaaaaat
gagctgatttaacaaaaatttaacgcgaattttaacaaaatattaacgtttacaatttaa
atatttgcttatacaatcttcctgtttttggggcttttctgattatcaaccggggtacat
atgattgacatgctagttttacgattaccgttcatcgattctcttgtttgctccagactc
tcaggcaatgacctgatagcctttgtagagacctctcaaaaatagctaccctctccggca
tgaatttatcagctagaacggttgaatatcatattgatggtgatttgactgtctccggcc
tttctcacccgtttgaatctttacctacacattactcaggcattgcatttaaaatatatg
agggttctaaaaatttttatccttgcgttgaaataaaggcttctcccgcaaaagtattac
agggtcataatgtttttggtacaaccgatttagctttatgctctgaggctttattgctta
attttgctaattctttgccttgcctgtatgatttattggatgttggaatcgcctgatgcg
gtattttctccttacgcatctgtgcggtatttcacaccgcatatggtgcactctcagtac
aatctgctctgatgccgcatagttaagccagccccgacacccgccaacacccgctgacgc
gccctgacgggcttgtctgctcccggcatccgcttacagacaagctgtgaccgtctccgg
gagctgcatgtgtcagaggttttcaccgtcatcaccgaaacgcgcgagacgaaagggcct
cgtgatacgcctatttttataggttaatgtcatgataataatggtttcttagacgtcagg
tggcacttttcggggaaatgtgcgcggaacccctatttgtttatttttctaaatacattc
aaatatgtatccgctcatgagacaataaccctgataaatgcttcaataatattgaaaaag
gaagagtatgagtattcaacatttccgtgtcgcccttattcccttttttgcggcattttg
ccttcctgtttttgctcacccagaaacgctggtgaaagtaaaagatgctgaagatcagtt
gggtgcacgagtgggttacatcgaactggatctcaacagcggtaagatccttgagagttt
tcgccccgaagaacgttttccaatgatgagcacttttaaagttctgctatgtggcgcggt
attatcccgtattgacgccgggcaagagcaactcggtcgccgcatacactattctcagaa
tgacttggttgagtactcaccagtcacagaaaagcatcttacggatggcatgacagtaag
agaattatgcagtgctgccataaccatgagtgataacactgcggccaacttacttctgac
aacgatcggaggaccgaaggagctaaccgcttttttgcacaacatgggggatcatgtaac
tcgccttgatcgttgggaaccggagctgaatgaagccataccaaacgacgagcgtgacac
cacgatgcctgtagcaatggcaacaacgttgcgcaaactattaactggcgaactacttac
tctagcttcccggcaacaattaatagactggatggaggcggataaagttgcaggaccact
tctgcgctcggcccttccggctggctggtttattgctgataaatctggagccggtgagcg
tgggtctcgcggtatcattgcagcactggggccagatggtaagccctcccgtatcgtagt
tatctacacgacggggagtcaggcaactatggatgaacgaaatagacagatcgctgagat
aggtgcctcactgattaagcattggtaactgtcagaccaagtttactcatatatacttta
gattgatttaaaacttcatttttaatttaaaaggatctaggtgaagatcctttttgataa
tctcatgaccaaaatcccttaacgtgagttttcgttccactgagcgtcagaccccgtaga
aaagatcaaaggatcttcttgagatcctttttttctgcgcgtaatctgctgcttgcaaac
aaaaaaaccaccgctaccagcggtggtttgtttgccggatcaagagctaccaactctttt
tccgaaggtaactggcttcagcagagcgcagataccaaatactgtccttctagtgtagcc
gtagttaggccaccacttcaagaactctgtagcaccgcctacatacctcgctctgctaat
cctgttaccagtggctgctgccagtggcgataagtcgtgtcttaccgggttggactcaag
acgatagttaccggataaggcgcagcggtcgggctgaacggggggttcgtgcacacagcc
cagcttggagcgaacgacctacaccgaactgagatacctacagcgtgagctatgagaaag
cgccacgcttcccgaagggagaaaggcggacaggtatccggtaagcggcagggtcggaac
aggagagcgcacgagggagcttccagggggaaacgcctggtatctttatagtcctgtcgg
gtttcgccacctctgacttgagcgtcgatttttgtgatgctcgtcaggggggcggagcct
atggaaaaacgccagcaacgcggcctttttacggttcctggccttttgctggccttttgc
tcacatgttctttcctgcgttatcccctgattctgtggataaccgtattaccgcctttga
gtgagctgataccgctcgccgcagccgaacgaccgagcgcagcgagtcagtgagcgagga
agcggaagagcgcccaatacgcaaaccgcctctccccgcgcgttggccgattcattaatg
SEQ ID NO: 61
Description: pAAV CCR5.MND.II2ss-ADP.mAPRIL.Furin.T2A.GFP
cagctgcgcgctcgctcgctcactgaggccgcccgggcaaagcccgggcgtcgggcgacc
tttggtcgcccggcctcagtgagcgagcgagcgcgcagagagggagtggccaactccatc
actaggggttccttgtagttaatgattaacccgccatgctacttatctacgtagccatgc
tctagcggcctcggcctctgcataaataaaaaaaattagtcagccatgagcttggacgcg
tttggtgtggtggcgcctgtagtccccagccacttggaggggtgaggtgagaggattgct
tgagcccgggatggtccaggctgcagtgagccatgatcgtgccactgcactccagcctgg
gcgacagagtgagaccctgtctcacaacaacaacaacaacaacaaaaaggctgagctgca
ccatgcttgacccagtttcttaaaattgttgtcaaagcttcattcactccatggtgctat
agagcacaagattttatttggtgagatggtgctttcatgaattcccccaacagagccaag
ctctccatctagtggacagggaagctagcagcaaaccttcccttcactacaaaacttcat
tgcttggccaaaaagagagttaattcaatgtagacatctatgtaggcaattaaaaaccta
ttgatgtataaaacagtttgcattcatggagggcaactaaatacattctaggactttata
aaagatcactttttatttatgcacagggtggaacaagatggattatcaagtgtcaagtcc
aatctatgacatcaattattatacatcggagccctgccaaaaaatcaatgtgaagcaaat
cgcagcccgcctcctgcctccgctctactcactggtgttcatctttggttttgtgggcaa
catgctggtcatcctcatcctgataaactgcaaaaggctgaagagcatgactgacatcta
cctgctcaacctggccatctctgacctgtttttccttcttactgtccccttctgggctca
ctatgctgccgcccagtgggactttggaaatacaatgtgtcaacgaacagagaaacagga
gaatatgggccaaacaggatatctgtggtaagcagttcctgccccggctcagggccaaga
acagttggaacagcagaatatgggccaaacaggatatctgtggtaagcagttcctgcccc
ggctcagggccaagaacagatggtccccagatgcggtcccgccctcagcagtttctagag
aaccatcagatgtttccagggtgccccaaggacctgaaatgaccctgtgccttatttgaa
ctaaccaatcagttcgcttctcgcttctgttcgcgcgcttctgctccccgagctctatat
aagcagagctcgtttagtgaaccgtcagatcgcctggagacgccatccacgctgttttga
cttccatagaaggatctcgagatgtatcggatgcagctcttgagctgtatcgctctgtca
ctggcacttgttaccaactcagaggatgacgttaccaccacggaagaacttgcgcccgct
ttggtaccgcctccgaaaggaacctgtgccggttggatggctggaataccaggacatccc
ggacacaatggaacgccaggacgggatggacgcgacggcacgcccggagaaaaaggggag
aaaggggatgcaggcttgctcgggccaaagggcgaaaccggcgacgttggaatgacaggc
gctgaaggacctcggggttttccgggaaccccgggccgcaagggcgaacctggcgaggcc
gccgcagtattgacacagaaacagaaaaagcaacattccgtccttcatctggtccccatc
aacgcaacctccaaggatgatagtgatgtgaccgaggtaatgtggcaacccgcgcttagg
cgaggaagaggtctgcaggcgcagggatacggggtgcgaatccaagatgctggggtgtac
ctgctgtactcacaggttttgtttcaggacgtaacatttacgatggggcaggtcgtgtcc
cgagaaggacaagggagacaggaaacactcttccggtgtattagaagtatgccttcacat
cctgatcgcgcttacaactcttgttattccgctggcgtctttcacttgcatcagggcgac
atcctttcagtgataattccgagagcgcgggctaagttgaatcttagcccccacggcaca
tttctcggattcgtgaagcttcgcgctaagcgaggatccggtgagggcagaggaagtctt
ctaacatgcggtgacgtggaggagaatccgggccccatggtgagcaagggcgaggagctg
ttcaccggggtggtgcccatcctggtcgagctggacggcgacgtaaacggccacaagttc
agcgtgtccggcgagggcgagggcgatgccacctacggcaagctgaccctgaagttcatc
tgcaccaccggcaagctgcccgtgccctggcccaccctcgtgaccaccctgacctacggc
gtgcagtgcttcagccgctaccccgaccacatgaagcagcacgacttcttcaagtccgcc
atgcccgaaggctacgtccaggagcgcaccatcttcttcaaggacgacggcaactacaag
acccgcgccgaggtgaagttcgagggcgacaccctggtgaaccgcatcgagctgaagggc
atcgacttcaaggaggacggcaacatcctggggcacaagctggagtacaactacaacagc
cacaacgtctatatcatggccgacaagcagaagaacggcatcaaggtgaacttcaagatc
cgccacaacatcgaggacggcagcgtgcagctcgccgaccactaccagcagaacaccccc
atcggcgacggccccgtgctgctgcccgacaaccactacctgagcacccagtccgccctg
agcaaagaccccaacgagaagcgcgatcacatggtcctgctggagttcgtgaccgccgcc
gggatcactctcggcatggacgagctgtacaagtgaatctagagtcgactgctttatttg
tgaaatttgtgatgctattgctttatttgtaaccattataagctgcaataaacaagttaa
caacaacaattgcattcattttatgtttcaggttcagggggaggtgtgggaggtttttta
aactctattttataggcttcttctctggaatcttcttcatcatcctcctgacaatcgata
ggtacctggctgtcgtccatgctgtgtttgctttaaaagccaggacggtcacctttgggg
tggtgacaagtgtgatcacttgggtggtggctgtgtttgcgtctctcccaggaatcatct
ttaccagatctcaaaaagaaggtcttcattacacctgcagctctcattttccatacagtc
agtatcaattctggaagaatttccagacattaaagatagtcatcttggggctggtcctgc
cgctgcttgtcatggtcatctgctactcgggaatcctaaaaactctgcttcggtgtcgaa
atgagaagaagaggcacagggctgtgaggcttatcttcaccatcatgattgtttattttc
tcttctgggctccctacaacattgtccttctcctgaacaccttccaggaattctttggcc
tgaataattgcagtagctctaacaggttggaccaagctatgcaggtgacagagactcttg
ggatgacgcactgctgcatcaaccccatcatctatgcctttgtcggggagaagttcagaa
actacctcttagtcttcttccaaaagcacattgccaaacgcttctgcaaatgctgttcta
ttttccagcaagaggctcccgagcgagcaagctcagtttacacccgatccactggggagc
aggaaatatctgtgggcttgtgacacggactcaagtgggctggtgacccagtcagagttg
tgcacatggcttagttttcatacacaccgcggtctagagcatggctacgtagataagtag
catggcgggttaatcattaactacaaggaacccctagtgatggagttggccactccctct
ctgcgcgctcgctcgctcactgaggccgggcgaccaaaggtcgcccgacgcccgggcttt
gcccgggcggcctcagtgagcgagcgagcgcgccagctggcgtaatagcgaagaggcccg
caccgatcgcccttcccaacagttgcgcagcctgaatggcgaatggcgattccgttgcaa
tggctggcggtaatattgttctggatattaccagcaaggccgatagtttgagttcttcta
ctcaggcaagtgatgttattactaatcaaagaagtattgcgacaacggttaatttgcgtg
atggacagactcttttactcggtggcctcactgattataaaaacacttctcaggattctg
gcgtaccgttcctgtctaaaatccctttaatcggcctcctgtttagctcccgctctgatt
ctaacgaggaaagcacgttatacgtgctcgtcaaagcaaccatagtacgcgccctgtagc
ggcgcattaagcgcggcgggtgtggtggttacgcgcagcgtgaccgctacacttgccagc
gccctagcgcccgctcctttcgctttcttcccttcctttctcgccacgttcgccggcttt
ccccgtcaagctctaaatcgggggctccctttagggttccgatttagtgctttacggcac
ctcgaccccaaaaaacttgattagggtgatggttcacgtagtgggccatcgccctgatag
acggtttttcgccctttgacgttggagtccacgttctttaatagtggactcttgttccaa
actggaacaacactcaaccctatctcggtctattcttttgatttataagggattttgccg
atttcggcctattggttaaaaaatgagctgatttaacaaaaatttaacgcgaattttaac
aaaatattaacgtttacaatttaaatatttgcttatacaatcttcctgtttttggggctt
ttctgattatcaaccggggtacatatgattgacatgctagttttacgattaccgttcatc
gattctcttgtttgctccagactctcaggcaatgacctgatagcctttgtagagacctct
caaaaatagctaccctctccggcatgaatttatcagctagaacggttgaatatcatattg
atggtgatttgactgtctccggcctttctcacccgtttgaatctttacctacacattact
caggcattgcatttaaaatatatgagggttctaaaaatttttatccttgcgttgaaataa
aggcttctcccgcaaaagtattacagggtcataatgtttttggtacaaccgatttagctt
tatgctctgaggctttattgcttaattttgctaattctttgccttgcctgtatgatttat
tggatgttggaatcgcctgatgcggtattttctccttacgcatctgtgcggtatttcaca
ccgcatatggtgcactctcagtacaatctgctctgatgccgcatagttaagccagccccg
acacccgccaacacccgctgacgcgccctgacgggcttgtctgctcccggcatccgctta
cagacaagctgtgaccgtctccgggagctgcatgtgtcagaggttttcaccgtcatcacc
gaaacgcgcgagacgaaagggcctcgtgatacgcctatttttataggttaatgtcatgat
aataatggtttcttagacgtcaggtggcacttttcggggaaatgtgcgcggaacccctat
ttgtttatttttctaaatacattcaaatatgtatccgctcatgagacaataaccctgata
aatgcttcaataatattgaaaaaggaagagtatgagtattcaacatttccgtgtcgccct
tattcccttttttgcggcattttgccttcctgtttttgctcacccagaaacgctggtgaa
agtaaaagatgctgaagatcagttgggtgcacgagtgggttacatcgaactggatctcaa
cagcggtaagatccttgagagttttcgccccgaagaacgttttccaatgatgagcacttt
taaagttctgctatgtggcgcggtattatcccgtattgacgccgggcaagagcaactcgg
tcgccgcatacactattctcagaatgacttggttgagtactcaccagtcacagaaaagca
tcttacggatggcatgacagtaagagaattatgcagtgctgccataaccatgagtgataa
cactgcggccaacttacttctgacaacgatcggaggaccgaaggagctaaccgctttttt
gcacaacatgggggatcatgtaactcgccttgatcgttgggaaccggagctgaatgaagc
cataccaaacgacgagcgtgacaccacgatgcctgtagcaatggcaacaacgttgcgcaa
actattaactggcgaactacttactctagcttcccggcaacaattaatagactggatgga
ggcggataaagttgcaggaccacttctgcgctcggcccttccggctggctggtttattgc
tgataaatctggagccggtgagcgtgggtctcgcggtatcattgcagcactggggccaga
tggtaagccctcccgtatcgtagttatctacacgacggggagtcaggcaactatggatga
acgaaatagacagatcgctgagataggtgcctcactgattaagcattggtaactgtcaga
ccaagtttactcatatatactttagattgatttaaaacttcatttttaatttaaaaggat
ctaggtgaagatcctttttgataatctcatgaccaaaatcccttaacgtgagttttcgtt
ccactgagcgtcagaccccgtagaaaagatcaaaggatcttcttgagatcctttttttct
gcgcgtaatctgctgcttgcaaacaaaaaaaccaccgctaccagcggtggtttgtttgcc
ggatcaagagctaccaactctttttccgaaggtaactggcttcagcagagcgcagatacc
aaatactgtccttctagtgtagccgtagttaggccaccacttcaagaactctgtagcacc
gcctacatacctcgctctgctaatcctgttaccagtggctgctgccagtggcgataagtc
gtgtcttaccgggttggactcaagacgatagttaccggataaggcgcagcggtcgggctg
aacggggggttcgtgcacacagcccagcttggagcgaacgacctacaccgaactgagata
cctacagcgtgagctatgagaaagcgccacgcttcccgaagggagaaaggcggacaggta
tccggtaagcggcagggtcggaacaggagagcgcacgagggagcttccagggggaaacgc
ctggtatctttatagtcctgtcgggtttcgccacctctgacttgagcgtcgatttttgtg
atgctcgtcaggggggcggagcctatggaaaaacgccagcaacgcggcctttttacggtt
cctggccttttgctggccttttgctcacatgttctttcctgcgttatcccctgattctgt
ggataaccgtattaccgcctttgagtgagctgataccgctcgccgcagccgaacgaccga
gcgcagcgagtcagtgagcgaggaagcggaagagcgcccaatacgcaaaccgcctctccc
cgcgcgttggccgattcattaatg
SEQ ID NO: 62
Description: pAAV CCR5 MND IL6
cagctgcgcgctcgctcgctcactgaggccgcccgggcaaagcccgggcgtcgggcgacc
tttggtcgcccggcctcagtgagcgagcgagcgcgcagagagggagtggccaactccatc
actaggggttccttgtagttaatgattaacccgccatgctacttatctacgtagccatgc
tctagcggcctcggcctctgcataaataaaaaaaattagtcagccatgagcttggacgcg
tttggtgtggtggcgcctgtagtccccagccacttggaggggtgaggtgagaggattgct
tgagcccgggatggtccaggctgcagtgagccatgatcgtgccactgcactccagcctgg
gcgacagagtgagaccctgtctcacaacaacaacaacaacaacaaaaaggctgagctgca
ccatgcttgacccagtttcttaaaattgttgtcaaagcttcattcactccatggtgctat
agagcacaagattttatttggtgagatggtgctttcatgaattcccccaacagagccaag
ctctccatctagtggacagggaagctagcagcaaaccttcccttcactacaaaacttcat
tgcttggccaaaaagagagttaattcaatgtagacatctatgtaggcaattaaaaaccta
ttgatgtataaaacagtttgcattcatggagggcaactaaatacattctaggactttata
aaagatcactttttatttatgcacagggtggaacaagatggattatcaagtgtcaagtcc
aatctatgacatcaattattatacatcggagccctgccaaaaaatcaatgtgaagcaaat
cgcagcccgcctcctgcctccgctctactcactggtgttcatctttggttttgtgggcaa
catgctggtcatcctcatcctgataaactgcaaaaggctgaagagcatgactgacatcta
cctgctcaacctggccatctctgacctgtttttccttcttactgtccccttctgggctca
ctatgctgccgcccagtgggactttggaaatacaatgtgtcaacgaacagagaaacagga
gaatatgggccaaacaggatatctgtggtaagcagttcctgccccggctcagggccaaga
acagttggaacagcagaatatgggccaaacaggatatctgtggtaagcagttcctgcccc
ggctcagggccaagaacagatggtccccagatgcggtcccgccctcagcagtttctagag
aaccatcagatgtttccagggtgccccaaggacctgaaatgaccctgtgccttatttgaa
ctaaccaatcagttcgcttctcgcttctgttcgcgcgcttctgctccccgagctctatat
aagcagagctcgtttagtgaaccgtcagatcgcctggagacgccatccacgctgttttga
cttccatagaaggatctcgagatgaacagtttttctacttctgccttcggacccgtcgcc
tttagcctgggcctgctgctggtgctgcctgccgcattccccgctcctgtgccccctgga
gaagactccaaggatgtcgccgctcctcacagacagccactgactagctccgagaggatc
gacaaacagatccgctacattctggatggcattagcgccctgcgcaaggaaacctgcaac
aaatccaatatgtgtgagtctagtaaggaagcactggccgagaacaatctgaacctgccc
aagatggctgagaaagacggctgcttccagtctgggtttaatgaggaaacctgtctggtg
aaaatcattacagggctgctggagttcgaagtctacctggaatatctgcagaaccgattt
gagtcaagcgaggaacaggctcgggcagtgcagatgagcacaaaggtcctgatccagttc
ctgcagaagaaagccaaaaatctggacgctattaccacaccagatcccactaccaacgct
tctctgctgaccaagctgcaggcacagaatcagtggctgcaggatatgacaactcacctg
atcctgaggagtttcaaagaatttctgcagtcctctctgcgggcactgagacagatgtga
gtcgactgctttatttgtgaaatttgtgatgctattgctttatttgtaaccattataagc
tgcaataaacaagttaacaacaacaattgcattcattttatgtttcaggttcagggggag
gtgtgggaggttttttaaactctattttataggcttcttctctggaatcttcttcatcat
cctcctgacaatcgataggtacctggctgtcgtccatgctgtgtttgctttaaaagccag
gacggtcacctttggggtggtgacaagtgtgatcacttgggtggtggctgtgtttgcgtc
tctcccaggaatcatctttaccagatctcaaaaagaaggtcttcattacacctgcagctc
tcattttccatacagtcagtatcaattctggaagaatttccagacattaaagatagtcat
cttggggctggtcctgccgctgcttgtcatggtcatctgctactcgggaatcctaaaaac
tctgcttcggtgtcgaaatgagaagaagaggcacagggctgtgaggcttatcttcaccat
catgattgtttattttctcttctgggctccctacaacattgtccttctcctgaacacctt
ccaggaattctttggcctgaataattgcagtagctctaacaggttggaccaagctatgca
ggtgacagagactcttgggatgacgcactgctgcatcaaccccatcatctatgcctttgt
cggggagaagttcagaaactacctcttagtcttcttccaaaagcacattgccaaacgctt
ctgcaaatgctgttctattttccagcaagaggctcccgagcgagcaagctcagtttacac
ccgatccactggggagcaggaaatatctgtgggcttgtgacacggactcaagtgggctgg
tgacccagtcagagttgtgcacatggcttagttttcatacacaccgcggtctagagcatg
gctacgtagataagtagcatggcgggttaatcattaactacaaggaacccctagtgatgg
agttggccactccctctctgcgcgctcgctcgctcactgaggccgggcgaccaaaggtcg
cccgacgcccgggctttgcccgggcggcctcagtgagcgagcgagcgcgccagctggcgt
aatagcgaagaggcccgcaccgatcgcccttcccaacagttgcgcagcctgaatggcgaa
tggcgattccgttgcaatggctggcggtaatattgttctggatattaccagcaaggccga
tagtttgagttcttctactcaggcaagtgatgttattactaatcaaagaagtattgcgac
aacggttaatttgcgtgatggacagactcttttactcggtggcctcactgattataaaaa
cacttctcaggattctggcgtaccgttcctgtctaaaatccctttaatcggcctcctgtt
tagctcccgctctgattctaacgaggaaagcacgttatacgtgctcgtcaaagcaaccat
agtacgcgccctgtagcggcgcattaagcgcggcgggtgtggtggttacgcgcagcgtga
ccgctacacttgccagcgccctagcgcccgctcctttcgctttcttcccttcctttctcg
ccacgttcgccggctttccccgtcaagctctaaatcgggggctccctttagggttccgat
ttagtgctttacggcacctcgaccccaaaaaacttgattagggtgatggttcacgtagtg
ggccatcgccctgatagacggtttttcgccctttgacgttggagtccacgttctttaata
gtggactcttgttccaaactggaacaacactcaaccctatctcggtctattcttttgatt
tataagggattttgccgatttcggcctattggttaaaaaatgagctgatttaacaaaaat
ttaacgcgaattttaacaaaatattaacgtttacaatttaaatatttgcttatacaatct
tcctgtttttggggcttttctgattatcaaccggggtacatatgattgacatgctagttt
tacgattaccgttcatcgattctcttgtttgctccagactctcaggcaatgacctgatag
cctttgtagagacctctcaaaaatagctaccctctccggcatgaatttatcagctagaac
ggttgaatatcatattgatggtgatttgactgtctccggcctttctcacccgtttgaatc
tttacctacacattactcaggcattgcatttaaaatatatgagggttctaaaaattttta
tccttgcgttgaaataaaggcttctcccgcaaaagtattacagggtcataatgtttttgg
tacaaccgatttagctttatgctctgaggctttattgcttaattttgctaattctttgcc
ttgcctgtatgatttattggatgttggaatcgcctgatgcggtattttctccttacgcat
ctgtgcggtatttcacaccgcatatggtgcactctcagtacaatctgctctgatgccgca
tagttaagccagccccgacacccgccaacacccgctgacgcgccctgacgggcttgtctg
ctcccggcatccgcttacagacaagctgtgaccgtctccgggagctgcatgtgtcagagg
ttttcaccgtcatcaccgaaacgcgcgagacgaaagggcctcgtgatacgcctattttta
taggttaatgtcatgataataatggtttcttagacgtcaggtggcacttttcggggaaat
gtgcgcggaacccctatttgtttatttttctaaatacattcaaatatgtatccgctcatg
agacaataaccctgataaatgcttcaataatattgaaaaaggaagagtatgagtattcaa
catttccgtgtcgcccttattcccttttttgcggcattttgccttcctgtttttgctcac
ccagaaacgctggtgaaagtaaaagatgctgaagatcagttgggtgcacgagtgggttac
atcgaactggatctcaacagcggtaagatccttgagagttttcgccccgaagaacgtttt
ccaatgatgagcacttttaaagttctgctatgtggcgcggtattatcccgtattgacgcc
gggcaagagcaactcggtcgccgcatacactattctcagaatgacttggttgagtactca
ccagtcacagaaaagcatcttacggatggcatgacagtaagagaattatgcagtgctgcc
ataaccatgagtgataacactgcggccaacttacttctgacaacgatcggaggaccgaag
gagctaaccgcttttttgcacaacatgggggatcatgtaactcgccttgatcgttgggaa
ccggagctgaatgaagccataccaaacgacgagcgtgacaccacgatgcctgtagcaatg
gcaacaacgttgcgcaaactattaactggcgaactacttactctagcttcccggcaacaa
ttaatagactggatggaggcggataaagttgcaggaccacttctgcgctcggcccttccg
gctggctggtttattgctgataaatctggagccggtgagcgtgggtctcgcggtatcatt
gcagcactggggccagatggtaagccctcccgtatcgtagttatctacacgacggggagt
caggcaactatggatgaacgaaatagacagatcgctgagataggtgcctcactgattaag
cattggtaactgtcagaccaagtttactcatatatactttagattgatttaaaacttcat
ttttaatttaaaaggatctaggtgaagatcctttttgataatctcatgaccaaaatccct
taacgtgagttttcgttccactgagcgtcagaccccgtagaaaagatcaaaggatcttct
tgagatcctttttttctgcgcgtaatctgctgcttgcaaacaaaaaaaccaccgctacca
gcggtggtttgtttgccggatcaagagctaccaactctttttccgaaggtaactggcttc
agcagagcgcagataccaaatactgtccttctagtgtagccgtagttaggccaccacttc
aagaactctgtagcaccgcctacatacctcgctctgctaatcctgttaccagtggctgct
gccagtggcgataagtcgtgtcttaccgggttggactcaagacgatagttaccggataag
gcgcagcggtcgggctgaacggggggttcgtgcacacagcccagcttggagcgaacgacc
tacaccgaactgagatacctacagcgtgagctatgagaaagcgccacgcttcccgaaggg
agaaaggcggacaggtatccggtaagcggcagggtcggaacaggagagcgcacgagggag
cttccagggggaaacgcctggtatctttatagtcctgtcgggtttcgccacctctgactt
gagcgtcgatttttgtgatgctcgtcaggggggcggagcctatggaaaaacgccagcaac
gcggcctttttacggttcctggccttttgctggccttttgctcacatgttctttcctgcg
ttatcccctgattctgtggataaccgtattaccgcctttgagtgagctgataccgctcgc
cgcagccgaacgaccgagcgcagcgagtcagtgagcgaggaagcggaagagcgcccaata
cgcaaaccgcctctccccgcgcgttggccgattcattaatg

Alternative 4: Testing Primary B Cell Transduction with AAV

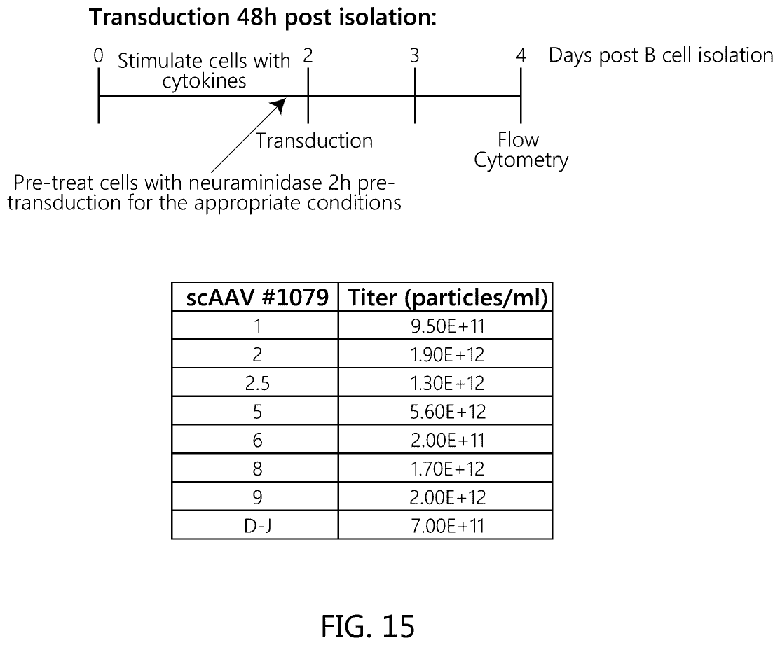

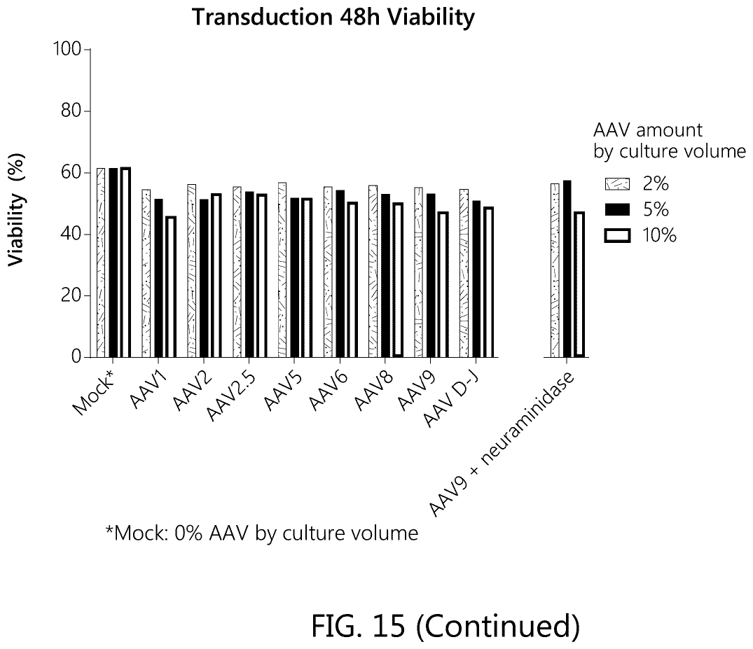

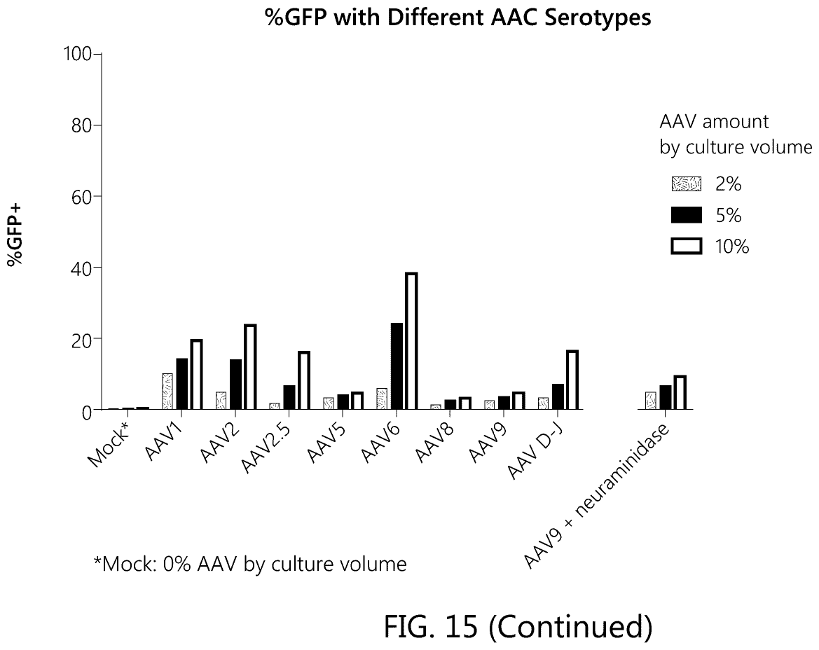

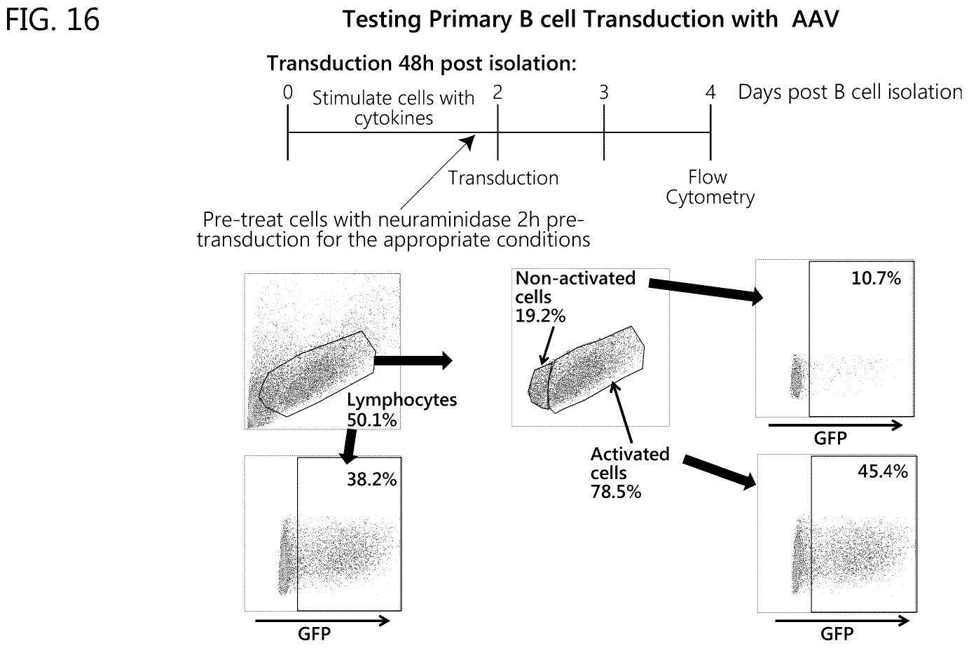

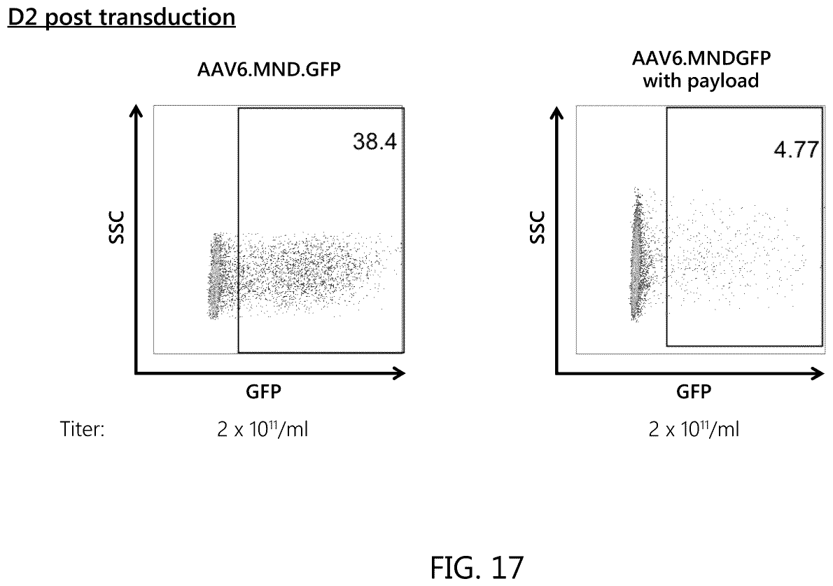

Collection of cells and transduction of the B cells with AAV are described in the section entitled “Primary human CD19+ cell gene editing” of this paper. Cells were also pretreated with neuraminidase at least two hours prior to transduction for the appropriate conditions. As shown in FIG. 15, the AAV amount transduced into the cells varied by culture volume. As shown in the bar graphs the AAV is in order on the x axis as the AAV amount by culture volume at 2%, 5% and 10% consecutively. As shown, the percent GFP expressed with different AAV serotypes was increased using serotype AAV6. As shown in FIG. 16, AAV6 transduction in primary B cells is more effective in activated B cells. As expected, larger AAV6 vectors (AAV6.MNDGFP with payload) exhibit lower transduction rates than smaller AAV6 vectors (FIG. 17).

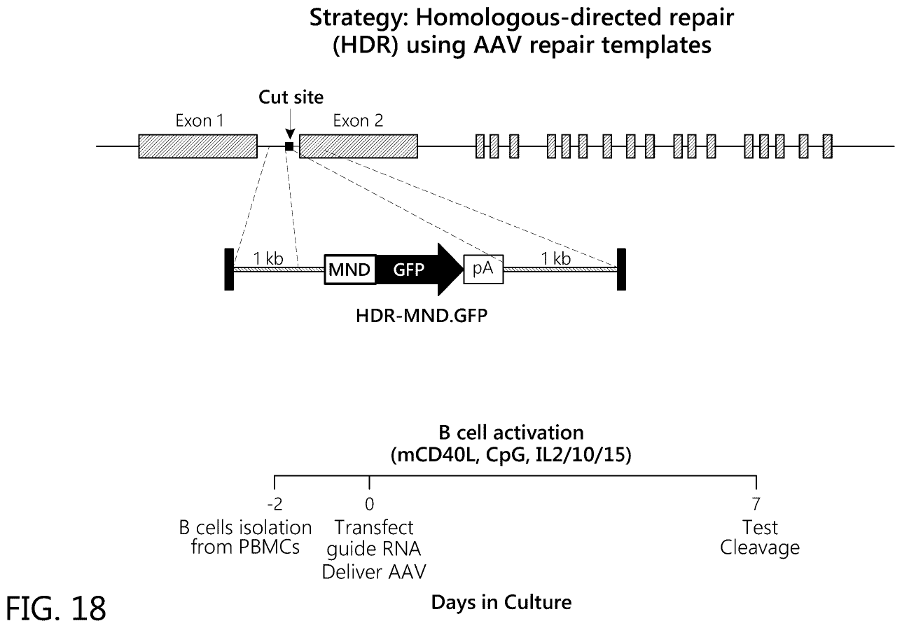

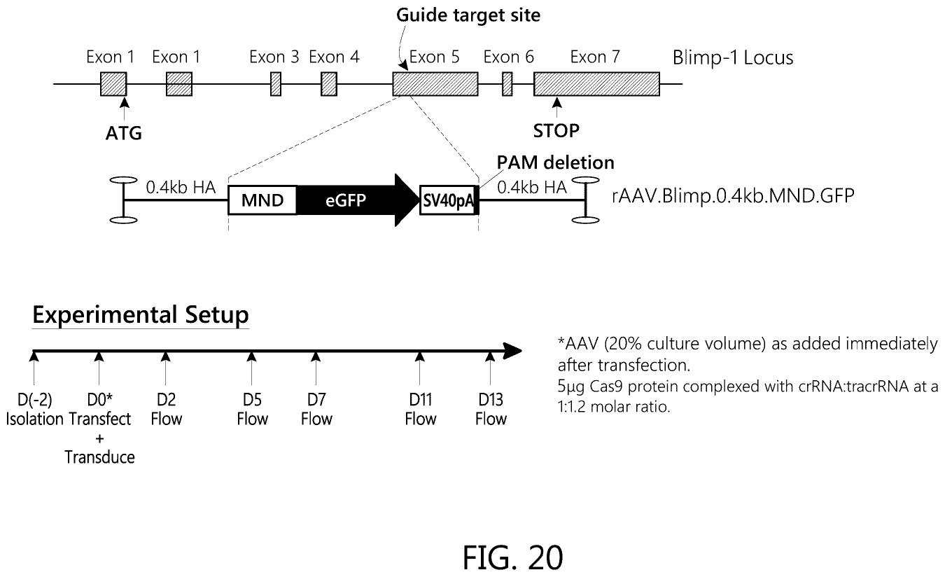

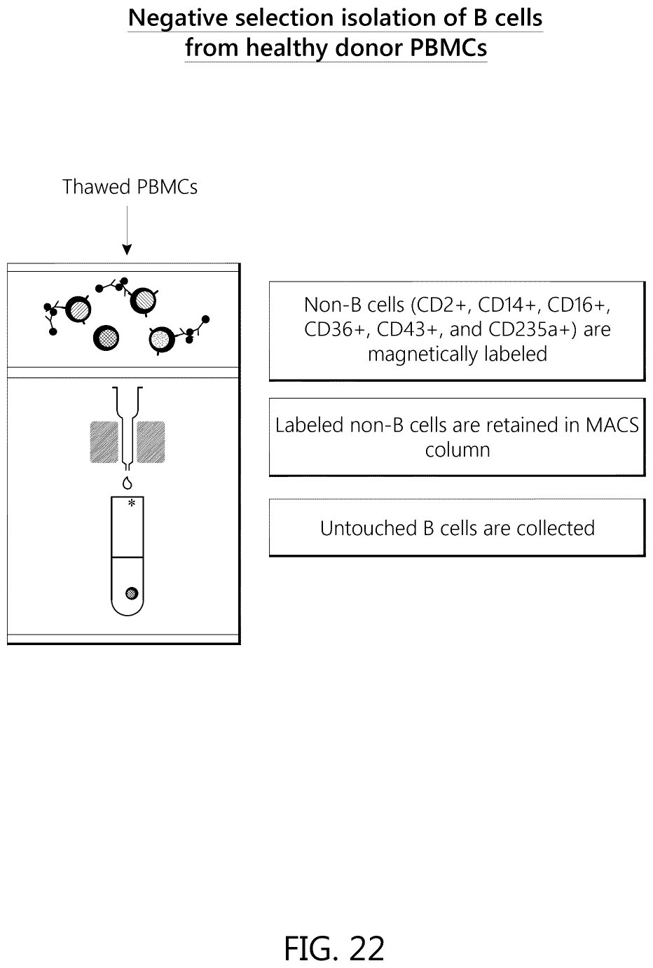

The strategy for homologous directed repair using AAV repair templates are shown in FIGS. 18 and 20. Briefly, peripheral blood mononuclear cells (PBMCs) collected from CD34+ negative selection flow-through from whole blood of healthy male donors post hematopoietic stem cell mobilization were purchased (FIG. 22). CD19+ B cells were isolated from PBMCs by negative selection using a human B cell isolation kit (Miltenyi Biotec®, Auburn, CA) and cultured in Iscove's modified Dulbecco's medium (IMDM, Thermo Fisher Scientific®) supplemented with 10% fetal bovine serum and 55 pM beta-mercaptoethanol at 1-1.5×10⁶ cells/ml. B cells were activated with 100 ng/ml of recombinant human MEGACD40L® (Enzo Life Sciences®), 1 pg/ml of CpG oligodeoxynucleotide 2006 (Invitrogen®), 50 ng/ml of IL-2 (Peprotech®), 50 ng/ml of IL-10 (Peprotech®) and 10 ng/ml of IL-15 (Peprotech®) for 48 hours. Cells were then electroporated with Cas9 RNP complexes using the Neon Transfection System (ThermoFisher Scientific®) as follows. Cells were washed with PBS and resuspended in Neon Buffer T. 30.5pmol Cas9 RNP per 3×10⁵ cells was added to the resuspension so that the final cell density was 3×10⁷ cells/ml. Cells were then electroporated (1700V, 20 ms, 1 pulse) in 10-μl Neon tips, and then transferred into pre-warmed B cell culture medium with MEGACD40L®, CpG, IL-2, IL-10 and IL-15 and cultured at 1.5×10⁶ cells/ml. For samples transfected with an ssODN donor template, ssODN was added concurrently with Cas9 RNP at 30 pmol per 3×10⁵ cells, unless otherwise specified. For samples transduced with AAV, AAV was added to the culture immediately after electroporation. The added AAV volume was 20% of the cell culture volume, unless otherwise specified. Culture volume was doubled 24 hours after electroporation, and medium was replenished every two to three days thereafter to maintain a cell density of 1×10⁶ cells/ml.

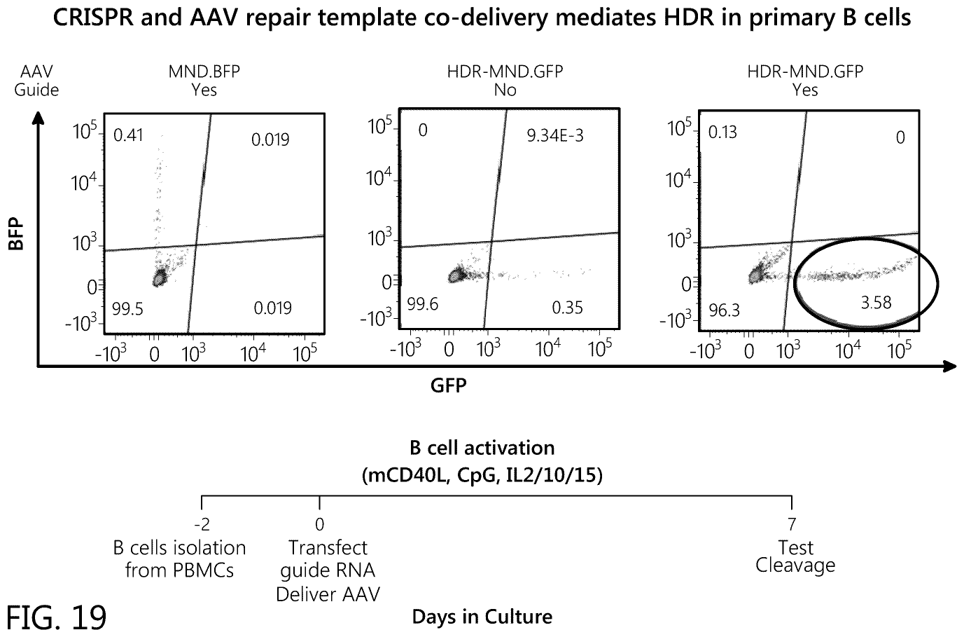

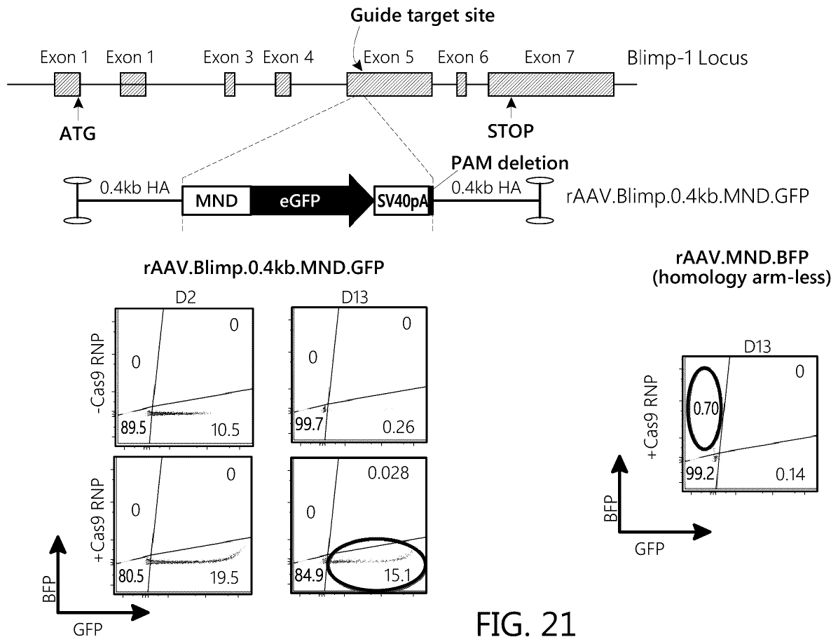

To test HDR using the RNP-AAV approach, B cells were transduced under 3 conditions: (1) PRDM1 RNPs and AAV vector lacking PRDM1 homology arms (MND.BFP), (2) no RNP and AAV vector with PRDM1 homology arms (HDR-MND.GFP) and (3) PRDM1 RNPs and AAV vector with PRDM1 homology arms (HDR-MND.GFP). Shortly after transfection and transduction and following plasmablast differentiation, the degree of BFP and GFP were quantified in B cells using flow cytometry to determine the HDR frequency. As expected, AAV6-driven expression is evident at day 2 in all conditions, but stable GFP expression is only detectable in the condition that had matching RNPs and repair templates. As shown in FIGS. 19 and 21, this initial experiment shows that CRISPR and AAV repair template co-delivery mediates high-efficiency HDR in primary B cells.

### Alternative 5: Optimization of Plasma Cell Differentiation.

Another required step for edited plasma cell generation includes B cell expansion. Experimental set up is shown in FIG. 20. As shown in FIG. 22, B cells are negatively selected from healthy donor PBMCs. B cells were obtained using the B Cell Isolation Kit II from Miltenyi Biotec®. The Non-B cells (CD2⁺, CD14⁺, CD16⁺, CD36⁺, CD43⁺, and CD235a⁺) are magnetically labeled and labeled non-B cells are retained in MACS column. The untouched B cells are then collected.

The culture and editing protocol comprises culturing isolated B cells in Mega-CD40L®⁺ CpG⁺ IL-2⁺ IL-10⁺ IL-15 at 1.5×10⁶ cells/ml for 48h. This is then followed by editing, transfecting and transducing the cells, which are then reseeded at 1.0×10⁶-1.5×10⁶ cells/ml on day 2. The cells are then split on day 3, then every 2-3 days thereafter. The stimulation conditions are optimized for B cell activation, proliferation and RNA/protein delivery by electroporation with a naïve/memory B cell phenotype.

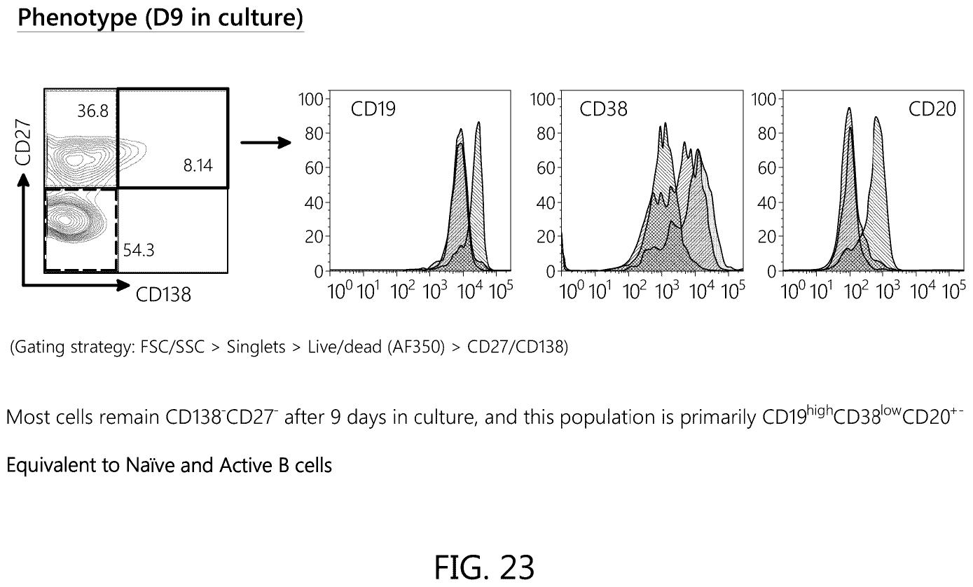

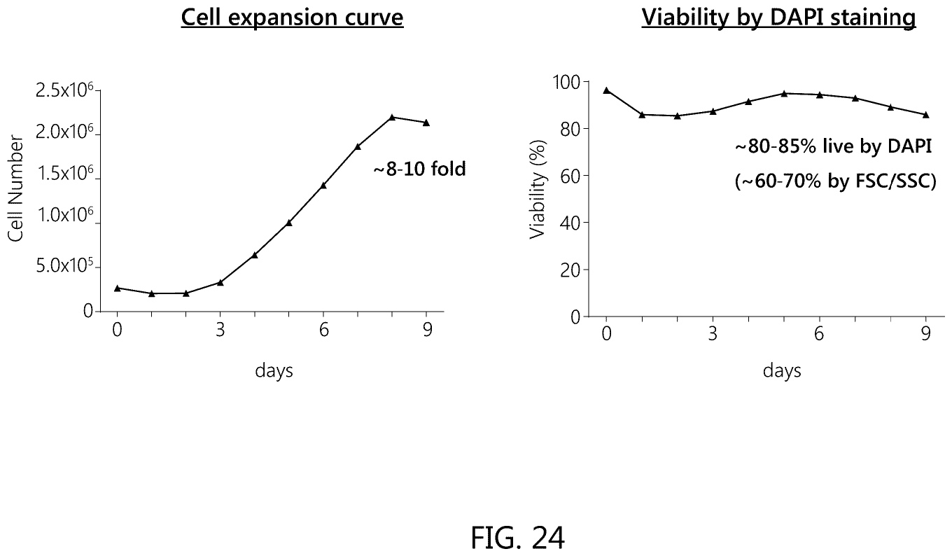

The cells were then phenotyped at the end of the expansion phase at day 9 during the culture. Shown in FIG. 23 is a flow cytometry analysis, which was gated for CD27 and CD138. Most of the cells remained CD138−CD27− after 9 days in culture, and this population was primarily CD19^{high}CD381^{low}CD20⁺, which is equivalent to naïve and activated B cells. The number of B cells were also shown to be at 8-10 fold greater at day 9 and were shown to have a viability of about 80-85% (FIG. 24).

B Cell Differentiation into Long-Lived Plasma Cells

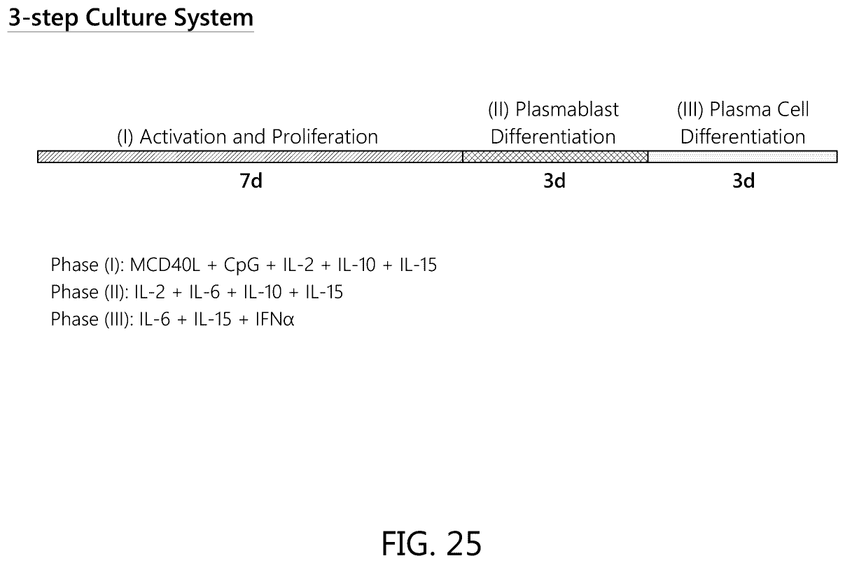

Cells were differentiated using a three step culture system as shown in FIG. 25. Activation and proliferation steps included addition of MCD40L+CpG+IL-2+IL-10+IL-15 to the culture for the first 7 days. Afterwards, at phase II, IL-2+IL-6+IL-10+IL-15 was added to the culture for plasmablast differentiation for the next three days. The final step, phase III, included addition of IL-6+IL-15+IFNα to allow for plasma cell differentiation. The cells were obtained and washed between the phases before addition of the culture additives.

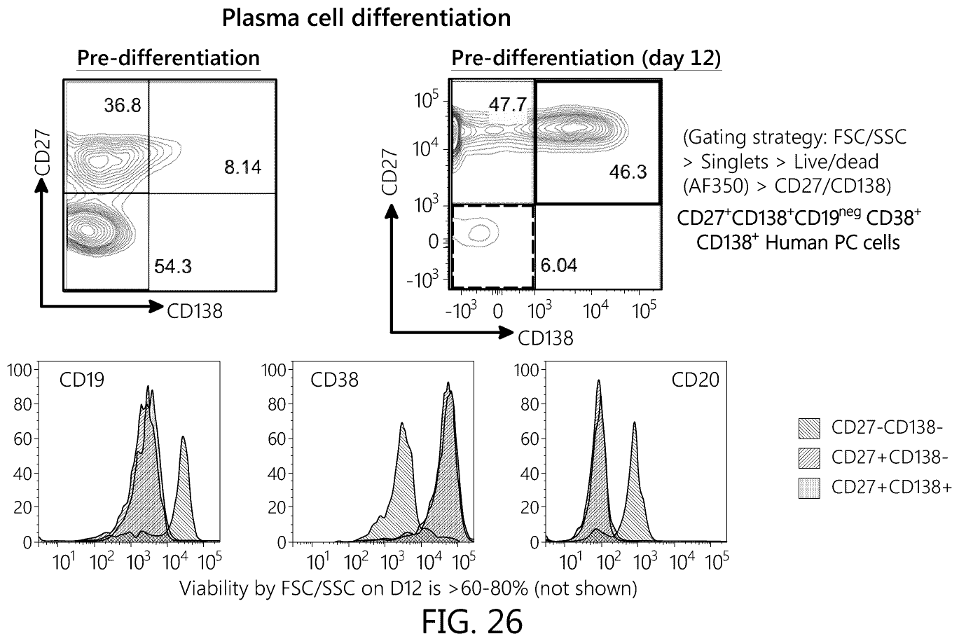

The cells were tested before and after the B cell differentiation steps. Cells were subjected to FACs analysis gated for CD27 and CD138. In comparison of the pre differentiated and the post differentiated cells, at day 12, there is an increase of CD27+CD138+CD19neg CD38+CD138+Human PC cells (FIG. 26). There is also an increase of viability of the cells by FSC/SSC on day 12 which was greater than 60-80%.

In summary, the alternatives provided herein established methods for efficient/non-toxic genome editing in primary human B cells using RNPs, and methods for efficient HDR (>30% using RNP and short ODN). There is also initial demonstration of HDR-mediated introduction of larger 2-4 Kb expression cassettes (using RNP with long ODN or AAV) which has not previously been seen before for B cells. Furthermore, the alternatives have established sequential culture systems to generate and maintain human plasma cells.

### Alternative 6: Demonstration of Quantifiable Secretion of an Exogenous Protein in an Animal Model

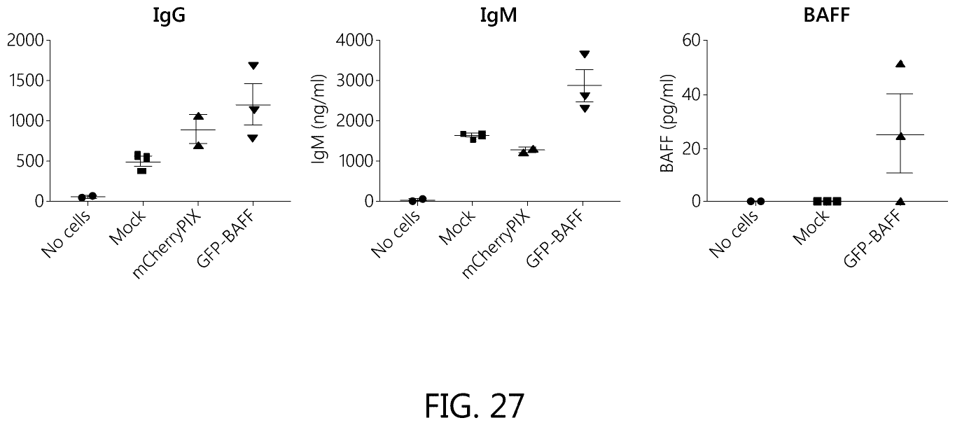

Naïve human B cells were isolated from PBMCs, and expanded in B cell activation cocktail (mCD40, CpG, IL2/10/15) for 2 days. At this point, 1 million cells were mock transfected or transfected with CCR5-targeting RNPs and transduced with homology-directed repair templates sufficient to deliver mCherry-Factor IX or eGFP-BAFF to the B cells. Following 5 additional days of activation, the cells were differentiated into plasmablasts for 3 days via incubation with IL2/6/15 and subsequently into plasma cells by incubation with IL6/15 and IFNalpha. 3 million of this population of plasma cells from each condition was implanted into NSG mice (NOD, Cg-Prkcd^{SCID}, Il2rg^{tm1Wjl}/SzJ) using retroorbital injection. Following 7 days, a blood draw was taken from each mouse and the quantity of human IgG, human IgM and human BAFF was determined by ELISA. As observed in the left two panels, plasma cells expressing BAFF produce more IgM and IgG than unedited cells. Furthermore, human BAFF is quantifiable in these animals implanted with BAFF-edited plasma cells. (FIG. 27)

Data Showing Sustained In Vivo Engraftment of Gene-Edited Human Plasma B Cells and Increased Engraftment of Edited B Cells that Express BAFF (Introduced by HDR Based Gene Editing).

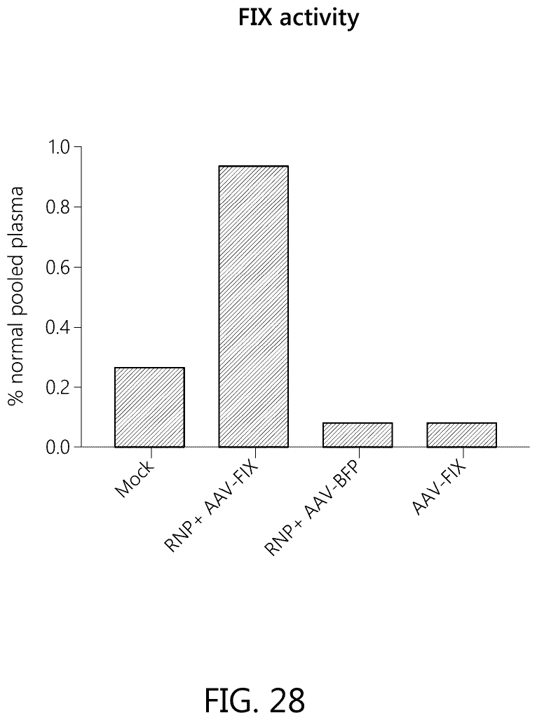

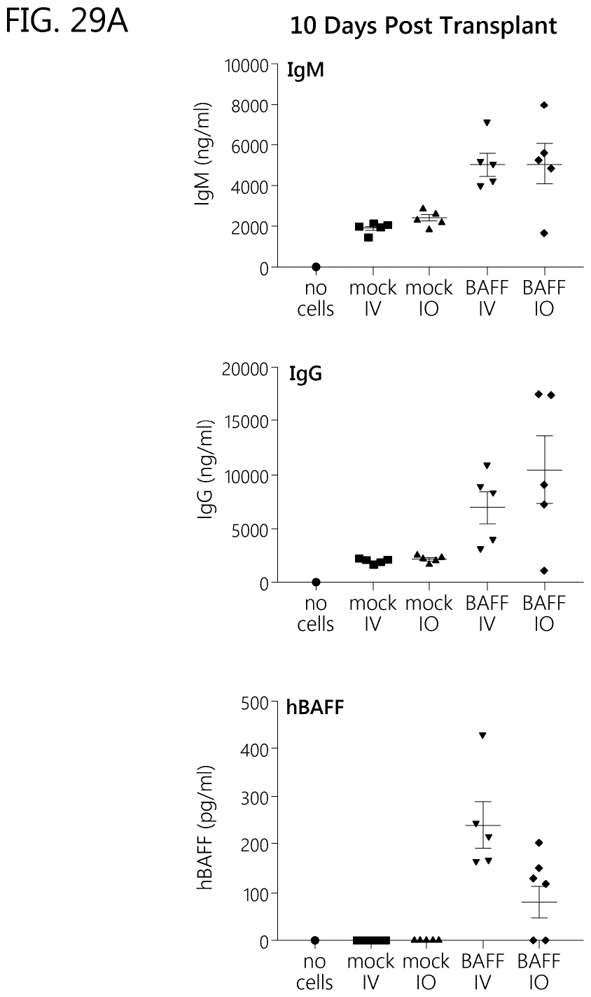

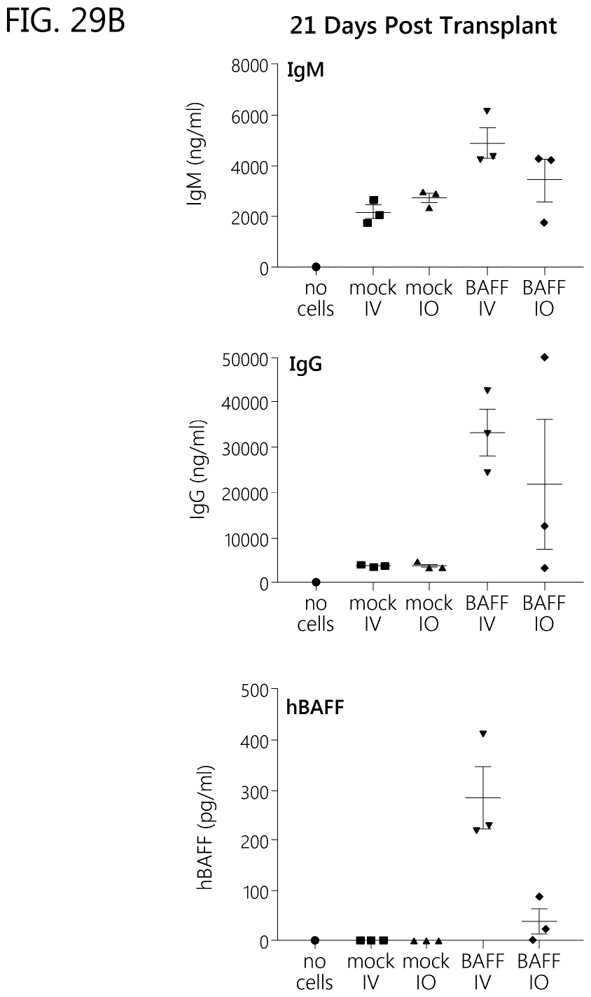

Naïve human B cells from peripheral blood mononuclear cells were expanded in B cell activation cocktail (mCD40, CpG, IL2/10/15) for 2 days. At this point, 1 million cells were mock transfected or transfected with CCR5-targeting RNPs and transduced with homology-directed repair templates sufficient to deliver eGFP-BAFF to the B cells. Following 5 additional days of activation, the cells were differentiated into plasmablasts for 3 days via incubation with IL2/6/15 and subsequently into plasma cells by incubation with IL6/15 and IFNalpha. 10 million cells from this population from each condition was adoptively transferred into NSG mice (NOD, Cg-Prkcd^{SCID}, Il2rg^{tm1Wjl}/SzJ) via either retroorbital or intraosseous injection. At Day 10 (Figure. 29A) and 21 (FIG. 29B) following transplantation, serum abundance of human IgM, human IgG and hBAFF was measured by ELISA. Comparison of FIG. 29A and FIG. 29B show sustained expression of human proteins in murine serum between 10 and 21 days, indicating that edited human plasma cells are stably engraftable in mice. Further, these data show that secretion of hBAFF by gene-edited plasma cells enhances PC engraftment. (FIG. 28).

### Alternative 7: Demonstration of the Ability of B Cells to Produce Functionally Active Factor IX

Naïve human B cells were isolated from PBMCs, and expanded in B cell activation cocktail (mCD40, CpG, IL2/10/15) for 2 days. At this point, 1 million cells were mock transfected, RNP transfected, AAV transduced or transfected with CCR5-targeting RNPs and transduced with homology-directed repair templates sufficient to deliver Factor IX to the B cells. Following 5 additional days of activation, the cells were differentiated into plasmablasts for 3 days via incubation with IL2/6/15 and subsequently into plasma cells by incubation with IL6/15 and IFNalpha. 6 days prior to the termination of the study, 1 million cells per mL from each condition were incubated with Vitamin K1 (5 ug/mL). The supernatants from all conditions were subjected to a chromogenic assay of Factor IX activity. Using this assay, it was found that plasma cells edited to integrate exogenous Factor IX produce enzymatically active protein.

### Alternative 8: Assessing the Impact of Disruption and Survival

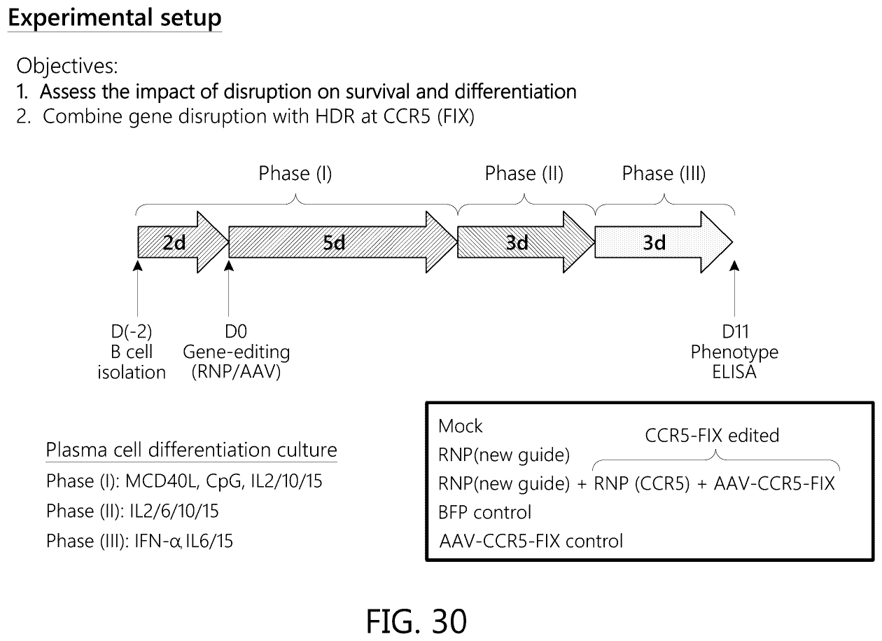

The objective of the further experimentation was to determine the impart of gene disruption on the survival and differentiation of the cells. Also performed, was a combinatorial gene disruption with HDR at CCR5 (FIX) (FIG. 30).

For the first phase at −2 days, B cells were isolated. At day 0, the cells were edited with a RNP/AAV vector (phase 1). In the first phase, the plasma cell differentiation culture comprised MCD40L, CpG, and a mixture comprising IL2/IL10/IL15. The first phase of plasma cell differentiation was carried out for 5 days. At phase 2 (3 days), the plasma cell differentiation culture comprised IL2/IL6/1115. At phase 3 (3 days), the plasma cell differentiation culture comprised IFN-α, IL6 and IL15.

### Alternative 9: Long Lived Human Antibody Production in Mice

Primary B cells were expanded in culture and edited as described. Following differentiation into plasma cells, 10 million were injected into NSG mice using either retro-orbital (RO) or intra-osseous (IO) injection. The mice were bled at the indicated time points. Human IgG (IgM not shown) was quantified using ELISA. Each dot represents data from an individual recipient mouse at the indicated time point. These data show that gene edited human plasma cells can live for long periods in this murine model despite the absence of human cytokines. These data also show that autocrine production of human BAFF can further promote sustained survival of gene edited PCs.

Several groups of mice were under four editing conditions: group 1) mock unedited cells; group 2) cells edited with the CCR5-RPN+AAV-GFP-BAFF; group 3) Mock unedited cells and group 4) CCR5-RNP+AAV-GFP-BAFF. Groups 1 and 2 were administered the cells by retro-orbital injection (10E+06 cells/animal) and groups 3 and 4 were administered the cells by intra-osseous injection (10E+06 cells/animal). Each group had 5 mice. As shown, the sets of cells from left to right under the “no cells” area are the week one, week 3, week 5, week 5, week 7, week 9, week 11, week 13, week 15, week 17 and week 19 cells cells, this order is kept throughout the x-axis for mock IO, BAFF IO, Mock RO and BAFF RO. The mock unedited cells (Group 1) have minimal expression of the IgG. However, cells of group 2 have an increase of IgG expression that peaks at week 3 and 5. The cells of Group 3 (mock unedited) also show minimal expression of IgG, whereas the cells of Group 4 have an increase of IgG that peaks at week 3 and 5. As shown, cells administered with the CCR5-RNP with the AAV-GFP-BAFF with either tero-orbital injection or intra-osseous injection show long-lived human antibody production in mice.

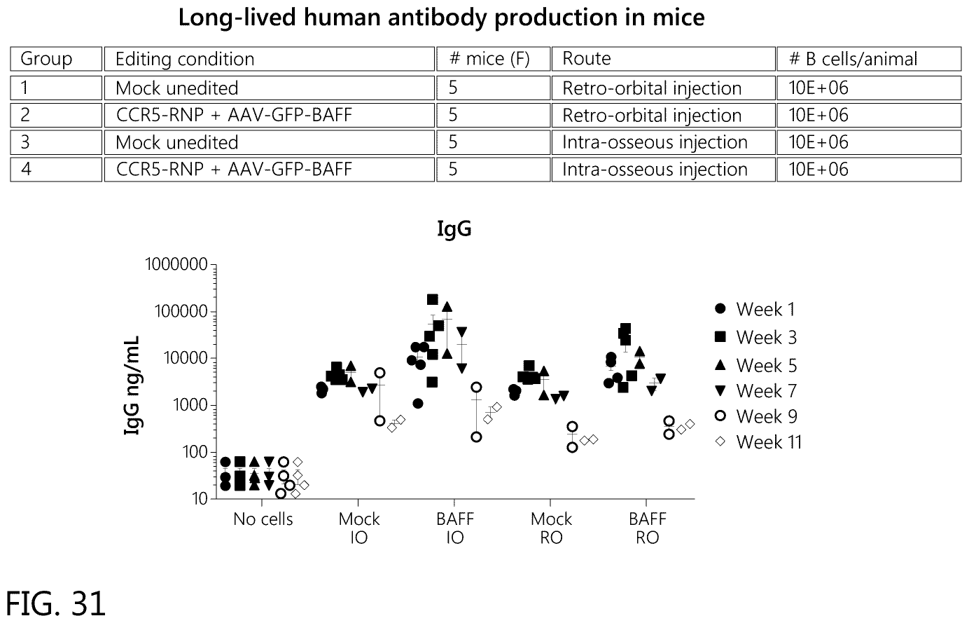

Primary B cells were expanded in culture and edited as described. Following differentiation into plasma cells, 10 million were injected into NSG mice using either retro-orbital (RO) or intra-osseous (IO) injection. The mice were bled at the indicated time points. Human IgG (IgM not shown) was quantified using ELISA. Each dot represents data from an individual recipient mouse at the indicated time point. These data show that gene edited human plasma cells can live for long periods in this murine model despite the absence of human cytokines. These data also show that autocrine production of human BAFF can further promote sustained survival of gene edited PCs. (FIG. 31).

### Alternative 10: Expression of Human IL6 in Mice Confers a Growth Advantage for Transplanted Ex Vivo Differentiated Human Plasma Cells.

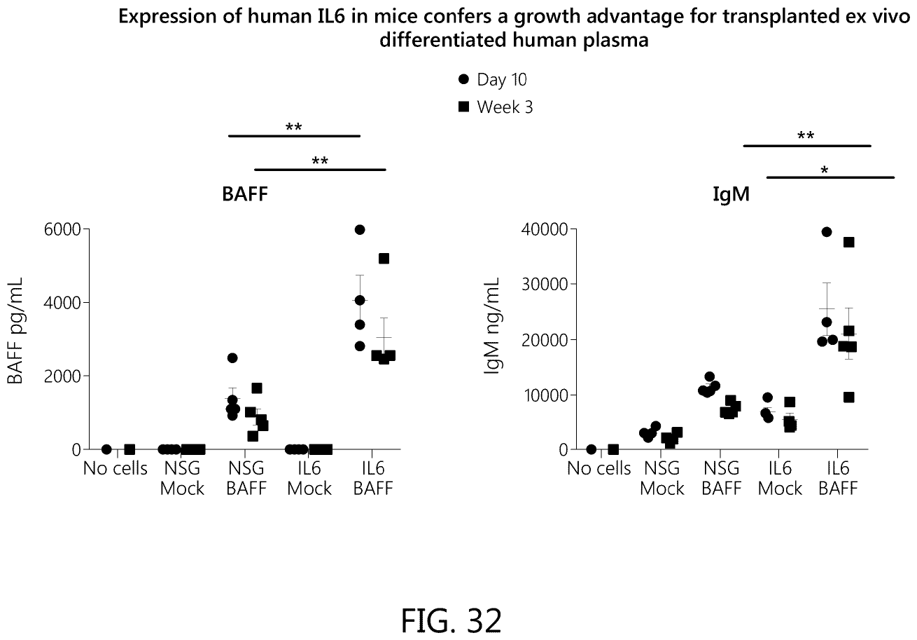

Neonatal NSG mice were injected with lentiviral vectors expressing human IL6 and used for adoptive cell transfer at approximately 8-12 wk of age. Primary human B cells were expanded in culture and edited as described (mock or BAFF delivered to the CCR5 locus). Following differentiation into plasma cells, 10 million were injected into NSG mice using retro-orbital (RO) injection. The mice were bled at the indicated time points. Human BAFF or IgM was quantified using ELISA. Each dot represents data from an individual recipient mouse at the indicated time point. These data show that human IL6 secretion can confer an in vivo survival advantage to transplanted human plasma cells and that this can synergize with autocrine expression of human BAFF in gene edited cells (FIG. 32).

Alternative 11: High IL6 Expression Correlates with High Plasma Cell Secretion.

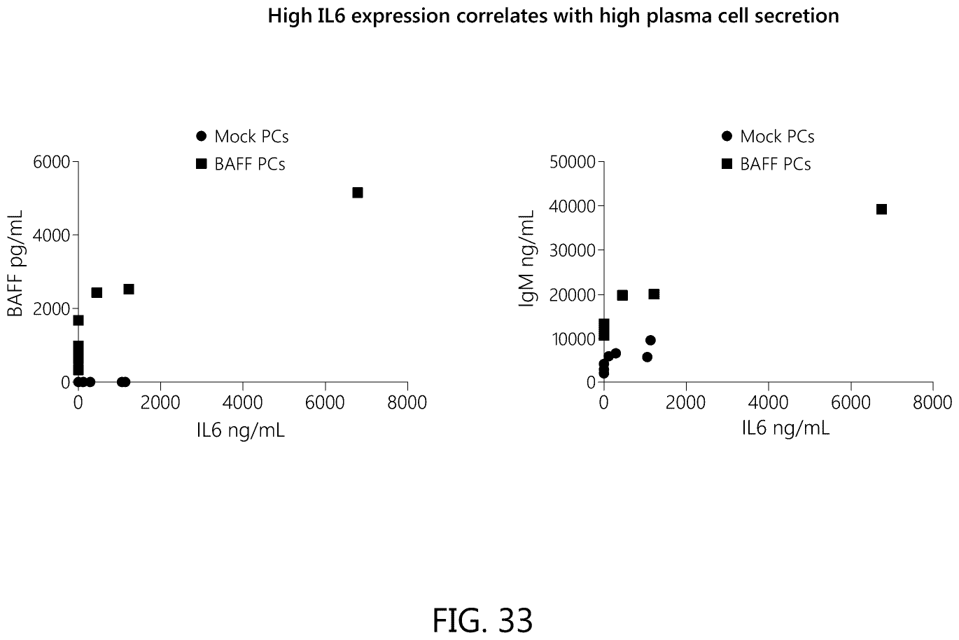

Neonatal NSG mice were injected with lentiviral vectors expressing human IL6 and used for adoptive transfer of human cells at 8-12 wks of age. Primary human B cells were expanded in culture and edited as described (mock or BAFF delivered to the CCR5 locus). Following differentiation into plasma cells, 10 million were injected into NSG mice using either retroorbital (RO) injection. The mice were bled at the indicated time points. Human IL6, BAFF or IgM was quantified using ELISA. Each dot represents data from an individual recipient mouse at the indicated time point. These data demonstrate that the levels of human IL6 directly correlated with the levels of human protein production by the gene-edited transplanted cells, and likely the number of surviving long-lived plasma cells (FIG. 33).

Alternative 12: In Vitro Human APRIL Treatment Correlates with Increased Plasma Cell Antibody Secretion In Vivo.

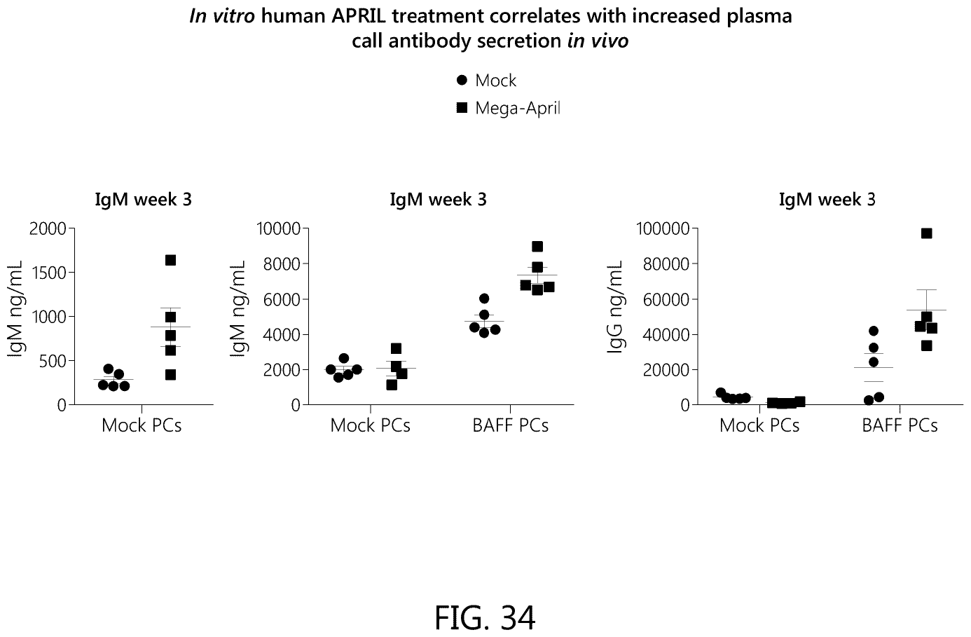

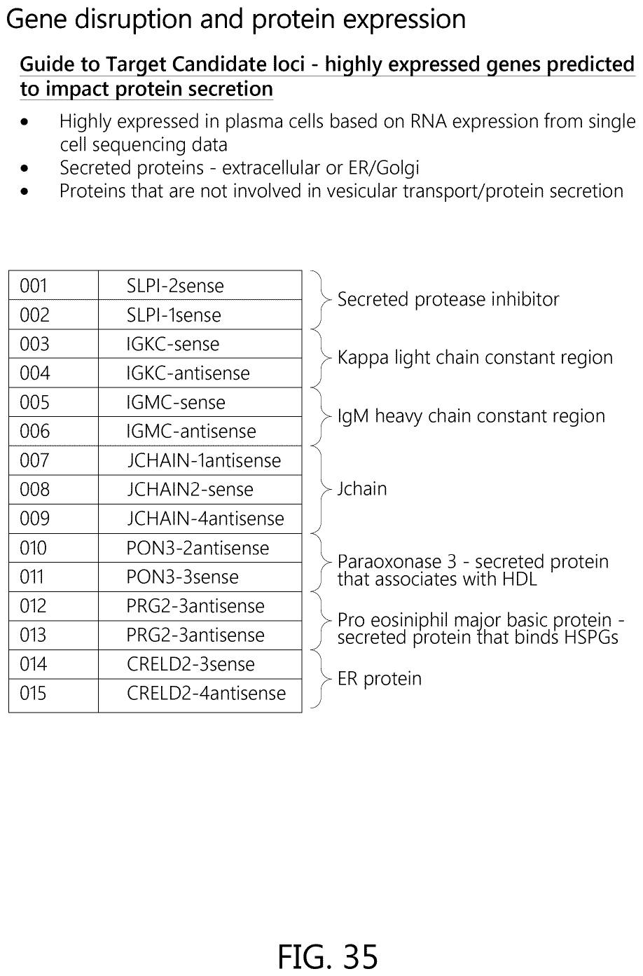

Primary B cells were expanded in culture and edited so that BAFF or mock is delivered to the CCR5 locus. During differentiation into plasma cells, the cells were cultured in the presence of trimeric APRIL (Mega-April™) or mock. After 12 days in culture, IgM and IgG secretion was quantified by ELISA. Each dot is from an experiment from an individual donor. These data show that April promotes antibody production in in vitro derived gene-edited human plasma cells. (FIG. 34). Shown in FIG. 35, are the results of the gene disruption and protein expression.

### Alternative 13: Targetin2 of Abundant Plasma Cell Genes Using CRISPR.

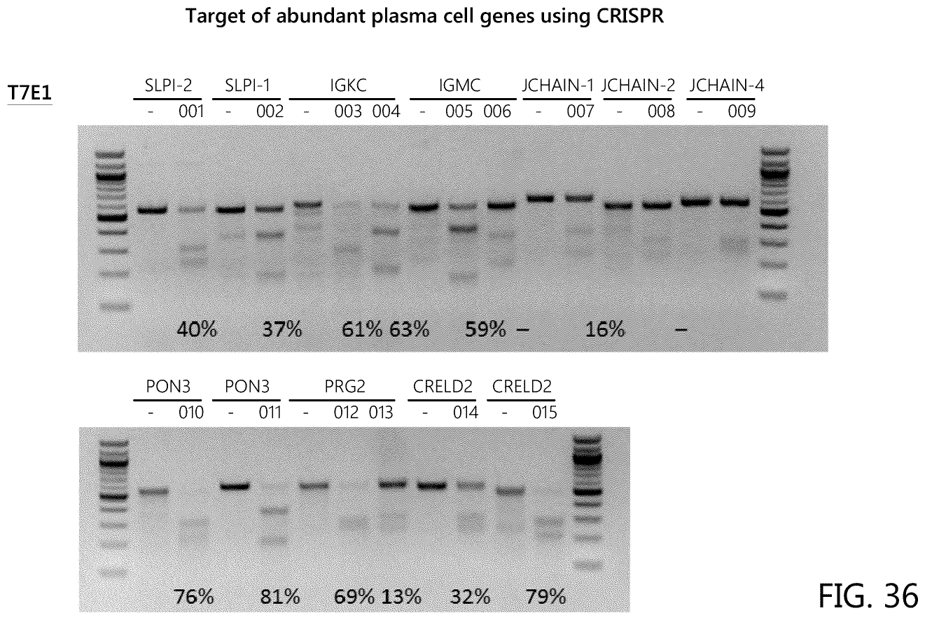

Primary human B cells were expanded in culture and edited to disrupt the indicated loci. 5 days following transfection, genomic DNA was harvested for amplification and quantification of insertions and deletions using a T7 endonuclease I cleavage assay. As demonstrated and quantified, multiple guides successfully disrupt the target loci. (FIG. 36).

### Alternative 14: Multiplexed 2Enome Editing to Introduce FIX and Increase Protein Secretion in Human Plasma Cells.

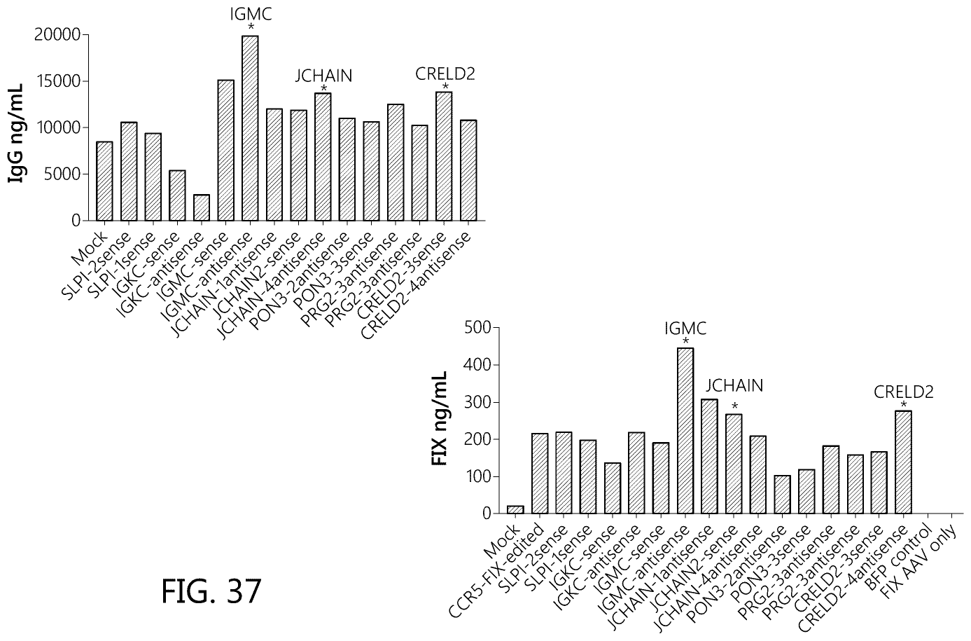

Primary human B cells were expanded in culture and edited to disrupt the indicated loci. Using multiplexed editing, FIX was also introduced into the CCR5 locus by homology-directed repair. After three-step differentiation into plasma cells, IgG and FIX were quantified by ELISA. These data show that knocking out a subset of highly expressed genes, including the heavy chain (IGMC), JCHAIN and CRELD2, production of IgG and FIX in gene edited plasma cells can be increased. (FIG. 37).

### Alternative 15: Knock-Out of BANK1 Promotes Plasma Cell Differentiation and Antibody Production.

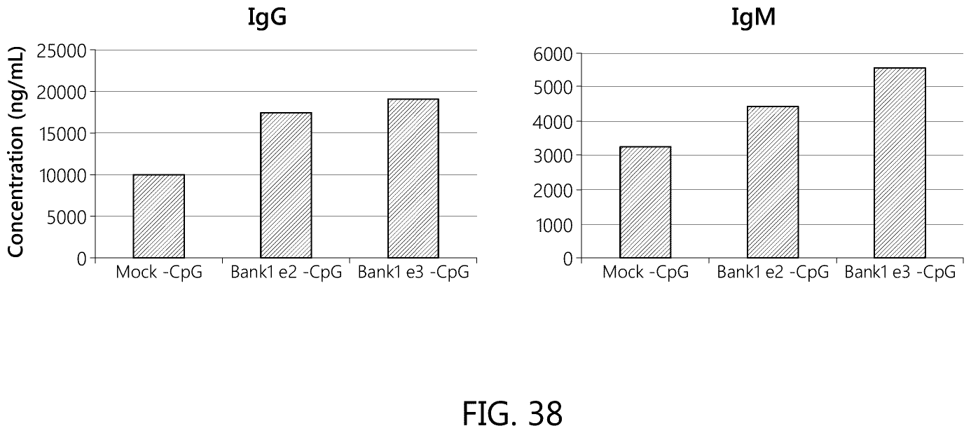

Primary human B cells were expanded in culture and edited to disrupt BANK1. After three-step differentiation into plasma cells, IgG and IgM were quantified by ELISA. These data show that knocking out the B cell developmental regulator BANK1, IgG and IgM can lead to increased production of in gene edited plasma cells. (FIG. 38). Consistent with these data disruption of BANK1 also promotes PC differentiation.

Alternative 16: Long-Lived Plasma Cells Secrete More immuno2lobulin on a Per Cell Basis.

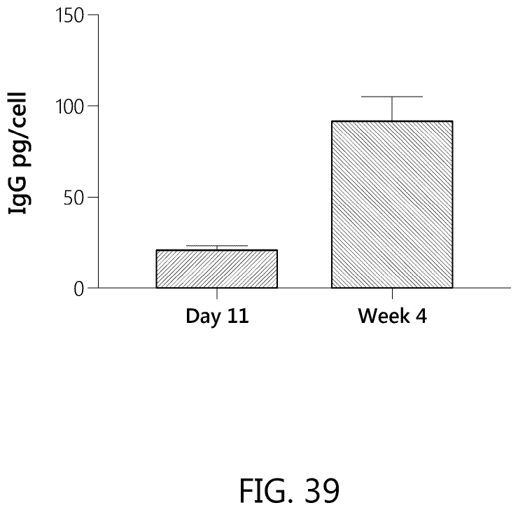

Primary human B cells were expanded in culture. After three-step differentiation into plasma cells, plasma cells (CD138 enriched cells) were cultured for an additional 3 weeks in the presence of IFN-beta, IL6, IL15 and trimeric APRIL. Three days following a media change, IgG was quantified by ELISA at day 11 and at 4 weeks. These data show that long-lived plasma cells produce substantially more IgG on a per cell basis (n=2 donors). (FIG. 39).

### Alternative 17. In Vivo Delivery of Human APRIL or IL6 Enables Increased Lon2-Term Secretion by Edited Long-Lived Plasma Cells

Neonatal NSG mice were injected with lentiviral vectors expressing human IL6 and/or APRIL and used for adoptive cell transfer at approximately 8-12 wk of age. Primary human B cells were expanded in culture and edited as described (mock or BAFF delivered to the CCR5 locus). Following differentiation into plasma cells, 10 million were injected into NSG mice using retro-orbital (RO) injection. The mice were bled at the indicated time points. Human BAFF was quantified using ELISA. Each dot represents data from an individual recipient mouse at the indicated time point. These data show that human IL6 and APRIL secretion can confer increase the long-term production of an exogenously delivered protein (BAFF) by edited human plasma cells.(Figure

### Alternative 18: Engineering Protein-Secreting Plasma Cells by Homology-Directed Repair in Primary Human B Cells

A method is presented for high-efficiency, homology-directed genome editing in primary human B cells for the purposes of producing therapeutic proteins.

B cells differentiate into long-lived plasma cells that provide humoral immunity by secreting large quantities of antibodies. The ability to engineer primary human B cells to secrete a de novo protein may allow the creation of novel plasma cell therapies for protein deficiency diseases and other clinical applications. To achieve this goal, methods for efficient genome editing of primary B cells isolated from peripheral blood were developed, followed by ex vivo differentiation into plasma cells. By delivering CRISPR/Cas9 ribonucleoprotein (RNP) complexes under conditions of rapid B cell expansion, site-specific gene disruption at multiple loci in primary human B cells was achieved (with editing rates up to 94%). This method was first used to modulate plasma cell differentiation by disrupting key developmental regulatory genes. Next, RNPs were co-delivered with either single-stranded DNA oligonucleotide or adeno-associated viruses containing a homologous repair template. Using either delivery method, targeted sequence integration at high efficiency (up to 40%) via homology-directed repair was achieved. This method enabled engineering of plasma cells to secrete B-cell activating factor (BAFF) or factor IX (FIX) at high levels. These results highlight the utility of genome editing in studying human B cell biology and demonstrate a novel strategy for modifying human plasma cells to secrete therapeutic proteins.

Long-lived plasma cells stably reside in the bone marrow for decades and secrete large quantities of antibodies. Consequently, plasma cells engineered to produce de novo proteins have the potential to be curative therapies for protein deficiency diseases, prophylaxis for infectious diseases and many other applications. However, the development of plasma cell therapeutics has been limited by technical challenges in the in vitro modification, culture, expansion and differentiation of primary human B cells. B cells can be transduced at high rates by recombinant adenovirus or Epstein-Barr virus (EBV) vectors, which deliver transgenes as episomes. However, episomal DNA expression is lost over time, limiting use of these vectors in applications that require long-term transgene expression. Unlike non-integrating vectors, gamma retrovirus (yRV) and lentivirus (LV) randomly integrate into the host genome and can be used to introduce stably expressing transgenes. However, these vectors are inefficient at transducing primary human B cells. LV that employ alternative envelopes, including that of baboon retrovirus, measles virus, or gibbon-ape leukemia virus exhibit higher B cell transduction rates (up to ˜50%), but have low viral titers that make large-scale production challenging. Because yRV and LV vectors do not efficiently transduce B cells while transduction by non-integrating vectors results in only transient transgene expression, neither platform is currently effective for delivering long-term expression of exogenous genes to B cells on a therapeutic scale.

An alternative method for introducing stable protein expression is genome editing via homology-directed repair (HDR). Following cleavage by an engineered site-specific nuclease, DNA double-strand breaks were resolved through non-homologous end joining (NHEJ), an error-prone DNA repair pathway that typically leads to variable insertions or deletions (indels), or HDR, which repairs DNA by copying a homologous donor template. Delivery of exogenous DNA flanked by DNA homologous to the genomic sequence around the break site can lead to incorporation of the exogenous sequence in a site-specific manner. HDR-mediated genome editing in B cells may have several advantages over viral vector transduction for therapeutic applications, including decreased risk of insertional mutagenesis and sustained transgene expression. Many have recently achieved high-efficiency HDR delivery of therapeutic transgenes to hematopoietic cells including primary human T cells and hematopoietic stem cells, but similar approaches are yet to be applied in modification of primary human B cells.

The clustered regularly interspaced short palindromic repeats (CRISPR)/CRISPR-associated protein 9 (Cas9) system is an RNA-guided nuclease platform that is easily engineered to efficiently target specific sites in the genome for cleavage, generating double-strand DNA breaks. The use of site-specific nucleases for gene disruption or HDR in B cells is currently limited to transformed or lymphoma-derived cell lines and murine models and has required plasmid- or LV-based CRISPR/Cas9 delivery. Described herein is a method of high-efficiency genome editing in human peripheral blood B cells (75-90% gene disruption or 10-40% HDR) by delivering CRISPR/Cas9 ribonucleoprotein (RNP) complexes alone or in combination with single-stranded DNA oligonucleotide (ssODN) or adeno-associated virus (AAV) repair templates, respectively. In the alternatives herein it is shown that edited primary B cells can subsequently be differentiated in culture into plasma cells that produce physiological doses of therapeutic proteins including human Factor IX (FIX).

### Conditions for Expansion of Primary Naïve Human B Cells

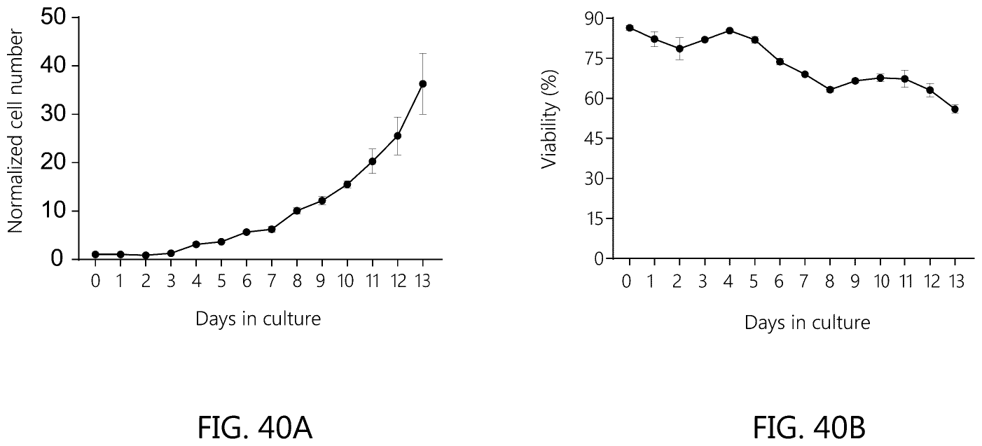

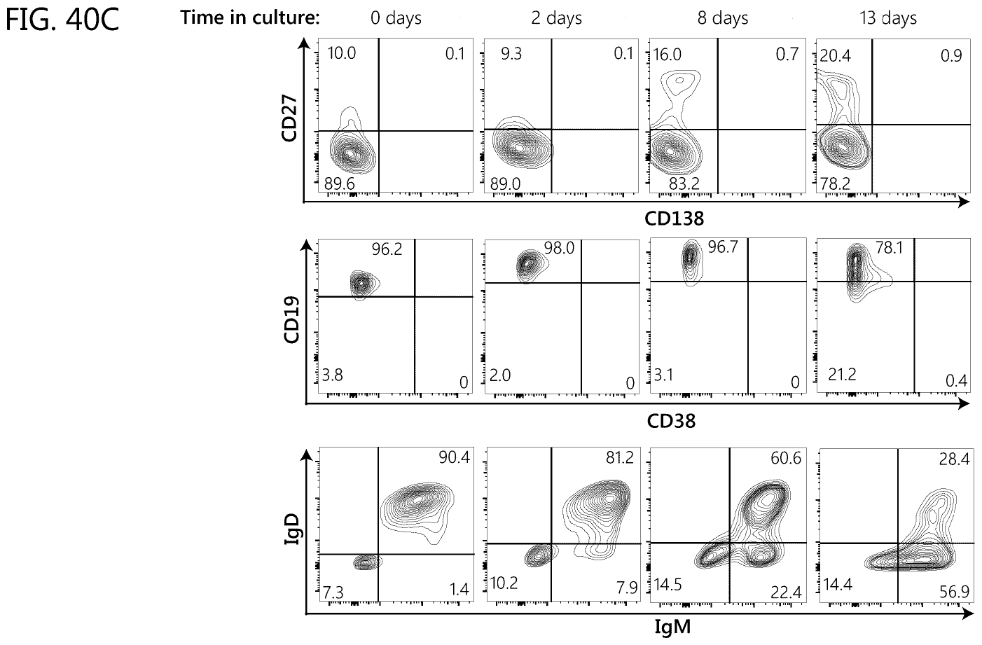

Rapid cell cycling and/or persistence in the S/G2 phases of the cell cycle promote HDR in both cell lines and primary hematopoietic cells. Based on previous reports demonstrating rapid expansion of primary human B cells ex vivo, a combination of stimulants (hereafter called “B cell activation cocktail”) was initially used that included artificially oligomerized CD40 ligand (MEGACD40L®; two linked CD40L trimers) in association with CpG, IL2, IL10, and IL15. Primary human CD19+ B cells from peripheral blood mononuclear cells (PBMCs) were isolated and cultured them for 13 days with this B cell activation cocktail. This approach resulted in a ˜36-fold expansion of B cells (FIG. 40A), while preserving viability at >60% (FIG. 40B). Despite having proliferated over 13 days, most cells maintained a naïve B cell phenotype (CD27⁻CD138⁻CD38^{low/-}CD19^{high}IgM⁺ IgD⁺; FIG. 40C), demonstrating that the B cell activation cocktail facilitates rapid cycling and ex vivo maintenance of naïve primary human B cells.

### Cas9-Mediated Disruption of CCR5 and PRDM1 in Primary Human B Cells

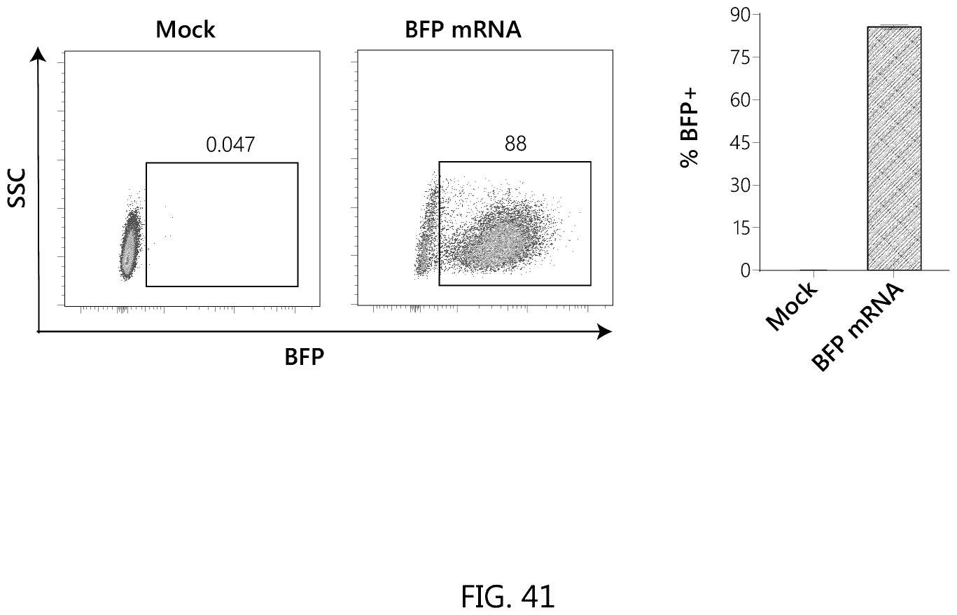

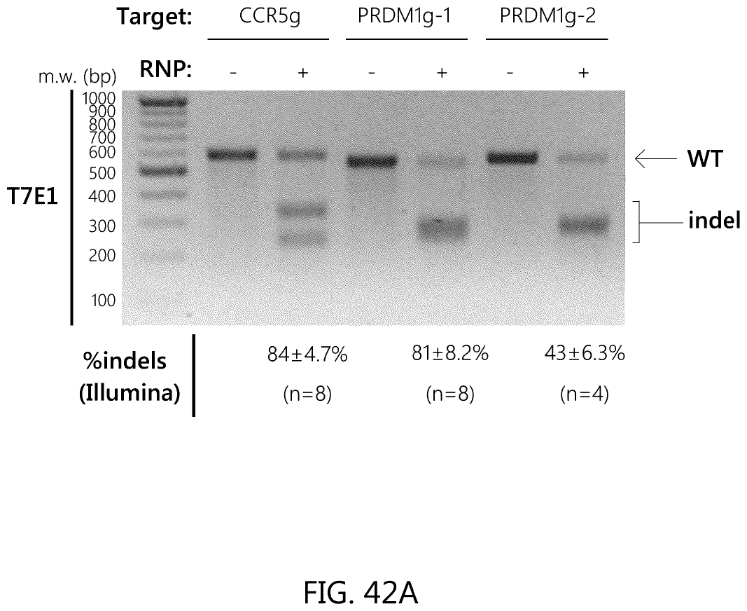

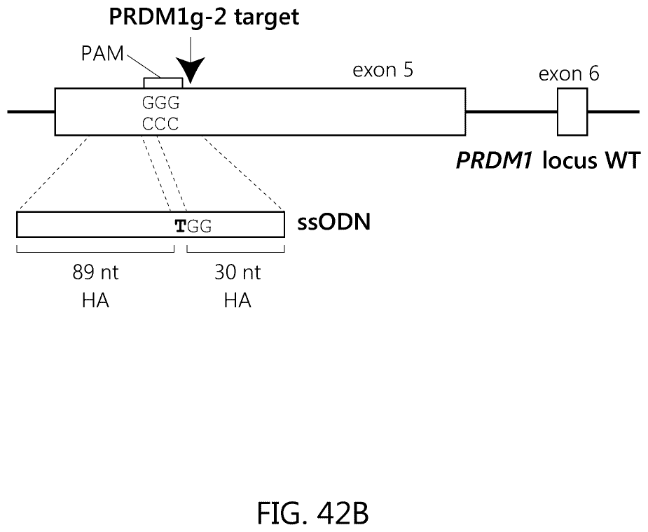

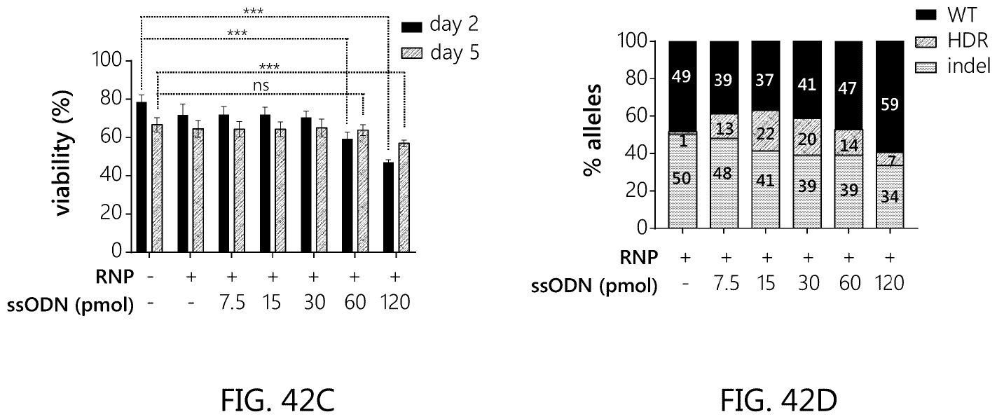

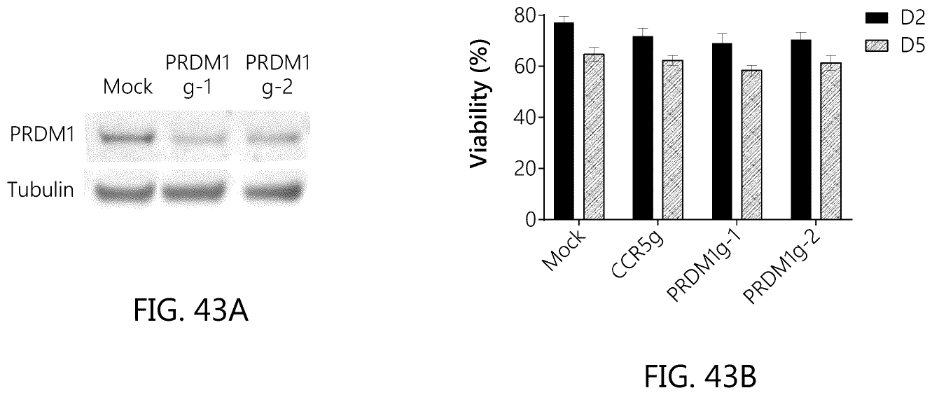

To assess the efficiency of Cas9-induced indels in B cells, CRISPR guide RNAs (gRNAs) were designed that target CCR5 (which is not expressed in human B cells and has no known significance in plasma cell development) or PRDM1 (encoding BLIMP1, a protein required for B cell differentiation into plasma cells). After optimizing electroporation based upon mRNA (FIG. 41), 30 pmol Cas9-guide ribonucleoprotein (RNP) complexes were transfected into B cells, cultured for five additional days under activating conditions and extracted total genomic DNA to assess nuclease-induced indels using the T7 endonuclease 1 assay (FIG. 42). Sequencing confirmed on-target indels and revealed that the RNPs induced high indel frequencies at each guide target site (43-84%; FIG. 42A). Western blot also verified concomitant PRDM1 protein reduction in the PRDM1-expressing TMD8 lymphoma cell line (FIG. 43A). Finally, it was found that B cells remained viable despite these genome modifications (FIG. 43B). These data show that high-efficiency Cas9-mediated gene disruption is achievable in primary human B cells.

HDR-Mediated Single-Nucleotide Substitution at the PRDM1 Locus Using an ssODN Donor Template

Cas9-induced DNA lesions can be seamlessly repaired via the HDR pathway in the presence of a donor template with homology sequences flanking the lesion. An ssODN was initially tested an as a donor template based on promising results in other cell types. A 120-base ssODN was designed containing asymmetric homology arms (89 bases 5′ and 30 bases 3′) that flank the PRDM1g-2 target site, with a single-nucleotide change at the 90^{th} position that mutates the last nucleotide of the protospacer adjacent motif (PAM; GGG to GGT; FIG. 42B). This single nucleotide change was designed to both prevent Cas9-mediated cleavage of the repaired sequence and to serve as a molecular marker for HDR. In this experiment, primary human B cells were activated for two days and then transfected with Cas9 RNP in conjunction with various doses of the ssODN. Two and five days following transfection, cells transfected with <30 pmol ssODN had comparable viabilities to Cas9 RNP transfected control cells (FIG. 42C). In cells receiving 15 or 30 pmol ssODN, sequencing of the PRDM1 target region on day 5 post-transfection revealed 20-22% of alleles had undergone HDR while another 37-41% had indels (FIG. 42D), an overall editing rate marginally higher than that observed in the Cas9 RNP control (FIG. 42D). Thus, ssODN donor templates can be used to achieve high rates of HDR in primary human B cells with low cytotoxicity.

Cas9-Mediated Disruption of Genes that Regulate Plasma Cell Development

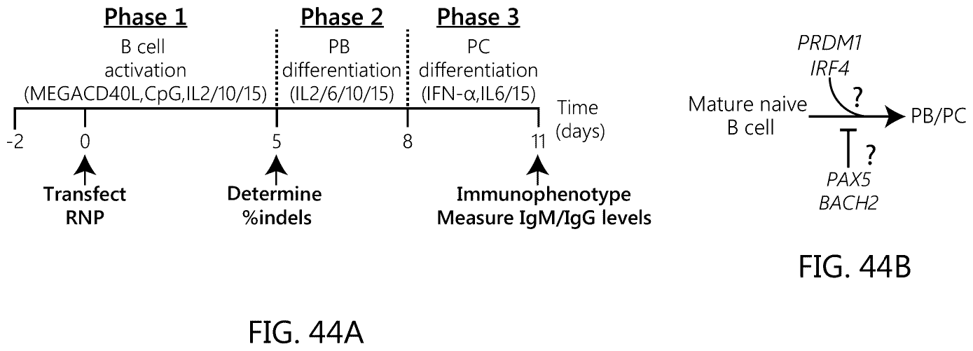

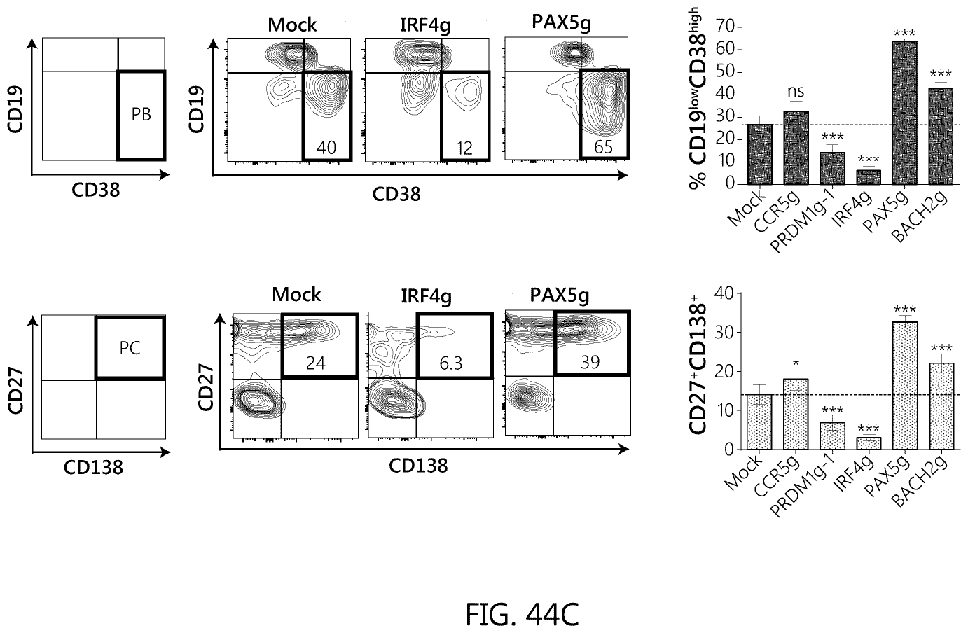

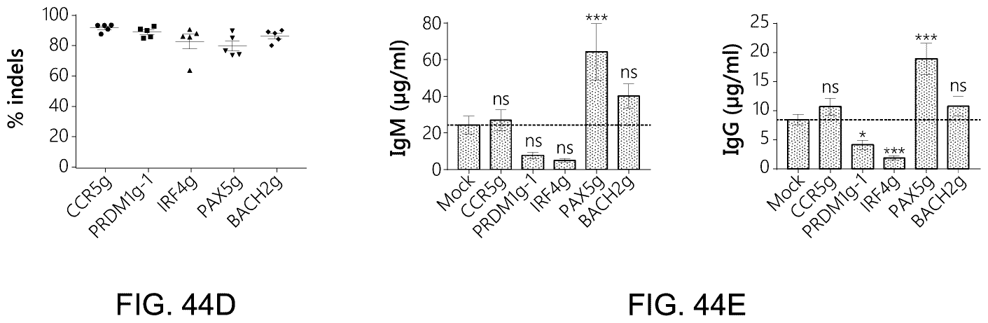

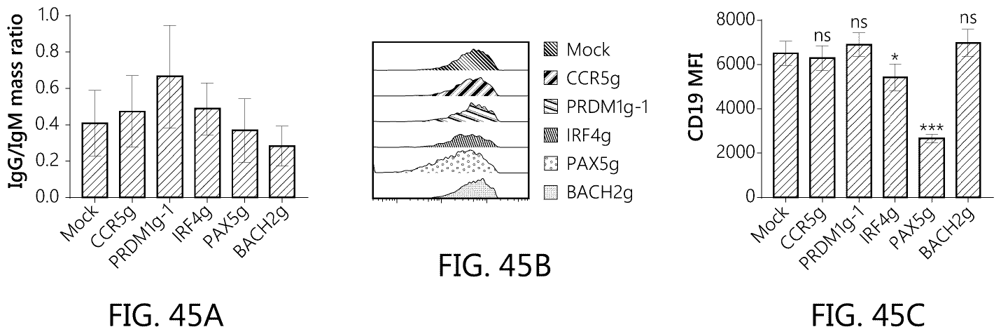

It was next investigated whether Cas9-induced gene disruption can be used to study gene roles in human plasma cell development and antibody production. To do so, an experimental workflow was developed that includes inducing NHEJ-mediated gene disruption in activated naïve human B cells using Cas9 RNPs and subsequently differentiating naïve B cells into plasma cells (CD19^{low} CD38^{high}CD27⁺CD138⁺) using a three-step plasma cell culture system (see FIG. 44A). As a proof of concept, four genes were studied encoding transcription factors previously reported to regulate plasma cell development in murine studies: IRF4 and PRDM1/BLIMP1 are required for plasma cell differentiation (FIG. 44B), while PAX5 and BACH2 antagonize plasma cell differentiation in mice (FIG. 44B). CRISPR guides were designed targeting each of these four genes, and were transfected into primary B cells with Cas9 RNP targeting each gene independently and subsequently induced in vitro plasma cell differentiation (FIG. 44A). As a control, cells were also transfected with Cas9 RNPs targeting CCR5. Following differentiation, significantly lower percentages of CD191^{low}CD38^{high} and CD27⁺CD138⁺ plasma cells were detected in cultures transfected with PRDM1- or IRF4-targeting RNPs compared to cells transfected with CCR5-targeting RNP or mock transfected cells (mean indel percentages=89%, 83% and 92% at the PRDM1, IRF4 and CCR5 target sites, respectively; FIG. 44C, 44D). As antibody secretion is a major function of plasma cells, IgM and IgG levels were measured in cell culture supernatants by enzyme-linked immunosorbent assay (ELISA) and found IgG to be significantly decreased in the PRDM1- and IRF4-targeted B cell cultures (FIG. 44E). Together, as predicted, these data imply that both PRDM1 and IRF4 are required for human plasma cell differentiation and antibody production. Conversely, significant increases in the percentages of plasma cells in cultures transfected with either PAX5 or BACH2-targeting RNPs were observed (mean indel percentages=80% and 86% at the PAX5 and BACH2 target sites, respectively; FIG. 44C, 44D). Concomitant increases in IgM and IgG secretion in PAX5-targeted and an increase in IgM secretion in BACH2-targeted cultures were also observed (FIG. 44E). Again, as predicted, these data indicate that Cas9-mediated disruption of PAX5 or BACH2 enhances human plasma cell differentiation. Further, the IgG/IgM ratio in BACH2-targeted B cell cultures was markedly lower than mock or CCR5-targeted cultures (FIG. 44E, FIG. 45A) and plasmablasts in PAX5-targeted cultures exhibited decreased surface CD19 expression (FIG. 45B), both consistent with previous studies in mice. Taken together, these results demonstrate that CRISPR/Cas9-induced gene disruption in primary B cells is useful for interrogating gene products that may modulate human plasma cell development and function.

### Site-Specific HDR at the PRDM1 Locus Using Co-Delivery of RNP and AAV Donor Template

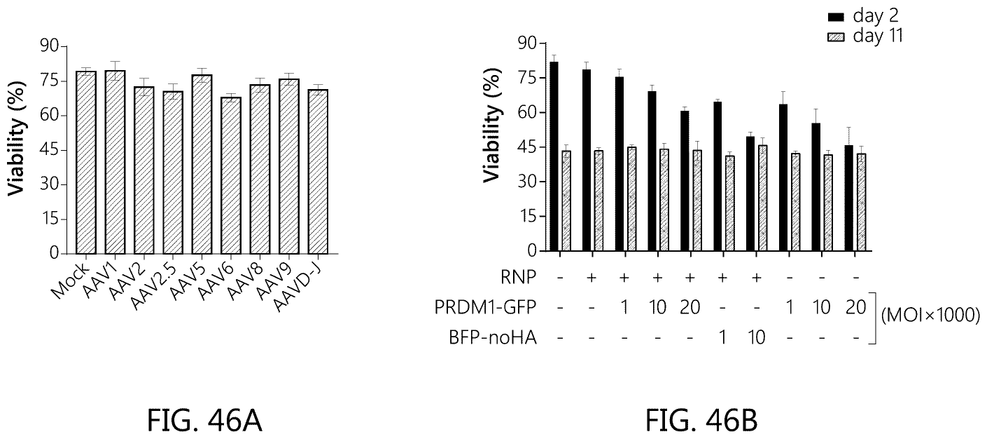

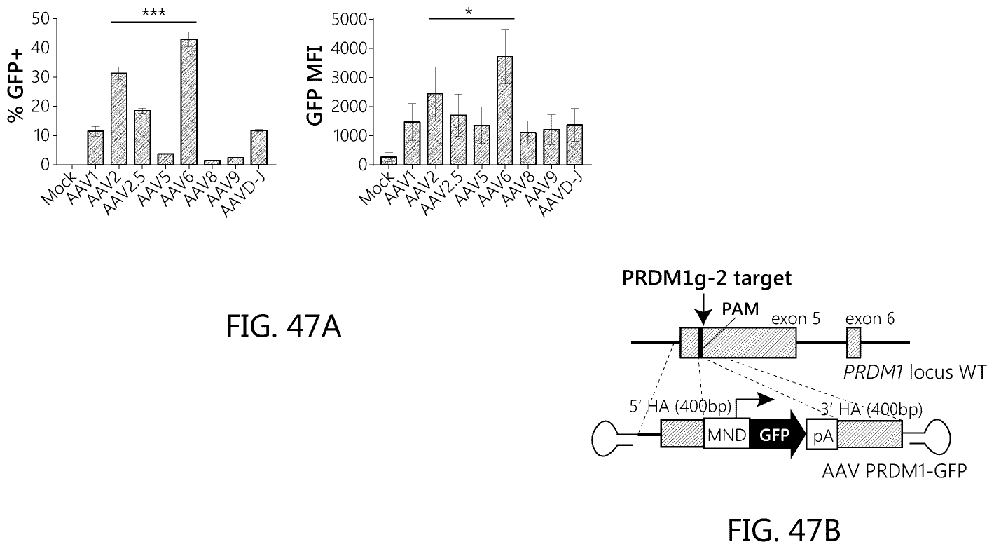

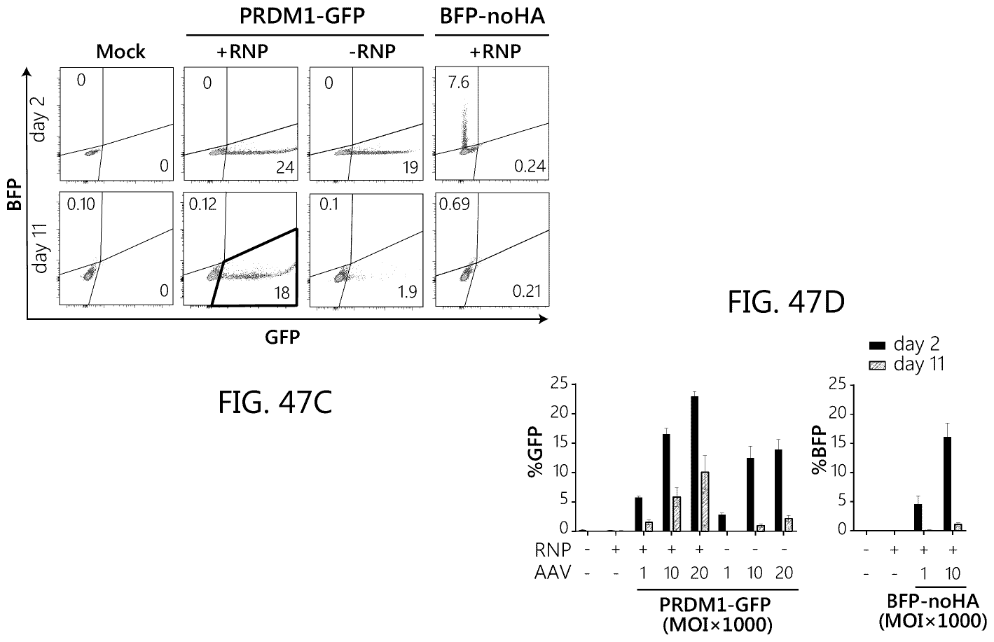

HDR using ssODN donor templates is not suitable for delivering payloads larger than ˜400 bases due to current limitations of the fidelity of ssODN synthesis. In contrast, adeno-associated virus (AAV) can package up to ˜4.7 kilobases (kb) of ssDNA donor template. Many have used AAV to deliver candidate HDR templates leading to high levels of HDR in multiple cell types and at a variety of loci. Of note, there is no published data regarding the capacity of AAV to transduce primary human B cells. To investigate AAV transduction efficiency in human B cells, a self-complementary AAV (scAAV) was designed with a green fluorescent protein (GFP) coding sequence driven by MND, a robust retroviral-derived ubiquitous promoter. Activated B cells was transduced with this vector packaged using various serotypes and quantified GFP expression two days post transduction by flow cytometry. Regardless of serotype, it was observed minimal loss of cell viability following viral exposure (FIG. 46A). It was observed the highest percentage of GFP+ cells (mean=43%) and highest mean fluorescence intensity in B cells transduced with AAV serotype 6 (FIG. 47A).

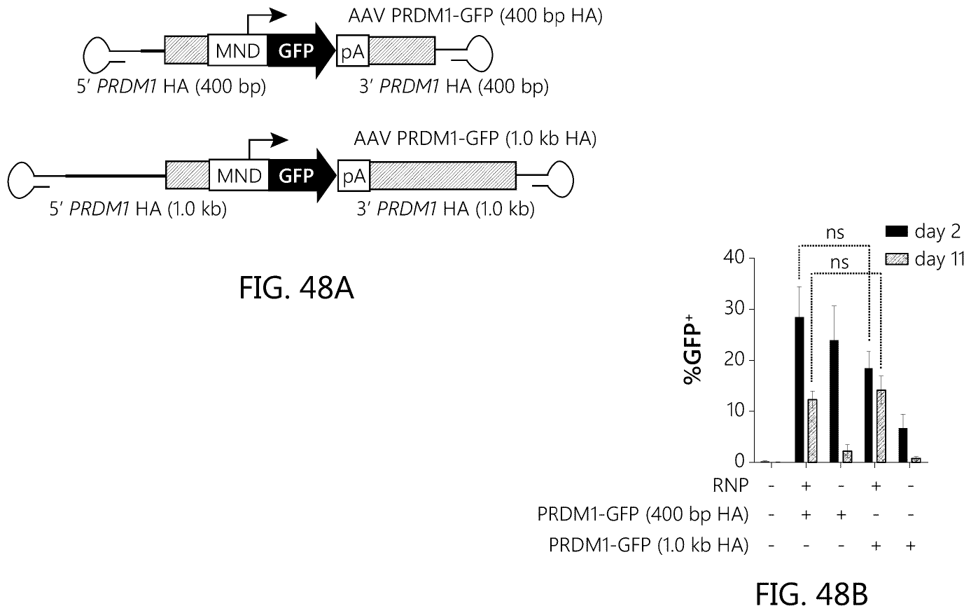

Because the packaging size of single-stranded AAV (ssAAV) is greater than scAAV, the efficacy of HDR-mediated integration of larger payloads was next assessed by delivering ssAAV into B cells. Initially, an AAV6 donor template containing an MND-driven GFP cassette flanked by 400 bp homology arms at the PRDM1g-2 target site was designed (PRDM1-GFP; FIG. 47B). The PAM was not included in the repair template, thereby rendering the repaired sequence non-cleavable by Cas9 (FIG. 47B). After activation and electroporation with or without Cas9 RNP, B cells were transduced with AAV6 PRDM1-GFP at various MOI and cultured for 11 additional days with the same activating factors. Although it was initially observed a 30% viability loss at the highest AAV dose, these cultures eventually recovered and exhibited comparable viabilities by day 11 (FIG. 46B). At the highest AAV MOI, it was observed that there was persistent GFP expression in ˜10% of cells that received both the PRDM1-targeting RNP and AAV repair template, while 2% of cells that were treated with the AAV alone had persistent GFP expression (FIG. 47C). In addition, cells were co-treated with the PRDM1-targeting RNP and a non-targeting AAV BFP control (MND-BFP without homology arms; BFP-noHA) and observed ˜1% BFP expression at the endpoint (FIG. 47C), indicating that the majority of GFP expression resulting from co-delivery of PRDM1-targeting RNP and PRDM1-GFP was likely driven by HDR-mediated integration. It was also found that higher AAV doses correlated with increased percentages of GFP+ cells. Finally, it was found that increasing homology arm lengths from 400 bp to 1.0 kb did not lead to higher levels of HDR (measured as persistent GFP expression; FIG. 48A-48B). Together, these data support the conclusion that co-delivery of Cas9 RNP and AAV can lead to efficient, targeted genomic integration of transgene, likely via the HDR pathway.

Introduction of a BAFF Expression Cassette into CCR5 Leads to Secretion of Functionally Active BAFF

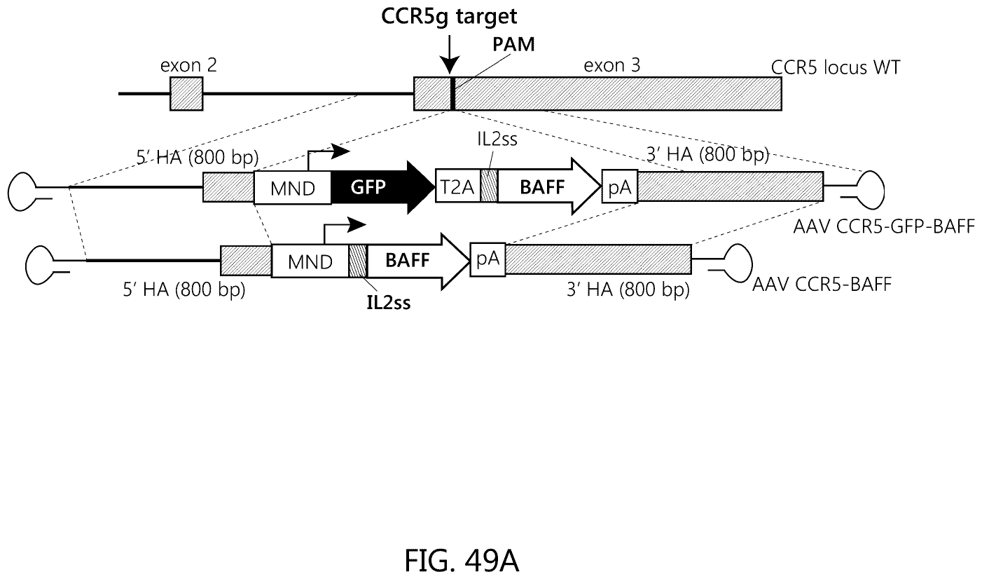

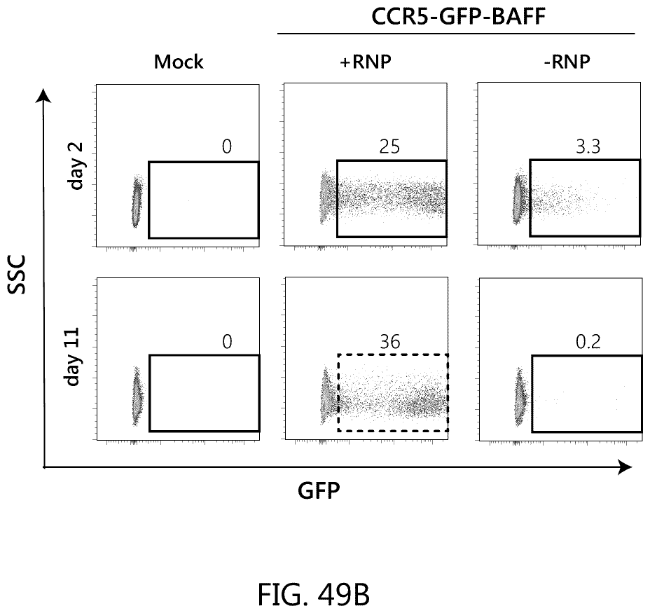

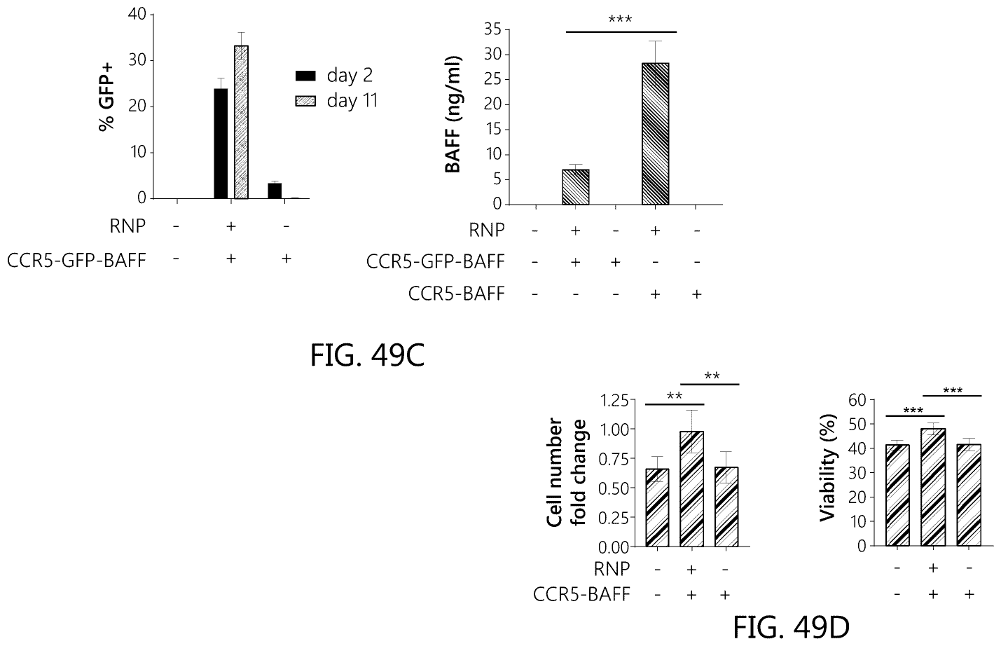

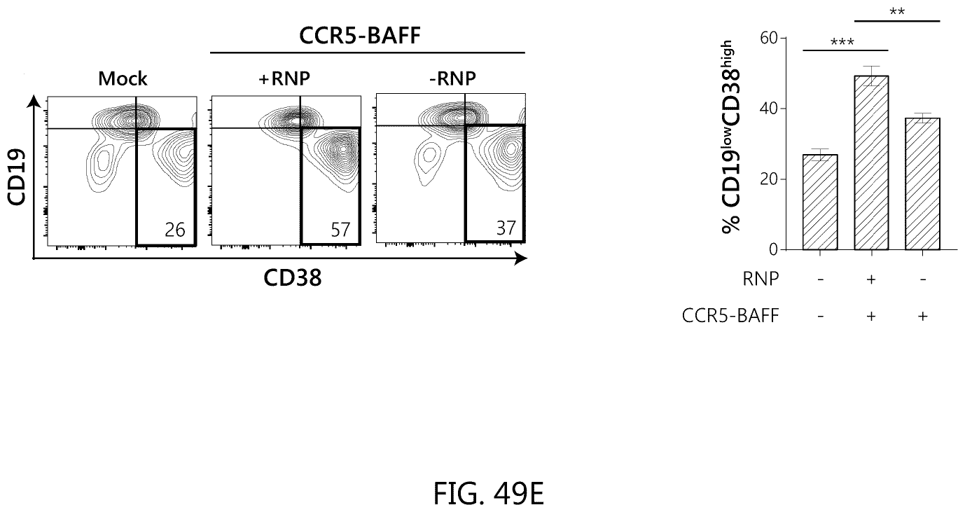

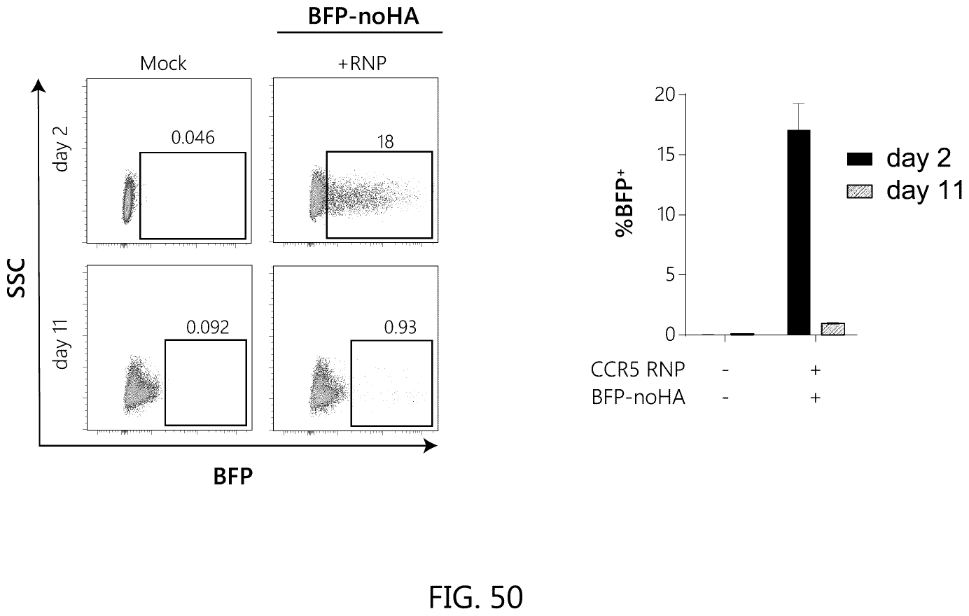

It was next assessed whether primary B cells could be engineered to secrete active proteins with predicted functionality. As a first test of this approach, HDR-mediated knock-in strategy to engineer de novo expression of the B-cell activating factor (BAFF) was used. Of note, the HDR studies targeting the PRDM1 locus (described above) were anticipated to underrepresent HDR efficiencies due to the negative impact of PRDM1 disruption on plasma B cell differentiation and the relative inefficiency of the guide used in the studies (PRDMlg-2; FIG. 42A). Therefore, the CCR5 locus was targeted as a “safe harbor” because: (a) it is not transcriptionally active in human B cells; (b) it is not required for plasma cell differentiation (FIG. 44A-44D); and (c) heterozygous and homozygous null mutations in CCR5 are innocuous to human carriers. Two AAV6 repair templates were designed: one contained MND-driven GFP and BAFF linked with a T2A self-cleaving peptide (CCR5-GFP-BAFF) and the other contained only an MND-BAFF expression cassette (CCR5-BAFF). Both donor constructs contained 800 bp homology arms around the CCR5 guide target site (FIG. 49A). Following RNP transfection and/or AAV transduction, cells were cultured using the three-step plasma cell culture system (FIG. 44A). As in earlier PRDM1-targeting experiments, persistent GFP expression was observed (mean=33% GFP⁺) only in the condition containing both CCR5-targeting RNP and AAV (FIG. 49B, FIG. 50). While use of both donor templates led to BAFF secretion, B cells targeted by CCR5-BAFF (repair template solely containing BAFF) exhibited much higher levels of BAFF secretion (4.1 fold increase) relative to those targeted by CCR5-GFP-BAFF (template containing BAFF downstream of the T2A linkage) (FIG. 49C) To determine if this exogenous BAFF is functional, cell expansion and plasmablast formation was assessed following genome editing and in vitro differentiation. In the CCR5-BAFF edited culture that contained the highest concentration of exogenous BAFF, increases in cell number was observed, viability and percentage of CD19^{low}CD38^{high} plasmablasts relative to controls that lacked Cas9 RNP (FIG. 49D-49E). This is consistent with previous studies showing that BAFF promotes both human and murine B cell survival and differentiation. Taken together, these results show that Cas9 RNP- and AAV-mediated genome editing of primary human B cells can be used to generate functional, protein-secreting human plasma cells.

### Generation of FIX-Secreting Human Plasma Cells Via HDR-Mediated Integration at the CCR5 Locus

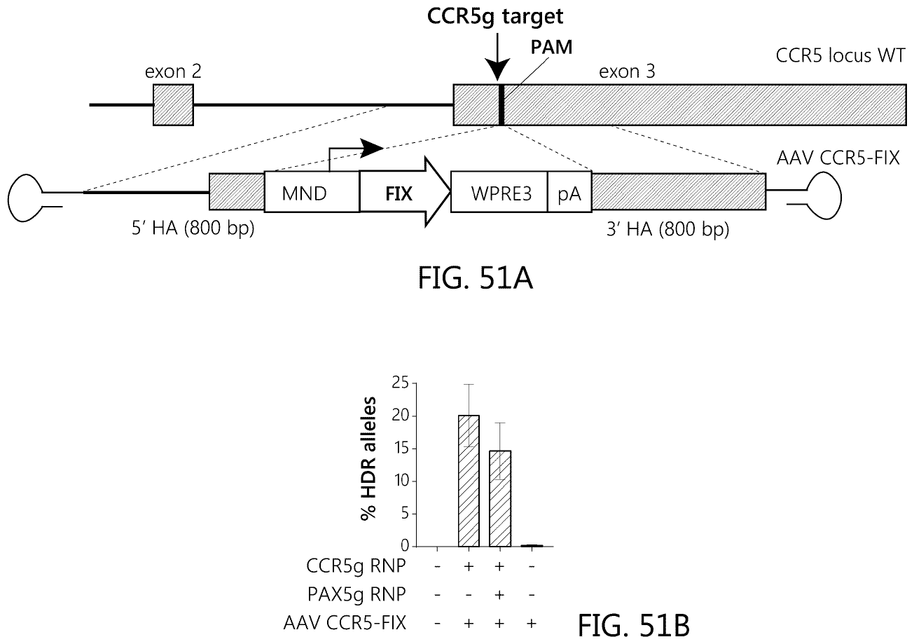

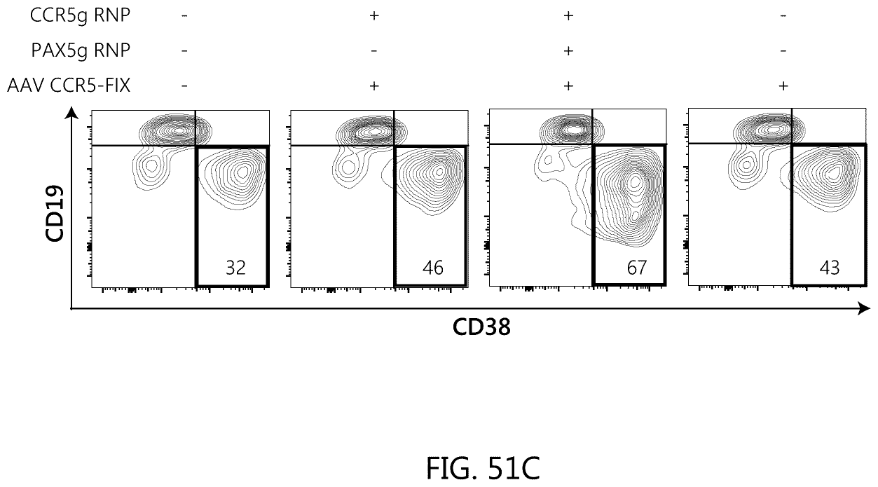

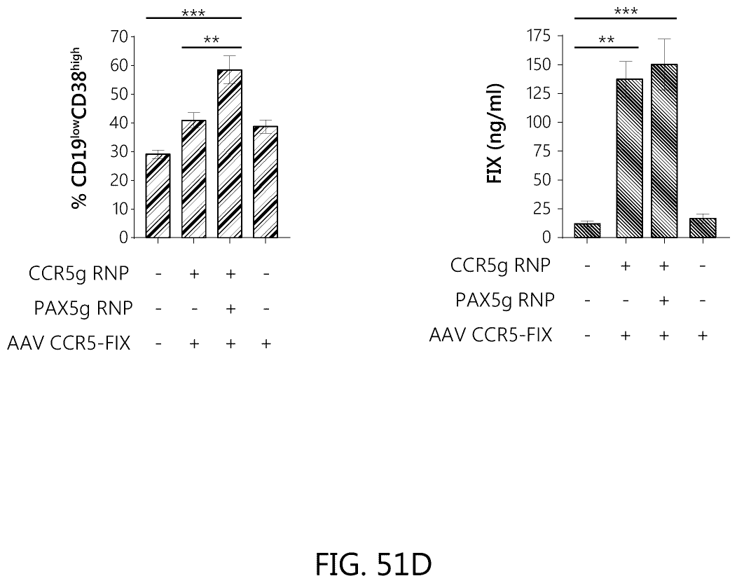

Engineering exogenous protein production in plasma cells may have therapeutic applications in protein deficiency diseases such as hemophilia B, which is caused by a deficiency of FIX. To generate plasma B cells capable of secreting exogenous human FIX, an AAV vector containing an MND-driven FIX (codon-optimized FIX-R338L Padua variant) expression cassette was co-delivered, with the same flanking CCR5 homology arms as described above (CCR5-FIX), into primary human B cells with or without CCR5-targeting RNP (FIG. 51A). To also boost plasma cell differentiation in these cultures, PAX5-targeting RNPs were also co-delivered in a subset of CCR5-targeted cells. Although similar rates of HDR at the CCR5 locus across conditions were observed (FIG. 51B), following plasma cell differentiation increases in CD19^{low}CD38^{high} plasmablast percentages in the cultures that received CCR5- and PAX5-targeting RNPs were observed (FIG. 51C With or without delivery of PAX5-targeting RNPs, high levels of de novo FIX secretion in the cultures were detected (FIG. 51D). In addition, HDR cultures co-treated with both CCR5- and PAX5-targeting RNPs exhibited similar FIX production in dually-edited cell populations (FIG. 51D). These data demonstrate FIX production via targeted HDR in gene-modified human B cells and the use of multi-locus modifications to simultaneously drive plasma cell differentiation and de novo protein secretion.

### BAFF-Secreting Human Plasma Cells Exhibited High Secretory Capabilities in NSG Mice

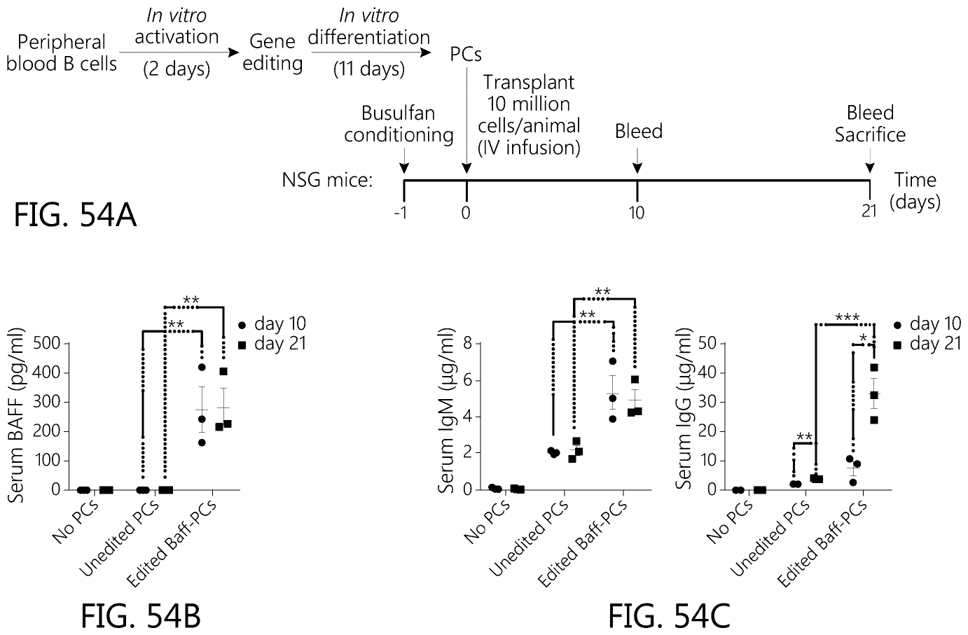

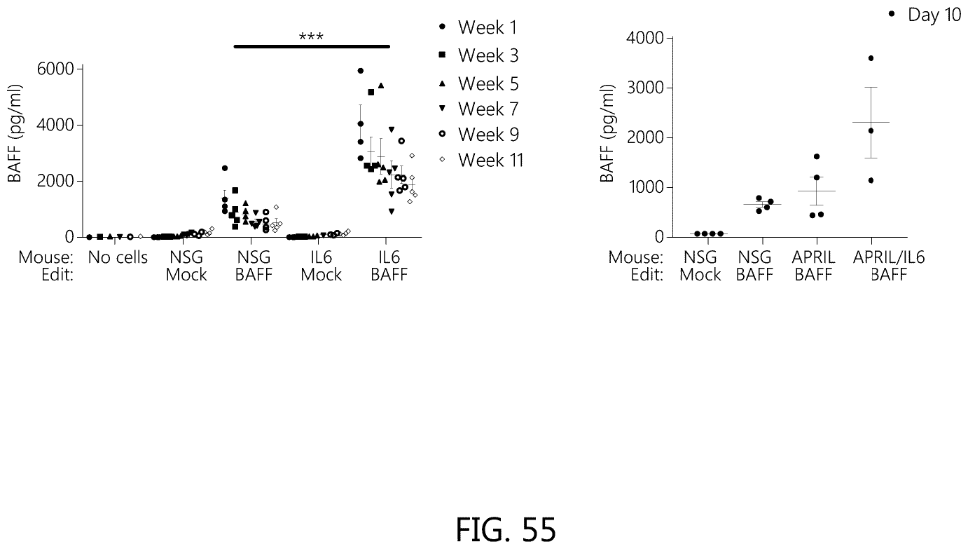

It was then tested whether autocrine BAFF secretion can promote plasma cell survival in humanized mice. Gene-edited B cells were first generated using the CCR5-targeting RNP and the CCR5-GFP-BAFF AAV donor template. These cells were differentiated into plasma cells in vitro using the three-step culture system as before and subsequently transplanted into immuno-deficient NOD/SCID/gamma-c null (NSG) mice via intravenous infusion (FIG. 54A). Equal numbers of unedited plasma cells were also transplanted into parallel recipient mice as controls. Blood samples were collected at days 10 and 21 and serum human proteins were quantified by ELISA (FIG. 54A). As expected, human BAFF was detected in mice that received BAFF-expressing plasma cells, but not in mice that received unedited plasma cells (FIG. 54B). Also was predicted, human IgM and IgG were detected only in mice that received plasma cells but not in the untreated mice (FIG. 54C). It was also found that serum BAFF and IgM levels remained stable from day 10 to day 21, while IgG levels significantly increased in this period (FIGS. 54B, 54C). Most strikingly, significantly higher levels of serum IgM and IgG were observed in mice that received BAFF-expressing plasma cells compared to mice that received unedited plasma cells (FIG. 54C; difference in IgG levels at day 10 was not significant). These findings demonstrate that gene-modified plasma cells maintain stable secretory capacities in an immune-compromised murine setting for at least 3 weeks and support the hypothesis that expression of a survival factor such as BAFF via genome editing promotes plasma cell secretory function and/or survival in vivo.

As described, high rates of gene disruption in primary human B cells at multiple loci using CRISPR/Cas9 RNPs were accomplished. This method is applied to modeling the impact of genetic changes on human plasma B cell differentiation. It also demonstrates the use of Cas9 RNP in combination with ssODN or AAV6 repair templates to achieve high-efficiency HDR in B cells and to engineer secretion of functional and therapeutically relevant proteins. Importantly, gene-modified cells obtained a plasma cell phenotype and remained viable for several weeks in culture, providing evidence of utility for adoptive cell therapies using engineered human plasma cells.

Cas9-mediated disruption of PRDM1, IRF4, PAX5, orBACH2, which either promote (PRDM1, IRF4) or inhibit (PAX5, BACH2) plasma cell differentiation in mice, led to human B cell phenotypes consistent with these regulatory roles. The ability to easily disrupt genes or induce single-base changes using Cas9 RNP with or without ssODN-mediated HDR will facilitate further study of the impact of genetic changes on human B cell development. When combined with differentiation in vitro, this system is useful as a high-throughput model for studying genetic mutations that alter human B cell development, including somatic and/or germ-line mutations associated with a range of human B cell disorders (in B cell lymphoma and in autoimmune disorders including systemic lupus erythematosus); as well as a range of mechanistic studies designed to assess candidate genes identified by genome-wide association studies.

The results of the alternative methods described herein show that Cas9 RNP and ssODN or AAV co-delivery into B cells leads to high levels of HDR with minimal toxicity. Compared to double-stranded DNA transfection, which promotes apoptosis in primary B and T cells, single-stranded DNA delivery, either as ssODN or recombinant AAV, is relatively non-toxic to primary B cells. It is hypothesized that both approaches may be protected from cyclic GMP-AMP synthase (cGAS)-dependent type I interferon responses prevalent in primary B cells. Consistent with this, compared with dsDNA, ssDNA exhibits significantly lower binding affinity for the cytosolic DNA sensor cGAS.

The development of an alternative method for the efficient, site-specific introduction of transgenes via HDR opens the possibility of engineering plasma cells to act as autologous “cell factories,” capable of delivering sustained, high doses of therapeutic proteins to subjects. As a proof of concept, plasma cells were engineered to secrete FIX. Deficiency of FIX is the cause of hemophilia B, a genetic disease characterized by blood clotting defects. Current treatment for hemophilia B is limited to protein replacement therapy, which is costly and non-curative. Recent gene therapy trials reported long-term FIX expression in patients after intravenous injection of a liver-tropic AAV8-FIX vector; however, pre-existing AAV neutralizing antibodies to all known AAV serotypes are prevalent in humans and greatly limit in vivo AAV transduction, making a significant group of patients with anti-AAV antibodies ineligible for treatment. In addition, systemic AAV delivery induces humoral immunity against AAV that prevents subsequent delivery, thus limiting AAV therapy to a single dose and/or necessitating use of alternative serotypes. Delivery of ex vivo gene-edited plasma cells producing FIX would remove the requirement for systemic AAV delivery and likely avoid limitations posed by AAV neutralizing antibodies.

In addition, immune responses to therapeutic proteins comprise a key unaddressed challenge. Based on evidence that B cell delivery can induce tolerance, an additional application of B cell editing technology is to deliver smaller numbers of short-lived B cells producing FIX, or other therapeutic proteins, with the goal of inducing tolerance to the secreted product.

The experiments above demonstrate high rates of locus-specific genome editing in primary human B cells. These engineered B cells retain the ability to differentiate into plasma cells ex vivo and secrete physiological doses of therapeutic proteins such as FIX. The ability to efficiently target specific loci enables us to drive B cell differentiation and secretory programs. Thus, creative multiplexing of gene disruption and HDR will provide ever more powerful tools to coordinately optimize protein secretion, cellular phenotype and long-term survival.

CRISPR/Cas9 Reagents and ssODNs

CRISPR guide targets at the CCR5, PRDM1, 1RF4, PAX5, BACH2 loci were identified using an online MIT CRISPR design tool and the Broad Institute sgRNA design tool. A tracrRNA recognition sequence (5′ GUUUUAGAGCUAUGCU 3′ (SEQ ID NO: 1)) was added to the 3′ end of each selected guide target to form the complete synthetic crRNA sequence. As shown in Table 2 below are guide sequences used in the alternative herein:

TABLE 2
Guide RNA sequences for CCR5, PRDM1, IRF4, PAX5,
and BACH2. Each synthesized crRNA comprises a
protospacer immediately followed by a tracrRNA
binding sequence.

| Guide | | tracrRNA binding |
|---|---|---|
| designation | Protospacer | sequence |
| CCR5g | CAAUGUGUCAACUCUUGACA | GUUUUAGAGCUAUGCU |
| PRDM1g-1 | AGGATGCGGATATGACTCTG | |
| PRDM1g-2 | GGGGAGCGAGTGATGTACGT | |
| IRF4g | CAAGCAGGACTACAACCGCG | |
| PAX5g | UGUGAAUGGACGGCCACUCC | |
| BACH2g | GUUCCUGCGCAUGCACAACC | |

As shown in the Table 2 above are the sequences for CCR5G (SEQ ID NO: 20), PRDM1g-1 (SEQ ID NO: 21), PRDM1g-2 (SEQ ID NO: 22), IRF4g (SEQ ID NO: 23), PAX5G (SEQ ID NO: 24), BACH2g (SEQ ID NO: 25) and the tracrRNA binding sequence (SEQ ID NO: 26). The crRNA guides were synthesized by IDT® with additional modifications: phosphorothioate linkages between the four nucleotides on each end, as well as 2′O-methyl groups on the three nucleotides on each end. The tracrRNA with proprietary chemical modifications, as well as the recombinant Cas9 nuclease were also purchased from IDT®. In some alternatives, the guide sequence comprises a sequence set forth in any one of SEQ ID NO: 2-13, 20-25 or 63-112.

Prior to delivery of the nuclease into cells, the crRNA and tracrRNA were mixed at an equimolar ratio. The mixture was heat-shocked at 95° C. for 5 minutes and then incubated at room temperature for 30-60 minutes to allow crRNA: tracrRNA hybrids to form. Next, the crRNA:tracrRNA hybrids were mixed with the Cas9 nuclease at a 1.2:1 molar ratio and incubated at room temperature for 10-20 minutes to allow Cas9:crRNA:tracrRNA complexes to form. The Cas9 ribonucleoprotein (RNP) complexes were then delivered into cells by electroporation.

Single-stranded oligonucleotides (ssODNs) were commercially synthesized by IDT® (Ultramer® DNA Oligonucleotides) with phosphorothioate linkages between the three nucleotides on each end.

### Production of Recombinant AAV Vectors

The self-complementary AAV GFP (scAAV GFP) construct contains an MND promoter, enhanced green fluorescence protein (eGFP) and an a globin polyadenylation (pA) signal in an scAAV plasmid backbone. In contrast, all AAV donor templates designed for HDR experiments were cloned into single-stranded AAV plasmid backbones. PRDM1-GFP (400 bp homology arms) contains an MND promoter followed by eGFP and an SV40 pA signal. This MND-eGFP-SV40 pA cassette is flanked by two 400 bp sequences homologous to PRDM1, with the 5′ arm spanning from 106,104,721 to 106,105,120 and the 3′ arm spanning from 106,105,124 to 106,105,523 on chromosome 6, with reference to the UCSC December 2013 human genome assembly (GRCh38/hg38). PRDM1-GFP (1 kb homology arms) contains an identical MND-eGFP-SV40 pA cassette that is flanked by two 1 kb sequences homologous to PRDM1, with the 5′ arm spanning from 106,104,121 to 106,105,120 and the 3′ arm spanning from 106,105,124 to 106,106,123 on chromosome 6 (GRCh38/hg38). BFP-noHA contains MND promoter-driven blue fluorescent protein (mTagBFP; Evrogen) and an SV40 pA tail without genomic homology. CCR5-GFP-BAFF comprises an MND promoter-driven eGFP and T2A cleavage peptide linking a BAFF coding sequence, which is preceded by an IL2 signal sequence (IL2ss), and an SV40 pA tail. This cassette is flanked by a 823 bp 5′ CCR5 homology arm, spanning from 46,372,387 to 46,373,209, and a 804 bp 3′ CCR5 homology arm, spanning from 46,373,221 to 46,374,024 on chromosome 3 (GRCh38/hg38). CCR5-BAFF comprises identical CCR5 homology arms flanking a similar cassette, to the exclusion of eGFP and the T2A peptide (i.e. BAFF directly under the MND promoter). Finally, CCR5-FIX substitutes a FIX coding sequence and a succeeding modified woodchuck hepatitis virus posttranscriptional regulatory element (WPRE3) for the IL2ss and BAFF coding sequence in CCR5-BAFF.

AAV stocks were produced as known in the art. The AAV vector, serotype helper and HgT1-adeno helper plasmids were transfected into HEK293T cells. Cells were harvested 48 hours later, lysed by 3 freeze-thaw cycles, and the cell lysate was treated with benzonase. Virions with recombinant AAV genomes were purified using an iodixanol density gradient.

All multiplicity of infection (MOI) calculations were based on qPCR-based titers of AAV genomes using ITR specific primers and probe.

### Primary Human CD19+ B Cell 2Enome Editing

Peripheral blood mononuclear cells (PBMCs) were collected from whole blood of consented donors and cryopreserved. After thaw, PBMCs were treated with 1X ACK buffer; CD19+ B cells were subsequently isolated from PBMCs by negative selection using a human B cell isolation kit (Miltenyi Biotec®, Auburn, CA) and cultured in Iscove's modified Dulbecco's medium (IMDM; Thermo Fisher Scientific®) supplemented with 10% fetal bovine serum and 55 M beta-mercaptoethanol at 1-1.5×10⁶ cells/ml. B cells were activated with 100 ng/ml of recombinant human MEGACD40L® (Enzo Life Sciences®), 1 μg/ml of CpG oligodeoxynucleotide 2006 (Invitrogen®), 50 ng/ml of IL2 (Peprotech®), 50 ng/ml of IL10 (Peprotech®) and 10 ng/ml of IL15 (Peprotech®) for two days. Cells were then electroporated with Cas9 RNP complexes using the Neon Transfection System (ThermoFisher Scientific®) as follows. Cells were washed with PBS and resuspended in Neon Buffer T. 30 pmol Cas9 RNP per 3×10⁵ cells was added to the resuspension so that the final cell density was 3×10⁷ cells/ml. Cells were electroporated (1700 V, 20 ms, 1 pulse) in 10-1 Neon tips, and then transferred into pre-warmed B cell culture medium with MEGACD40L®, CpG, IL2, IL10 and IL15 and cultured at 1.5×10⁶ cells/ml. For samples transfected with an ssODN donor template, ssODN was transfected concurrently with Cas9 RNP at the specified amount. For samples transduced with AAV, AAV was added to the culture immediately after electroporation at MOIs ranging from 10,000 to 100,000. Culture volume was doubled 24 hours after electroporation, and medium was replenished every two to three days thereafter to maintain a cell density of 1×10⁶ cells/ml. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days.

### Flow Cytometry

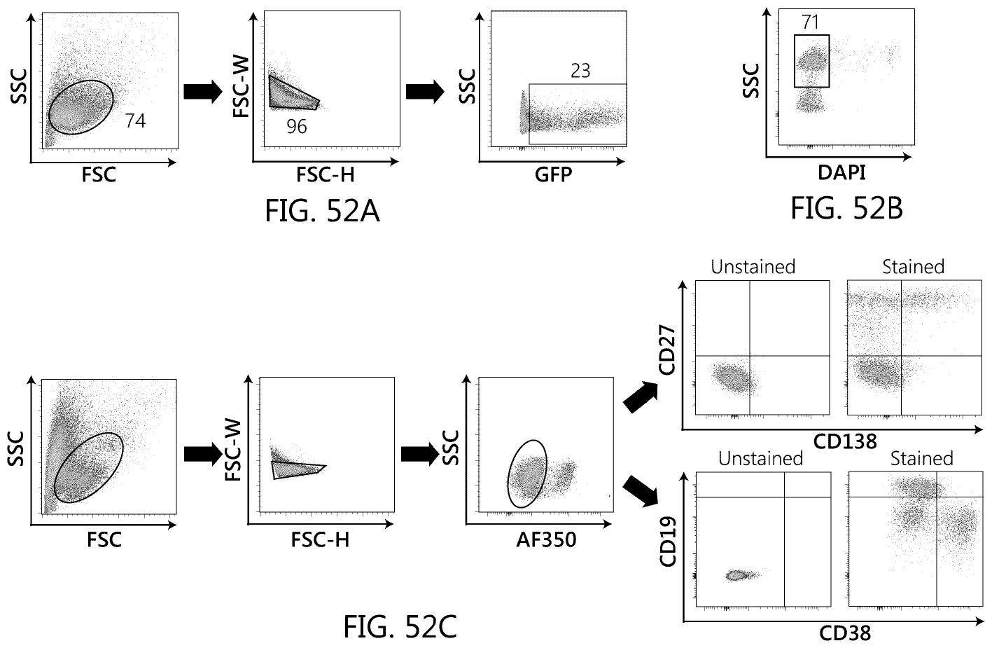

Flow cytometric analysis was done on an LSR II flow cytometer (BD Biosciences®) and data were analyzed using FlowJo software (TreeStar). To assess fluorochrome expression in B cells, flow gates were drawn on FSC/SSC populations corresponding to live cell sizes and singlets were defined using FSC-W/FSC-H gates; BFP+ or GFP⁺ gates were then used to define fluorescent cells (FIG. 52A). Viabilities of these cells were also confirmed by staining them separately with 4,6-Diamidino-2-phenylindole (DAPI) and subsequently quantifying percentages of DAPI-live cells by flow cytometry (FIG. 52B). To assess B cell surface marker expressions, cells were stained with fluorophore-conjugated antibodies: CD19-PECy7 (clone HIB19, eBioscience®), CD27-APC (clone 0323, Life Technologies®), CD38-PerCPCy5.5 (clone HIT2, BD Biosciences®) or CD38-FITC (clone T16, Beckman Coulter®), CD138-Alexa Fluor 700 (clone MIl5, BioLegend®), IgD-PE (clone IA6-2, BD Biosciences®), IgM-Pacific Blue (clone MHM-88, BioLegend®). Dead cells were excluded using Fixable Live/Dead stain-Alexa Fluor 350 (LifeTechnologies®) (FIG. 52C for the general gating strategy used for analyzing B cell immunophenotypes).

### Plasma Cell Differentiation Culture

Plasma cells were differentiated in vitro using a three-step culture system as known in the art. CD19+ B cells isolated from PBMCs were activated for 2 days with MEGACD40L® (100 ng/ml), CpG (1 μg/ml), IL2 (50 ng/ml), IL10 (50 ng/ml) and IL15 (10 ng/ml) and gene-edited as outlined above. Cells were then expanded for another 5 days in the same cocktail. Next, cells were washed with 1×PBS and seeded in medium with IL2 (50 ng/ml), IL6 (50 ng/ml), IL10 (50 ng/ml) and IL15 (10 ng/ml) for 3 days. At day 8 after gene-editing, cells were washed with 1×PBS and seeded in medium with IL6 (50 ng/ml), IL15 (10 ng/ml) and human interferon-α 2B {100 U/ml, Sigma-Aldrich®) for 3 days to stimulate plasma cell differentiation. At day 11 after gene-editing, cell phenotypes were analyzed by immunofluorescence.

In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days.

### Molecular Analyses of Gene Disruption and Single-Nucleotide HDR

Total genomic DNA was isolated from 0.2-1.0×10⁶ cells using a DNeasy Blood & Tissue Kit (Qiagen). To assess gene disruption using the T7 endonuclease 1 assay, guide target genomic regions were first amplified using either AccuPrime™ Pfx DNA Polymerase (ThermoFisher Scientific®) or PrimeSTAR® GXL DNA Polymerase (Takara Bio) with primers about 250-350 bp away from the guide target site (See table 3 below).

TABLE 3
Primers used for molecular analysis of gene-edited B cells.

| | Genomic | | | |
|---|---|---|---|---|
| Assay | target | Forward primer | Reverse primer | Probe |
| T7E1 assay | CCR5g | ATGTGAAGCAAATCGCAGCC | TCCCGAGTAGCAGATGACCA | |
| | | (SEQ ID NO: 27) | (SEQ ID NO: 30) | |
| | PRDM1g-1 | TAGCATTTAAAAACCTTGCTTCTTTTCAAG | TACAGATTCTCAGAGGTTTTCAGAGA | |
| | | (SEQ ID NO: 28) | (SEQ ID NO: 31) | |
| | PRDM1g-2 | CAGTCTAAAGCAACCGAGCAC | CTTGGGGTAGTGAGCGTTGTA | |
| | | (SEQ ID NO: 29) | (SEQ ID NO: 32) | |
| Illumina | CCR5g | GGCAACATGCTGGTCATCCT | GGTGACCGTCCTGGCTTTTA | |
| sequencing | | (SEQ ID NO: 33) | (SEQ ID NO: 39) | |
| | PRDM1g-1 | CTCTCAGAAGGAGCCACAGG | TTGGTGGCATACTTGAAAAGC | |
| | | (SEQ ID NO: 34) | (SEQ ID NO: 40) | |
| | PRDM1g-2 | CCCCTCACATCAGAAAAGGA | CTGGAGCTCTTGAGGCTTTG | |
| | | (SEQ ID NO: 35) | (SEQ ID NO: 41) | |
| | IRF4g | AGATCGACAGCGGCAAGTA | GAGGCCTCCTTTCCTCCTC | |
| | | (SEQ ID NO: 36) | (SEQ ID NO: 42) | |
| | PAX5g | CACAGCGGTGCTTCTCCTAT | GCCTCGAGCTACTGCCTTTA | |
| | | (SEQ ID NO: 37) | (SEQ ID NO: 43) | |
| | BACH2g | TGAGGGATTCGGGACAATAG | AGTTCTCGCAGTCCTCGTGT | |
| | | (SEQ ID NO: 38) | (SEQ ID NO: 44) | |
| In-out | CCR5 (HDR) | GGTATGATGCTTAGAACAGTGAT | CCATATTCTGCTGTTCCAACG | CCTGGGCAACATAGTGTGATC |
| | | (SEQ ID NO: 45) | (SEQ ID NO: 47) | (SEQ ID NO: 49) |
| ddPCR | ActB | ACTCTGCAGGTTCTATTTGC | AATGATCTGAGGAGGGAAGG | ATCAAGGTGGGTGTCTTTCC |
| | | (SEQ ID NO: 46) | (SEQ ID NO: 48) | (SEQ ID NO: 50) |

As shown in Table 3 above, the forward primers for CCR5g, PRDM1g-1, and PRDM1g-2 for the T7E1 assay are SEQ ID NO: 27, 28 and 29, respectively. The reverse primers for CCR5g, PRDM1g-1, and PRDM1g-2 for the T7E1 assay are SEQ ID NO: 30, 31and 32 respectively. As shown in table 3, the forward primers for CCR5g, PRDM1g-1, PRDM1g-2, IRF4g, PAX5g, and BACH2g are SEQ ID NO: 33, 34, 35, 36, 37 and 38 respectively, for the sequencing in the Illumina sequencing. The reverse primers for CCR5g, PRDM1g-1, PRDM1g-2, IRF4g, PAX5g, and BACH2g are SEQ ID NO: 39, 40, 41, 42, 43 and 44, respectively, for in the Illumina sequencing The forward primers for CCR5 (HDR) and ActB are SEQ ID NO: 45 and 46, respectively, for the in-out ddPCR. The reverse primers for CCR5 (HDR) and ActB are SEQ ID NO: 47 and 48, respectively, for the in-out ddPCR. The probe for CCR5 comprises SEQ ID NO: 49 and the probe for ActC comprises SEQ ID NO: 50.

PCR products were then purified using NucleoSpin Gel and PCR Clean-up kit (Macherey-Nagel®). 200 ng purified PCR product were denatured and re-annealed in 1×NEB Buffer 2 (New England Biolabs®, Ipswich, MA) in 19 μl total volume, after which 10 U of T7 endonuclease I (New England Biolabs®) was added to the re-annealed PCR product and the samples were incubated for 1 hour at 37° C. The reactions were immediately run on an agarose gel for imaging.

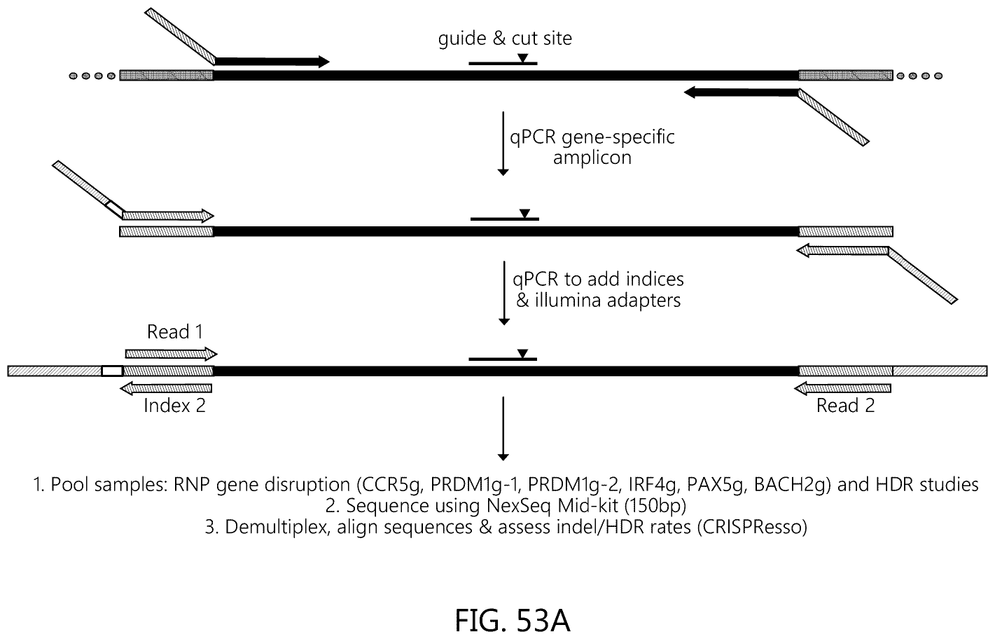

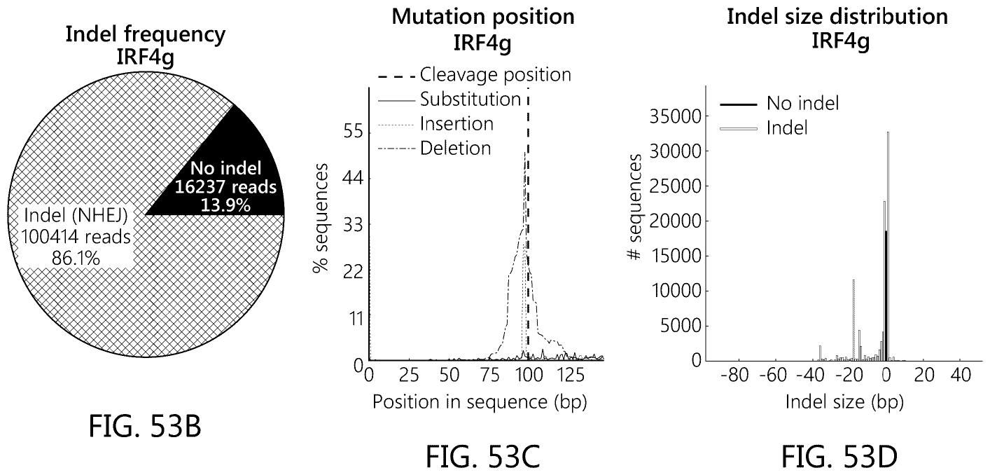

To quantify frequencies of on-target indels or nucleotide substitutions, allelic modifications by sequencing was assessed (FIG. 53A-53D). Locus-specific primers were first designed (Table 3) within 150 bp from each guide target site, each primer also containing an adaptor sequence on the 5′ end. To prepare samples for sequencing, amplification was performed each using qPCR starting with 250 ng of genomic DNA using PrimeSTAR® GXL Polymerase with SYBR Green I Nucleic Acid Gel Stain until reaction reached the exponential phase. The samples were purified with Agencourt® AMPure® XP and amplified using qPCR for another 6 cycles with primers that contain unique index sequences for each experimental condition, as well as the p5 and p7 cluster generating sequences. The primers were removed using Agencourt® Ampure® XP and purity of the amplicon was confirmed using a 4-12% Novex® TBE PAGE Gel. Next, amplicons from each locus and experiment were pooled at equimolar concentrations and sequenced on a NextSeq® 500 in paired-end run mode with a NextSeq® 500/550 Mid Output Kit v2. Reads were filtered for quality and grouped by sample index. Next, 112,000 reads per condition and experiment were aligned to the wild type sequences of each amplicon using the CRISPResso⁵⁷ aligner and analysis suite (the aligner runs a variant of the Needleman-Wunsch algorithm). The minimum average read quality score (phred33) was set to 30 and indels/substitutions were specifically quantified within a 30 bp window around each guide target.

To calculate editing rates at the CCR5 locus, “in-out” ddPCR was performed with forward oligo binding the CCR5 locus outside the homology region and reverse within the AAV insert. A similar size amplicon (1.3 kb) was generated for ActB gene to serve as a control. Probes for both amplicons were labeled with FAM and reactions for each were performed in different wells. The oligo and probe sequences are included in Table 3. The PCR reactions were partitioned into droplets using droplet-generating oil for probes on a QX200 Droplet Generator. Amplification was performed using ddPCR Supermix for Probes without UTP (Bio-Rad®), 900 nM of primers and 250 nM of Probe. 50 ng of genomic DNA was used in a 25 μl amplification reaction containing 1% DMSO and run according to manufacturer's directions. The droplets were scanned using the QX200 Droplet Digital PCR System (Bio-Rad®) and analyzed using QuantaSoft software. The editing rates were calculated as a ratio of the copies/μl from CCR5/ActB positive droplets.

### ELISA

3 days prior to measuring culture protein levels, cells were collected, washed with PBS and then resuspended in new culture medium at 1×10⁶ cells/ml. After 3 days, the culture supernatant was collected and Ig or recombinant protein secretion levels were determined by enzyme-linked immunosorbent assay (ELISA). IgG and IgM concentrations were measured using Human IgG total ELISA Ready-SET-Go® and Human IgM total ELISA Ready-SET-Go® kits (eBioscience®, San Diego, CA). BAFF concentrations were measured using a Human BAFF/BLyS/Quantikine ELISA Kit (R&D Systems®, Minneapolis, MN), and FIX concentrations were measured using a FIX Human ELISA Kit (Abcam®, Cambridge, MA).

### Western Blot

TMD8 cells were mock treated or transfected with 30 pmol of either of the PRDM1-targeting RNPs. After five additional days of culture, cells were lysed in RIPA and the lysate was run on a NuPAGE® 4-12% Bis-Tris protein gel. Cell Signaling Blimp-1/PRDI-BF1 Rabbit mAb #9115 was used as the primary antibody for the PRDM1 locus and Licor IRDye® 800CW Goat anti-Rabbit IgG (H+L) was used as the secondary antibody.

### Statistical Analysis

Statistical analyses were performed using Graphpad Prism® 7 (GraphPad®, San Diego, CA). p-values between two groups were calculated using the unpaired two-tailed t-test, while p values in multiple comparisons were calculated using one-way ANOVA with the Sidak correction as specified. Values from independent experiments are shown as means SEM.

### Data Availability

Accession codes: all sequencing data are accessible at the NCBI Sequence Read Archive through the following study accession number: SRP113557.

### More Alternatives

The ability to engineer primary human B cells to differentiate into long-lived plasma cells and secrete a de novo protein may allow the creation of novel plasma cell therapies for protein deficiency diseases and other clinical applications. Methods for efficient genome editing of primary B cells isolated from peripheral blood were initially developed as described in the alternatives herein. By delivering CRISPR/Cas9 ribonucleoprotein (RNP) complexes under conditions of rapid B cell expansion, site-specific gene disruption at multiple loci in primary human B cells was achieved (with editing rates up to 94%). This method was used to alter ex vivo plasma cell differentiation by disrupting developmental regulatory genes. Next, RNPs were co-delivered with either single-stranded DNA oligonucleotide or adeno-associated viruses containing homologous repair template. Using either delivery method, targeted sequence integration at high efficiency (up to 40%) via homology-directed repair was achieved. This method enabled engineering of plasma cells to secrete factor IX (FIX) or B-cell activating factor (BAFF) at high levels. Finally, as shown in the alternatives herein, introduction of BAFF into plasma cells promotes their engraftment into humanized mice. The results as shown in the alternatives herein, highlight the utility of genome editing in studying human B cell biology and demonstrate a novel strategy for modifying human plasma cells to secrete therapeutic proteins.

Long-lived plasma cells stably reside in the bone marrow for decades and secrete large quantities of antibodies. Consequently, plasma cells engineered to produce de novo proteins have the potential to be curative therapies for protein deficiency diseases, prophylaxis for infectious diseases and many other applications. However, the development of plasma cell therapeutics has been limited by technical challenges in the in vitro modification, culture, expansion and differentiation of primary human B cells. B cells can be transduced at high rates by recombinant adenovirus or Epstein-Barr virus (EBV) vectors, which deliver transgenes as episomes. However, episomal DNA expression is lost over time, limiting use of these vectors in applications that require long-term transgene expression. Unlike non-integrating vectors, gamma retrovirus (γRV) and lentivirus (LV) randomly integrate into the host genome and can be used to introduce stably expressing transgenes. However, these vectors are inefficient at transducing primary human B cells. LV that employ alternative envelopes, including that of baboon retrovirus, measles virus, or gibbon-ape leukemia virus exhibit higher B cell transduction rates (up to ˜50%), but have low viral titers that make large-scale production challenging. Because γRV and LV vectors do not efficiently transduce B cells while transduction by non-integrating vectors results in only transient transgene expression, neither platform is currently effective for delivering long-term expression of exogenous genes to B cells on a therapeutic scale.

An alternative method for introducing stable protein expression is genome editing via homology-directed repair (HDR). As described herein, in several alternatives, genome editing may be performed by HDR. Following cleavage by an engineered site-specific nuclease, DNA double-strand breaks are resolved through non-homologous end joining (NHEJ), an error-prone DNA repair pathway that typically leads to variable insertions or deletions (indels), or HDR, which repairs DNA by copying a homologous donor template. Delivery of exogenous DNA flanked by DNA homologous to the genomic sequence around the break site can lead to incorporation of the exogenous sequence in a site-specific manner. HDR-mediated genome editing in B cells may have several advantages over viral vector transduction for therapeutic applications, including decreased risk of insertional mutagenesis and sustained transgene expression. As described herein, high-efficiency HDR delivery of therapeutic transgenes to hematopoietic cells including primary human T cells and hematopoietic stem cells may be achieved, which was also a technique that is performed by others, but similar approaches are yet to be applied in modification of primary human B cells.

The clustered regularly interspaced short palindromic repeats (CRISPR)/CRISPR-associated protein 9 (Cas9) system is an RNA-guided nuclease platform that is easily engineered to efficiently target specific sites in the genome for cleavage, generating double-strand DNA breaks. The use of site-specific nucleases for gene disruption or HDR in B cells is currently limited to transformed or lymphoma-derived cell lines and murine models and has required plasmid- or LV-based CRISPR/Cas9 delivery. Here, as described in the alternatives herein, is high-efficiency genome editing in human peripheral blood B cells (75-90% gene disruption or 10-40% HDR) by delivering CRISPR/Cas9 ribonucleoprotein (RNP) complexes alone or in combination with single-stranded DNA oligonucleotide (ssODN) or adeno-associated virus (AAV) repair templates, respectively. As shown in the alternatives herein, edited primary B cells can subsequently be differentiated in culture into plasma cells that produce physiological doses of therapeutic proteins including human factor IX (FIX).

### Conditions for Expansion of Primary Naïve Human B Cells

Rapid cell cycling and/or persistence in the S/G2 phases of the cell cycle promote HDR in both cell lines and primary hematopoietic cells. Based on previous reports demonstrating rapid expansion of primary human B cells ex vivo, a combination of stimulants were initially used (hereafter called “B cell activation cocktail”) that included artificially oligomerized CD40 ligand (MEGACD40L®; two linked CD40L trimers) in association with CpG, IL-2, IL-10, and IL-15. Primary human CD19+ B cells from peripheral blood mononuclear cells (PBMCs) were isolated and cultured them for 13 days with this B cell activation cocktail. This treatment resulted in a ˜36-fold expansion of B cells (FIG. 40), while preserving viability at >60% (FIG. 41). Most cells maintained a naïve B cell phenotype (CD27⁻CD138⁻CD38^{low/-}CD19^{high}IgM⁺IgD⁺; FIG. 40C) over the 13-day culture; however, IgD expression was gradually downregulated (FIG. 40C), demonstrating some ex vivo skewing due to stimulation. Together, these data demonstrate that the B cell activation cocktail facilitates rapid cycling of naïve and activated primary human B cells.

### Cas9-Mediated Disruption of CCR5 and PRDM1 in Primary Human B Cells

To assess the efficiency of Cas9-induced indels in B cells, CRISPR guide RNAs (gRNAs) were designed that target CCR5 (which is not expressed in human B cells and has no known significance in plasma cell development) or PRDM1 (encoding BLIMP1, a protein required for B cell differentiation into plasma cells). After optimizing electroporation using mRNA (FIG. 41), 30 pmol Cas9-guide ribonucleoprotein (RNP) complexes were transfected into B cells, cultured them for five additional days under activating conditions and extracted total genomic DNA to assess nuclease-induced indels using the T7 endonuclease 1 assay (FIG. 42A). Sequencing confirmed on-target indels and revealed that the RNPs induced high indel frequencies at each guide target site (43-84%; FIG. 42A). Western blot also verified concomitant PRDM1 protein reduction in the PRDM1-expressing TMD8 lymphoma cell line (FIG. 47A; ˜65% knockdown). Finally, it was discovered that B cells remained viable despite these genome modifications (FIG. 47B). These data show that high-efficiency Cas9-mediated gene disruption is achievable in primary human B cells.

HDR-Mediated Single-Nucleotide Substitution at the PRDM1 Locus Using an ssODN Donor Template

Cas9-induced DNA lesions can be seamlessly repaired via the HDR pathway in the presence of a donor template with homology sequences flanking the lesion. An ssODN was initially tested as a donor template based on promising results in other cell types. A 120-base ssODN containing asymmetric homology arms (89 bases 5′ and 30 bases 3′) was designed that flank the PRDM1g-2 target site, with a single-nucleotide change at the 90^{th} position that mutates the last nucleotide of the protospacer adjacent motif (PAM; GGG to GGT; FIG. 42B). This single nucleotide change was designed to both prevent Cas9-mediated cleavage of the repaired sequence and to serve as a molecular marker for HDR. In this experiment, primary human B cells were activated for two days and then transfected with Cas9 RNP in conjunction with various doses of the ssODN. Two and five days following transfection, cells transfected with <30 pmol ssODN had comparable viabilities to Cas9 RNP transfected control cells (FIG. 42C). In cells receiving 15 or 30 pmol ssODN, sequencing of the PRDM1 target region on day 5 post-transfection revealed 20-22% of alleles had undergone HDR while another 37-41% had indels (FIG. 42D), an overall editing rate marginally higher than that observed in the Cas9 RNP control (FIG. 42D). Thus, ssODN donor templates can be used to achieve high rates of HDR in primary human B cells with low cytotoxicity.

Cas9-Mediated Disruption of Genes that Regulate Plasma Cell Development

It was then investigated whether Cas9-induced gene disruption can be used to study gene roles in human plasma cell development and antibody production. An experimental workflow was developed that includes inducing NHEJ-mediated gene disruption in activated naïve human B cells using Cas9 RNPs and subsequently differentiating naïve B cells into plasma cells (CD19^{low}CD38^{high}CD27⁺ CD138⁺)using a three-step plasma cell culture system (see FIG. 44A). As a proof of concept, four genes encoding transcription factors previously reported to regulate plasma cell development in murine studies were studied: IRF4 and PRDM1/BLIMP1 are required for plasma cell differentiation (FIG. 44B), while PAX5 and BACH2 antagonize plasma cell differentiation in mice (FIG. 44B). CRISPR guides targeting each of these four genes were designed, transfected primary B cells with Cas9 RNP targeting each gene independently and subsequently induced in vitro plasma cell differentiation (FIG. 44A). As a control, cells were also transfected with Cas9 RNPs targeting CCR5. Following differentiation, significantly lower percentages of CD19^{low}CD38^{high} and CD27⁺CD138⁺ plasma cells in cultures transfected with PRDM1- or IRF4-targeting RNPs compared to cells transfected with CCR5-targeting RNP or mock transfected cells were detected (mean indel percentages=89%, 83% and 92% at the PRDM1, IRF4 and CCR5 target sites, respectively; FIG. 44C, 44D). As antibody secretion is a major function of plasma cells, IgM and IgG levels were measured in cell culture supernatants by enzyme-linked immunosorbent assay (ELISA) and found IgG to be significantly decreased in the PRDM1- and IRF4-targeted B cell cultures (FIG. 44E). Together, as predicted, these data imply that both PRDM1 and IRF4 are required for human plasma cell differentiation and antibody production. Conversely, it was observed that there was significant increases in the percentages of plasma cells in cultures transfected with eitherPAX5 orBACH2-targeting RNPs (mean indel percentages=80% and 86% at the PAX5 and BACH2 target sites, respectively; FIG. 44C, 44D). Concomitant increases in IgM and IgG secretion in PAX5-targeted cultures were also observed (FIG. 44E). Again, as predicted, these data indicate that Cas9-mediated disruption of PAX5 or BACH2 enhances human plasma cell differentiation. Further, the IgG/IgM ratio in BACH2-targeted B cell cultures was markedly lower than mock or CCR5-targeted cultures (FIG. 44E, FIG. 45A) and plasmablasts in PAX5-targeted cultures exhibited decreased surface CD19 expression (FIG. 45B), both consistent with previous studies in mice. Taken together, these results demonstrate that CRISPR/Cas9-induced gene disruption in primary B cells is useful for interrogating gene products that may modulate human plasma cell development and function.

### Site-Specific HDR at the PRDM1 Locus Using Co-Delivery of RNP and AAV Donor Template

HDR using ssODN donor templates is not suitable for delivering payloads larger than ˜400 bases due to current limitations of the fidelity of ssODN synthesis. In contrast, adeno-associated virus (AAV) can package up to ˜4.7 kilobases (kb) of ssDNA donor template. Many have used AAV to deliver candidate HDR templates leading to high levels of HDR in multiple cell types and at a variety of loci. Of note, there is no published data regarding the capacity of AAV to transduce primary human B cells. To investigate AAV transduction efficiency in human B cells, a self-complementary AAV (scAAV) with a green fluorescent protein (GFP) coding sequence driven by MND, a robust retroviral-derived ubiquitous promoter was designed. Activated B cells were transduced with this vector packaged using various serotypes and quantified GFP expression two days post transduction by flow cytometry. Regardless of serotype, it was observed that there was minimal loss of cell viability following viral exposure (FIG. 51A). It was observed that the highest percentage of GFP+ cells (mean=43%) and highest mean fluorescence intensity in B cells were transduced with AAV serotype 6 (FIG. 47A).

Because the packaging size of single-stranded AAV (ssAAV) is greater than scAAV, the efficacy of HDR-mediated integration of larger payloads by delivering ssAAV into B cells was next assessed. Initially, an AAV6 donor template containing an MND-driven GFP cassette flanked by 400 bp homology arms at the PRDM1g-2 target site (PRDM1-GFP was designed; FIG. 47B). The PAM was not included in the repair template, thereby rendering the repaired sequence non-cleavable by Cas9 (FIG. 47B). After activation and electroporation with or without Cas9 RNP, B cells were transduced with AAV6 PRDM1-GFP at various MOI and cultured for 11 additional days with the same activating factors. Although it was initially observed that a 30% viability loss at the highest AAV dose, these cultures eventually recovered and exhibited comparable viabilities by day 11 (FIG. 46B). At the highest AAV MOI, it was observed that there was persistent GFP expression in ˜10% of cells that received both the PRDM1-targeting RNP and AAV repair template, while 2% of cells that were treated with the AAV alone had persistent GFP expression (FIG. 47C). In addition, cells were co-treated with the PRDM1-targeting RNP and a non-targeting AAV BFP control (MND-BFP without homology arms; BFP-noHA) and observed ˜1% BFP expression at the endpoint (FIG. 47C), indicating that the majority of GFP expression resulting from co-delivery of PRDM1-targeting RNP and PRDM1-GFP was likely driven by HDR-mediated integration. It was also found that higher AAV doses correlated with increased percentages of GFP+ cells. Finally, it was found that increasing homology arm lengths from 400 bp to 1.0 kb did not lead to higher levels of HDR (measured as persistent GFP expression; FIG. 48A, 48B). Together, these data support the conclusion that co-delivery of Cas9 RNP and AAV can lead to efficient, targeted genomic integration of transgene, likely via the HDR pathway.

### Generation of Active FIX-Secreting Human Plasma Cells Via HDR-Mediated Integration at the CCR5 Locus

Engineering exogenous protein production in plasma cells may have therapeutic applications in protein deficiency diseases such as hemophilia B, which is caused by a deficiency of FIX. To assess whether primary B cells could be engineered to secrete active proteins with predicted functionality, HDR-mediated knock-in strategy was used to engineer de novo expression of FIX. Of note, our HDR studies targeting the PRDM1 locus (described above) were anticipated to underrepresent HDR efficiencies due to the negative impact of PRDM1 disruption on plasma B cell differentiation and the relative inefficiency of the guide used in the studies (PRDM1g-2; FIG. 42A). Therefore, the CCR5 locus as a “safe harbor” was targeted because: (a) it is not transcriptionally active in human B cells; (b) it is not required for plasma cell differentiation (FIG. 44A-D); and (c) heterozygous and homozygous null mutations in CCR5 are innocuous to human carriers. To generate plasma B cells capable of secreting exogenous human FIX, an AAV vector containing an MND-driven FIX (human codon-optimized FIX-R338L Padua variant) expression cassette was co-delivered, with the same flanking CCR5 homology arms as described above (CCR5-FIX), into primary human B cells with or without CCR5-targeting RNP (FIG. 49A). To also boost plasma cell differentiation in these cultures, PAX5-targeting RNPs were also co-delivered in a subset of CCR5-targeted cells. Although it was observed that similar HDR rates of ˜15-20% at the CCR5 locus across conditions (FIG. 49B), following plasma cell differentiation it was observed that increases in CD19^{low}CD38^{high} plasmablast percentages in the cultures that received both CCR5- and PAX5-targeting RNPs (FIG. 49C). With or without delivery of PAX5-targeting RNPs, high levels of de novo FIX secretion were detected in the cultures (FIG. 49D). In addition, dual-edited HDR cultures co-treated with both CCR5- and PAX5-targeting RNPs exhibited similar levels of FIX production (FIG. 49D).

To assess whether the FIX produced by gene-edited plasma cells is active, cultures were supplemented with vitamin K1 to enable vitamin K-dependent post-translational carboxylation of FIX and subsequently measured FIX activity levels using a chromogenic assay. It was found that FIX activity was significantly higher in the vitamin K1-supplemented, gene-edited plasma cell cultures (FIG. 49E). Despite low levels of activity in the BFP-noHA negative controls, this background was absent when FBS was completely removed from cultures, suggesting that this is likely non-specific assay background originating from murine FIX present in FBS. More importantly, FIX produced by gene-edited plasma cells exhibited high specific activity (˜63 IU/mg protein) in a vitamin K-dependent manner (FIG. 49F). These data demonstrate production of highly functional FIX via targeted HDR in gene-edited human B cells using Cas9 RNP and AAV and illustrate the possibility of multi-locus modifications to simultaneously drive plasma cell differentiation and de novo protein secretion.

### Engineered BAFF Secretion Via HDR Improves Plasma Cell Survival In Vitro

In order to use gene edited plasma cells in a therapeutic setting, their long-term survival will be required. One strategy to improve plasma cell survival is to engineer cells that secrete survival factors in an autocrine manner. As a first test of this approach, HDR-mediated knock-in strategy of the alternatives herein was used to engineer de novo expression of the B-cell activating factor (BAFF). Two AAV6 repair templates were designed: one contained MND-driven GFP and BAFF linked with a T2A self-cleaving peptide (CCR5-GFP-BAFF) and the other contained only an MND-BAFF expression cassette (CCR5-BAFF). Both donor constructs contained 800 bp homology arms around the CCR5 guide target site (FIG. 51A). Following RNP transfection and/or AAV transduction, cells were cultured using the three-step plasma cell culture system (FIG. 44A). As in earlier PRDM1-targeting experiments, it was observed that persistent GFP expression (mean=33% GFP+) only in the condition containing both CCR5-targeting RNP and AAV (FIG. 51B, FIG. 50). Regardless of the template used, it was observed that there was similar rates of HDR as quantified by digital PCR (˜28-31% HDR; FIG. 51C). While use of both donor templates led to BAFF secretion, B cells targeted by CCR5-BAFF (repair template solely containing BAFF) exhibited much higher levels of BAFF secretion (4.1 fold increase) relative to those targeted by CCR5-GFP-BAFF (template containing BAFF downstream of the T2A linkage) (FIG. 51D). To determine if this exogenous BAFF is functional, cell expansion and plasmablast formation was assessed following genome editing and in vitro differentiation. In the CCR5-BAFF edited culture that contained the highest concentration of exogenous BAFF, increases in cell number was observed, viability and percentage of CD19^{low}CD38^{high} plasmablasts relative to controls that lacked Cas9 RNP (FIG. 51E, 51F). This is consistent with previous studies showing that BAFF promotes both human and murine B cell survival and differentiation. Taken together, these results show that Cas9 RNP- and AAV-mediated genome editing can be used to generate functional, BAFF-secreting human plasma cells with improved in vitro survival.

Described herein are high rates of gene disruption in primary human B cells at multiple loci using CRISPR/Cas9 RNPs. This method is applied to modeling the impact of genetic changes on human plasma B cell differentiation. It is also demonstrated that the use of Cas9 RNP in combination with ssODN or AAV6 repair templates achieves high-efficiency HDR in B cells and to engineer secretion of functional and therapeutically relevant proteins. Importantly, gene-modified cells obtained a plasma cell phenotype following 13 days of culture (including 2 days pre-editing) in vitro and are detectable in humanized mice for an additional 3 weeks, providing evidence of the utility of this approach for adoptive cell therapies using engineered human plasma cells.

Cas9-mediated disruption of PRDM1, IRF4, PAX5, orBACH2, which either promote (PRDM1, IRF4) or inhibit (PAX5, BACH2) plasma cell differentiation in mice, led to human B cell phenotypes consistent with these regulatory roles. The ability to easily disrupt genes or induce single-base changes using Cas9 RNP with or without ssODN-mediated HDR will facilitate further study of the impact of genetic changes on human B cell development. When combined with differentiation in vitro, this system could be used as a high-throughput model for studying genetic mutations that alter human B cell development, including somatic and/or germ-line mutations associated with a range of human B cell disorders (in B cell lymphoma and in autoimmune disorders including systemic lupus erythematosus); as well as a range of mechanistic studies designed to assess candidate genes identified by genome-wide association studies.

The development of a method for the efficient, site-specific introduction of transgenes via HDR opens the possibility of engineering plasma cells to act as autologous “cell factories,” capable of delivering sustained, high doses of therapeutic proteins to patients. As a proof of concept, plasma cells were engineered to secrete FIX. Deficiency of FIX is the cause of hemophilia B, a genetic disease characterized by blood clotting defects. Current treatment for hemophilia B is limited to protein replacement therapy, which is costly and non-curative. Recent gene therapy trials reported long-term FIX expression in patients after intravenous injection of a liver-tropic AAV8-FIX vector; however, pre-existing AAV neutralizing antibodies to all known AAV serotypes are prevalent in humans and greatly limit in vivo AAV transduction, making a significant group of patients with anti-AAV antibodies ineligible for treatment. In addition, systemic AAV delivery induces humoral immunity against AAV that prevents subsequent delivery, thus limiting AAV therapy to a single dose and/or necessitating use of alternative serotypes. Delivery of ex vivo gene-edited plasma cells producing FIX would remove the requirement for systemic AAV delivery and likely avoid limitations posed by AAV neutralizing antibodies.

In addition, immune responses to therapeutic proteins comprise a key unaddressed challenge. Based on evidence that B cell delivery can induce tolerance, an additional application of B cell editing technology would be to deliver smaller numbers of short-lived B cells producing FIX, or other immunogenic proteins, with the goal of inducing tolerance to the secreted product.

The engraftment studies contrast with the majority of those previously reported using human cells. In most other successful studies, human stem cells have been engrafted into neonatal humanized murine models, including co-engraftment with human liver and thymus (pelanta review). In these cases, CD34⁺ stem cells can differentiate into B cells and even mature B cells. However, the majority of the engrafted cells remain in the transitional and/or immature developmental stages. Further, the demonstration that engineering mature B cells to express human BAFF confers a significant engraftment advantage parallels studies showing that access to human cytokines, including IL6, SIRPα, or T cell factors promote B cell maturation and the ability to respond to immunization in mice.

In summary, the alternatives herein, demonstrate high rates of locus-specific genome editing in primary human B cells. These engineered B cells retain the ability to differentiate into plasma cells ex vivo and secrete physiological doses of therapeutic proteins such as FIX. The ability to efficiently target specific loci enables us to drive B cell differentiation and secretory programs. Thus, creative multiplexing of gene disruption and HDR will provide ever more powerful tools to coordinately optimize protein secretion, cellular phenotype and long-term survival.

CRISPR/Cas9 Reagents and ssODNs

crRNAs targeting the CCR5, PRDM1, IRF4, PAX5, BACH2 loci were identified using the MIT CRISPR design tool and the Broad Institute sgRNA design tool and synthesized (IDT®) containing phosphorothioate linkages and 2′O-methyl modifications. Single-stranded oligonucleotides (ssODNs) were commercially synthesized by IDT® (Ultramer® DNA Oligonucleotides) with phosphorothioate linkages. crRNA and tracrRNA (IDT®) hybrids were mixed with Cas9 nuclease (IDT®) at a 1.2:1 ratio and delivered with or without ssODNs to cells by Neon electroporation (ThermoFisher Scientific®).

### Production of Recombinant AAV Vectors

The AAV vector, serotype helper and HgT1-adeno helper plasmids were transfected into HEK293T cells. Cells were harvested 48 hours later, lysed by 3 freeze-thaw cycles, and the cell lysate was treated with benzonase. Virions with recombinant AAV genomes were purified using an iodixanol density gradient. All multiplicity of infection (MOI) calculations were based on qPCR-based titers of AAV genomes using ITR specific primers and probe.

### Samples and Primary Human B Cell Genome Editing

Peripheral blood mononuclear cells (PBMCs) were collected from whole blood of consented donors and cryopreserved. CD19+ B cells were subsequently isolated by negative selection (Pan-B cell kit, Miltenyi Biotec®) and cultured in Iscove's modified Dulbecco's medium (IMDM; Thermo Fisher Scientific®) supplemented with 10% fetal bovine serum (FBS) and 55 μM beta-mercaptoethanol at 1-1.5×10⁶ cells/ml. B cells were activated with 100 ng/ml of recombinant human MEGACD40L® (Enzo Life Sciences®), 1 pg/ml of CpG oligodeoxynucleotide 2006 (Invitrogen®), 50 ng/ml of IL2 (Peprotech®), 50 ng/ml of IL-10 (Peprotech®) and 10 ng/ml of IL15 (Peprotech®) for two days. Cells were then electroporated with Cas9 RNP complexes. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days.

### Flow Cytometry

Flow cytometric analysis was done on an LSR II flow cytometer (BD Biosciences) and data were analyzed using FlowJo software (TreeStar). Flow cytometry gating for fluorescent proteins (FIG. 52A), viability (FIG. 52B) and immunophenotyping (FIG. 52C) are described.

### Plasma Cell Differentiation Culture

Plasma cells were differentiated in vitro using a three-step culture system as previously described. CD19+ B cells were activated and gene-edited as outlined above and expanded for five days in the same cocktail. Following washing with 1×PBS, the cells were seeded in medium with IL-2 (50 ng/ml), IL-6 (50 ng/ml), IL-10 (50 ng/ml) and IL-15 (10 ng/ml) for three days. Next, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the cells were seeded in medium with IL-2 (50 ng/ml), IL-6 (50 ng/ml), IL-10 (50 ng/ml) and IL-15 (10 ng/ml) for three days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation

### ELISA

3 days prior to measuring culture protein levels, cells were collected, washed with PBS and then resuspended in new culture medium at 1×10⁶ cells/ml. After 3 days, the culture supernatant was collected and protein secretion levels were determined by enzyme-linked immunosorbent assay (ELISA) for IgG and IgM (Ready-SET-GO®, eBioscience®, San Diego, CA), BAFF (BAFF/BLyS/Quantikine ELISA Kit, R&D Systems®, Minneapolis, MN) and FIX (FIX Human ELISA Kit, Abcam®, Cambridge, MA).

### FIX Chromogenic Assay

Activity of recombinant FIX expressed by gene-edited B cells was assessed using a chromogenic assay (Rox Factor IX, 900020). The procedure was performed following the manufacturer's instructions. Absorbance values were measured using a VICTOR3TM plate reader (PerkinElmer®). A human normal pooled plasma standard was used to establish the calibration curve.

To assess FIX activity in edited plasma cell cultures, B cells were edited and differentiated in vitro. At day 8 post genome editing, FBS content was reduced from 10% to 2% of culture to reduce assay background. Insulin, transferrin, sodium selenite were added to cultures to maintain cell survival in the reduced FBS environment. Finally, in specified cultures that were supplemented with vitamin K1 5 μg/ml of vitamin K1 was added to the medium for generating active FIX via vitamin K-dependent carboxylation. 0.005% was used to increase solubility of vitamin K1.

### NSG Mouse Transplant

NOD/SCID/gamma-c null (NSG) mice were purchased from Jackson Laboratories and maintained in a designated pathogen-free facility at the Seattle Children's Research Institute (SCRI). All animal studies were performed according to the Association for Assessment and Accreditation of Laboratory Animal Care (AAALAC) standards, and were approved by the SCRI Institutional Animal Care and Use Committee (IACUC).

In the NSG transplant experiment, NSG mice were conditioned with 25 mg/kg of Busulfan (Selleckchem®) via intraperitoneal injection. 24 hours after conditioning, 10×10⁶ in vitro differentiated plasma B cells, either mock or edited BAFF-expressing, were delivered into each 6- to 8-week-old NSG recipient via retro-orbital infusion. Mice were bled at day 10 and sacrificed at day 21 post infusion. All peripheral blood samples were collected in serum separator tubes for serum collection.

### Statistical Analysis and Data Availability

Statistical analyses were performed using Graphpad Prism® 7 (GraphPad®, San Diego, CA). Because there wasn't obvious skewing in any of the conditions and there was minimal variation within conditions, it is assumed all data followed a normal distribution. p values in multiple comparisons were calculated using one-way ANOVA with the Sidak correction; p values in comparisons between two groups were calculated using paired two-tailed t-test. Values from independent experiments are shown as means±SEM. All sequencing data are accessible at the NCBI Sequence Read Archive through the following accession number: SRP113557.

Methods for Making Plasma Cells or Plasma Cell Precursors that Express a Macromolecule, Such as a Protein, Protein Mimetic or a Peptide, and Making a Long Lived Plasma Cell.

Described herein are methods for making long lived plasma cells. In some alternatives, these plasma cells can also express a desired macromolecule, such as a protein, an antibody, enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. These cells can then be used for treatment, amelioration, or inhibition of a disease such as cancer by e.g., utilizing immunotherapy.

The method of making a plasma cell or plasma cell precursor that expresses a molecule, such as a macromolecule is provided in several alternatives herein. The method comprises isolating B cells, developing the B cells, performing a first round of genome editing of the B cells for protein expression in absence of viral integration, expanding the B cells; and differentiating the B cells, optionally, after step (c) or (d), thereby producing plasma cells or plasma cell precursors that express the molecule. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells in step (a) comprise memory B cells and/or naïve B cells. In some alternatives, the developing of the B cells is performed after the B cells are arrested at a specific phase of development and/or activating the B cells such that the B cells are permissive for recombination without further B cell differentiation. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cell. In some alternatives, the method to prevent somatic hypermutation of an antibody locus in the B cell comprises disruption of an AID gene. In some alternatives, the method further comprises selectively increasing a proportion of gene edited B cells. In some alternatives, the step of increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment, amelioration, or inhibition of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding fragment thereof is specific for a protein or other molecule expressed in autoimmune disorders, autoinflammatory disorders, immune dysregulation and cancer. In some alternatives, the antibody or binding fragment thereof is an anti-IL-1 monoclonal antibody, anti-TNF antibody, anti-IL-33 antibody or anti-C5 antibody or a binding fragment of one or more of these antibodies. In some alternatives, the protein is an anti-thrombotic molecule, wherein the anti-thrombotic molecule is APLN. In some alternatives, the protein comprises an anti-PCSK9 inhibitory antibody or a binding fragment thereof. In some alternatives, the protein comprises broadly neutralizing HIV-1 antibodies (bNAbs) or a binding portion thereof. In some alternatives, the protein comprises a prodrug or a proenzyme. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or binding portion thereof is specific for an antigen that is expressed in a virus, fungus, parasite or bacteria. In some alternatives, the protein comprises an antibody or portion thereof, wherein the antibody or portion thereof is specific for an antigen that is express in a viral, fungal, parasitic or bacterial infection. In some alternatives herein, the genome editing is performed by nonpathogenic AAV mediated editing by direct homolougous recombination. In some alternatives herein, the protein comprises an enzyme, monoclonal antibody or a binding portion thereof, neutralizing antibodies or a binding portions thereof, therapeutic antibodies or binding portions thereof, cytokine, cytokine receptor, complement protein, inhibitory protein, anti-fibrotic molecule, anti-thrombotic molecule, coagulation factor, glucose response element or a synthetic engineered protein. In some alternatives herein, the protein is Factor VIII, Factor IX, SERPING1 or SERPINA1. In some alternatives herein, the enzyme is ADAMTS13, LIPA, GLA, or ALPL. In some alternatives herein, the protein is a receptor antagonist for treatment of periodic fever/autoinflammatory syndromes or complement inhibitory proteins. In some alternatives herein, the compliment inhibitory protein is Factor H, Factor I or a C1 inhibitor. In some alternatives herein, the protein is an anti-fibrotic molecule, wherein the anti-fibrotic molecule is SCGB1A1. In some alternatives herein, the therapeutic antibody or a binding portion thereof is specific for a protein or other molecule expressed in autoimmune disorders, autoinflammatory disorders, immune dysregulation and/or cancer. In some alternatives herein, the antibody is an anti-IL-1 monoclonal antibody, anti-TNF antibody, anti-IL-33 antibody or anti-C5 antibody or a binding portion of any one or more of these antibodies. In some alternatives herein, the protein is an anti-thrombotic molecule, wherein the anti-thrombotic molecule is APLN. In some alternatives herein, the protein comprises an anti-PCSK9 inhibitory antibody or a binding portion thereof. In some alternatives herein, the protein comprises broadly neutralizing HIV-1 antibodies (bNAbs), or a binding portion thereof. In some alternatives herein, the protein is an engineered protein, wherein the engineered protein is protective in viral, fungal, parasitic or bacterial infection. In some alternatives herein, the engineered protein comprises an antibody or a binding portion thereof, wherein the antibody or a binding portion thereof is specific for an antigen that is expressed by a virus, fungus, parasite or bacteria. In some alternatives herein, the protein comprises an antibody or a binding portion thereof, wherein the antibody or a binding portion thereof is specific for an antigen that is expressed on a viral, fungal, parasitic or bacterial infection. In some alternatives herein, the method further comprises purifying the plasma cells or plasma cell precursors after step e) by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection. Novel aspects of the alternatives described herein can include, but are not limited to: (1) the use of blood-derived human B cells as a starting material for a plasma cell protein producing immunotherapy; (2) RNA- and protein-based transfection to facilitate delivery of candidate designer nucleases targeting a broad range of genetic loci in primary B cells that include, but are not limited to zinc finger nucleases, transcription activator-like effector nucleases (TALEN), homing endonucleases (HEs), combined TALEN-HE proteins (megaTALs) and clustered regularly interspersed short palindromic repeat DNA (CRISPR) coupled to the CAS nuclease; (3) transfection of long single-stranded DNA oligonucleotides or transduction with recombinant adeno-associated virus to facilitate efficient delivery of donor DNA templates carrying therapeutic expression cassettes into primary human B cells in order to facilitate efficient homologous recombination into a range of candidate genetic loci; (4) integrity measures that include, but are not limited to, methods to prevent somatic hypermutation of the B cell antibody locus during the engineering process including, but not limited, to disruption of the AID gene; (5) production enhancers that include, but are not limited to methods to introduce dimerizable drug-inducible activating proteins to enable selectable expansion of engineered human B cells in vitro or in vivo; (6) safety measures that include, but are not limited to, introduction of sequences from cell surface proteins including, but not limited to, the CD20 protein into B cells to enable targeted removal from recipients using rituxan® or alternative therapeutic approaches; and (7) the use of a proprietary multi-step cytokine and co-culture based systems to facilitate differentiation of blood-derived B cells into long-lived plasma cells and their survival and expansion in vitro.

In some alternatives, the plasma cell or plasma cell precursor expresses a macromolecule, such as a protein, wherein the protein is used in a protocol such as immunotherapy, as envisioned below:
(1) Prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites.
(2) Protein replacement, enzyme replacement and rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia).
(3) Immune modulation via expressed cytokines, cytokine receptors, complement proteins or other inhibitory proteins including, but not limited to: Ill receptor antagonist for the treatment or inhibition of periodic fever/autoinflammatory syndromes; complement inhibitory proteins (including Factor H, Factor I) for the treatment or inhibition of atypical hemolytic uremic syndrome/membranoproliferative glomerulonephritis; and/or C1 inhibitor for hereditary angioedema.

Anti-fibrotic molecules including, but not limited to SCGB1A1 for the treatment or inhibition of pulmonary fibrosis.

Therapeutic antibodies or a binding portion thereof for autoimmune disorders, autoinflammatory disorders, immune dysregulation and/or cancer including but not limited to: anti-IL-1 monoclonal antibodies or a binding portion thereof for treatment or inhibition of periodic fever/autoinflammatory syndromes; anti-TNF antibodies or a binding portion thereof for inflammatory arthritis/inflammatory bowel disease, anti-IL-33 antibodies or a binding portion thereof for the treatment or inhibition of asthma and anti-C5 antibodies or a binding portion thereof for treatment or inhibition of paroxysmal nocturnal hemoglobinuria/atypical HUS.

Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein).

(7) Glucose responses elements upstream of insulin for treatment or inhibition of diabetic conditions.

(8) Therapeutic monoclonal antibodies or a binding portion thereof for the treatment or inhibition of hyper-cholesterolemia, including anti-PCSK9 inhibitory antibodies or a binding portion thereof.

### Methods for Making a Long Lived Plasma Cell.

Methods for making a long lived plasma cell are also provided. Long lived plasma cells that express protein can survive in a subject in a need for a treatment or inhibition of a disease such as cancer and such approaches eliminate the need to provide administering of cells within a short time frame.

The method of making a long lived plasma cell, comprises: isolating B cells; activating the B cells; a first round of genome editing of the B cells for molecule expression in the absence of viral integration; expanding the B cells; and differentiating the B cells. In some alternatives, the B cells in the isolating step comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, first round of genome editing comprises transducing the B cell with a recombinant adeno-associated virus vector for homologous recombination into a candidate genetic loci. In some alternatives, method further comprises applying methods to prevent somatic hypermutation of an antibody locus and other target loci in the B cell. In some alternatives, the method to prevent somatic hypermutation of an antibody locus in the B cell comprises disruption of an AID gene. In some alternatives, the method further comprises introducing production enhancers into the B cell or disruption of candidate loci within the B cell to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment, amelioration, or inhibition of periodic fever/autoinflammatory syndromes, complement inhibitory proteins (including Factor H, Factor I) for treatment, amelioration, or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody or binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives, the plasma cell or plasma cell precursor that expresses a macromolecule, such as a protein, protein mimetic or peptide is used in a therapeutic protocol such as immunotherapy as envisioned below:

(1) Prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites.

(2) Protein replacement, enzyme replacement and rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia).

(3) Immune modulation via expressed cytokines, cytokine receptors, complement proteins or other inhibitory proteins including, but not limited to: Ill receptor antagonist for the treatment or inhibition of periodic fever/autoinflammatory syndromes; complement inhibitory proteins (including Factor H, Factor I) for the treatment or inhibition of atypical hemolytic uremic syndrome/membranoproliferative glomerulonephritis; and/or C1 inhibitor for hereditary angioedema.

(4) Anti-fibrotic molecules including, but not limited to SCGB1A1 for the treatment or inhibition of pulmonary fibrosis.

(5) Therapeutic antibodies or a binding portion thereof for autoimmune disorders, autoinflammatory disorders, immune dysregulation and/or cancer including but not limited to: anti-IL1 monoclonal antibodies or a binding portion thereof for treatment or inhibition of periodic fever or autoinflammatory syndromes; anti-TNF antibodies or a binding portion thereof for inflammatory arthritis/inflammatory bowel disease, anti-IL-33 antibodies or a binding portion thereof for the treatment or inhibition of asthma and/or anti-C5 antibodies or a binding portion thereof for the treatment or inhibition of paroxysmal nocturnal hemoglobinuria/atypical HUS.

(6) Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein).

(7) Glucose responses elements upstream of insulin for the treatment or inhibition of diabetic conditions.

(8) Therapeutic monoclonal antibodies or a binding portion thereof for the treatment or inhibition of hyper-cholesterolemia, including anti-PCSK9 inhibitory antibodies or a binding portion thereof.

Plasma Cells that Express Protein

Plasma cells plasma cell precursors that express macromolecules such as protein, protein mimetics or peptides are provided by the alternatives herein. Plasma cells are provided wherein the plasma cells are also manufactured by the alternative methods described herein. The plasma cell that expresses the macromolecule is long-lived and can be expected to survive within either the bone marrow or within the spleen. In some alternatives, the macromolecule is a protein, protein mimetic or a peptide. The plasma cells created as described herein can persist in non-dividing state within bone marrow survival niche without need for ongoing antigen exposure. They are also relatively resistant to immunosuppression/chemotherapy. Additionally, the plasma cells can be used in conjunction with CAR T cell therapy, which can be used in subjects in need that are suffering from a disease such as cancer, a bacterial or a viral infection so as to treat, ameliorate, or inhibit the disease, cancer or bacterial or viral infection. In some alternatives, the plasma cell expresses CD20.

In some alternatives, a plasma cell that expresses a molecule such as a macromolecule, is provided. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the cell is derived from a B cell. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or monoclonal antibody. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for the treatment or inhibition of periodic fever/autoinflammatory syndromes, complement inhibitory proteins (including Factor H, Factor I) for treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody or binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell expresses CD20 for removal from a subject.

The plasma cell or plasma cell precursor can be manufactured by any one of the alternative methods provided herein. The method of making a plasma cell or plasma cell precursor that expresses a molecule, such as a macromolecule is provided in several alternatives herein. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. The method comprises isolating B cells, developing the B cells, performing a first round of genome editing of the B cells for protein expression in absence of viral integration, expanding the B cells; and differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the B cell in the isolating step comprises B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the guide sequence comprises a sequence set forth in any one of SEQ ID NO: 2-13, 20-25 or 63-112. In some alternatives, the Cas nuclease comprises Cas1, Cas2, Cas3, Cas4, Cas5, Cas6, Cas7, Cas8 or Cas9. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cell. In some alternatives, the method to prevent somatic hypermutation of an antibody locus in the B cell comprises disruption of an AID gene. In some alternatives, the method further comprises selectively increasing a proportion of gene edited B cells. In some alternatives, the step of increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed in a virus, fungus, parasite or bacteria. In some alternatives, the protein comprises an antibody or a binding portion thereof, wherein the antibody or a binding portion thereof is specific for an antigen that is expressed in a viral, fungal, parasitic or bacterial infection. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives, the plasma cell or plasma cell precursor that expresses a macromolecule is a long lived plasma cell. The macromolecule can be a protein, protein mimetic or a peptide. The plasma cell or plasma cell precursor can be manufactured by any one of the alternative methods for making a long lived plasma cell. The method of making a long lived plasma cell, comprises: isolating B cells; activating the B cells; a first round of genome editing of the B cells for protein expression in the absence of viral integration; expanding the B cells; and differentiating the B cells. In some alternatives, the B cells in the isolating step comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, first round of genome editing comprises transducing the B cell with a recombinant adeno-associated virus vector for homologous recombination into a candidate genetic loci. In some alternatives, method further comprises applying methods to prevent somatic hypermutation of an antibody locus and other target loci in the B cell. In some alternatives, the method to prevent somatic hypermutation of an antibody locus in the B cell comprises disruption of an AID gene. In some alternatives, the method further comprises introducing production enhancers into the B cell or disruption of candidate loci within the B cell to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were seeded in medium with IL-2 (50 ng/ml), IL-6 (50 ng/ml), IL-10 (50 ng/ml) and IL-15 (10 ng/ml) for three days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiationIn some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL, and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for the treatment or inhibition of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives herein, the genome editing is performed by nonpathogenic AAV mediated editing by direct homolougous recombination. In some alternatives, the plasma cell expresses CD20. In some alternatives, the plasma cell is purified by positive selection against CD138.

In some alternatives of the plasma cell or plasma cell precursor, the plasma cell or plasma cell precursor is derived from a B cell or B cell precursor. In some alternatives the plasma cell or plasma cell precursor expresses a macromolecule such as a protein, protein mimetic or a peptide. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or monoclonal antibody. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever or autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell expressing the protein is a long lived plasma cell. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives, the plasma cell or plasma cell precursor that expresses a molecule, such as protein mimetic, protein or peptide is used in a therapeutic protocol such as an immunotherapy as envisioned below:

(1) Prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites.

(2) Protein replacement, enzyme replacement and rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia).

(3) Immune modulation via expressed cytokines, cytokine receptors, complement proteins or other inhibitory proteins including, but not limited to: Ill receptor antagonist for treatment or inhibition of periodic fever/autoinflammatory syndromes; complement inhibitory proteins (including Factor H, Factor I) for treatment or inhibition of atypical hemolytic uremic syndrome/membranoproliferative glomerulonephritis; and/or C1 inhibitor for hereditary angioedema.

(4) Anti-fibrotic molecules including, but not limited to SCGB1A1 for the treatment or inhibition of pulmonary fibrosis.

(5) Therapeutic antibodies or a binding portion thereof for autoimmune disorders, autoinflammatory disorders, immune dysregulation and/or cancer including but not limited to: anti-IL-1 monoclonal antibodies or a binding portion thereof for treatment or inhibition of periodic fever/autoinflammatory syndromes; anti-TNF antibodies or a binding portion thereof for inflammatory arthritis/inflammatory bowel disease, anti-IL-33 antibodies or a binding portion thereof for the treatment or inhibition of asthma and/or anti-C5 antibodies or a binding portion thereof for the treatment or inhibition of paroxysmal nocturnal hemoglobinuria/atypical HUS.

(6) Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein).

(7) Glucose responses elements upstream of insulin for the treatment or inhibition of diabetic conditions.

(8) Therapeutic monoclonal antibodies or a binding portion thereof for the treatment or inhibition of hyper-cholesterolemia, including anti-PCSK9 inhibitory antibodies or a binding portion thereof.

### Compositions

Compositions are provided herein, wherein the composition comprises the plasma cell manufactured by any one of the alternative methods provided herein or the cell of any one of the alternative cells provided herein. The composition can comprise a plasma cell or plasma cell precursor that expresses a molecule, such as a macromolecule or a plasma cell that is also a long lived plasma cell. In some alternatives, the composition comprises the plasma cell manufactured by any one of the alternatives herein and a second B cell, wherein the second B cell secretes a macromolecule for inducing tolerance of a peptide for immunotherapeutic application or for inducing engraftment of the plasma cell manufactured by any one of anyone of the alterantives herein. In some alternatives, the macromolecule expressed by the second B cell comprises IFN-alpha, BAFF, APRIL, IL-10 or IL-6. In some alternatives, the composition comprises the plasma cell manufactured by any one of anyone of the alterantives herein or comprising the cell of anyone of the alterantives herein.

The plasma cell or plasma cell precursor can be manufactured by any one of the alternative methods provided herein. The method of making a plasma cell or plasma cell precursor that expresses a molecule, such as a macromolecule is provided in several alternatives herein. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. The method comprises isolating B cells, developing the B cells, performing a first round of genome editing of the B cells for protein expression in absence of viral integration, expanding the B cells; and differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the guide sequence comprises a sequence set forth in any one of SEQ ID NO: 2-13, 20-25 or 63-112. In some alternatives, the Cas nuclease comprises Cas1, Cas2, Cas3, Cas4, Cas5, Cas6, Cas7, Cas8 or Cas9. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the step of increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were seeded in medium with IL-2 (50 ng/ml), IL-6 (50 ng/ml), IL-10 (50 ng/ml) and IL-15 (10 ng/ml) for three days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for the treatment or inhibition of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody or a binding portion thereof is an anti-I monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed on a virus, fungus, parasite or bacteria. In some alternatives, the protein comprises an antibody or a binding portion thereof, wherein the antibody or portion thereof is specific for an antigen that is express by a viral, fungal, parasitic or bacterial infection. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives, the plasma cell or plasma cell precursor that expresses a macromolecule is a long lived plasma cell. The macromolecule can comprise protein, a protein mimetic or a peptide. The plasma cell or plasma cell precursor can be manufactured by any one of the alternative methods for making a long lived plasma cell. The method of making a long lived plasma cell, comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, first round of genome editing comprises transducing the B cell with a recombinant adeno-associated virus vector for homologous recombination into a candidate genetic loci. In some alternatives, method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were seeded in medium with IL-2 (50 ng/ml), IL-6 (50 ng/ml), IL-10 (50 ng/ml) and IL-15 (10 ng/ml) for three days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiationIn some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL, and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, IL-6, Factor VIII, Factor IX, SERPING1 or SERPINAL. In some alternatives, the enzyme is ADAMTS13, LIPA, GLA, or ALPL. In some alternatives, the protein is a receptor antagonist for the treatment or inhibition of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives, the plasma cell or plasma cell precursor that expresses a macromolecule, such as a protein, protein mimetic or peptide, in the composition is used in a therapeutic application such as immunotherapy as envisioned below:

(1) Prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites.

(2) Protein replacement, enzyme replacement and rescue of enzyme deficiencies including, but not limited to Factor VII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia).

(3) Immune modulation via expressed cytokines, cytokine receptors, complement proteins or other inhibitory proteins including, but not limited to: Ill receptor antagonist for the treatment or inhibition of periodic fever/autoinflammatory syndromes; complement inhibitory proteins (including Factor H, Factor I) for the treatment or inhibition of atypical hemolytic uremic syndrome/membranoproliferative glomerulonephritis; and/or C1 inhibitor for hereditary angioedema.

(4) Anti-fibrotic molecules including, but not limited to SCGB1A1 for the treatment or inhibition of pulmonary fibrosis.

(5) Therapeutic antibodies or a binding portion thereof for the treatment or inhibition of autoimmune disorders, autoinflammatory disorders, immune dysregulation and/or cancer including but not limited to: anti-IL-1 monoclonal antibodies or a binding portion thereof for treatment of periodic fever/autoinflammatory syndromes; anti-TNF antibodies or a binding portion thereof for inflammatory arthritis/inflammatory bowel disease, anti-IL-33 antibodies or a binding portion thereof for the treatment or inhibition of asthma and/or anti-C5 antibodies or a binding portion thereof for the treatment or inhibition of paroxysmal nocturnal hemoglobinuria/atypical HUS.

(6) Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein).

(7) Glucose responses elements upstream of insulin for the treatment or inhibition of diabetic conditions.

(8) Therapeutic monoclonal antibodies or a binding portion thereof for the treatment or inhibition of hyper-cholesterolemia, including anti-PCSK9 inhibitory antibodies or a binding portion thereof.

In some alternatives of the composition, the composition comprises a cell. In some alternatives of the plasma cell, the plasma cell is derived from a B cell. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the cell expresses a macromolecule such as a protein, protein mimetic or a peptide. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell expressing the protein is a long lived plasma cell. In some alternatives herein, the genome editing is performed by nonpathogenic AAV mediated editing by direct homolougous recombination.

Expression of a macromolecule, such as a protein, protein mimetic or peptide in a subject in need.

Described herein, are therapeutic methods for providing a desired macromolecule to a subject in need. In some alternatives, the macromolecule is a protein, protein mimetic or a peptide. The subject in need can be suffering from an enzyme deficiency, cancer, bacterial disease, viral disease or a parasitic infection. In some alternatives, the subject in need suffers from cancer or a subject having cancer is selected or identified to receive an anti-cancer therapy. In some alternatives, the cancer is a solid tumor. In some alternatives, the solid tumor is selected from the group consisting of a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer. In some alternatives, the method further comprises administering a cellular therapy to the subject in need thereof before, after or simultaneous to introducing, providing, or administering any one or more of the cells of any of the alternatives described herein. In some alternatives, the cellular therapy is CAR T-cell therapy.

The method of expressing a molecule, such as a macromolecule in a subject in need can comprise: administering a plasma cell manufactured by any one of the alternative methods herein, the plasma cell that expresses the macromolecule of any one of the alternative methods herein, or the composition of any one of the alternative methods herein into the subject in need. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity.

The methods for manufacturing the cell include the alternative methods for making a long lived plasma cell, and alternative methods for making a plasma cell or plasma cell precursor that expresses a molecule, such as a macromolecule. Compositions comprising the plasma cell of the alternatives herein can also be used.

The composition comprises the plasma cell manufactured by any one of the alternative methods provided herein or the cell of any one of the alternative cells provided herein. The composition can comprise a plasma cell or plasma cell precursor that expresses a molecule, such as a macromolecule or a plasma cell that is also a long lived plasma cell.

The plasma cell can be manufactured by any one of the alternative methods provided herein. The method of making a plasma cell or plasma cell precursor that expresses a molecule, such as a macromolecule is provided in several alternatives herein. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. The method comprises isolating B cells, developing the B cells, performing a first round of genome editing of the B cells for protein expression in absence of viral integration, expanding the B cells; and differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cells. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the guide sequence comprises a sequence set forth in any one of SEQ ID NO: 2-13, 20-25 or 63-112. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the step of increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were seeded in medium with IL-2 (50 ng/ml), IL-6 (50 ng/ml), IL-10 (50 ng/ml) and IL-15 (10 ng/ml) for three days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for the treatment or inhibition of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL-1 monoclonal antibody, anti-TNF antibody, anti-IL-33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed in a virus, fungus, parasite or bacteria. In some alternatives, the protein comprises an antibody or a binding portion thereof, wherein the antibody or a binding portion thereof is specific for an antigen that is expressed in a viral, fungal, parasitic or bacterial infection. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives, the plasma cell or plasma cell precursor that expresses a macromolecule is a long lived plasma cell. In some alternatives, the macromolecule is a protein, protein mimetic or a peptide. The plasma cell can be manufactured by any one of the alternative methods for making a long lived plasma cell. The method of making a long lived plasma cell, comprises: isolating B cells; activating the B cells; a first round of genome editing of the B cells for protein expression in the absence of viral integration; expanding the B cells; and differentiating the B cells. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cells. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, first round of genome editing comprises transducing the B cell with a recombinant adeno-associated virus vector for homologous recombination into a candidate genetic loci. In some alternatives, method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were seeded in medium with IL-2 (50 ng/ml), IL-6 (50 ng/ml), IL-10 (50 ng/ml) and IL-15 (10 ng/ml) for three days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL, and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives of the composition, the composition comprises a cell. In some alternatives of the plasma cell, the plasma cell is derived from a B cell. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the cell expresses a macromolecule such as a protein, protein mimetic or a peptide. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or monoclonal antibody. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell expressing the protein is a long lived plasma cell. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives of the method of expressing a macromolecule in a subject in need, the administering is performed by adoptive transfer. In some alternatives, the macromolecule is a protein, protein mimetic or a peptide. In some alternatives of the method of expressing protein in a subject in need, the subject has received a stem cell administration or solid organ transplantation. In some alternatives of the method of expressing the macromolecule in a subject in need, the subject suffers from influenza, parainfluenza, rhinovirus, respiratory syncytial virus, HIV, or other pathogenic bacteria, virus, fungus or parasite. In some alternatives of the method of expressing the macromolecule in a subject in need, the subject suffers from an enzyme deficiency. In some alternatives of the method of expressing the macromolecule in a subject in need, the subject suffers from pulmonary fibrosis. In some alternatives of the method of expressing the macromolecule in a subject in need, the subject suffers from an autoimmune disorder, immune dysregulation or cancer. In some alternatives of the method of expressing the macromolecule in a subject in need, the subject suffers from diabetes. In some alternatives of the method of expressing protein in a subject in need, the subject suffers from hypercholesterolemia. In some alternatives of the method of expressing the macromolecule in a subject in need, the subject is also receiving CAR T-cell therapy. In some alternatives, the plasma cell expresses CD20. In some alternatives, the method further comprises monitoring of patient the subject following plasma cell transplantationadministration. In some alternatives, the subject is determined to no longer need treatment and the method further comprises administering rituxan® or an anti-CD20 antibody for targeted removal of the plasma cells.

In some alternatives, the plasma cell that expresses the macromolecule is used in a therapeutic application such as immunotherapy as envisioned below:

(1) Prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites.

(2) Protein replacement, enzyme replacement and rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia).

(3) Immune modulation via expressed cytokines, cytokine receptors, complement proteins or other inhibitory proteins including, but not limited to: Ill receptor antagonist for the treatment or inhibition of periodic fever/autoinflammatory syndromes; complement inhibitory proteins (including Factor H, Factor I) for the treatment or inhibition of atypical hemolytic uremic syndrome/membranoproliferative glomerulonephritis; and/or C1 inhibitor for hereditary angioedema.

(4) Anti-fibrotic molecules including, but not limited to SCGB1A1 for the treatment or inhibition of pulmonary fibrosis.

(5) Therapeutic antibodies or a binding portion thereof for the treatment or inhibition of autoimmune disorders, autoinflammatory disorders, immune dysregulation and/or cancer including but not limited to: anti-IL-1 monoclonal antibodies or a binding portion thereof for the treatment or inhibition of periodic fever/autoinflammatory syndromes; anti-TNF antibodies or a binding portion thereof for the treatment of or inhibition of inflammatory arthritis/inflammatory bowel disease, anti-IL-33 antibodies or a binding portion thereof for the treatment or inhibition of asthma and/or anti-C5 antibodies or a binding portion thereof for the treatment or inhibition of paroxysmal nocturnal hemoglobinuria/atypical HUS.

(6) Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein).

(7) Glucose responses elements upstream of insulin for the treatment or inhibition of diabetic conditions.

(8) Therapeutic monoclonal antibodies or a binding portion thereof for the treatment or inhibition of hyper-cholesterolemia, including anti-PCSK9 inhibitory antibodies or a binding portion thereof.

In some alternatives of the methods of treating a subject in need or expressing a macromolecule in a subject in need, the subject is administered at least 1-10×10⁶ of edited long-lived plasma cells that express a macromolecule, or a composition, which is used to deliver at least 1-10×10⁶ of edited long-lived plasma cells that express the macromolecule. In some alternatives, the macromolecule is a protein, protein mimetic or a peptide. In some alternatives, the subject receives 1×10⁶, 2×10⁶, 3×10⁶, 4×10⁶, 5×10⁶, 6×10⁶, 7×10⁶, 8×10⁶, 9×10⁶, 10×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹ or 9×10⁹ edited long-lived plasma cells that express a protein or any number of cells in between a range defined by any two aforementioned values. In some alternatives, the plasma cell expresses CD20. In some alternatives, the method further comprises monitoring of patient the subject following plasma cell transplantationadministration. In some alternatives, the subject is determined to no longer need treatment and the method further comprises administering rituxan® or an anti-CD20 antibody for targeted removal of the plasma cells.

### Methods of Ameliorating a Disease in a Subject

A method of ameliorating a disease such as cancer in a subject is also contemplated. The method comprises administering a plasma cell or plasma cell precursor manufactured by any one of the alternative methods provided herein or the plasma cell that expresses a macromolecule manufactured by any one of the alternative methods provided herein, or the composition of any one of the alternative compositions provided herein into the subject in need. In some alternatives, the macromolecule is a protein, protein mimetic or a peptide. In some alternatives, the subject in need suffers from cancer or a subject having cancer is selected or identified to receive an anti-cancer therapy. In some alternatives, the cancer is a solid tumor. In some alternatives, the solid tumor is selected from the group consisting of a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer. In some alternatives, the method further comprises administering a cellular therapy to the subject in need thereof before, after or simultaneous to introducing, providing, or administering any one or more of the cells of any of the alternatives described herein. In some alternatives, the cellular therapy is CAR T-cell therapy. In some alternatives, the plasma cell expresses CD20. In some alternatives, the method further comprises monitoring of patient the subject following plasma cell transplantationadministration. In some alternatives, the subject is determined to no longer need treatment and the method further comprises administering rituxan® or an anti-CD20 antibody for targeted removal of the plasma cells.

The composition comprises the plasma cell manufactured by any one of the alternative methods provided herein or the cell of any one of the alternative cells provided herein. The composition can comprise a plasma cell or plasma cell precursor that expresses a molecule, such as a macromolecule or a plasma cell that is also a long lived plasma cell.

The plasma cell can be manufactured by any one of the alternative methods provided herein. The method of making a plasma cell or plasma cell precursor that expresses a molecule, such as a macromolecule is provided in several alternatives herein. In some alternatives, the macromolecule is a protein, protein mimetic or a peptide. The method comprises isolating B cells, developing the B cells, performing a first round of genome editing of the B cells for protein expression in absence of viral integration, expanding the B cells; and differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the guide sequence comprises a sequence set forth in any one of SEQ ID NO: 2-13, 20-25 or 63-112. In some alternatives, the Cas nuclease comprises Cas1, Cas2, Cas3, Cas4, Cas5, Cas6, Cas7, Cas8 or Cas9. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the step of increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned lengths. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed in a virus, fungus, parasite or bacteria. In some alternatives, the protein comprises an antibody or a binding portion thereof, wherein the antibody or a binding portion thereof is specific for an antigen that is expressed in a viral, fungal, parasitic or bacterial infection. In some alternatives herein, the genome editing is performed by nonpathogenic AAV mediated editing by direct homolougous recombination. In some alternatives herein, the method further comprises purifying the plasma cells of step d), wherein the purifying is performed after the expanding step. In some alternatives herein, the purifying comprises positive selecting of cells against CD138. In some alternatives herein, the selecting the plasma cells is performed by an anti-CD138 antibody for targeted removal of the plasma cells.

In some alternatives, the plasma cell or plasma cell precursor that expresses a macromolecule is a long lived plasma cell. In some alternatives, the macromolecule is a protein, protein mimetic or a peptide. The plasma cell can be manufactured by any one of the alternative methods for making a long lived plasma cell. The method of making a long lived plasma cell, comprises: isolating B cells; activating the B cells; a first round of genome editing of the B cells for protein expression in the absence of viral integration; expanding the B cells; and differentiating the B cells. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, first round of genome editing comprises transducing the B cell with a recombinant adeno-associated virus vector for homologous recombination into a candidate genetic loci. In some alternatives, method further comprises applying methods to prevent somatic hypermutation of an antibody locus and other target loci in the B cells. In some alternatives, the method to prevent somatic hypermutation of an antibody locus in the B cell comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any of the two aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL, and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives herein, the genome editing is performed by nonpathogenic AAV mediated editing by direct homolougous recombination. In some alternatives herein, the method further comprises purifying the plasma cells of step d), wherein the purifying is performed after the expanding step. In some alternatives herein, the purifying comprises positive selecting of cells against CD138. In some alternatives herein, the selecting the plasma cells is performed by an anti-CD138 antibody for targeted removal of the plasma cells.

In some alternatives of the composition, the composition comprises a plasma cell or plasma cell precursor. In some alternatives of the plasma cell, the plasma cell is derived from a B cell. In some alternatives, the B cell comprises B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the plasma cell or plasma cell precursor expresses a macromolecule such as a protein, protein mimetic or a peptide. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell expressing the protein is a long lived plasma cell. In some alternatives herein, the genome editing is performed by nonpathogenic AAV mediated editing by direct homolougous recombination. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection.

In some alternatives of the method of treating or ameliorating a disease in a subject in need, the administering is performed by adoptive transfer. In some alternatives of the method of ameliorating a disease in a subject in need, the subject has received a stem cell administration or solid organ transplantation. In some alternatives of the method of treating or ameliorating a disease in a subject in need, the subject suffers from influenza, parainfluenza, rhinovirus, respiratory syncytial virus, HIV, or other pathogenic bacteria, virus, fungus or parasite. In some alternatives of the method of treating or ameliorating a disease in a subject in need, the subject suffers from an enzyme deficiency. In some alternatives of the method of treating or ameliorating a disease in a subject in need, the subject suffers from pulmonary fibrosis. In some alternatives of the method of treating or ameliorating a disease in a subject in need, the subject suffers from an autoimmune disorder, immune dysregulation or cancer. In some alternatives of the method of treating or ameliorating a disease in a subject in need, the subject suffers from diabetes. In some alternatives of the method of treating or ameliorating a disease in a subject in need, the subject suffers from hypercholesterolemia. In some alternatives of the method of treating or ameliorating a disease in a subject in need, the subject is receiving therapy. In some alternatives of the method of treating or ameliorating a disease in a subject in need, the subject is selected to receive cancer therapy, diabetes therapy, HIV treatment, or viral therapy. In some alternatives of the method of treating or ameliorating a disease in a subject in need, the subject is also receiving CAR T-cell therapy. In some alternatives of the method, the method further comprises monitoring of patient the subject following plasma cell transplantation. In some alternatives, the subject is determined to no longer need treatment and the method further comprises administering rituxan® or an anti-CD20 antibody for targeted removal of the plasma cells.

In some alternatives, the plasma cell or plasma cell precursor that expresses a macromolecule such as a protein, protein mimetic or a peptide is used in a therapeutic application such as immunotherapy as envisioned below:

(1) Prophylactic or therapeutic protection from infection (viral, bacterial, or parasitic) following stem cell administration or solid-organ transplantation in pediatric and adult subjects including, but not limited to neutralizing antibodies that block influenza, parainfluenza, rhinovirus, Respiratory Syncitial Virus (RSV), HIV, pathogenic bacteria, and/or parasites.

(2) Protein replacement, enzyme replacement and rescue of enzyme deficiencies including, but not limited to Factor VIII (Hemophilia A), Factor IX (Hemophilia B), ADAMTS13 (Hereditary TTP), LIPA (lysosomal acid deficiency), SERPING1 (hereditary angioedema), SERPINA1 (alphal anti-trypsin deficiency), GLA (Fabry disease), and/or ALPL (Hypophosphatasia).

(3) Immune modulation via expressed cytokines, cytokine receptors, complement proteins or other inhibitory proteins including, but not limited to: Ill receptor antagonist for the treatment or inhibition of periodic fever/autoinflammatory syndromes; complement inhibitory proteins (including Factor H, Factor I) for the treatment or inhibition of atypical hemolytic uremic syndrome/membranoproliferative glomerulonephritis; and/or C1 inhibitor for hereditary angioedema.

(4) Anti-fibrotic molecules including, but not limited to SCGB1A1 for the treatment or inhibition of pulmonary fibrosis.

(5) Therapeutic antibodies or a binding portion thereof for the treatment or inhibition of autoimmune disorders, autoinflammatory disorders, immune dysregulation and/or cancer including but not limited to: anti-IL-1 monoclonal antibodies or a binding portion thereof for the treatment or inhibition of periodic fever/autoinflammatory syndromes; anti-TNF antibodies or a binding portion thereof for the treatment or inhibition of inflammatory arthritis/inflammatory bowel disease, anti-IL-33 antibodies or a binding portion thereof for the treatment or inhibition of asthma and/or anti-C5 antibodies for the treatment or inhibition of paroxysmal nocturnal hemoglobinuria/atypical HUS.

(6) Anti-thrombotic molecules including, but not limited to APLN to block platelet function. Antithrombotic molecules are further described by Adam et al. (“Apelin: an antithrombotic factor that inhibits platelet function.” Blood. 2016 Feb. 18; 127(7):908-20.; incorporated by reference in its entirety herein).

(7) Glucose responses elements upstream of insulin for the treatment or inhibition of diabetic conditions.

(8) Therapeutic monoclonal antibodies or a binding portion thereof for the treatment or inhibition of hyper-cholesterolemia, including anti-PCSK9 inhibitory antibodies or a binding portion thereof.

In some alternatives of the methods of treating or ameliorating a disease such as cancer in a subject in need or expressing a protein in a subject in need, the subject is administered at least 1-10×10⁶ of edited long-lived plasma cells that express a macromolecule such as a protein, protein mimetic or a peptide, or a composition which is used to deliver at least 1-10×10⁶ of edited long-lived plasma cells that express the macromolecule. In some alternatives, the subject receives 1×10⁶, 2×10⁶, 3×10⁶, 4×10⁶, 5×10⁶, 6×10⁶, 7×10⁶, 8×10⁶, 9×10⁶, 10×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹ or 9×10⁹ edited long-lived plasma cells that express the macromolecule or any number of cells in between a range defined by any two aforementioned values. In some alternatives of the method, the method further comprises monitoring of patient the subject following plasma cell transplantation. In some alternatives, the subject is determined to no longer need treatment and the method further comprises administering rituxan®.

### Methods of Expressing a Protein in a Subject

Methods of expressing a macromolecule, such as a protein, protein mimetic or peptide in a subject in need is provided, wherein the subject is receiving their own gene edited cells as a therapeutic. The method comprises (a) isolating B cells; (b) developing the B cells; (c) performing a first round of genome editing of the B cells for protein expression in absence of viral integration; (d) expanding the B cells; differentiating the B cells, optionally, after step (c) or (d), thereby producing plasma cells that express a protein; and (e) administering the B cells into the subject in need. In some alternatives, the molecule is a macromolecule, such as a protein, protein mimetic or peptide. In some alternatives, the macromolecule is a prodrug. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the developing of the B cells is performed after the B cells are arrested at a specific phase of development and/or activating the B cells such that the B cells are permissive for recombination without further B cell differentiation. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the guide sequence comprises a sequence set forth in any one of SEQ ID NO: 2-13, 20-25 or 63-112. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) performing a second round of genome editing on the B cells to excise a region, further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1X PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/auto-inflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is express in a viral, fungal, parasitic or bacterial infection. In some alternatives, the protein comprises an antibody or a binding portion thereof, wherein the antibody or a binding portion thereof is specific for an antigen that is expressed on a virus, fungus, parasite or bacteria. In some alternatives, the administering is performed by adoptive transfer. In some alternatives, the subject has received a stem cell administration or solid organ transplantation. In some alternatives, the subject suffers from influenza, parainfluenza, rhinovirus, respiratory syncytial virus, HIV, or other pathogenic bacteria, virus, fungus or parasite. In some alternatives, the subject suffers from an enzyme deficiency. In some alternatives, the subject suffers from pulmonary fibrosis. In some alternatives, the subject suffers from an autoimmune disorder, immune dysregulation or cancer. In some alternatives, the subject suffers from diabetes. In some alternatives, the subject suffers from hypercholesterolemia. In some alternatives, the subject is receiving therapy. In some alternatives, the subject is selected or identified to receive a cancer therapy, diabetes therapy, HIV treatment, or viral therapy. In some alternatives, the subject is also receiving CAR T-cell therapy. In some alternatives, the subject in need suffers from cancer or a subject having cancer is selected or identified to receive an anti-cancer therapy. In some alternatives, the cancer is a solid tumor. In some alternatives, the solid tumor is selected from the group consisting of a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer. In some alternatives, the method further comprises administering a cellular therapy to the subject in need thereof before, after or simultaneous to introducing, providing, or administering any one or more of the cells of any of the alternatives described herein. In some alternatives, the cellular therapy is CAR T-cell therapy. In some alternatives herein, the genome editing is performed by nonpathogenic AAV mediated editing by direct homolougous recombination. In some alternatives, the method further comprises purifying the plasma cells. In some alternatives, the purifying comprises positive selecting of cells against CD138. In some alternatives, the selecting the plasma cells is performed by an anti-CD138 antibody for targeted removal of the plasma cells. In some alternatives, the purifying comprises using anti-CD138 beads for plasma cell selection.

In some alternatives of the methods of treating a subject in need for a disease such as cancer or expressing a macromolecule, such as a protein, protein mimetic or a peptide in a subject in need, the subject is administered at least 1-10×10⁶ of edited long-lived plasma cells that express the macromolecule, or a composition which is used to deliver at least 1-10×10⁶ of edited long-lived plasma cells that express a protein. In some alternatives, the subject receives 1×10⁶, 2×10⁶, 3×10⁶, 4×10⁶, 5×10⁶, 6×10⁶, 7×10⁶, 8×10⁶, 9×10⁶, 10×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10, 1×10⁹, 2×10⁹, 3×10⁹, 4×10⁹, 5×10⁹, 6×10⁹, 7×10⁹, 8×10⁹ or 9×10⁹ edited long-lived plasma cells that express a protein or any number of cells in between a range defined by any two aforementioned values. In some alternatives, the method further comprise monitoring of patient the subject following plasma cell transplantation. In some alternatives, the subject is determined to no longer need treatment and the method further comprises administering rituxan® or an anti-CD20 antibody for targeted removal of the plasma cells.

### Targeting and Sequencing Methods to Ensure the Integrity of the Terminal B Cell Product

Methods are further contemplated to further assess the B cell product that is manufactured by the alternative methods herein. These methods are used prior to adding the cells to the composition or delivery of the cells to a subject in need.

Samples of the product cells, such as a long lived plasma cells or plasma cells that are expressing protein are used for DNA extraction to assay for disruption at their targeted sequences. Additionally, the cells are further tested for expression of specific proteins.

Developing a deep and targeted sequencing method to ensure the integrity of the terminal B cell product at a loci known to be modified by AID and/or confer susceptibility to non-Hodgkin's lymphoma or multiple myeloma is also proposed. To do this microarrays coupled to DNA oligos will be developed and used to enrich for exonic and genomic DNA flanking genes known to have mutations associated with non-Hodgkin's lymphoma or myeloma (˜500 genes). To assess the integrity of the product, genomic DNA from the product will be captured and use high-throughput sequencing with detailed coverage (>1000×) to determine if mutations are present.

### Additional Sequences can be Used for the Described Alternatives:

Guide RNA sequences

| Gene | Sequence |
|---|---|
| PAX5 | UGU GAA UGG ACG GCC ACU CC (SEQ ID NO: 2) |
| PAX5 | UGU AGU CCG CCA GAG GAU AG (SEQ ID NO: 3) |
| IRF8 | AUU GAC AGU AGC AUG UAU CC (SEQ ID NO: 4) |
| IRF8 | CGG AAA UGU CCA GUU GGG AC (SEQ ID NO: 5) |
| BACH2 | GUU CCU GCG CAU GCA CAA CC (SEQ ID NO: 6) |
| BACH2 | CUG UGA CGU GAC UUU GAU CG (SEQ ID NO: 7) |
| CCR5 | CAA UGU GUC AAC UCU UGA CA (SEQ ID NO: 8) |
| CCR5 | GCU GUG UUU GCG UCU CUC CC (SEQ ID NO: 9) |
| CARD11 | CAAUGACCUUACACUGACGC (SEQ ID NO: 10) |
| PRDM1 | UGAUGGCGGUACUUCGGUUC (SEQ ID NO: 11) |
| PRDM1 | AGGAUGCGGAUAUGACUCUG (SEQ ID NO: 12) |
| PRDM1 | GGGGAGCGAGUGAUGUACGU (SEQ ID NO: 13) |

AAV repair template sequence list (sequences below)

Construct Name
1079_pscAAV-MND.GFP
1347_pscAAV.Blimp.0.4kb.MND.GFP
1348_pAAV.Blimp.0.4kb.MND.GFP.pA
1361_pAAV.Blimp1.1.0kb.MND.GFP.pA
1366_CCR5.MND.BAFF_CRISPR.HR
1367_CCR5.MND.BAFF.2A.GFP
1378_CCR5.MND.mCherry.2A.coFIXpadua.WPRE.pa
1376_CCR5.MND.FiX.coFIXpadua.WPRE.pA

| 1079_pscAAV-MND.GFP | |
|---|---|
| (SEQ ID NO: 51) | |
| ORIGIN | |
| 1 | aagcttcccg gggggatctg ggccactccc tctctgcgcg ctcgctcgct cactgaggcc |
| 61 | gggcgaccaa aggtcgcccg acgcccgggc tttgcccggg cggcctcagt gagcgagcga |
| 121 | gcgcgcagag agggagtggc caactccatc actaggggtt cctggagggg tggagtcgtg |
| 181 | acctagggaa cagagaaaca ggagaatatg ggccaaacag gatatctgtg gtaagcagtt |
| 241 | cctgccccgg ctcagggcca agaacagttg gaacagcaga atatgggcca aacaggatat |
| 301 | ctgtggtaag cagttcctgc cccggctcag ggccaagaac agatggtccc cagatgcggt |
| 361 | cccgccctca gcagtttcta gagaaccatc agatgtttcc agggtgcccc aaggacctga |
| 421 | aatgaccctg tgccttattt gaactaacca atcagttcgc ttctcgcttc tgttcgcgcg |
| 481 | cttctgctcc ccgagctcta tataagcaga gctcgtttag tgaaccgtca gatcgcctgg |
| 541 | agacgccatc cacgctgttt tgacttccat agaaggatcc tcgaggccac catggtgagc |
| 601 | aagggcgagg agctgttcac cggggtggtg cccatcctgg tcgagctgga cggcgacgta |
| 661 | aacggccaca agttcagcgt gtccggcgag ggcgagggcg atgccaccta cggcaagctg |
| 721 | accctgaagt tcatctgcac caccggcaag ctgcccgtgc cctggcccac cctcgtgacc |
| 781 | accctgacct acggcgtgca gtgcttcagc cgctaccccg accacatgaa gcagcacgac |
| 841 | ttcttcaagt ccgccatgcc cgaaggctac gtccaggagc gcaccatctt cttcaaggac |
| 901 | gacggcaact acaagacccg cgccgaggtg aagttcgagg gcgacaccct ggtgaaccgc |
| 961 | atcgagctga agggcatcga cttcaaggag gacggcaaca tcctggggca caagctggag |
| 1021 | tacaactaca acagccacaa cgtctatatc atggccgaca agcagaagaa cggcatcaag |
| 1081 | gtgaacttca agatccgcca caacatcgag gacggcagcg tgcagctcgc cgaccactac |
| 1141 | cagcagaaca cccccatcgg cgacggcccc gtgctgctgc ccgacaacca ctacctgagc |
| 1201 | acccagtccg ccctgagcaa agaccccaac gagaagcgcg atcacatggt cctgctggag |
| 1261 | ttcgtgaccg ccgccgggat cactctcggc atggacgagc tgtacaagta agcggccgca |
| 1321 | attcacccca ccagtgcagg ctgcctatca gaaagtggtg gctggtgtgg ctaatgccct |
| 1381 | ggcccacaag tatcactaag ctcgctttct tgctgtccaa tttctattaa aggttccttt |
| 1441 | gttccctaag tccaactact aaactggggg atattatgaa gggccttgag catctggatt |
| 1501 | ctgcctaata aaaaacattt attttcattg caatgatgta tttaaattat ttctgaatat |
| 1561 | tttactaaaa agggaatgtg ggaggtcagt gcatttaaaa cataaagaaa tgaagagcta |
| 1621 | gttcaaacct tgggaaaata cactatatct taaactccat gaaagaaggt gaggctgcaa |
| 1681 | acagctaatg cacattggca acagcccctg atgcctatgc cttattcatc cctcagaaaa |
| 1741 | ggattcaagt agaggcttga tttggaggtt aaagttttgc tatgctgtat tttacattac |
| 1801 | ttattgtttt agctgtcctc atgaatgtct tttcactacc catttgctta tcctgcatct |
| 1861 | ctcagccttg actccactca gttctcttgc ttagagatac cacctttccc ctgaagtgtt |
| 1921 | ccttccatgt tttacggcga gatggtttct cctcgcctgg ccactcagcc ttagttgtct |
| 1981 | ctgttgtctt atagaggtct acttgaagaa ggaaaaacag ggggcatggt ttgactgtcc |
| 2041 | tgtgagccct tcttccctgc ctcccccact cacagtgaca ctagtccact ccctctctgc |
| 2101 | gcgctcgctc gctcactgag gccgggcgac caaaggtcgc ccgacgcccg ggctttgccc |
| 2161 | gggcggcctc agtgagcgag cgagcgcgca gagagggaca gatccgggcc cgcatgcgtc |
| 2221 | gacaattcac tggccgtcgt tttacaacgt cgtgactggg aaaaccctgg cgttacccaa |
| 2281 | cttaatcgcc ttgcagcaca tccccctttc gccagctggc gtaatagcga agaggcccgc |
| 2341 | accgatcgcc cttcccaaca gttgcgcagc ctgaatggcg aatggcgcct gatgcggtat |
| 2401 | tttctcctta cgcatctgtg cggtatttca caccgcatat ggtgcactct cagtacaatc |
| 2461 | tgctctgatg ccgcatagtt aagccagccc cgacacccgc caacacccgc tgacgcgccc |
| 2521 | tgacgggctt gtctgctccc ggcatccgct tacagacaag ctgtgaccgt ctccgggagc |
| 2581 | tgcatgtgtc agaggttttc accgtcatca ccgaaacgcg cgagacgaaa gggcctcgtg |
| 2641 | atacgcctat ttttataggt taatgtcatg ataataatgg tttcttagac gtcaggtggc |
| 2701 | acttttcggg gaaatgtgcg cggaacccct atttgtttat ttttctaaat acattcaaat |
| 2761 | atgtatccgc tcatgagaca ataaccctga taaatgcttc aataatattg aaaaaggaag |
| 2821 | agtatgagta ttcaacattt ccgtgtcgcc cttattccct tttttgcggc attttgcctt |
| 2881 | cctgtttttg ctcacccaga aacgctggtg aaagtaaaag atgctgaaga tcagttgggt |
| 2941 | gcacgagtgg gttacatcga actggatctc aacagcggta agatccttga gagttttcgc |
| 3001 | cccgaagaac gttttccaat gatgagcact tttaaagttc tgctatgtgg cgcggtatta |
| 3061 | tcccgtattg acgccgggca agagcaactc ggtcgccgca tacactattc tcagaatgac |
| 3121 | ttggttgagt actcaccagt cacagaaaag catcttacgg atggcatgac agtaagagaa |
| 3181 | ttatgcagtg ctgccataac catgagtgat aacactgcgg ccaacttact tctgacaacg |
| 3241 | atcggaggac cgaaggagct aaccgctttt ttgcacaaca tgggggatca tgtaactcgc |
| 3301 | cttgatcgtt gggaaccgga gctgaatgaa gccataccaa acgacgagcg tgacaccacg |
| 3361 | atgcctgtag caatggcaac aacgttgcgc aaactattaa ctggcgaact acttactcta |
| 3421 | gcttcccggc aacaattaat agactggatg gaggcggata aagttgcagg accacttctg |
| 3481 | cgctcggccc ttccggctgg ctggtttatt gctgataaat ctggagccgg tgagcgtggg |
| 3541 | tctcgcggta tcattgcagc actggggcca gatggtaagc cctcccgtat cgtagttatc |
| 3601 | tacacgacgg ggagtcaggc aactatggat gaacgaaata gacagatcgc tgagataggt |
| 3661 | gcctcactga ttaagcattg gtaactgtca gaccaagttt actcatatat actttagatt |
| 3721 | gatttaaaac ttcattttta atttaaaagg atctaggtga agatcctttt tgataatctc |
| 3781 | atgaccaaaa tcccttaacg tgagttttcg ttccactgag cgtcagaccc cgtagaaaag |
| 3841 | atcaaaggat cttcttgaga tccttttttt ctgcgcgtaa tctgctgctt gcaaacaaaa |
| 3901 | aaaccaccgc taccagcggt ggtttgtttg ccggatcaag agctaccaac tctttttccg |
| 3961 | aaggtaactg gcttcagcag agcgcagata ccaaatactg ttcttctagt gtagccgtag |
| 4021 | ttaggccacc acttcaagaa ctctgtagca ccgcctacat acctcgctct gctaatcctg |
| 4081 | ttaccagtgg ctgctgccag tggcgataag tcgtgtctta ccgggttgga ctcaagacga |
| 4141 | tagttaccgg ataaggcgca gcggtcgggc tgaacggggg gttcgtgcac acagcccagc |
| 4201 | ttggagcgaa cgacctacac cgaactgaga tacctacagc gtgagctatg agaaagcgcc |
| 4261 | acgcttcccg aagggagaaa ggcggacagg tatccggtaa gcggcagggt cggaacagga |
| 4321 | gagcgcacga gggagcttcc agggggaaac gcctggtatc tttatagtcc tgtcgggttt |
| 4381 | cgccacctct gacttgagcg tcgatttttg tgatgctcgt caggggggcg gagcctatgg |
| 4441 | aaaaacgcca gcaacgcggc ctttttacgg ttcctggcct tttgctggcc ttttgctcac |
| 4501 | atgttctttc ctgcgttatc ccctgattct gtggataacc gtattaccgc ctttgagtga |
| 4561 | gctgataccg ctcgccgcag ccgaacgacc gagcgcagcg agtcagtgag cgaggaagcg |
| 4621 | gaagagcgcc caatacgcaa accgcctctc cccgcgcgtt ggccgattca ttaatgcagc |
| 4681 | tggcacgaca ggtttcccga ctggaaagcg ggcagtgagc gcaacgcaat taatgtgagt |
| 4741 | tagctcactc attaggcacc ccaggcttta cactttatgc ttccggctcg tatgttgtgt |
| 4801 | ggaattgtga gcggataaca atttcacaca ggaaacagct atgaccatga ttacgccaag |
| 4861 | ctctcgagat ctaga |
| 1347_pscAAV.Blimp.0.4 kb.MND.GFP | |
| (SEQ ID NO: 52) | |
| LOCUS (#1347)\pscAAV.B 5056 bp DNA circular 10-MAR.-2017 | |
| DEFINITION File from St. Jude Vector Lab. | |
| ACCESSION urn.local . . . 2c-6onxobo | |
| ORIGIN | |
| 1 | aagcttcccg gggggatctg ggccactccc tctctgcgcg ctcgctcgct cactgaggcc |
| 61 | gggcgaccaa aggtcgcccg acgcccgggc tttgcccggg cggcctcagt gagcgagcga |
| 121 | gcgcgcagag agggagtggc caactccatc actaggggtt cctggagggg tggagtcgtg |
| 181 | acctaggacg cgtgccagct gttactcagg ttttctcaag aaggaggagc aactttggca |
| 241 | gttttgcttc agttctctct agccctctgt gtaatcgccc ctttttcttt atttcagcac |
| 301 | aaacacagag cagtctaaag caaccgagca ctgagaaaaa tgaactctgc ccaaagaatg |
| 361 | tcccaaagag agagtacagc gtgaaagaaa tcctaaaatt ggactccaac ccctccaaag |
| 421 | gaaaggacct ctaccgttct aacatttcac ccctcacatc agaaaaggac ctcgatgact |
| 481 | ttagaagacg tgggagcccc gaaatgccct tctaccctcg ggtcgtttac cccatccggg |
| 541 | cccctctgcc agaagacttt ttgaaagctt ccctggccta cgggatcgag agagaacaga |
| 601 | gaaacaggag aatatgggcc aaacaggata tctgtggtaa gcagttcctg ccccggctca |
| 661 | gggccaagaa cagttggaac agcagaatat gggccaaaca ggatatctgt ggtaagcagt |
| 721 | tcctgccccg gctcagggcc aagaacagat ggtccccaga tgcggtcccg ccctcagcag |
| 781 | tttctagaga accatcagat gtttccaggg tgccccaagg acctgaaatg accctgtgcc |
| 841 | ttatttgaac taaccaatca gttcgcttct cgcttctgtt cgcgcgcttc tgctccccga |
| 901 | gctctatata agcagagctc gtttagtgaa ccgtcagatc gcctggagac gccatccacg |
| 961 | ctgttttgac ttccatagaa ggatctcgag gccaccatgg tgagcaaggg cgaggagctg |
| 1021 | ttcaccgggg tggtgcccat cctggtcgag ctggacggcg acgtaaacgg ccacaagttc |
| 1081 | agcgtgtccg gcgagggcga gggcgatgcc acctacggca agctgaccct gaagttcatc |
| 1141 | tgcaccaccg gcaagctgcc cgtgccctgg cccaccctcg tgaccaccct gacctacggc |
| 1201 | gtgcagtgct tcagccgcta ccccgaccac atgaagcagc acgacttctt caagtccgcc |
| 1261 | atgcccgaag gctacgtcca ggagcgcacc atcttcttca aggacgacgg caactacaag |
| 1321 | acccgcgccg aggtgaagtt cgagggcgac accctggtga accgcatcga gctgaagggc |
| 1381 | atcgacttca aggaggacgg caacatcctg gggcacaagc tggagtacaa ctacaacagc |
| 1441 | cacaacgtct atatcatggc cgacaagcag aagaacggca tcaaggtgaa cttcaagatc |
| 1501 | cgccacaaca tcgaggacgg cagcgtgcag ctcgccgacc actaccagca gaacaccccc |
| 1561 | atcggcgacg gccccgtgct gctgcccgac aaccactacc tgagcaccca gtccgccctg |
| 1621 | agcaaagacc ccaacgagaa gcgcgatcac atggtcctgc tggagttcgt gaccgccgcc |
| 1681 | gggatcactc tcggcatgga cgagctgtac aagtaaacta gtgtcgactg ctttatttgt |
| 1741 | gaaatttgtg atgctattgc tttatttgta accattataa gctgcaataa acaagttaac |
| 1801 | aacaacaatt gcattcattt tatgtttcag gttcaggggg aggtgtggga ggttttttaa |
| 1861 | aacgtacatc actcgctccc ccattccatc ctccaccact ccaagcccct ctgcaagaag |
| 1921 | cagccccgac caaagcctca agagctccag ccctcacagc agccctggga atacggtgtc |
| 1981 | ccctgtgggc cccggctctc aagagcaccg ggactcctac gcttacttga acgcgtccta |
| 2041 | cggcacggaa ggtttgggct cctaccctgg ctacgcaccc ctgccccacc tcccgccagc |
| 2101 | tttcatcccc tcgtacaacg ctcactaccc caagttcctc ttgcccccct acggcatgaa |
| 2161 | ttgtaatggc ctgagcgctg tgagcagcat gaatggcatc aacaactttg gcctcttccc |
| 2221 | gaggctgtgc cctgtctaca gcaatctcct cggtgggggc actagtccac tccctctctg |
| 2281 | cgcgctcgct cgctcactga ggccgggcga ccaaaggtcg cccgacgccc gggctttgcc |
| 2341 | cgggcggcct cagtgagcga gcgagcgcgc agagagggac agatccgggc ccgcatgcgt |
| 2401 | cgacaattca ctggccgtcg ttttacaacg tcgtgactgg gaaaaccctg gcgttaccca |
| 2461 | acttaatcgc cttgcagcac atcccccttt cgccagctgg cgtaatagcg aagaggcccg |
| 2521 | caccgatcgc ccttcccaac agttgcgcag cctgaatggc gaatggcgcc tgatgcggta |
| 2581 | ttttctcctt acgcatctgt gcggtatttc acaccgcata tggtgcactc tcagtacaat |
| 2641 | ctgctctgat gccgcatagt taagccagcc ccgacacccg ccaacacccg ctgacgcgcc |
| 2701 | ctgacgggct tgtctgctcc cggcatccgc ttacagacaa gctgtgaccg tctccgggag |
| 2761 | ctgcatgtgt cagaggtttt caccgtcatc accgaaacgc gcgagacgaa agggcctcgt |
| 2821 | gatacgccta tttttatagg ttaatgtcat gataataatg gtttcttaga cgtcaggtgg |
| 2881 | cacttttcgg ggaaatgtgc gcggaacccc tatttgttta tttttctaaa tacattcaaa |
| 2941 | tatgtatccg ctcatgagac aataaccctg ataaatgctt caataatatt gaaaaaggaa |
| 3001 | gagtatgagt attcaacatt tccgtgtcgc ccttattccc ttttttgcgg cattttgcct |
| 3061 | tcctgttttt gctcacccag aaacgctggt gaaagtaaaa gatgctgaag atcagttggg |
| 3121 | tgcacgagtg ggttacatcg aactggatct caacagcggt aagatccttg agagttttcg |
| 3181 | ccccgaagaa cgttttccaa tgatgagcac ttttaaagtt ctgctatgtg gcgcggtatt |
| 3241 | atcccgtatt gacgccgggc aagagcaact cggtcgccgc atacactatt ctcagaatga |
| 3301 | cttggttgag tactcaccag tcacagaaaa gcatcttacg gatggcatga cagtaagaga |
| 3361 | attatgcagt gctgccataa ccatgagtga taacactgcg gccaacttac ttctgacaac |
| 3421 | gatcggagga ccgaaggagc taaccgcttt tttgcacaac atgggggatc atgtaactcg |
| 3481 | ccttgatcgt tgggaaccgg agctgaatga agccatacca aacgacgagc gtgacaccac |
| 3541 | gatgcctgta gcaatggcaa caacgttgcg caaactatta actggcgaac tacttactct |
| 3601 | agcttcccgg caacaattaa tagactggat ggaggcggat aaagttgcag gaccacttct |
| 3661 | gcgctcggcc cttccggctg gctggtttat tgctgataaa tctggagccg gtgagcgtgg |
| 3721 | gtctcgcggt atcattgcag cactggggcc agatggtaag ccctcccgta tcgtagttat |
| 3781 | ctacacgacg gggagtcagg caactatgga tgaacgaaat agacagatcg ctgagatagg |
| 3841 | tgcctcactg attaagcatt ggtaactgtc agaccaagtt tactcatata tactttagat |
| 3901 | tgatttaaaa cttcattttt aatttaaaag gatctaggtg aagatccttt ttgataatct |
| 3961 | catgaccaaa atcccttaac gtgagttttc gttccactga gcgtcagacc ccgtagaaaa |
| 4021 | gatcaaagga tcttcttgag atcctttttt tctgcgcgta atctgctgct tgcaaacaaa |
| 4081 | aaaaccaccg ctaccagcgg tggtttgttt gccggatcaa gagctaccaa ctctttttcc |
| 4141 | gaaggtaact ggcttcagca gagcgcagat accaaatact gttcttctag tgtagccgta |
| 4201 | gttaggccac cacttcaaga actctgtagc accgcctaca tacctcgctc tgctaatcct |
| 4261 | gttaccagtg gctgctgcca gtggcgataa gtcgtgtctt accgggttgg actcaagacg |
| 4321 | atagttaccg gataaggcgc agcggtcggg ctgaacgggg ggttcgtgca cacagcccag |
| 4381 | cttggagcga acgacctaca ccgaactgag atacctacag cgtgagctat gagaaagcgc |
| 4441 | cacgcttccc gaagggagaa aggcggacag gtatccggta agcggcaggg tcggaacagg |
| 4501 | agagcgcacg agggagcttc cagggggaaa cgcctggtat ctttatagtc ctgtcgggtt |
| 4561 | tcgccacctc tgacttgagc gtcgattttt gtgatgctcg tcaggggggc ggagcctatg |
| 4621 | gaaaaacgcc agcaacgcgg cctttttacg gttcctggcc ttttgctggc cttttgctca |
| 4681 | catgttcttt cctgcgttat cccctgattc tgtggataac cgtattaccg cctttgagtg |
| 4741 | agctgatacc gctcgccgca gccgaacgac cgagcgcagc gagtcagtga gcgaggaagc |
| 4801 | ggaagagcgc ccaatacgca aaccgcctct ccccgcgcgt tggccgattc attaatgcag |
| 4861 | ctggcacgac aggtttcccg actggaaagc gggcagtgag cgcaacgcaa ttaatgtgag |
| 4921 | ttagctcact cattaggcac cccaggcttt acactttatg cttccggctc gtatgttgtg |
| 4981 | tggaattgtg agcggataac aatttcacac aggaaacagc tatgaccatg attacgccaa |
| 5041 | gctctcgaga tctaga |
| 1348_pAAV.Blimp.0.4 kb.MND.GFP.pA | |
| (SEQ ID NO: 53) | |
| LOCUS (#1348)\pAAV.Bli 6006 bp DNA circular UNA 10-MAR.-2017 | |
| DEFINITION GFP in AAV, via MD expression cassette, ie, CMV, globin intron, | |
| GFP, globin polyA. This plasmid sequence was corrected on | |
| Oct. 3, 2002, with correction record #201. AAV non-vector error, | |
| probably present in all AAV constructs. Merely deletes 3 bp prior | |
| to left ITR sequence. Replacement of CAGCAGCTGC GCGCTCGCTC GCTCA | |
| with CAGCTGCGCG CTCGCTCGCT CA. | |
| ACCESSION urn.local . . . e-6i3n6r4 | |
| VERSION urn.local . . . e-6i3n6r4 | |
| KEYWORDS . | |
| SOURCE | |
| ORGANISM | |
| . | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac acgcgtgcca |
| 181 | gctgttactc aggttttctc aagaaggagg agcaactttg gcagttttgc ttcagttctc |
| 241 | tctagccctc tgtgtaatcg cccctttttc tttatttcag cacaaacaca gagcagtcta |
| 301 | aagcaaccga gcactgagaa aaatgaactc tgcccaaaga atgtcccaaa gagagagtac |
| 361 | agcgtgaaag aaatcctaaa attggactcc aacccctcca aaggaaagga cctctaccgt |
| 421 | tctaacattt cacccctcac atcagaaaag gacctcgatg actttagaag acgtgggagc |
| 481 | cccgaaatgc ccttctaccc tcgggtcgtt taccccatcc gggcccctct gccagaagac |
| 541 | tttttgaaag cttccctggc ctacgggatc gagagagaac agagaaacag gagaatatgg |
| 601 | gccaaacagg atatctgtgg taagcagttc ctgccccggc tcagggccaa gaacagttgg |
| 661 | aacagcagaa tatgggccaa acaggatatc tgtggtaagc agttcctgcc ccggctcagg |
| 721 | gccaagaaca gatggtcccc agatgcggtc ccgccctcag cagtttctag agaaccatca |
| 781 | gatgtttcca gggtgcccca aggacctgaa atgaccctgt gccttatttg aactaaccaa |
| 841 | tcagttcgct tctcgcttct gttcgcgcgc ttctgctccc cgagctctat ataagcagag |
| 901 | ctcgtttagt gaaccgtcag atcgcctgga gacgccatcc acgctgtttt gacttccata |
| 961 | gaaggatctc gaggccacca tggtgagcaa gggcgaggag ctgttcaccg gggtggtgcc |
| 1021 | catcctggtc gagctggacg gcgacgtaaa cggccacaag ttcagcgtgt ccggcgaggg |
| 1081 | cgagggcgat gccacctacg gcaagctgac cctgaagttc atctgcacca ccggcaagct |
| 1141 | gcccgtgccc tggcccaccc tcgtgaccac cctgacctac ggcgtgcagt gcttcagccg |
| 1201 | ctaccccgac cacatgaagc agcacgactt cttcaagtcc gccatgcccg aaggctacgt |
| 1261 | ccaggagcgc accatcttct tcaaggacga cggcaactac aagacccgcg ccgaggtgaa |
| 1321 | gttcgagggc gacaccctgg tgaaccgcat cgagctgaag ggcatcgact tcaaggagga |
| 1381 | cggcaacatc ctggggcaca agctggagta caactacaac agccacaacg tctatatcat |
| 1441 | ggccgacaag cagaagaacg gcatcaaggt gaacttcaag atccgccaca acatcgagga |
| 1501 | cggcagcgtg cagctcgccg accactacca gcagaacacc cccatcggcg acggccccgt |
| 1561 | gctgctgccc gacaaccact acctgagcac ccagtccgcc ctgagcaaag accccaacga |
| 1621 | gaagcgcgat cacatggtcc tgctggagtt cgtgaccgcc gccgggatca ctctcggcat |
| 1681 | ggacgagctg tacaagtaaa ctagtgtcga ctgctttatt tgtgaaattt gtgatgctat |
| 1741 | tgctttattt gtaaccatta taagctgcaa taaacaagtt aacaacaaca attgcattca |
| 1801 | ttttatgttt caggttcagg gggaggtgtg ggaggttttt taaaacgtac atcactcgct |
| 1861 | cccccattcc atcctccacc actccaagcc cctctgcaag aagcagcccc gaccaaagcc |
| 1921 | tcaagagctc cagccctcac agcagccctg ggaatacggt gtcccctgtg ggccccggct |
| 1981 | ctcaagagca ccgggactcc tacgcttact tgaacgcgtc ctacggcacg gaaggtttgg |
| 2041 | gctcctaccc tggctacgca cccctgcccc acctcccgcc agctttcatc ccctcgtaca |
| 2101 | acgctcacta ccccaagttc ctcttgcccc cctacggcat gaattgtaat ggcctgagcg |
| 2161 | ctgtgagcag catgaatggc atcaacaact ttggcctctt cccgaggctg tgccctgtct |
| 2221 | acagcaatct cctcggtggg ggcatctaga gtagataagt agcatggcgg gttaatcatt |
| 2281 | aactacaagg aacccctagt gatggagttg gccactccct ctctgcgcgc tcgctcgctc |
| 2341 | actgaggccg ggcgaccaaa ggtcgcccga cgcccgggct ttgcccgggc ggcctcagtg |
| 2401 | agcgagcgag cgcgccagct ggcgtaatag cgaagaggcc cgcaccgatc gcccttccca |
| 2461 | acagttgcgc agcctgaatg gcgaatggcg attccgttgc aatggctggc ggtaatattg |
| 2521 | ttctggatat taccagcaag gccgatagtt tgagttcttc tactcaggca agtgatgtta |
| 2581 | ttactaatca aagaagtatt gcgacaacgg ttaatttgcg tgatggacag actcttttac |
| 2641 | tcggtggcct cactgattat aaaaacactt ctcaggattc tggcgtaccg ttcctgtcta |
| 2701 | aaatcccttt aatcggcctc ctgtttagct cccgctctga ttctaacgag gaaagcacgt |
| 2761 | tatacgtgct cgtcaaagca accatagtac gcgccctgta gcggcgcatt aagcgcggcg |
| 2821 | ggtgtggtgg ttacgcgcag cgtgaccgct acacttgcca gcgccctagc gcccgctcct |
| 2881 | ttcgctttct tcccttcctt tctcgccacg ttcgccggct ttccccgtca agctctaaat |
| 2941 | cgggggctcc ctttagggtt ccgatttagt gctttacggc acctcgaccc caaaaaactt |
| 3001 | gattagggtg atggttcacg tagtgggcca tcgccctgat agacggtttt tcgccctttg |
| 3061 | acgttggagt ccacgttctt taatagtgga ctcttgttcc aaactggaac aacactcaac |
| 3121 | cctatctcgg tctattcttt tgatttataa gggattttgc cgatttcggc ctattggtta |
| 3181 | aaaaatgagc tgatttaaca aaaatttaac gcgaatttta acaaaatatt aacgtttaca |
| 3241 | atttaaatat ttgcttatac aatcttcctg tttttggggc ttttctgatt atcaaccggg |
| 3301 | gtacatatga ttgacatgct agttttacga ttaccgttca tcgattctct tgtttgctcc |
| 3361 | agactctcag gcaatgacct gatagccttt gtagagacct ctcaaaaata gctaccctct |
| 3421 | ccggcatgaa tttatcagct agaacggttg aatatcatat tgatggtgat ttgactgtct |
| 3481 | ccggcctttc tcacccgttt gaatctttac ctacacatta ctcaggcatt gcatttaaaa |
| 3541 | tatatgaggg ttctaaaaat ttttatcctt gcgttgaaat aaaggcttct cccgcaaaag |
| 3601 | tattacaggg tcataatgtt tttggtacaa ccgatttagc tttatgctct gaggctttat |
| 3661 | tgcttaattt tgctaattct ttgccttgcc tgtatgattt attggatgtt ggaatcgcct |
| 3721 | gatgcggtat tttctcctta cgcatctgtg cggtatttca caccgcatat ggtgcactct |
| 3781 | cagtacaatc tgctctgatg ccgcatagtt aagccagccc cgacacccgc caacacccgc |
| 3841 | tgacgcgccc tgacgggctt gtctgctccc ggcatccgct tacagacaag ctgtgaccgt |
| 3901 | ctccgggagc tgcatgtgtc agaggttttc accgtcatca ccgaaacgcg cgagacgaaa |
| 3961 | gggcctcgtg atacgcctat ttttataggt taatgtcatg ataataatgg tttcttagac |
| 4021 | gtcaggtggc acttttcggg gaaatgtgcg cggaacccct atttgtttat ttttctaaat |
| 4081 | acattcaaat atgtatccgc tcatgagaca ataaccctga taaatgcttc aataatattg |
| 4141 | aaaaaggaag agtatgagta ttcaacattt ccgtgtcgcc cttattccct tttttgcggc |
| 4201 | attttgcctt cctgtttttg ctcacccaga aacgctggtg aaagtaaaag atgctgaaga |
| 4261 | tcagttgggt gcacgagtgg gttacatcga actggatctc aacagcggta agatccttga |
| 4321 | gagttttcgc cccgaagaac gttttccaat gatgagcact tttaaagttc tgctatgtgg |
| 4381 | cgcggtatta tcccgtattg acgccgggca agagcaactc ggtcgccgca tacactattc |
| 4441 | tcagaatgac ttggttgagt actcaccagt cacagaaaag catcttacgg atggcatgac |
| 4501 | agtaagagaa ttatgcagtg ctgccataac catgagtgat aacactgcgg ccaacttact |
| 4561 | tctgacaacg atcggaggac cgaaggagct aaccgctttt ttgcacaaca tgggggatca |
| 4621 | tgtaactcgc cttgatcgtt gggaaccgga gctgaatgaa gccataccaa acgacgagcg |
| 4681 | tgacaccacg atgcctgtag caatggcaac aacgttgcgc aaactattaa ctggcgaact |
| 4741 | acttactcta gcttcccggc aacaattaat agactggatg gaggcggata aagttgcagg |
| 4801 | accacttctg cgctcggccc ttccggctgg ctggtttatt gctgataaat ctggagccgg |
| 4861 | tgagcgtggg tctcgcggta tcattgcagc actggggcca gatggtaagc cctcccgtat |
| 4921 | cgtagttatc tacacgacgg ggagtcaggc aactatggat gaacgaaata gacagatcgc |
| 4981 | tgagataggt gcctcactga ttaagcattg gtaactgtca gaccaagttt actcatatat |
| 5041 | actttagatt gatttaaaac ttcattttta atttaaaagg atctaggtga agatcctttt |
| 5101 | tgataatctc atgaccaaaa tcccttaacg tgagttttcg ttccactgag cgtcagaccc |
| 5161 | cgtagaaaag atcaaaggat cttcttgaga tccttttttt ctgcgcgtaa tctgctgctt |
| 5221 | gcaaacaaaa aaaccaccgc taccagcggt ggtttgtttg ccggatcaag agctaccaac |
| 5281 | tctttttccg aaggtaactg gcttcagcag agcgcagata ccaaatactg tccttctagt |
| 5341 | gtagccgtag ttaggccacc acttcaagaa ctctgtagca ccgcctacat acctcgctct |
| 5401 | gctaatcctg ttaccagtgg ctgctgccag tggcgataag tcgtgtctta ccgggttgga |
| 5461 | ctcaagacga tagttaccgg ataaggcgca gcggtcgggc tgaacggggg gttcgtgcac |
| 5521 | acagcccagc ttggagcgaa cgacctacac cgaactgaga tacctacagc gtgagctatg |
| 5581 | agaaagcgcc acgcttcccg aagggagaaa ggcggacagg tatccggtaa gcggcagggt |
| 5641 | cggaacagga gagcgcacga gggagcttcc agggggaaac gcctggtatc tttatagtcc |
| 5701 | tgtcgggttt cgccacctct gacttgagcg tcgatttttg tgatgctcgt caggggggcg |
| 5761 | gagcctatgg aaaaacgcca gcaacgcggc ctttttacgg ttcctggcct tttgctggcc |
| 5821 | ttttgctcac atgttctttc ctgcgttatc ccctgattct gtggataacc gtattaccgc |
| 5881 | ctttgagtga gctgataccg ctcgccgcag ccgaacgacc gagcgcagcg agtcagtgag |
| 5941 | cgaggaagcg gaagagcgcc caatacgcaa accgcctctc cccgcgcgtt ggccgattca |
| 6001 | ttaatg |
| // | |
| 1361_pAAV.Blimp1.1.0 kb.MND.GFP.pA | |
| (SEQ ID NO: 54) | |
| LOCUS (#1361)\pAAV.Bli 7206 bp DNA circular UNA 10-MAR.-2017 | |
| DEFINITION GFP in AAV, via MD expression cassette, ie, CMV, globin intron, | |
| GFP, globin polyA. This plasmid sequence was corrected on | |
| Oct. 3, 2002, with correction record #201. AAV non-vector error, | |
| probably present in all AAV constructs. Merely deletes 3 bp prior | |
| to left ITR sequence. Replacement of CAGCAGCTGC GCGCTCGCTC GCTCA | |
| with CAGCTGCGCG CTCGCTCGCT CA. | |
| ACCESSION urn.local . . . g-6q4aps1 | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac acgcgtggta |
| 181 | aaccatgaac atcagaaaga cttttattaa cctatgacag ggtccccacc ccagtatttt |
| 241 | tccactccat taaaatggaa gttttttttt tttttttctt ttttgagaca gagttttgct |
| 301 | cttgttgccc agtctggagt gcaatggcac aatctcggct caccacaacc tccacctccc |
| 361 | agattcaagc gattcttctg cctcagcctc ccaagtagct gggattacag gtgtgcgcca |
| 421 | ccacgcccag ctaattttgt atttttagta gagatggggt ttctccatgt tggtcaggct |
| 481 | ggtctcgaac ttccgacctc aggtgatccg cccacctcgg cctcccaaag tgctgggatt |
| 541 | acaggcaaga gccactgcat ccagcttagg ctatcttact ccagcctaaa cagcaatttt |
| 601 | ctatcataag gtctgtacta atgaaaacag aatcacccaa ggctgctgtt tgttctgtct |
| 661 | gtgctgccat tgtccgcatt ttgctgagga ggaaacggaa ctgcactttt gagtgagtgg |
| 721 | cccagagcct tctagaatga gagtgcgttg gaagccagat atgtggcgat tgtgtcgcca |
| 781 | gctgttactc aggttttctc aagaaggagg agcaactttg gcagttttgc ttcagttctc |
| 841 | tctagccctc tgtgtaatcg cccctttttc tttatttcag cacaaacaca gagcagtcta |
| 901 | aagcaaccga gcactgagaa aaatgaactc tgcccaaaga atgtcccaaa gagagagtac |
| 961 | agcgtgaaag aaatcctaaa attggactcc aacccctcca aaggaaagga cctctaccgt |
| 1021 | tctaacattt cacccctcac atcagaaaag gacctcgatg actttagaag acgtgggagc |
| 1081 | cccgaaatgc ccttctaccc tcgggtcgtt taccccatcc gggcccctct gccagaagac |
| 1141 | tttttgaaag cttccctggc ctacgggatc gagagagaac agagaaacag gagaatatgg |
| 1201 | gccaaacagg atatctgtgg taagcagttc ctgccccggc tcagggccaa gaacagttgg |
| 1261 | aacagcagaa tatgggccaa acaggatatc tgtggtaagc agttcctgcc ccggctcagg |
| 1321 | gccaagaaca gatggtcccc agatgcggtc ccgccctcag cagtttctag agaaccatca |
| 1381 | gatgtttcca gggtgcccca aggacctgaa atgaccctgt gccttatttg aactaaccaa |
| 1441 | tcagttcgct tctcgcttct gttcgcgcgc ttctgctccc cgagctctat ataagcagag |
| 1501 | ctcgtttagt gaaccgtcag atcgcctgga gacgccatcc acgctgtttt gacttccata |
| 1561 | gaaggatctc gaggccacca tggtgagcaa gggcgaggag ctgttcaccg gggtggtgcc |
| 1621 | catcctggtc gagctggacg gcgacgtaaa cggccacaag ttcagcgtgt ccggcgaggg |
| 1681 | cgagggcgat gccacctacg gcaagctgac cctgaagttc atctgcacca ccggcaagct |
| 1741 | gcccgtgccc tggcccaccc tcgtgaccac cctgacctac ggcgtgcagt gcttcagccg |
| 1801 | ctaccccgac cacatgaagc agcacgactt cttcaagtcc gccatgcccg aaggctacgt |
| 1861 | ccaggagcgc accatcttct tcaaggacga cggcaactac aagacccgcg ccgaggtgaa |
| 1921 | gttcgagggc gacaccctgg tgaaccgcat cgagctgaag ggcatcgact tcaaggagga |
| 1981 | cggcaacatc ctggggcaca agctggagta caactacaac agccacaacg tctatatcat |
| 2041 | ggccgacaag cagaagaacg gcatcaaggt gaacttcaag atccgccaca acatcgagga |
| 2101 | cggcagcgtg cagctcgccg accactacca gcagaacacc cccatcggcg acggccccgt |
| 2161 | gctgctgccc gacaaccact acctgagcac ccagtccgcc ctgagcaaag accccaacga |
| 2221 | gaagcgcgat cacatggtcc tgctggagtt cgtgaccgcc gccgggatca ctctcggcat |
| 2281 | ggacgagctg tacaagtaaa ctagtgtcga ctgctttatt tgtgaaattt gtgatgctat |
| 2341 | tgctttattt gtaaccatta taagctgcaa taaacaagtt aacaacaaca attgcattca |
| 2401 | ttttatgttt caggttcagg gggaggtgtg ggaggttttt taaaacgtac atcactcgct |
| 2461 | cccccattcc atcctccacc actccaagcc cctctgcaag aagcagcccc gaccaaagcc |
| 2521 | tcaagagctc cagccctcac agcagccctg ggaatacggt gtcccctgtg ggccccggct |
| 2581 | ctcaagagca ccgggactcc tacgcttact tgaacgcgtc ctacggcacg gaaggtttgg |
| 2641 | gctcctaccc tggctacgca cccctgcccc acctcccgcc agctttcatc ccctcgtaca |
| 2701 | acgctcacta ccccaagttc ctcttgcccc cctacggcat gaattgtaat ggcctgagcg |
| 2761 | ctgtgagcag catgaatggc atcaacaact ttggcctctt cccgaggctg tgccctgtct |
| 2821 | acagcaatct cctcggtggg ggcagcctgc cccaccccat gctcaacccc acttctctcc |
| 2881 | cgagctcgct gccctcagat ggagcccgga ggttgctcca gccggagcat cccagggagg |
| 2941 | tgcttgtccc ggcgccccac agtgccttct cctttaccgg ggccgccgcc agcatgaagg |
| 3001 | acaaggcctg tagccccaca agcgggtctc ccacggcggg aacagccgcc acggcagaac |
| 3061 | atgtggtgca gcccaaagct acctcagcag cgatggcagc ccccagcagc gacgaagcca |
| 3121 | tgaatctcat taaaaacaaa agaaacatga ccggctacaa gacccttccc tacccgctga |
| 3181 | agaagcagaa cggcaagatc aagtacgaat gcaacgtttg cgccaagact ttcggccagc |
| 3241 | tctccaatct gaaggtaggc cttgagagag agcagtccaa ggggctgtga gtgcatgctt |
| 3301 | gtgtttgtat ttagcttgct ttccatgggg tatcgattgc atttgcagta gtatgagccc |
| 3361 | ccggttgggg atagtgggta tggattccgc ctggcttttg ccacttctag ctctttgact |
| 3421 | ttggacaagt gacttccctt ctcctctaga gtagataagt agcatggcgg gttaatcatt |
| 3481 | aactacaagg aacccctagt gatggagttg gccactccct ctctgcgcgc tcgctcgctc |
| 3541 | actgaggccg ggcgaccaaa ggtcgcccga cgcccgggct ttgcccgggc ggcctcagtg |
| 3601 | agcgagcgag cgcgccagct ggcgtaatag cgaagaggcc cgcaccgatc gcccttccca |
| 3661 | acagttgcgc agcctgaatg gcgaatggcg attccgttgc aatggctggc ggtaatattg |
| 3721 | ttctggatat taccagcaag gccgatagtt tgagttcttc tactcaggca agtgatgtta |
| 3781 | ttactaatca aagaagtatt gcgacaacgg ttaatttgcg tgatggacag actcttttac |
| 3841 | tcggtggcct cactgattat aaaaacactt ctcaggattc tggcgtaccg ttcctgtcta |
| 3901 | aaatcccttt aatcggcctc ctgtttagct cccgctctga ttctaacgag gaaagcacgt |
| 3961 | tatacgtgct cgtcaaagca accatagtac gcgccctgta gcggcgcatt aagcgcggcg |
| 4021 | ggtgtggtgg ttacgcgcag cgtgaccgct acacttgcca gcgccctagc gcccgctcct |
| 4081 | ttcgctttct tcccttcctt tctcgccacg ttcgccggct ttccccgtca agctctaaat |
| 4141 | cgggggctcc ctttagggtt ccgatttagt gctttacggc acctcgaccc caaaaaactt |
| 4201 | gattagggtg atggttcacg tagtgggcca tcgccctgat agacggtttt tcgccctttg |
| 4261 | acgttggagt ccacgttctt taatagtgga ctcttgttcc aaactggaac aacactcaac |
| 4321 | cctatctcgg tctattcttt tgatttataa gggattttgc cgatttcggc ctattggtta |
| 4381 | aaaaatgagc tgatttaaca aaaatttaac gcgaatttta acaaaatatt aacgtttaca |
| 4441 | atttaaatat ttgcttatac aatcttcctg tttttggggc ttttctgatt atcaaccggg |
| 4501 | gtacatatga ttgacatgct agttttacga ttaccgttca tcgattctct tgtttgctcc |
| 4561 | agactctcag gcaatgacct gatagccttt gtagagacct ctcaaaaata gctaccctct |
| 4621 | ccggcatgaa tttatcagct agaacggttg aatatcatat tgatggtgat ttgactgtct |
| 4681 | ccggcctttc tcacccgttt gaatctttac ctacacatta ctcaggcatt gcatttaaaa |
| 4741 | tatatgaggg ttctaaaaat ttttatcctt gcgttgaaat aaaggcttct cccgcaaaag |
| 4801 | tattacaggg tcataatgtt tttggtacaa ccgatttagc tttatgctct gaggctttat |
| 4861 | tgcttaattt tgctaattct ttgccttgcc tgtatgattt attggatgtt ggaatcgcct |
| 4921 | gatgcggtat tttctcctta cgcatctgtg cggtatttca caccgcatat ggtgcactct |
| 4981 | cagtacaatc tgctctgatg ccgcatagtt aagccagccc cgacacccgc caacacccgc |
| 5041 | tgacgcgccc tgacgggctt gtctgctccc ggcatccgct tacagacaag ctgtgaccgt |
| 5101 | ctccgggagc tgcatgtgtc agaggttttc accgtcatca ccgaaacgcg cgagacgaaa |
| 5161 | gggcctcgtg atacgcctat ttttataggt taatgtcatg ataataatgg tttcttagac |
| 5221 | gtcaggtggc acttttcggg gaaatgtgcg cggaacccct atttgtttat ttttctaaat |
| 5281 | acattcaaat atgtatccgc tcatgagaca ataaccctga taaatgcttc aataatattg |
| 5341 | aaaaaggaag agtatgagta ttcaacattt ccgtgtcgcc cttattccct tttttgcggc |
| 5401 | attttgcctt cctgtttttg ctcacccaga aacgctggtg aaagtaaaag atgctgaaga |
| 5461 | tcagttgggt gcacgagtgg gttacatcga actggatctc aacagcggta agatccttga |
| 5521 | gagttttcgc cccgaagaac gttttccaat gatgagcact tttaaagttc tgctatgtgg |
| 5581 | cgcggtatta tcccgtattg acgccgggca agagcaactc ggtcgccgca tacactattc |
| 5641 | tcagaatgac ttggttgagt actcaccagt cacagaaaag catcttacgg atggcatgac |
| 5701 | agtaagagaa ttatgcagtg ctgccataac catgagtgat aacactgcgg ccaacttact |
| 5761 | tctgacaacg atcggaggac cgaaggagct aaccgctttt ttgcacaaca tgggggatca |
| 5821 | tgtaactcgc cttgatcgtt gggaaccgga gctgaatgaa gccataccaa acgacgagcg |
| 5881 | tgacaccacg atgcctgtag caatggcaac aacgttgcgc aaactattaa ctggcgaact |
| 5941 | acttactcta gcttcccggc aacaattaat agactggatg gaggcggata aagttgcagg |
| 6001 | accacttctg cgctcggccc ttccggctgg ctggtttatt gctgataaat ctggagccgg |
| 6061 | tgagcgtggg tctcgcggta tcattgcagc actggggcca gatggtaagc cctcccgtat |
| 6121 | cgtagttatc tacacgacgg ggagtcaggc aactatggat gaacgaaata gacagatcgc |
| 6181 | tgagataggt gcctcactga ttaagcattg gtaactgtca gaccaagttt actcatatat |
| 6241 | actttagatt gatttaaaac ttcattttta atttaaaagg atctaggtga agatcctttt |
| 6301 | tgataatctc atgaccaaaa tcccttaacg tgagttttcg ttccactgag cgtcagaccc |
| 6361 | cgtagaaaag atcaaaggat cttcttgaga tccttttttt ctgcgcgtaa tctgctgctt |
| 6421 | gcaaacaaaa aaaccaccgc taccagcggt ggtttgtttg ccggatcaag agctaccaac |
| 6481 | tctttttccg aaggtaactg gcttcagcag agcgcagata ccaaatactg tccttctagt |
| 6541 | gtagccgtag ttaggccacc acttcaagaa ctctgtagca ccgcctacat acctcgctct |
| 6601 | gctaatcctg ttaccagtgg ctgctgccag tggcgataag tcgtgtctta ccgggttgga |
| 6661 | ctcaagacga tagttaccgg ataaggcgca gcggtcgggc tgaacggggg gttcgtgcac |
| 6721 | acagcccagc ttggagcgaa cgacctacac cgaactgaga tacctacagc gtgagctatg |
| 6781 | agaaagcgcc acgcttcccg aagggagaaa ggcggacagg tatccggtaa gcggcagggt |
| 6841 | cggaacagga gagcgcacga gggagcttcc agggggaaac gcctggtatc tttatagtcc |
| 6901 | tgtcgggttt cgccacctct gacttgagcg tcgatttttg tgatgctcgt caggggggcg |
| 6961 | gagcctatgg aaaaacgcca gcaacgcggc ctttttacgg ttcctggcct tttgctggcc |
| 7021 | ttttgctcac atgttctttc ctgcgttatc ccctgattct gtggataacc gtattaccgc |
| 7081 | ctttgagtga gctgataccg ctcgccgcag ccgaacgacc gagcgcagcg agtcagtgag |
| 7141 | cgaggaagcg gaagagcgcc caatacgcaa accgcctctc cccgcgcgtt ggccgattca |
| 7201 | ttaatg |
| // | |
| 1366_CCR5.MND.BAFF_CRISPR.HR | |
| (SEQ ID NO: 55) | |
| LOCUS #1366\CCR5.MND.B 6707 bp DNA circular SYN 10-MAR.-2017 | |
| . | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga ggccaccatg taccggatgc agctgctgag ctgcatcgca |
| 1501 | ctgagcctgg cactggtgac caacagcgca gtgcagggac cagaggagac cgtgacccag |
| 1561 | gactgcctgc agctgatcgc agacagcgag acccccacca tccagaaggg cagctacacc |
| 1621 | ttcgtgccct ggctgctgag cttcaagcgg ggcagcgccc tggaggagaa ggagaacaag |
| 1681 | attctggtga aggagaccgg ctacttcttc atctacggcc aggtgctgta caccgataag |
| 1741 | acctacgcca tgggccacct gatccagcgg aagaaggtgc acgtgttcgg cgacgagctg |
| 1801 | agcctggtga ccctgttccg gtgcatccag aacatgcccg agaccctgcc caacaacagc |
| 1861 | tgctacagcg caggaatcgc aaagctggag gagggcgacg agctgcagct ggcaatcccc |
| 1921 | cgggagaacg cacagatcag cctggacggc gacgtgacct tcttcggcgc cctgaagctc |
| 1981 | ctgtgagtcg actgctttat ttgtgaaatt tgtgatgcta ttgctttatt tgtaaccatt |
| 2041 | ataagctgca ataaacaagt taacaacaac aattgcattc attttatgtt tcaggttcag |
| 2101 | ggggaggtgt gggaggtttt ttaaactcta ttttataggc ttcttctctg gaatcttctt |
| 2161 | catcatcctc ctgacaatcg ataggtacct ggctgtcgtc catgctgtgt ttgctttaaa |
| 2221 | agccaggacg gtcacctttg gggtggtgac aagtgtgatc acttgggtgg tggctgtgtt |
| 2281 | tgcgtctctc ccaggaatca tctttaccag atctcaaaaa gaaggtcttc attacacctg |
| 2341 | cagctctcat tttccataca gtcagtatca attctggaag aatttccaga cattaaagat |
| 2401 | agtcatcttg gggctggtcc tgccgctgct tgtcatggtc atctgctact cgggaatcct |
| 2461 | aaaaactctg cttcggtgtc gaaatgagaa gaagaggcac agggctgtga ggcttatctt |
| 2521 | caccatcatg attgtttatt ttctcttctg ggctccctac aacattgtcc ttctcctgaa |
| 2581 | caccttccag gaattctttg gcctgaataa ttgcagtagc tctaacaggt tggaccaagc |
| 2641 | tatgcaggtg acagagactc ttgggatgac gcactgctgc atcaacccca tcatctatgc |
| 2701 | ctttgtcggg gagaagttca gaaactacct cttagtcttc ttccaaaagc acattgccaa |
| 2761 | acgcttctgc aaatgctgtt ctattttcca gcaagaggct cccgagcgag caagctcagt |
| 2821 | ttacacccga tccactgggg agcaggaaat atctgtgggc ttgtgacacg gactcaagtg |
| 2881 | ggctggtgac ccagtcagag ttgtgcacat ggcttagttt tcatacacac cgcggtctag |
| 2941 | agcatggcta cgtagataag tagcatggcg ggttaatcat taactacaag gaacccctag |
| 3001 | tgatggagtt ggccactccc tctctgcgcg ctcgctcgct cactgaggcc gggcgaccaa |
| 3061 | aggtcgcccg acgcccgggc tttgcccggg cggcctcagt gagcgagcga gcgcgccagc |
| 3121 | tggcgtaata gcgaagaggc ccgcaccgat cgcccttccc aacagttgcg cagcctgaat |
| 3181 | ggcgaatggc gattccgttg caatggctgg cggtaatatt gttctggata ttaccagcaa |
| 3241 | ggccgatagt ttgagttctt ctactcaggc aagtgatgtt attactaatc aaagaagtat |
| 3301 | tgcgacaacg gttaatttgc gtgatggaca gactctttta ctcggtggcc tcactgatta |
| 3361 | taaaaacact tctcaggatt ctggcgtacc gttcctgtct aaaatccctt taatcggcct |
| 3421 | cctgtttagc tcccgctctg attctaacga ggaaagcacg ttatacgtgc tcgtcaaagc |
| 3481 | aaccatagta cgcgccctgt agcggcgcat taagcgcggc gggtgtggtg gttacgcgca |
| 3541 | gcgtgaccgc tacacttgcc agcgccctag cgcccgctcc tttcgctttc ttcccttcct |
| 3601 | ttctcgccac gttcgccggc tttccccgtc aagctctaaa tcgggggctc cctttagggt |
| 3661 | tccgatttag tgctttacgg cacctcgacc ccaaaaaact tgattagggt gatggttcac |
| 3721 | gtagtgggcc atcgccctga tagacggttt ttcgcccttt gacgttggag tccacgttct |
| 3781 | ttaatagtgg actcttgttc caaactggaa caacactcaa ccctatctcg gtctattctt |
| 3841 | ttgatttata agggattttg ccgatttcgg cctattggtt aaaaaatgag ctgatttaac |
| 3901 | aaaaatttaa cgcgaatttt aacaaaatat taacgtttac aatttaaata tttgcttata |
| 3961 | caatcttcct gtttttgggg cttttctgat tatcaaccgg ggtacatatg attgacatgc |
| 4021 | tagttttacg attaccgttc atcgattctc ttgtttgctc cagactctca ggcaatgacc |
| 4081 | tgatagcctt tgtagagacc tctcaaaaat agctaccctc tccggcatga atttatcagc |
| 4141 | tagaacggtt gaatatcata ttgatggtga tttgactgtc tccggccttt ctcacccgtt |
| 4201 | tgaatcttta cctacacatt actcaggcat tgcatttaaa atatatgagg gttctaaaaa |
| 4261 | tttttatcct tgcgttgaaa taaaggcttc tcccgcaaaa gtattacagg gtcataatgt |
| 4321 | ttttggtaca accgatttag ctttatgctc tgaggcttta ttgcttaatt ttgctaattc |
| 4381 | tttgccttgc ctgtatgatt tattggatgt tggaatcgcc tgatgcggta ttttctcctt |
| 4441 | acgcatctgt gcggtatttc acaccgcata tggtgcactc tcagtacaat ctgctctgat |
| 4501 | gccgcatagt taagccagcc ccgacacccg ccaacacccg ctgacgcgcc ctgacgggct |
| 4561 | tgtctgctcc cggcatccgc ttacagacaa gctgtgaccg tctccgggag ctgcatgtgt |
| 4621 | cagaggtttt caccgtcatc accgaaacgc gcgagacgaa agggcctcgt gatacgccta |
| 4681 | tttttatagg ttaatgtcat gataataatg gtttcttaga cgtcaggtgg cacttttcgg |
| 4741 | ggaaatgtgc gcggaacccc tatttgttta tttttctaaa tacattcaaa tatgtatccg |
| 4801 | ctcatgagac aataaccctg ataaatgctt caataatatt gaaaaaggaa gagtatgagt |
| 4861 | attcaacatt tccgtgtcgc ccttattccc ttttttgcgg cattttgcct tcctgttttt |
| 4921 | gctcacccag aaacgctggt gaaagtaaaa gatgctgaag atcagttggg tgcacgagtg |
| 4981 | ggttacatcg aactggatct caacagcggt aagatccttg agagttttcg ccccgaagaa |
| 5041 | cgttttccaa tgatgagcac ttttaaagtt ctgctatgtg gcgcggtatt atcccgtatt |
| 5101 | gacgccgggc aagagcaact cggtcgccgc atacactatt ctcagaatga cttggttgag |
| 5161 | tactcaccag tcacagaaaa gcatcttacg gatggcatga cagtaagaga attatgcagt |
| 5221 | gctgccataa ccatgagtga taacactgcg gccaacttac ttctgacaac gatcggagga |
| 5281 | ccgaaggagc taaccgcttt tttgcacaac atgggggatc atgtaactcg ccttgatcgt |
| 5341 | tgggaaccgg agctgaatga agccatacca aacgacgagc gtgacaccac gatgcctgta |
| 5401 | gcaatggcaa caacgttgcg caaactatta actggcgaac tacttactct agcttcccgg |
| 5461 | caacaattaa tagactggat ggaggcggat aaagttgcag gaccacttct gcgctcggcc |
| 5521 | cttccggctg gctggtttat tgctgataaa tctggagccg gtgagcgtgg gtctcgcggt |
| 5581 | atcattgcag cactggggcc agatggtaag ccctcccgta tcgtagttat ctacacgacg |
| 5641 | gggagtcagg caactatgga tgaacgaaat agacagatcg ctgagatagg tgcctcactg |
| 5701 | attaagcatt ggtaactgtc agaccaagtt tactcatata tactttagat tgatttaaaa |
| 5761 | cttcattttt aatttaaaag gatctaggtg aagatccttt ttgataatct catgaccaaa |
| 5821 | atcccttaac gtgagttttc gttccactga gcgtcagacc ccgtagaaaa gatcaaagga |
| 5881 | tcttcttgag atcctttttt tctgcgcgta atctgctgct tgcaaacaaa aaaaccaccg |
| 5941 | ctaccagcgg tggtttgttt gccggatcaa gagctaccaa ctctttttcc gaaggtaact |
| 6001 | ggcttcagca gagcgcagat accaaatact gtccttctag tgtagccgta gttaggccac |
| 6061 | cacttcaaga actctgtagc accgcctaca tacctcgctc tgctaatcct gttaccagtg |
| 6121 | gctgctgcca gtggcgataa gtcgtgtctt accgggttgg actcaagacg atagttaccg |
| 6181 | gataaggcgc agcggtcggg ctgaacgggg ggttcgtgca cacagcccag cttggagcga |
| 6241 | acgacctaca ccgaactgag atacctacag cgtgagctat gagaaagcgc cacgcttccc |
| 6301 | gaagggagaa aggcggacag gtatccggta agcggcaggg tcggaacagg agagcgcacg |
| 6361 | agggagcttc cagggggaaa cgcctggtat ctttatagtc ctgtcgggtt tcgccacctc |
| 6421 | tgacttgagc gtcgattttt gtgatgctcg tcaggggggc ggagcctatg gaaaaacgcc |
| 6481 | agcaacgcgg cctttttacg gttcctggcc ttttgctggc cttttgctca catgttcttt |
| 6541 | cctgcgttat cccctgattc tgtggataac cgtattaccg cctttgagtg agctgatacc |
| 6601 | gctcgccgca gccgaacgac cgagcgcagc gagtcagtga gcgaggaagc ggaagagcgc |
| 6661 | ccaatacgca aaccgcctct ccccgcgcgt tggccgattc attaatg |
| // | |
| 1367_CCR5.MND.BAFF.2A.GFP | |
| (SEQ ID NO: 56) | |
| LOCUS #1367\CCR5.MND.B 7494 bp DNA circular SYN 10-MAR.-2017 | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga ggccaccatg taccggatgc agctgctgag ctgcatcgca |
| 1501 | ctgagcctgg cactggtgac caacagcgca gtgcagggac cagaggagac cgtgacccag |
| 1561 | gactgcctgc agctgatcgc agacagcgag acccccacca tccagaaggg cagctacacc |
| 1621 | ttcgtgccct ggctgctgag cttcaagcgg ggcagcgccc tggaggagaa ggagaacaag |
| 1681 | attctggtga aggagaccgg ctacttcttc atctacggcc aggtgctgta caccgataag |
| 1741 | acctacgcca tgggccacct gatccagcgg aagaaggtgc acgtgttcgg cgacgagctg |
| 1801 | agcctggtga ccctgttccg gtgcatccag aacatgcccg agaccctgcc caacaacagc |
| 1861 | tgctacagcg caggaatcgc aaagctggag gagggcgacg agctgcagct ggcaatcccc |
| 1921 | cgggagaacg cacagatcag cctggacggc gacgtgacct tcttcggcgc cctgaagctc |
| 1981 | ctgggatccg gtgagggcag aggaagtctt ctaacatgcg gtgacgtgga ggagaatccg |
| 2041 | ggccccatgg tgagcaaggg cgaggagctg ttcaccgggg tggtgcccat cctggtcgag |
| 2101 | ctggacggcg acgtaaacgg ccacaagttc agcgtgtccg gcgagggcga gggcgatgcc |
| 2161 | acctacggca agctgaccct gaagttcatc tgcaccaccg gcaagctgcc cgtgccctgg |
| 2221 | cccaccctcg tgaccaccct gacctacggc gtgcagtgct tcagccgcta ccccgaccac |
| 2281 | atgaagcagc acgacttctt caagtccgcc atgcccgaag gctacgtcca ggagcgcacc |
| 2341 | atcttcttca aggacgacgg caactacaag acccgcgccg aggtgaagtt cgagggcgac |
| 2401 | accctggtga accgcatcga gctgaagggc atcgacttca aggaggacgg caacatcctg |
| 2461 | gggcacaagc tggagtacaa ctacaacagc cacaacgtct atatcatggc cgacaagcag |
| 2521 | aagaacggca tcaaggtgaa cttcaagatc cgccacaaca tcgaggacgg cagcgtgcag |
| 2581 | ctcgccgacc actaccagca gaacaccccc atcggcgacg gccccgtgct gctgcccgac |
| 2641 | aaccactacc tgagcaccca gtccgccctg agcaaagacc ccaacgagaa gcgcgatcac |
| 2701 | atggtcctgc tggagttcgt gaccgccgcc gggatcactc tcggcatgga cgagctgtac |
| 2761 | aagtgaatct agagtcgact gctttatttg tgaaatttgt gatgctattg ctttatttgt |
| 2821 | aaccattata agctgcaata aacaagttaa caacaacaat tgcattcatt ttatgtttca |
| 2881 | ggttcagggg gaggtgtggg aggtttttta aactctattt tataggcttc ttctctggaa |
| 2941 | tcttcttcat catcctcctg acaatcgata ggtacctggc tgtcgtccat gctgtgtttg |
| 3001 | ctttaaaagc caggacggtc acctttgggg tggtgacaag tgtgatcact tgggtggtgg |
| 3061 | ctgtgtttgc gtctctccca ggaatcatct ttaccagatc tcaaaaagaa ggtcttcatt |
| 3121 | acacctgcag ctctcatttt ccatacagtc agtatcaatt ctggaagaat ttccagacat |
| 3181 | taaagatagt catcttgggg ctggtcctgc cgctgcttgt catggtcatc tgctactcgg |
| 3241 | gaatcctaaa aactctgctt cggtgtcgaa atgagaagaa gaggcacagg gctgtgaggc |
| 3301 | ttatcttcac catcatgatt gtttattttc tcttctgggc tccctacaac attgtccttc |
| 3361 | tcctgaacac cttccaggaa ttctttggcc tgaataattg cagtagctct aacaggttgg |
| 3421 | accaagctat gcaggtgaca gagactcttg ggatgacgca ctgctgcatc aaccccatca |
| 3481 | tctatgcctt tgtcggggag aagttcagaa actacctctt agtcttcttc caaaagcaca |
| 3541 | ttgccaaacg cttctgcaaa tgctgttcta ttttccagca agaggctccc gagcgagcaa |
| 3601 | gctcagttta cacccgatcc actggggagc aggaaatatc tgtgggcttg tgacacggac |
| 3661 | tcaagtgggc tggtgaccca gtcagagttg tgcacatggc ttagttttca tacacaccgc |
| 3721 | ggtctagagc atggctacgt agataagtag catggcgggt taatcattaa ctacaaggaa |
| 3781 | cccctagtga tggagttggc cactccctct ctgcgcgctc gctcgctcac tgaggccggg |
| 3841 | cgaccaaagg tcgcccgacg cccgggcttt gcccgggcgg cctcagtgag cgagcgagcg |
| 3901 | cgccagctgg cgtaatagcg aagaggcccg caccgatcgc ccttcccaac agttgcgcag |
| 3961 | cctgaatggc gaatggcgat tccgttgcaa tggctggcgg taatattgtt ctggatatta |
| 4021 | ccagcaaggc cgatagtttg agttcttcta ctcaggcaag tgatgttatt actaatcaaa |
| 4081 | gaagtattgc gacaacggtt aatttgcgtg atggacagac tcttttactc ggtggcctca |
| 4141 | ctgattataa aaacacttct caggattctg gcgtaccgtt cctgtctaaa atccctttaa |
| 4201 | tcggcctcct gtttagctcc cgctctgatt ctaacgagga aagcacgtta tacgtgctcg |
| 4261 | tcaaagcaac catagtacgc gccctgtagc ggcgcattaa gcgcggcggg tgtggtggtt |
| 4321 | acgcgcagcg tgaccgctac acttgccagc gccctagcgc ccgctccttt cgctttcttc |
| 4381 | ccttcctttc tcgccacgtt cgccggcttt ccccgtcaag ctctaaatcg ggggctccct |
| 4441 | ttagggttcc gatttagtgc tttacggcac ctcgacccca aaaaacttga ttagggtgat |
| 4501 | ggttcacgta gtgggccatc gccctgatag acggtttttc gccctttgac gttggagtcc |
| 4561 | acgttcttta atagtggact cttgttccaa actggaacaa cactcaaccc tatctcggtc |
| 4621 | tattcttttg atttataagg gattttgccg atttcggcct attggttaaa aaatgagctg |
| 4681 | atttaacaaa aatttaacgc gaattttaac aaaatattaa cgtttacaat ttaaatattt |
| 4741 | gcttatacaa tcttcctgtt tttggggctt ttctgattat caaccggggt acatatgatt |
| 4801 | gacatgctag ttttacgatt accgttcatc gattctcttg tttgctccag actctcaggc |
| 4861 | aatgacctga tagcctttgt agagacctct caaaaatagc taccctctcc ggcatgaatt |
| 4921 | tatcagctag aacggttgaa tatcatattg atggtgattt gactgtctcc ggcctttctc |
| 4981 | acccgtttga atctttacct acacattact caggcattgc atttaaaata tatgagggtt |
| 5041 | ctaaaaattt ttatccttgc gttgaaataa aggcttctcc cgcaaaagta ttacagggtc |
| 5101 | ataatgtttt tggtacaacc gatttagctt tatgctctga ggctttattg cttaattttg |
| 5161 | ctaattcttt gccttgcctg tatgatttat tggatgttgg aatcgcctga tgcggtattt |
| 5221 | tctccttacg catctgtgcg gtatttcaca ccgcatatgg tgcactctca gtacaatctg |
| 5281 | ctctgatgcc gcatagttaa gccagccccg acacccgcca acacccgctg acgcgccctg |
| 5341 | acgggcttgt ctgctcccgg catccgctta cagacaagct gtgaccgtct ccgggagctg |
| 5401 | catgtgtcag aggttttcac cgtcatcacc gaaacgcgcg agacgaaagg gcctcgtgat |
| 5461 | acgcctattt ttataggtta atgtcatgat aataatggtt tcttagacgt caggtggcac |
| 5521 | ttttcgggga aatgtgcgcg gaacccctat ttgtttattt ttctaaatac attcaaatat |
| 5581 | gtatccgctc atgagacaat aaccctgata aatgcttcaa taatattgaa aaaggaagag |
| 5641 | tatgagtatt caacatttcc gtgtcgccct tattcccttt tttgcggcat tttgccttcc |
| 5701 | tgtttttgct cacccagaaa cgctggtgaa agtaaaagat gctgaagatc agttgggtgc |
| 5761 | acgagtgggt tacatcgaac tggatctcaa cagcggtaag atccttgaga gttttcgccc |
| 5821 | cgaagaacgt tttccaatga tgagcacttt taaagttctg ctatgtggcg cggtattatc |
| 5881 | ccgtattgac gccgggcaag agcaactcgg tcgccgcata cactattctc agaatgactt |
| 5941 | ggttgagtac tcaccagtca cagaaaagca tcttacggat ggcatgacag taagagaatt |
| 6001 | atgcagtgct gccataacca tgagtgataa cactgcggcc aacttacttc tgacaacgat |
| 6061 | cggaggaccg aaggagctaa ccgctttttt gcacaacatg ggggatcatg taactcgcct |
| 6121 | tgatcgttgg gaaccggagc tgaatgaagc cataccaaac gacgagcgtg acaccacgat |
| 6181 | gcctgtagca atggcaacaa cgttgcgcaa actattaact ggcgaactac ttactctagc |
| 6241 | ttcccggcaa caattaatag actggatgga ggcggataaa gttgcaggac cacttctgcg |
| 6301 | ctcggccctt ccggctggct ggtttattgc tgataaatct ggagccggtg agcgtgggtc |
| 6361 | tcgcggtatc attgcagcac tggggccaga tggtaagccc tcccgtatcg tagttatcta |
| 6421 | cacgacgggg agtcaggcaa ctatggatga acgaaataga cagatcgctg agataggtgc |
| 6481 | ctcactgatt aagcattggt aactgtcaga ccaagtttac tcatatatac tttagattga |
| 6541 | tttaaaactt catttttaat ttaaaaggat ctaggtgaag atcctttttg ataatctcat |
| 6601 | gaccaaaatc ccttaacgtg agttttcgtt ccactgagcg tcagaccccg tagaaaagat |
| 6661 | caaaggatct tcttgagatc ctttttttct gcgcgtaatc tgctgcttgc aaacaaaaaa |
| 6721 | accaccgcta ccagcggtgg tttgtttgcc ggatcaagag ctaccaactc tttttccgaa |
| 6781 | ggtaactggc ttcagcagag cgcagatacc aaatactgtc cttctagtgt agccgtagtt |
| 6841 | aggccaccac ttcaagaact ctgtagcacc gcctacatac ctcgctctgc taatcctgtt |
| 6901 | accagtggct gctgccagtg gcgataagtc gtgtcttacc gggttggact caagacgata |
| 6961 | gttaccggat aaggcgcagc ggtcgggctg aacggggggt tcgtgcacac agcccagctt |
| 7021 | ggagcgaacg acctacaccg aactgagata cctacagcgt gagctatgag aaagcgccac |
| 7081 | gcttcccgaa gggagaaagg cggacaggta tccggtaagc ggcagggtcg gaacaggaga |
| 7141 | gcgcacgagg gagcttccag ggggaaacgc ctggtatctt tatagtcctg tcgggtttcg |
| 7201 | ccacctctga cttgagcgtc gatttttgtg atgctcgtca ggggggcgga gcctatggaa |
| 7261 | aaacgccagc aacgcggcct ttttacggtt cctggccttt tgctggcctt ttgctcacat |
| 7321 | gttctttcct gcgttatccc ctgattctgt ggataaccgt attaccgcct ttgagtgagc |
| 7381 | tgataccgct cgccgcagcc gaacgaccga gcgcagcgag tcagtgagcg aggaagcgga |
| 7441 | agagcgccca atacgcaaac cgcctctccc cgcgcgttgg ccgattcatt aatg |
| // | |
| 1378_CCR5.MND.mCherry.2A.coFIXpadua.WPRE.pa | |
| (SEQ ID NO: 57) | |
| LOCUS #1378 8330 bp DNA circular UNA 09-AUG.-2017 | |
| DEFINITION Gibson Assembly of mCherry T2A - coFIXpadua into 1367 XhoI + SalI | |
| (6.2 kb). | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga ggccaccatg gtgagcaagg gcgaggagga taacatggcc |
| 1501 | atcatcaagg agttcatgcg cttcaaggtg cacatggagg gctccgtgaa cggccacgag |
| 1561 | ttcgagatcg agggcgaggg cgagggccgc ccctacgagg gcacccagac cgccaagctg |
| 1621 | aaggtgacca agggtggccc cctgcccttc gcctgggaca tcctgtcccc tcagttcatg |
| 1681 | tacggctcca aggcctacgt gaagcacccc gccgacatcc ccgactactt gaagctgtcc |
| 1741 | ttccccgagg gcttcaagtg ggagcgcgtg atgaacttcg aggacggcgg cgtggtgacc |
| 1801 | gtgacccagg actcctctct gcaggacggc gagttcatct acaaggtgaa gctgcgcggc |
| 1861 | accaacttcc cctccgacgg ccccgtaatg cagaagaaga ccatgggctg ggaggcctcc |
| 1921 | tccgagcgga tgtaccccga ggacggcgcc ctgaagggcg agatcaagca gaggctgaag |
| 1981 | ctgaaggacg gcggccacta cgacgctgag gtcaagacca cctacaaggc caagaagccc |
| 2041 | gtgcagctgc ccggcgccta caacgtcaac atcaagttgg acatcacctc ccacaacgag |
| 2101 | gactacacca tcgtggaaca gtacgaacgc gccgagggcc gccactccac cggcggcatg |
| 2161 | gacgagctgt acaagggatc cggtgagggc agaggaagtc ttctaacatg cggtgacgtg |
| 2221 | gaggagaatc cgggccccat gatcatggcc gagagccctg gcctgatcac catctgcctg |
| 2281 | ctgggctacc tgctgagcgc cgagtgcacc gtgttcctgg accacgagaa cgccaacaag |
| 2341 | atcctgaacc ggcccaagag atacaacagc ggcaagctgg aggagttcgt gcagggcaac |
| 2401 | ctggagaggg agtgcatgga ggagaagtgc agcttcgagg aggccaggga agtgttcgag |
| 2461 | aacaccgagc ggaccaccga gttctggaag cagtacgtgg acggcgacca gtgcgagagc |
| 2521 | aacccttgcc tgaacggcgg cagctgcaag gacgacatca acagctacga gtgctggtgc |
| 2581 | cctttcggct tcgagggcaa gaactgcgag ctggacgtga cctgcaacat caagaacggc |
| 2641 | cgctgcgagc agttctgcaa gaacagcgcc gacaacaaag tggtgtgtag ctgcaccgag |
| 2701 | ggctacagac tggccgagaa ccagaagagc tgcgagcccg ccgtgccctt cccctgcggc |
| 2761 | agagtgagcg tgtcccagac cagcaagctg accagagccg agaccgtgtt ccccgacgtg |
| 2821 | gactacgtga atagcaccga ggccgagacc atcctggaca acatcaccca gagcacccag |
| 2881 | tccttcaacg acttcaccag agttgtgggc ggcgaggacg ccaagcccgg ccagttcccc |
| 2941 | tggcaggtgg tgctgaacgg caaagtggat gccttctgcg gcggcagcat cgtgaacgag |
| 3001 | aagtggatcg tgacagccgc ccactgcgtg gagaccggcg tgaagatcac cgtggtggcc |
| 3061 | ggcgaacaca atatcgagga gaccgagcac accgagcaga agcggaacgt catccggatt |
| 3121 | atcccccacc acaactacaa cgccgccatc aacaagtaca accacgacat cgccctgctg |
| 3181 | gagctggacg agcctctggt gctgaatagc tacgtgaccc ccatctgcat cgccgacaag |
| 3241 | gagtacacca acatcttcct gaagttcggc agcggctacg tgtccggctg gggcagagtg |
| 3301 | ttccacaagg gcagaagcgc cctggtgctg cagtacctga gagtgcccct ggtggacaga |
| 3361 | gccacctgcc tgttgagcac caagttcacc atctacaaca acatgttctg cgccggcttc |
| 3421 | cacgagggcg gcagagacag ctgccagggc gacagcggcg gaccccacgt gaccgaagtg |
| 3481 | gagggcacca gcttcctgac cggcatcatc agctggggcg aggagtgcgc catgaagggc |
| 3541 | aagtacggca tctacaccaa agtgagccgg tacgtgaact ggatcaagga gaaaaccaag |
| 3601 | ctgacctgag tcgactgctt tatttgtgaa atttgtgatg ctattgcttt atttgtaacc |
| 3661 | attataagct gcaataaaca agttaacaac aacaattgca ttcattttat gtttcaggtt |
| 3721 | cagggggagg tgtgggaggt tttttaaact ctattttata ggcttcttct ctggaatctt |
| 3781 | cttcatcatc ctcctgacaa tcgataggta cctggctgtc gtccatgctg tgtttgcttt |
| 3841 | aaaagccagg acggtcacct ttggggtggt gacaagtgtg atcacttggg tggtggctgt |
| 3901 | gtttgcgtct ctcccaggaa tcatctttac cagatctcaa aaagaaggtc ttcattacac |
| 3961 | ctgcagctct cattttccat acagtcagta tcaattctgg aagaatttcc agacattaaa |
| 4021 | gatagtcatc ttggggctgg tcctgccgct gcttgtcatg gtcatctgct actcgggaat |
| 4081 | cctaaaaact ctgcttcggt gtcgaaatga gaagaagagg cacagggctg tgaggcttat |
| 4141 | cttcaccatc atgattgttt attttctctt ctgggctccc tacaacattg tccttctcct |
| 4201 | gaacaccttc caggaattct ttggcctgaa taattgcagt agctctaaca ggttggacca |
| 4261 | agctatgcag gtgacagaga ctcttgggat gacgcactgc tgcatcaacc ccatcatcta |
| 4321 | tgcctttgtc ggggagaagt tcagaaacta cctcttagtc ttcttccaaa agcacattgc |
| 4381 | caaacgcttc tgcaaatgct gttctatttt ccagcaagag gctcccgagc gagcaagctc |
| 4441 | agtttacacc cgatccactg gggagcagga aatatctgtg ggcttgtgac acggactcaa |
| 4501 | gtgggctggt gacccagtca gagttgtgca catggcttag ttttcataca caccgcggtc |
| 4561 | tagagcatgg ctacgtagat aagtagcatg gcgggttaat cattaactac aaggaacccc |
| 4621 | tagtgatgga gttggccact ccctctctgc gcgctcgctc gctcactgag gccgggcgac |
| 4681 | caaaggtcgc ccgacgcccg ggctttgccc gggcggcctc agtgagcgag cgagcgcgcc |
| 4741 | agctggcgta atagcgaaga ggcccgcacc gatcgccctt cccaacagtt gcgcagcctg |
| 4801 | aatggcgaat ggcgattccg ttgcaatggc tggcggtaat attgttctgg atattaccag |
| 4861 | caaggccgat agtttgagtt cttctactca ggcaagtgat gttattacta atcaaagaag |
| 4921 | tattgcgaca acggttaatt tgcgtgatgg acagactctt ttactcggtg gcctcactga |
| 4981 | ttataaaaac acttctcagg attctggcgt accgttcctg tctaaaatcc ctttaatcgg |
| 5041 | cctcctgttt agctcccgct ctgattctaa cgaggaaagc acgttatacg tgctcgtcaa |
| 5101 | agcaaccata gtacgcgccc tgtagcggcg cattaagcgc ggcgggtgtg gtggttacgc |
| 5161 | gcagcgtgac cgctacactt gccagcgccc tagcgcccgc tcctttcgct ttcttccctt |
| 5221 | cctttctcgc cacgttcgcc ggctttcccc gtcaagctct aaatcggggg ctccctttag |
| 5281 | ggttccgatt tagtgcttta cggcacctcg accccaaaaa acttgattag ggtgatggtt |
| 5341 | cacgtagtgg gccatcgccc tgatagacgg tttttcgccc tttgacgttg gagtccacgt |
| 5401 | tctttaatag tggactcttg ttccaaactg gaacaacact caaccctatc tcggtctatt |
| 5461 | cttttgattt ataagggatt ttgccgattt cggcctattg gttaaaaaat gagctgattt |
| 5521 | aacaaaaatt taacgcgaat tttaacaaaa tattaacgtt tacaatttaa atatttgctt |
| 5581 | atacaatctt cctgtttttg gggcttttct gattatcaac cggggtacat atgattgaca |
| 5641 | tgctagtttt acgattaccg ttcatcgatt ctcttgtttg ctccagactc tcaggcaatg |
| 5701 | acctgatagc ctttgtagag acctctcaaa aatagctacc ctctccggca tgaatttatc |
| 5761 | agctagaacg gttgaatatc atattgatgg tgatttgact gtctccggcc tttctcaccc |
| 5821 | gtttgaatct ttacctacac attactcagg cattgcattt aaaatatatg agggttctaa |
| 5881 | aaatttttat ccttgcgttg aaataaaggc ttctcccgca aaagtattac agggtcataa |
| 5941 | tgtttttggt acaaccgatt tagctttatg ctctgaggct ttattgctta attttgctaa |
| 6001 | ttctttgcct tgcctgtatg atttattgga tgttggaatc gcctgatgcg gtattttctc |
| 6061 | cttacgcatc tgtgcggtat ttcacaccgc atatggtgca ctctcagtac aatctgctct |
| 6121 | gatgccgcat agttaagcca gccccgacac ccgccaacac ccgctgacgc gccctgacgg |
| 6181 | gcttgtctgc tcccggcatc cgcttacaga caagctgtga ccgtctccgg gagctgcatg |
| 6241 | tgtcagaggt tttcaccgtc atcaccgaaa cgcgcgagac gaaagggcct cgtgatacgc |
| 6301 | ctatttttat aggttaatgt catgataata atggtttctt agacgtcagg tggcactttt |
| 6361 | cggggaaatg tgcgcggaac ccctatttgt ttatttttct aaatacattc aaatatgtat |
| 6421 | ccgctcatga gacaataacc ctgataaatg cttcaataat attgaaaaag gaagagtatg |
| 6481 | agtattcaac atttccgtgt cgcccttatt cccttttttg cggcattttg ccttcctgtt |
| 6541 | tttgctcacc cagaaacgct ggtgaaagta aaagatgctg aagatcagtt gggtgcacga |
| 6601 | gtgggttaca tcgaactgga tctcaacagc ggtaagatcc ttgagagttt tcgccccgaa |
| 6661 | gaacgttttc caatgatgag cacttttaaa gttctgctat gtggcgcggt attatcccgt |
| 6721 | attgacgccg ggcaagagca actcggtcgc cgcatacact attctcagaa tgacttggtt |
| 6781 | gagtactcac cagtcacaga aaagcatctt acggatggca tgacagtaag agaattatgc |
| 6841 | agtgctgcca taaccatgag tgataacact gcggccaact tacttctgac aacgatcgga |
| 6901 | ggaccgaagg agctaaccgc ttttttgcac aacatggggg atcatgtaac tcgccttgat |
| 6961 | cgttgggaac cggagctgaa tgaagccata ccaaacgacg agcgtgacac cacgatgcct |
| 7021 | gtagcaatgg caacaacgtt gcgcaaacta ttaactggcg aactacttac tctagcttcc |
| 7081 | cggcaacaat taatagactg gatggaggcg gataaagttg caggaccact tctgcgctcg |
| 7141 | gcccttccgg ctggctggtt tattgctgat aaatctggag ccggtgagcg tgggtctcgc |
| 7201 | ggtatcattg cagcactggg gccagatggt aagccctccc gtatcgtagt tatctacacg |
| 7261 | acggggagtc aggcaactat ggatgaacga aatagacaga tcgctgagat aggtgcctca |
| 7321 | ctgattaagc attggtaact gtcagaccaa gtttactcat atatacttta gattgattta |
| 7381 | aaacttcatt tttaatttaa aaggatctag gtgaagatcc tttttgataa tctcatgacc |
| 7441 | aaaatccctt aacgtgagtt ttcgttccac tgagcgtcag accccgtaga aaagatcaaa |
| 7501 | ggatcttctt gagatccttt ttttctgcgc gtaatctgct gcttgcaaac aaaaaaacca |
| 7561 | ccgctaccag cggtggtttg tttgccggat caagagctac caactctttt tccgaaggta |
| 7621 | actggcttca gcagagcgca gataccaaat actgtccttc tagtgtagcc gtagttaggc |
| 7681 | caccacttca agaactctgt agcaccgcct acatacctcg ctctgctaat cctgttacca |
| 7741 | gtggctgctg ccagtggcga taagtcgtgt cttaccgggt tggactcaag acgatagtta |
| 7801 | ccggataagg cgcagcggtc gggctgaacg gggggttcgt gcacacagcc cagcttggag |
| 7861 | cgaacgacct acaccgaact gagataccta cagcgtgagc tatgagaaag cgccacgctt |
| 7921 | cccgaaggga gaaaggcgga caggtatccg gtaagcggca gggtcggaac aggagagcgc |
| 7981 | acgagggagc ttccaggggg aaacgcctgg tatctttata gtcctgtcgg gtttcgccac |
| 8041 | ctctgacttg agcgtcgatt tttgtgatgc tcgtcagggg ggcggagcct atggaaaaac |
| 8101 | gccagcaacg cggccttttt acggttcctg gccttttgct ggccttttgc tcacatgttc |
| 8161 | tttcctgcgt tatcccctga ttctgtggat aaccgtatta ccgcctttga gtgagctgat |
| 8221 | accgctcgcc gcagccgaac gaccgagcgc agcgagtcag tgagcgagga agcggaagag |
| 8281 | cgcccaatac gcaaaccgcc tctccccgcg cgttggccga ttcattaatg |
| // | |
| 1376_CCR5.MND.FiX.coFIXpadua.WPRE.pA | |
| (SEQ ID NO: 58) | |
| LOCUS (#1376)\CCR5.MND 7806 bp DNA circular SYN 09-AUG.-2017 | |
| ORIGIN | |
| 1 | cagctgcgcg ctcgctcgct cactgaggcc gcccgggcaa agcccgggcg tcgggcgacc |
| 61 | tttggtcgcc cggcctcagt gagcgagcga gcgcgcagag agggagtggc caactccatc |
| 121 | actaggggtt ccttgtagtt aatgattaac ccgccatgct acttatctac gtagccatgc |
| 181 | tctagcggcc tcggcctctg cataaataaa aaaaattagt cagccatgag cttggacgcg |
| 241 | tttggtgtgg tggcgcctgt agtccccagc cacttggagg ggtgaggtga gaggattgct |
| 301 | tgagcccggg atggtccagg ctgcagtgag ccatgatcgt gccactgcac tccagcctgg |
| 361 | gcgacagagt gagaccctgt ctcacaacaa caacaacaac aacaaaaagg ctgagctgca |
| 421 | ccatgcttga cccagtttct taaaattgtt gtcaaagctt cattcactcc atggtgctat |
| 481 | agagcacaag attttatttg gtgagatggt gctttcatga attcccccaa cagagccaag |
| 541 | ctctccatct agtggacagg gaagctagca gcaaaccttc ccttcactac aaaacttcat |
| 601 | tgcttggcca aaaagagagt taattcaatg tagacatcta tgtaggcaat taaaaaccta |
| 661 | ttgatgtata aaacagtttg cattcatgga gggcaactaa atacattcta ggactttata |
| 721 | aaagatcact ttttatttat gcacagggtg gaacaagatg gattatcaag tgtcaagtcc |
| 781 | aatctatgac atcaattatt atacatcgga gccctgccaa aaaatcaatg tgaagcaaat |
| 841 | cgcagcccgc ctcctgcctc cgctctactc actggtgttc atctttggtt ttgtgggcaa |
| 901 | catgctggtc atcctcatcc tgataaactg caaaaggctg aagagcatga ctgacatcta |
| 961 | cctgctcaac ctggccatct ctgacctgtt tttccttctt actgtcccct tctgggctca |
| 1021 | ctatgctgcc gcccagtggg actttggaaa tacaatgtgt caacgaacag agaaacagga |
| 1081 | gaatatgggc caaacaggat atctgtggta agcagttcct gccccggctc agggccaaga |
| 1141 | acagttggaa cagcagaata tgggccaaac aggatatctg tggtaagcag ttcctgcccc |
| 1201 | ggctcagggc caagaacaga tggtccccag atgcggtccc gccctcagca gtttctagag |
| 1261 | aaccatcaga tgtttccagg gtgccccaag gacctgaaat gaccctgtgc cttatttgaa |
| 1321 | ctaaccaatc agttcgcttc tcgcttctgt tcgcgcgctt ctgctccccg agctctatat |
| 1381 | aagcagagct cgtttagtga accgtcagat cgcctggaga cgccatccac gctgttttga |
| 1441 | cttccataga aggatctcga gatgatcatg gccgagagcc ctggcctgat caccatctgc |
| 1501 | ctgctgggct acctgctgag cgccgagtgc accgtgttcc tggaccacga gaacgccaac |
| 1561 | aagatcctga accggcccaa gagatacaac agcggcaagc tggaggagtt cgtgcagggc |
| 1621 | aacctggaga gggagtgcat ggaggagaag tgcagcttcg aggaggccag ggaagtgttc |
| 1681 | gagaacaccg agcggaccac cgagttctgg aagcagtacg tggacggcga ccagtgcgag |
| 1741 | agcaaccctt gcctgaacgg cggcagctgc aaggacgaca tcaacagcta cgagtgctgg |
| 1801 | tgccctttcg gcttcgaggg caagaactgc gagctggacg tgacctgcaa catcaagaac |
| 1861 | ggccgctgcg agcagttctg caagaacagc gccgacaaca aagtggtgtg tagctgcacc |
| 1921 | gagggctaca gactggccga gaaccagaag agctgcgagc ccgccgtgcc cttcccctgc |
| 1981 | ggcagagtga gcgtgtccca gaccagcaag ctgaccagag ccgagaccgt gttccccgac |
| 2041 | gtggactacg tgaatagcac cgaggccgag accatcctgg acaacatcac ccagagcacc |
| 2101 | cagtccttca acgacttcac cagagttgtg ggcggcgagg acgccaagcc cggccagttc |
| 2161 | ccctggcagg tggtgctgaa cggcaaagtg gatgccttct gcggcggcag catcgtgaac |
| 2221 | gagaagtgga tcgtgacagc cgcccactgc gtggagaccg gcgtgaagat caccgtggtg |
| 2281 | gccggcgaac acaatatcga ggagaccgag cacaccgagc agaagcggaa cgtcatccgg |
| 2341 | attatccccc accacaacta caacgccgcc atcaacaagt acaaccacga catcgccctg |
| 2401 | ctggagctgg acgagcctct ggtgctgaat agctacgtga cccccatctg catcgccgac |
| 2461 | aaggagtaca ccaacatctt cctgaagttc ggcagcggct acgtgtccgg ctggggcaga |
| 2521 | gtgttccaca agggcagaag cgccctggtg ctgcagtacc tgagagtgcc cctggtggac |
| 2581 | agagccacct gcctgttgag caccaagttc accatctaca acaacatgtt ctgcgccggc |
| 2641 | ttccacgagg gcggcagaga cagctgccag ggcgacagcg gcggacccca cgtgaccgaa |
| 2701 | gtggagggca ccagcttcct gaccggcatc atcagctggg gcgaggagtg cgccatgaag |
| 2761 | ggcaagtacg gcatctacac caaagtgagc cggtacgtga actggatcaa ggagaaaacc |
| 2821 | aagctgacct gagtcgacga taatcaacct ctggattaca aaatttgtga aagattgact |
| 2881 | ggtattctta actatgttgc tccttttacg ctatgtggat acgctgcttt aatgcctttg |
| 2941 | tatcatgcta ttgcttcccg tatggctttc attttctcct ccttgtataa atcctggtta |
| 3001 | gttcttgcca cggcggaact catcgccgcc tgccttgccc gctgctggac aggggctcgg |
| 3061 | ctgttgggca ctgacaattc cgtgggtcga ctgctttatt tgtgaaattt gtgatgctat |
| 3121 | tgctttattt gtaaccatta taagctgcaa taaacaagtt aacaacaaca attgcattca |
| 3181 | ttttatgttt caggttcagg gggaggtgtg ggaggttttt taaactctat tttataggct |
| 3241 | tcttctctgg aatcttcttc atcatcctcc tgacaatcga taggtacctg gctgtcgtcc |
| 3301 | atgctgtgtt tgctttaaaa gccaggacgg tcacctttgg ggtggtgaca agtgtgatca |
| 3361 | cttgggtggt ggctgtgttt gcgtctctcc caggaatcat ctttaccaga tctcaaaaag |
| 3421 | aaggtcttca ttacacctgc agctctcatt ttccatacag tcagtatcaa ttctggaaga |
| 3481 | atttccagac attaaagata gtcatcttgg ggctggtcct gccgctgctt gtcatggtca |
| 3541 | tctgctactc gggaatccta aaaactctgc ttcggtgtcg aaatgagaag aagaggcaca |
| 3601 | gggctgtgag gcttatcttc accatcatga ttgtttattt tctcttctgg gctccctaca |
| 3661 | acattgtcct tctcctgaac accttccagg aattctttgg cctgaataat tgcagtagct |
| 3721 | ctaacaggtt ggaccaagct atgcaggtga cagagactct tgggatgacg cactgctgca |
| 3781 | tcaaccccat catctatgcc tttgtcgggg agaagttcag aaactacctc ttagtcttct |
| 3841 | tccaaaagca cattgccaaa cgcttctgca aatgctgttc tattttccag caagaggctc |
| 3901 | ccgagcgagc aagctcagtt tacacccgat ccactgggga gcaggaaata tctgtgggct |
| 3961 | tgtgacacgg actcaagtgg gctggtgacc cagtcagagt tgtgcacatg gcttagtttt |
| 4021 | catacacacc gcggtctaga gcatggctac gtagataagt agcatggcgg gttaatcatt |
| 4081 | aactacaagg aacccctagt gatggagttg gccactccct ctctgcgcgc tcgctcgctc |
| 4141 | actgaggccg ggcgaccaaa ggtcgcccga cgcccgggct ttgcccgggc ggcctcagtg |
| 4201 | agcgagcgag cgcgccagct ggcgtaatag cgaagaggcc cgcaccgatc gcccttccca |
| 4261 | acagttgcgc agcctgaatg gcgaatggcg attccgttgc aatggctggc ggtaatattg |
| 4321 | ttctggatat taccagcaag gccgatagtt tgagttcttc tactcaggca agtgatgtta |
| 4381 | ttactaatca aagaagtatt gcgacaacgg ttaatttgcg tgatggacag actcttttac |
| 4441 | tcggtggcct cactgattat aaaaacactt ctcaggattc tggcgtaccg ttcctgtcta |
| 4501 | aaatcccttt aatcggcctc ctgtttagct cccgctctga ttctaacgag gaaagcacgt |
| 4561 | tatacgtgct cgtcaaagca accatagtac gcgccctgta gcggcgcatt aagcgcggcg |
| 4621 | ggtgtggtgg ttacgcgcag cgtgaccgct acacttgcca gcgccctagc gcccgctcct |
| 4681 | ttcgctttct tcccttcctt tctcgccacg ttcgccggct ttccccgtca agctctaaat |
| 4741 | cgggggctcc ctttagggtt ccgatttagt gctttacggc acctcgaccc caaaaaactt |
| 4801 | gattagggtg atggttcacg tagtgggcca tcgccctgat agacggtttt tcgccctttg |
| 4861 | acgttggagt ccacgttctt taatagtgga ctcttgttcc aaactggaac aacactcaac |
| 4921 | cctatctcgg tctattcttt tgatttataa gggattttgc cgatttcggc ctattggtta |
| 4981 | aaaaatgagc tgatttaaca aaaatttaac gcgaatttta acaaaatatt aacgtttaca |
| 5041 | atttaaatat ttgcttatac aatcttcctg tttttggggc ttttctgatt atcaaccggg |
| 5101 | gtacatatga ttgacatgct agttttacga ttaccgttca tcgattctct tgtttgctcc |
| 5161 | agactctcag gcaatgacct gatagccttt gtagagacct ctcaaaaata gctaccctct |
| 5221 | ccggcatgaa tttatcagct agaacggttg aatatcatat tgatggtgat ttgactgtct |
| 5281 | ccggcctttc tcacccgttt gaatctttac ctacacatta ctcaggcatt gcatttaaaa |
| 5341 | tatatgaggg ttctaaaaat ttttatcctt gcgttgaaat aaaggcttct cccgcaaaag |
| 5401 | tattacaggg tcataatgtt tttggtacaa ccgatttagc tttatgctct gaggctttat |
| 5461 | tgcttaattt tgctaattct ttgccttgcc tgtatgattt attggatgtt ggaatcgcct |
| 5521 | gatgcggtat tttctcctta cgcatctgtg cggtatttca caccgcatat ggtgcactct |
| 5581 | cagtacaatc tgctctgatg ccgcatagtt aagccagccc cgacacccgc caacacccgc |
| 5641 | tgacgcgccc tgacgggctt gtctgctccc ggcatccgct tacagacaag ctgtgaccgt |
| 5701 | ctccgggagc tgcatgtgtc agaggttttc accgtcatca ccgaaacgcg cgagacgaaa |
| 5761 | gggcctcgtg atacgcctat ttttataggt taatgtcatg ataataatgg tttcttagac |
| 5821 | gtcaggtggc acttttcggg gaaatgtgcg cggaacccct atttgtttat ttttctaaat |
| 5881 | acattcaaat atgtatccgc tcatgagaca ataaccctga taaatgcttc aataatattg |
| 5941 | aaaaaggaag agtatgagta ttcaacattt ccgtgtcgcc cttattccct tttttgcggc |
| 6001 | attttgcctt cctgtttttg ctcacccaga aacgctggtg aaagtaaaag atgctgaaga |
| 6061 | tcagttgggt gcacgagtgg gttacatcga actggatctc aacagcggta agatccttga |
| 6121 | gagttttcgc cccgaagaac gttttccaat gatgagcact tttaaagttc tgctatgtgg |
| 6181 | cgcggtatta tcccgtattg acgccgggca agagcaactc ggtcgccgca tacactattc |
| 6241 | tcagaatgac ttggttgagt actcaccagt cacagaaaag catcttacgg atggcatgac |
| 6301 | agtaagagaa ttatgcagtg ctgccataac catgagtgat aacactgcgg ccaacttact |
| 6361 | tctgacaacg atcggaggac cgaaggagct aaccgctttt ttgcacaaca tgggggatca |
| 6421 | tgtaactcgc cttgatcgtt gggaaccgga gctgaatgaa gccataccaa acgacgagcg |
| 6481 | tgacaccacg atgcctgtag caatggcaac aacgttgcgc aaactattaa ctggcgaact |
| 6541 | acttactcta gcttcccggc aacaattaat agactggatg gaggcggata aagttgcagg |
| 6601 | accacttctg cgctcggccc ttccggctgg ctggtttatt gctgataaat ctggagccgg |
| 6661 | tgagcgtggg tctcgcggta tcattgcagc actggggcca gatggtaagc cctcccgtat |
| 6721 | cgtagttatc tacacgacgg ggagtcaggc aactatggat gaacgaaata gacagatcgc |
| 6781 | tgagataggt gcctcactga ttaagcattg gtaactgtca gaccaagttt actcatatat |
| 6841 | actttagatt gatttaaaac ttcattttta atttaaaagg atctaggtga agatcctttt |
| 6901 | tgataatctc atgaccaaaa tcccttaacg tgagttttcg ttccactgag cgtcagaccc |
| 6961 | cgtagaaaag atcaaaggat cttcttgaga tccttttttt ctgcgcgtaa tctgctgctt |
| 7021 | gcaaacaaaa aaaccaccgc taccagcggt ggtttgtttg ccggatcaag agctaccaac |
| 7081 | tctttttccg aaggtaactg gcttcagcag agcgcagata ccaaatactg tccttctagt |
| 7141 | gtagccgtag ttaggccacc acttcaagaa ctctgtagca ccgcctacat acctcgctct |
| 7201 | gctaatcctg ttaccagtgg ctgctgccag tggcgataag tcgtgtctta ccgggttgga |
| 7261 | ctcaagacga tagttaccgg ataaggcgca gcggtcgggc tgaacggggg gttcgtgcac |
| 7321 | acagcccagc ttggagcgaa cgacctacac cgaactgaga tacctacagc gtgagctatg |
| 7381 | agaaagcgcc acgcttcccg aagggagaaa ggcggacagg tatccggtaa gcggcagggt |
| 7441 | cggaacagga gagcgcacga gggagcttcc agggggaaac gcctggtatc tttatagtcc |
| 7501 | tgtcgggttt cgccacctct gacttgagcg tcgatttttg tgatgctcgt caggggggcg |
| 7561 | gagcctatgg aaaaacgcca gcaacgcggc ctttttacgg ttcctggcct tttgctggcc |
| 7621 | ttttgctcac atgttctttc ctgcgttatc ccctgattct gtggataacc gtattaccgc |
| 7681 | ctttgagtga gctgataccg ctcgccgcag ccgaacgacc gagcgcagcg agtcagtgag |
| 7741 | cgaggaagcg gaagagcgcc caatacgcaa accgcctctc cccgcgcgtt ggccgattca |
| 7801 | ttaatg |

Compositions, Methods of Making Cells, and Cells that Secrete a Macromolecule for Promoting Tolerance of a Protein or Peptide, Survival of a B Cell or Engraftment of a Cell, Such as a B Cell.

In another aspect a method of making plasma cells or plasma cell precursors that expresses a second macromolecule, such as protein, or peptide is provided, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells and/or any mixtures or combinations thereof. In some alternatives, second macromolecule is for inducing tolerance of a protein or peptide for therapeutic treatment or for inducing engraftment. In some alternatives, the second macromolecule is a protein. The protein may be BAFF, APRIL, IFN-alpha, IL-10 or IL-6. In some alternatives the protein is a viral vector capsid protein. In some alternatives, the viral vector capsid protein comprises at least one the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3). As described herein, B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof. In some alternatives, the method further comprises purifying the plasma cells or plasma cell precursors against CD138. In some alternatives, purifying comprises selecting the plasma cells by an anti-CD138 antibody for targeted removal of the plasma cells. In some alternatives, the purifying comprises using anti-CD138 beads for plasma cell selection.

In another aspect, a method of making a long lived plasma cell is provided, wherein the method comprises: isolating B cells; activating the B cells; a first round of genome editing of the B cells for macromolecule expression, such as a protein, protein mimetic or a peptide in the absence of viral integration; expanding the B cells; and differentiating the B cells. In some alternatives, the macromolecule is a protein, protein mimetic or peptide. In some alternatives, macromolecule is for inducing tolerance of a protein or peptide for therapeutic treatment or for inducing engraftment. In some alternatives, the macromolecule is a protein. The protein may be BAFF, APRIL, IFN-alpha, IL-10 or IL-6. In some alternatives the protein is a viral vector capsid protein. In some alternatives, the viral vector capsid protein comprises one or more of following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3). In some alternatives, the method further comprises purifying the plasma cells or plasma cell precursors against CD138. In some alternatives, purifying comprises selecting the plasma cells by an anti-CD138 antibody for targeted removal of the plasma cells. In some alternatives, the purifying comprises using anti-CD138 beads for plasma cell selection.

In another aspect, a plasma cell that expresses a macromolecule, such as a heterologous protein, protein mimetic or a peptide is provided. In some alternatives, the cell is a B cell. In some alternatives, the macromolecule is for inducing tolerance or for inducing engraftment. In some alternatives, the macromolecule is a protein. The protein may be BAFF, APRIL, IFN-alpha, IL-10 or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3). In some alternatives, the plasma cell expresses CD20.

In some alternatives a plasma cell is provided, wherein the plasma cell expresses a molecule, such as a macromolecule, protein, or peptide. The plasma cell or plasma cell precursors are manufactured by any one of the alternatives herein is provided. The method can comprise the steps: (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the developing of the B cells is performed after the B cells are arrested at a specific phase of development and/or activating the B cells such that the B cells are permissive for recombination without further B cell differentiation. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a CAS nuclease. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cell. In some alternatives, the method to prevent somatic hypermutation of an antibody locus in the B cell comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding of the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or any other length defined by a range that is set forth by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element and/or a synthetic engineered protein. In some alternatives, the protein is IFN-alpha, IL-6, Factor VIII, Factor IX, SERPING1 or SERPINA1. In some alternatives, the enzyme is ADAMTS13, LIPA, GLA, or ALPL. In some alternatives, the protein is a receptor antagonist that is used for the treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, a therapeutic antibody or binding portion thereof is used e.g., an antibody or binding portion thereof that is specific for a protein or other molecule expressed in an autoimmune disorder, autoinflammatory disorder, immune dysregulation and/or cancer. In some alternatives, the antibody or binding portion thereof is an anti-IL-1 monoclonal antibody, anti-TNF antibody, anti-IL-33 antibody or anti-C5 antibody or a binding portion thereof. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies. In some alternatives, the protein is an engineered protein, wherein the engineered protein is protective in viral, fungal, parasitic and/or bacterial infection. In some alternatives, the engineered protein comprises an antibody or a binding portion thereof, wherein the antibody or binding portion thereof is specific for an antigen that is expressed on a virus, fungus, parasite or bacteria. In some alternatives, the protein comprises an antibody or binding portion thereof, wherein the antibody or binding portion thereof is specific for an antigen that is expressed in a viral, fungal, parasitic or bacterial infection. In some alternatives, the B cells comprise B cell precursors such as hematopoietic stem cells (HSCs), multipotent progenitor (MPP) cells, lymphoid progenitor (CLP) cells, naïve B cells, GC B cell, plasmablast, early pro-B cells, late pro-B cells, large pre-B cells, small pre-B cells, immature B cells, T1 B cells, T2 B cells, marginal zone B cells, mature B cells and memory B cells. In some alternatives, the molecule is a macromolecule, protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein comprises a cofactor for enhanced enzymatic activity. In some alternatives the method of making the a long lived plasma cell comprises the steps: isolating B cells; activating the B cells; a first round of genome editing of the B cells for molecule expression, such as a protein, protein mimetic or a peptide in the absence of viral integration; expanding the B cells; and differentiating the B cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the B cells from the isolating step comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody or binding portion thereof is an anti-IL-1 monoclonal antibody, anti-TNF antibody, anti-IL-33 antibody or anti-C5 antibody or a binding portion of any of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNABs). In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from a group selected from macromolecules, proteins, protein mimetics and peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the isolating is performed by negative selection isolation of hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the method further comprises purifying the plasma cells or plasma cell precursors against CD138. In some alternatives, purifying comprises selecting the plasma cells by an anti-CD138 antibody for targeted removal of the plasma cells. In some alternatives, the purifying comprises using anti-CD138 beads for plasma cell selection.

In another aspect, a composition comprising the plasma cell manufactured by any one of the alternatives provided herein or comprising the cell of any one of the alternatives provide herein, is provided. The plasma cell or plasma cell precursors are manufactured by any one of the alternatives herein is provided. The method can comprise the steps: (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the method further comprises purifying the plasma cells or plasma cell precursors against CD138. In some alternatives, purifying comprises selecting the plasma cells by an anti-CD138 antibody for targeted removal of the plasma cells. In some alternatives, the purifying comprises using anti-CD138 beads for plasma cell selection.In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the developing of the B cells is performed after the B cells are arrested at a specific phase of development and/or activating the B cells such that the B cells are permissive for recombination without further B cell differentiation. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cells. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a CAS nuclease. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding of the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or any other length defined by a range that is set forth by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element and/or a synthetic engineered protein. In some alternatives, the protein is IFN-alpha, IL-6, Factor VIII, Factor IX, SERPING1 or SERPINA1. In some alternatives, the enzyme is ADAMTS13, LIPA, GLA, or ALPL. In some alternatives, the protein is a receptor antagonist that is used for the treatment or amelioration of periodic fever or autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, a therapeutic antibody or binding portion thereof is used e.g., an antibody or binding portion thereof that is specific for a protein or other molecule expressed in an autoimmune disorder, autoinflammatory disorder, immune dysregulation and/or cancer. In some alternatives, the antibody or binding portion thereof is an anti-IL-1 monoclonal antibody, anti-TNF antibody, anti-IL-33 antibody or anti-C5 antibody or a binding portion thereof. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies. In some alternatives, the protein is an engineered protein, wherein the engineered protein is protective in viral, fungal, parasitic and/or bacterial infection. In some alternatives, the engineered protein comprises an antibody or a binding portion thereof, wherein the antibody or binding portion thereof is specific for an antigen that is expressed on a virus, fungus, parasite or bacteria. In some alternatives, the protein comprises an antibody or binding portion thereof, wherein the antibody or binding portion thereof is specific for an antigen that is expressed in a viral, fungal, parasitic or bacterial infection. In some alternatives, the B cells comprise B cell precursors such as hematopoietic stem cells (HSCs), multipotent progenitor (MPP) cells, lymphoid progenitor (CLP) cells, naïve B cells, GC B cell, plasmablast, early pro-B cells, late pro-B cells, large pre-B cells, small pre-B cells, immature B cells, T1 B cells, T2 B cells, marginal zone B cells, mature B cells and memory B cells. In some alternatives, the molecule is a macromolecule, protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein comprises a cofactor for enhanced enzymatic activity. In some alternatives the method of making the a long lived plasma cell comprises the steps: isolating B cells; activating the B cells; a first round of genome editing of the B cells for molecule expression, such as a protein, protein mimetic or a peptide in the absence of viral integration; expanding the B cells; and differentiating the B cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the B cells from the isolating step comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody or binding portion thereof is an anti-IL-1 monoclonal antibody, anti-TNF antibody, anti-IL-33 antibody or anti-C5 antibody or a binding portion of any of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNABs). In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from a group selected from macromolecules, proteins, protein mimetics and peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the isolating is performed by negative selection isolation of hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the plasma cell is derived from a B cell. In some alternatives, the plasma cell expresses a molecule, such as a heterologous protein, protein mimetic or a peptide. In some alternatives, the cell is a B cell precursor. In some alternatives herein, the method further comprises purifying the plasma cells or plasma cell precursors by positive selection against CD138. In some alternatives herein, purifying comprises selecting the plasma cells by an anti-CD138 antibody for targeted removal of the plasma cells. In some alternatives herein, the purifying comprises using anti-CD138 beads for plasma cell selection. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells and/or any mixtures or combinations thereof.. In some alternatives, the B cell expresses a macromolecule is for inducing tolerance or for inducing engraftment. In some alternatives, the macromolecule is a protein. The protein may be BAFF, APRIL, IFN-alpha, IL-10 or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3).

In another aspect, a method of expressing a molecule, such as a macromolecule in a subject in need is provided, wherein the method comprises: providing or administrating a plasma cell manufactured by any one of the alternative methods provided herein, the plasma cell that expresses molecule of any one of any one of the alternative plasma cells provided herein, or the composition of any one of the alternatives described herein, into the subject in need and administering a second B cell or second plasma cell, wherein the second B cell or second plasma cell expresses a second macromolecule, wherein the second macromolecule promotes inducing tolerance of a protein or peptide for therapy or induces engraftment. In some alternatives, the second plasma cell is derived from a B cell. In some alternatives, the second plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells and/or any mixtures or combinations thereof.. In some alternatives, the second B cell expresses a second macromolecule for inducing tolerance of a protein or peptide for therapy or induces or promotes engraftment. In some alternatives, the second macromolecule is a protein. The protein may be BAFF, APRIL, IFN-alpha, IL-10 or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3).

In another aspect, a method of treating, ameliorating or inhibiting a disease, such as a cancer, in a subject in need is provided, the method comprises administering a plasma cell manufactured by any one of the alternative methods herein or the plasma cell that expresses a molecule, such as a macromolecule manufactured by any one of the alternative methods herein, or the composition of any one of the alternative compositions herein into the subject in need and administering a second B cell or second plasma cell, wherein the second B cell or second plasma cell expresses a second macromolecule, wherein the second macromolecule promotes inducing tolerance of a protein or peptide for therapy or induces engraftment. In some alternatives, the second plasma cell is derived from a B cell. In some alternatives, the second plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells and/or any mixtures or combinations thereof.. In some alternatives, the second B cell expresses a second macromolecule for inducing tolerance of a protein or peptide for therapy or induces or promotes engraftment. In some alternatives, the second macromolecule is a protein. The protein may be BAFF, APRIL, IFN-alpha, IL-10 or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3). In some alternatives, the method comprises monitoring of the subject following plasma cell transplantation. In some alternatives, the subject is determined to no longer need treatment or therapy and the method further comprises administering rituxan® or an anti-CD20 antibody or binding portion thereof for targeted removal of the plasma cells.

In another aspect, a method of expressing a molecule, such as a macromolecule in a subject of need is provided, wherein the method comprises: (a) isolating a first population of B cells; (b) developing the first population of B cells; (c) performing a first round of genome editing of the first population of B cells for protein expression in absence of viral integration; (d) expanding the first population of B cells; differentiating the first population of B cells, optionally, after step (c) or (d), thereby producing plasma cells that express a protein; (e) administering the first population of B cells into the subject in need and (f) administering a second population of B cells into the subject in need, wherein the second population of B cells expresses a second macromolecule for inducing tolerance of a protein or peptide for therapy or induces or promotes engraftment. In some alternatives, the second macromolecule is a protein. The protein may be BAFF, APRIL, IFN-alpha, IL-10 or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3). In some alternatives, the method further comprises purifying the plasma cells or plasma cell precursors by positive selection against CD138. In some alternatives, purifying comprises selecting the plasma cells by an anti-CD138 antibody for targeted removal of the plasma cells. In some alternatives, the purifying comprises using anti-CD138 beads for plasma cell selection.

In another aspect, a method of ameliorating a disease in a subject in need is provided, the method comprising: administering a plasma cell manufactured by any one of the alternatives herein or the plasma cell that expresses a molecule, such as a macromolecule manufactured by any one of the alternatives herein, or the composition of the alternatives herein into the subject in need and administering a second B cell or second plasma cell, wherein the second B cell or second plasma cell expresses a second macromolecule, wherein the second macromolecule promotes inducing tolerance of a protein or peptide for therapy or induces engraftment. In some alternatives, the second plasma cell is derived from a B cell. In some alternatives, the second plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells and/or any mixtures or combinations thereof.. In some alternatives, the second B cell expresses a second macromolecule for inducing tolerance of a protein or peptide for therapy or induces or promotes engraftment. In some alternatives, the second macromolecule is a protein. The protein may be BAFF, APRIL, IFN-alpha, IL-10 or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3). In some alternatives, the administering is performed by adoptive transfer. In some alternatives, the subject has received a stem cell administration or solid organ transplantation. In some alternatives, the subject suffers from influenza, parainfluenza, rhinovirus, respiratory syncytial virus, HIV, or other pathogenic bacteria, virus, fungus or parasite. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives a second B cell expressing a second macromolecule is provided, wherein the second B cell provides the second macromolecule for inducing tolerance or for inducing engraftment. In some alternatives, the second macromolecule is a protein. The protein may be BAFF, APRIL, IFN-alpha, IL-10, IFN-alpha or IL-6. In some alternatives the protein is a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3).

In some alternatives, a method of inducing tolerance of a peptide useful for therapeutic application, secreted by a B cell in a subject in need in vivo and/or increasing engraftment of the B cell in a subject in need is provided, the method comprises A) isolating a first population of B cells; B) performing a first round of genome editing of the first population of B cells for protein or peptide expression in absence of viral integration, wherein the performing is under conditions of rapid B cell expansion; C) expanding the first population of B cells; D) differentiating the first population of B cells, optionally, after step (c) or (d), thereby producing an activated first population of B cells or naïve activated first population of B cells that express the molecule; E) providing or administering the first population of B cells that expresses the peptide into a subject in need; and F) administering a second population of B cells that expresses a macromolecule for tolerance, survival of a B cell or engraftment. In some alternatives, the therapeutic application is treatment of cancer. In some alternatives, the therapeutic application is immunotherapeutic application. In some alternatives, the cancer is a solid tumor. In some alternatives, the peptide is an anti-PD1 antibody or a PDL-1 ligand. In some alternatives, the second B cell expresses BAFF, APRIL, IFN-alpha, IL-10 or IL-6. In some alternatives, the second B cell expresses a second peptide that is expressed and presented as a MHC class II molecule on a surface of the B cells.

In some alternatives, a method of inducing tolerance of a peptide for immunotherapeutic application, secreted by a B cell in a subject in need in vivo and/or increasing engraftment or survival of the B cell in a subject in need, the method comprising: A) isolating a first population of B cells; B) performing a first round of genome editing of the first population of B cells for protein or peptide expression in absence of viral integration, wherein the performing is under conditions of rapid B cell expansion; C) expanding the first population of B cells; D) differentiating the first population of B cells, optionally, after step (c) or (d), thereby producing an activated first population of B cell or naïve activated first population of B cells that express the protein or peptide; E) providing or administering the first population of B cells that expresses the protein or peptide into a subject in need F) providing or administering a second population of B cells that expresses a macromolecule, wherein the second population of B cells allows tolerance of the protein or peptide and/or promotes engraftment and/or survival. In some alternatives, the first population of B cells and the second population of B cells are co-administered. In some alternatives, the macromolecule comprises BAFF, APRTL, IFN-alpha, IL-10 or IL-6. In some alternatives, the protein comprises an enzyme, neutralizing antibody or a binding portion thereof, cytokine, cytokine receptor, complement protein, inhibitory protein, anti-fibrotic molecule, therapeutic antibody or a binding portion thereof, anti-thrombotic molecule, glucose response element or monoclonal antibody or a binding portion thereof. In some alternatives, the protein is SERPING1 or SERPINA1. In some alternatives, the protein is a receptor antagonist for the treatment or inhibition of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angioedema. In some alternatives, the protein is an anti-fibrotic molecule, wherein the anti-fibrotic molecule is SCGB1A1. In some alternatives, the protein comprises therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof.

In some alternatives, a composition comprising the plasma cell manufactured by any one of the alternatives herein and a second B cell, wherein the second B cell secretes a macromolecule for inducing tolerance of a peptide for immunotherapeutic application or for inducing engraftment of the plasma cell manufactured by any one of the alternatives herein is provided. In some alternatives, the macromolecule expressed by the second B cell comprises IFN-alpha, BAFF, APRIL, IL-10 or IL-6. In some alternatives, the second B cell expresses a viral vector capsid protein. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) and/or AAV9 (VP1, VP2 and VP3).

TABLE 4
Additional guide sequences that are used for some of the alternatives herein.

| Sense/ | | |
|---|---|---|
| Broad rank | Guide Sequence - RNA | Guide sequence - mod & linker |
| Antisense | GGACTCCCCAGAAAAGCAAA | mG*mG*mA*rCrUrCrCrCrCrArGrArArArArGrCrArArArGrUrUrUrUrArGrArGr |
| (−) | (SEQ ID NO: 63) | CrUrArU*mG*mC*mU (SEQ ID NO: 88) |
| Sense | AAGAACCATTTGCTTTTCTG | mA*mA*mG*rArArCrCrArUrUrUrGrCrUrUrUrUrCrUrGrGrUrUrUrUrArGrArGr |
| (−) | (SEQ ID NO: 64) | CrUrArU*mG*mC*mU (SEQ ID NO: 89) |
| Sense | TTTGCTTTICTGGGGAGTCC | mU*mU*mU*rGrCrUrUrUrUrCrUrGrGrGrGrArGrUrCrCrGrUrUrUrUrArGrArGr |
| (−) | (SEQ ID NO: 65) | CrUrArU*mG*mC*mU (SEQ ID NO: 90) |
| Sense | ATTAAGGCTGTTCATGTGAA | mA*mU*mU*rArArGrGrCrUrGrUrUrCrArUrGrUrGrArArGrUrUrUrUrArGrArGr |
| (−) | (SEQ ID NO: 66) | CrUrArU*mG*mC*mU (SEQ ID NO: 91) |
| Antisense | CCGGCGCGGCAGGCGCATGG | mC*mC*mG*rGrCrGrCrGrGrCrArGrGrCrGrCrArUrGrGrGrUrUrUrUrArGrArGr |
| (−) | (SEQ ID NO: 67) | CrUrArU*mG*mC*mU (SEQ ID NO: 92) |
| Antisense | AGCCCCAGCGCGGCCCGGCG | mA*mG*mC*rCrCrCrArGrCrGrCrGrGrCrCrCrGrGrCrGrGrUrUrUrUrArGrArGr |
| (−) | (SEQ ID NO: 68) | CrUrArU*mG*mC*mU (SEQ ID NO: 93) |
| Sense | TGCCGCGCCGGGCCGCGCTG | mU*mG*mC*rCrGrCrGrCrCrGrGrGrCrCrGrCrGrCrUrGrGrUrUrUrUrArGrArGr |
| (−) | (SEQ ID NO: 69) | CrUrArU*mG*mC*mU (SEQ ID NO: 94) |
| Antisense | AAGCGGCAGGAGCCCCAGCG | mA*mA*mG*rCrGrGrCrArGrGrArGrCrCrCrCrArGrCrGrGrUrUrUrUrArGrArGr |
| (−) | (SEQ ID NO: 70) | CrUrArU*mG*mC*mU (SEQ ID NO: 95) |
| Sense | AGGTAGACAATTGCAGCCTG | mA*mG*mG*rUrArGrArCrArArUrUrGrCrArGrCrCrUrGrGrUrUrUrUrArGrArGr |
| (1) | (SEQ ID NO: 71) | CrUrArU*mG*mC*mU (SEQ ID NO: 96) |
| Sense | TCCCTACAGACAGAGCCACA | mU*mC*mC*rCrUrArCrArGrArCrArGrArGrCrCrArCrArGrUrUrUrUrArGrArGr |
| (12) | (SEQ ID NO: 72) | CrUrArU*mG*mC*mU (SEQ ID NO: 97) |
| Sense | AGATGTTGTCCTGACACTTG | mA*mG*mA*rUrGrUrUrGrUrCrCrUrGrArCrArCrUrUrGrGrUrUrUrUrArGrArGr |
| (3) | (SEQ ID NO: 73) | CrUrArU*mG*mC*mU (SEQ ID NO: 98) |
| Sense | GCCTTCACCATGAAGTCCAG | mG*mC*mC*rUrUrCrArCrCrArUrGrArArGrUrCrCrArGrGrUrUrUrUrArGrArGr |
| (16) | (SEQ ID NO: 74) | CrUrArU*mG*mC*mU (SEQ ID NO: 99) |
| Sense | TTCAACTGCTCATCAGATGG | mU*mU*mC*rArArCrUrGrCrUrCrArUrCrArGrArUrGrGrGrUrUrUrUrArGrArGr |
| (33) | (SEQ ID NO: 75) | CrUrArU*mG*mC*mU (SEQ ID NO: 100) |
| Antisense | GGCCAAAGTACAGTGGAAGG | mG*mG*mC*rCrArArArGrUrArCrArGrUrGrGrArArGrGrGrUrUrUrUrArGrArGr |
| (4) | (SEQ ID NO: 76) | CrUrArU*mG*mC*mU (SEQ ID NO: 100 |
| Sense | CACGCTGCTCGTATCCGACG | mC*mA*mC*rGrCrUrGrCrUrCrGrUrArUrCrCrGrArCrGrGrUrUrUrUrArGrArGr |
| (4) | (SEQ ID NO: 77) | CrUrArU*mG*mC*mU (SEQ ID NO: 102) |
| Antisense | CCTTCCAAGGACGTCATGCA | mC*mC*mU*rUrCrCrArArGrGrArCrGrUrCrArUrGrCrArGrUrUrUrUrArGrArGr |
| (5) | (SEQ ID NO: 78) | CrUrArU*mG*mC*mU (SEQ ID NO: 103) |
| Antisense | CAGCCTTAATAAAAACCGCC | mC*mA*mG*rCrCrUrUrArArUrArArArArArCrCrGrCrCrGrUrUrUrUrArGrArGr |
| (4) | (SEQ ID NO: 79) | CrUrArU*mG*mC*mU (SEQ ID NO: 104) |
| Sense | TTCTTCCGAAGATCCTAATG | mU*mU*mC*rUrUrCrCrGrArArGrArUrCrCrUrArArUrGrGrUrUrUrUrArGrArGr |
| (12) | (SEQ ID NO: 80) | CrUrArU*mG*mC*mU (SEQ ID NO: 105) |
| Antisense | GTCAGGATAGCAGGCATCTG | mG*mU*mC*rArGrGrArUrArGrCrArGrGrCrArUrCrUrGrGrUrUrUrUrArGrArGr |
| (32) | (SEQ ID NO: 81) | CrUrArU*mG*mC*mU (SEQ ID NO: 100 |
| Antisense | TACTGGCTCCACTTCTCGAG | mU*mA*mC*rUrGrGrCrUrCrCrArCrUrUrCrUrCrGrArGrGrUrUrUrUrArGrArGr |
| (3) | (SEQ ID NO: 82) | CrUrArU*mG*mC*mU (SEQ ID NO: 107) |
| Sense | GATATTGATATACTTCCTAG | mG*mA*mU*rArUrUrGrArUrArUrArCrUrUrCrCrUrArGrGrUrUrUrUrArGrArGr |
| (20) | (SEQ ID NO: 83) | CrUrArU*mG*mC*mU (SEQ ID NO: 108) |
| Antisense | ACCCAAAGGGGTCTCAAAGG | mA*mC*mC*rCrArArArGrGrGrGrUrCrUrCrArArArGrGrGrUrUrUrUrArGrArGr |
| (2) | (SEQ ID NO: 84) | CrUrArU*mG*mC*mU (SEQ ID NO: 109) |
| Antisense | CAGCGTCTTAGCACCCAAAG | mC*mA*mG*rCrGrUrCrUrUrArGrCrArCrCrCrArArArGrGrUrUrUrUrArGrArGr |
| (10) | (SEQ ID NO: 85) | CrUrArU*mG*mC*mU (SEQ ID NO: 110) |
| Sense | CGAATGCAATCAGATGCTAG | mC*mG*mA*rArUrGrCrArArUrCrArGrArUrGrCrUrArGrGrUrUrUrUrArGrArGr |
| (3) | (SEQ ID NO: 86) | CrUrArU*mG*mC*mU (SEQ ID NO: 111) |
| Antisense | ACCGAGACAGTCGGGACCGT | mA*mC*mC*rGrArGrArCrArGrUrCrGrGrGrArCrCrGrUrGrUrUrUrUrArGrArGr |
| (4) | (SEQ ID NO: 87) | CrUrArU*mG*mC*mU (SEQ ID NO: 112) |

As shown the modifications are: m - 2'O methyl-modified bases, r - standard RNA bases* - phosphorothioate bond

In some alternatives, a method of making plasma cells or plasma cell precursors that express a molecule is provided, wherein the method comprises: (a)isolating B cells, (b) developing the B cells, (c) performing a first round of genome editing of the B cells for protein expression in absence of viral integration, (d) expanding the B cells and (e) differentiating the B cells, optionally, after step (c) or (d), thereby producing plasma cells or plasma cell precursors that express the molecule. In some alternatives, the molecule is a protein, protein mimetic or peptide. In some alternatives, the molecule is Factor VIII, Factor IX, SERPING1, SERPINA1, complement inhibitory protein, Factor H, Factor I, a C1 inhibitor, an anti-fibrotic molecule, SCGBlA1, a therapeutic antibody or a binding portion thereof, an anti-IL-1 monoclonal antibody, an anti-TNF antibody, an anti-IL-33 antibody, an anti-C5 antibody, an anti-thrombotic molecule, APLN, an anti-PCSK9 inhibitory antibody or binding portion thereof, a neutralizing HIV-1 antibody (bNAbs) or binding portion thereof, IFN-alpha, BAFF, APRIL, IL-10, IL-6, ADAMTS13, LIPA, GLA or ALPL. In some alternatives, the developing of the B cells is performed after the B cells are arrested at a specific phase of development or activating the B cells such that the B cells are permissive for recombination without further B cell differentiation, wherein the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration, and wherein performing the first round of genome editing of the B cells for protein expression is performed by introduction of a single stranded nucleic acid. In some alternatives, the first round of genome editing is performed by an RNA and protein based transfection and, wherein the first round of genome editing comprises delivering a nuclease, wherein the nuclease targets at least one genetic loci in the B cell and, wherein the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the first round of genome editing comprises transducing the B cell with a recombinant adeno-associated virus vector to serve as a donor template for homologous recombination into a candidate genetic loci. In some alternatives, the method further comprises preventing somatic hypermutation of an antibody locus in the B cell. In some alternatives, the method further comprises selectively increasing a proportion of gene edited B cells and, wherein the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, step (a) further comprises removing IgM positive cells. In some alternatives, the method further comprises introducing a nucleic acid, wherein the nucleic acid encodes at least one cell surface protein. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 or IL-15, wherein the plasmablast differentiation step is performed in the presence any combination of IL-2, IL-6, IL-10 or IL-15 or wherein the plasma cell differentiation step is performed in the presence any combination of IL-6, IL-15, APRIL or IFNα. In some alternatives, the plasma cells are long lived plasma cells. In some alternatives, the method further comprises purifying the plasma cells by positive selection against CD138.

In some alternatives, a composition comprising a plasma cell, which expresses a molecule, wherein said molecule is a heterologous protein, protein mimetic or a peptide is provided. In some alternatives, the molecule comprises an enzyme, neutralizing antibody or binding portion thereof, cytokine, cytokine receptor, complement protein, inhibitory protein, anti-fibrotic molecule, therapeutic antibody or binding portion thereof, anti-thrombotic molecule, glucose response element, or a monoclonal antibody or binding portion thereof. In some alternatives, the composition further comprises a second B cell, wherein the second B cell secretes a molecule that induces tolerance of a peptide or that induces engraftment of the plasma cell.

In some alterantives, a method of expressing a molecule in a subject is provided wherein the method comprises administering the composition of anyone of the alternatives to the subject. In some alterantives, the composition comprises a plasma cell, which expresses a molecule, wherein said molecule is a heterologous protein, protein mimetic or a peptide is provided. In some alternatives of the composition, the molecule comprises an enzyme, neutralizing antibody or binding portion thereof, cytokine, cytokine receptor, complement protein, inhibitory protein, anti-fibrotic molecule, therapeutic antibody or binding portion thereof, anti-thrombotic molecule, glucose response element, or a monoclonal antibody or binding portion thereof. In some alternatives, the composition further comprises a second B cell, wherein the second B cell secretes a molecule that induces tolerance of a peptide or that induces engraftment of the plasma cell. In some alterantives, the subject has received a stem cell administration or a solid organ transplantation or is a subject identified or selected as one

To Receive a Stem Cell Administration or a Solid Organ Transplantation or, Wherein the Subject has an enzyme deficiency, pulmonary fibrosis, an autoimmune disorder, immune dysregulation, cancer, diabetes, HIV or hypercholesterolemia. More alternatives

In some alternatives, a method of making plasma cells or plasma cell precursors that expresses a molecule, such as a macromolecule, protein, or peptide is provided, wherein the method comprises (a) isolating B cells; (b) developing the B cells; (c) performing a first round of genome editing of the B cells for expression of a molecule in absence of viral integration; (d) expanding the B cells; and (e) differentiating the B cells, optionally, after step (c) or (d), thereby producing plasma cells or plasma cell precursors that express the molecule. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the protein comprises an enzyme, monoclonal antibody or a binding portion thereof, neutralizing antibodies or a binding portions thereof, therapeutic antibodies or binding portions thereof, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGBIAI. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs), or a binding portion thereof. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed by a virus, fungus, parasite or bacteria. In some alternatives, the protein comprises an antibody or a binding portion thereof, wherein the antibody or a binding portion thereof is specific for an antigen that is expressed on a viral, fungal, parasitic or bacterial infection. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the developing of the B cells is performed after the B cells are arrested at a specific phase of development and/or activating the B cells such that the B cells are permissive for recombination without further B cell differentiation. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are patient derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a CAS nuclease. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cell. In some alternatives, the method to prevent somatic hypermutation of an antibody locus in the B cell comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cell short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding of the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or any other length defined by a range that is set forth by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL-2, 50 ng/ml of IL-10 and 10 ng/ml of IL-15 for two days. In some alternatives, the cells were washed with 1×PBS and seeded in medium with IL-6 (50 ng/ml), IL-15 (10 ng/ml) and human interferon-α 2B (100 U/ml, Sigma-Aldrich®) for three days to stimulate plasma cell differentiation. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element and/or a synthetic engineered protein. In some alternatives, the protein is IFN-alpha, IL-6, Factor VIII, Factor IX, SERPING1 or SERPINA1. In some alternatives, the enzyme is ADAMTS13, LIPA, GLA, or ALPL. In some alternatives, the protein is a receptor antagonist that is used for the treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, a therapeutic antibody or binding portion thereof is used e.g., an antibody or binding portion thereof that is specific for a protein or other molecule expressed in an autoimmune disorder, autoinflammatory disorder, immune dysregulation and/or cancer. In some alternatives, the antibody or binding portion thereof is an anti-IL1 monoclonal antibody, anti-TNF antibody, anti-IL33 antibody or anti-C5 antibody or a binding portion thereof. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies. In some alternatives, the protein is an engineered protein, wherein the engineered protein is protective in viral, fungal, parasitic and/or bacterial infection. In some alternatives, the engineered protein comprises an antibody or a binding portion thereof, wherein the antibody or binding portion thereof is specific for an antigen that is expressed on a virus, fungus, parasite or bacteria. In some alternatives, the protein comprises an antibody or binding portion thereof, wherein the antibody or binding portion thereof is specific for an antigen that is expressed in a viral, fungal, parasitic or bacterial infection. In some alternatives, the B cells comprise B cell precursors such as hematopoietic stem cells (HSCs), multipotent progenitor (MPP) cells, lymphoid progenitor (CLP) cells, naïve B cells, GC B cell, plasmablast, early pro-B cells, late pro-B cells, large pre-B cells, small pre-B cells, immature B cells, T1 B cells, T2 B cells, marginal zone B cells, mature B cells and/or memory B cells. In some alternatives, the molecule is a macromolecule, protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein comprises a cofactor for enhanced enzymatic activity. In some alternatives, the method further comprises purifying the plasma cells or plasma cell precursors after step e) by positive selection against CD138. In some alternatives, purifying comprises selecting the plasma cells by an anti-CD138 antibody for targeted removal of the plasma cells. In some alternatives, the purifying comprises using anti-CD138 beads for plasma cell selection.

In some alternatives, a method of making a long lived plasma cell is provided, wherein the method comprises: isolating B cells; activating the B cells; a first round of genome editing of the B cells for molecule expression, such as a protein, protein mimetic or a peptide in the absence of viral integration; expanding the B cells; and differentiating the B cells, thereby producing the long lived plasma cell. In some alternatives, the method further comprises selecting the long lived plasma cell by CD138 selection of the plasma cells. In some alternatives, the macromolecule is a protein, protein mimetic or peptide. In some alternatives, the protein, protein mimetic, or peptide comprises natural amino acids and/or unnatural amino acids. In some alternatives, the macromolecule comprises a carbohydrate or lipid moiety. In some alternatives, the macromolecule comprises a cofactor. In some alternatives, the protein comprises a hydrophobic group for membrane localization. In some alternatives, the protein is acylated. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for the treatment or inhibition of periodic fever or autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL-1 monoclonal antibody, anti-TNF antibody, anti-IL-33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the B cells from the isolating step comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cell with a recombinant adeno-associated virus vector for homologous recombination into a candidate genetic loci. In some alternatives, the method further comprises applying methods to prevent somatic hypermutation of an antibody locus and other target loci in the B cell. In some alternatives, the method to prevent somatic hypermutation of an antibody locus in the B cell comprises disruption of an AID gene. In some alternatives, the method further comprises introducing production enhancers into the B cell or disruption of candidate loci within the B cell to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody or binding portion thereof is an anti-IL1 monoclonal antibody, anti-TNF antibody, anti-IL33 antibody or anti-C5 antibody or a binding portion of any of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNABs). In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from a group selected from macromolecules, proteins, protein mimetics and peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the isolating is performed by negative selection isolation of hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the method further comprises purifying long lived plasma cells are purified by positive selection against CD138. In some alternatives, purifying comprises selecting the plasma cells by an anti-CD138 antibody for targeted removal of the plasma cells. In some alternatives, the purifying comprises using anti-CD138 beads for plasma cell selection.

In some alternatives, a plasma cell that expresses a molecule, such as a heterologous protein, protein mimetic or a peptide is provided. In some alternatives, the cell is a B cell. In some alternatives, the heterologous protein comprises an enzyme, neutralizing antibody, cytokine, cytokine receptor, complement protein, inhibitory protein, anti-fibrotic molecule, therapeutic antibody, anti-thrombotic molecule, glucose response element and/or monoclonal antibody or binding portion thereof. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and cancer. In some alternatives, the antibody or binding portion thereof is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNABs). In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies. In some alternatives, the protein is an engineered protein, wherein the engineered protein is protective in viral, fungal, parasitic or bacterial infection. In some alternatives, the engineered protein comprises an antibody or binding portion thereof, wherein the antibody or binding portion thereof is specific for an antigen that is expressed on a virus, fungus, parasite or bacteria. In some alternatives, the protein comprises an antibody or a binding portion thereof, wherein the antibody or a binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cells. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from a group of macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the plasma cell further comprises at least one cell surface protein. In some alternatives, the at least one cell surface protein is CD20.

In some alternatives, a composition comprising the plasma cell manufactured by any one of the alternatives provided herein or comprising the cell of any one of the alternatives herein, is provided. In some alternatives, the plasma cell is derived from a B cell. In some alternatives, the plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or a monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the first round of genome editing comprises transfecting single stranded DNA oligonucleotides for homologous recombination into a candidate genetic loci. In some alternatives, the Cas nuclease comprises Cas1, Cas2, Cas3, Cas4, Cas5, Cas6, Cas7, Cas8 or Cas9. In some alternatives, the first round of genome editing comprises transducing the B cell with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody is an anti-I monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity.

In some alternatives, a method of expressing a molecule, such as a macromolecule in a subject in need is provided, wherein the method comprises: providing or administrating a plasma cell manufactured by any one of the alternative methods provided herein, the plasma cell that expresses a molecule of any one of any one of the alternative plasma cells provided herein, or the composition of any one of the alternatives described herein, into the subject in need. In some alternatives, the method further comprises purifying the plasma cells of step d), wherein the purifying is performed after the expanding step. In some alternatives, the purifying comprises positive selecting of cells against CD138. In some alternatives, the selecting the plasma cells is performed by an anti-CD138 antibody for targeted removal of the plasma cells. In some alternatives, the purifying comprises using anti-CD138 beads for plasma cell selection. In some alternatives, the molecule is a macromolecule, such as a protein, protein mimetic or a peptide. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein is an enzyme. In some alternatives, the enzyme comprises a cofactor for enhanced enzymatic activity. In some alternatives, the composition comprises the plasma cell manufactured by any one of the alternatives provided herein or comprises the cell of any one of the alternatives provide herein. In some alternatives, the cell is a B cell. In some alternatives, the B cells comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or monoclonal antibody. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, the protein is the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is an early pro-B cell, late pro-B cell, large pre-B cell, small pre-B cell, immature B cell, T1 B cell, T2 B cell, marginal zone B cell, mature B cell, naïve B cell, plasmablast (short lived) cell, GC B cell, memory B cell, plasmablast cell and/or long lived plasma cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or portion thereof, wherein the antibody or portion thereof is specific for an antigen that is expressed on a virus, fungus, parasite or bacteria. In some alternatives, the protein comprises an antibody or binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cells. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives of the method of expressing a molecule, such as a macromolecule in a subject in need, the administrating is performed by adoptive transfer. In some alternatives of the method of expressing a molecule, such as a macromolecule in a subject in need, the subject has received a stem cell administration or solid organ transplantation. In some alternatives of the method of expressing protein in a subject in need, the subject suffers from influenza, parainfluenza, rhinovirus, respiratory syncytial virus, HIV, or other pathogenic bacteria, virus, fungus or parasite infection. In some alternatives of the method of expressing protein in a subject in need, the subject suffers from an enzyme deficiency. In some alternatives of the method of expressing a molecule, such as a macromolecule in a subject in need, the subject suffers from pulmonary fibrosis. In some alternatives of the method of expressing a molecule, such as a macromolecule in a subject in need, the subject suffers from an autoimmune disorder, immune dysregulation or cancer. In some alternatives, the subject suffers from HIV. In some alternatives of the method of expressing a molecule, such as a macromolecule in a subject in need, the subject suffers from diabetes. In some alternatives of the method of expressing a molecule, such as a macromolecule in a subject in need, the subject suffers from hypercholesterolemia. In some alternatives of the method of expressing a molecule, such as a macromolecule in a subject in need, the subject is also receiving CAR T-cell therapy. In some alternatives, the B cells comprise B cell precursors such as hematopoietic stem cells (HSCs), multipotent progenitor (MPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the method comprises monitoring of the subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a composition comprising the plasma cell manufactured by any one of the alternatives herein and a second B cell, wherein the second B cell secretes a macromolecule for inducing tolerance of a peptide for immunotherapeutic application or for inducing engraftment of the plasma cell manufactured by any one of anyone of the alternatives herein is provided. In some alternatives, the plasma cell is derived from a B cell. In some alternatives, the plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or a monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-I monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the first round of genome editing comprises transfecting single stranded DNA oligonucleotides for homologous recombination into a candidate genetic loci. In some alternatives, the Cas nuclease comprises Cas1, Cas2, Cas3, Cas4, Cas5, Cas6, Cas7, Cas8 or Cas9. In some alternatives, the first round of genome editing comprises transducing the B cell with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the method further comprises purifying the plasma cells or plasma cell precursors by positive selection against CD138. In some alternatives, purifying comprises selecting the plasma cells by an anti-CD138 antibody for targeted removal of the plasma cells. In some alternatives, the purifying comprises using anti-CD138 beads for plasma cell selection.

In some alternatives, a method of treating, ameliorating or inhibiting a disease, such as a cancer, in a subject in need, is provided, the method comprises administering a plasma cell manufactured by any one of the alternative methods herein or the plasma cell that expresses a molecule, such as a macromolecule manufactured by any one of the alternative methods herein, or the composition of any one of the alternative compositions herein into the subject in need. In some alternatives, the composition comprises the plasma cell manufactured by any one of the alternatives provided herein or comprises the cell of any one of the alternatives provided herein. In some alternatives, the cell is a B cell. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or a monoclonal antibody. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof that is specific for a protein or other molecule expressed in autoimmune disorders, autoinflammatory disorders, immune dysregulation and/or cancer. In some alternatives, the antibody is an anti-I monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cell to enable selectable expansion of the B cells in vitro or in vivo. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence of any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody is an anti-I monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives of the method of treating, inhibiting or ameliorating a disease, such as cancer, in a subject in need, the administering is performed by adoptive transfer. In some alternatives of the method of treating, inhibiting, or ameliorating a disease, such as cancer, in a subject in need, the subject has received a stem cell administration or solid organ transplantation. In some alternatives of the method of ameliorating, treating, or inhibiting a disease in a subject in need, the subject suffers from influenza, parainfluenza, rhinovirus, respiratory syncytial virus, HIV, or other pathogenic bacteria, virus, fungus or parasite infection. In some alternatives of the method of ameliorating, treating, or inhibiting a disease in a subject in need, the subject suffers from an enzyme deficiency. In some alternatives of the method of ameliorating, treating, or inhibiting a disease in a subject in need, the subject suffers from pulmonary fibrosis. In some alternatives of the method of ameliorating, treating, or inhibiting a disease in a subject in need, the subject suffers from an autoimmune disorder, immune dysregulation and/or cancer. In some alternatives of the method of ameliorating, treating, or inhibiting a disease in a subject in need, the subject suffers from diabetes. In some alternatives of the method of ameliorating, treating, or inhibiting a disease in a subject in need, the subject suffers from hypercholesterolemia. In some alternatives of the method of ameliorating, treating, or inhibiting a disease in a subject in need, the subject is receiving therapy. In some alternatives of the method of ameliorating, treating, or inhibiting a disease in a subject in need, the subject is selected to receive cancer therapy, diabetes therapy, HIV treatment, or viral therapy. In some alternatives of the method of ameliorating, treating, or inhibiting a disease in a subject in need, the subject is also receiving CAR T-cell therapy. In some alternatives, the B cells comprise B cell precursors such as hematopoietic stem cells (HSCs), multipotent progenitor (MPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of expressing a molecule, such as a macromolecule in a subject of need is provided, wherein the method comprises: (a) isolating B cells; (b) developing the B cells; (c) performing a first round of genome editing of the B cells for molecule expression in absence of viral integration; (d) expanding the B cells; differentiating the B cells, optionally, after step (c) or (d), thereby producing plasma cells that express a protein; and (e) administering the B cells into the subject in need. In some alternatives, the developing of the B cells is performed after the B cells are arrested at a specific phase of development and/or activating the B cells such that the B cells are permissive for recombination without further B cell differentiation. In some alternatives, the method further comprises purifying the plasma cells of step d), wherein the purifying is performed after the expanding step. In some alternatives, the purifying comprises positive selecting of cells against CD138. In some alternatives, the selecting the plasma cells is performed by an anti-CD138 antibody for targeted removal of the plasma cells. In some alternatives, the purifying comprises using anti-CD138 beads for plasma cell selection. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein is an enzyme. In some alternatives, the enzyme comprises a cofactor for enhanced enzymatic activity. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, wherein step (a) performing a second round of genome editing on the B cells to excise a region, further comprises removing IgM positive cells. In some alternatives, the method further comprises, activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/auto-inflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the administering is performed by adoptive transfer. In some alternatives, the subject has received a stem cell by administration or solid organ transplantation. In some alternatives, the subject suffers from influenza, parainfluenza, rhinovirus, respiratory syncytial virus, HIV, or other pathogenic bacteria, virus, fungus or parasite infection. In some alternatives, the subject suffers from an enzyme deficiency. In some alternatives, the subject suffers from pulmonary fibrosis. In some alternatives, the subject suffers from an autoimmune disorder, immune dysregulation and/or cancer. In some alternatives, the subject suffers from diabetes. In some alternatives, the subject suffers from hypercholesterolemia. In some alternatives, the subject is receiving a conventional therapy for the disease, malady or condition. In some alternatives, the subject is selected to receive cancer therapy, diabetes therapy, HIV treatment, or viral therapy. In some alternatives, the subject is also receiving CAR T-cell therapy. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of ameliorating a disease in a subject in need is provided, the method comprising: administering a plasma cell manufactured by any one of the alternatives herein or the plasma cell that expresses a molecule, such as a macromolecule manufactured by any one of the alternatives herein, or the composition of the alternatives herein into the subject in need. In some alternatives, the administering is performed by adoptive transfer. In some alternatives, the subject has received a stem cell administration or solid organ transplantation. In some alternatives, the subject suffers from influenza, parainfluenza, rhinovirus, respiratory syncytial virus, HIV, or other pathogenic bacteria, virus, fungus or parasite infection. In some alternatives, the composition comprises the plasma cell manufactured by any one of the alternatives provided herein or comprises the cell of any one of the alternatives provided herein. In some alternatives, the plasma cell is derived from a B cell. In some alternatives, the plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or a monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the first round of genome editing comprises transfecting single stranded DNA oligonucleotides for homologous recombination into a candidate genetic loci. In some alternatives, the Cas nuclease comprises Cas1, Cas2, Cas3, Cas4, Cas5, Cas6, Cas7, Cas8 or Cas9. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence of any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGBIAI. In some alternatives, the therapeutic antibodies or binding portions thereof are specific for a protein expressed in autoimmune disorders, autoinflammatory disorders, immune dysregulation and/or cancer. In some alternatives, the antibody is an anti-I1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the B cells comprise B cell precursors such as hematopoietic stem cells (HSCs), multipotent progenitor (VPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of enhancing immune tolerance to protein therapeutics in a subject in need is provided, the method comprising: administering a plasma cell manufactured by any one of the alternatives herein or the plasma cell that a macromolecule manufactured by any one of the alternatives herein, or the composition of the alternatives herein into the subject in need. In some alternatives, the macromolecule is a protein for inducing tolerance of a protein or peptide for therapy or induces or promotes engraftment and/or survival. In some alternatives, the macromolecule is a protein. The protein may be BAFF, APRIL, IFN-alpha, IL-10 and/or IL-6. In some alternatives the protein is a viral vector capsid protein. In some alternatives, the method further comprises purifying the plasma cells of step D), wherein the purifying is performed after the differentiating step. In some alternatives, the purifying comprises positive selecting of cells against CD138. In some alternatives, the selecting the plasma cells is performed by an anti-CD138 antibody for targeted removal of the plasma cells. In some alternatives, the purifying comprises using anti-CD138 beads for plasma cell selection. In some alternatives, the viral vector capsid protein comprises one or more of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) or AAV9 (VP1, VP2 and VP3). In some alternatives, the composition comprises the plasma cell manufactured by any one of the alternatives provided herein or comprises the cell of any one of the alternatives provided herein. In some alternatives, the plasma cell is derived from a B cell. In some alternatives, the plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or a monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cells. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cells. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the first round of genome editing comprises transfecting single stranded DNA oligonucleotides for homologous recombination into a candidate genetic loci. In some alternatives, the Cas nuclease comprises Cas1, Cas2, Cas3, Cas4, Cas5, Cas6, Cas7, Cas8 or Cas9. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies or binding portions thereof are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of enhancing immune tolerance to a protein therapeutic in a subject in need is provided. The method comprises administering a B cell manufactured by any one of the alternatives provided herein or the B cell that expresses a molecule, such as a macromolecule manufactured by any one of the alternatives provided herein, or the composition of any one of the alternatives provided herein into the subject in need. In some alternatives, the subject suffers from Hemophilia A, Hemophilia B, Hereditary TPP, lysosomal acid deficiency, hereditary angioedema, alphal anti-trypsin deficiency, Fabry disease or hypophosphatasia. B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixture or combination thereof. In some alternatives, the composition comprises the plasma cell manufactured by any one of the alternatives provided herein or comprises the cell of any one of the alternatives provided herein. In some alternatives, the plasma cell is derived from a B cell. In some alternatives, the plasma cell expresses a molecule, such as a heterologous protein, protein mimetic or a peptide. In some alternatives, the cell is a B cell precursor. In some alternatives, the B cell precursor comprises hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the protein comprises an enzyme, neutralizing antibody, cytokine, cytokine receptor, complement protein, inhibitory protein, anti-fibrotic molecule, therapeutic antibody, anti-thrombotic molecule, glucose response element or a monoclonal antibody. In some alternatives, the protein is Factor VIII, Factor IX, SERPING1 or SERPINA1. In some alternatives, the enzyme is ADAMTS13, LIPA, GLA, or ALPL. In some alternatives, the enzyme is ADAMTS13, LIPA, GLA, or ALPL. In some alternatives, the protein is a receptor antagonist for treatment or amelioration of periodic fever or autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGB1A1. In some alternatives, the therapeutic antibodies are specific for a protein expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody. In some alternatives, the protein comprises broadly neutralizing HIV-1 antibodies (bNAbs) or a binding portion thereof. In some alternatives, the protein comprises a prodrug or a proenzyme. In some alternatives, the plasma cell is a long lived plasma cell. In some alternatives, the plasma cell is manufactured by any one of the alternative methods provided herein. In some alternatives, the plasma cell is manufactured by a method of making plasma cells or plasma cell precursors that express a molecule, such as a macromolecule, wherein the method comprises (a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells that express a protein. In some alternatives, the developing of the B cells is performed after the B cells are arrested at a specific phase of development and/or activating the B cells such that the B cells are permissive for recombination without further B cell differentiation. In some alternatives, the macromolecule is a protein, protein mimetic or a peptide. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the nuclease or synthetic guide RNAs targeting clustered regularly interspersed short palindromic repeat DNA (CRISPR) coupled to a Cas nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, the therapeutic antibody or a binding portion thereof is specific for a protein or other molecule expressed in autoimmune disorders, autoinflammatory disorders, immune dysregulation and/or cancer. In some alternatives, the antibody is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the protein is an engineered protein, wherein the engineered protein is protective in viral, fungal, parasitic or bacterial infection. In some alternatives, the engineered protein comprises an antibody or a binding portion thereof, wherein the antibody or a binding portion thereof is specific for an antigen that is expressed on a virus, fungus, parasite or bacteria. In some alternatives, the protein comprises an antibody or a binding portion thereof, wherein the antibody or binding portion thereof is specific for an antigen that is expressed in a viral, fungal, parasitic or bacterial infection. In some alternatives, the plasma cell is manufactured by a method of making a long lived plasma cell, wherein the method comprises: isolating B cells; activating the B cells; a first round of genome editing of the B cells for protein expression in the absence of viral integration; expanding the B cells; and differentiating the B cells. In some alternatives, the B cells are subject derived or are allogeneic peripheral blood mononuclear cells. In some alternatives, the B cells are blood-derived human B cells. In some alternatives, the first round of genome editing is performed by an RNA or protein based transfection. In some alternatives, the first round of genome editing comprises delivering a nuclease, wherein the nuclease targets at least one genetic locus in the B cell. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives, the first round of genome editing comprises transfecting single stranded DNA oligonucleotides for homologous recombination into a candidate genetic loci. In some alternatives, the Cas nuclease comprises Cas1, Cas2, Cas3, Cas4, Cas5, Cas6, Cas7, Cas8 or Cas9. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever/autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGBIAI. In some alternatives, the therapeutic antibodies or binding portions thereof are specific for a protein expressed in autoimmune disorders, autoinflammatory disorders, immune dysregulation and/or cancer. In some alternatives, the antibody is an anti-I monoclonal antibody, anti-TNF antibody, anti-IL33 antibody or anti-C5 antibody. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cells, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of inducing tolerance of a macromolecule secreted by a B cell in a subject in need in vivo is provided, the method comprising: A) isolating B cells; B) performing a first round of genome editing of the B cells for protein expression in absence of viral integration, wherein the performing is under conditions of rapid B cell expansion; C) expanding the B cells; D) differentiating the B cells, optionally, after step (c) or (d), thereby producing activated B cells or naïve activated B cells that express the molecule; and E) providing or administering the B cells that expresses the macromolecule into a subject in need. In some alternatives, the macromolecule, is a protein, protein mimetic or peptide. In some alternatives, the macromolecule comprises natural amino acids and/or unnatural amino acids. In some alternatives, the protein is an enzyme. In some alternatives, the method further comprises activating the B cells prior to gene editing, where inactivating is performed using at least one stimulant. In some alternatives, the at least one stimulant is a CD40 ligand, CpG, IL2, IL10 and/or IL15. In some alternatives, cells are activated with 100 ng/ml of recombinant human MEGACD40L®), 1 μg/ml of CpG oligodeoxynucleotide 2006, 50 ng/ml of IL2, 50 ng/ml of IL10 and 10 ng/ml of IL15 for two days. In some alternatives, the CD40 ligand is artificially oligermized. In some alternatives, the CD40 ligand is oligermerized with two linked CD40L trimers. In some alternatives, the cells are cultured for at least 13 days with the at least one stimulant. In some alternatives, the cells are a naïve B cell phenotype, activated B cell or activated edited B cell after differentiation. In some alternatives, the cells are CD27−CD138−CD38low/—CD19highIgM+ IgD+ after differentiation. In some alternatives, the providing or administering is performed by adoptive transfer. In some alternatives, the subject has received a stem cell administration or solid organ transplantation. In some alternatives, the subject suffers from influenza, parainfluenza, rhinovirus, respiratory syncytial virus, HIV, or other pathogenic bacteria, virus, fungus or a parasite infection. In some alternatives, the subject suffers from an enzyme deficiency. In some alternatives, the subject suffers from pulmonary fibrosis. In some alternatives, the subject suffers from an autoimmune disorder, immune dysregulation or cancer. In some alternatives, the subject suffers from diabetes. In some alternatives, the subject suffers from hypercholesterolemia. In some alternatives, the subject is also receiving CAR T-cell therapy. In some alternatives, the subject suffers from HIV. In some alternatives, the subject suffers from Hemophilia A, Hemophilia B, Hereditary TPP, lysosomal acid deficiency, hereditary angioedema, alphal anti-trypsin deficiency, Fabry disease or hypophosphatasia. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the first round of genome editing is performed by an RNA and protein based transfection. In some alternatives, the first round of genome editing comprises delivering a nuclease, wherein the nuclease targets at least one genetic loci in the B cell. In some alternatives, the first round of genome editing comprises delivering a nuclease, wherein the nuclease targets at least one genetic loci in the B cells and co-delivering the nuclease with a single stranded nucleic acid donor template. In some alternatives, the nuclease is a synthetic guide RNA targeting clustered regularly interspersed short palindromic repeat DNA (CRISPR) coupled to a Cas nuclease. B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of inducing tolerance of a peptide useful for immunotherapeutic application, secreted by a B cell in a subject in need in vivo and/or increasing engraftment of the B cell in a subject in need, the method comprising: A) isolating a first population of B cells; B) performing a first round of genome editing of the first population of B cells for protein or peptide expression in absence of viral integration, wherein the performing is under conditions of rapid B cell expansion; C) expanding the first population of B cells; D) differentiating the first population of B cells, optionally, after step (c) or (d), thereby producing an activated first population of B cells or a naïve first population of activated B cells that express the molecule; E) providing or administering the first population of B cells that expresses the peptide into a subject in need; and F) administering a second population of B cells that expresses a macromolecule, such as BAFF, APRIL, IFN-alpha, IL-10 or TL-6. In some alternatives, the immunotherapeutic application is treatment or amelioration of cancer. In some alternatives, the cancer comprises a solid tumor. In some alternatives, the peptide is an anti-PD1 antibody or a PDL-1 ligand. In some alternatives, the second population of B cells express BAFF, APRTL, IFN-alpha, IL-10 or IL-6. In some alternatives, the second population of B cells express a second peptide that is expressed and presented as a MHC class II molecule on a surface of the B cells. B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a method of inducing tolerance of a peptide for immunotherapeutic application, secreted by a B cell in a subject in need in vivo and/or increasing engraftment or survival of the B cells in a subject in need, the method comprising: A) isolating a first population of B cells; B) performing a first round of genome editing of the first population of B cells for protein or peptide expression in absence of viral integration, wherein the performing is under conditions of rapid B cell expansion; C) expanding the first population of B cells; D) differentiating the first population of B cells, optionally, after step (c) or (d), thereby producing activated B cells or naïve activated B cells that express the protein or peptide; E) providing or administering the first population of B cells that express the protein or peptide into a subject in need F) providing or administering a second population of B cells that express a macromolecule, wherein the second population of B cells allows tolerance of the protein or peptide and/or promotes engraftment. In some alternatives, the first population of B cells and the second population of B cells are co-administered. In some alternatives, the macromolecule comprises BAFF, APRTL, IL-10, IFN-alpha or IL-6. In some alternatives, the protein comprises an enzyme, neutralizing antibody or a binding portion thereof, cytokine, cytokine receptor, complement protein, inhibitory protein, anti-fibrotic molecule, therapeutic antibody or a binding portion thereof, anti-thrombotic molecule, glucose response element or monoclonal antibody or a binding portion thereof. In some alternatives the protein is a viral vector capsid protein. In some alternatives, the viral vector capsid protein comprises one or more of one of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) or AAV9 (VP1, VP2 and VP3). In some alternatives, the protein is a receptor antagonist for the treatment or inhibition of periodic fever/autoinflammatory syndromes, complement inhibitory proteins (including Factor H, Factor I) for the treatment or inhibition of atypical hemalytic uremic syndrome/membranoproliferative glomerulonephritis or a C1 inhibitor for hereditary angioedema. In some alternatives, the protein is an anti-fibrotic molecule, wherein the anti-fibrotic molecule is SCGB1A1. In some alternatives, the protein comprises therapeutic antibodies or a binding portion thereof are specific for a protein expressed in autoimmune disorders, autoinflammatory disorders, immune dysregulation and/or cancer. In some alternatives, the antibody or a binding portion thereof is an anti-IL1 monoclonal antibody, anti-TNF antibody, anti-IL33 antibody or anti-C5 antibody or a binding portion of any one or more of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNAbs) or a binding portion thereof. B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof. In some alternatives, the method comprises monitoring of subject following plasma cell transplantation. In some alternatives, the subject is determined to no longer need treatment or therapy and the method further comprises administering rituxan® or an anti-CD20 antibody or binding portion thereof for targeted removal of the plasma cells.

In some alternatives, a composition is provided, wherein the composition comprises a first population of B cells manufactured by any one of the alternatives herein and a second population of B cells, wherein the second population of B cells secretes a macromolecule for inducing tolerance of a peptide for immunotherapeutic application or for inducing engraftment of the plasma cell manufactured by any one of the alternatives herein. In some alternatives, the macromolecule expressed by the second population of B cells comprises BAFF, IFN-alpha, APRIL, IL-10 or IL-6. In some alternatives the macromolecule is a viral vector capsid protein. In some alternatives, the viral vector capsid protein comprises one or more of one of the following serotypes: AAV1 (VP1, VP2 and VP3), AAV2 (VP1, VP2 and VP3), AAV4 (VP1, VP2 and VP3), AAV5 (VP1, VP2 and VP3), AAV6 (VP1, VP2 and VP3), AAV1 (VP1, VP2 and VP3), AAV8 (VP1, VP2 and VP3), AAV1 (VP1, VP2 and VP3) or AAV9 (VP1, VP2 and VP3). B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long- or short-lived plasma cells and/or any mixtures or combinations thereof. In some alternatives, the method of making plasma cells or plasma cell precursors that expresses a molecule, such as a macromolecule, protein, or peptide comprises(a) isolating B cells; (b) developing the B cells; (c) performing a first round of genome editing of the B cells for protein expression in absence of viral integration; (d) expanding the B cells; and (e) differentiating the B cells, optionally, after step (c) or (d), thereby producing plasma cells or plasma cell precursors that express the molecule. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the developing of the B cells is performed after the B cells are arrested at a specific phase of development and/or activating the B cells such that the B cells are permissive for recombination without further B cell differentiation. In some alternatives, the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell. In some alternatives, performing the first round of genome editing of the B cells for protein expression is performed in the absence of viral integration. In some alternatives, performing the first round of genome editing of the B cells for protein expression is performed by introduction of a single stranded nucleic acid. In some alternatives, the B cells are subject derived or are allogeneic peripheral blood mononuclear cells. In some alternatives, the B cells are blood-derived human B cells. In some alternatives, the first round of genome editing is performed by an RNA and protein based transfection. In some alternatives, the first round of genome editing comprises delivering a nuclease, wherein the nuclease targets at least one genetic loci in the B cells. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a CAS nuclease. In some alternatives, the nuclease or synthetic guide RNAs targeting clustered regularly interspersed short palindromic repeat DNA (CRISPR) coupled to a CAS nuclease are delivered via translatable RNA or recombinant protein. In some alternatives, the Cas nuclease comprises Cas1, Cas2, Cas3, Cas4, Cas5, Cas6, Cas7, Cas8 or Cas9. In some alternatives, the first round of genome editing comprises transfecting with single stranded DNA oligonucleotides for homologous recombination into a candidate genetic loci. In some alternatives, the single stranded DNA oligonucleotides are unmodified. In some alternatives, the single stranded DNA oligonucleotides are modified with 5′ or 3′ phosphorothioate linkages as repair templates. In some alternatives, the first round of genome editing comprises transducing the B cells with a recombinant adeno-associated virus vector to serve as a donor template for homologous recombination into a candidate genetic loci. In some alternatives, the recombinant adeno-associated virus vector is single-stranded, double stranded or self-complementary. In some alternatives, the method further comprises applying methods to prevent somatic hypermutation of an antibody locus in the B cells. In some alternatives, the method to prevent somatic hypermutation of an antibody locus in the B cells comprises disruption of an AID gene. In some alternatives, the method further comprises selectively increasing a proportion of gene edited B cells. In some alternatives, the increasing the proportion of gene edited B cells comprises the steps of: (a) performing a second round of genome editing on the B cells to excise a region; (b) performing a third round of genome editing on the B cells, wherein the third round of genome editing results in expression of drug activatable growth enhancers; (c) RNA transfecting into the B cells short lived drug activatable growth enhancers; and (d) inserting genetic modifications that artificially induce non-transformative expansion of gene edited B cells. In some alternatives, the first round of genome editing further comprises techniques for homology-directed repair. In some alternatives, the second round of genome editing results in the excision of the IgM constant region. In some alternatives, the second round of genome editing results in the B cells mimicking a class-switch to IgG1 positive cells. In some alternatives, step (a) of the step increasing the proportion of gene edited B cells further comprises removing IgM positive cells. In some alternatives, the method further comprises activating the IgG1 positive cells and expanding the IgG1 positive cells, wherein the expanding is performed by exogenous antigens that specifically bind the inserted surface-expressed IgG1. In some alternatives, the IgM positive cells are removed by negative selection. In some alternatives, the expanding of the IgG1 positive cells is performed by exogenous antigens that specifically bind the inserted surface-expressed IgG1. In some alternatives, the drug activatable growth enhancers expressed after the third round of genome editing are rapamycin-inducible FKBP11 dimers. In some alternatives, the activatable growth enhancers that are RNA transfected into the B cells of step (c) are rapamycin-inducible FKBP11 dimers. In some alternatives, the inserting genetic modifications that artificially induce triggerable, non-transformative expansion of edited cells results in disruption of a HPRT locus and negative selection of non-edited cells. In some alternatives, the negative selecting of non-edited cells is performed by 6-thioguanine. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the B-cells for homologous recombination of the single stranded DNA oligonucleotides or recombinant adeno-associated virus into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or any other length defined by a range that is set forth by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by isolation of naïve or memory B cells. In some alternatives, the isolating is performed from isolation from healthy donor PBMCs or cell apheresis collections. In some alternatives, the differentiating step is performed in a three-step culture system comprising an activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element and/or a synthetic engineered protein. In some alternatives, the protein is IFN-alpha, IL-6, Factor VIII, Factor IX, SERPING1 or SERPINA1. In some alternatives, the enzyme is ADAMTS13, LIPA, GLA, or ALPL. In some alternatives, the protein is a receptor antagonist that is used for the treatment or amelioration of periodic fever/autoinflammatory syndromes or complement inhibitory proteins. In some alternatives, the compliment inhibitory protein is Factor H, Factor I or a C1 inhibitor. In some alternatives, the protein is an anti-fibrotic molecule, wherein the anti-fibrotic molecule is SCGB1A1. In some alternatives, a therapeutic antibody or binding portion thereof is used e.g., an antibody or binding portion thereof that is specific for a protein or other molecule expressed in an autoimmune disorder, autoinflammatory disorder, immune dysregulation and/or cancer. In some alternatives, the antibody or binding portion thereof is an anti-IL1 monoclonal antibody, anti-TNF antibody, anti-IL33 antibody or anti-C5 antibody or a binding portion thereof. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies. In some alternatives, the protein is an engineered protein, wherein the engineered protein is protective in viral, fungal, parasitic and/or bacterial infection. In some alternatives, the engineered protein comprises an antibody or a binding portion thereof, wherein the antibody or binding portion thereof is specific for an antigen that is expressed on a virus, fungus, parasite or bacteria. In some alternatives, the protein comprises an antibody or binding portion thereof, wherein the antibody or binding portion thereof is specific for an antigen that is expressed in a viral, fungal, parasitic or bacterial infection. In some alternatives, the B cells comprise B cell precursors such as hematopoietic stem cells (HSCs), multipotent progenitor (MPP) cells, lymphoid progenitor (CLP) cells, naïve B cells, GC B cell, plasmablast, early pro-B cells, late pro-B cells, large pre-B cells, small pre-B cells, immature B cells, T1 B cells, T2 B cells, marginal zone B cells, mature B cells and memory B cells. In some alternatives, the molecule is a macromolecule, protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein comprises a cofactor for enhanced enzymatic activity. In some alternatives, the method of making a long lived plasma cell comprises isolating B cells; activating the B cells; a first round of genome editing of the B cells for molecule expression, such as a protein, protein mimetic or a peptide in the absence of viral integration; expanding the B cells; and differentiating the B cells. In some alternatives, the molecule is a macromolecule, such as a protein, protein mimetic or peptide. In some alternatives, the macromolecule is a prodrug. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acylated. In some alternatives, the protein is an enzyme. In some alternatives, the enzyme comprises a cofactor for enhanced enzymatic activity. In some alternatives, the B cells in step (a) comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the B cells are subject derived or are allogeneic peripheral blood mononuclear cells. In some alternatives, the B cells are blood-derived human B cells. In some alternatives, the B cells from the isolating step comprise B cell precursors such as hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the first round of genome editing is performed by an RNA or protein based transfection. In some alternatives, the first round of genome editing comprises delivering a nuclease, wherein the nuclease targets at least one genetic locus in the B cells. In some alternatives, the nuclease is a zinc finger nuclease, transcription activator-like effector nuclease (TALEN), homing endonuclease (HEs), combined TALEN-HE protein (megaTALs) or clustered regularly interspersed short palindromic repeat DNA (CRISPR) coupled to a Cas nuclease. In some alternatives the Cas nuclease comprises Cas1, Cas2, Cas3, Cas4, Cas5, Cas6, Cas7, Cas8 or Cas9. In some alternatives, the first round of genome editing comprises transfecting single stranded DNA oligonucleotides for homologous recombination into a candidate genetic loci. In some alternatives, the first round of genome editing comprises transducing the B cells with a recombinant adeno-associated virus vector for homologous recombination into a candidate genetic loci. In some alternatives, the method further comprises applying methods to prevent somatic hypermutation of an antibody locus and other target loci in the B cells. In some alternatives, the method to prevent somatic hypermutation of an antibody locus in the B cells comprises disruption of an AID gene. In some alternatives, the method further comprises introducing production enhancers into the B cells or disruption of candidate loci within the B cells to enable selectable expansion of the B cells in vitro or in vivo. In some alternatives, the method further comprises introducing a nucleic acid, wherein the nucleic acid encodes at least one cell surface protein. In some alternatives, the at least one cell surface protein is CD20. In some alternatives, the first round of genome editing further comprises cycling the cells for homologous recombination of the single stranded DNA oligonucleotides or the recombinant adeno-associated virus vector into the candidate genetic loci. In some alternatives, the single stranded DNA oligonucleotides or recombinant adeno-associated virus comprises a length of 0.2 kb, 0.5 kb, 1 kb, 2 kb, 3 kb, 4 kb or 5 kb or a length within a range defined by any two of the aforementioned values. In some alternatives, the isolating is performed by isolation of early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells. In some alternatives, the isolating is performed by negative selection isolation of naïve or memory B cells. In some alternatives, the naïve or memory B cells are from healthy donor PBMCs or cells collected by apheresis. In some alternatives, the differentiating step is performed in a three-step culture system comprising an activation and proliferation step, a plasmablast differentiation step and a plasma cell differentiation step. In some alternatives, the activation and proliferation step is performed in the presence any combination of MCD40L, CpG, IL-2, IL-10 and/or IL-15. In some alternatives, the plasmablast differentiation step is performed in the presence of any combination of IL-2, IL-6, IL-10 and/or IL-15. In some alternatives, the plasma cell differentiation step is performed in the presence of any combination of IL-6, IL-15, APRIL and/or IFNα. In some alternatives, the single stranded DNA oligonucleotides or the recombinant adenovirus comprises a sequence encoding the protein. In some alternatives, the protein comprises an enzyme, monoclonal antibody, neutralizing antibodies, therapeutic antibodies, cytokine, cytokine receptor, complement protein, inhibitory protein, anti-fibrotic molecule, anti-thrombotic molecule, coagulation factor, glucose response element or a synthetic engineered protein. In some alternatives, the protein is IFN-alpha, Factor VIII, Factor IX, SERPING1 or SERPINA1. In some alternatives, the enzyme is ADAMTS13, LIPA, GLA, or ALPL. In some alternatives, the protein is a receptor antagonist for treatment or amelioration of periodic fever or autoinflammatory syndromes, complement inhibitory proteins (including Factor H, Factor I) for treatment or amelioration of atypical hemalytic uremic syndrome/membranoproliferative glomerulonephritis or a C1 inhibitor for hereditary angiodema. In some alternatives, the protein is an anti-fibrotic molecule, wherein the anti-fibrotic molecule is SCGBIAI. In some alternatives, the therapeutic antibodies or binding portions thereof are specific for a protein expressed in autoimmune disorders, autoinflammatory disorders, immune dysregulation and/or cancer. In some alternatives, the antibody or binding portion thereof is an anti-IL1 monoclonal antibody, anti-TNF antibody, anti-IL33 antibody or anti-C5 antibody or a binding portion of any of these antibodies. In some alternatives, the protein is an anti-thrombotic molecule, wherein the anti-thrombotic molecule is APLN. In some alternatives, the protein comprises an anti-PCSK9 inhibitory antibody or a binding portion thereof. In some alternatives, the protein comprises broadly neutralizing HIV-1 antibodies (bNABs). In some alternatives, the protein comprises a prodrug or a proenzyme. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the molecule is selected from macromolecules, proteins, protein mimetics and/or peptides. In some alternatives, the macromolecule, protein, protein mimetic, or peptide comprises natural amino acids and/or unnatural amino acids. In some alternatives, the molecule comprises a carbohydrate or lipid moiety. In some alternatives, the molecule comprises a cofactor. In some alternatives, the protein comprises a hydrophobic group for membrane localization. In some alternatives, the protein is acetylated. In some alternatives, the protein comprises a cofactor for enhanced enzymatic activity. In some alternatives, the isolating is performed by negative selection isolation of hematopoietic stem cells, early pro-B cells, late pro-B cells, large pre-B cells, small pre-B cells, immature B cells, T1 B cells, T2 B cells, marginal zone B cells, mature B cells, naïve B cells, plasmablast (short lived) cells, GC B cells, memory B cells, plasmablast cells and/or long lived plasma cells.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an alternative of the first through fifteenth aspects is applicable to all aspects and embodiments identified herein. Moreover, any of the features of an alternative of the first through fifteenth aspects is independently combinable, partly or wholly with other embodiments described herein in any way, e.g., one, two, or three or more alternative may be combinable in whole or in part. Further, any of the features of an alternative of the first through fifteenth aspects may be made optional to other aspects or alternatives.

### REFERENCES

1. Radbruch, A, Muehlinghaus, G, Luger, E O, Inamine, A, Smith, KG, Dorner, T, et al. (2006). Competence and competition: the challenge of becoming a long-lived plasma cell. Nature reviews Immunology 6: 741-750.
2. Slifka, MK, Antia, R, Whitmire, J K, and Ahmed, R (1998). Humoral immunity due to long-lived plasma cells. Immunity 8: 363-372.
3. Kim, EK, Seo, HS, Chae, MJ, Jeon, IS, Song, BY, Park, Y J, et al. (2014). Enhanced antitumor immunotherapeutic effect of B-cell-based vaccine transduced with modified adenoviral vector containing type 35 fiber structures. Gene therapy 21: 106-114.
4. Hellebrand, E, Mautner, J, Reisbach, G, Nimmerjahn, F, Hallek, M, Mocikat, R, et al. (2006). Epstein-Barr virus vector-mediated gene transfer into human B cells: potential for antitumor vaccination. Gene therapy 13: 150-162.
5. Frecha, C, Costa, C, Levy, C, Negre, D, Russell, SJ, Maisner, A, et al. (2009). Efficient and stable transduction of resting B lymphocytes and primary chronic lymphocyte leukemia cells using measles virus gp displaying lentiviral vectors. Blood 114: 3173-3180.
6. Serafini, M, Naldini, L, and Introna, M (2004). Molecular evidence of inefficient transduction of proliferating human B lymphocytes by VSV-pseudotyped HIV-1-derived lentivectors. Virology 325: 413-424.
7. Levy, C, Fusil, F, Amirache, F, Costa, C, Girard-Gagnepain, A, Negre, D, et al. (2016). Baboon envelope pseudotyped lentiviral vectors efficiently transduce human B cells and allow active factor IX B cell secretion in vivo in NOD/SCIDgammac−/− mice. Journal of thrombosis and haemostasis: JTH 14: 2478-2492.
8. Levy, C, Amirache, F, Costa, C, Frecha, C, Muller, CP, Kweder, H, et al. (2012). Lentiviral vectors displaying modified measles virus gp overcome pre-existing immunity in in vivo-like transduction of human T and B cells. Molecular therapy: the journal of the American Society of Gene Therapy 20: 1699-1712.
9. Mock, U, Thiele, R, Uhde, A, Fehse, B, and Horn, S (2012). Efficient lentiviral transduction and transgene expression in primary human B cells. Human gene therapy methods 23: 408-415.
10. Hale, M, Mesojednik, T, Romano Ibarra, GS, Sahni, J, Bernard, A, Sommer, K, et al. (2017). Engineering HIV-Resistant, Anti-HIV Chimeric Antigen Receptor T Cells. Molecular therapy: the journal of the American Society of Gene Therapy 25: 570-579.
11. Dever, DP, Bak, RO, Reinisch, A, Camarena, J, Washington, G, Nicolas, C E, et al. (2016). CRISPR/Cas9 beta-globin gene targeting in human haematopoietic stem cells. Nature 539: 384-389.
12. Sather, BD, Romano Ibarra, GS, Sommer, K, Curinga, G, Hale, M, Khan, I F, et al. (2015). Efficient modification of CCR5 in primary human hematopoietic cells using a megaTAL nuclease and AAV donor template. Science translational medicine 7: 307ra156.
13. Mali, P, Yang, L, Esvelt, KM, Aach, J, Guell, M, DiCarlo, J E, et al. (2013). RNA-guided human genome engineering via Cas9. Science 339: 823-826.
14. Cong, L, Ran, FA, Cox, D, Lin, S, Barretto, R, Habib, N, et al. (2013). Multiplex genome engineering using CRISPR/Cas systems. Science 339: 819-823.
15. Chu, VT, Graf, R, Wirtz, T, Weber, T, Favret, J, Li, X, et al. (2016). Efficient CRISPR-mediated mutagenesis in primary immune cells using CrispRGold and a C57BL/6 Cas9 transgenic mouse line. Proceedings of the National Academy of Sciences of the United States of America 113: 12514-12519.
16. Pogson, M, Parola, C, Kelton, WJ, Heuberger, P, and Reddy, ST (2016). Immunogenomic engineering of a plug-and-(dis)play hybridoma platform. Nature communications 7: 12535.
17. Cheong, TC, Compagno, M, and Chiarle, R (2016). Editing of mouse and human immunoglobulin genes by CRISPR-Cas9 system. Nature communications 7: 10934.
18. Gwiazda, KS, Grier, AE, Sahni, J, Burleigh, SM, Martin, U, Yang, J G, et al. (2016). High Efficiency CRISPR/Cas9-mediated Gene Editing in Primary Human T-cells Using Mutant Adenoviral E4orf6/Elb55k “Helper” Proteins. Molecular therapy: the journal of the American Society of Gene Therapy 24: 1570-1580.
19. Lin, S, Staahl, BT, Alla, R K, and Doudna, JA (2014). Enhanced homology-directed human genome engineering by controlled timing of CRISPR/Cas9 delivery. eLife 3: e04766.
20. Heyer, WD, Ehmsen, K T, and Liu, J (2010). Regulation of homologous recombination in eukaryotes. Annual review of genetics 44: 113-139.
21. Jiang, W, Lederman, MM, Harding, CV, Rodriguez, B, Mohner, R J, and Sieg, SF (2007). TLR9 stimulation drives naïve B cells to proliferate and to attain enhanced antigen presenting function. European journal of immunology 37: 2205-2213.
22. Armitage, RJ, Macduff, BM, Eisenman, J, Paxton, R, and Grabstein, KH (1995). IL-15 has stimulatory activity for the induction of B cell proliferation and differentiation. Journal of immunology 154: 483-490.
23. Armitage, RJ, Macduff, BM, Spriggs, M K, and Fanslow, WC (1993). Human B cell proliferation and Ig secretion induced by recombinant CD40 ligand are modulated by soluble cytokines. Journal of immunology 150: 3671-3680.
24. Rottman, JB, Ganley, KP, Williams, K, Wu, L, Mackay, C R, and Ringler, DJ (1997). Cellular localization of the chemokine receptor CCR5. Correlation to cellular targets of HIV-1 infection. The American journal of pathology 151: 1341-1351.
25. Ran, FA, Hsu, PD, Wright, J, Agarwala, V, Scott, D A, and Zhang, F (2013). Genome engineering using the CRISPR-Cas9 system. Nature protocols 8: 2281-2308.
26. Richardson, CD, Ray, GJ, DeWitt, MA, Curie, G L, and Corn, JE (2016). Enhancing homology-directed genome editing by catalytically active and inactive CRISPR-Cas9 using asymmetric donor DNA. Nature biotechnology 34: 339-344.
27. Jourdan, M, Caraux, A, De Vos, J, Fiol, G, Larroque, M, Cognot, C, et al. (2009). An in vitro model of differentiation of memory B cells into plasmablasts and plasma cells including detailed phenotypic and molecular characterization. Blood 114: 5173-5181.
28. Ochiai, K, Maienschein-Cline, M, Simonetti, G, Chen, J, Rosenthal, R, Brink, R, et al. (2013). Transcriptional regulation of germinal center B and plasma cell fates by dynamical control of IRF4. Immunity 38: 918-929.
29. Shaffer, A L, Lin, KI, Kuo, TC, Yu, X, Hurt, E M, Rosenwald, A, et al. (2002). Blimp-1 orchestrates plasma cell differentiation by extinguishing the mature B cell gene expression program. Immunity 17: 51-62.
30. Muto, A, Ochiai, K, Kimura, Y, Itoh-Nakadai, A, Calame, KL, Ikebe, D, et al. (2010). Bach2 represses plasma cell gene regulatory network in B cells to promote antibody class switch. The EMBO journal 29: 4048-4061.
31. Nera, KP, Kohonen, P, Narvi, E, Peippo, A, Mustonen, L, Terho, P, et al. (2006). Loss of Pax5 promotes plasma cell differentiation. Immunity 24: 283-293.
32. Muto, A, Tashiro, S, Nakajima, O, Hoshino, H, Takahashi, S, Sakoda, E, et al. (2004). The transcriptional programme of antibody class switching involves the repressor Bach2. Nature 429: 566-571.
33. Kozmik, Z, Wang, S, Dorfler, P, Adams, B, and Busslinger, M (1992). The promoter of the CD19 gene is a target for the B-cell-specific transcription factor BSAP. Molecular and cellular biology 12: 2662-2672.
34. Khan, IF, Hirata, R K, and Russell, DW (2011). AAV-mediated gene targeting methods for human cells. Nature protocols 6: 482-501.
35. Hirata, R, Chamberlain, J, Dong, R, and Russell, DW (2002). Targeted transgene insertion into human chromosomes by adeno-associated virus vectors. Nature biotechnology 20: 735-738.
36. Russell, D W, and Hirata, RK (1998). Human gene targeting by viral vectors. Nature genetics 18: 325-330.
37. Challita, P M, Skelton, D, el-Khoueiry, A, Yu, XJ, Weinberg, K, and Kohn, DB (1995). Multiple modifications in cis elements of the long terminal repeat of retroviral vectors lead to increased expression and decreased DNA methylation in embryonic carcinoma cells. Journal of virology 69: 748-755.
38. Liu, R, Paxton, WA, Choe, S, Ceradini, D, Martin, SR, Horuk, R, et al. (1996). Homozygous defect in HIV-1 coreceptor accounts for resistance of some multiply-exposed individuals to HIV-1 infection. Cell 86: 367-377.
39. Simioni, P, Tormene, D, Tognin, G, Gavasso, S, Bulato, C, Iacobelli, N P, et al. (2009). X-linked thrombophilia with a mutant factor IX (factor IX Padua). The New England journal of medicine 361: 1671-1675.
40. Mackay, F, and Browning, J L (2002). BAFF: a fundamental survival factor for B cells. Nature reviews Immunology 2: 465-475.
41. Nathwani, AC, Reiss, UM, Tuddenham, EG, Rosales, C, Chowdary, P, McIntosh, J, et al. (2014). Long-term safety and efficacy of factor IX gene therapy in hemophilia B. The New England journal of medicine 371: 1994-2004.
42. Nathwani, AC, Tuddenham, EG, Rangarajan, S, Rosales, C, McIntosh, J, Linch, D C, et al. (2011). Adenovirus-associated virus vector-mediated gene transfer in hemophilia B. The New England journal of medicine 365: 2357-2365.
43. Calcedo, R, Morizono, H, Wang, L, McCarter, R, He, J, Jones, D, et al. (2011). Adeno-associated virus antibody profiles in newborns, children, and adolescents. Clinical and vaccine immunology: CVI 18: 1586-1588.
44. Li, C, Narkbunnam, N, Samulski, RJ, Asokan, A, Hu, G, Jacobson, L J, et al. (2012). Neutralizing antibodies against adeno-associated virus examined prospectively in pediatric patients with hemophilia. Gene therapy 19: 288-294.
45. Boutin, S, Monteilhet, V, Veron, P, Leborgne, C, Benveniste, O, Montus, M F, et al. (2010). Prevalence of serum IgG and neutralizing factors against adeno-associated virus (AAV) types 1, 2, 5, 6, 8, and 9 in the healthy population: implications for gene therapy using AAV vectors. Human gene therapy 21: 704-712.
46. Skupsky, J, Zhang, AH, Su, Y, and Scott, DW (2010). B-cell-delivered gene therapy induces functional T regulatory cells and leads to a loss of antigen-specific effector cells. Molecular therapy: the journal of the American Society of Gene Therapy 18: 1527-1535.
47. Lei, T C, and Scott, DW (2005). Induction of tolerance to factor VIII inhibitors by gene therapy with immunodominant A2 and C2 domains presented by B cells as Ig fusion proteins. Blood 105: 4865-4870.
48. Melo, ME, Qian, J, El-Amine, M, Agarwal, RK, Soukhareva, N, Kang, Y, et al. (2002). Gene transfer of Ig-fusion proteins into B cells prevents and treats autoimmune diseases. Journal of immunology 168: 4788-4795.
49. Yu, H, Borsotti, C, Schickel, IN, Zhu, S, Strowig, T, Eynon, E E, et al. (2017). A novel humanized mouse model with significant improvement of class-switched, antigen-specific antibody production. Blood 129: 959-969.
50. Villaudy, J, Schotte, R, Legrand, N, and Spits, H (2014). Critical assessment of human antibody generation in humanized mouse models. Journal of immunological methods 410: 18-27.
51. Lang, J, Kelly, M, Freed, BM, McCarter, MD, Kedl, R M, Torres, R M, et al. (2013). Studies of lymphocyte reconstitution in a humanized mouse model reveal a requirement of T cells for human B cell maturation. Journal of immunology 190: 2090-2101.
52. Cheng, Q, Mumtaz, IM, Khodadadi, L, Radbruch, A, Hoyer, B F, and Hiepe, F (2013). Autoantibodies from long-lived ‘memory’ plasma cells of NZB/W mice drive immune complex nephritis. Annals of the rheumatic diseases 72: 2011-2017.
53. Aurnhammer, C, Haase, M, Muether, N, Hausl, M, Rauschhuber, C, Huber, I, et al. (2012). Universal real-time PCR for the detection and quantification of adeno-associated virus serotype 2-derived inverted terminal repeat sequences. Human gene therapy methods 23: 18-28.

1.-20. (canceled)

21. A method of making plasma cells or plasma cell precursors that express a molecule, the method comprising:
(a) isolating B cells;
(b) developing the B cells;
(c) performing a first round of genome editing of the B cells for protein expression in absence of viral integration;
(d) expanding the B cells; and
(e) differentiating the B cells, optionally, after step (c) or (d), thereby producing plasma cells or plasma cell precursors that express the molecule.

22. The method of claim 21, wherein the molecule is selected from Factor VIII, Factor IX, SERPING1, SERPINA1, complement inhibitory protein, Factor H, Factor I, a C1 inhibitor, an anti-fibrotic molecule, SCGB1A1, a therapeutic antibody or a binding portion thereof, an anti-IL-1 monoclonal antibody, an anti-TNF antibody, an anti-IL-33 antibody, an anti-C5 antibody, an anti-thrombotic molecule, APLN, an anti-PCSK9 inhibitory antibody or binding portion thereof, a neutralizing HIV-1 antibody (bNAbs) or binding portion thereof, IFN-alpha, BAFF, APRIL, IL-10, IL-6, ADAMTS13, LIPA, GLA or ALPL.

23. The method of claim 21, wherein the developing of the B cells is performed after the B cells are arrested at a specific phase of development or activating the B cells such that the B cells are permissive for recombination without further B cell differentiation, wherein the B cell is arrested as an early pro-B cell, a late pro-B cell, a large pre-B cell, a small pre-B cell, an immature B cell, a T1 B cell, a T2 B cell, a marginal-zone B cell, a mature B cell or a memory B cell.

24. The method of claim 21, wherein (c) comprises introducing a single stranded nucleic acid into the B cells.

25. The method of claim 21, wherein (c) is performed by an RNA and protein based transfection, and comprises delivering a nuclease, wherein the nuclease targets at least one genetic loci in the B cell selected from JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1.

26. The method of claim 21, wherein (c) comprises transducing the B cells with a recombinant adeno-associated virus vector comprising a donor template for homologous recombination into a candidate genetic locus.

27. The method of claim 21, further comprising preventing somatic hypermutation of an antibody locus in the B cell.

28. The method of claim 21, further comprising selectively increasing a proportion of gene edited B cells comprising:
(i) performing a second round of genome editing on the B cells to excise a region;
(ii) performing a third round of genome editing on the B cells, wherein the third round of genome editing results in expression of drug activatable growth enhancers;
(iii) RNA transfecting into the B cells short lived drug activatable growth enhancers; and
(iv) inserting genetic modifications that artificially induce non-transformative expansion of gene edited B cells.

29. The method of claim 28, wherein step (i) further comprises removing IgM positive cells.

30. The method of claim 21, further comprising introducing a nucleic acid encoding at least one cell surface protein into the B cells.

31. The method of claim 21, wherein the differentiating step is performed in a three-step culture system comprising an activation and proliferation step, a plasmablast differentiation step, and a plasma cell differentiation step.

32. The method of claim 31, wherein the activation and proliferation step is performed in the presence of any combination of MCD40L (CD40 trimer), CpG oligodeoxynucleotide, IL-2, IL-10 or IL-15, wherein the plasmablast differentiation step is performed in the presence any combination of IL-2, IL-6, IL-10 or IL-15, or wherein the plasma cell differentiation step is performed in the presence any combination of IL-6, IL-15, APRIL or IFNα.

33. The method of claim 21, wherein the plasma cells are long lived plasma cells.

34. The method of claim 21, further comprising purifying the plasma cells by positive selection against CD138.

35. The method of claim 21, further comprising preventing somatic hypermutation by inactivation of an activation-induced cytidine deaminase gene.

36. A composition comprising a plasma cell, wherein the plasma cell is genetically modified to express a molecule selected from an enzyme, neutralizing antibody or binding portion thereof, cytokine, cytokine receptor, complement protein, inhibitory protein, anti-fibrotic molecule, therapeutic antibody or binding portion thereof, anti-thrombotic molecule, glucose response element, or a monoclonal antibody or binding portion thereof.

37. The composition of claim 36, further comprising a second B cell, wherein the second B cell secretes a molecule that induces tolerance of a peptide or that induces engraftment of the plasma cell.

38. The composition of claim 36, wherein the plasma cell comprises an inactivated activation-induced cytidine deaminase gene.

39. A method of expressing a molecule in a subject comprising:
administering the composition of claim 36 to the subject.

40. The method of claim 39, wherein the subject has received a stem cell administration or a solid organ transplantation or is a subject identified or selected as one to receive a stem cell administration or a solid organ transplantation or, wherein the subject has an enzyme deficiency, pulmonary fibrosis, an autoimmune disorder, immune dysregulation, cancer, diabetes, HIV or hypercholesterolemia.
